(19) United States
(12) Patent Application Publication
(10) Pub. No.: US 2024/0117032 A1
(43) Pub. Date: Apr. 11, 2024

(54) NOVEL AFFINITY CONSTRUCTS AND METHODS FOR THEIR USE

(71) Applicants: Heba Mohamed Yassen ABDELGAFFAR, Knoxville, TN (US); Constanze HAHNFELD, Einbeck (DE); Juan Luis JURAT-FUENTES, Knoxville, TN (US); Erik JONGEDIJK, Einbeck (DE); Jeffrey M. STAUB, Wildwood, MD (US); Stefan MELDAU, Einbeck (DE); IMPETUS AGRICULTURE, INC., St. Louis, MO (US)

(72) Inventors: Heba Mohamed Yassen ABDELGAFFAR, Knoxville, TN (US); Constanze HAHNFELD, Einbeck (DE); Juan Luis JURAT-FUENTES, Knoxville, TN (US); Erik JONGEDIJK, Einbeck (DE); JEFFREY M. STAUB, Wildwood, MO (US); Stefan MELDAU, Einbeck (DE)

(21) Appl. No.: 18/259,635

(22) PCT Filed: Feb. 25, 2022
(86) PCT No.: PCT/US2022/017993

§ 371 Date: Jun. 28, 2023

Related U.S. Application Data
(60) Provisional application No. 63/133,386, filed on Jan. 3, 2021.
(60) Provisional application No. 63/241,896, filed on Sep. 8, 2021.

(51) Int. Cl. C07K 16/28; C07K 16/18

(52) U.S. Cl. CPC C07K 16/28; C07K 16/18; C07K 2317/22; C07K 2317/31; C07K 2317/569

(57) ABSTRACT

Affinity constructs to direct insecticidal toxins to insect specific structures of target insects are presented herein. The affinity constructs comprise of at least one affinity molecule that is capable of recognizing, or capable of binding to, or binding to, or being directed to, or being designed to bind to an insect-specific structure in and/or on a target insect, and at least one other affinity molecule capable of binding to, or binding to, or being directed to, or being designed to bind to an insecticidal protein (toxin) wherein the at least two affinity molecules are operably coupled. Presented herein are also methods of making and using these affinity constructs.

### CROSS-REFERENCE TO RELATED APPLICATIONS

This application claims priority from Provisional Application No. 63/133,386 filed Jan. 3, 2021 and 63/241,896 filed Sep. 8, 2021 the entire contents of which are hereby incorporated by reference.

### INCORPORATION BY REFERENCE OF SEQUENCE LISTING

This application contains a Sequence Listing that has been submitted in ASCII format via EFS-Web and is hereby incorporated by reference in its entirety. The ASCII copy, created on Feb. 4, 2022 and is named 111179_717289_Sequence_Listing_ST25.txt and is about 402.9 kilobytes in size.

### FIELD OF THE INVENTION

This invention relates to the field of insecticidal proteins and their targets, the nucleic acid molecules that encode them, as well as compositions and methods for controlling plant pests.

### BACKGROUND OF THE INVENTION

Certain species of microorganisms of the genus Bacillus are known to possess pesticidal activity against a range of insect pests. Bacillus thuringiensis (Bt) is a gram-positive spore forming soil bacterium characterized by its ability to produce crystalline inclusions that are specifically toxic to certain orders and species of plant pests, including insects, but are harmless to plants and other non-target organisms. For this reason, compositions with Bacillus thuringiensis strains, or their insecticidal proteins can be used as environmentally acceptable insecticides to control agricultural insect pests or insect vectors of a variety of human or animal diseases. Crystal (Cry) proteins from Bacillus thuringiensis have potent insecticidal activity against predominantly Lepidoptera, Diptera, Coleoptera, Hemiptera and Nematode pests. Based on this property crop plants have been genetically engineered to produce insecticidal proteins from Bacillus thuringiensis to thereby exhibit enhanced insect resistance. Sprays and surface applications of microbial insecticides provide an environmentally friendly alternative to synthetic chemical pesticides and can be produced in a cost-effective manner.

A serious threat to the continued efficacy of current insecticidal proteins, such as Cry proteins, whether expressed in transgenic plants or applied over the top on crops or on insect pests, is the evolution of resistance in target pests (Tabashnik et al. 2013, Nat Biotechnol 31, 510-521). At least five different insect species have developed resistance to several Bt toxins, such as Cry toxins, in transgenic crops. The most common resistance mechanism is the reduction in toxin binding to midgut cells, that in different insect species include mutations in Cry toxin receptors such as cadherin, aminopeptidase (APN) and alkaline phosphatase (ALP) (reviewed in Pardo-Lopez et al. 2013, FEMS Microbiol. Rev. 37, 3-22).

Current approaches to address resistance require: (i) identification of new toxins or (ii) targeted modification of existing toxins.

About 952 toxin genes, encoding different entomopathogenic proteinaceous toxins, have been identified and characterized in the Bt strains isolated from different regions of the world (www.lifesci.sussex.ac.uk/Home/Neil_Crickmore/Bt/). The toxins have very different activity spectra against various insect classes and nematodes. Therefore, identifying new toxins against specific targets is tedious, often non-targeted and requires large-scale screens with limited probability of success.

Modifications of existing Cry toxins are mainly limited to specific domains that are required for binding to the target sequence, because other modifications may reduce the stability of the toxins, reduce their specificity or interfere with the mechanisms of toxicity, such as pore formation.

However, neither the development of transgenic plants expressing one or more modified insecticidal toxins, nor developing engineered bacterial strains seems to be flexible enough to allow short-term adjustments to the development of resistances in the insect pest or changes in the pest spectrum. The development of transgenic plants is often very time-consuming and may take up to at least 10 years, thus ruling out modifications or adaptations on a short time scale. Further, and this is true also for bacterial strains that are applied to the surface of plants, alternatives are often missing once a given pest has developed tolerance or even resistance to a certain bacterial toxin.

There remains a need to develop new and effective pest control agents that provide an economic benefit to farmers and that are environmentally acceptable. Particularly needed are control agents that can target to a wider spectrum of insect pests and that efficiently control insect strains that are or could become resistant to existing insect control agents.

### SUMMARY OF THE INVENTION

One aspect of the present disclosure encompasses an affinity construct comprising (1) at least one affinity molecule A capable of recognizing, or capable of binding to, or binding to, or being directed to, or being designed to bind to an insect-specific structure in and/or on a target insect, and (2) at least one affinity molecule B capable of binding to, or binding to, or being directed to, or being designed to bind to an insecticidal protein (toxin), wherein the at least one affinity molecule A and the at least one affinity molecule B are optionally separated by a linker L comprising at least one amino acid.

In one aspect, the at least one affinity molecule A of the affinity construct is different from the at least one affinity molecule B.

In some aspects the at least one affinity molecule A of the affinity construct has one or more binding sites (valences) for the same or different insect-specific structures in and/or on a target insect and wherein the at least one affinity molecule B has one or more binding sites (valences) for the same or different insecticidal protein (toxins).

In some aspect the at least one affinity molecule A specifically binds to a receptor, more specifically a membrane-bound receptor, of an inner organ of the target insect.

In some aspects the at least one affinity molecule A specifically binds to a membrane-bound receptor of a digestive tract, of a reproductive organ, or of a nervous system.

In some aspect the affinity construct with at least one affinity molecule A specifically binds to a membrane-bound receptor of a digestive tract, of a reproductive organ, or of a nervous system.

In one aspect the at least one affinity molecule A specifically binds to a fragment from an extracellular loop of NAAT protein.

In one aspect the at least one affinity molecule A specifically binds to a portion of FAW cadherin.

In one aspect the at least the at least one affinity molecule A specifically binds to a portion of integral membrane subunit (Vo) protein complex of the V-ATPase.

In one aspect the at least one affinity molecule A specifically binds to an extracellular loop in ABCC1.

In one aspect the at least one affinity molecule A specifically binds to an extracellular portion of venom dipeptidyl peptidase-4-like isoform X1 or peptide transporter family 1 isoform X1.

In some aspects the insecticidal protein (toxin) is selected from the group consisting of crystal toxins (Cry and Cyt proteins), vegetative insecticidal toxins (Vip proteins), mosquitocidal toxins (Mtx proteins), binary toxins (Bin proteins),

Tpp, Mpp, Gpp, App, Spp, Vpa, Vpb, Mcf, Pra, Prb, Xpp, Mpf toxins and secreted insecticidal toxins (Sip proteins), as well as fragments or multimers thereof. In some aspects the insecticidal protein (toxin) is selected from a group consisting of crystal protein toxins derived from Bacillus thuringiensis.

In some aspects the at least one affinity molecule A and the at least one affinity molecule B are coupled by a linker sequence L.

In some aspects the at least one affinity molecule A and the at least one affinity molecule B are an affinity mediating molecule selected from the group consisting of a protein, carbohydrate, lipid or nucleotide, or a fragment, derivative or variant of any of these, wherein the at least one affinity molecule A and the at least one affinity molecule B are identical or different.

In some aspects the affinity molecules A and B are not antibodies or a fragment, derivative or variant thereof.

In some aspects the binding protein is selected from the group consisting of affimers (adhirons), affibodies, affilins, affitins, nanofitin, alphabodies (triple helix coiled coil), anticalins, lipocalins, avimers, DARPins (ankyrin repeat), fynomer, kunitz domain pepties, monobodies, adnectins, trinectins, nanoCLAMPs, cellulose/carbohydrate binding molecule (CBM) (for example, dockerins or lectins), centyrins, pronectins, and fibronectin or a fragment, derivative or variant of any of these.

In some aspects the affinity molecule A comprises a naturally occurring or engineered antibody A or a fragment, derivative or variants thereof and the affinity molecule B comprises a naturally occurring or engineered antibody B or a fragment, derivative or variants thereof that are operably connected by the linker L.

In some aspects the antibody A and antibody B of the affinity construct, each is selected from the group consisting of a Fab fragment, a single heavy chain and a single light chain, a single chain variable fragment, a VHH fragment, CDR3 region and a bispecific monoclonal antibody (diabody).

In some aspects the antibody A and the antibody B, each comprises a single domain antibody or a fragment, derivative or variants thereof, operably connected by the linker L.

In some aspects the antibody A comprises one or more binding sites (valences) for the same membrane-bound receptor of a digestive tract, of a reproductive organ, or of a nervous system of an insect.

In some aspects the antibody A comprises an amino acid sequence selected from any one of SEQ. ID. NOS. 74, 76, 78, 80, 82, 84, 86, 88, 90 and 92.

In some aspects the antibody A comprises a domain that specifically binds to a fragment from an extracellular loop of NAAT protein.

In some aspects the antibody A comprises a domain that specifically binds to a portion of an insect cadherin.

In some aspects the antibody A comprises a domain that specifically binds to a portion of integral membrane subunit (Vo) protein complex of the V-ATPase.

In some aspects the antibody A comprises a domain that specifically binds to an extracellular loop in ABCC1 or ABCC2.

In some aspects the antibody A comprises a domain that specifically binds to an extracellular portion of venom dipeptidyl peptidase-4-like isoform X1 or peptide transporter family 1 isoform X1.

In some aspects the antibody B comprises one or more binding sites (valences) for the same or different insecticidal protein(s) (toxins).

In some aspects the insecticidal protein (toxin) is selected from the group consisting of crystal toxins (Cry and Cyt proteins), vegetative insecticidal toxins (Vip proteins), mosquitocidal toxins (Mtx proteins), Tpp, Mpp, Gpp, App, Spp, Vpa, Vpb, Mcf, Pra, Prb, Xpp, Mpf toxins, binary toxins (Bin proteins), and secreted insecticidal toxins (Sip proteins), as well as fragments or multimers thereof.

In some aspects the insecticidal protein (toxin) comprises a crystal protein toxin derived from Bacillus thuringiensis.

In some aspects the antibody B comprises an amino acid sequence selected from any one of SEQ. ID. NOS. 68, 70 and 72.

In some aspects the linker L comprises an amino acid sequence selected from any one of SEQ. ID. NOS. 54, 56, 58, 60, 62 and 64.

In some aspects the affinity construct comprises an amino acid sequence of any one of 96, 98, 100, 102, 104, 106, 108,110, 112, 114 and 116.

In some aspects the affinity construct comprises an amino acid sequence with at least about 70% sequence identity with any one of SEQ. ID. NOS. 96, 98, 100, 102, 104, 106, 108,110, 112, 114 and 116.

In some aspects the present disclosure encompasses a first recombinant nucleic acid construct, comprising a polynucleotide sequence encoding an amino acid sequence of any one of the affinity constructs of claims 17-33.

In some aspects the nucleic acid sequence is at least about 70% identical with any one of SEQ. ID. NOS. 95, 97, 99, 101, 103, 105, 107, 109, 111, 113 and 115.

In some aspects the polynucleotide sequence encoding the amino acid sequence is codon optimized for expression in a selected host cell.

In some aspects the selected host cell is a yeast cell, a bacterial cell, or a plant cell.

In some aspects the first recombinant nucleic acid further comprises a promoter operably linked to the polynucleotide sequence.

In some aspects the promoter comprises a constitutive promoter, an inducible promoter, a plant specific promoter, a plant tissue specific promoter, a CaMV promoter or a microbial promoter.

In some aspects the present disclosure encompasses a transgenic host cell comprising the first recombinant DNA construct.

In some aspects the present disclosure encompasses a transgenic host cell further comprising one or more nucleic acid sequences, each encoding an insecticidal protein (toxin) that specifically binds to or can be targeted to bind to at least one domain in the at least one affinity molecule B of the affinity construct.

In some aspects the present disclosure encompasses a transgenic host cell wherein the transgenic host cell is a yeast cell, a bacterial cell or a plant cell.

In some aspects the present disclosure encompasses a insecticidal composition comprising the affinity construct as above and at least one insecticidal protein (toxin), wherein the one or more insecticidal protein (toxin) can specifically bind to or can be targeted to bind to at least one domain in the at least one affinity molecule B of the affinity construct.

In some aspects the insecticidal composition further comprises a carrier.

In some aspects the insecticidal composition the carrier may be any one of a powder, a dust, pellets, granules, spray, emulsion, colloid or a solution.

In some aspects the insecticidal composition further comprises an insect food source.

In some aspects the insecticidal composition is specifically toxic to one or more insect pests including insects from orders Isoptera, Blattodea, Orthoptera, Phthiraptera, Thysanoptera, Hemiptera, Hymenoptera, Siphonaptera, Diptera, Coleoptera and Lepidoptera.

In some aspects the insecticidal composition is specifically toxic to one or more insect pests selected from a group consisting of Fall armyworm (FAW, Spodoptera frugiperda), Corn earworm (Helicoverpa zea, CEW) and Diamond back moth (DBM, Plutella xylostella).

In some aspects the present disclosure encompasses the use of the insecticidal compositions as above for preventing damage to a plant, plant part or plant seed by one or more insect pest(s).

In some aspects the present disclosure encompasses a method of preventing damage to a plant, a plant part or plant seed by one or more insect pests, comprising contacting the insect pests with the insecticidal composition as above.

In some aspects the insect pests include insects selected from the orders Isoptera, Blattodea, Orthoptera, Phthiraptera, Thysanoptera, Hemiptera, Hymenoptera, Siphonaptera, Diptera, Coleoptera and Lepidoptera.

In some aspects the present disclosure encompasses an insecticidal kit, comprising the insecticidal composition as above.

In some aspects the insecticidal kit further comprises the host cell producing the affinity construct.

In some aspects the insecticidal kit further comprises the instructions for making and using the kit to prevent damage to a plant, plant part or plant seed by one or more insect pest(s).

In some aspects the present disclosure encompasses a method of protecting a plant or plant parts or plant seeds against one or more insect pest(s) by co-expressing the affinity construct together with one or more insecticidal protein(s) (toxin(s)) in a plant, plant parts or plant seeds, wherein the one or more insecticidal protein (toxin) can specifically bind to or can be targeted to bind to at least one domain in the at least one affinity molecule B of the affinity construct.

In some aspects the present disclosure encompasses a method of protecting a plant or plant parts or plant seeds against one or more insect pest(s) by: a. expressing the affinity construct described above in a plant, plant parts or plant seeds and b. applying the one or more insecticidal protein(s) (toxin(s)) to the plant, plant parts or plant seeds, wherein the one or more insecticidal protein (toxin) can specifically bind to or can be targeted to bind to at least one domain in the at least one affinity molecule B of the affinity construct.

In some aspects the present disclosure encompasses a method of protecting a plant or plant parts or plant seeds against one or more insect pest(s) by: a. applying the affinity construct described above to the plant, plant parts or plant seeds, wherein said affinity construct is expressed in one or more host cell and is applied to said plant, plant parts or plant seeds either in purified form or by applying the microorganism(s) expressing the affinity construct; and b. expressing the one or more insecticidal protein(s) (toxin(s)) in the plant, plant part or plant seed, wherein the one or more insecticidal protein (toxin) can specifically bind to or can be targeted to bind to at least one domain in the at least one affinity molecule B of the affinity construct.

In some aspects the present disclosure encompasses a method of protecting a plant or plant parts or plant seeds against one or more insect pest(s) by: (co-)expressing the affinity construct as described above and one or more insecticidal protein(s) (toxin(s)) in one or more microorganisms and applying the one or more microorganisms (co-)expressing the affinity construct and the one or more insecticidal protein(s) (toxin(s)) either in purified form or together with the respective culture medium/media to a plant, plant parts or plant seeds, wherein the one or more insecticidal protein (toxin) can specifically bind to or can be targeted to bind to at least one domain in the at least one affinity molecule B of the affinity construct, wherein ingestion of the microorganism or culture medium/media by the insect pest causes morbidity to or mortality of the insect pest(s).

In some aspects, the affinity constructs provided herein enhance the activity of Cry1F, Cry1Ab and Cry1Ac against their respective natural target insects as indicated by mortality assays. In some aspects the affinity construct provided herein expands the activity of Cry1F, Cri1Ac and Cry1Ab to previously non-susceptible insects as determined (or as measured) by a mortality assay.

### BRIEF DESCRIPTION OF THE FIGURES

FIG. 1 shows examples of valences of V_{H}Hs (nanobodies) (Jain et al., 2007).

FIG. 2A shows examples for different recombinant V_{H}Hs (valences and specificities).

FIG. 2B shows examples for different recombinant V_{H}Hs with combination of valences and specificities.

FIG. 3 shows a comparison of the structures of a conventional antibody (CA), a heavy chain antibody of camelid origin (CHA) and of a V_{H}H. For V_{H}H the protein structure is also provided. Part of the FIG. is modified according to http://www.structuralbiology.be/chaperones.

FIG. 4 depicts a schematic overview of a possible procedure to obtain (single domain) antibodies derived from immunization with different insect and bacterial antigens. Examples for insect gut or intestine-derived proteins are CAD=cadherin, ALP=alkaline phosphatase, APN=aminopeptidase N. The procedure shown can be applied to any insect-derived molecule.

FIG. 5 shows an insecticidal protein (Toxin) supplied with bivalent antibody (V_{H}H/CDR3_{Toxin}-V_{H}H/CDR3_{CAD}) fusion proteins bind with high specificity to insect gut membrane receptors, leading to increased toxicity against target insects (CAD=cadherin, ALP=alkaline phosphatase, APN=aminopeptidase N).

FIG. 6 shows exemplary GPI-anchored insect midgut proteins as target for single domain antibody-mediated insecticidal protein targeting. Midgut proteins can be isolated and identified via Mass Spectrometry. Proteomic data are screened for proteins with glycosylphophatidyl-inositol (GPI) linked sequence motifs. These proteins are used to produce antibodies, which are then fused to other antibodies raised against insecticidal proteins (“Toxins”). Bimodal antibodies then lead to the accumulation of-insecticidal fusion proteins in the membranes of midguts of target insects, leading to increased target insect mortality.

FIG. 7 provides an example of an amino acid sequence of a V_{H}H domain from dromedary germline (modified from Harmsen et al. 2000, Mol. Immunol. 37, 579-590 (FR=Framework region, CDR=complementarity-determining region).

FIG. 8 shows increasing affinity of pore-forming toxins via antibodies to membrane proteins increases oligomerization, pore formation and toxicity to exemplify the key concept of the present disclosure.

FIG. 9 depicts Cry1Ac combined with V_{H}H Cry1Ac-V_{H}H Chitin synthase binds to BBMV of Cry1Ac-resistant Trichoplusia ni (Cabbage looper, CL) strains.

FIG. 10 shows an overview of the potential applications provided by the present disclosure. The combination of all three boxes offers great potential of the present disclosure for insect pest management.

FIG. 11 depicts a structure of the Cadherin from Trichoplusia ni Gray arrows indicate the entire extracellular domain that can be used for affinity molecule determination. Black arrows indicate sub-domains with the extracellular domain (EC1-12) and most proximal epidermal domain (MPED). White arrows indicate areas within the cadherin that are bound by Cry toxin (according to Badran et al. 2016, Nature, 533, 58-63, and Chen et al. 2014, Arch. Insect Biochem. Physiol. 86(1), 58-71).

FIG. 12 depicts a structure of the Aminopeptidase N from Trichoplusia ni Gray arrows indicate Domain 1, black arrows the Cry-toxin-binding region.

FIG. 13A shows a graphical representation of the results of ELISA-based binding assays using biotin-labeled solubilized Plutella brush border membrane vesicles and NAAT nanobodies.

FIG. 13B shows a graphical representation of the results of ELISA-based binding assays using biotin-labeled solubilized Plutella brush border membrane vesicles and cadherin nanobodies.

FIG. 14 demonstrates that the worms fed with nanobodies in conjunction with Cry1Ac toxin exhibit greater mortality and morbidity in comparison to worms fed on control diet or on Cry1Ac alone.

FIG. 15 shows a protein sequence alignment of the second extracellular loop of sodium-dependent nutrient amino acid 1-like (NAAT) protein depicting conservation across multiple insect species.

FIG. 16 shows a protein sequence alignment depicting conservation of Cadherins across multiple insect species.

FIG. 17A shows the sequence alignment of domain 2 of Cry1Ac, Cry1Ab and Cry1F.

FIG. 17B shows the sequence alignment of domain 2 of Cry1B, Cry1 Da and Cry1F.

### DETAILED DESCRIPTION

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

The present disclosure is drawn to novel affinity constructs and methods for controlling insect pests. The novel affinity constructs of the disclosure comprise at least one affinity molecule A capable of recognizing, or capable of binding to, or binding to, or being directed to, or being designed to bind to, an insect-specific structure in and/or on a target insect, and at least one affinity molecule B capable of binding, or binding to, or is directed to, or is designed to bind to, an insecticidal protein (toxin), wherein the at least one affinity molecule A and the at least one affinity molecule B are optionally separated by a linker L comprising at least one amino acid.

In said novel affinity constructs of the disclosure the at least one affinity molecule A capable of recognizing, or capable of binding to, or binding to, or being directed to, or being designed to bind to, an insect-specific structure in and/or on a target insect is different from the least one affinity molecule B capable of binding to, or binding to, or is directed to, or is designed to bind to, an insecticidal protein (toxin). Thus, the present disclosure encompasses affinity constructs comprising at least one affinity molecule A capable of recognizing, or capable of binding to, or binding to, or being directed to, or being designed to bind to an insect-specific structure in and/or on a target insect, and at least one affinity molecule B capable of binding to, or binding to, or being directed to, or being designed to bind to an insecticidal protein (toxin), wherein the at least one affinity molecule A and the at least one affinity molecule B are different from each other, and wherein the at least one affinity molecule A and the at least one affinity molecule B are optionally separated by a linker L comprising at least one amino acid. The novel affinity constructs comprising at least one affinity molecule A and at least one affinity molecule B can be expressed in a transgenic plant or a microorganism, or be applied as an insecticidal spray, solution or coating to a plant, plant part, plant seed or insect. In both cases, i.e., expression in a transgenic plant or a transgenic microorganism as well as application as an insecticidal spray, solution or coating, the concomitant use of the insecticidal toxin which the at least one affinity molecule B is capable of binding to, or is binding to, or is being directed to, or is being designed to bind to an insecticidal protein (toxin), is required. This means that the insecticidal toxin is either to be co-expressed in the transgenic plant or microorganism or is to be added to the composition containing the affinity construct for the application as a spray, solution, or coating.

The term “specifically toxic” and “specific toxin”, as used herein relates to the specific binding demonstrated by compositions described herein, wherein specific binding of a composition to a receptor in a target insect is incapacitating or lethal to that insect, at a measurably higher rate than any incapacity or lethality caused by exposure of generally comparable but non-target insects exposed to the composition. Specific toxicity of a composition relative to a target insect can be determined using any of many means known to those of ordinary skill in the art for quantifying proportion of an insect sample killed or incapacitated, such as by comparative insect counts or quantifying and comparing target and non-target insect damage to control and test plants. A composition that is “specifically toxic” to a target insect, detectably kills or incapacitates a target insect by a factor of at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold, or more relative to a non-target insect exposed to the same composition. The novel affinity constructs find application in controlling insect pest populations and for producing compositions with insecticidal activity. The novel affinity constructs provided by the present disclosure facilitate the natural function of insecticidal proteins, allow generating new mode of actions by targeting insecticidal proteins to new receptors within the insect pest, and thereby to diminish or overcome insect resistance.

The novel affinity constructs can be generated, for example and as explained in more detail below, by fusing a first affinity molecule or a fragment thereof raised against insect-specific structures (e.g., gut or intestine proteins of target insects) (“affinity molecule A”) with a second affinity molecule B raised against an insecticidal protein (toxin) and thus capable of binding to it, wherein the affinity molecule A and the at least one affinity molecule B are optionally separated by a linker L comprising at least one amino acid and wherein the at least one affinity molecule A and the at least one affinity molecule B are identical or different. The term “raised against” as used herein refers to the specific polypeptide sequence that was used as an antigen to raise affinity molecules for example (but not restricted to) antibody, nanobody, sdAb, V_{H}H, CDR3 etc or design binding partners against.

These novel affinity constructs can be formulated in a composition provided by the present disclosure, wherein the composition further comprises an insecticidal protein (toxin). This insecticidal protein (toxin) corresponds to the insecticidal protein (toxin) which the at least one affinity molecule B of the novel affinity construct is capable of binding to, or is binding to, or is being directed to, or is being designed to bind to. Such compositions exhibit insecticidal activity and, hence, find application in controlling insect pest populations.

The insect is exposed to the affinity construct provided by the present disclosure in combination with an insecticidal protein (toxin) the at least one affinity molecule B is capable of binding to, or is binding to, or is being directed to, or is being designed to bind to. This exposure is realized either (a) by co-expression of the affinity construct and the insecticidal protein (toxin) in a transgenic plant upon which the insect pest is generally feeding, or (b) by co-expression of the affinity construct and the insecticidal protein (toxin) in a transgenic microorganism followed by the application of the microorganism either in purified form or together with the respective culture medium/media to a plant, plant parts or plant seeds upon which the insect pest is generally feeding, or (c) by expressing the affinity construct in a transgenic plant, plant part or plant seed and applying the one or more insecticidal protein (toxin) in purified form or by applying an microorganism expressing the insecticidal protein (toxin) to the transgenic plant, plant part or plant seed, or (d) by expressing the affinity construct in one or more microorganism while expressing the insecticidal protein (toxin) in a plant, plant part or plant seed, and applying the affinity construct being expressed in the one or more microorganism in either purified form or by applying the one or more an microorganism expressing the affinity construct to the plant, plant parts or plant seed expressing the insecticidal toxin (protein); or (e) by formulating the affinity construct and the insecticidal protein (toxin) as an insecticidal composition that is then applied to the plant, plant part or plant seed upon which the insect pest is generally feeding.

The present disclosure encompasses the use of the novel affinity constructs (or the novel insecticidal compositions comprising the novel affinity constructs) for protecting a plant, plant part or plant seed against an insect pest. The methods for protecting plants involve transforming plants or microorganisms with one or more nucleic acid sequences encoding a novel affinity construct provided by the present disclosure and an insecticidal protein, wherein the insecticidal protein (toxin) corresponds to the insecticidal protein (toxin) to which the at least one affinity molecule B of the novel fusion protein is B is capable of binding to, or is binding to, or is being directed to, or is being designed to bind to.

Also encompassed by the present disclosure are methods for making the novel affinity constructs (and nucleic acids encoding the affinity constructs), methods of using same as well as methods for protecting plants, plant parts and plant seeds by means of the novel affinity constructs. The methods for protecting plants involve transforming plants or microorganisms with a nucleic acid sequence encoding a novel affinity molecule provided by the present disclosure and/or with a nucleic acid sequence encoding the insecticidal protein (toxin) to which the at least one affinity molecule B of the novel fusion protein is B is capable of binding to, or is binding to, or is being directed to, or is being designed to bind to.

The methods for protecting plants also involve transforming a microorganism with one or more nucleic acid sequences encoding a novel affinity construct provided by the present disclosure and an insecticidal protein, wherein the insecticidal protein (toxin) corresponds to the insecticidal protein (toxin) to which the at least one affinity molecule B of the novel affinity construct B is capable of binding to, or is binding to, or is being directed to, or is being designed to bind to, and applying either the transformed microorganisms or the purified novel affinity construct expressed in the microorganisms to the plant, plant part or plant seed for uptake by a feeding insect pest.

Also encompassed are transgenic plants, plant parts, plant tissues or plant seed thereof as well as transgenic microorganisms expressing the novel affinity constructs and/or the insecticidal protein (toxin) to which the at least one affinity molecule B of the novel affinity construct B is capable of binding to, or is binding to, or is being directed to, or is being designed to bind to.

### The Affinity Constructs

The present disclosure in particular provides novel affinity constructs comprising multi-specific affinity molecules directed (1) against known or novel insect-specific structures (e.g., receptors) in/or on the insect pest (the “at least one affinity molecule A”) and (2) against an insecticidal protein (toxin) (the “at least one affinity molecule B”). Affinity molecules A and B are combined to bind the insecticidal protein (toxin) on the one hand and to an insect-specific structure (e.g., receptor) on the other hand, thereby increasing the affinity of the insecticidal protein (toxin) to a receptor. This in turn results in (restoration of) binding of the insecticidal protein to its receptor in and/or on the insect pest or to increased activity of the insecticidal protein (toxin). This system provides, amongst others, the advantage that the insecticidal protein (toxin) is not modified itself, therefore decreasing the risk that the insecticidal protein (toxin) may become dysfunctional.

The novel affinity construct provided by the present disclosure exhibits, amongst others, (1) the benefit of more efficient binding of a known insecticidal protein (toxin) to its natural receptors in an insect pest, (2) the benefit of improved targeting of an insecticidal protein (toxin) to both its natural as well as novel receptors in the insect pest, and (3) the benefit of combining multiple mode of actions/site of actions of insecticidal proteins by targeting multiple existing and/or novel receptors in an insect pest. These benefits help to diminish or overcome resistance of an insect pest against the function of an insecticidal protein (toxin), help to expand the range of target insects for a given insecticidal protein (toxin) and help to increase stability of the affinity construct, without being limited thereto. These effects are discussed in more detail in the following.

### Efficient Binding of an Insecticidal Protein (Toxin) to its Natural Receptors in the Insect Pest

The novel affinity construct provides for efficient binding of insecticidal proteins to the respective natural receptors in the insect pest. This helps to facilitate the function of the insecticidal proteins and will diminish or overcome insect resistance. In particular, the novel affinity construct provided by the present disclosure provides a way of increasing the binding efficiency of insecticidal proteins to insect target structures, in particular target structures of an inner organ of an insect, preferably of the digestive tract, a reproductive organ, or the nervous system, more preferably of such as the gut or intestine. Preferably, such target structures are protein receptors or parts of the brush border membrane.

The digestive system of insects comprises an alimentary canal or gut, which can be divided into three sections: foregut, midgut, and hindgut. The novel affinity construct is particularly useful for binding to receptors in the insect foregut, midgut, and hindgut, but preferred in the insect midgut or insect larva midgut. The novel affinity constructs provided by the present disclosure allow delivering and retaining an insecticidal protein to the (surface of the) insect midgut, in particular delivering the insecticidal protein specifically to the area in the insect's midgut, where the impact of said insecticidal protein affinity-bound to the receptor in and/or on a target insect is maximized. Retaining the insecticidal protein on the (surface of the) insect midgut for example has the effect/advantage that oligomerization and pore formation is improved, thereby improving the toxic effect of the insecticidal protein on the insect and, thus, improving the efficacy of the insecticidal protein in controlling the insect population.

Preferably, the affinity molecule A, exhibits a high-avidity specific binding to receptors present on/in the membrane of epithelial cells of the microvilli of the midgut. The affinity molecules are easily internalized by the insect and are easily attached to the midgut (microvilli) antigens, which is sufficient to retain the insecticidal protein by way of the affinity molecule B comprised in the affinity construct of the present disclosure, thereby increasing the efficacy of an insecticidal protein in controlling the insect population. A correlation between the presence of the affinity construct and insecticidal protein on the one hand and bioactivity against target insects on the other hand can be established for the affinity constructs and the methods of the disclosure.

### Improved Targeting of an Insecticidal Protein (Toxin) to Receptors in the Insect Pest

The novel affinity construct provided by the present disclosure further serves to improve targeting of insecticidal proteins to insect pests by increasing the target spectrum of a given insecticidal protein (toxin). This is in particular achieved by, for example, restoration of the binding of an insecticidal protein to its natural receptor(s) in an insect to which this insecticidal protein does not bind anymore (e.g., to restore functionality of a given insecticidal protein whose receptor has changed due to mutation and is not binding the protein anymore), and/or by “arming” an insecticidal protein that formerly was not active in a certain insect species due to the fact that a receptor for that protein is missing. In addition, the affinity molecules can be targeted to known and new receptors (e.g., structures at the brush border membrane of insects and others) from any insect species, thereby increasing the spectrum of insecticidal protein (toxins), like, for example, high activity-Cry proteins and other toxins, to insect species that are otherwise not targeted by these insecticidal proteins (toxins). Targeting these toxins to other receptors/structures in, for example, the insect midgut provides a toxic effect against the insect.

Further, the affinity molecules A comprised in the affinity structure of the present disclosure can be designed in different ways to recognize target structures like, for example, receptor(s), in a target insect pest: (1) two or more affinity molecules A may be designed to recognize the same insect-specific structure in and/or om different target insects, (2) two or more affinity molecules A may be designed to recognize the same insect-specific structure in and/or on different target insects, or (3) two or more affinity molecules A may be designed to recognize different insect-specific structures in the same target insect.

Moreover, by using novel small and stable affinity molecules (e.g., V_{H}Hs, Affimers and the like) that can bind small epitopes of receptor molecules, one can precisely direct the insecticidal protein (toxin) to the target structure. In addition, once a resistance mode of action of an insect species against a certain toxin is known, the present invention further allows quickly designing affinity molecule/toxin combinations that can robustly overcome resistance.

### Facilitation of the Function of an Insecticidal Protein (Toxin) to Diminish or Overcome Insect Resistance

The novel ways of increasing the binding efficiency of insecticidal proteins to their insect-specific target structures described herein specifically serve to counteract resistance of insect pests to insecticidal proteins that is caused by reduced binding of the insecticidal protein (toxin) to cells of the digestive system of an insect pest.

The novel affinity constructs provided by the present disclosure facilitate the function of insecticidal proteins and overcome insect resistance. They can be used for producing compositions with insecticidal activity, and find use in controlling, inhibiting growth of or killing, e.g., Isopteran, Blattodean, Orthopteran, Phthirapteran, Thysanopteran, Hymenopteran, Hemipteran, Siphonapteran, Lepidopteran, Coleopteran, Dipteran, and Hemipteran pest populations. Such insecticidal compositions are encompassed by the present disclosure. The present disclosure encompasses controlling, inhibiting growth of or killing pest populations as aforementioned using the novel affinity construct of the present disclosure and an insecticidal protein (toxin), wherein the insecticidal protein (toxin) corresponds to the insecticidal protein (toxin) to which the at least one affinity molecule B of the novel affinity construct is capable of binding, or is binding to, or against which the at least one affinity molecule B is directed to, or designed to bind to. As mentioned elsewhere herein, the affinity construct of the present disclosure comprises at least two affinity molecules, in particular at least one affinity molecule A, capable of binding, binding to, directed to, or designed to bind to an insect-specific structure in and/or on a target insect, and at least one affinity molecule B capable of binding of binding, binding to, directed to, or designed to bind to an insecticidal protein (toxin). Thus, the at least one affinity molecule A is capable of recognizing, or is capable of binding to, or is binding to, or is being directed to, or is being designed to bind to an insect-specific structure (such as, for example, a receptor) in and/or on a target insect, and at least one affinity molecule B is capable of binding to, or is binding to, or is being directed to, or is being designed to bind an insecticidal protein. In such embodiments where more than one of the affinity molecules A is designed to recognize an insect-specific structure (such as, for example, a receptor) in and/or on a target insect at least three general strategies can be applied: (1) the two or more affinity molecules A capable of recognizing, or capable of binding to, or binding to, or being directed to, or being designed to bind to an insect-specific structure in and/or on a target insect can be capable of recognizing, or capable of binding to, or binding to, or being directed to, or being designed to bind to different insect-specific structures in different target insects (i.e., for example, one affinity molecule designed to bind to insect-specific structure T1 in insect X and one affinity molecule designed to bind to insect-specific structure T2 in insect Y, and so on), (2) the two or more affinity molecules A capable of recognizing, or capable of binding to, or binding to, or being directed to, or being designed to bind to an insect-specific structure in and/or on a target insect can capable of recognizing, or capable of binding to, or binding to, or being directed to, or being designed to bind to the same insect-specific structure in and/or on different target insects (i.e., for example, one affinity molecule designed to bind to insect-specific structure T1 in insect X and one affinity molecule designed to bind to insect-specific structure T1 in insect Y, and so on), or (3) the two or more affinity molecules A capable of recognizing, or capable of binding to, or binding to, or being directed to, or being designed to bind to an insect-specific structure in and/or on a target insect can be capable of recognizing, or capable of binding to, or binding to, or being directed to, or being designed to bind to different insect-specific structures in the same target insect (i.e., for example, one affinity molecule designed to bind to insect-specific structure T1 in insect X and one affinity molecule designed to bind to insect-specific structure T2 in insect X, and so on).

### Increased Stability of the Affinity Constructs

In addition, the novel affinity constructs provided by the present disclosure are considered to have several other distinct properties. In particular, they exhibit a superior relative stability under adverse conditions (e.g., effective under pH extremes, temperature extremes, etc.), which is of importance to make the insect pest control principle work under the varying harsh conditions in insect digestive tracts (e.g., considering the pH variability within the gut/intestine within insect species and pH variability in gut/intestine between insect species). Conventional insecticidal agents such as conventional insecticidal proteins are way inferior in this respect and are often degraded under even less extreme conditions.

The novel affinity constructs provided by the present disclosure allow delivering and retaining an insecticidal protein to the (surface of the) insect gut, in particular delivering the insecticidal protein specifically to the area in the insect's midgut, where the impact of said insecticidal protein affinity-bound to the receptor in and/or on a target insect is maximized. Retaining the insecticidal protein on the (surface of the) insect midgut, for example, has the effect/advantage that oligomerization and pore formation is improved, thereby improving the toxic effect of the insecticidal protein on the insect and, thus, improving the efficacy of the insecticidal protein in controlling the insect population.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art. All technical and scientific terms used herein have the same meaning as commonly understood to one of ordinary skill in the art to which this disclosure belongs unless clearly indicated otherwise.

### Affinity Molecules

Affinity is an attractive interaction between two molecules, that results in a stable association in which the molecules are in close proximity to each other. In this case molecular binding is without building a covalent bond, hence the association is fully reversible. An affinity mediating molecule is a molecule, which is structured in such a way to mediate an interaction with another molecule in a specific although reversible way. Accordingly, the affinity mediating molecules and in particular the affinity molecules according to the present disclosure are molecules showing binding affinity for a target molecule. Specifically, an affinity molecule A according to the present disclosure is a molecule having binding affinity for an insect-specific structure, preferably for a receptor molecule. Receptor molecules which may be targeted by the at least one affinity molecule A of the present disclosure are described elsewhere herein. Further, an affinity molecule B according to the present disclosure is a molecule having binding affinity for an insecticidal protein (toxin). Insecticidal protein (toxin) molecules which may be targeted by the at least one affinity molecule B of the present disclosure are described elsewhere herein.

In the present disclosure, the affinity construct comprises at least two different affinities. At least one of these least two affinities in said affinity construct is affinity molecule A capable of capable of binding to, or binding to, or being directed to, or being designed to bind to an insect-specific structure, preferably for a receptor molecule, in and/or on a target insect, and at least one of these at least two affinities is affinity molecule B capable of binding to, or binding to, or being directed to, or being designed to bind to an insecticidal protein (toxin), wherein the at least one affinity molecule A and the at least one affinity molecule B are optionally separated by a linker L comprising at least one amino acid. Further, the at least one affinity molecule A has been raised or designed against one or more identical or distinct insect-specific structures (e.g., gut or intestine proteins of target insects) and are thus capable of binding to, or are binding to these insect-specific structures, preferably a receptor molecule. Similarly, the at least one second affinity molecule B has been raised or designed against one or more insecticidal proteins (toxins) and are thus capable of binding to, or are binding to such insecticidal proteins.

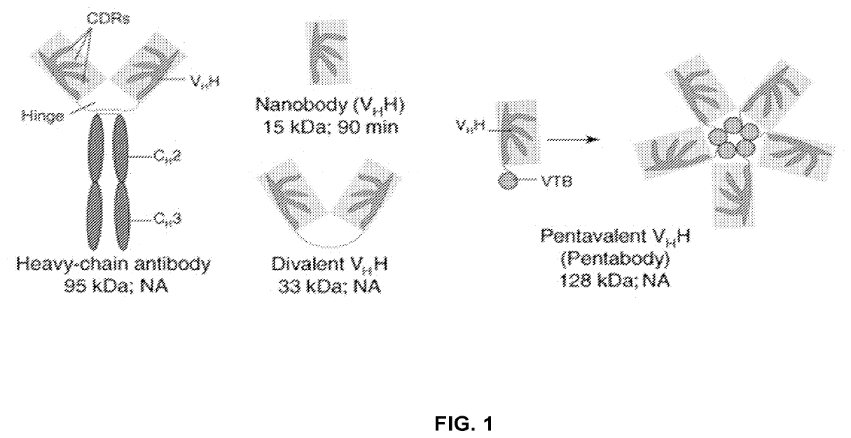

Furthermore, the affinity molecules A and B being comprised in the affinity construct are affinity mediating molecule selected from the group comprising a protein, carbohydrate, lipid or nucleotide, or a fragment, derivative or variant of any of these, wherein the at least one affinity molecule A and the at least one affinity molecule B are identical or different. Proteins encompass a non-antibody binding proteins or antibodies or a fragment, derivative or variant thereof. In some embodiments the non-antibody binding protein is selected from the group comprising affimers (adhirons), affibodies, affilins, affitins, nanofitin, alphabodies (triple helix coiled coil), anticalins, lipocalins, avimers, DARPins (ankyrin repeat), fynomer, kunitz domain pepties, monobodies, adnectins, trinectins, nanoCLAMPs, cellulose/carbohydrate binding molecule (CBM) (for example, dockerins or lectins), centyrins, pronectins, and fibronectin or a fragment, derivative or variant of any of these. The antibody is a naturally occurring antibody or a fragment, derivative or variant thereof, in particular a nanobody or an immunoglobulin gamma (IgG) (see FIGS. 1, 2 and 3). The fragment of the naturally occurring antibody can be an antibody fragment selected from the group comprising a Fab fragment, a single heavy chain and a single light chain, a single chain variable fragment, a V_{H}H fragment, CDR3 region and a bispecific monoclonal antibody (diabody). The Fab fragment can occur as monomer or as a linked dimer, or antibody fragments consisting of a single heavy chain and a single light chain, or consisting of the heavy chain with all three domains, two domains or only on domain of the constant region (the so called crystallizable Fragment Fc) or the single light chain or the so called V_{H}H or the region facilitating the recognition to the antigen comprising the CDR3 region as will be described in more detail further below. Encompassed are also synthetic affinity molecules like three helix coils. The nucleotide is a RNA aptamer, a SOMAmer or a ribozyme or a fragment, derivative or variant thereof.

In the context of the affinity molecule comprising at least one affinity molecule A and at least one affinity molecule B as described above, “capable of binding to, or binding to, or being directed to, or being designed to bind to an insect-specific structure”, preferably for a receptor molecule, in and/or on a target insect encompasses binding of the at least one affinity molecule A to said insect-specific structure, preferably to said receptor molecule, in and/or on a target insect. Thus, in the context of the affinity construct comprising at least one affinity molecule A and at least one affinity molecule B, the at least one affinity molecule A has affinity, more specifically binding affinity, even more specifically specific binding affinity, for an insect-specific structure in and/or on the target insect. Likewise, in the context of the of the affinity construct comprising at least one affinity molecule A and at least one affinity molecule B, the at least one affinity molecule B has affinity, more specifically binding affinity, even more specifically specific binding affinity, for the insecticidal protein (toxin).

As used herein, the terms “specific binding” and “specific binding affinity” when used to characterize any affinity molecule described herein, describes that ability of an affinity molecule to recognize and link to a certain target sequence or structure, i.e., binding partner, such that the linking or binding of the affinity molecule to the target is measurably higher than the binding affinity of the same molecule to a generally comparable, but non-target structure or sequence. The binding affinity of an affinity molecule to target structure or sequence can be determined using any of many means known to those of ordinary skill in the art. A binding domain of an affinity molecule that “specifically binds” to a binding partner, detectably binds the binding partner by a factor of at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold, or more relative to the same molecule binding to a non-target, non-binding partner. The equilibrium dissociation constant (Kd) of any affinity molecule for two or more binding partners can be readily determined and compared to quantify the binding specificity of the affinity molecule of interest with respect to a binding partner, or target of interest. Binding of an affinity molecule to a target structure or sequence can be measured and detected in a variety of ways known in the art, including but not limited to assays using enzymatic or fluorescent labels, radiolabels, gel shift assays, surface plasmon resonance (SPR), biolayer interferometry (BLI), and enzyme linked immunosorbent assays (ELISA). In the context of the affinity construct comprising at least one affinity molecule A and at least one affinity molecule B, the at least one affinity molecule A having affinity, more specifically binding affinity, for an insect-specific structure in and/or on a target insect, is different from the least one affinity molecule B having affinity, more specifically binding affinity, for an insecticidal protein (toxin).

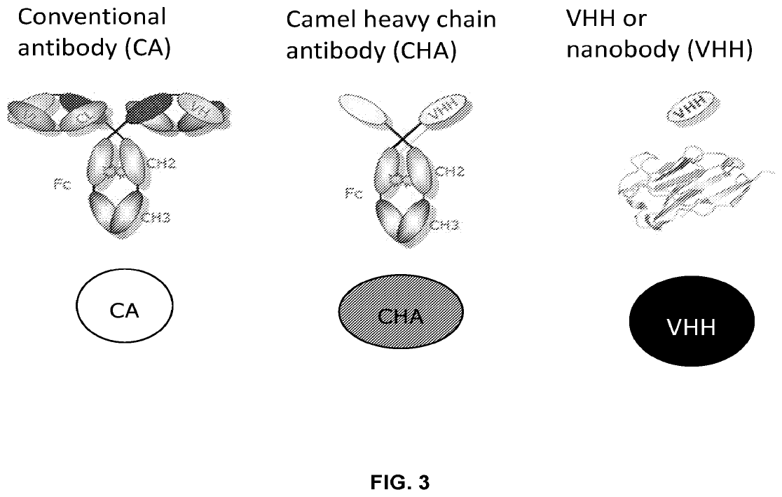

The at least one affinity molecule A of the novel affinity construct may be at least one protein, carbohydrate, lipid or nucleotide, or a fragment, derivative or variant of any of these (or at least two proteins, carbohydrates, lipids or nucleotides, or fragments or derivatives or variants thereof). Preferably, the protein is a non-antibody binding protein or an antibody or a fragment, derivative or variant thereof. More preferred, the non-antibody binding protein is any one of affimers (adhirons), affibodies, affilins, affitins, nanofitin, alphabodies (triple helix coiled coil), anticalins, lipocalins, avimers, DARPins (ankyrin repeat), fynomer, kunitz domain pepties, monobodies, adnectins, trinectins, nanoCLAMPs, cellulose/carbohydrate binding molecule (CBM) (for example, dockerins or lectins), centyrins, pronectins, and fibronectin or a fragment, derivative or variant of any of these. In other embodiments, the antibody is a naturally occurring antibody or a fragment, derivative or variant thereof, in particular a single-domain antibody (sdAb) or a nanobody or an immunoglobulin gamma (IgG) (see FIG. 1 and FIG. 3 for some examples). Preferably, the fragment of the naturally occurring antibody can be an antibody fragment selected from the group comprising a Fab fragment, a single heavy chain and a single light chain, a single chain variable fragment, a V_{H}H fragment, CDR3 region and a bispecific monoclonal antibody (diabody). The Fab fragment can occur as monomer or as a linked dimer, or antibody fragments consisting of a single heavy chain and a single light chain, or consisting of the heavy chain with all three domains, two domains or only on domain of the constant region (the so called crystallizable Fragment Fc) or the single light chain or the V_{H}H or the region facilitating the recognition to the antigen comprising the CDR3 region as will be described in more detail further below. Encompassed are also synthetic affinity molecules like three helix coils. In other preferred embodiments, the nucleotide is a RNA aptamer, a SOMAmer or a ribozyme or a fragment, derivative or variant thereof.

In other preferred embodiments, the at least one affinity molecule A of the novel affinity construct, or a fragment or derivative or variant thereof, is at least one protein, carbohydrate, lipid or nucleotide, or a fragment, derivative or variant of any of these (or at least two proteins, carbohydrates, lipids or nucleotides, or fragments or derivatives or variants thereof).

Likewise, in the present disclosure, the at least one affinity molecule B of the novel affinity construct is a non-antibody binding protein or an antibody or a fragment, derivative or variant thereof. More preferred, the non-antibody binding protein is any one of affimers (adhirons), affibodies, affilins, affitins, nanofitin, alphabodies (triple helix coiled coil), anticalins, lipocalins, avimers, DARPins (ankyrin repeat), fynomer, kunitz domain pepties, monobodies, adnectins, trinectins, nanoCLAMPs, cellulose/carbohydrate binding molecule (CBM) (for example, dockerins or lectins), centyrins, pronectins, and fibronectin or a fragment, derivative or variant of any of these. In other embodiments, the antibody is a naturally occurring antibody or a fragment, derivative or variant thereof, in particular a nanobody or an immunoglobulin gamma (IgG). Preferably, the fragment of the naturally occurring antibody can be an antibody fragment selected from the group comprising a Fab fragment, a single heavy chain and a single light chain, a single chain variable fragment, a V_{H}H fragment, CDR3 region and a bispecific monoclonal antibody (diabody). The Fab fragment can occur as monomer or as a linked dimer, or antibody fragments consisting of a single heavy chain and a single light chain, or consisting of the heavy chain with all three domains, two domains or only one domain of the constant region (the so called crystallizable Fragment Fc) or the single light chain or the V_{H}H or the region facilitating the recognition to the antigen comprising the CDR3 region as will be described in more detail further below. Encompassed are also synthetic affinity molecules like three helix coils. In other preferred embodiments, the nucleotide is a RNA aptamer, a SOMAmer or a ribozyme or a fragment or derivative or variant thereof. More preferably, the antibody is a single domain antibody (sdAb) or a fragment or derivative or variant thereof.

In other preferred embodiments, the at least one affinity molecule B of the novel affinity constructs, or a fragment or derivative or variant thereof, is at least one alphabody or fragment or derivative or variant thereof (or at least two alphabodies or fragments or derivatives or variants thereof).

In some embodiments, the at least one affinity molecule A and the at least one affinity molecule B being comprised in the affinity construct are separated by a linker L comprising at least one amino acid.

In the context of the affinity construct of the present disclosure comprising at least one affinity molecule A and at least one affinity molecule B, the linker may be any amino acid molecule of variable length (minimum length being 1 amino acid) that serves to link the at least one affinity molecule A and the at least one affinity molecule B and that is not causing steric hindrance between the affinity molecules linked by the linker. Preferably, the linker is a molecule that is used to connect the variable domains of the heavy (V_{H}) and light chains (V_{L}) with their respective non-variable domains of immunoglobulins to construct a single chain antibody (scFv), or to engineer bivalent single chain variable fragments (bi-scFvs) by linking two scFvs. Other examples of suitable linkers to be used in the context of the above-mentioned affinity construct comprising at least one affinity molecule A and at least one affinity molecule B are those used in immunotoxins (see, for example, Huston et al. 1992, Biophys J 62, 87-91; Takkinen et al. 1991). Linkers suitable for use in the context of the above-mentioned affinity construct comprising at least one affinity molecule A and at least one affinity molecule B can also be based on hinge regions of antibody molecules (see, for example, Pack and Plückthun 1992; Pack et al. 1993), or be based on peptide sequences found between structural domains of proteins. Fusions can be made between the multivalent affinity molecules at both sides, the C- and the N-terminus. Linkers suitable for use in the context of the novel affinity construct of the present disclosure are also described elsewhere herein, without being limited thereto.

In the context of the present invention, the affinity constructs comprising at least one affinity molecule A and at least one affinity molecule B, can have different valences as described further below.

In the context of the present invention an affinity construct comprising one affinity molecule A and one affinity molecule B, this affinity construct is the simplest form of the affinity construct of the present disclosure. It represents a bispecific fusion protein, since each affinity molecule A and B recognizes one independent target, respectively, i.e. the target of affinity molecule A and the target of affinity molecule B.

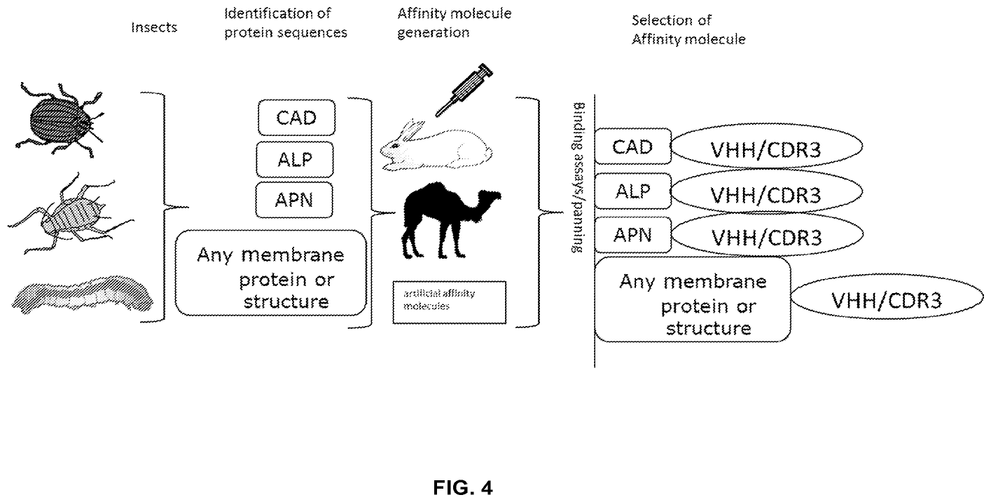

In preferred embodiments, the one or more affinity molecule A and/or the one or more affinity molecule B comprised in the affinity construct of the present disclosure are affinity mediating molecules selected from the group comprising a protein, carbohydrate, lipid or nucleotide, or a fragment, derivative or variant of any of these, wherein the at least one affinity molecule A and the at least one affinity molecule B are identical or different. Preferably, the protein is a non-antibody binding protein or an antibody or a fragment, derivative or variant thereof. More preferred, the non-antibody binding protein is any one of affimers (adhirons), affibodies, affilins, affitins, nanofitin, alphabodies (triple helix coiled coil), anticalins, lipocalins, avimers, DARPins (ankyrin repeat), fynomer, kunitz domain pepties, monobodies, adnectins, trinectins, nanoCLAMPs, cellulose/carbohydrate binding molecule (CBM) (for example, dockerins or lectins), centyrins, pronectins, and fibronectin or a fragment, derivative or variant of any of these. In other embodiments, the antibody is a naturally occurring antibody or a fragment, derivative or variant thereof, in particular a nanobody or an immunoglobulin gamma (IgG). Preferably, the fragment of the naturally occurring antibody can be an antibody fragment selected from the group comprising a Fab fragment, a single heavy chain and a single light chain, a single chain variable fragment, a V_{H}H fragment, CDR3 region and a bispecific monoclonal antibody (diabody). The Fab fragment can occur as monomer or as a linked dimer, or antibody fragments consisting of a single heavy chain and a single light chain or consisting of the heavy chain with all three domains (so called V_{H}H), two domains or only on domain of the constant region (the so called crystallizable Fragment Fc) or the single light chain or the region facilitating the recognition to the antigen comprising the CDR3 region as will be described in more detail further below (see FIG. 4). Encompassed are also synthetic affinity molecules like three helix coils. In other preferred embodiments, the nucleotide is a RNA aptamer, a SOMAmer or a ribozyme or a fragment, derivative or variant thereof.

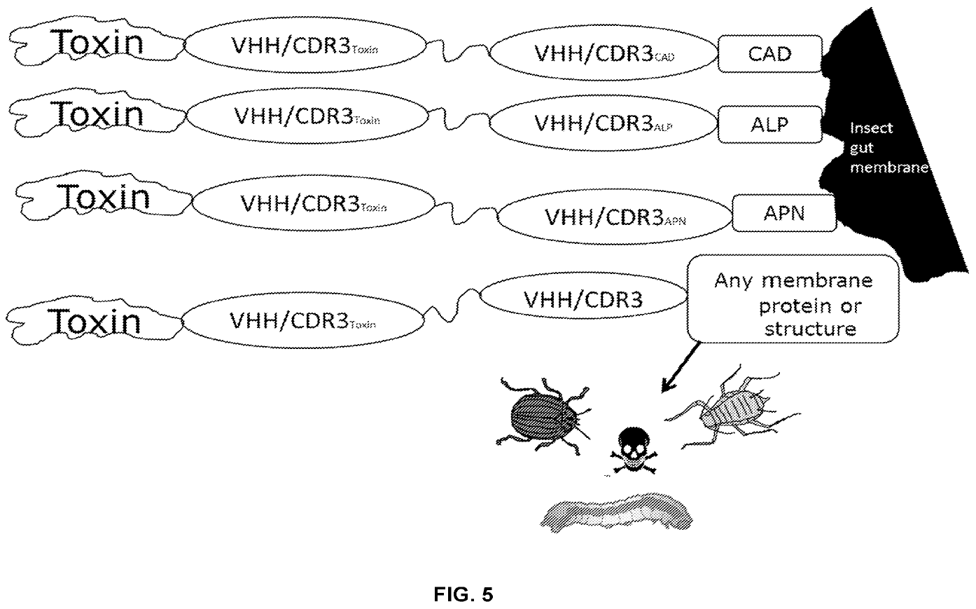

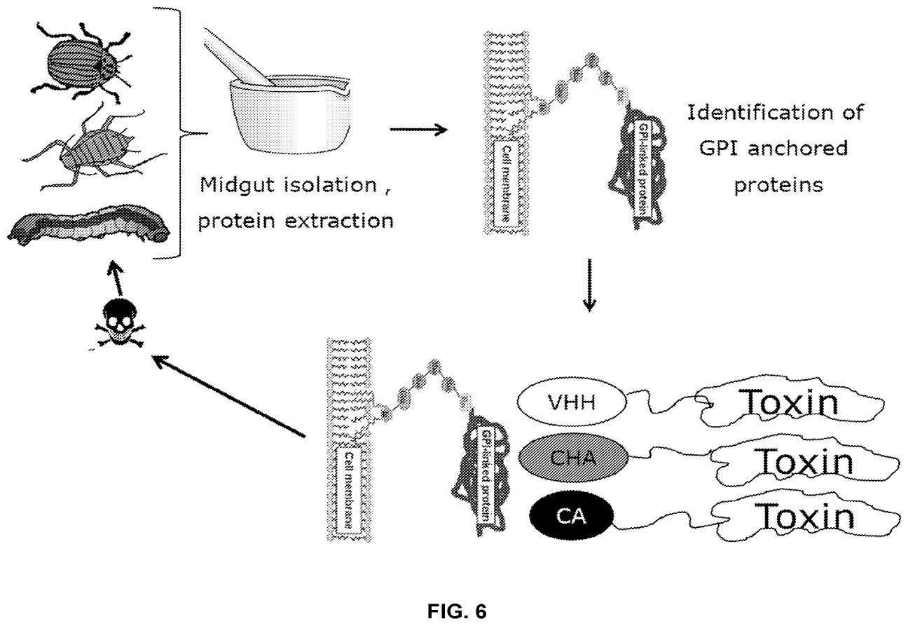

In a further preferred embodiment, the one or more affinity molecule A and/or the one or more affinity molecule B comprised in the affinity construct of the present disclosure are affinity mediating molecules as described above or fragments thereof, wherein at least one of said at least two affinity mediating molecules specifically binds to an inner organ of an insect, preferably to the digestive tract, a reproductive organ or the nervous system, more preferably to the gut or intestine of an insect, and wherein the other of said at least two are affinity mediating molecules binds an insecticidal protein (toxin), wherein the at least two are affinity mediating molecules are optionally separated by a linker L comprising at least one amino acid, and wherein the at least one are affinity mediating molecules specifically binding to the insect-specific structure is different from the least one are affinity mediating molecules binding an insecticidal protein (toxin) (see FIGS. 5 and 6). More preferably, the affinity mediating molecules or fragment thereof specifically binding to an inner organ of an insect, to the digestive tract, a reproductive organ or the nervous system, to the gut or intestine of an insect, specifically binds to a membrane-bound molecule of an inner organ of an insect, preferably of the digestive tract, a reproductive organ or the nervous system, more preferably of the gut or intestine of the insect. Preferably, the membrane-bound molecule is a receptor molecule, preferably an essential receptor molecule, more preferably a receptor molecule for a Cry protein. More preferably, the receptor molecule is selected from the group consisting of cadherin protein receptors, aminopeptidase N protein receptors, alkaline phosphatase protein receptors, ABC transporter protein receptors, chitin synthase B proteins, and 250 kDa protein receptors. In other embodiments the one or more affinity molecule A is targeted against insecticidal structures that in nature do not yet serve as receptors, for example, for insecticidal proteins such as, for example, membrane proteins or proteins that are associated to the membrane or interact with membrane proteins, or to modifications of such proteins (e.g., glycosyl, lipoyl, sumoyl, ubiquitin, phophate residues).

In further preferred embodiments, the affinity construct of the present disclosure comprises one or more affinity molecules A targeted against an insect-specific structure in and/or on a target insect. In that regard, several strategies may by applied if two or more affinity molecules A are incorporated into the affinity construct: (1) the two or more affinity molecules A may be designed to recognize different insect-specific structures in different target insects, (2) the two or more affinity molecules A may be designed to recognize the same insect-specific structure in and/or on different target insects, or (3) the two or more affinity molecules A may be designed to recognize different insect-specific structures in the same target insect.

In some embodiments, where two or more affinity molecules A are present in the affinity construct, the insect-specific structures in and/or on a target insect the at least two affinity molecules A are capable of recognizing, or capable of binding to, or binding to, or being directed to, or being designed to bind to may be identical or distinct. Similarly, in other embodiments, where two or more affinity molecules B are present in the affinity construct, the insecticidal protein (toxins) the at least two affinity molecules B are capable of recognizing, or capable of binding to, or binding to, or being directed to, or being designed to bind to may be identical or distinct.

Similarly, in various embodiments where the affinity construct comprises two or more affinity molecules B targeted against one or more insecticidal proteins (toxins) as mentioned herein, the two or more affinity molecules B are designed according to one of the following three potential strategies (1) the two or more affinity molecules B are capable of recognizing, or capable of binding to, or binding to, or being directed to, or being designed to bind to either the same or different epitopes of the same insecticidal protein (toxin), or (2) the two or more affinity molecules B are capable of recognizing, or capable of binding to, or binding to, or being directed to, or being designed to bind to either the same or different epitopes of different insecticidal proteins (toxins). These strategies include examples where different types of affinity molecule B are employed which are capable of recognizing, or capable of binding to, or binding to, or being directed to, or being designed to bind to the same epitope of the same insecticidal protein (toxin). In such an approach, for example, the first affinity molecule B is a nanobody and the second affinity molecule B is an affimer. In another preferred embodiment where the same epitope of the different insecticidal proteins (toxins) is to be targeted by more than one affinity molecule B, the affinity structure of the present invention is comprising more than one identical affinity molecule B which is capable of recognizing, or capable of binding to, or binding to, or being directed to, or being designed to bind to the same epitope of the different insecticidal proteins (toxins).

In further preferred embodiments, the affinity construct of the present invention is of the structure (AₘLₙBₒ)ₚV_{q} and comprises at least one affinity molecule A, at least one affinity molecule B, optionally a linker L that is separating affinity molecules A and B and optionally a linker V. In these embodiments the integer m is at least 1, the integer n 0 or larger, the integer o at least 1, the integer p at least 1, and the integer q 0 or larger, respectively. The linker V may be any amino acid molecule of variable length (minimum length being 1 amino acid) that serves to link two units AₘLₙBₒ (in embodiment where more than one of these units are present in the affinity construct) consisting of at least one affinity molecule A, at least one affinity molecule B and optionally a linker L and that prevents steric hindrance between these units linked by the linker V. In all these embodiments the affinity molecules A and B and the Linker L, respectively, are as defined above (see Table 1).

TABLE 1
Non-limiting schematic description of different preferred
embodiments of the affinity constructs of the present invention
with reference to the structure (AₘLₙBₒ)ₚV_{q}.

| Affinity | Inte- | Inte- | Inte- | Inte- | Inte- | Corresponding |
|---|---|---|---|---|---|---|
| structure | ger | ger | ger | ger | ger | formula |
| (example) | m | n | o | p | q | (AₘLₙB₀)ₚV_{q} |
| AB | 1 | 0 | 1 | 1 | 0 | (A₁L₀B₁)₁V₀ |
| ALB | 1 | 1 | 1 | 1 | 0 | (A₁L₁B₁)₁V₀ |
| AAB | 2 | 0 | 1 | 1 | 0 | (A₂L₀B₁)₁V₀ |
| AAAB | 3 | 0 | 1 | 1 | 0 | (A3L₀B₁)₁V₀ |
| AALB | 2 | 0 | 1 | 1 | 0 | (A₂L₁B₁)₁V₀ |
| ABB | 1 | 0 | 2 | 1 | 0 | (A₁L₀B₂)₁V₀ |
| ALBB | 1 | 1 | 2 | 1 | 0 | (A₁L₁B₂)₁V₀ |
| AABB | 2 | 0 | 2 | 1 | 0 | (A₂L₀B₂)₁V₀ |
| ABAB | 1 | 0 | 1 | 2 | 0 | (A₁L₀B₁)₂V₀ |
| ABVAB | 1 | 0 | 1 | 2 | 1 | (A₁L₀B₁)₂V₁ |
| ALBVALB | 1 | 1 | 1 | 2 | 1 | (A₁L₁B₁)₂V₁ |
| AABVAAB | 2 | 0 | 1 | 2 | 1 | (A₂L₀B₁)₂V₁ |
| ABABAB | 1 | 0 | 1 | 3 | 0 | (A₁L₀B₁)₃V₀ |

These embodiments cover affinity constructs comprising any combination of one or more units AₘLₙBₒ (with the linker L being present (i.e., having an amino acid length of at least 0) or not) which are optionally linked by the linker V.

### Preferred Affinity Molecules

The affinity molecules comprised in the affinity construct of the present disclosure as well as fragments or derivatives or variants thereof can be both naturally occurring or naturally produced affinity molecules as well as those produced synthetically, for example through in silico/in vitro combinatorial approaches, in silico/in vitro evolutionary approaches and/or other “synthetic” approaches.

Particularly preferred affinity molecules comprised in the affinity construct of to the present disclosure (including the at least one affinity molecule A and the at least one affinity molecule B of the novel affinity construct) is an affinity mediating molecule selected from the group comprising a protein, carbohydrate, lipid or nucleotide, or a fragment, derivative or variant of any of these. As described already above, an affinity mediating molecule is a molecule, which is structured in such a way to mediate an interaction with another molecule in a specific although reversible way. Accordingly, the affinity mediating molecules and in particular the affinity molecules according to the present disclosure are molecules showing binding affinity for a target molecule.

In various embodiments, proteins encompass a non-antibody binding proteins or antibodies or a fragment, derivative or variant thereof. In other embodiments the non-antibody binding protein is selected from the group comprising affimers (adhirons), affibodies, affilins, affitins, nanofitin, alphabodies (triple helix coiled coil), anticalins, lipocalins, avimers, DARPins (ankyrin repeat), fynomer, kunitz domain pepties, monobodies, adnectins, trinectins, nanoCLAMPs, cellulose/carbohydrate binding molecule (CBM) (for example, dockerins or lectins), centyrins, pronectins, and fibronectin or a fragment, derivative or variant of any of these.

The present disclosure also encompasses affinity constructs comprising artificial binding moieties/proteins/molecules and antibody mimetics, selected for example from the group consisting of so called affibody molecules, affilins, affimers, affitins, alphabodies, anticalins, avimers, DARPins, Fynomers, Kunitz domain peptides and monobodies that are derived from single domain antibodies or fragments thereof and have the ability to bind specifically to an antigen. Such artificial binding moieties/proteins/molecules and antibody mimetics exhibit the same binding properties/specificities as the single domain antibodies or fragments thereof of the present disclosure. In various embodiments, artificial binding moieties/proteins/molecules and antibody mimetics of the present disclosure are derived from single domain antibodies or fragments thereof that are of shark or camelid origin.

An antibody mimetic may be considered as an organic compound that, like a single domain antibody of the present disclosure, can specifically bind antigens, in particular receptor molecules as described herein. Antibody mimetics according to the present disclosure may be considered as molecules that are synthetically composed of nucleic acids or proteins to produce an artificial antibody. An antibody mimetic can be an artificial peptide with a molar mass of about 3 to 20 kDa. In various embodiments, the antibody mimetic comprises an intrabody, a monobody, a linear peptide, or an alphabody. In preferred embodiments, the antibody mimetic is an alphabody. Alphabodies are small proteins (about 10 kDa molecular weight) engineered to bind to a variety of antigens, and the standard alphabody scaffold contains three alpha-helices connected via glycine/serine-rich linkers. Alphabody sequences were found to fold as antiparallel triple-stranded α-helical coiled-coil structures, thus adopting a previously unknown fold (Desmet et al. 2014, Nature Communications 5:5237, DOI:10.1038/ncomms6237).

In various embodiments of the present disclosure, the affinity construct of the disclosure comprises more than one affinity mediating molecule. For example, the present disclosure encompasses a mixture of single domain antibodies (preferably V_{H}Hs of heavy chain-only antibodies) and/or CDR3 loops (molecular stacks).

The antibody is a naturally occurring antibody or a fragment, derivative or variant thereof, in particular a nanobody or an immunoglobulin gamma (IgG).

As used herein, a single domain antibody (sdAb) is an antibody fragment consisting of a monomeric variable domain of an antibody. Thus, in the present disclosure the terms “single domain antibody” and “monomeric variable domain antibody” or “single variable domain antibody” may be used interchangeably. Also, the terms “monomeric variable domain” and “single variable domain” may be used herein interchangeably.

In various embodiments, a single domain antibody is a monomeric variable domain (or a single variable domain) of a heavy chain-only antibody. Heavy chain-only antibodies (hcAbs), also simply called heavy chain antibodies, are found in camelids and cartilaginous fish such as sharks. Heavy chain-only antibodies contain a single variable domain (V_{H}H) and two constant domains (C_{H}2, C_{H}3), i.e., they lack the C_{H}1 constant domain, which is found in a conventional antibody and associates with the light chain and to a lesser degree interacts with the V_{H} domain. The heavy chain antibodies found in cartilaginous fish were originally designated as “immunoglobulin new antigen receptors” (IgNAR), and the single domain antibody obtained from an IgNAR was originally called “variable new antigen receptor (VNAR) fragment”. Like a whole antibody, a single domain antibody is able to bind selectively to a specific antigen. A single domain antibody as used in the present disclosure is a monomeric variable domain of a heavy chain-only antibody as found in camelids or cartilaginous fish, specifically sharks. As used herein, single domain antibodies from heavy chain-only antibodies may also be called V_{H}H fragments or V_{H}H domain antibodies or V_{H}H domains.

In various aspects of the present disclosure, a single domain antibody is any one of a Nanobody™ (also known as nanoantibody; see, for example, www.ablynx.com), an antigen-binding domain of a heavy chain-only antibody, and a V_{H}H, or fragments thereof.

In other aspects of the present disclosure, a single domain antibody encompasses a monomeric variable domain from a conventional Immunoglobulin (Ig), i.e., a monomeric variable domain that is obtained when the dimeric variable domains from a common Ig (e.g., from a mammalian organism such as, e.g., human or mice) have been split into monomers and those are isolated. Thus, in the present disclosure, a single domain antibody encompasses not only a monomeric heavy chain variable domain, but also a monomeric light chain variable domain, or fragments or derivatives or variants thereof. A single domain antibody derived from light chains also specifically binds to target antigens or target epitopes. Thus, in the present disclosure the “single domain antibody” preferably is a “single heavy chain variable domain antibody” or a “single light chain variable domain antibody”. In the present disclosure, the terms “single heavy/light chain variable domain antibody” and “monomeric heavy/light chain variable domain antibody” may be used interchangeably.

In various aspects of the present disclosure, the “single domain antibody” can also be called a “Nanobody™” or a “nanoantibody”. As used herein, a Nanobody™ (or nanoantibody) is an antibody fragment consisting of a monomeric variable domain of an antibody. Thus, in the present disclosure the terms “single domain antibody” and “Nanobody™” or “nanoantibody” may be used interchangeably. In the present disclosure, “single domain antibody” encompasses not only a monomeric heavy chain variable domain, but also a monomeric light chain variable domain. Preferably, a single domain antibody is a monomeric variable domain of a heavy chain-only antibody as found in camelids and cartilaginous fish such as sharks. Thus, in the present disclosure the single domain antibody or fragment or derivative or variant thereof preferably is of shark or camelid origin.

Single domain antibodies are as specific as regular antibodies. As well, they are isolated using standard procedures such as phage panning, allowing them to be cultured in vitro in high concentrations.

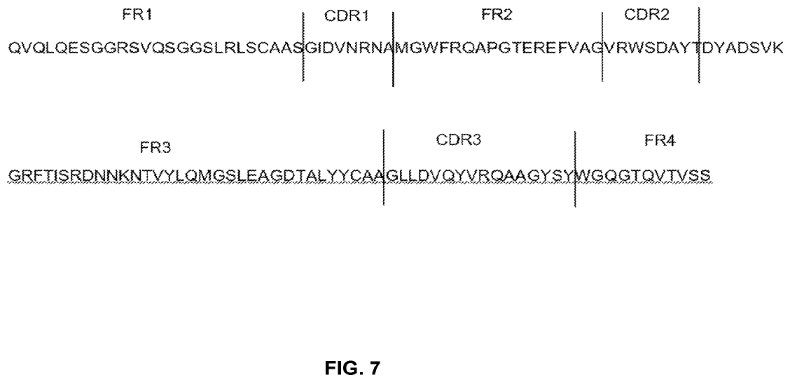

Instead of using entire single domain antibodies (V_{H}H domains), a binding fragment of the sdAb like the extruding CDR3 loops (complementary determining region; region determining binding affinity) of V_{H}H domains can be used as affinity molecule in the affinity construct of the present disclosure. The CDR3 loop of certain single domain antibodies has been found to be much longer than that of conventional variable heavy chain (V_{H}) domains (see FIG. 7, for review, see, e.g., S. Muyldermans 2001, Reviews in Molecular Biotechnology 74, 277-302, or M. M. Harmsen et al. 2000, Molecular Immunology 37, 579-590). Thus, the CDR3 loop region of certain single domain antibody possesses the capacity to form long finger-like extensions that can extend into cavities of antigens, e.g., the active site slot of enzymes. The small size of CDR3 loops reduces the risk of conformational changes and steric hindrance when used in fusions with other proteins. CDR3 loops comprise the epitope-recognizing regions of a single domain antibody which recognize and bind to the antigen. These regions are often sufficient for mediating binding to target proteins. Therefore, the present disclosure encompasses not only the use of complete single domain antibodies as affinity molecule in the affinity construct of the present disclosure, but also the use of functional fragments thereof. Such functional fragments are generally the epitope-recognizing regions of a single domain antibody, e.g., the CDR3 loop of an sdAb of the present disclosure, and they are encompassed for use in an affinity construct of the disclosure. In various embodiments, the functional fragment of a single domain antibody or of a fragment thereof of the present disclosure is the CDR3 loop of a single domain antibody. In various embodiments, the CDR3 loop is derived from of a single domain antibody found in camelids and cartilaginous fish such as sharks.

In various embodiments, the single domain antibodies of the affinity constructs provided by the present disclosure are antibody fragments of a V_{H}H fusion protein, e.g. “one gene encoded single-chain variable domain fragments” and/or “antibody fragments carrying the three CDR loops CDR1, CDR2 and CDR3” and/or “antibody fragments carrying protruding CDR loops (V_{H}H CDR3 like loops) derived from or inspired by V_{H}H fragments obtained from camelid/shark single chain antibodies.

A further advantage of the novel affinity construct of the present disclosure is that the single domain antibodies used as affinity molecule(s) can be selected not to trigger any immune responses in mammals and human. This is different to conventional antibodies or antibody fragments, which are less suitable for transgenic plant approaches as anticipated in the present disclosure. Where necessary or appropriate, the nucleic acid sequence of single domain antibodies or fragments thereof that are of animal origin, and in particular of shark or camelid origin, can be modified/adapted for expression in plants as described herein elsewhere. Also encompassed by the present disclosure are plant sequences, more preferred corn sequences, that are homologues of sequences of single domain antibodies or fragments thereof that are of animal origin, and in particular of shark or camelid origin. Thus, the present disclosure encompasses identifying homologues in the plant genome of the sequences of single domain antibodies or fragments thereof of the present disclosure that are of a normal origin and in particular of shark or camelid origin. Such homologous sequences identified in the genome of a plant of interest, preferably in the corn genome, can be used for expression of single domain antibodies or fragments thereof in plants.

In the present disclosure, the single domain antibody or a fragment or derivative or variant thereof, e.g., the CDR3 loop of an sdAb, can be monovalent or multivalent, which means in case of the latter that two or more single domain antibodies or fragments or derivatives or variants thereof are fused or linked with each other. Suitable linker molecules are described elsewhere in the present disclosure. For example, the single domain antibody or fragment or derivative or variant thereof, e.g., the CDR3 loop of an sdAb, can be divalent, trivalent, tetravalent, or multivalent.

The affinity molecules and fragments thereof of the present disclosure can be applied to a plant, or a part or seed thereof. Also, the affinity molecules and fragments thereof of the present disclosure can be expressed in a plant, or a part or seed thereof. Methods of producing synthetic affinity molecules are well known to the person skilled in the art.

### Target Binding Structures

The affinity construct of the present disclosure comprises at least two affinity molecules. At least one of these at least two affinity molecules is affinity molecule A capable of recognizing, or capable of binding to, or binding to, or being directed to, or being designed to bind to an insect-specific structure in and/or on a target insect. Further, at least one of these at least two affinity molecules is affinity molecule B capable of binding to, or binding to, or being directed to, or being designed to bind to an insecticidal protein (toxin). The at least one affinity molecule A has been raised or designed against one or more insect-specific structures and is thus capable of binding to or are binding to such structure(s) in the insect pest. Further, the at least one second affinity molecule B has been raised or designed against one or more insecticidal proteins (toxins) and is thus capable of binding to or are binding to such insecticidal protein(s).

### The Insect Specific Structures/Receptors:

In various embodiments of the disclosure, the one or more affinity molecule A or a fragment thereof being comprised in the affinity construct of the present disclosure are capable of recognizing, or capable of binding to, or binding to, or being directed to, or being designed to bind specifically to a membrane-bound molecule of an inner organ of an insect, in particular to a membrane-bound molecule of a reproductive organ or the nervous system of an insect.

In preferred embodiments of the disclosure, the one or more affinity molecule A or a fragment thereof being comprised in the affinity construct of the present disclosure are capable of recognizing, or capable of binding to, or binding to, or being directed to, or being designed to bind specifically to a membrane-bound molecule of the digestive tract of an insect, more preferably to a membrane-bound molecule of the gut or intestine of the insect. Preferably, the membrane-bound molecule is a receptor molecule, more preferably an essential receptor molecule, even more preferably a receptor molecule for a Cry protein. Preferably, the receptor molecule is selected from the group consisting of cadherin protein receptors, aminopeptidase N protein receptors, alkaline phosphatase protein receptors, ABC transporter protein receptors, or any other midgut protein that could serve as a potential receptor, such as chitin synthase B proteins, viral docking proteins, 14-3-3 scaffold proteins or any other membrane bound or membrane-associated molecule.

The at least one affinity molecule A being comprised in the affinity construct of the present disclosure are capable of recognizing, or capable of binding to, or binding to, or being directed to, or being designed to bind to receptors of an inner organ of an insect, preferably of the digestive tract, a reproductive organ or the nervous system, but specifically binds to receptors in the midgut or intestine of an insect or insect pest, as described herein above. As mentioned herein above, the digestive system of insects comprises an alimentary canal or gut, which is divided into three sections: foregut, midgut, and hindgut. The hindgut comprises the intestines, more specifically the Malpighian tubule system, which is where much of the diffusion into the insect's body occurs. In various embodiments, the one more affinity molecule A or a fragment thereof is capable of recognizing, or capable of binding to, or binding to, or being directed to, or being designed to bind to receptors in the midgut or intestine of insect larvae. Any specific structure of an inner organ of an insect, preferably of the digestive tract, a reproductive organ or the nervous system, and more preferably of the midgut or intestine of an insect or an insect larva can be targeted in the context of the present disclosure. In various aspects, this insect-specific structure is a specific structure of the midgut of an insect or an insect larva. In various embodiments, the one or more affinity molecule A or a fragment thereof is capable of recognizing, or capable of binding to, or binding to, or being directed to, or being designed to bind to a midgut membrane protein of an insect or an insect larva. In various embodiments, the midgut membrane protein is an insect or insect larva midgut membrane receptor protein. In various embodiments, the insect or insect larva midgut membrane receptor protein is an insect or insect larva midgut membrane receptor for an insecticidal protein from Bt. In various embodiments of the disclosure, the affinity molecule A or a fragment thereof binds to the apical membrane of insect or insect larvae midgut cells.

In preferred embodiments, the one or more affinity molecule A or a fragment thereof, is capable of recognizing, or capable of binding to, or binding to, or being directed to, or being designed to have been raised against or designed to bind and thus bind specifically to Bt toxin receptor proteins of an insect midgut membrane. In various other embodiments, the affinity molecule A of the present disclosure specifically binds insect-specific structures other than the Bt toxin receptor proteins, which other structures include, but are not limited to, any other molecules embedded in or being located on the insect gut membrane. Examples of such structures are insect midgut membrane proteins or insect midgut membrane-bound proteins or proteins attached to the insect midgut membrane, and include, without being limited thereto, receptor proteins other than the Bacillus thuringiensis (Bt) toxin receptor proteins.

In the context of the present disclosure, insect-specific receptors can be integral part of membranes of the insect gut or can be attached to these membranes via post-transcriptional modifications, including geranyl-phosphatidyl-inositol (GPI) anchors (see FIG. 6 for schematic of how GPI-anchored insect midgut proteins can be used as targets). Such proteins can be identified from transcriptomic (e.g. RNAseq) or proteomic (e.g., Shotgun proteomics, protein sequencing via Mass Spectrometry) analyses of insect midgut proteins, or via proteomic analysis of fractions enriched in midgut membrane proteins. Membrane proteins can be identified using various protein and nucleic acid sequence analysis software tools. Proteins with GPI anchors can be also identified via web-based tools, including big-PI Predictor (GPI Modification Site Prediction, http://mendel.imp.ac.at/sat/gpi/gpi_server.html).

In various embodiments of the disclosure, the one or more affinity molecule A or a fragment thereof is capable of recognizing, or capable of binding to, or binding to, or being directed to, or being designed to bind to a membrane-bound molecule of the intestine of an insect to be targeted by the affinity construct of the present disclosure. In various embodiments of the disclosure, the one or more affinity molecule A or a fragment thereof is capable of recognizing, or capable of binding to, or binding to, or being directed to, or being designed to bind to a membrane-bound molecule of the intestine of an insect larva. In preferred embodiments, the membrane-bound molecule of the intestine of an insect or insect larva is a membrane-bound receptor molecule. More preferably, the membrane-bound receptor molecule of the intestine of an insect or insect larva is a membrane-bound protein (insect or insect larva midgut membrane protein). More specifically, the membrane-bound receptor molecule is a membrane-bound receptor protein. In preferred embodiments, the membrane-bound receptor molecule or membrane-bound receptor protein is a membrane-bound receptor for an insecticidal protein as described herein. More preferably, the membrane-bound receptor for an insecticidal protein is a membrane-bound receptor for a Cry protein.

In various embodiments, the one or more affinity molecule A of the present disclosure is capable of recognizing, or capable of binding to, or binding to, or being directed to, or being designed to specifically to a molecule attached to or being part of a membrane of the target insect or target insect larva. In various embodiments, the membrane of the target insect or target insect larva is a membrane of the midgut of the target insect or target insect larva. In preferred embodiments, the molecule attached to a membrane of the target insect or target insect larva is a receptor attached to a membrane of the midgut of the target insect or target insect larva. More preferably, the receptor attached to a membrane of the midgut of the target insect or target insect larva is a receptor for an insecticidal protein. Even more preferably, the receptor attached to a membrane of the midgut of the target insect or target insect larva is a receptor for a Cry protein.

In various embodiments of the disclosure, the above-mentioned receptor is a cell surface receptor of a cell of the membrane of the midgut of an insect or insect larva. In various embodiments, the above-mentioned specific structure of the midgut of an insect or insect larva serves as or provides one or more epitopes of a cell surface receptor of insect or insect larva midgut membrane cells. In preferred embodiments, the above-mentioned specific structure of the midgut of an insect or insect larva is the extracellular domain of a receptor protein of insect or insect larva midgut membrane cells. More preferably, the above-mentioned specific structure of the midgut of an insect or insect larva serves as or provides one or more epitopes of the extracellular domain of a receptor protein of insect or insect larva midgut membrane cells. In various aspects, the above-mentioned insect or insect larva midgut membrane receptor is a carbohydrate receptor.

In various embodiments, the above-mentioned receptor for a Cry protein to which one or more affinity molecule A or a fragment thereof of the present disclosure is capable of recognizing, or capable of binding to, or binding to, or being directed to, or being designed to bind, is the receptor of a B. thuringiensis Cry protein of any of the 74 major types (classes) of B. thuringiensis delta-endotoxins (i.e., the receptor of any of Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry28, Cry29, Cry30, Cry31, Cry32, Cry33, Cry34, Cry35, Cry36, Cry37, Cry38, Cry39, Cry40, Cry41, Cry42, Cry43, Cry44, Cry45, Cry 46, Cry47, Cry49, Cry50, Cry 51, Cry52, Cry53, Cry54, Cry55, Cry56, Cry57, Cry58, Cry59, Cry60, Cry61, Cry62, Cry63, Cry64, Cry65, Cry66, Cry67, Cry68, Cry69, Cry70, Cry71, Cry72, Cry73 or Cry74). In preferred embodiments of the disclosure, the one or more affinity molecule A or a fragment thereof is capable of recognizing, or capable of binding to, or binding to, or being directed to, or being designed to bind to a receptor of a Cry1 or a Cry3 toxin. More preferably, the one nor more affinity molecule A or a fragment thereof is capable of recognizing, or capable of binding to, or binding to, or being directed to, or being designed to bind to a receptor for a Cry1Ac or a Cry3Aa toxin. In various other embodiments of the disclosure, the affinity molecule A or a fragment thereof is capable of recognizing, or capable of binding to, or binding to, or being directed to, or being designed to bind to a receptor of any one of: Cry1Aa (e.g., Cry1Aa1, Accession #M11250), Cry1Ab (e.g., Cry1Ab1, Accession #M13898), Cry1Ab-like (Accession #AF327924 or #AF327925 or #AF327926), Cry1Ac (e.g., Cry1Ac1, Accession #M11068), Cry1Ad (e.g., Cry1Ad1, Accession #M73250), Cry1Ae (e.g., Cry1Ae1, Accession #M65252), Cry1Af (e.g., Cry1Af1, Accession #U82003), Cry1Ag (e.g., Cry1Ag1, Accession #AF081248), Cry1Ah (e.g., Cry1Ah1, Accession #AF281866), Cry1Ai (e.g., Cry1Ai1, Accession #AY174873), Cry1A-like (Accession #AF327927), Cry1Ba (e.g., Cry1Ba1, Accession #X06711), Cry1Bb (e.g., Cry1Bb1, Accession #L32020), Cry1Bc (e.g., Cry1Bc1, Accession #Z46442), Cry1Bd (e.g., Cry1Bd1, Accession #U70726), Cry1Be (e.g., Cry1Be1, Accession #AF077326), Cry1Bf (e.g., Cry1Bf1, Accession #AX189649), Cry1Bg (e.g., Cry1Bg1, Accession #AY176063), Cry1Ca (e.g., Cry1Ca1, Accession #X07518), Cry1Cb (e.g., Cry1Cb1, Accession #M97880), Cry1Cb-like (Accession #AAX63901), Cry1Da (e.g., Cry1Da1, Accession #X54160), Cry1db (e.g., Cry1db1, Accession #Z22511), Cry1Dc (e.g., Cry1 Dc1, Accession #EF059913), Cry1 Ea (e.g., Cry1 Ea1, Accession #X53985), Cry1 Eb (e.g., Cry1 Eb1, Accession #M73253), Cry1Fa (e.g., Cry1Fa1, Accession #M63897), Cry1Fb (e.g., Cry1Fb1, Accession #Z22512), Cry1Ga (e.g., Cry1Ga1, Accession #Z22510), Cry1Gb (e.g., Cry1Gb1, Accession #U70725), Cry1Gc (Accession #AAQ52381), Cry1Ha (e.g., Cry1Ha1, Accession #Z22513), Cry1Hb (e.g., Cry1Hb1, Accession #U35780), Cry1H-like (Accession #AF182196), Cry1Ia (e.g., Cry1Ia1, Accession #X62821), Cry1Ib (e.g., Cry1Ib1, Accession #U07642), Cry1Ic (e.g., Cry1Ic1, Accession #AF056933), Cry1Id (e.g., Cry1Id1, Accession #AF047579), Cry1Ie (e.g., Cry1Ie1, Accession #AF211190), Cry1If (e.g., Cry1If1, Accession #AAQ52382), Cry1I-like (Accession #190732), Cry1Ja (e.g., Cry1Ja1 (Accession #L32019), Cry1Jb (e.g., Cry1Jb1, Accession #U31527), Cry1Jc (e.g., Cry1Jc1 (Accession #190730), Cry1Jd (e.g., Cry1Jd1 (Accession #AX189651), Cry1Ka (e.g., Cry1Ka1, Accession #U28801), Cry1La (e.g., Cry1La1, Accession #AAS60191), Cry1-like (Accession #190729), Cry2Aa (e.g., Cry2Aa1, Accession #M31738), Cry2Ab (e.g., Cry2Ab1, Accession #M23724), Cry2Ac (e.g., Cry2Ac1, Accession #X57252), Cry2Ad (e.g., Cry2Ad1, Accession #AF200816), Cry2Ae (e.g., Cry2Ae1, Accession #AAQ52362), Cry2Af (e.g., Cry2Af1, Accession #EF439818), Cry2Ag (Accession #ACH91610), Cry2Ah (Accession #EU939453), Cry3Aa (e.g., Cry3Aa1, Accession #M22472), Cry3Ba (e.g., Cry3Ba1, Accession #X17123), Cry3Ca (e.g., Cry3Ca1, Accession #X59797), Cry4Aa (e.g., Cry4Aa1, Accession #00423), Cry4A-like (Accession #DQ078744), Cry4Ba (e.g., Cry4Ba1, Accession #X07423), Cry4Ba-like (Accession #ABC47686), Cry4Ca (e.g., Cry4Ca1, Accession #EU646202), Cry5Aa (e.g., Cry5Aa1, Accession #L07025), Cry5Ab (e.g., Cry5Ab1, Accession #L07026), Cry5Ac (e.g., Cry5Ac1, Accession #134543), Cry5 Ad (e.g., Cry5Ad1, Accession #EF219060), Cry5Ba (e.g., Cry5Ba1, Accession #U19725), Cry6Aa (e.g., Cry6Aa1, Accession #L07022), Cry6Ba (e.g., Cry6Ba1, Accession #L07024), Cry7Aa (e.g., Cry7Aa1, Accession #M64478), Cry7Ab (e.g., Cry7Ab1, Accession #U04367), Cry7Ba (e.g., Cry7Ba1, Accession #ABB70817), Cry7Ca (e.g., Cry7Ca1, Accession #EF486523), Cry8Aa (e.g., Cry8Aa1, Accession #U04364), Cry8Ab (e.g., Cry8Ab1, Accession #EU044830), Cry8Ba (e.g., Cry8Ba1, Accession #U04365), Cry8Bb (e.g., Cry8Bb1, Accession #AX543924), Cry8Bc (e.g., Cry8Bc1, Accession #AX543926), Cry8Ca (e.g., Cry8Ca1, Accession #U04366), Cry8 Da (e.g., Cry8Da1, Accession #AB089299), Cry8db (e.g., Cry8db1, Accession #AB303980), Cry8Ea (e.g., Cry8Ea1, Accession #AY329081), Cry8Fa (e.g., Cry8Fa1, Accession #AY551093), Cry8Ga (e.g., Cry8Ga1, Accession #AY590188), Cry8Ha (e.g., Cry8Ha1, Accession #EF465532), Cry8Ia (e.g., Cry8Ia1, Accession #EU381044), Cry8Ja (e.g., Cry8Ja1, Accession #EU625348), Cry8-like (Accession #ABS53003), Cry9Aa (e.g., Cry9Aa1, Accession #X58120), Cry9Ba (e.g., Cry9Ba1, Accession #X75019), Cry9Bb (e.g., Cry9Bb1, Accession #AY758316), Cry9Ca (e.g., Cry9Ca1, Accession #Z37527), Cry9 Da (e.g., Cry9Da1, Accession #D85560), Cry9db (e.g., Cry9db1, Accession #AY971349), Cry9Ea (e.g., Cry9Ea1, Accession #AB011496), Cry9Eb (e.g., Cry9Eb1, Accession #AX189653), Cry9Ec (e.g., Cry9Ec1, Accession #AF093107), Cry9Ed (e.g., Cry9Ed1, Accession #AY973867), Cry9-like (Accession #AF093107), Cry10Aa (e.g., Cry10Aa1, Accession #M12662), Cry10A-like (Accession #DQ167578), Cry11Aa (e.g., Cry11Aa1, Accession #M31737), Cry11Aa-like (Accession #DQ166531), Cry11 Ba (e.g., Cry11 Ba1, Accession #X86902), Cry11Bb (e.g., Cry11Bb1, Accession #AF017416), Cry12Aa (e.g., Cry12Aa1, Accession #L07027), Cry13Aa (e.g., Cry13Aa1, Accession #L07023), Cry14Aa (e.g., Cry14Aa1, Accession #U13955), Cry15Aa (e.g., Cry15Aa1, Accession #M76442), Cry16Aa (e.g., Cry16Aa1, Accession #X94146), Cry17Aa (e.g., Cry17Aa1, Accession #X99478), Cry18Aa (e.g., Cry18Aa1, Accession #X99049), Cry18Ba (e.g., Cry18Ba1, Accession #AF169250), Cry18Ca (e.g., Cry18Ca1, Accession #AF169251), Cry19Aa (e.g., Cry19Aa1, Accession #Y07603), Cry19Ba (e.g., Cry19Ba1, Accession #D88381), Cry20Aa (e.g., Cry20Aa1, Accession #U82518), Cry21Aa (e.g., Cry21Aa1, Accession #132932), Cry21Ba (e.g., Cry21Ba1, Accession #AB088406), Cry22Aa (e.g., Cry22Aa1, Accession #134547), Cry22Ab (e.g., Cry22Ab1, Accession #AAK50456), Cry22Ba (e.g., Cry22Ba1, Accession #AX472770), Cry23Aa (e.g., Cry23Aa1, Accession #AAF76375), Cry24Aa (e.g., Cry24Aa1, Accession #U88188), Cry24Ba (e.g., Cry24Ba1, Accession #BAD32657), Cry24Ca (e.g., Cry24Ca1, Accession #AM158318), Cry25Aa (e.g., Cry25Aa1, Accession #U88189), Cry26Aa (e.g., Cry26Aa1, Accession #AF122897), Cry27Aa (e.g., Cry27Aa1, Accession #AB023293), Cry28Aa (e.g., Cry28Aa1, Accession #AF132928), Cry29Aa (e.g., Cry29Aa1, Accession #AJ251977), Cry30Aa (e.g., Cry30Aa1, Accession #AJ251978), Cry30Ba (e.g., Cry30Ba1, Accession #BAD00052), Cry30Ca (e.g., Cry30Ca1, Accession #BAD67157), Cry30 Da (e.g., Cry30Da1, Accession #EF095955), Cry30db (e.g., Cry30db1, Accession #BAE80088), Cry30Ea (e.g., Cry30Ea1, Accession #EU503140), Cry30Fa (e.g., Cry30Fa1, Accession #EU751609), Cry30Ga (e.g., Cry30Ga1, Accession #EU882064), Cry31 Aa (e.g., Cry31Aa1, Accession #AB031065), Cry31Ab (e.g., Cry31Ab1, Accession #AB250923), Cry31Ac (e.g., Cry31Ac1, Accession #AB276125), Cry32Aa (e.g., Cry32Aa1, Accession #AY008143), Cry32Ba (e.g., Cry32Ba1, Accession #BAB78601), Cry32Ca (e.g., Cry32Ca1, Accession #BAB78602), Cry32 Da (e.g., Cry32Da1, Accession #BAB78603), Cry33Aa (e.g., Cry33Aa1, Accession #AAL26871), Cry34Aa (e.g., Cry34Aa1, Accession #AAG50341), Cry34Ab (e.g., Cry34Ab1, Accession #AAG41671), Cry34Ac (e.g., Cry34Ac1, Accession #AAG50118), Cry34Ba (e.g., Cry34Ba1, Accession #AAK64565), Cry35Aa (e.g., Cry35Aa1, Accession #AAG50342), Cry35Ab (e.g., Cry35Ab1, Accession #AAG41672), Cry35Ac (e.g., Cry35Ac1, Accession #AAG50117), Cry35Ba (e.g., Cry35Ba1, Accession #AAK64566), Cry36Aa (e.g., Cry36Aa1, Accession #AAK64558), Cry37Aa (e.g., Cry37Aa1, Accession #AAF76376), Cry38Aa (e.g., Cry38Aa1, Accession #AAK64559), Cry39Aa (e.g., Cry39Aa1, Accession #BAB72016), Cry40Aa (e.g., Cry40Aa1, Accession #BAB72018), Cry40Ba (e.g., Cry40Ba1, Accession #BAC77648), Cry40Ca (e.g., Cry40Ca1, Accession #EU381045), Cry40 Da (e.g., Cry40Da1, Accession #EU596478), Cry41Aa (e.g., Cry41Aa1, Accession #AB116649), Cry41Ab (e.g., Cry41Ab1, Accession #AB116651), Cry42Aa (e.g., Cry42Aa1, Accession #AB116652), Cry43Aa (e.g., Cry43Aa1, Accession #AB115422), Cry43Ba (e.g., Cry43Ba1, Accession #AB115422), Cry43-like (Accession #AB115422), Cry44Aa (Accession #BAD08532), Cry45Aa (Accession #BAD22577), Cry46Aa (Accession #BAC79010), Cry46Ab (Accession #BAD35170), Cry47Aa (Accession #AY950229), Cry48Aa (Accession #AJ841948), Cry48Ab (Accession #AM237207), Cry49Aa (Accession #AJ841948), Cry49Ab (e.g., Cry49Ab1, Accession #AM237202), Cry50Aa (e.g., Cry50Aa1, Accession #AB253419), Cry51Aa (e.g., Cry51Aa1, Accession #DQ836184), Cry52Aa (e.g., Cry52Aa1, Accession #EF613489), Cry53Aa (e.g., Cry53Aa1, Accession #EF633476), Cry54Aa (e.g., Cry54Aa1, Accession #EU339367), and Cry55Aa (e.g., Cry55Aa1, Accession #EU121521). In various embodiments of the disclosure, the one or more affinity molecule A or a fragment thereof is capable of recognizing, or capable of binding to, or binding to, or being directed to, or being designed to bind to a receptor of Cry1Ac, Cry1A.105, Cry2Ab2, Cry3Aa or Cry3Bb1.

In other preferred embodiments of the disclosure, the one or more affinity molecule A or a fragment thereof is capable of recognizing, or capable of binding to, or binding to, or being directed to, or being designed to bind to a receptor of the Cyt toxins of B. thuringiensis, preferably to a receptor of the Cyt1 and Cyt2 toxins of B. thuringiensis.

In various embodiments of the disclosure, the one or more affinity molecule A or a fragment thereof is capable of recognizing, or capable of binding to, or binding to, or being directed to, or being designed to bind to a receptor of the Cry toxin that is derived from the B. thuringiensis strain kurstaki (Btk) HD1, which expresses Cry1Aa, Cry1Ab, Cry1Ac and Cry2Aa proteins, or binds to a receptor of the Cry toxin that is derived from B. thuringiensis strain HD73, which produces Cry1Ac (effective in controlling many leaf-feeding lepidopterans that are important crop pests or forest pest defoliators). In various other embodiments of the present disclosure, the one or more affinity molecule A or a fragment thereof is capable of recognizing, or capable of binding to, or binding to, or being directed to, or being designed to bind to a receptor of the Cry toxin that is derived from B. thuringiensis var. aizawai HD137, which produces slightly different Cry toxins such as Cry1Aa, Cry1Ba, Cry1Ca and Cry1 Da (active against lepidopteran larvae that feed on stored grains). In yet other embodiments of the present disclosure, the one or more affinity molecule A or a fragment thereof is capable of recognizing, or capable of binding to, or binding to, or being directed to, or being designed to bind to a receptor of the Cry toxin that is derived from B. thuringiensis var. san diego or B. thuringiensis var. tenebrionis, which produce Cry3Aa toxin and Cry4A, Cry4B, Cry11A and Cyt1Aa toxins (active against coleopteran pests). In still other embodiments of the present disclosure, the one or more affinity molecule A or a fragment thereof is capable of recognizing, or capable of binding to, or binding to, or being directed to, or being designed to bind to a receptor of a Cry toxin showing toxicity against mosquitoes, like Cry1, Cry2, Cry4, Cry11, and Cry29. Thus, in one embodiment of the present disclosure, the one or more affinity molecule A or a fragment thereof is capable of recognizing, or capable of binding to, or binding to, or being directed to, or being designed to bind to a receptor of the Cry toxin that is derived from Bt var israelensis (Bti.), which has been used worldwide for the control of mosquitoes.

In various embodiments of the present disclosure, the one or more affinity molecule A or a fragment thereof is capable of recognizing, or capable of binding to, or binding to, or being directed to, or being designed to bind to a receptor of the B. thuringiensis Cyt1 or Cyt2 toxin. In various other embodiments of the disclosure, the one or more affinity molecule A or a fragment thereof is capable of recognizing, or capable of binding to, or binding to, or being directed to, or being designed to bind to a receptor of the DIG-3 or DIG-11 toxin, which are N-terminal deletions of alpha-helix 1 and/or alpha-helix 2 variants of Cry proteins such as Cry1A described in U.S. Pat. Nos. 8,304,604 and 8,304,605.

In preferred embodiments of the present disclosure, the one or more affinity molecule A or a fragment thereof is capable of recognizing, or capable of binding to, or binding to, or being directed to, or being designed to bind to a receptor of the Cry proteins that belong to the three-domain Cry (3d-Cry) group, which is the largest family of Cry proteins, with members that show toxicity against different insect orders, such as Hymenoptera, Hemiptera, Lepidoptera, Diptera and Coleoptera. In various embodiments of the present disclosure, the one or more affinity molecule A or a fragment thereof is capable of recognizing, or capable of binding to, or binding to, or being directed to, or being designed to bind to the region of a receptor of a 3d-Cry protein, which is involved in recognition of domain II of a 3d-Cry protein. In various embodiments of the present disclosure, the one or more affinity molecule A or a fragment thereof is capable of recognizing, or capable of binding to, or binding to, or being directed to, or being designed to bind to the region of a receptor of a 3d-Cry protein, which is involved in recognition of domain III of a 3d-Cry protein.

In various other embodiments of the present disclosure, the one or more affinity molecule A or a fragment thereof is capable of recognizing, or capable of binding to, or binding to, or being directed to, or being designed to bind to a receptor of insecticidal lipases including, but not limited to, receptors of lipid acyl hydrolases as described in U.S. Pat. No. 7,491,869, and receptors of cholesterol oxidases such as, for example, from Streptomyces.

In other preferred embodiments of the present disclosure, the one or more affinity molecule A or a fragment thereof is capable of recognizing, or capable of binding to, or binding to, or being directed to, or being designed to bind to a receptor of a Vip (vegetative insecticidal protein) toxin from Bacillus thuringiensis. More preferably, the one or more affinity molecule A or a fragment thereof is capable of recognizing, or capable of binding to, or binding to, or being directed to, or being designed to bind to a receptor of a Vip1, Vip2 or Vip3 protein. In various embodiments, the one or more affinity molecule A or a fragment thereof is capable of recognizing, or capable of binding to, or binding to, or being directed to, or being designed to bind to a receptor of a Vip protein from B. thuringiensis. Preferably, the one or more affinity molecule A or a fragment thereof is capable of recognizing, or capable of binding to, or binding to, or being directed to, or being designed to bind to a receptor of a B. thuringiensis Vip1 or a receptor of a Vip2 protein. In various embodiments, the one or more affinity molecule A or a fragment thereof is capable of recognizing, or capable of binding to, or binding to, or being directed to, or being designed to bind to a receptor of a B. thuringiensis Vip3 protein. In various embodiments, the one or more affinity molecule A or a fragment thereof is capable of recognizing, or capable of binding to, or binding to, or being directed to, or being designed to bind to a receptor of a B. thuringiensis Vip3A protein or a receptor of a B. thuringiensis Vip3B protein.

In other embodiments of the present disclosure, the one or more affinity molecule A or a fragment thereof is capable of recognizing, or capable of binding to, or binding to, or being directed to, or being designed to bind to a receptor of a other identified or re-classified insecticidal proteins produced by B. thuringiensis including but not limited to Tpp, Mpp, Gpp, App, Spp, Vpa, Vpb, Mcf, Pra, Prb, Xpp, Mpf (see Table 1 of Crickmore et al. 2020, Journal of Invertebrate Pathology, 107438). One recently identified member of Vpb4 insecticidal protein family Vpb4Da2 is particurly active against western corn rootworm (Yin et al. 2020 PLOS one, 15(11): e0242792).

In various embodiments of the present disclosure, the one or more affinity molecule A or a fragment thereof is capable of recognizing, or capable of binding to, or binding to, or being directed to, or being designed to bind to a receptor of an Mtx protein (mosquitocidal toxin). In various other embodiments of the present disclosure, the one or more affinity molecule A or a fragment thereof is capable of recognizing, or capable of binding to, or binding to, or being directed to, or being designed to bind to a receptor of a Bin protein (binary toxin). In various further embodiments of the present disclosure, one or more the affinity molecule A or a fragment thereof is capable of recognizing, or capable of binding to, or binding to, or being directed to, or being designed to bind to a receptor of a Sip protein (secreted insecticidal toxins).

In various embodiments of the present disclosure, the one or more affinity molecule A or a fragment thereof is capable of recognizing, or capable of binding to, or binding to, or being directed to, or being designed to bind to receptors of toxin complex (TC) proteins, obtainable from organisms such as Xenorhabdus, Photorhabdus and Paenibacillus. In various other embodiments of the present disclosure, the one or more affinity molecule A or a fragment thereof is capable of recognizing, or capable of binding to, or binding to, or being directed to, or being designed to bind to a receptor of spider, snake and scorpion venom proteins.

Methods for identifying receptors of insecticidal proteins are well known in the art (see, Hofmann et. al. (1988, Eur. J. Biochem. 173:85-91; Gill et al. 1995, J. Biol. Chem. 27277-27282) and can be employed to identify and isolate the receptor that recognizes a given insecticidal protein using brush-border membrane vesicles from susceptible insects. Brush-border membrane vesicles (BBMV) of susceptible insects can be prepared according to the protocols listed in the references and separated on SDS-PAGE gel and blotted on a suitable membrane. Labeled insecticidal proteins can be incubated with blotted membrane of BBMV and the insecticidal proteins can be identified with the labeled reporters. Identification of protein band(s) that interact with the insecticidal proteins can be detected by N-terminal amino acid gas phase sequencing or mass spectrometry-based protein identification method (see, Patterson 1998, Current Protocol in Molecular Biology 10(22): 1-24, published by John Wiley & Son Inc). Once the protein is identified, the corresponding gene can be cloned from genomic DNA or cDNA library of the susceptible insects and binding affinity can be measured directly with the insecticidal proteins.

The present disclosure also contemplates affinity molecules A that are capable of recognizing, or capable of binding to, or binding to, or being directed to, or being designed to bind insect-specific structures (such as receptor proteins) in membranes beyond the insect intestine. In various embodiments, targeting of such structures is accomplished by using viral packaging or other packaging means. Relevant insect midgut membranes and feasible receptors sitting in or on such membranes are described in the literature.

The present disclosure also encompasses affinity molecules A that are capable of recognizing, or capable of binding to, or binding to, or being directed to, or being designed to bind insect-specific structures other than the above-mentioned structures, including, but not limited to, protein modifications like, for example, glycosylations, phosphorylations, methylations, acetylations, farnesylations etc., or membrane-lipid modifications like, for example, glycosylations, phosphorylations, specific fatty acids, etc.

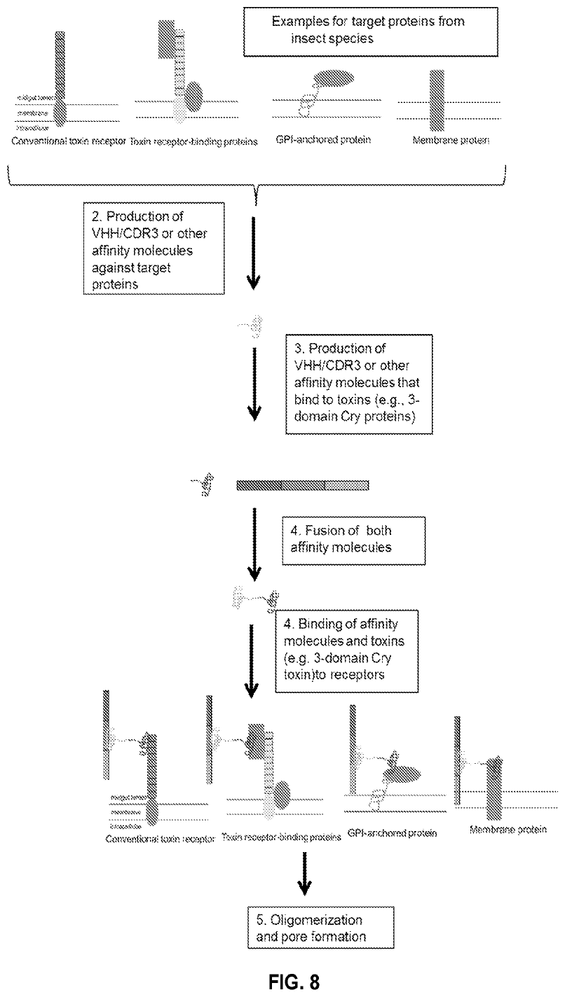

In preferred embodiments of the disclosure, the one or more affinity molecule A or a fragment thereof is capable of recognizing, or capable of binding to, or binding to, or being directed to, or being designed to bind to any one of: cadherin proteins or epitopes thereof; aminopeptidase N proteins or epitopes thereof; alkaline phosphatase proteins or epitopes thereof; ABC transporter proteins or epitopes thereof; 270 kDa glycoconjugate proteins or epitopes thereof; a 250 kDa protein named P252 or epitopes thereof; or any other insect receptor protein that might naturally bind to insecticidal proteins such as Cry proteins. In further embodiments, the one or more affinity molecule A or a fragment thereof is capable of recognizing, or capable of binding to, or binding to, or being directed to, or being designed to bind to insect-structures that do not yet serve as receptors such as, for example, membrane proteins or proteins that are associated to the membrane or interact with membrane proteins, or to modifications of such proteins (e.g., glycosyl, lipoyl, sumoyl, ubiquitin, phosphate residues, see FIG. 8).

In preferred embodiments of the present disclosure, the one or more affinity molecule A or a fragment thereof is capable of recognizing, or capable of binding to, or binding to, or being directed to, or being designed to bind to the Spodoptera frugiperda cadherin receptor. Preferably, the one or more affinity molecule A or a fragment is capable of recognizing, or capable of binding to, or binding to, or being directed to, or being designed to bind to the extracellular domain of the Spodoptera frugiperda cadherin. The nucleotide and amino acid sequence of the Spodoptera frugiperda cadherin is shown in (SEQ ID NOS. 1 and 2) respectively, with amino acids 1-1610 representing the extracellular domain. Additionally, the Cyt toxin binding region of Spodoptera frugiperda cadherin is provided in SEQ ID. NOS. 42.

In other preferred embodiments of the present disclosure, the one or more affinity molecule A or a fragment thereof is capable of recognizing, or capable of binding to, or binding to, or being directed to, or being designed to bind to the Helicoverpa armigera cadherin receptor. Preferably, the one or more affinity molecule A or a fragment thereof is capable of recognizing, or capable of binding to, or binding to, or being directed to, or being designed to bind to the extracellular domain of the Helicoverpa armigera cadherin. The nucleotide and amino acid sequence of the Helicoverpa armigera cadherin is presented in SEQ ID. NOS. 3 and 4, with amino acids 1-1583 representing the extracellular domain. In other preferred embodiments of the present disclosure, the one or more affinity molecule A or a fragment thereof is capable of recognizing, or capable of binding to, or binding to, or being directed to, or being designed to thus bind to the Diabrotica virgifera virgifera cadherin receptor. Preferably, the one or more affinity molecule A or a fragment thereof is capable of recognizing, or capable of binding to, or binding to, or being directed to, or being designed to bind to the extracellular domain of the Diabrotica virgifera virgifera cadherin. The nucleotide and amino acid sequence of the Diabrotica virgifera virgifera cadherin is presented in SEQ ID. NOS. 5 and 6, with amino acids 1-1572 representing the extracellular domain. In other preferred embodiments of the present disclosure, the one or more affinity molecule A or a fragment thereof is capable of recognizing, or capable of binding to, or binding to, or being directed to, or being designed to bind to the Heliothis virescens cadherin receptor. Preferably, the one or more affinity molecule A or a fragment thereof is capable of recognizing, or capable of binding to, or binding to, or being directed to, or being designed to bind to the extracellular domain of the Heliothis virescens cadherin. The nucleotide and amino acid sequence of the Heliothis virescens cadherin is presented in SEQ ID. NOS. 7 and 8 respectively with amino acids 1-1583 representing the extracellular domain. In other preferred embodiments, the one or more affinity molecule A or a fragment thereof is capable of recognizing, or capable of binding to, or binding to, or being directed to, or being designed to bind to the Helicoverpa armigera chitin synthase B receptor. Preferably, the one or more affinity molecule A or a fragment thereof is capable of recognizing, or capable of binding to, or binding to, or being directed to, or being designed to bind to the extracellular domain of the Helicoverpa armigera chitin synthase B. The nucleotide and amino acid sequence of the Helicoverpa armigera chitin synthase B is presented in SEQ ID. NOS. 9 and 10 respectively, with amino acids 1048-1242 and 1324-1528 representing the extracellular domain. In still other preferred embodiments of the present disclosure, the one or more affinity molecule A or a fragment thereof is capable of recognizing, or capable of binding to, or binding to, or being directed to, or being designed to bind to the Spodoptera frugiperda chitin synthase B receptor. Preferably, the one or more affinity molecule A or a fragment thereof is capable of recognizing, or capable of binding to, or binding to, or being directed to, or being designed to bind to the extracellular domain of the Spodoptera frugiperda chitin synthase B. The nucleotide and amino acid sequence of the Spodoptera frugiperda chitin synthase B is presented in SEQ ID. NOS. 11 and 12 respectively, with amino acids 1048-1242 and 1321-1523 representing the extracellular domain.

In even other preferred embodiments of the present disclosure, the one or more affinity molecule A or a fragment thereof is capable of recognizing, or capable of binding to, or binding to, or being directed to, or being designed to bind to the Helicoverpa armigera aminopeptidase N. Preferably, the one or more affinity molecule A or a fragment thereof is capable of recognizing, or capable of binding to, or binding to, or being directed to, or being designed to bind to the extracellular domain of the Helicoverpa armigera aminopeptidase N. The nucleic acid sequence of the Helicoverpa armigera aminopeptidase N is provided as SEQ ID NOS. 13 and 14 respectively with amino acids 126-190 representing the extracellular domain.

In other preferred embodiments of the present disclosure, the one or more affinity molecule A or a fragment thereof is capable of recognizing, or capable of binding to, or binding to, or being directed to, or being designed to bind to the Heliothis virescens aminopeptidase N. Preferably, the one or more affinity molecule A or a fragment thereof is capable of recognizing, or capable of binding to, or binding to, or being directed to, or being designed to bind to the extracellular domain of the Heliothis virescens Aminopeptidase N. The nucleic acid and amino acid sequence of the Heliothis virescens Aminopeptidase N is provided as SEQ ID NOS. 15 and 16 respectively with amino acids 126-185 representing the extracellular domain.

In other preferred embodiments of the present disclosure, the one or more affinity molecule A or a fragment thereof is capable of recognizing, or capable of binding to, or binding to, or being directed to, or being designed to bind to the Helicoverpa armigera alkaline phosphatase receptor. Preferably, the one or more affinity molecule A or a fragment thereof is capable of recognizing, or capable of binding to, or binding to, or being directed to, or being designed to bind to the extracellular domain of the Helicoverpa armigera alkaline phosphatase. The nucleic acid and amino acid sequence of the Helicoverpa armigera alkaline phosphatase is provided as SEQ ID NOS. 17 and 18 respectively, with amino acids 192-446 representing the extracellular domain.

In still other preferred embodiments of the present disclosure, the one or more affinity molecule A or a fragment thereof is capable of recognizing, or capable of binding to, or binding to, or being directed to, or being designed to bind to the Heliothis virescens alkaline phosphatase receptor. Preferably, the one or more affinity molecule A or a fragment thereof is capable of recognizing, or capable of binding to, or binding to, or being directed to, or being designed to bind to the extracellular domain of the Heliothis virescens alkaline phosphatase. The nucleic acid sequence and amino acid sequence of the Heliothis virescens alkaline phosphatase is provided as SEQ ID NOS. 19 and 20 respectively, with amino acids 196-450 representing the extracellular domain. In still other preferred embodiments of the present disclosure, the one or more affinity molecule A or a fragment thereof is capable of recognizing, or capable of binding to, or binding to, or being directed to, or being designed to bind to the Spodoptera frugiperda alkaline phosphatase receptor. Preferably, the one or more affinity molecule A or a fragment thereof is capable of recognizing, or capable of binding to, or binding to, or being directed to, or being designed to bind to the extracellular domain of the Spodoptera frugiperda alkaline phosphatase. The nucleic acid and amino acid sequence of the Spodoptera frugiperda alkaline phosphatase is provided as SEQ ID NOS. 21 and 22 respectively, with amino acids 191-451 representing the extracellular domain. In other preferred embodiments of the present disclosure, the one or more affinity molecule A or a fragment thereof is capable of recognizing, or capable of binding to, or binding to, or being directed to, or being designed to bind to the Heliothis virescens ABCC2 receptor. Preferably, the one or more affinity molecule A or a fragment thereof is capable of recognizing, or capable of binding to, or binding to, or being directed to, or being designed to bind to the extracellular domain of the Heliothis virescens ABCC2 receptor. The nucleic acid and amino acid sequence of the Heliothis virescens ABCC2 is provided as SEQ ID NOS. 23 and 24 respectively, with amino acids 1-1339 representing the extracellular domain.

In other preferred embodiments of the present disclosure, the one or more affinity molecule A or a fragment thereof is capable of recognizing, or capable of binding to, or binding to, or being directed to, or being designed to bind to the Helicoverpa armigera ABCC2 receptor. Preferably, the one or more affinity molecule A or a fragment thereof is capable of recognizing, or capable of binding to, or binding to, or being directed to, or being designed to bind to the extracellular domain of the Helicoverpa armigera ABCC2 receptor. The nucleic acid and amino acid sequence of the Helicoverpa armigera ABCC2 is provided as SEQ ID NOS. 25 and 26 respectively, with amino acids 1-1338 representing the extracellular domain.

In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence of the extracellular domain of the Spodoptera frugiperda cadherin (Seq ID NOS. 2), with amino acids from 1-1610 representing the extracellular domain. In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence of the extracellular domain of the Helicoverpa armigera cadherin (Seq ID NOS. 4), with amino acids from 1-1583 representing the extracellular domain. In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence of the extracellular domain of the Diabrotica virgifera virgifera cadherin (Seq ID NOS. 6), with amino acids from 1-1572 representing the extracellular domain. In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence of the extracellular domain of the Heliothis virescens cadherin (Seq ID NOS. 8), with amino acids from 1-1583 representing the extracellular domain.

In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence of the extracellular domain of the Helicoverpa armigera chitin synthase B (Seq ID NOS. 10), with amino acids from with amino acids 1048-1242 and 1324-1528 representing the extracellular domain. In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence of the extracellular domain of the Spodoptera frugiperda chitin synthase B (Seq ID NOS. 12), with amino acids from 1048-1242 and 1321-1523 representing the extracellular domain.

In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence encoded by the nucleotide sequence of the extracellular domain of the Helicoverpa armigera aminopeptidase N (Seq ID NOS. 14), with amino acids from 126-190 representing the extracellular domain. In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence encoded by the nucleotide sequence of the extracellular domain of the Heliothis virescens Aminopeptidase N (Seq ID NOS. 16), with amino acids from 126-185 representing the extracellular domain. In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence encoded by the nucleotide sequence of the extracellular domain of the Helicoverpa armigera alkaline phosphatase (Seq ID NOS. 18), with amino acids from 192-446 representing the extracellular domain.

In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence encoded by the nucleotide sequence of the extracellular domain of the Heliothis virescens alkaline phosphatase (Seq ID NOS. 20), with amino acids from 196-450 representing the extracellular domain.

In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence encoded by the nucleotide sequence of the extracellular domain of the Spodoptera frugiperda alkaline phosphatase (Seq ID NOS. 22), with amino acids from 191-451 representing the extracellular domain.

In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence encoded by the nucleotide sequence of the extracellular domain of the Heliothis virescens ABCC2 (Seq ID NOS. 24), with amino acids from 1-1339 representing the extracellular domain.

In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence encoded by the nucleotide sequence of the extracellular domain of the Helicoverpa armigera ABCC2 (Seq ID NOS. 26), with amino acids from 1-1338 representing the extracellular domain.

In various embodiments of the disclosure, the one or more affinity molecule or a fragment thereof comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence of one of the two V_{H}H domains (Seq ID NOS. 29), separated by the linker sequence GGGSGGGG and individual domain provided Seq ID NOS. 28.

Further preferred embodiments with regard to the one or more affinity molecule A or a fragment thereof are affinity molecules or a fragment thereof that is/are capable of recognizing, or capable of binding to, or being directed to, or being designed to bind the antigen of the polypeptide selected from the group consisting of the Spodoptera frugiperda sodium-dependent nutrient amino acid transporter 1-like protein (SEQ ID NOS. 30 (antigen), 31 (full-length)), the Spodoptera frugiperda V-ATPase subunit a protein (SEQ ID NOS. 32 (antigen), 33 (full-length)), the Spodoptera frugiperda Cry1Fa domain II protein (SEQ ID NOS. 34), the Spodoptera frugiperda cadherin (SEQ ID NOS. 35 antigen, Seq. ID NOS. 2 (full-length)), the Spodoptera frugiperda venom dipeptidyl peptidase 4-like isoform X1 protein (SEQ ID NOS. 37 (antigen), 38 (full-length) or the Spodoptera frugiperda peptide-transporter family 1 isoform X1 protein (SEQ ID NOS. 39 (antigen), 40 (full-length).

In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence encoded by the nucleotide sequence of the extracellular domain of the Spodoptera frugiperda sodium-dependent nutrient amino acid transporter 1-like protein. The extracellular domain (antigen) and full-length sequences are provided as SEQ ID NOS. 30 and 31 respectively.

In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence encoded by the nucleotide sequence of the extracellular domain of the Spodoptera frugiperda V-ATPase subunit protein. The extracellular domain and full-length sequences are provided as SEQ ID NOS. 32 (antigen) and 33 respectively.

In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence encoded by the nucleotide sequence of the extracellular domain of the Spodoptera frugiperda Cry1Fa domain II protein. The full-length sequence is provided as SEQ ID NOS. 34, with the extracellular domain encompassing the entire sequence.

In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence encoded by the nucleotide sequence of the extracellular domain of the Spodoptera frugiperda cadherin protein. The extracellular domain and full-length sequences are provided as SEQ ID NOS. 35 (antigen) and 2 respectively.

In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence encoded by the nucleotide sequence of the extracellular domain of the Spodoptera frugiperda venom dipeptidyl peptidase 4-like isoform X1 protein. The extracellular domain and full-length sequences are provided as SEQ ID NOS. 37 (antigen) and 38 respectively.

In various embodiments of the disclosure, the one or more affinity molecule A or a fragment thereof is capable of recognizing, or capable of binding to, or binding to, or being directed to, or being designed to bind to a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence encoded by the nucleotide sequence of the extracellular domain of the Spodoptera frugiperda peptide-transporter family 1 isoform X1 protein. The extracellular domain and full-length sequences are provided as SEQ ID NOS. 39 (antigen) and 40 respectively.

### Insecticidal Proteins

In various embodiments of the disclosure, the one or more affinity molecule B or a fragment thereof being comprised in the affinity construct of the present disclosure is capable of binding to, or binding to, or being directed to, or being designed to bind specifically to one or more proteins that have insecticidal activity against an insect pest.

The insecticidal protein (toxin) against which the above one or more affinity molecule B or a fragment thereof is capable of binding to, or binding to, or being directed to, or being designed to bind exerts its biological activity by contact of the insecticidal protein via the one or more affinity molecule B or a fragment thereof being comprised in the affinity construct of the present invention with a target (receptor) molecule of an inner organ of an insect, preferably of the digestive tract of an insect, a reproductive organ or the nervous system, more preferably of the gut or intestine of an insect.

In various embodiments, the insecticidal protein exerts its biological activity by contact of the protein with an intestine molecule of the insect via the one or more affinity molecule B or a fragment thereof being comprised in the affinity construct of the present invention. This molecule of the intestine generally is a receptor protein. The term insecticidal protein not only includes insecticidal proteins that are active without further processing, but also precursors in an inactive form, which may be activated by inside factors. For example, the insecticidal protein may be a protoxin crystal, which is cleaved inside by a protease so as to provide the toxic monomeric Cry toxin. In case the insecticidal protein is a protoxin, then the affinity molecule B, is directed to or designed to bind to domain(s) in the protoxin that are removed during later protease cleavage of the protoxin so that the affinity-binding of the one or more affinity molecule B or a fragment thereof to the now activated insecticidal protein is maintained.

The activated toxin goes through a complex sequence of binding events including different Cry-binding proteins of the insect gut, finally leading to membrane insertion and pore formation. Cry toxins form pores in the apical membrane of larvae midgut cells, destroying the midgut cells and killing the larvae. Consequently, the activity of insecticidal proteins results in morphological changes of midgut cells after intoxication with the protein toxin.

The interaction of insecticidal proteins with different proteins present in insect midgut cells is a complex process, which involves multiple membrane proteins. The first binding interaction of (activated) insecticidal proteins with membrane proteins serves to concentrate the activated toxin protein in the microvilli membrane of the midgut cells, where the toxin proteins are then able to bind to receptor proteins, which is necessary to trigger the formation of toxin oligomer structures. This process of oligomerization of the toxin proteins finally provides for the formation of the toxin pores, which are essential for the mode of action of the toxins. Mutations in residues of the membrane or receptor proteins of the midgut cells result in loss of toxicity to insects. Such mutations show altered oligomerization or membrane insertion, severely affecting pore formation.

Surprisingly, the novel affinity constructs of the present disclosure overcome the drawbacks of insect resistance that is caused by mutation of membrane and receptor proteins. Further, the novel affinity constructs concentrate the insecticidal protein in the environment in which the insecticidal protein needs to act, e.g. in the microvilli membrane of the midgut cells, and thereby support the process of toxin oligomerization. This provides for an improvement in insecticidal activity of the toxin protein as less insecticidal protein is required for achieving the insecticidal activity. The effects can also be observed with insecticidal compositions of the disclosure comprising a novel affinity construct of the disclosure and an insecticidal protein, wherein the insecticidal protein corresponds to the insecticidal protein the at least one affinity molecule B of the novel affinity construct is capable of binding to, or binding to, or being directed to, or being designed to bind to.

Further, the novel ways described herein improve targeting of insecticidal agents to the insect. Targeting of novel receptors in an insect pest is facilitated to which a certain insect toxin naturally does not bind (e.g., to restore functionality of a given toxin whose natural receptor has changed due to mutation and is not binding anymore the toxin), and/or “arming” of insecticidal proteins is now possible that formerly are not active in a certain insect species due to the fact that its natural receptor is missing. Targeting theses toxins to other receptors in such an insect renders that toxin toxic for the insect. Also surprising is that the novel affinity constructs even work without the affinity molecule A or a fragment thereof being directed to specific membrane proteins, which are the natural target proteins of the insecticidal proteins. The at least one affinity molecule A of the novel affinity construct of the present disclosure may be capable of recognizing, or capable of binding to, or binding to, or being directed to, or being designed to bind to any membrane or receptor protein of the midgut cells.

An alternative way also encompassed herein to address the resistance of insect pests to insecticidal proteins caused by mutations in the receptor proteins is to apply to or express in the plant wild type (i.e., without the mutations conferring resistance against an insecticidal protein) receptor proteins as expressed in the gut of susceptible insects. Upon uptake by the insect these wild type receptor proteins insert themselves into the insect gut either in addition to the mutated receptor proteins or by replacing them. Either way, the presence of wild type receptor proteins allows the insecticidal protein to bind, to insert into the membrane, to form a pore and eventually to kill the insect.

The terms “insecticidal protein” or “insecticidal protein toxin” are intended to encompass proteins (or polypeptides encoding these proteins) the at least one affinity molecule B or a fragment thereof disclosed herein and being comprised in the affinity construct disclosed herein is capable of binding to, or binding to, or being directed to, or being designed to bind and that have toxic activity against one or more insecticidal pests, including, but not limited to, members of the orders Isoptera, Blattodea, Orthoptera, Phthiraptera, Thysanoptera, Hymenoptera, Siphonaptera, Lepidoptera, Diptera, Hemiptera and Coleoptera, or proteins or polypeptides having homology to such an insecticidal or toxic protein. The terms “insecticidal protein toxin” and “insecticidal protein” may be used herein interchangeably.

Referring to the affinity molecule B of the novel affinity construct that is capable of binding, or is binding, or is directed to, or is designed to bind an insecticidal protein (toxin) is intended to mean that the affinity molecule B is capable of binding to, or is binding to, or is directed to, or is designed to bind to a protein or polypeptide that has toxic activity against one or more insecticidal pests, including, but not limited to, members of the orders Isoptera, Blattodea, Orthoptera, Phthiraptera, Thysanoptera, Hymenoptera, Siphonaptera, Lepidoptera, Diptera, Hemiptera and Coleoptera, or proteins or polypeptides having homology to such an insecticidal or toxic protein.

In various embodiments of the present disclosure, the insecticidal protein being part of the novel composition comprising a affinity construct of the disclosure and an insecticidal protein is an insecticidal toxin that is specifically toxic to an insect order of any one of Isoptera, Blattodea, Orthoptera, Phthiraptera, Thysanoptera, Siphonaptera, Lepidoptera, Coleoptera, Hymenoptera, Hemiptera and Diptera. In various embodiments of the present disclosure, the insecticidal protein is an insecticidal toxin that is specifically toxic to an insect family of any one of Crambidae, Noctuidae, Pyralidae, Chrysomelidae, Dynastidae, Elateridae, Melolonthinae, Curcolionidae, Scarabaeidae, Erebidae, Coccinellidae, Mebidae, or Lamiinae. In various embodiments of the present disclosure, the insecticidal protein has insecticidal activity against an insect pest of the order Lepidoptera, including, but not limited to, Ostrinia nubilalis (Europen Corn Borer), Diatraea grandiosella (South Western Corn Borer), Helicoverpa zea (Corn Earworm), Agrotis ipsilon (Black Cutworm), Agrotis subterranea (Granulate Cutworm), Agrotis malefida (Palesided Cutworm), Spodoptera frugiperda (Fall Army worm), Spodoptera eridania (Southern Armyworm), Spodoptera albula (Gray-Streaked Armyworm), Spodoptera cosmioides, Spodoptera ornithogalli, Spodoptera exigua (Beet Cutworm), Helicoverpa armigera (Cotton Bollworm), Helicoverpa zea (Corn Earworm), Heliothis virescens (Tobacco budworm), Diatraea saccharalis (SugarCane Borer), Diatraea grandiosella (South Western Corn Borer), Elasmopalpus lignosellus (Lesser CornStalk Borer), Striacosta albicosta (Western bean cutworm), Chrysodeixis includens (Soybean looper), Pseudaletia sequax (Wheat armyworm), Porosagrotis gypaetina, Euxoa bilitura (Potato Cutworm), Pseudaletia unipuncta (True armyworm), Anticarsia gemmatalis (Velvetbean caterpillar), Plathypena scabra (Green cloverworm), Elasmopalpus lignosellus (Lesser CornStalk Borer), Chrysodeixis includens (Soybean looper), Trichoplusia ni (Cabbage Looper) and Peridroma saucia (Variegated Cutworm). In various embodiments of the present disclosure, the insecticidal protein has insecticidal activity against an insect pest of the order Coleopoptera including, but not limited to Diabrotica virgifera virgifera (Western Corn Rootworm), Diabrotica barberi (Northern Corn Rootworm), Diabrotica speciosa, Diloboderus abderus, Phyllophaga spp (Scarab beetles), Listronotus spp. (Argentine stem weevil), Cerotoma arcuatus, Popillia japonica (Japanese beetle), Colaspis brunnea (Grape colaspis), Cerutoma trifurcata (Bean Leaf Beetle), Epilachna varivestis (Mexican bean beetle), Diabrotica undecimpunctata howardi (Spotted cucumber beetle), Epicauta pestifera (Blister beetles), Popillia japonica (Japanese beetle), Colaspis brunnea (Grape colaspis), Dectes texanus texanus (Soybean stem borer), and Anthonomous grandis (Boll weevil). In various embodiments of the present disclosure, the insecticidal protein has insecticidal activity against insect pests including, but not limited to Oscinella frit (Fruit Fly), Myzus persicae (Green Peach Aphid), Rhopalosiphum maidis (Corn Leaf Aphid) and Rhopalosiphum padi (Bird Cherry-Oat Aphid).

The insecticidal protein that is part of the novel composition comprising an affinity construct of the disclosure together with one or more insecticidal protein(s) can be any protein that is harmful to an insect and that the at least one affinity molecule B being part of the affinity construct is capable of binding to, or is binding to, or is being directed to, or is being designed to bind. Insecticidal proteins have been isolated from organisms including, e.g., Bacillus sp. and Pseudomonas sp. In various embodiments of the disclosure, the insecticidal protein is derived from Bacillus sp. or Pseudomonas sp. In various embodiments of the disclosure, the insecticidal protein is derived from Bacillus thuringiensis. In various embodiments of the disclosure, the insecticidal protein is an insecticidal crystal protein (ICP). Such ICPs are protein crystals formed during sporulation in some Bacillus thuringiensis strains (Bacillus thuringiensis produces proteins that aggregate to form crystals). The crystal proteins are toxic to very specific insect pest species. The crystal proteins bind specifically to certain receptors in the insect's intestine or midgut. The Bt ICPs are also known as Bt delta-endotoxins. Delta-endotoxins, which have been isolated from Bacillus thuringiensis, include, but are not limited to, the Cry1 to Cry74 classes of delta-endotoxin genes and the Bt cytolytic Cyt genes, in particular Cyt1 and Cyt2. Cyt proteins are toxins mostly found in Bacillus thuringiensis strains active against Diptera, although a few exceptions of Cyt proteins active against Coleopteran larvae have been documented. These proteins can synergize Cry activities against mosquitos and black flies.

The insecticidal activity of Cry proteins is well known to one skilled in the art (for review, see, e.g., www.btnomenclature.info, “Insect Midgut and Insecticidal Proteins”, Vol 47, Advances in Insect Physiology edited by Tarlochan S. Dhadialla, Sarjeet Gill, August 2014: chapter 2: “Diversity of Bacillus thuringiensis Crystal Toxins and Mechansims of Action”: pages 39-87; Academic Press, UK., ISBN: 978-0-12-800197-4, or Pardo-Lopez et al. 2013, FEMS Microbiol Rev 37, 3-22).

Cry proteins are specifically toxic to different insect orders such as Lepidoptera, Coleoptera, Hymenoptera and Diptera. In preferred embodiments of the present disclosure, the insecticidal protein is a Bt Cry protein. In more preferred embodiments of the present disclosure, the insecticidal protein is a Bt Cry toxin that is specifically toxic to an insect order of any of Lepidoptera, Coleoptera, Hymenoptera and Diptera. In other preferred embodiments of the present disclosure, the insecticidal protein is a Bt Cry toxin that is specifically toxic to an insect order of any of Isoptera, Blattodea, Orthoptera, Phthiraptera, Thysanoptera, Siphonaptera, Lepidoptera, Coleoptera, Hymenoptera, Hemiptera and Diptera.

In various embodiments of the disclosure, the insecticidal protein that is part of the novel composition comprising an affinity construct of the disclosure together with one or more insecticidal protein(s) can be any protein that is harmful to an insect and that the at least one affinity molecule B being part of the affinity construct is capable of binding to, or is binding to, or is being directed to, or is being designed to bind, is a protoxin crystal, which is cleaved inside by a protease so as to yield a monomeric Cry toxin. In various embodiments of the disclosure, said insecticidal protein is the monomeric form of an insecticidal toxin. In various embodiments of the disclosure, the insecticidal protein is a monomeric Cry toxin. In various embodiments of the disclosure, the insecticidal protein is the multimeric form of an insecticidal toxin. In various embodiments of the disclosure, the insecticidal protein is a multimeric Cry toxin comprising, e.g., up to, but not limited to, four subunits of a Cry protein.

In preferred embodiments of the present disclosure, the insecticidal protein is a Bt Cry protein. In more preferred embodiments of the present disclosure, the insecticidal protein is a Bt Cry protein of any of the currently 74 major types (classes) of Bt delta-endotoxins (i.e., any of a Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry28, Cry29, Cry30, Cry31, Cry32, Cry33, Cry34, Cry35, Cry36, Cry37, Cry38, Cry39, Cry40, Cry41, Cry42, Cry43, Cry44, Cry45, Cry 46, Cry47, Cry49, Cry50, Cry 51, Cry52, Cry53, Cry54, Cry55, Cry56, Cry57, Cry58, Cry59, Cry60, Cry61, Cry62, Cry63, Cry64, Cry65, Cry66, Cry67, Cry68, Cry69, Cry70, Cry71, Cry72, Cry73 or Cry74 delta-endotoxin). In various embodiments, the insecticidal protein is a Cry1 or a Cry3 protein, more specifically a Cry1 or a Cry3 Bt delta-endotoxin. The Cry1 (Bt delta-) endotoxin is considered to be particularly effective against Lepidoptera, and the Cry3 (Bt delta-) endotoxin is considered to be particularly effective against Coleoptera. The Cry1 or a Cry3 Bt delta-endotoxins are therefore preferred insecticidal proteins in the context of the present invention with Lepidoptera and Coleoptera, respectively, accordingly being preferred insect pest targets according to the present disclosure.

In more preferred embodiments, the insecticidal protein is a Cry1Ac or a Cry3Aa Bt delta-endotoxin. In a preferred embodiment, the insecticidal protein is a Cry1Ac Bt delta-endotoxin. In various other embodiments, the insecticidal protein is a Bt delta-endotoxin of any one of: Cry1Aa (e.g., Cry1Aa1, Accession #M11250), Cry1Ab (e.g., Cry1Ab1, Accession #M13898), Cry1Ab-like (Accession #AF327924 or #AF327925 or #AF327926), Cry1Ac (e.g., Cry1Ac1, Accession #M11068), Cry1Ad (e.g., Cry1Ad1, Accession #M73250), Cry1Ae (e.g., Cry1Ae1, Accession #M65252), Cry1Af (e.g., Cry1Af1, Accession #U82003), Cry1Ag (e.g., Cry1Ag1, Accession #AF081248), Cry1Ah (e.g., Cry1Ah1, Accession #AF281866), Cry1Ai (e.g., Cry1Ai1, Accession #AY174873), Cry1A-like (Accession #AF327927), Cry1Ba (e.g., Cry1Ba1, Accession #X06711), Cry1Bb (e.g., Cry1Bb1, Accession #L32020), Cry1Bc (e.g., Cry1Bc1, Accession #Z46442), Cry1Bd (e.g., Cry1Bd1, Accession #U70726), Cry1Be (e.g., Cry1Be1, Accession #AF077326), Cry1Bf (e.g., Cry1Bf1, Accession #AX189649), Cry1Bg (e.g., Cry1Bg1, Accession #AY176063), Cry1Ca (e.g., Cry1Ca1, Accession #X07518), Cry1Cb (e.g., Cry1Cb1, Accession #M97880), Cry1Cb-like (Accession #AAX63901), Cry1 Da (e.g., Cry1Da1, Accession #X54160), Cry1db (e.g., Cry1db1, Accession #Z22511), Cry1Dc (e.g., Cry1 Dc1, Accession #EF059913), Cry1 Ea (e.g., Cry1 Ea1, Accession #X53985), Cry1 Eb (e.g., Cry1 Eb1, Accession #M73253), Cry1Fa (e.g., Cry1Fa1, Accession #M63897), Cry1Fb (e.g., Cry1Fb1, Accession #Z22512), Cry1Ga (e.g., Cry1Ga1, Accession #Z22510), Cry1Gb (e.g., Cry1Gb1, Accession #U70725), Cry1Gc (Accession #AAQ52381), Cry1Ha (e.g., Cry1Ha1, Accession #Z22513), Cry1Hb (e.g., Cry1Hb1, Accession #U35780), Cry1H-like (Accession #AF182196), Cry1Ia (e.g., Cry1Ia1, Accession #X62821), Cry1Ib (e.g., Cry1Ib1, Accession #U07642), Cry1Ic (e.g., Cry1Ic1, Accession #AF056933), Cry1Id (e.g., Cry1Id1, Accession #AF047579), Cry1Ie (e.g., CryIle1, Accession #AF211190), Cry1If (e.g., Cry1If1, Accession #AAQ52382), Cry1I-like (Accession #190732), Cry1Ja (e.g., Cry1Ja1 (Accession #L32019), Cry1Jb (e.g., Cry1Jb1, Accession #U31527), Cry1Jc (e.g., Cry1Jc1 (Accession #190730), Cry1Jd (e.g., Cry1Jd1 (Accession #AX189651), Cry1Ka (e.g., Cry1Ka1, Accession #U28801), Cry1La (e.g., Cry1La1, Accession #AAS60191), Cry1-like (Accession #190729), Cry2Aa (e.g., Cry2Aa1, Accession #M31738), Cry2Ab (e.g., Cry2Ab1, Accession #M23724), Cry2Ac (e.g., Cry2Ac1, Accession #X57252), Cry2Ad (e.g., Cry2Ad1, Accession #AF200816), Cry2Ae (e.g., Cry2Ae1, Accession #AAQ52362), Cry2Af (e.g., Cry2Af1, Accession #EF439818), Cry2Ag (Accession #ACH91610), Cry2Ah (Accession #EU939453), Cry3Aa (e.g., Cry3Aa1, Accession #M22472), Cry3Ba (e.g., Cry3Ba1, Accession #X17123), Cry3Ca (e.g., Cry3Ca1, Accession #X59797), Cry4Aa (e.g., Cry4Aa1, Accession #00423), Cry4A-like (Accession #DQ078744), Cry4Ba (e.g., Cry4Ba1, Accession #X07423), Cry4Ba-like (Accession #ABC47686), Cry4Ca (e.g., Cry4Ca1, Accession #EU646202), Cry5Aa (e.g., Cry5Aa1, Accession #L07025), Cry5Ab (e.g., Cry5Ab1, Accession #L07026), Cry5Ac (e.g., Cry5Ac1, Accession #134543), Cry5 Ad (e.g., Cry5Ad1, Accession #EF219060), CrySBa (e.g., Cry5Ba1, Accession #U19725), Cry6Aa (e.g., Cry6Aa1, Accession #L07022), Cry6Ba (e.g., Cry6Ba1, Accession #L07024), Cry7Aa (e.g., Cry7Aa1, Accession #M64478), Cry7Ab (e.g., Cry7Ab1, Accession #U04367), Cry7Ba (e.g., Cry7Ba1, Accession #ABB70817), Cry7Ca (e.g., Cry7Ca1, Accession #EF486523), Cry8Aa (e.g., Cry8Aa1, Accession #U04364), Cry8Ab (e.g., Cry8Ab1, Accession #EU044830), Cry8Ba (e.g., Cry8Ba1, Accession #U04365), Cry8Bb (e.g., Cry8Bb1, Accession #AX543924), Cry8Bc (e.g., Cry8Bc1, Accession #AX543926), Cry8Ca (e.g., Cry8Ca1, Accession #U04366), Cry8 Da (e.g., Cry8Da1, Accession #AB089299), Cry8db (e.g., Cry8db1, Accession #AB303980), Cry8Ea (e.g., Cry8Ea1, Accession #AY329081), Cry8Fa (e.g., Cry8Fa1, Accession #AY551093), Cry8Ga (e.g., Cry8Ga1, Accession #AY590188), Cry8Ha (e.g., Cry8Ha1, Accession #EF465532), Cry8Ia (e.g., Cry8Ia1, Accession #EU381044), Cry8Ja (e.g., Cry8Ja1, Accession #EU625348), Cry8-like (Accession #ABS53003), Cry9Aa (e.g., Cry9Aa1, Accession #X58120), Cry9Ba (e.g., Cry9Ba1, Accession #X75019), Cry9Bb (e.g., Cry9Bb1, Accession #AY758316), Cry9Ca (e.g., Cry9Ca1, Accession #Z37527), Cry9 Da (e.g., Cry9Da1, Accession #D85560), Cry9db (e.g., Cry9db1, Accession #AY971349), Cry9Ea (e.g., Cry9Ea1, Accession #AB011496), Cry9Eb (e.g., Cry9Eb1, Accession #AX189653), Cry9Ec (e.g., Cry9Ec1, Accession #AF093107), Cry9Ed (e.g., Cry9Ed1, Accession #AY973867), Cry9-like (Accession #AF093107), Cry10Aa (e.g., Cry10Aa1, Accession #M12662), Cry10A-like (Accession #DQ167578), Cry11Aa (e.g., Cry11Aa1, Accession #M31737), Cry11Aa-like (Accession #DQ166531), Cry11 Ba (e.g., Cry11 Ba1, Accession #X86902), Cry11Bb (e.g., Cry11Bb1, Accession #AF017416), Cry12Aa (e.g., Cry12Aa1, Accession #L07027), Cry13Aa (e.g., Cry13Aa1, Accession #L07023), Cry14Aa (e.g., Cry14Aa1, Accession #U13955), Cry15Aa (e.g., Cry15Aa1, Accession #M76442), Cry16Aa (e.g., Cry16Aa1, Accession #X94146), Cry17Aa (e.g., Cry17Aa1, Accession #X99478), Cry18Aa (e.g., Cry18Aa1, Accession #X99049), Cry18Ba (e.g., Cry18Ba1, Accession #AF169250), Cry18Ca (e.g., Cry18Ca1, Accession #AF169251), Cry19Aa (e.g., Cry19Aa1, Accession #Y07603), Cry19Ba (e.g., Cry19Ba1, Accession #D88381), Cry20Aa (e.g., Cry20Aa1, Accession #U82518), Cry21Aa (e.g., Cry21Aa1, Accession #132932), Cry21Ba (e.g., Cry21Ba1, Accession #AB088406), Cry22Aa (e.g., Cry22Aa1, Accession #134547), Cry22Ab (e.g., Cry22Ab1, Accession #AAK50456), Cry22Ba (e.g., Cry22Ba1, Accession #AX472770), Cry23Aa (e.g., Cry23Aa1, Accession #AAF76375), Cry24Aa (e.g., Cry24Aa1, Accession #U88188), Cry24Ba (e.g., Cry24Ba1, Accession #BAD32657), Cry24Ca (e.g., Cry24Ca1, Accession #AM158318), Cry25Aa (e.g., Cry25Aa1, Accession #U88189), Cry26Aa (e.g., Cry26Aa1, Accession #AF122897), Cry27Aa (e.g., Cry27Aa1, Accession #AB023293), Cry28Aa (e.g., Cry28Aa1, Accession #AF132928), Cry29Aa (e.g., Cry29Aa1, Accession #AJ251977), Cry30Aa (e.g., Cry30Aa1, Accession #AJ251978), Cry30Ba (e.g., Cry30Ba1, Accession #BAD00052), Cry30Ca (e.g., Cry30Ca1, Accession #BAD67157), Cry30 Da (e.g., Cry30Da1, Accession #EF095955), Cry30db (e.g., Cry30db1, Accession #BAE80088), Cry30Ea (e.g., Cry30Ea1, Accession #EU503140), Cry30Fa (e.g., Cry30Fa1, Accession #EU751609), Cry30Ga (e.g., Cry30Ga1, Accession #EU882064), Cry31 Aa (e.g., Cry31Aa1, Accession #AB031065), Cry31Ab (e.g., Cry31Ab1, Accession #AB250923), Cry31Ac (e.g., Cry31Ac1, Accession #AB276125), Cry32Aa (e.g., Cry32Aa1, Accession #AY008143), Cry32Ba (e.g., Cry32Ba1, Accession #BAB78601), Cry32Ca (e.g., Cry32Ca1, Accession #BAB78602), Cry32 Da (e.g., Cry32Da1, Accession #BAB78603), Cry33Aa (e.g., Cry33Aa1, Accession #AAL26871), Cry34Aa (e.g., Cry34Aa1, Accession #AAG50341), Cry34Ab (e.g., Cry34Ab1, Accession #AAG41671), Cry34Ac (e.g., Cry34Ac1, Accession #AAG50118), Cry34Ba (e.g., Cry34Ba1, Accession #AAK64565), Cry35Aa (e.g., Cry35Aa1, Accession #AAG50342), Cry35Ab (e.g., Cry35Ab1, Accession #AAG41672), Cry35Ac (e.g., Cry35Ac1, Accession #AAG50117), Cry35Ba (e.g., Cry35Ba1, Accession #AAK64566), Cry36Aa (e.g., Cry36Aa1, Accession #AAK64558), Cry37Aa (e.g., Cry37Aa1, Accession #AAF76376), Cry38Aa (e.g., Cry38Aa1, Accession #AAK64559), Cry39Aa (e.g., Cry39Aa1, Accession #BAB72016), Cry40Aa (e.g., Cry40Aa1, Accession #BAB72018), Cry40Ba (e.g., Cry40Ba1, Accession #BAC77648), Cry40Ca (e.g., Cry40Ca1, Accession #EU381045), Cry40 Da (e.g., Cry40Da1, Accession #EU596478), Cry41Aa (e.g., Cry41Aa1, Accession #AB116649), Cry41Ab (e.g., Cry41Ab1, Accession #AB116651), Cry42Aa (e.g., Cry42Aa1, Accession #AB116652), Cry43Aa (e.g., Cry43Aa1, Accession #AB115422), Cry43Ba (e.g., Cry43Ba1, Accession #AB115422), Cry43-like (Accession #AB115422), Cry44Aa (Accession #BAD08532), Cry45Aa (Accession #BAD22577), Cry46Aa (Accession #BAC79010), Cry46Ab (Accession #BAD35170), Cry47Aa (Accession #AY950229), Cry48Aa (Accession #AJ841948), Cry48Ab (Accession #AM237207), Cry49Aa (Accession #AJ841948), Cry49Ab (e.g., Cry49Ab1, Accession #AM237202), Cry50Aa (e.g., Cry50Aa1, Accession #AB253419), Cry51Aa (e.g., Cry51Aa1, Accession #DQ836184), Cry52Aa (e.g., Cry52Aa1, Accession #EF613489), Cry53Aa (e.g., Cry53Aa1, Accession #EF633476), Cry54Aa (e.g., Cry54Aa1, Accession #EU339367), and Cry55Aa (e.g., Cry55Aa1, Accession #EU121521). In various embodiments, the insecticidal protein is a Cry1Ac or a Cry3Aa Bt delta-endotoxin.

The Bt Cry toxins that can be used in the context of present disclosure are considered to have in common that they are pore-forming proteins that cause cell lysis by producing an osmotic shock. Cry toxins share less than 40% amino acid identity with proteins from other groups. Although Cry sequences may have low similarities, their 3D structures are quite similar. In various embodiments of the present disclosure, the Cry toxin is derived from Bacillus thuringiensis strain kurstaki (Btk) HD1, which expresses Cry1Aa, Cry1Ab, Cry1Ac and Cry2Aa proteins, or from Bacillus thuringiensis strain HD73, which produces Cry1Ac (effective in controlling many leaf-feeding Lepidopterans that are important crop pests or forest pest defoliators). In various other embodiments of the present disclosure, the Cry toxin is derived from B. thuringiensis var. aizawai HD137, which produces slightly different Cry toxins such as Cry1Aa, Cry1Ba Cry1Ca and Cry1 Da (active against Lepidopteran larvae that feed on stored grains). In yet other embodiments of the present disclosure, the Cry toxin is derived from B. thuringiensis var. san diego or B. thuringiensis var. tenebrionis, which produce Cry3Aa toxin and Cry4A, Cry4B, Cry11A and Cyt1Aa toxins (active against Coleopteran pests). In still other embodiments of the present disclosure, the Cry toxin is a Cry toxin showing toxicity against mosquitoes, like Cry1, Cry2, Cry4, Cry11, and Cry29. Thus, in one embodiment of the present disclosure, the Cry toxin is derived from B. thuringiensis var. israelensis (Bti), which has been used worldwide for the control of mosquitoes.

In various embodiments of the present disclosure, the insecticidal protein is a Bt Cyt1 or Cyt2 protein. Examples of delta-endotoxins also include, but are not limited to, a DIG-3 or DIG-11 toxin, which are N-terminal deletions of alpha-helix 1 and/or alpha-helix 2 variants of Cry proteins such as Cry1A described in U.S. Pat. Nos. 8,304,604 and 8,304,605. Other Cry proteins are well known to the one of skill in the art (see, for example, Crickmore et al., “Bt toxin nomenclature” (2011), at www.lifesci.sussex.ac.uk/home/Neil_Crickmore/Bt).

The insecticidal Cry proteins produced by Bt are grouped in four different families that are not related in primary sequence, structure and probably neither in their mode of action (Zúniga-Navarrete et al. 2015, Insect Biochemistry and Molecular Biology 59, 50-57). The three-domain Cry (3d-Cry) group is the largest family of Cry proteins, with members that show toxicity against different insect orders, such as Lepidoptera, Diptera and Coleoptera. The 3d-Cry toxins are pore-forming toxins composed of three different domains. Domain I is an alpha-helix bundle that is recognized as the pore-forming domain. Domain II is a beta-prism with exposed loop regions that has been shown to be involved in recognition of larval midgut proteins, while Domain III is a beta-sandwich also involved in recognition of midgut proteins (Zúniga-Navarrete et al. 2015). Thus, domains II and III determine the specificity of Cry toxins. In various embodiments of the present disclosure, the insecticidal protein is a three-domain Cry protein or a variant or fragment thereof, wherein the variant or fragment has insecticidal activity. In other embodiments of the disclosure, the insecticidal protein is the pore-forming domain of a Cry toxin. In various embodiments of the disclosure, the insecticidal protein is the pore-forming domain of a 3d-Cry toxin. In various embodiments of the disclosure, the insecticidal protein is the domain I of a 3d-Cry toxin. In various embodiments of the disclosure, the insecticidal protein is the alpha-helix bundle of a 3d-Cry toxin. In various embodiments of the disclosure, the toxin is a modified toxin, in particular a genetically engineered Cry toxin having a deletion at the N-terminus including the domain I alpha-helix 1.

In various embodiments of the present disclosure, the insecticidal protein is a functional fragment of any insecticidal toxin or protein described herein, wherein such a functional fragment retains insecticidal activity as described herein elsewhere. In other embodiments of the present disclosure, the insecticidal protein is a functional variant of any insecticidal toxin or protein described herein, wherein such a functional variant has insecticidal activity as described herein elsewhere.

The use of Cry proteins as transgenic plant traits is well known to one skilled in the art, and Cry-transgenic plants have regularly received regulatory approval (see, e.g., Sanahuja 2011; (Plant Biotech Journal 9:283-300).

In the present disclosure, insecticidal proteins also include insecticidal lipases including, but not limited to, lipid acyl hydrolases as described in U.S. Pat. No. 7,491,869, and cholesterol oxidases such as those from Streptomyces.

Insecticidal proteins also include Vip (vegetative insecticidal proteins) toxins from Bacillus thuringiensis, e.g., such as described in U.S. Pat. Nos. 7,615,686 and 8,237,020. Vip toxins are produced during the vegetative growth phase of B. thuringiensis. At least Vip toxins Vip1/Vip2 and Vip3 have been characterized in detail and are described in the literature. Descriptions of further Vip proteins are found, for example, at www.lifesci.sussex.ac.uk/home/Neil_Crickmore/Bt/vip.html. In preferred embodiments, the insecticidal proteinis a Vip protein from B. thuringiensis. In more preferred embodiments, the insecticidal protein according to the disclosure is a Bt Vip1 or a Vip2 protein. In other preferred embodiments, the insecticidal protein is a Bt Vip3 protein. More preferably, an insecticidal protein according to the present disclosure is a Bt Vip3A protein or Bt Vip3B protein.

In various embodiments of the present disclosure, In other embodiments of the present disclosure, the insecticidal proteins include other identified or re-classified insecticidal proteins from B. thuringiensis including but not limited to Tpp, Mpp, Gpp, App, Spp, Vpa, Vpb, Mcf, Pra, Prb, Xpp, Mpf (see Table 1 of Crickmore et al. 2020, Journal of Invertebrate Pathology, 107438). One recently identified member of Vpb4 insecticidal protein family Vpb4Da2 is active against western corn rootworm (Yin et al. 2020 PLOS one, 15(11): e0242792).

In various embodiments, the insecticidal protein is a Mtx protein (mosquitocidal toxin), a Bin protein (binary toxin) or a Sip protein (secreted insecticidal toxins).

Insecticidal proteins also include toxin complex (TC) proteins, obtainable from organisms such as Xenorhabdus, Photorhabdus and Paenibacillus. As used herein, insecticidal proteins also include spider, snake and scorpion venom proteins or toxic peptides derived from these proteins.

The present disclosure also encompasses as insecticidal proteins the use of variants of Bacillus thuringiensis toxins that bind to receptors which are not natively bound by the corresponding wild-type Bacillus thuringiensis toxin. In particular, the present disclosure encompasses compositions comprising an affinity construct of the disclosure and one or more insecticidal protein(s), respectively, which comprise variants of Bacillus thuringiensis toxins that can be generated using phage-assisted continuous evolution (PACE) as described in Badran et al. 2016, Nature 533(7601): 58-63.

In various aspects, the insecticidal protein used in the context of the present invention can be fused with other proteins (or protein fragments) when forming the novel insecticidal composition.

The novel composition comprising an affinity construct of the disclosure and one or more insecticidal protein(s) also encompasses modified insecticidal proteins, e.g., insecticidal proteins that have been mutagenized, truncated, or where domains have been swapped (e.g., to enhance efficacy as described by Deist et al. 2014, Toxins, 6:3005-3027; doi:10.3390/toxins6103005). In particular, modified Bt toxins can be a useful option for maintaining Bt toxin activity in resistant insects. Truncated Cry versions may include 5′ or 3′ truncations, leading to deletions of N- and C-terminal Cry protein domains. Modified toxins that can be used in the context of the affinity constructs in the present disclosure may also include toxins with an altered GC content in their DNA sequence, such as a GC content that mimics eukaryotic genes. These modifications may, e.g., enhance their expression in eukaryotic systems used for the production of crystal Bt protein that can be used in topical application systems in plants according to the present disclosure. These modifications may also enhance the expression of the insecticidal proteins in the context of the present disclosure of transgenic plants or microorganism, or may enhance the co-expression of the affinity construct of the present disclosure and an insecticidal protein in transgenic plants or microorganism in a method for protecting a plant against an insect pest according to the present disclosure.

Modified insect toxins that can be used in the context of the present invention, for example, to form the novel insecticidal compositions comprising an affinity construct of the disclosure and one or more insecticidal protein(s) may also include changes in protease cleavage sites, such as proteins with altered sequences obtained by site-directed mutagenesis or by using genome-editing tools. Furthermore, said modified insecticidal toxins may include proteins that are already chimeric proteins or fusion proteins, consisting of a toxin and another protein or peptide that enables or increases binding of the toxin to the insect target tissue/membrane. Such fusion proteins may include lectins, or specific gut-binding peptides.

In some embodiments the insecticidal proteins, which form part of a novel composition of the disclosure comprising an affinity construct and one or more insecticidal protein(s) have amino acid sequences that are shorter than the full-length sequences, either due to the use of an alternate downstream start site or due to processing that produces a shorter protein having insecticidal activity. Such processing may occur in the target organism after the insecticidal protein is ingested by the pest.

Thus, provided herein are novel isolated or recombinant nucleic acid sequences encoding the novel affinity constructs of the present disclosure. Also provided are the amino acid sequences of the novel affinity constructs of the disclosure. The protein resulting from translation of the genes encoding for the novel affinity constructs in combination with the respective insecticidal protein the at least one affinity molecule B is directed against allows controlling or killing pests that ingest same.

In various embodiments, the affinity construct is soluble in the gut of an insect or an insect larva.

### Construction of the Affinity Constructs of the Disclosure

In the context of the affinity constructs comprising at least one affinity molecule A and at least one affinity molecule B, any affinity mediating molecule as defined above (for example, selected from the group comprising a protein, carbohydrate, lipid or nucleotide, or a fragment, derivative or variant of any of these), including monoclonal antibodies as widely applied in medicine and in molecular biology research, may be used (reviewed in Nature Reviews Immunology 10, 285 (2010), FIG. 1). Preferably, the affinity molecule(s) (i.e., the at least one affinity molecule A and/or the at least one affinity molecule B) is/are a protein which is a non-antibody binding protein or an antibody or a fragment, derivative or variant thereof. More preferred, the non-antibody binding protein is any one of affimers (adhirons), affibodies, affilins, affitins, nanofitin, alphabodies (triple helix coiled coil), anticalins, lipocalins, avimers, DARPins (ankyrin repeat), fynomer, kunitz domain pepties, monobodies, adnectins, trinectins, nanoCLAMPs, cellulose/carbohydrate binding molecule (CBM) (for example, dockerins or lectins), centyrins, pronectins, and fibronectin or a fragment, derivative or variant of any of these. In other embodiments, the antibody is a naturally occurring antibody or a fragment, derivative or variant thereof, in particular a nanobody or an immunoglobulin gamma (IgG). Preferably, the fragment of the naturally occurring antibody can be an antibody fragment selected from the group comprising a Fab fragment, a single heavy chain and a single light chain, a single chain variable fragment, a V_{H}H fragment, CDR3 region and a bispecific monoclonal antibody (diabody). The Fab fragment can occur as monomer or as a linked dimer, or antibody fragments consisting of a single heavy chain and a single light chain, or consisting of the heavy chain with all three domains (so called V_{H}H), two domains or only on domain of the constant region (the so called crystallizable Fragment Fc) or the single light chain or the region facilitating the recognition to the antigen comprising the CDR3 region as will be described in more detail further below. Encompassed are also synthetic affinity molecules like three helix coils. In other preferred embodiments, the nucleotide is a RNA aptamer, a SOMAmer or a ribozyme or a fragment, derivative or variant thereof.

In the context of the affinity construct comprising at least one affinity molecule A and at least one affinity molecule B, the affinity molecules (i.e., the at least one affinity molecule A and/or the at least one affinity molecule B) can be designed in a way to bind more than one target, e.g., two, three, four or even more targets, thus being bispecific, trispecific, tetraspecific or multispecific. With regards to the construction of a multispecific affinity molecule the affinity molecules can be fused directly or by using a linker, which does not interfere with the structure and function of the proteins, or fragments thereof, to be linked.

Affinity construct comprising at least one affinity molecule A and at least one affinity molecule B having the structure (Am-Ln-Bo)p

In various embodiments, the novel affinity construct provided by the present disclosure and as described above comprising at least one affinity molecule A, and at least one affinity molecule B, which are optionally separated by a linker L comprising at least one amino acid, may have the structure (Am-Ln-Bo)p. In such embodiments, A is the affinity molecule A or a fragment thereof as described above, B is the affinity molecule B or a fragment thereof as described above, the integer m is at least 1, the integer o is also at least 1, L is a linker comprising or consisting of at least one amino acid, the integer n can be 0 or larger, and the integer p is at least 1. Integers m, n and o can have different values. Embodiments describing specific values of the integers m, n and o are described herein below. Furthermore, the affinity molecule A, the linker L and the affinity molecule B are all covalently bound to form the affinity construct of the structure Am-Ln-Bo. Thus, the present disclosure encompasses a novel affinity construct of the structure Am-Ln-Bo comprising at least one affinity molecule A, at least one affinity molecule B, and optionally at least one linker L, wherein the affinity molecule A or a fragment thereof is capable of recognizing an insect-specific structure in and/or on a target insect, and affinity molecule B or a fragment thereof is capable of binding an insecticidal protein (toxin), and wherein the integer m is at least 1 and the integer o is also at least 1, wherein L is a linker comprising or consisting of at least one amino acid, and wherein the integer n can be 0 or larger, and wherein the integers m, n and o can have different values, and wherein A, L and B are all covalently bound to form said affinity construct. If the integer n is 0 (zero), the affinity molecule A and the affinity molecule B are covalently bound to form the affinity construct of the structure Am-Ln-Bo or Am-Bo, respectively. Several units of the affinity construct Am-Ln-Bo or Am-Bo and be fused together to form affinity molecules of higher order. The integer p indicates how many affinity constructs Am-Ln-Bo or Am-Bo are fused together. For the structure Am-Ln-Bo-Am-Ln-Bo the integer p would be 2, for the structure Am-Ln-Bo-Am-Ln-Bo-Am-Ln-Bo it would be 3 and so on.

The affinity construct of the structure Am-Ln-Bo can be expressed in a transgenic plant or microorganism or be applied as an insecticidal spray/solution to a plant, seed or insect, when applied along with along with an insecticidal protein (toxin), wherein the insecticidal protein (toxin) corresponds to the insecticidal protein (toxin) which the at least one affinity molecule B is (capable of) binding to, or directed to.

In various embodiments of the affinity construct of the disclosure having the structure Aₘ-Lₙ-Bₒ, the integer m may be any one of 1, 2, 3, 4, 5, 6, 7, 8, 9, 10 or more. Preferably, the integer m is any one of 1, 2 and 3, more preferably 1 or 2, and even more preferably the integer m is 1. Furthermore, in various embodiments of the affinity construct of the disclosure having the structure Am-Ln-Bo, the integer o may be any one of 1, 2, 3, 4, 5, 6, 7, 8, 9, 10 or more. Preferably, the integer o is any one of 1, 2 and 3, more preferably 1 or 2, and even more preferably the integer o is 1. Still further, in various embodiments of the affinity construct of the disclosure having the structure Aₘ-Lₙ-Bₒ, the integer n may be any one of 0, 1, 2, 3, 4, 5, 6, 7 8, 9, 10 or more. Preferably, the integer n is any one of 1, 2 and 3, more preferably 1 or 2, and even more preferably the integer n is 1.

In various embodiments, the affinity construct of the disclosure having the structure A1L1-Bo comprises at least one affinity molecule A or a fragment thereof, and at least one affinity molecule B or a fragment thereof, and one linker L comprising or consisting of at least one amino acid, wherein the integer o (and thus the number of affinity molecules B in the affinity construct) is any one of 1, 2 or 3, preferably the integer o is 1 or 2.

In preferred embodiments, the affinity construct of the disclosure having the structure Am-L1-Bo comprises at least one affinity molecule A, at least one affinity molecule B, and one linker L comprising or consisting of at least one amino acid, and wherein the integer m is at least 1 and the integer o is at least 1.

In the present disclosure, the terms “insecticidal protein”, “insecticidal toxin”, and “insecticidal protein toxin”” may be used interchangeably.

[1] Affinity construct comprising (1) at least one affinity molecule A capable of recognizing, or capable of binding to, or binding to, or being directed to, or being designed to bind to an insect-specific structure in and/or on a target insect, and (2) at least one affinity molecule B capable of binding to, or binding to, or being directed to, or being designed to bind to an insecticidal protein (toxin), wherein the at least one affinity molecule A and the at least one affinity molecule B are optionally separated by a linker L comprising at least one amino acid.

[2] The affinity construct according to [1], wherein the at least one affinity molecule A is different from the at least one affinity molecule B.

[3] The affinity construct according to [1] or [2], wherein the at least one affinity molecule A has one or more binding sites (valences) for the same or different insect-specific structures in and/or on a target insect and wherein the at least one affinity molecule B has one or more binding sites (valences) for the same or different insecticidal protein (toxins).

[4] The affinity construct according to any one of [1] to [3], wherein the at least one affinity molecule A specifically binds to a receptor, more specifically a membrane-bound receptor, of an inner organ of the target insect.

[5] The affinity construct according to any one of [1] to [4], wherein the at least one affinity molecule A specifically binds to a receptor, more specifically a membrane-bound receptor, of the digestive tract, of a reproductive organ or of the nervous system.

[6] The affinity construct according to any one of [1] to [5], wherein the insecticidal protein (toxin) is selected from the group consisting of crystal toxins (Cry and Cyt proteins), vegetative insecticidal toxins (Vip proteins), mosquitocidal toxins (Mtx proteins), binary toxins (Bin proteins), and secreted insecticidal toxins (Sip proteins), as well as fragments or multimers thereof.

[7] The affinity construct according to any one of [1] to [6], wherein the insecticidal protein is derived from Bacillus thuringiensis.

[8] The affinity construct according to any one of [1] to [7], wherein the at least one affinity molecule A and the at least one affinity molecule B are an affinity mediating molecule selected from the group comprising a protein, carbohydrate, lipid or nucleotide, or a fragment, derivative or variant of any of these, wherein the at least one affinity molecule A and the at least one affinity molecule B are identical or different.

[9] The affinity construct according to [8], wherein the protein is a non-antibody binding protein or an antibody or a fragment, derivative or variant thereof.

[10] The affinity construct according to [9], wherein the non-antibody binding protein is selected from the group comprising affimers (adhirons), affibodies, affilins, affitins, nanofitin, alphabodies (triple helix coiled coil), anticalins, lipocalins, avimers, DARPins (ankyrin repeat), fynomer, kunitz domain pepties, monobodies, adnectins, trinectins, nanoCLAMPs, cellulose/carbohydrate binding molecule (CBM) (for example, dockerins or lectins), centyrins, pronectins, and fibronectin or a fragment, derivative or variant of any of these.

[11] The affinity construct according to [9], wherein the antibody is naturally-occurring antibody or a fragment, derivative or variant thereof.

[12] The affinity construct according to claim [11], wherein the naturally-occurring antibody or a fragment, derivative or variant thereof is a nanobody or an immunoglobulin gamma (IgG).

[13] The affinity construct according to [12], wherein the fragment of the naturally-occurring antibody is an antibody fragment selected from the group comprising a Fab fragment, a single heavy chain and a single light chain, a single chain variable fragment, a V_{H}H fragment, CDR3 region and a bispecific monoclonal antibody (diabody).

[14] The affinity construct according to [8], wherein the nucleotide is a RNA aptamer, a SOMAmer or a ribozyme or a fragment, derivative or variant thereof.

[15] An insecticidal composition comprising the affinity construct according to any one of [1] to [5] and at least one insecticidal protein (toxin), wherein the at least one insecticidal protein (toxin) corresponds to the insecticidal protein(s) (toxin(s)), which the at least one affinity molecule B is capable of binding to, or is binding to, or is being directed to, or is being designed to bind to.

[16] Use of the affinity construct according to any one of [1] to [14], or of the insecticidal composition of [15] for protecting a plant, plant part or plant seed against one or more insect pest(s).

[17] A method of protecting a plant or plant parts or plant seeds against one or more insect pest(s) comprising

(a) co-expressing the affinity construct according to any one of [1] to [14] together with one or more insecticidal protein(s) (toxin(s)) in a plant, plant parts or plant seeds, wherein the one or more insecticidal protein(s) (toxin(s)) correspond(s) to the insecticidal protein(s) (toxin(s)) which the at least one affinity molecule B is capable of binding to, or is binding to, or is being directed to, or is being designed to bind to, or

(b) (co-)expressing the affinity construct according to any one of [1] to [14] and one or more insecticidal protein(s) (toxin(s)) in one or more microorganism(s) followed by the application of the one or more microorganism(s) (co-)expressing the affinity construct and the one or more insecticidal protein(s) (toxin(s)) either in purified form or together with the respective culture medium/media to a plant, plant parts or plant seeds, wherein the one or more insecticidal protein(s) (toxin(s)) correspond(s) to the insecticidal protein(s) (toxin(s)) which the at least one affinity molecule B is capable of binding to, or is binding to, or is being directed to, or is being designed to bind to; or

(c) expressing the affinity construct according to any one of [1] to [14] in a plant, plant parts or plant seeds and applying the one or more insecticidal protein(s) (toxin(s)) to the plant, plant parts or plant seeds the at least one affinity molecule B comprised in the affinity construct is capable of binding to, or is binding to, or is being directed to, or is being designed to bind to, wherein said one or more insecticidal protein(s) (toxin(s)) are applied in purified form or by applying the microorganism(s) expressing these insecticidal protein(s) (toxin(s)); or

(d) expressing the one or more insecticidal protein(s) (toxin(s)) in a plant, plant part or plant seed the at least one affinity molecule B comprised in the affinity construct is capable of binding to, or is binding to, or is being directed to, or is being designed to bind to, and applying the affinity construct according to any one of [1] to [14] to the plant, plant parts or plant seeds, wherein said affinity construct is expressed in one or more microorganism and is applied to said plant, plant parts or plant seeds either in purified form or by applying the microorganism(s) expressing the affinity construct; or

(e) applying to the plant or plants parts or plant seeds the insecticidal composition of [15].

[18] A method of producing a plant or a microorganism comprising the affinity construct according to any one of [1] to [14] and one or more insecticidal protein(s) (toxin(s)), the method comprising co-expressing in a plant or microorganism the affinity construct according to any one of [1] to [14] and one or more insecticidal protein(s) (toxin(s)), wherein said one or more insecticidal protein(s) (toxin(s)) correspond(s) to the insecticidal protein(s) (toxin(s)) which the at least one affinity molecule B is capable of binding to, or is binding to, or is being directed to, or is being designed to bind to.

[19] The method of [17(a)] or [18], comprising the step of transforming the plant or microorganism with one or more nucleic acid molecules encoding the affinity construct according to any one of [1] to [14], and one or more nucleic acid molecules encoding the insecticidal protein(s) (toxin(s)), wherein said one or more insecticidal protein(s) (toxin(s)) correspond(s) to the insecticidal protein(s) (toxin(s)) which the at least one affinity molecule B is capable of binding to, or is binding to, or is being directed to, or is being designed to bind to.

[20] A method of producing an insecticidal formulation comprising the affinity construct according to any one of [1] to [14] and one or more insecticidal protein(s) (toxin(s)), the method comprising formulating the affinity construct according to any one of [1] to [14] and one or more insecticidal protein(s) (toxin(s)) as insecticidal formulation, wherein said one or more insecticidal protein(s) (toxin(s)) correspond(s) to the insecticidal protein(s) (toxin(s)) which the at least one affinity molecule B is capable of binding to, or is binding to, or is being directed to, or is being designed to bind to, and wherein said affinity construct and said one or more insecticidal protein(s) (toxin(s)) are expressed in one or more microorganism,

[21] The method of [20], wherein the affinity construct and the one or more insecticidal protein(s) (toxin(s)) being expressed in one or more microorganism are added to the insecticidal composition in either purified form or by adding the microorganism(s) expressing the affinity construct and the one or more insecticidal protein(s).

[22] A plant, plant part or plant seed or a microorganism comprising
(i) one or more nucleic acid molecules encoding the affinity molecule according to any one of [1] to [14], and/or one or more nucleic acid molecules encoding one or more insecticidal protein(s) (toxin(s)), wherein said one or more insecticidal protein(s) (toxin(s)) correspond(s) to the insecticidal protein(s) (toxin(s)) which the at least one affinity molecule B is capable of binding to, or is binding to, or is being directed to, or is being designed to bind to, or
(ii) one or more vectors comprising the one or more nucleic acid molecules of (i).

[23] A plant, plant part or plant seed, a microorganism or insecticidal formulation, produced or obtainable by the method according to any one of [17] to [21].

In the present disclosure, the affinity molecule or a fragment thereof as described above can be fused to at least a second affinity molecule or fragment thereof. In the present disclosure, an affinity molecule A or a fragment thereof as described above and an affinity molecule B or a fragment thereof as described above can be fused by using a linker, which does not interfere with the structure and function of the two proteins fused or any fragments thereof.

In various embodiments of the disclosure, the novel affinity construct may comprise more than one affinity molecule A or fragments thereof and/or more than one affinity molecule B or fragments thereof. The said more than one insecticidal protein or fragments thereof may be linked by chemical cross-linking.

The affinity constructs of the present disclosure can bind—via one or more affinity molecule(s) B—to insect-specific structures (receptors) that are otherwise not naturally bound, i.e., that are otherwise not target structures (receptors) of the insecticidal protein. The same applies with respect to the insecticidal protein, which is part of the composition of the disclosure comprising a novel affinity construct of the disclosure and an insecticidal protein. This binding to the insect-specific structures (receptors) serves to enrich the insecticidal protein to the gut membrane of the insect, and thereby aids membrane integration and pore formation of the insecticidal protein. It is to be understood that an affinity molecule of the present disclosure may comprise affinities to more than insecticidal protein (toxin). Further, it is also to be understood that a composition of the disclosure comprising a novel affinity construct of the disclosure and an insecticidal protein, may comprise more than one insecticidal protein (toxin). In addition to that it is also to be understood that a transgenic plant according to the present disclosure to which the novel affinity construct of the disclosure is applied may be expressing more than one insecticidal protein (toxin). For example, these more than one insecticidal protein can be (1) several units or copies of the same insecticidal protein, or (2) one or more copies of a particular first insecticidal protein in combination with one or more copies of a particular second insecticidal protein. In case of the latter, it is also considered that the “more than one insecticidal protein” can be one or more copies of a particular first insecticidal protein in combination with one or more copies of a particular third, fourth, fifth etc. insecticidal protein.

### Plant Applications

The present disclosure encompasses the use of an affinity construct as described above comprising at least one affinity molecule A and at least one affinity molecule A together with at least one insecticidal protein (toxin), wherein the insecticidal protein (toxin) corresponds to the insecticidal protein (toxin) which the at least one affinity molecule B is capable of binding to, or binding to, or being directed to, or being designed to bind as described above, for protecting a plant against an insect pest.

The use may encompass in general the introduction of the novel affinity construct of the present disclosure comprising at least one affinity molecule A and at least one affinity molecule B into a plant, plant cell or plant seed on one hand or into microorganism on the other hand by means known to the person skilled in the art. The present disclosure also encompasses in general the introduction of the insecticidal protein (toxin), which the at least one affinity molecule B of the novel affinity construct of the present disclosure is capable of binding to, or is binding to, or is being directed to, or being designed to bind to, into a plant, plant cell or plant seed on one hand or into microorganism on the other hand by means known to the person skilled in the art.

In particular, the present disclosure encompasses a method for protecting a plant against an insect pest comprising co-expressing in a plant, plant part or plant seed the affinity construct of the present disclosure comprising at least one affinity molecule A and at least one affinity molecule B together with one or more insecticidal protein (toxin), wherein the one or more insecticidal protein (toxin) correspond(s) to the insecticidal protein (toxin) which the at least one affinity molecule B is capable of binding to, or is binding to, or is being directed to, or is being designed to bind to. In this context, both the affinity construct of the present invention and the one or more insecticidal protein (toxin), which the at least one affinity molecule B is capable of binding to, or is binding to, or is being directed to, or being designed to bind to, are introduced into the plant, plant part or plant seed by means known to the person skilled in the art. Upon expression in the plant, an insect would take up the affinity constructs well as the insecticidal protein(s). The affinity construct is then directed and bound to an insect-specific structure in or on the insect pest via the corresponding at least one affinity molecule A in the affinity construct, which is capable of recognizing, or is capable of binding to, or is binding to, or is being directed to, or is being designed to bind to an insect-specific structure in and/or on a target insect. The insecticidal activity of the affinity construct is enhanced through the higher binding affinity of the multi-specific affinity molecule to insect-specific structures, preferably to insect receptors.

In a further preferred embodiment of the method for protecting a plant against an insect pest, the affinity construct of the present invention and one or more insecticidal protein(s) (toxin(s)) are co-expressed in one or more microorganism(s) followed by the application of the one or more microorganism(s) co-expressing the affinity construct and the one or more insecticidal protein(s) (toxin(s)) either in purified form or together with the respective culture medium/media to a plant, plant parts or plant seeds. In these embodiments the one or more insecticidal protein(s) (toxin(s)) correspond(s) to the insecticidal protein(s) (toxin(s)) which the at least one affinity molecule B is capable of binding to, or is binding to, or is being directed to, or is being designed to bind to. In this context both the affinity construct of the present invention and the insecticidal protein (toxin), which the at least one affinity molecule B is capable of binding to, or is binding to, or is being directed to, or being designed to bind to, introduced into one or more microorganism(s) by means known to the person skilled in the art. Upon feeding on the plant, plant part or plant seed, an insect would take up the affinity construct applied to the plant material as well as the insecticidal protein(s) expressed in the plant material. The affinity construct is then directed and bound to an insect-specific structure in or on the insect pest via the corresponding at least one affinity molecule A in the affinity construct, which is capable of recognizing, or is capable of binding to, or is binding to, or is being directed to, or is being designed to bind to an insect-specific structure in and/or on a target insect. The insecticidal activity of the affinity construct is enhanced through the higher binding affinity of the multi-specific affinity molecule to insect-specific structures, preferably to insect receptors.

The use also encompasses the introduction of the affinity construct of the present invention into a plant, plant part or plant seed by means known to the person skilled in the art. Such use may encompass applying to a plant, plant part or plant seed that is transformed with the affinity construct of the present invention a formulation comprising the insecticidal protein (toxin), which corresponds to the insecticidal protein (toxin) which the at least one affinity molecule B is capable of binding to, or is binding to, or is being directed to, or being designed to bind, preferably by way of a spray. The spray formulation applied to the plant comprises the insecticidal protein(s) for which the at least one affinity molecule B is capable of binding, or binding to, or being directed to, or being designed to bind to. In another embodiment, the use also encompasses the introduction of the insecticidal protein (toxin), which corresponds to the insecticidal protein (toxin) which the at least one affinity molecule B is capable of binding to, into a plant, plant part or plant seed by means which are known to the person skilled in the art. This particular use further encompasses the introduction of the novel affinity construct of the present disclosure into one or more microorganism(s) by means known to the person skilled in the art and applying the affinity construct to said plant, plant parts or plant seeds either in purified form or by applying the microorganism(s) expressing the affinity construct. Such use may encompass microorganism transformed with the novel affinity construct of the present invention formulated as a composition, preferably formulated as a spray.

In more preferred embodiments, the use encompasses the application of the insecticidal composition or the spray of the present invention comprising the affinity construct of the present disclosure and the insecticidal protein (toxin), which the at least one affinity molecule B is capable of binding to, or is binding to, or is being directed to, or being designed to bind to, to the surface of a plant. Alternatively, the affinity construct of the present disclosure and/or the insecticidal protein (toxin), which the at least one affinity molecule B is capable of binding to, or is binding to, or is being directed to, or being designed to bind to, may be extracted from the microorganism transformed with said affinity construct of the present disclosure and/or said insecticidal protein (toxin) and then formulated as a composition, preferably formulated as a spray. In preferred embodiments, said use encompasses the application of the composition or the spray comprising the affinity construct of the present invention and/or the insecticidal protein (toxin), which the at least one affinity molecule B is capable of binding to, or is binding to, or is being directed to, or being designed to bind to, extracted from the microorganisms to the surface of a plant.

Upon feeding on the plant, an insect would take up the affinity construct of the present disclosure as well as the insecticidal protein(s). The insecticidal protein is then directed and bound to a receptor target in the insect via the corresponding affinity molecule(s) A in the affinity construct, which is capable of recognizing, or capable of binding to, or binding to, or being directed to, or being designed to bind to a receptor in and/or on a target insect. The insecticidal activity of the insecticidal protein is enhanced through the higher binding affinity of the multispecific affinity molecule to insect receptors.

In any of the above embodiments, the plant or the microorganism may preferably be modified by using known genome editing tools for delivery of constructs, through either Agrobacterium-mediated transfer, electroporation, micro-projectile bombardment, virus-mediated delivery or sexual cross. The techniques of Agrobacterium-mediated transfer, electroporation, micro-projectile bombardment, virus-mediated delivery and sexual cross are well known to the skilled person and corresponding methods are described in the literature. The same holds for genome editing tools like, e.g., TAL Effector Nucleases (TALEN), CRISPR/Cas9 etc.

### Genetically Modified (GM) or Gene Edited (GE) Plants

The present disclosure encompasses co-expression of one or more insecticidal protein (toxins) that have been genetically modified (GM) or gene edited (GE) with one or more affinity constructs of the present invention. Preferably, the affinity constructs comprise affinity mediating molecules in crop plants. The skilled artisan will further appreciate that changes can be introduced into the nucleic acid sequences coding for insecticidal proteins by GM or GE approaches thereby leading to changes in the amino acid sequence of the encoded the insecticidal protein (toxin) used in the context of the present invention without altering the biological activity of the proteins. Thus, variant nucleic acid molecules can be created by introducing one or more nucleotide substitutions, additions or deletions into the corresponding nucleic acid sequence disclosed herein, such that one or more amino acid substitutions, additions or deletions are introduced into the encoded protein. Mutations can be introduced by standard techniques, such as site-directed mutagenesis and PCR-mediated mutagenesis. Mutations may also be introduced using genome editing tools like, e.g., Zinc Finger Nucleases, TAL Effector Nucleases (TALEN), and CRISPR/Cas systems, like, for example, CRISPR/Cas9 and CRISPR/cpf1. Specifically, the present disclosure encompasses co-expression of one or more insecticidal proteins (toxins) as disclosed herein, in particular GM/GE insecticidal proteins (toxins) as disclosed herein, with one or more affinity constructs comprising at least one affinity molecule A and at least one affinity molecule B of the present disclosure in crop plants. Example of such GM/GE insecticidal protein (toxin) could be any Bt protein evolved/mutated to have increased affinity to its existing or to novel membrane bound receptor proteins. Example of such insecticidal protein (toxins) that have been gene edited (GE) could be any native protease inhibitor protein evolved/mutated to have increased affinity to its existing or to novel protease receptor proteins.

### GM/GE Microbial Sprays & GM/GE Microbial Seed Treatments

The present disclosure encompasses co-expression of one or more insecticidal protein (toxins) that have been genetically modified (GM) or gene edited (GE) as mentioned above, and one or more affinity construct of the present disclosure in currently existing and commercially used Bacillus thuringiensis strains expressing Bt toxins.

The present disclosure further encompasses co-expression of one or more insecticidal protein (toxins) that have been genetically modified (GM) or gene edited (GE) as mentioned above, and one or more affinity construct of the present disclosure in other microbes (e.g., Lactobacillus, Agrobacterium, plant endophytic microbes such as Azotic's Gluconacetobacter diazotrophicus, any other microbes pursued by Biologics companies such as Indigo, AgBiome and the like).

Non-GM Bacillus thuringiensis-based Sprays & Seed Treatments

The present disclosure encompasses co-formulation of one or more Bacillus thuringiensis strains each expressing specific (combinations) of Bt toxins with one or more affinity construct of the present disclosure (comprising at least one affinity molecule A and at least one affinity molecule B) in spray or seed formulations. The affinity constructs of the present disclosure are considered to confer increased affinity binding of the respective Bt toxins to their existing or novel membrane bound receptor proteins.

The present disclosure further encompasses co-formulation of one or more purified (and stabilized) Bt toxins or any other type of toxins with one or more affinity construct of the present disclosure (comprising at least one affinity molecule A and at least one affinity molecule B)) in spray or seed formulations. The affinity constructs of the present disclosure are considered to confer increased affinity binding of the respective purified/stabilized Bt toxins and other type of toxins to their existing or novel membrane bound receptor proteins, or existing or novel toxin receptor proteins, respectively. Example of other type of toxins could again be different types of protease inhibitors and their respective protease receptor proteins.

### Linker Molecules

The affinity molecules (or fragments thereof) comprised in the affinity constructs of the present invention can be fused directly or by using a (flexible) linker which does not interfere with the structure and function of the proteins (or fragments thereof) to be linked. In various embodiments, the linker (linker L) is a flexible linker. Such flexible linkers may be, for instance, those which are used to fuse the variable domains of the heavy and light chain of conventional immunoglobulins to construct a single chain antibody, scFv, or may be those used to create bivalent bispecific scFvs, or may be those used in immunotoxins. In preferred embodiments, the linker is an amino acid linker, more preferably a flexible amino acid linker. The terms “amino acid(s)” and “amino acid residue(s)” may be used herein interchangeably. In various embodiments, the amino acid linker is a peptide linker, more preferably a flexible peptide linker. Linkers to be used in the present disclosure may also be based on hinge regions found in antibody molecules (Pack et al. 1993, Biotechnology (NY) 11, 1271-1277; Pack and Plückthun, 1992, Biochemistry 31, 1579-1584), or may be based on peptide fragments between structural domains of proteins.

A linker can be used for fusing one affinity molecule or a fragment thereof to another affinity molecule or a fragment thereof to form the affinity construct of the present invention. For example, one affinity molecule A is fused to one affinity molecule B either directly or using linker as described herein.

The term “directly” defines fusions in which the single affinity molecule or a fragment thereof is joined without a linker. As explained herein above, in preferred embodiments, the linker L is an amino acid linker, or a peptide or polypeptide linker. The linking group may be a polypeptide of between 1 and 500 amino acids in length. Preferably, the linking group or linker comprises between 1 and 100 amino acids in length, more preferably between 1 and 50 amino acids in length, still more preferably between 1 and 40, between 1 and 30, between 1 and 20, or between 1 and 10 amino acids in length.

A linker according to the present disclosure may be a flexible linker, which does not interfere with the structure and function of the affinity molecules to be linked. This applies to both the at least two affinity molecules to be fused/linked/joined for generating the novel affinity constructs of the disclosure. Said flexible linkers are, for instance, those which have been used to fuse the variable domains of the heavy and light chain of immunoglobulins to construct a scFv, those used to create bivalent bispecific scFvs or those used in immunotoxins (see, for example, Huston et al. 1992; Takkinen et al. 1991). Linkers can also be based on hinge regions in antibody molecules (Pack and Plückthun, 1992; Pack et al. 1993) or on peptide fragments between structural domains of proteins. Fusions can be made between the multivalent affinity molecules at both sides, the C- and N-terminus.

Further, the linker according to the present disclosure may be a linker which does interfere with the structure and function of one or more affinity molecules to be linked in a positive manner; e.g. by activating the one and/or the other affinity molecule being comprised in the affinity construct in instances where the affinity molecule are not active without the interference of the linker. This activation may, for example, be the results of a change in the 3-dimensional structure that affects the binding efficiency in a positive way.

A linker can be designed as a flexible GGGS-linker of, for example, three distinct lengths (9, 25, 35 amino acids containing glycine for flexibility and serine for solubility), as fusion head-to-tail with a 9 amino acid glycine/serine linker (preferred option) or as hinge-sequence added to the 3′ extremity of an affinity molecule.

The linkers joining the at least two affinity molecules of the novel affinity construct of the present disclosure (i.e., the at least one affinity molecule A and the at least one affinity molecule B as described above) are preferably designed to (1) allow the at least two molecules to fold and act independently of each other, (2) not have a propensity for developing an ordered secondary structure which could interfere with the functional domains of the two proteins, (3) have minimal hydrophobic or uncharged characteristic which could interact with the functional protein domains and (4) provide steric separation of the two molecules such that they can interact simultaneously with their corresponding targets or receptors on a single cell or on multiple cells within the target tissue (e.g., the midgut). Typically surface amino acids in flexible protein regions include Gly, Asn and Ser. Virtually any permutation of amino acid sequences containing Gly, Asn and Ser would be expected to satisfy the above criteria for a linker sequence. Other neutral amino acids, such as Thr and Ala, may also be used in the linker sequence. Additional amino acids may also be included in the linkers due to the addition of unique restriction sites in the linker sequence to facilitate construction of the fusions.

In various embodiments the linkers may comprise sequences selected from the group of formulas: (Gly3Ser)n, (Gly4Ser)n, (Gly5Ser)n, (GlynSer)n and (AlaGlySer)n, where n is an integer which can be 1 or more, preferably any one of 1, 2, 3, 4, 5, 6, 7, 8, 9, and 10. The length of the amino acid sequence of the linker can be selected empirically or with guidance from structural information or by using a combination of the two approaches. In various preferred embodiments, the linker comprises a sequence of the formula (Gly4Ser)n, where n is an integer which can be 1 or more, including any one of 1, 2, 3, 4, 5, 6, 7, 8, 9, and 10. Preferably, the integer is 1. Accordingly, in various preferred embodiments, the linker has a sequence GGGGSGGGG (SEQ. ID NOS. 36).

Those skilled in the art will recognize that there are many such sequences that vary in length or composition that can serve as linkers with the primary consideration being that they be neither excessively long nor short. Sequences of affinity structures or affinity molecules of the disclosure capable of folding to biologically active states can be prepared by appropriate selection of the beginning (amino terminus) and ending (carboxyl terminus) positions from within the original polypeptide chain while using the linker sequence as described above.

### Valences

The valence is the number of binding sites of a single affinity molecule and therefore the capability of said molecule to recognize a certain target (via its so-called paratope, whereas the region on the target is called the epitope). The presence of more than one valence can improve avidity, which is defined as accumulated strength of multiple binding sites and can exceed the mere sum of its individual binding sites. The human IgG is bi-valent; it consists of an antibody molecule with two binding-sites each for its epitope. The human IgM is dekavalent (deka stands for gr. “ten”) because it consists of five bivalent antibody molecules with two binding sites each generating 10 binding sites in total.

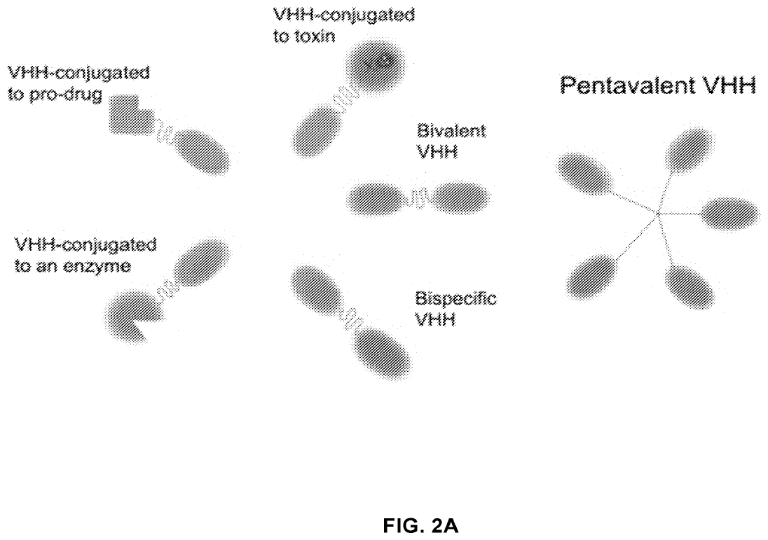

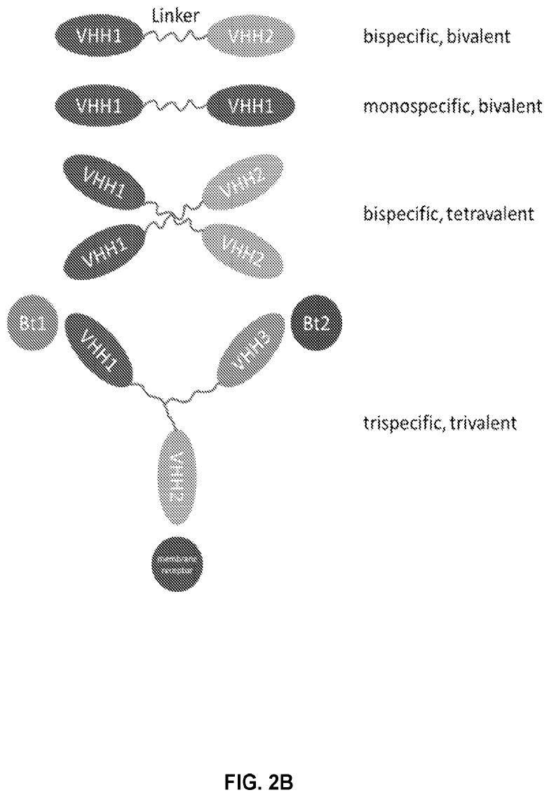

Valences might be of higher order to improve potency and affect avidity, see FIGS. 1 and 2A and 2B. Examples are divalent, trivalent, tetravalent, pentavalent, or multivalent, i.e. having two, three, four, five or many binding sites, respectively (see FIG. 1).

Affinity molecules of the present disclosure, i.e. the one or more affinity molecule A and the one or more affinity molecule B of the present invention, can be designed in a way to bind more than one target, e.g. two, three, four or even more targets, thus being bispecific, trispecific, tetraspecific or multispecific. As an example it should be noted, that an affinity construct of the present invention can have a multitude of affinity molecules; for example two affinity molecules, which are directed at a single epitope of the insect-specific structure, for example an receptor, in or on the insect pest, so that this affinity molecule has one specificity but two valences for this epitope, and additionally contains at least one affinity molecule B which is directed against an epitope of the insecticidal protein. The entire molecule would then be di-specific, i.e. detecting two distinct epitopes, and trivalent, since the affinity molecule has three binding sites in total. Options that may be employed to the affinity molecules of the present disclosure:

Binding
C-terminal to linker: may reduce affinity (Conrath et al. 2001).
N-terminal to linker.

Linker
Flexible GGGS-linker of three distinct lengths (9, 25, 35 amino acids containing glycine for flexibility and serine for solubility).
Fusion head-to-tail with a 9 amino acid glycine/serine linker.
Hinge-sequence added to 3′ extremity of V_{H}H as linker.

Valences: higher valences may improve potency and affect avidity, see FIGS. 1 and 2.
divalent
Trivalent
Tetravalent
Multivalent

Specificity
Bispecific
Trispecific
Tetraspecific

### Application of Multispecific Affinity Molecules

One of the main aspects of the present disclosure is to apply the purified multispecific affinity molecules of the present invention (i.e., the affinity construct comprising at least one affinity molecule A, and at least one affinity molecule B) to the plant (e.g., by spraying) together with the insecticidal protein(s) for which affinity was generated (via affinity molecule B). Upon feeding on the plant, an insect would then take up the affinity molecule(s) (i.e., the affinity construct comprising at least one affinity molecule A and at least one affinity molecule B) as well as the insecticidal protein(s)). The oligomerization capacity and therefore pore formation activity of the insecticidal protein affinity-bound by the affinity construct would be enhanced through higher binding capacities to insect receptors via the multispecific affinity construct.

Alternatively, the multispecific affinity constructs can be easily expressed in plants either alone or together (i.e., by co-expression) with the insecticidal protein. Affinity molecules such as the V_{H}Hs can be readily expressed by transformed plants (Ismaili et al. 2007, Biotechnol Appl Biochem 47, 11-19). Expression in transgenic plants can be done using constitutively active promotors (e.g., 35S promotor or Ubiquitin promotors) or using specific promotors that allow increasing toxin activity in areas that are attacked by the target insects or that can be induced via external cues (e.g., chemically-inducible promotors, heat-inducible promotors).

The invention also includes applying the insecticidal protein to which the affinity molecule B is binding to or is directed to (or intended to bind to) to the plant. The insecticidal protein that is co-applied with the affinity molecule might also be equipped with a tag that is specific for a V_{H}H. Such tags have been described previously (De Genst et al. 2010, J Mol Biol 402, 326-343). However, these tags could be any protein or amino acid sequence, for which a specific antibody or V_{H}H can be produced.

The multispecific affinity constructs can also be introduced to the plant by other means, such as viral vectors, bacteria, injection, grafting, spraying and others.

### Generation of Antibodies, in Particular Single Domain Antibodies, and Alphabodies, Nanobodies and CDR3-Loops

Once insect-specific target structures are identified and made available, they can be used to immunize mammals, for example, camelids, as one of the steps to gain affinity molecules of the present invention that bind these insect-specific target structures (see FIG. 4). As a response to immunization with an antigen camelid produce antibodies consisting of two heavy chains and two light chains, but also antibodies consisting of the variable domain of the heavy chain. After the immunization and an optional booster injection, mRNA from white blood cells is produced. The mRNA in its entirety is screened for the mRNA of heavy chain antibodies by reverse transcription and PCR methods. A library consisting of the single domain antibodies with a multitude of clones is therefore generated. In a subsequent screening step phage display or ribosome display is used to isolate antigen binding clones. As an alternative, sharks can be used to generate VNAR (Variable New Antigen Receptor) fragments. Antibodies generated with this technology may be subject to affinity maturation to further increase the antibody affinity. Alternatively, single domain antibodies of the present disclosure may be produced using naïve gene libraries from animals that have not been immunized. Also, single domain antibodies according to the present disclosure may be made from common murine or human IgG having four chains in a similar manner, using gene libraries from immunized or naïve donors and display techniques for the identification of the most specific antigens. Phage display is the most popular method for antibody library generation, including the generation of camelid single domain antibody libraries.

Antibodies for use in the affinity constructs of the present disclosure can also be generated by using in silico methodologies which are known one of ordinary skill in the art.

In silico methodologies are also applied with regard to the generation of alphabodies according to the present disclosure. The alphabody scaffold is a computationally designed protein scaffold of about 10 kDa molecular weight and is considered not to have a counterpart in nature. Alphabodies can carry up to 25 variable positions that can be optimized, inter alia, for binding properties. This offers important advantages in the targeting of receptor molecules according to the present disclosure. Alphabodies can be considered as one of the preferred affinity molecules of the present disclosure.

The alphabodies and antibodies, in particular single domain antibodies and fragments thereof, obtained can then be fused or coupled to form an affinity construct of the disclosure, optionally via a linker L as described herein.

### Microbial Strains for Use as Host Cells

One aspect of the disclosure pertains to microbial strains that are capable of expressing the novel affinity constructs of the present disclosure. In one embodiment, microbial strains can also be used for producing the novel affinity constructs of the present disclosure comprising at least one affinity molecule A capable of recognizing, or capable of binding to, or binding to, or being directed to, or being designed to bind to an insect-specific structure in and/or on a target insect, and at least one affinity molecule B capable of binding to, or binding to, or being directed to, or being designed to bind to an insecticidal protein (toxin).

The disclosure encompasses a method for expressing in a microbial cell, preferably in a bacterial cell, a yeast cell or a fungal cell, a novel affinity constructs of the disclosure, comprising the steps of: (a) inserting into a microbial cell, preferably into a bacterial cell, a yeast cell or a fungal cell, a nucleic acid sequence comprising in 5′ to 3′ direction an operably linked recombinant, double-stranded DNA molecule, wherein the recombinant double-stranded DNA molecule comprises (i) a promoter that functions in the microbial cell; (ii) a nucleic acid molecule encoding a affinity construct of the disclosure; and (iii) a 3′ non-translated polynucleotide that functions in the microbial cell to cause termination of transcription; (b) obtaining a transformed microbial cell comprising the nucleic acid sequence of step (a) capable of expressing an affinity construct of the disclosure. The disclosure encompasses a microbial cell produced by such a method. The affinity constructs so produced or the microbial cells as such (still comprising the affinity constructs of the present disclosure) can be used in compositions of the present disclosure, in particular in spray compositions provided by the present disclosure. In preferred embodiments, the composition or the spray is applied to the plants, preferably to the leaves or other plant parts where insect pests generally feed, or to plant seeds. The term “microbial strain” or “microbial cell” or “microorganism” as used herein encompasses prokaryotic and eukaryotic microbes. Preferably, the microbial cell is any one of a bacterial cell, a yeast cell or a fungal cell. More preferably, the microbial cell is an endophyte, such as, for example, a bacterial or fungal cell. Even more preferred, the microbial cell is a bacterial cell. Preferably, the bacterial cell is E. coli.

### Nucleic Acid Molecules, and Variants and Fragments Thereof

Another aspect of the disclosure pertains to recombinant nucleic acid molecules comprising nucleic acid sequences encoding the novel affinity constructs of the present disclosure or biologically active portions thereof.

As used herein, the term “nucleic acid molecule” is intended to include DNA molecules (e.g., recombinant DNA, cDNA, genomic DNA, plastid DNA, mitochondrial DNA) and RNA molecules (e.g., mRNA) and analogs of the DNA or RNA generated using nucleotide analogs. The nucleic acid molecule can be single-stranded or double-stranded, but preferably is double-stranded DNA.

As used herein, an “isolated” nucleic acid molecule (or DNA) refers to a nucleic acid sequence (or DNA) that is no longer in its natural environment, for example in vitro.

As used herein, a “recombinant” nucleic acid molecule (or DNA) refers to a nucleic acid sequence (or DNA) that is in a recombinant bacterial or plant host cell.

In various embodiments, an isolated nucleic acid molecule encoding an insecticidal protein, which forms part of a composition of the disclosure comprising the novel affinity constructs of the present invention and an insecticidal protein (toxin), has one or more changes in the nucleic acid sequence compared to the native or genomic nucleic acid sequence. In various embodiments, the change in the native or genomic nucleic acid sequence includes, but is not limited to: changes in the nucleic acid sequence due to the degeneracy of the genetic code; changes in the nucleic acid sequence due to amino acid substitution, insertion, deletion and/or addition compared to the native or genomic sequence; removal of one or more introns; deletion of one or more upstream or downstream regulatory regions; and deletion of the 5′ and/or 3′ untranslated region associated with the genomic nucleic acid sequence. In various embodiments, the nucleic acid molecule encoding an insecticidal protein, which forms part of a composition of the disclosure comprising the novel affinity constructs and an insecticidal protein (toxin), is a non-genomic sequence.

A variety of polynucleotides that encode affinity constructs of the present disclosure or related proteins are contemplated. Such polynucleotides are useful for production of the novel affinity constructs in host cells, such as, for example, plant cells or bacterial (microbial) cells, when operably linked to suitable promoter, transcription termination and/or polyadenylation sequences.

Where appropriate, a nucleic acid may be optimized for increased expression in the host cell organism. Thus, where the host organism is a plant, the synthetic nucleic acids can be synthesized using plant-preferred codons for improved expression. See, for example, Campbell and Gowri 1990; Plant Physiol. 92:1-11 for a discussion of host-preferred codon usage. For example, although nucleic acid sequences of the disclosure may be expressed in both monocotyledonous and dicotyledonous plant species, sequences can be modified to account for the specific codon preferences and GC content preferences of monocotyledons or dicotyledons as these preferences have been shown to differ (Murray et al. 1989; Nucleic Acids Res. 17:477-498). Thus, for example the maize-preferred codon for a particular amino acid may be derived from known gene sequences from maize. Methods are available in the art for synthesizing plant-preferred genes. See, for example, Murray, et al. 1989; Nucleic Acids Res. 17:477-498, and Liu H et al. 2010; Mol Bio Rep 37:677-684. Additional sequence modifications are known to enhance gene expression in a cellular host. These include elimination of sequences encoding spurious polyadenylation signals, exon-intron splice site signals, transposon-like repeats, and other well-characterized sequences that may be deleterious to gene expression. The GC content of the sequence may be adjusted to levels average for a given cellular host, as calculated by reference to known genes expressed in the host cell. The term “host cell” as used herein refers to a cell which contains a vector and supports the replication and/or expression of the expression vector is intended. Host cells may be prokaryotic cells, such as E. coli, or eukaryotic cells, such as yeast or insect cells or monocotyledonous or dicotyledonous plant cells. An example of a monocotyledonous host cell is a maize host cell.

Polynucleotides that encode an affinity construct of the present disclosure can also be synthesized de novo from a corresponding polypeptide sequence. The sequence of the polynucleotide gene can be deduced from a polypeptide sequence through use of the genetic code. Computer programs such as “BackTranslate” (GCG™ Package, Acclerys, Inc. San Diego, Calif.) can be used to convert a peptide sequence to the corresponding nucleotide sequence encoding the peptide.

Furthermore, synthetic polynucleotide sequences of the disclosure, which encode an affinity construct of the present disclosure, can be designed (for example using codon optimization, which is well known to the skilled person) so that they will be expressed in plants or microbial cells. Methods for synthesizing plant genes to improve the expression level of the protein encoded by the synthesized gene are known in the art. Such methods include the modification of the structural gene sequences of the exogenous transgene, to cause them to be more efficiently transcribed, processed, translated and expressed by the plant. Features of genes that are expressed well in plants include elimination of sequences that can cause undesired intron splicing or polyadenylation in the coding region of a gene transcript while retaining substantially the amino acid sequence of the toxic portion of the insecticidal protein.

A method for obtaining enhanced expression of transgenes in monocotyledonous plants is disclosed, e.g., in U.S. Pat. No. 5,689,052.

Also provided are nucleic acid molecules that encode transcription and/or translation products that are subsequently spliced to ultimately produce a functional insecticidal fusion protein of the present disclosure. Splicing can be accomplished in vitro or in vivo and can involve cis- or trans-splicing. The substrate for splicing can be polynucleotides (e.g., RNA transcripts) or polypeptides. An example of cis-splicing of a polynucleotide is where an intron inserted into a coding sequence is removed and the two flanking exon regions are spliced to generate an insecticidal protein encoding sequence, or an (insecticidal) fusion protein encoding sequence. An example of trans-splicing would be where a polynucleotide is encrypted by separating the coding sequence into two or more fragments that can be separately transcribed and then spliced to form the full-length insecticidal protein encoding sequence, or an insecticidal fusion protein encoding sequence. Thus, in various embodiments the polynucleotides do not directly encode a full-length insecticidal protein or insecticidal fusion protein, but rather encode a fragment or fragments thereof. These polynucleotides can be used to express a functional affinity construct through a mechanism involving splicing, where splicing can occur at the level of polynucleotide (e.g., intron/exon) and/or polypeptide (e.g., intein/extein). This can be useful, for example, in controlling expression of insecticidal activity, in particular in case functional affinity constructs may only be expressed if all required fragments are expressed in an environment that permits splicing processes to generate functional product.

Nucleic acid molecules that are fragments of the nucleic acid sequences encoding affinity constructs of the present disclosure are also encompassed herein. By “fragment” is intended a portion of the nucleic acid sequence encoding an affinity construct of the disclosure. A fragment of a nucleic acid sequence may encode a biologically active portion of an affinity construct of the disclosure, or it may be a fragment that can be used as a hybridization probe or PCR primer. Nucleic acid molecules that are fragments of a nucleic acid sequence encoding an affinity construct of the disclosure comprise at least about 50, 100, 200, 300, 400, 500, 600, 700, 800, 900, 1,000, 1,100, 1,200, 1,300, 1,400, or 1,500 contiguous nucleotides or up to the number of nucleotides present in a full-length nucleic acid sequence encoding an affinity construct of the disclosure, depending upon the intended use. By “contiguous” nucleotide residues are intended that are immediately adjacent to one another. Fragments of the nucleic acid sequences of the disclosure will encode protein fragments that retain the biological activity of the corresponding affinity construct of the disclosure and, hence, retain its ability to bind to one or more insect-specific structure(s) in or on the insect pest as well as to bind one or more insecticidal protein (toxin).

As used herein, the term “insecticidal activity” refers to the activity of a composition of the disclosure comprising the novel affinity construct and an insecticidal protein (toxin), that can be measured by, but is not limited to, mortality, weight loss, stunted growth of the insect pest and other behavioral and physical changes of an insect pest after feeding and exposure for an appropriate length of time. Thus, an organism or substance having insecticidal activity adversely impacts at least one measurable parameter of insect pest fitness. For example, “insecticidal proteins” are proteins that display insecticidal activity by themselves but may also display insecticidal activity in combination with other proteins. The same holds for the insecticidal activity of a composition of the disclosure comprising the novel affinity construct and an insecticidal protein (toxin), which displays insecticidal activity by itself, but may also display insecticidal activity in combination with other proteins.

As used herein, the term “insecticidally effective amount” connotes a quantity of an insecticidal protein applied in combination with the affinity construct of the disclosure that has insecticidal activity when present in the environment of a pest. For each insecticidal protein, the insecticidally effective amount is determined empirically for each insect pest affected in a specific environment.

By “retains activity” is intended that an insecticidal protein has at least about 10%, at least about 30%, at least about 50%, at least about 70%, 80%, 90%, 95% or higher of the insecticidal activity compared to the full-length insecticidal protein alone that is part of the composition of the present invention. In various preferred embodiments, the insecticidal activity is against an Isopteran, Blattodean, Orthopteran, Phthirapteran, Thysanopteran, Hemipteran, Hymenopteran, Siphonapteran, Dipteran, Coleopteran and/or Lepidopteran species. In various preferred embodiments, the insecticidal activity is against a Lepidopteran species. In further preferred embodiments, the insecticidal activity is against a Coleopteran species.

In some embodiments a fragment of a nucleic acid sequence encoding a biologically active portion of an insecticidal protein will encode at least about 15, 25, 30, 50, 75, 100, 125, 150, 175, 200 or 250, contiguous amino acids present in a full-length insecticidal protein.

In various embodiments, the insecticidal protein, which forms part of the composition of the disclosure comprising the novel affinity construct and an insecticidal protein (toxin), may be the core of an insecticidal toxin, preferably the core of a Cry toxin, more preferably the core of a three-domain Cry protein. Thus, in various embodiments, the insecticidal protein is a core toxin, preferably a core toxin of a Cry toxin, more preferably the core of a three-domain Cry protein. The “core” of an insecticidal Cry toxin is a fragment of the insecticidal toxin and may comprise domains I, II and/or III of the insecticidal Cry toxin. A core toxin according to the present disclosure has insecticidal activity as described herein elsewhere.

In various embodiments, an insecticidal protein, which forms part of the composition of the disclosure comprising the novel affinity construct and an insecticidal protein (toxin), has an amino acid sequence comprising at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence of the Cry1Ac 3 domain core toxin (SEQ ID NOS. 51), wherein the insecticidal protein has insecticidal activity. In various embodiments, an insecticidal protein, which forms part of the insecticidal composition of the disclosure comprising the novel affinity construct and an insecticidal protein (toxin), has an amino acid sequence comprising at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence of the Cry3Ab 3 domain core toxin (SEQ ID NOS. 52), wherein the insecticidal protein has insecticidal activity. In various embodiments, an insecticidal protein, which forms part of the composition of the disclosure comprising the novel affinity construct and an insecticidal protein (toxin), has an amino acid sequence comprising at least 80%, 81%, 82%, 83%, 84%, 85%, 86%, 87%, 88%, 89%, 90%, 91%, 92%, 93%, 94%, 95%, 96%, 97%, 98%, 99% or greater identity to the amino acid sequence of the Vip3Aa toxin (SEQ ID NOS. 53), wherein the insecticidal protein has insecticidal activity.

The “sequence identity” is intended to refer to an amino acid or nucleic acid sequence that has at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to a reference sequence using an alignment program known in the art using standard parameters. In various embodiments the sequence homology/identity is against the full-length sequence of a reference insecticidal protein of the disclosure.

One of skill in the art will recognize that these values can be appropriately adjusted to determine corresponding identity of proteins encoded by two nucleic acid sequences by taking into account codon degeneracy, amino acid similarity, reading frame positioning, and the like. To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences have the same length. In another embodiment, the comparison is across the entirety of the reference sequence. The percent identity between two sequences can be determined using techniques similar to those described below, with or without allowing gaps. In calculating percent identity, typically exact matches are counted. The determination of percent identity between two sequences can be accomplished using a mathematical algorithm. A non-limiting example of a mathematical algorithm utilized for the comparison of two sequences is the algorithm of Karlin and Altschul 1990; Proc. Natl. Acad. Sci. USA 87:2264, modified as in Karlin and Altschul 1993; Proc. Natl. Acad. Sci. USA 90:5873-5877. Such an algorithm is incorporated into the BLASTN and BLASTX programs of Altschul, et al. 1990; J. Mol. Biol. 215:403. BLAST nucleotide searches can be performed with the BLASTN program, score=100, word length=12, to obtain nucleic acid sequences homologous to insecticidal-like nucleic acid molecules. BLAST protein searches can be performed with the BLASTX program, score=50, word length=3, to obtain amino acid sequences homologous to insecticidal protein molecules. To obtain gapped alignments for comparison purposes, Gapped BLAST (in BLAST 2.0) can be utilized as described in Altschul, et al. 1997; Nucleic Acids Res. 25:3389. Alternatively, PSI-Blast can be used to perform an iterated search that detects distant relationships between molecules. See, Altschul, et al., (1997), supra. When utilizing BLAST, Gapped BLAST, and PSI-Blast programs, the default parameters of the respective programs (e.g., BLASTX and BLASTN) can be used. Alignment may also be performed manually by inspection.

The present disclosure also encompasses nucleic acid molecules encoding variants of either the affinity constructs of the disclosure or the insecticidal protein (toxin) used in the context of the present invention. These “variants” include sequences that encode the affinity constructs of the disclosure or the insecticidal protein (toxin) used in the context of the present invention but that differ conservatively because of the degeneracy of the genetic code as well as those that are sufficiently identical as discussed above. Variant nucleic acid sequences also include synthetically derived nucleic acid sequences that have been generated, for example, by using site-directed mutagenesis but which still encode an (insecticidal) fusion protein of the present disclosure. The present disclosure provides isolated or recombinant polynucleotides that encode any of the affinity constructs or the insecticidal protein (toxin) disclosed herein. Those having ordinary skill in the art will readily appreciate that due to the degeneracy of the genetic code, a multitude of nucleotide sequences encoding either the affinity constructs of the disclosure or the insecticidal protein (toxin) used in the context of the present disclosure exist. The skilled artisan will further appreciate that changes can be introduced by mutation of the nucleic acid sequences thereby leading to changes in the amino acid sequence of the encoded affinity constructs of the disclosure or the insecticidal protein (toxin) used in the context of the present invention, without altering the biological activity of the proteins. Thus, variant nucleic acid molecules can be created by introducing one or more nucleotide substitutions, additions or deletions into the corresponding nucleic acid sequence disclosed herein, such that one or more amino acid substitutions, additions or deletions are introduced into the encoded protein. Mutations can be introduced by standard techniques, such as site-directed mutagenesis and PCR-mediated mutagenesis. Mutations may also be introduced using genome editing tools like, e.g., Zinc Finger Nucleases, TAL Effector Nucleases (TALEN), and CRISPR/Cas systems, like, for example, CRISPR/Cas9 and CRISPR/cpf1. Such variant nucleic acid sequences are also encompassed by the present disclosure.

### Proteins and Variants and Fragments Thereof

Insecticidal proteins or polypeptides are encompassed by the present disclosure. By “insecticidal protein”, or “insecticidal polypeptide”, is intended a protein or polypeptide that retains insecticidal activity against one or more insect pests of, e.g., the Isopteran, Blattodean, Orthopteran, Phthirapteran, Thysanopteran, Hemipteran, Hymenopteran, Siphonapteran, Dipteran, Coleopteran and/or Lepidopteran order. A variety of insecticidal proteins/polypeptides are contemplated.

As used herein, the terms “protein”, “peptide molecule” or “polypeptide” includes any molecule that comprises five or more amino acids. It is well known in the art that protein, peptide or polypeptide molecules may undergo modification, including post-translational modifications, such as, but not limited to, disulfide bond formation, glycosylation, phosphorylation or oligomerization. Thus, as used herein, the terms “protein”, “peptide molecule” or “polypeptide” includes any protein that is modified by any biological or non-biological process. The terms “amino acid” and “amino acids” refer to all naturally occurring L-amino acids.

A “recombinant protein” is used herein to refer to a protein that is no longer in its natural environment, for example in vitro or in a recombinant bacterial or plant host cell.

In various embodiments of the present disclosure, a fragment of an affinity molecule or antibody as disclosed herein means antigen-binding fragment of an affinity molecule or antigen-binding fragment of an antibody.

In the present disclosure, the terms “fragment”, “variant”, “derivative” and “analog” when referring to affinity molecules, in particular antibodies, more specifically single domain antibodies, include any “fragment”, “variant”, “derivative” and “analog” which retain at least some of the affinity properties of the corresponding native affinity molecule. Thus, when referring to antibodies as affinity molecules, in particular single domain antibodies, the terms “fragment”, “variant”, “derivative” and “analog” describe polypeptide fragments, variants or derivatives, which retain at least some of the antigen-binding properties of the corresponding native antibodies. Thus, polypeptide fragments may also be considered as “biologically active portions” of a polypeptide.

Fragments of affinity molecules (referring to both affinity molecules A and B) of the present disclosure, in particular fragments of polypeptides including fragments of antibodies, include proteolytic fragments, as well as deletion fragments, in addition to specific antibody fragments discussed elsewhere herein. Variants of antibodies and antibody polypeptides of the present disclosure include fragments as described above, and also polypeptides and antibody polypeptides with altered amino acid sequences due to amino acid substitutions, deletions, or insertions. Variants may occur naturally or may be non-naturally occurring. Non-naturally occurring variants may be produced using art-known mutagenesis techniques. Variant polypeptides and antibody polypeptides may comprise conservative or non-conservative amino acid substitutions, deletions or additions. Derivatives of antibodies and antibody polypeptides of the present disclosure are polypeptides, which have been altered so as to exhibit additional features not found on the native polypeptide or antibody polypeptide. Examples include, but are not limited to, fusion proteins. Variant polypeptides may also be referred to herein as “polypeptide analogs”. As used herein, a “derivative” of an antibody or antibody polypeptide refers to a subject polypeptide or antibody polypeptide having one or more residues chemically derivatized by reaction of a functional side group. Also included as “derivatives” are those peptides, which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

“Fragments” or “biologically active portions” include polypeptide fragments comprising amino acid sequences, which are sufficiently identical to an insecticidal protein disclosed herein that exhibits insecticidal activity. A biologically active portion of an insecticidal protein disclosed herein can be a polypeptide that is, for example, 10, 25, 50, 100, 150, 200, 250 or more amino acids in length. Such biologically active portions can be prepared by recombinant techniques and evaluated for insecticidal activity.

It is well known in the art that polynucleotides encoding a truncated insecticidal protein disclosed herein can be engineered to add a start codon at the N-terminus such as ATG encoding methionine. It is also well known in the art that depending on the host in which the insecticidal protein disclosed herein is expressed the methionine may be partially or completed processed off.

The term variants also refers to proteins or polypeptides having an amino acid sequence that is at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the parental amino acid sequence.

In some embodiments an insecticidal protein disclosed herein includes variants where an amino acid that is part of a proteolytic cleavage site is changed to another amino acid to eliminate or alter the proteolytic cleavage at that site. This is in particular relevant when protoxins are used as insecticidal proteins in the context of the present disclosure and as discussed elsewhere herein. In some embodiments the proteolytic cleavage is caused by a protease in the insect gut. In other embodiments the proteolytic cleavage is caused by a plant protease in the transgenic plant.

In various embodiments an insecticidal protein disclosed herein has a modified physical property. As used herein, the term “physical property” refers to any parameter suitable for describing the physico-chemical characteristics of a protein. As used herein, “physical property of interest” and “property of interest” are used interchangeably to refer to physical properties of proteins that are being investigated and/or modified. Examples of physical properties include, but are not limited to, net surface charge and charge distribution on the protein surface, net hydrophobicity and hydrophobic residue distribution on the protein surface, surface charge density, surface hydrophobicity density, total count of surface ionizable groups, surface tension, protein size and its distribution in solution, melting temperature, and heat capacity. Examples of physical properties also include, but are not limited to, solubility, folding, stability, in particular pH stability, and digestibility. In various embodiments an insecticidal fusion protein of the disclosure has increased digestibility of proteolytic fragments in an insect gut. Models for digestion by simulated gastric fluids are known to one skilled in the art (Fuchs, R. L. and J. D. Astwood 1996; Food Technology 50: 83-88; Astwood, J. D., et al. 1996; Nature Biotechnology 14: 1269-1273; Fu T J et al. 2002; J. Agric Food Chem. 50: 7154-7160).

Also described herein are means and methods that provide for stability of the affinity construct and the affinity molecules, respectively, such as, for example, single domain antibodies in insect digestive systems. The affinity construct and the affinity molecules of the present disclosure may be degraded by the action of digestive enzymes, including proteases in the insect digestive system. To decrease potential proteolysis of the affinity construct and the affinity molecules in the insect digestive system, the amino acid composition may be changed without changing the binding capacity of the affinity molecules.

As described herein, the single domain antibody or a fragment thereof, e.g., the CDR3 loop of an sdAb, may be modified to provide for stability against proteases, such as the introduction of Cys at selected positions to form an extra disulfide bond. Phage-display panning with single domain antibodies can be performed under conditions that mimic the harsh environment of insect (mid)guts (e.g., pH>9). Other modifications providing for resistance against enzymatic proteolysis are described in the art and are known to the skilled person.

Bacterial genes quite often possess multiple methionine initiation codons in proximity to the start of the open reading frame. Often, translation initiation at one or more of these start codons will lead to generation of a functional protein. These start codons can include ATG codons. However, bacteria such as Bacillus sp. also recognize the codon GTG as a start codon, and proteins that initiate translation at GTG codons contain a methionine at the first amino acid. On rare occasions, translation in bacterial systems can initiate at a TTG codon, though in this event the TTG encodes a methionine. Furthermore, it is not often determined a priori which of these codons are used naturally in the bacterium. Thus, it is understood that use of one of the alternate methionine codons may also lead to the generation of insecticidal proteins. Corresponding insecticidal proteins are encompassed in the present disclosure and may be used in the methods of the present disclosure. It will be understood that, when expressed in plants, it will be necessary to alter the alternate start codon to ATG for proper translation.

In another aspect, the affinity constructs of the disclosure may be expressed as a precursor protein with an intervening sequence that catalyzes multi-step, post-translational protein splicing. Protein splicing involves the excision of an intervening sequence from a polypeptide with the concomitant joining of the flanking sequences to yield a new polypeptide.

Polynucleotides encoding an affinity construct of the disclosure may be fused to signal sequences, which will direct the localization of the affinity construct to particular compartments of a prokaryotic or eukaryotic cell and/or direct the secretion of the affinity construct of the disclosure from a prokaryotic or eukaryotic cell. For example, in E. coli, one may wish to direct the expression of the affinity construct to the periplasmic space. Further examples of compartments of the plant cell in this regard are chloroplasts, the Golgi apparatus, mitochondria, the nucleus, the endoplasmatic reticulum but also targeting of the extracellular space, targeting of the symplast or the cell wall.

### Nucleotide Constructs, Expression Cassettes and Vectors

The use of the term “nucleotide constructs” herein is not intended to limit the embodiments to nucleotide constructs comprising DNA. Those of ordinary skill in the art will recognize that nucleotide constructs, particularly polynucleotides and oligonucleotides, composed of ribonucleotides and combinations of ribonucleotides and deoxyribonucleotides may also be employed as disclosed herein. The nucleotide constructs, nucleic acids, and nucleotide sequences as described herein encompass all complementary forms of such constructs, molecules and sequences. Further, the nucleotide constructs, nucleotide molecules and nucleotide sequences of the disclosure encompass all nucleotide constructs, molecules and sequences, which can be employed in the methods of the disclosure for transforming plants and microorganisms including, but not limited to, those comprised of deoxyribonucleotides, ribonucleotides and combinations thereof. Such deoxyribonucleotides and ribonucleotides include both naturally occurring molecules and synthetic analogues. The nucleotide constructs, nucleic acids, and nucleotide sequences of the disclosure also encompass all forms of nucleotide constructs including, but not limited to, single-stranded forms, double-stranded forms, hairpins, stem-and-loop structures and the like.

A further embodiment relates to a transformed organism such as an organism selected from bacterial or eukaryotic cells. The transformed organism comprises a DNA molecule of the disclosure, an expression cassette comprising the DNA molecule or a vector comprising the expression cassette, which may be stably incorporated into the genome of the transformed organism.

The sequences of the disclosure are provided in DNA constructs for expression in the organism of interest. In various embodiments, the sequences of the disclosure are provided in expression cassettes. The disclosure encompasses an expression cassette comprising an isolated nucleic acid molecule encoding an affinity construct or insecticidal protein of the disclosure. An “expression cassette” as used herein means a DNA construct comprising at least one regulatory sequence operably linked to a polynucleotide encoding an affinity construct of the disclosure. The term “operably linked” as used herein refers to a functional linkage between a promoter and a DNA sequence, wherein the promoter sequence initiates and mediates transcription of the DNA sequence. Generally, “operably linked” means that the nucleic acid sequences being linked are contiguous to join two protein coding regions in the same reading frame. In various embodiments the expression cassette comprises a 5′ and a 3′ regulatory sequence. In some embodiments the expression cassette comprises a heterologous regulatory sequence. The term “heterologous regulatory sequence” as used herein indicates that the regulatory sequence is not associated with the native or genomic polynucleotide encoding an affinity construct or insecticidal protein of the disclosure. In some embodiments, the expression cassette comprises a regulatory sequence from a plant. In some embodiments the expression cassette comprises a regulatory sequence from the bacterial strain which is used as host for the expression and production of the novel affinity construct and insecticidal proteins of the present disclosure. The expression and production of the novel affinity construct and insecticidal proteins of the present disclosure is discussed elsewhere herein. The construct may additionally contain at least one additional gene to be co-transformed into the organism. Alternatively, the additional gene(s) can be provided on multiple DNA constructs. Such a DNA construct is provided with a plurality of restriction sites for insertion of the nucleotide sequence encoding the affinity construct or insecticidal protein to be under the transcriptional regulation of the regulatory regions. The DNA construct may additionally contain selectable marker genes. An expression cassette will generally include in the 5′ to 3′ direction of transcription: a transcriptional and translational initiation region (i.e., a promoter), a DNA sequence of the embodiments and a transcriptional and translational termination region (i.e., termination region) functional in the organism serving as a host. The transcriptional initiation region (i.e., the promoter) may be native, analogous, foreign or heterologous to the host organism and/or to the sequence of the embodiments. Additionally, the promoter may be the natural sequence or alternatively a synthetic sequence. The term “foreign” as used herein indicates that the promoter is not found in the native organism into which the promoter is introduced. Where the promoter is “foreign” or “heterologous” to the sequence(s) of the disclosure, it is intended that the promoter is not the native or naturally occurring promoter for the operably linked sequence(s) of the disclosure. Where the promoter is a native or natural sequence, the expression of the operably linked sequence is altered from the wild-type expression, which results in an alteration in phenotype. In various embodiments the expression cassette may also include a transcriptional enhancer sequence. As used herein, the term an “enhancer” refers to a DNA sequence, which can stimulate promoter activity and may be an innate element of the promoter or a heterologous element inserted to enhance the level or tissue-specificity of a promoter.

The expression cassettes may additionally contain 5′ leader sequences. Such leader sequences can act to enhance translation. Translation leaders are known in the art. Such constructs may also contain a “signal sequence” or “leader sequence” to facilitate co-translational or post-translational transport of the peptide to certain intracellular structures such as the chloroplast (or other plastid), endoplasmic reticulum or Golgi apparatus. By “signal sequence” is intended a sequence that is known or suspected to result in co-translational or post-translational peptide transport across the cell membrane. In eukaryotes, this typically involves secretion into the Golgi apparatus, with some resulting glycosylation. Insecticidal toxins of bacteria are often synthesized as protoxins, which are proteolytically activated in the gut of the target pest (Chang 1987; Methods Enzymol. 153:507-516). In various embodiments, the signal sequence is located in the native sequence or may be derived from a sequence of the embodiments. By “leader sequence” is intended any sequence that when translated, results in an amino acid sequence sufficient to trigger co-translational transport of the peptide chain to a subcellular organelle. Thus, this includes leader sequences targeting transport and/or glycosylation by passage into the endoplasmic reticulum, passage to vacuoles, plastids including chloroplasts, mitochondria and the like. In preparing the expression cassette, the various DNA fragments may be manipulated so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, re-substitutions, e.g., transitions and transversions, may be involved. A number of promoters can be used in the practice of the present disclosure. The promoters can be selected based on the desired outcome. The nucleic acids can be combined with constitutive, tissue-preferred, inducible or other promoters for expression in the host organism. Suitable constitutive promoters for use in a plant host cell are known in the art.

Depending on the desired outcome, it may be beneficial to express the gene from an inducible promoter. Of particular interest for regulating the expression of the nucleotide sequences of the disclosure in plants are wound-inducible promoters. Additionally, pathogen-inducible promoters may be employed in the methods of the disclosure and nucleotide constructs of the disclosure. Tissue-preferred promoters can be utilized to target enhanced insecticidal fusion protein expression within a particular plant tissue. Leaf-preferred promoters are known in the art and are also encompassed by the present disclosure. Root-preferred or root-specific promoters are also encompassed and are known and can be selected from the many available from the literature or isolated de novo from various compatible species. “Seed-preferred” promoters include both “seed-specific” promoters (those promoters active during seed development such as promoters of seed storage proteins) as well as “seed-germinating” promoters (those promoters active during seed germination). See, Thompson, et al., 1989; BioEssays 10:108.

Where low level expression is desired, weak promoters will be used. Generally, the term “weak promoter” as used herein refers to a promoter that drives expression of a coding sequence at a low level. The above list of promoters is not meant to be limiting. Any appropriate promoter can be used in the embodiments.

Generally, the expression cassette may comprise a selectable marker gene for the selection of transformed cells. Selectable marker genes are utilized for the selection of transformed cells or tissues. Marker genes include, but are not limited to, genes encoding antibiotic resistance, as well as genes conferring resistance to herbicidal compounds. Any selectable marker gene can be used in the present disclosure.

The disclosure encompasses a recombinant microorganism, comprising an isolated nucleic acid molecule encoding an affinity construct or an insecticidal protein of the disclosure. In various embodiments, the microorganism is any of a bacterium, baculovirus, algae, and fungi. In various embodiments, the microorganism is any of a Bacillus, a Pseudomonas, a Clavibacter, a Rhizobium, a lactobacillus or E. coli. Preferably, the recombinant microorganism is Bacillus thuringiensis or Escherichia coli or a expression system like, for example, Saccharomyces cerevisiae or Pichia pastoris.

The disclosure encompasses a method for producing an affinity construct or an insecticidal protein of the disclosure, comprising culturing a microorganism of the disclosure under conditions in which the nucleic acid molecule encoding the affinity construct and/or insecticidal protein, respectively, is expressed. Preferably, the affinity construct and/or insecticidal protein of the present disclosure is either secreted by the microorganism into the culture medium and collected or isolated therefrom, or it is extracted from the microorganism after a period of culture and then formulated into an (insecticidal) composition/formulation according to the present disclosure. The protein can be collected or purified by using a tag, for example, a histidine tag. The most commonly used tag for collecting large amounts of highly purified protein is a poly-histidine tag (His-tag). His-tagged proteins are recombinant proteins designed to include a poly-histidine tail (his-tag) that facilitates purification of the proteins from in vitro expression systems, e.g., from bacterial host strains used for expression of the proteins. The His-tag usually comprises 6-14 histidines and is typically fused to the N- or C-terminal end of a target protein. In some cases, the tag can also be inserted into an exposed loop of the target protein. His-tagged insecticidal fusion proteins and fusion proteins of the present disclosure expressed and subsequently purified by a purification kit for histidine-tagged proteins (such purification kits are commercially available, e.g., from Qiagen or Sigma) are suitable for subsequent use in a composition or formulation, preferably a spray composition or formulation, according to the present disclosure. The disclosure also encompasses a method for producing a microorganism that contains an affinity construct and/or an insecticidal protein, respectively, of the disclosure comprising culturing a microorganism of the disclosure under conditions in which the nucleic acid molecule encoding the affinity construct and/or insecticidal protein, respectively, is expressed, collecting or isolating the microorganism from the culture medium after a period of culture and then formulating the microorganism into an (insecticidal) composition or formulation according to the present disclosure.

### Plant Transformation

The novel affinity constructs and/or the insecticidal proteins of the present disclosure can be easily expressed in transgenic plants or in plant cells or be applied as an insecticidal spray/solution to a plant, seed or insect, in particular in agricultural crops or cells thereof. Furthermore, the affinity construct and/or insecticidal protein, respectively, can be readily expressed by transformed plants or plant cells. Also, the novel affinity constructs of the present disclosure can be easily co-expressed with an insecticidal protein in transgenic plants or in plant cells, or can be applied, in combination with an insecticidal protein, as an insecticidal spray/solution to a plant, seed or insect, in particular in agricultural crops or cells thereof, wherein the insecticidal protein corresponds to the insecticidal protein (toxin), which the at least one affinity molecule B of the novel affinity construct is capable of binding to, or is binding to, or is being directed to, or is being designed to bind to.

The present disclosure provides a plant, plant part or plant seed comprising (i) one or more nucleic acid sequences encoding a novel affinity construct of the present disclosure and an insecticidal protein, wherein the insecticidal protein corresponds to the insecticidal protein, against which the at least one of the affinity molecules of the novel affinity construct is binding to, or (i) one or more vectors comprising one or more nucleic acid sequences encoding a novel affinity construct of the present disclosure and an insecticidal protein, wherein the insecticidal protein corresponds to the insecticidal protein (toxin), which the at least one affinity molecule B of the novel affinity construct is capable of binding to, or binding to, or being directed to, or being designed to bind to. The plant may be a monocotyledonous plant or a dicotyledonous plant. The present disclosure also provides parts and seed of such plants.

The methods of the present disclosure involve introducing an affinity construct or a polynucleotide encoding same into a plant. “Introducing” is intended to mean presenting to a plant cell or plant the affinity construct or the polynucleotide encoding same in such a manner that the sequence gains access to the interior of a cell of the plant. The methods of the disclosure do not depend on a particular method for introducing a polynucleotide or polypeptide into a plant, what is relevant is that the polynucleotide or polypeptides gains access to the interior of at least one cell of the plant. Methods for introducing polynucleotide or polypeptides into plants are well known in the art including, but not limited to, stable transformation methods, transient transformation methods and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant. The term “plant” comprises whole plants, plant organs or plant parts (e.g., leaves, stems, roots, etc.), seeds, plant cells, propagules, embryos and progeny of the same. Plant cells can be differentiated or undifferentiated (e.g., callus, suspension culture cells, protoplasts, leaf cells, root cells, phloem cells, and pollen).

The present disclosure encompasses inserting one or more gene constructs comprising one or more nucleic acid sequences, which encode an affinity construct of the disclosure, into the genome of plants, in particular of agricultural crops, or expressing these gene constructs ex planta (e.g., in recombinant bacteria) and applying the purified protein to the plant or the insect pest (e.g., by spraying).

The present disclosure also encompasses inserting one or more gene constructs comprising one or more nucleic acid sequences, which encode a novel affinity construct of the disclosure and an insecticidal protein (wherein the insecticidal protein corresponds to the insecticidal protein (toxin), which the at least one affinity molecule B of the novel affinity construct is binding to, or is binding to, or is being directed to, or is being designed to bind to), into the genome of plants, in particular of agricultural crops, or expressing these gene constructs ex planta (e.g., in recombinant bacteria) and applying the purified proteins to the plant or the insect pest (e.g., by spraying).

When applying the affinity construct to/on the plant, an insect will take up and ingest the affinity construct of the disclosure upon feeding on the plant. In case of applying the novel affinity construct to/on the plant, also an insecticidal protein is applied to/on the plant (wherein the insecticidal protein corresponds to the insecticidal protein (toxin), which the at least one affinity molecule B of the novel affinity construct is binding to, or is being directed to, or is being designed to bind to), so that an insect will take up and ingest both the affinity construct and the insecticidal protein of the disclosure upon feeding on the plant.

When applying the affinity construct and the insecticidal protein directly on the insect pest or the habitat where the insect is living, then the insect pest will take up the affinity construct and the insecticidal protein upon contact. In various aspects of the present disclosure, the gene construct is a vector or a plasmid.

Furthermore, the expression of the affinity construct of the disclosure, and/or an insecticidal protein, in transgenic plants can be achieved using constitutively active promoters (e.g., the 35S promoter or Ubiquitin promoters), or using specific promoters that allow increasing expression of the affinity construct and/or the insecticidal protein in areas that are attacked by the target insects, or that can be induced via external cues (e.g., chemically-inducible promoters, heat-inducible promoters).

Transformation protocols as well as protocols for introducing nucleotide sequences into plants may vary depending on the type of plant or plant cell, i.e., monocot or dicot, targeted for transformation but are widely known in the art. Suitable methods of introducing nucleotide sequences into plant cells and subsequent insertion into the plant genome include, but are not limited to, microinjection, electroporation, Agrobacterium-mediated transformation, direct gene transfer and ballistic particle acceleration. Additional transformation procedures can be found, e.g., in Weissinger, et al. 1988, Ann. Rev. Genet. 22:421-477. In various embodiments, the nucleic acid sequences of the disclosure can be provided to a plant using a variety of transient transformation methods. Such transient transformation methods include, but are not limited to, the introduction of the affinity construct and/or insecticidal protein of the disclosure, or variants and fragments thereof, directly into the plant or the introduction of the affinity construct transcript and/or insecticidal protein transcript, into the plant. Such methods include, for example, microinjection or particle bombardment.

Alternatively, polynucleotides encoding affinity constructs and/or insecticidal proteins of the disclosure, or variants and fragments thereof, can be transiently transformed into the plant using techniques known in the art, including, but not limited to, viral vector systems. The affinity construct and/or insecticidal protein of the present disclosure can be introduced to the plant by means including, but not limited to, viral vectors, bacteria, injection, grafting, spraying and the like.

Plant transformation vectors may comprise of one or more DNA vectors needed for achieving plant transformation. For example, it is a common practice in the art to utilize plant transformation vectors that comprise more than one contiguous DNA segment. These vectors are often referred to in the art as “binary vectors”. Binary vectors as well as vectors with helper plasmids are often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a “gene of interest” (a gene engineered to be capable of expression in a plant cell for which generation of transgenic plants is desired).

In general, plant transformation methods involve transferring heterologous DNA into target plant cells (e.g., immature or mature embryos, suspension cultures, undifferentiated callus, protoplasts, etc.), followed by applying a maximum threshold level of appropriate selection (depending on the selectable marker gene) to recover the transformed plant cells from a group of untransformed cell mass. Following integration of heterologous foreign DNA into plant cells, one then applies a maximum threshold level of appropriate selection in the medium to kill the untransformed cells and separate and proliferate the putatively transformed cells that survive from this selection treatment by transferring regularly to a fresh medium. By continuous passage and challenge with appropriate selection, one identifies and proliferates the cells that are transformed with the plasmid vector. Alternatively, transgenic plants can be produced by the use of marker genes that do not rely on antibiotic or herbicide resistance but instead promote regeneration after transformation. Molecular and biochemical methods can then be used to confirm the presence of the integrated heterologous gene of interest into the genome of the transgenic plant. The transformation of plants can be based on the use of a standard Agrobacterium-mediated transformation protocol (e.g., as described in Hiei and Komari 1997, Plant Mol Biol 35(1-2): 205-18).

The present disclosure also encompasses marker-free plants that are based on strategies (site-specific recombination, homologous recombination, transposition and co-transformation) that have been developed to eliminate the marker gene efficiently from the nuclear or chloroplast genome soon after selection.

The present disclosure further encompasses marker-free transformation of plants and marker-free plants resulting therefrom, preferably marker-free transformation of monocotyledons and marker-free monocotyledons resulting therefrom. The improvement of the transformation efficiency enables the production of transformed plants with without the need to introduce any selection marker, as discussed in EP2274973A1. The efficiency of transformation can be improved to increase the percentage of transformed cells among non-transformed cells, and this state can be maintained until regeneration of whole plants. A marker-free transformation of plants may be performed according to an Agrobacterium-mediated method comprising the steps (a) culturing an Agrobacterium-inoculated plant material with a co-culture medium that provides for an enhanced transformation efficiency; and (b) regenerating the tissue obtained in step (a) with a regeneration medium to thereby regenerate a transgenic plant, wherein the method does not contain a marker gene-based selection step.

The cells that have been transformed may be grown or regenerated into plants in accordance with conventional methods. These plants may then be grown, and either pollinated with the same transformed strain or different strains and the resulting hybrid having constitutive or inducible expression of the desired phenotypic characteristic identified. Two or more generations may be grown to ensure that expression of the desired phenotypic characteristic is stably maintained and inherited.

The nucleotide sequences of the disclosure may be provided to the plant by contacting the plant with a virus or viral nucleic acids. Generally, such methods involve incorporating the nucleotide construct of interest within a viral DNA or RNA molecule. Methods for providing plants with nucleotide constructs and producing the encoded proteins in the plants, which involve viral DNA or RNA molecules, are known in the art.

The disclosure further relates to plant-propagating material of a transformed plant of the disclosure including, but not limited to, seeds, tubers, corms, bulbs, leaves, and cuttings of roots and shoots.

The disclosure may be used for transformation of any plant species, including, but not limited to, monocots and dicots. Examples of plants of interest include, but are not limited to, grain plants that provide seeds of interest, e.g., corn (Zea mays).

The disclosure encompasses a plant or progeny thereof, comprising one or more nucleic acid molecules encoding an affinity construct of the disclosure and/or an insecticidal protein of the disclosure. The disclosure also encompasses a plant or progeny thereof or plant parts, stably transformed with one or more nucleic acid molecules encoding an affinity construct of the disclosure and/or an insecticidal protein. The disclosure encompasses seed or grain of the plant or progeny thereof of the disclosure, wherein the seed or grain comprises one or more nucleic acid molecules encoding an affinity construct of the disclosure and/or an insecticidal protein. The disclosure also encompasses a biological sample from a tissue or seed of a plant or progeny thereof of the disclosure. In preferred embodiments, the plant is a monocotyledonous plant. In various other preferred embodiments, the plant is a dicotyledonous plant. In various embodiments, the plant is any of barley, corn, oat, rice, rye, sorghum, turf grass, sugarcane, wheat, alfalfa, banana, broccoli, bean, cabbage, canola, carrot, cassava, cauliflower, celery, citrus, cotton, a cucurbit, eucalyptus, flax, garlic, grape, onion, lettuce, pea, peanut, pepper, potato, poplar, pine, sunflower, safflower, soybean, strawberry, sugar beet, sweet potato, tobacco, tomato ornamental, shrub, nut, chickpea, pigeon pea, millets, hops, and pasture grasses. More specifically, a plant according to the present disclosure may be any one of barley (Hordeum vulgare), sorghum (Sorghum bicolor), rye (Secale cereale), Triticale, sugar cane (Saccharum officinarum), maize (Zea mays), foxtail millet (Setaria italic), rice (Oryza sativa), Oryza minuta, Oryza australiensis, Oryza alta, wheat (Triticum aestivum), Triticum durum, Hordeum bulbosum, purple false brome (Brachypodium distachyon), sea barley (Hordeum marinum), goat grass (Aegilops tauschii), apple (Malus domestica), strawberry, sugar beet (Beta vulgaris), sunflower (Helianthus annuus), Australian carrot (Daucus glochidiatus), American wild carrot (Daucus pusillus), Daucus muricatus, carrot (Daucus carota), eucalyptus (Eucalyptus grandis), Erythranthe guttata, Genlisea aurea, woodland tobacco (Nicotiana sylvestris), tobacco (Nicotiana tabacum), Nicotiana tomentosiformis, tomato (Solanum lycopersicum), potato (Solanum tuberosum), coffee (Coffea canephora), grape vine (Vitis vinifera), cucumber (Cucumis sativus), mulberry (Morus notabilis), thale cress (Arabidopsis thaliana), Arabidopsis lyrata, sand rock-cress (Arabidopsis arenosa), Crucihimalaya himalaica, Crucihimalaya wallichii, wavy bittercress (Cardamine flexuosa), peppergrass (Lepidium virginicum), sheperd's-purse (Capsella bursa-pastoris), Olmarabidopsis pumila, hairy rockcress (Arabis hirsuta), rape (Brassica napus), broccoli (Brassica oleracea), Brassica rapa, Brassica juncacea, black mustard (Brassica nigra), radish (Raphanus sativus), Eruca vesicaria sativa, orange (Citrus sinensis), Jatropha curcas, cotton (Gossipium sp.), soybean (Glycine max), and black cottonwood (Populus trichocarpa). Preferably, the plant is any one of barley (Hordeum vulgare), rye (Secale cereale), Triticale, maize (Zea mays), rice (Oryza sativa), wheat (Triticum aestivum), and soybean (Glycine max).

In various embodiments, the plant comprises one or more additional transgenic or non-transgenic traits. In various embodiments, the one or more additional transgenic or non-transgenic traits is any of insect resistance, herbicide resistance, fungal resistance, virus resistance or stress tolerance, disease resistance, male sterility, stalk strength, increased yield, modified starches, improved oil profile, balanced amino acids, high lysine or methionine, increased digestibility, improved fiber quality, drought resistance or tolerance, cold resistance or tolerance, salt resistance or tolerance, and increased yield under stress. In various other embodiments, the one or more additional transgenic or non-transgenic traits is any of moisture at harvest, increased sugar content, flowering control, increased biomass, altered secondary plant metabolites, and altered plant-plant interaction abilities (increased crop densities). Non-transgenic traits can be “stacked” in the plant of the present disclosure comprising the insecticidal fusion protein of the disclosure by breeding (so-called breeding stacks). Transgenic traits can be “stacked” in the plant of the present disclosure comprising the insecticidal fusion protein of the disclosure either by molecular means (transformation by more than one genetic constructs or by subsequent transformation (so-called molecular stacks) or breeding (so-called breeding stacks). Both breeding and molecular stacking are described below.

The disclosure also encompasses a plant comprising an expression cassette of the present disclosure. The disclosure also encompasses a plant cell or a plant part or a plant seed comprising an expression cassette of the present disclosure. The disclosure further encompasses a microbial cell comprising an expression cassette of the present disclosure.

The present disclosure encompasses a plant, plant part or plant seed capable of expressing an affinity construct of the disclosure and/or an insecticidal protein. Accordingly, the disclosure also encompasses a method for expressing in a plant, plant part or plant seed an affinity construct of the disclosure and/or an insecticidal protein, comprising the steps of: (a) inserting into a plant cell a nucleic acid sequence comprising in 5′ to 3′ direction an operably linked recombinant, double-stranded DNA molecule, wherein the recombinant double-stranded DNA molecule comprises (i) a promoter that functions in the plant cell; (ii) one or more nucleic acid molecules encoding an affinity construct of the disclosure and/or an insecticidal protein; and (iii) a 3′ non-translated polynucleotide that functions in the cells of the plant to cause termination of transcription; (b) obtaining a transformed plant cell comprising the nucleic acid sequence of step (a); and (c) generating from the transformed plant cell a plant, plant part or plant seed capable of expressing an affinity construct of the disclosure and/or an insecticidal protein. In other embodiments, methods are encompassed wherein in a plant, plant part or plant seed more than one affinity construct of the disclosure and/or more than one insecticidal protein is expressed. The disclosure encompasses a plant, plant part or plant seed produced by such methods. Such a plant, plant part or plant seed may comprise one or more additional transgenic or non-transgenic trait. In various embodiments, the one or more additional transgenic or non-transgenic trait is any one of the one or more additional transgenic or non-transgenic traits mentioned above.

### Transformation of Microbes

The novel affinity construct of the present disclosure can be easily co-expressed with an insecticidal protein in transgenic microbial cells, or can be applied, in combination with an insecticidal protein, as an insecticidal spray/solution to a plant, seed or insect, in particular in agricultural crops or cells thereof, wherein the insecticidal protein (toxin) corresponds to the insecticidal protein (toxin) against which the at least one affinity molecule B of the novel affinity construct is capable of binding to, or is binding to, or is being directed to, or is being designed to bind to. Furthermore, the affinity molecules, single domain antibodies can be readily expressed by transformed plants or microbial cells.

The present disclosure provides a microbial cell comprising (i) one or more nucleic acid sequences encoding a novel affinity construct of the present disclosure and/or an insecticidal protein, wherein the insecticidal protein corresponds to the insecticidal protein which the at least one affinity molecule B of the novel affinity construct is capable of binding to, or is binding to, or is being directed to, or is being designed to bind to, or (i) one or more vectors comprising one or more nucleic acid sequences encoding a novel affinity construct of the present disclosure and/or an insecticidal protein, wherein the insecticidal protein corresponds to the insecticidal protein which the at least one affinity molecule B of the novel affinity construct is capable of binding to, or is binding to, or is being directed to, or is being designed to bind to.

The present disclosure also provides a microbial cell comprising a nucleic acid molecule encoding an insecticidal affinity construct of the present disclosure.

In other embodiments, microbial cells are encompassed wherein in these microbial cells more than one affinity construct of the disclosure and/or more than one insecticidal protein is expressed.

The methods of the present disclosure involve introducing a novel affinity construct of the disclosure or a polynucleotide encoding same, and/or an insecticidal protein or a polynucleotide encoding same, into a microbial cell. “Introducing” is intended to mean presenting to a microbial cell the novel affinity construct of the disclosure or a polynucleotide encoding same, and/or an insecticidal protein or a polynucleotide encoding same in such a manner that the sequence gains access to the interior of a microbial cell. The methods of the disclosure do not depend on a particular method for introducing a polynucleotide or polypeptide into a microbial cell, what is relevant is that the polynucleotide or polypeptides gains access to the interior of at least one microbial cell. Methods for introducing polynucleotide or polypeptides into microbial cells are well known in the art including, but not limited to, stable transformation methods, transient transformation methods and virus-mediated methods. The terms “stable transformation”, “transient transformation” methods and “virus-mediated transformation” have essentially the same meaning as outline above.

The present disclosure also encompasses inserting one or more gene constructs comprising one or more nucleic acid sequences, which encode a novel affinity construct of the disclosure and/or an insecticidal protein (wherein the insecticidal protein corresponds to the insecticidal protein which the at least one affinity molecule B of the novel affinity construct is capable of binding to, or is binding to, or is being directed to, or is being designed to bind to), into the genome of microbial cells, or expressing these gene constructs ex planta in the recombinant microbial cell and applying the purified proteins or the microbial cells to the plant or the insect pest (e.g., by spraying).

When applying the affinity construct in combination with an insecticidal protein corresponding to the insecticidal protein, which the at least one affinity molecule B of the novel affinity construct is capable of binding to, or is binding to, or is being directed to, or is being designed to bind to or when applying the recombinant microbial cells to/on the plant, an insect will take up and ingest the affinity construct of the disclosure or the recombinant microbial cell upon feeding on the plant. When applying the affinity construct and the corresponding insecticidal protein (toxin) or the recombinant microbial cell directly on the insect pest or the habitat where the insect is living, then the insect pest will take up the affinity construct and the insecticidal protein (toxin) or the recombinant microbial cell of the disclosure upon contact. In various aspects of the present disclosure, the gene construct is a vector or a plasmid.

Furthermore, the expression of the affinity construct of the disclosure and/or of the insecticidal protein (toxin) in transgenic microbial cells can be achieved using constitutively active promoters or using specific promoters that can be induced via external cues (e.g., chemically-inducible promoters, heat-inducible promoters).

Transformation protocols as well as protocols for introducing nucleotide sequences into microbial cells may vary depending on the type of microbial cell, targeted for transformation but are widely known in the art. In various embodiments, the nucleic acid sequences of the disclosure can be provided to a microbial cell using a variety of transient transformation methods. Such methods are known in the art.

The disclosure encompasses a recombinant microbial cell or progeny thereof, comprising a nucleic acid molecule encoding an affinity construct of the disclosure and/or an insecticidal protein, wherein the insecticidal protein corresponds to the insecticidal protein which the at least one affinity molecule B of the novel affinity construct is binding to or directed to. The disclosure also encompasses recombinant microbial cells or progeny thereof stably transformed with a nucleic acid molecule encoding an affinity construct of the disclosure and/or an insecticidal protein, wherein the insecticidal protein corresponds to the insecticidal protein which the at least one affinity molecule B of the novel affinity construct is capable of binding to, or is binding to, or is being directed to, or is being designed to bind to.

The disclosure also encompasses a recombinant microbial cell comprising an expression cassette of the present disclosure.

The present disclosure encompasses a recombinant microbial cell capable of expressing an affinity construct of the disclosure and/or an insecticidal protein, wherein the insecticidal protein corresponds to the insecticidal protein which the at least one affinity molecule B of the novel affinity construct is binding to or directed to. Accordingly, the disclosure also encompasses a method for expressing in a microbial cell an affinity construct of the disclosure and/or an insecticidal protein, wherein the insecticidal protein corresponds to the insecticidal protein which the at least one affinity molecule B of the novel affinity construct is capable of binding to, or is binding to, or is being directed to, or is being designed to bind to, comprising the steps of: (a) inserting into a microbial cell a nucleic acid sequence comprising in 5′ to 3′ direction an operably linked recombinant, double-stranded DNA molecule, wherein the recombinant double-stranded DNA molecule comprises (i) a promoter that functions in the microbial cell; (ii) a nucleic acid molecule encoding an affinity construct of the disclosure and/or an insecticidal protein, wherein the insecticidal protein corresponds to the insecticidal protein which the at least one affinity molecule B of the novel affinity construct is capable of binding to, or is binding to, or is being directed to, or is being designed to bind to; and (iii) a 3′ non-translated polynucleotide that functions in the microbial cell to cause termination of transcription; and (b) obtaining a transformed microbial cell comprising the nucleic acid sequence of step (a) and capable of expressing an affinity construct of the disclosure and/or an insecticidal protein, wherein the insecticidal protein corresponds to the insecticidal protein which the at least one affinity molecule B of the novel affinity construct is capable of binding to, or is binding to, or is being directed to, or is being designed to bind to. The disclosure encompasses a microbial cell produced by such a method.

### Evaluation of Plant Transformation

Following introduction of heterologous foreign DNA into plant cells, the transformation or integration of heterologous gene in the plant genome is confirmed by various methods such as analysis of nucleic acids, proteins and metabolites associated with the integrated gene. For example, plant transformation may be confirmed by Southern blot analysis of genomic DNA. PCR analysis is also a rapid method to screen transformed cells, tissue or shoots for the presence of incorporated gene at the earlier stage before transplanting into the soil (Sambrook and Russell, (2001) Molecular Cloning: A Laboratory Manual. Cold Spring Harbor Laboratory Press, Cold Spring Harbor, NY).

The above-described methods for confirming transformation or integration of the heterologous gene(s) in the plant genome can also be applied to microbial cells. The person skilled in the art is aware of respective methods that can be applied to microbial cells.

### Stacking of Traits in Transgenic Plant

The present disclosure also encompasses the stacking of multiple affinity constructs of the present disclosure. The stacked affinity constructs of the present disclosure are either directed against the same insect-specific structures, preferably receptors, in or on the same insect pest, are directed against different insect-specific structures, preferably receptors, in or on the same insect pest, or are directed against different insect-specific structures, preferably receptors, in or on different insect pests. The present disclosure also encompasses the stacking of multiple affinity constructs of the present disclosure, wherein the stacked affinity constructs of the present disclosure are either directed against the same insecticidal protein (toxin) or are directed against different insecticidal proteins (toxins). If the stacked insecticidal proteins are directed against the same pest, then there are the following options: (1) the one or more affinity molecule A is the same (stacking would then lead to higher concentrations of the same insecticidal protein), (2) the one or more affinity molecule A is the same but the insecticidal proteins are different while still attacking the same pest (to use, e.g., an abundant receptor to address the pest with different insecticidal mode of actions), (3) the one or more affinity molecule A is different but the insecticidal proteins are the same (to address different receptors with one (insecticidal) mode of action). If the stacked insecticidal proteins are directed against different pests, then there are the following options: (1) the one or more affinity molecule A and insecticidal proteins are the same (stacking would lead to higher concentrations of the same insecticidal protein), (2) the one or more affinity molecule A are the same but the insecticidal proteins are different in order to attack different insect pests (to use, e.g., an abundant receptor to address the pest with different (insecticidal) mode of actions), (3) the one or more affinity molecule A is different but the insecticidal proteins are the same (to address different receptors with one working insecticidal mode of action that affects different insects). Such approaches are also useful to address the probability of resistance to insecticidal proteins.

Transgenic plants may also comprise a stack (or pyramid) of one or more polynucleotides encoding an affinity construct disclosed herein with one or more additional polynucleotides encoding different traits resulting in the production or suppression of multiple polypeptide sequences. Transgenic plants comprising stacks of polynucleotide sequences can be obtained by either traditional breeding methods or through genetic engineering methods or both. These methods include, but are not limited to, crossing individual lines each comprising one or more polynucleotides of interest, transforming a transgenic plant comprising one or more transgenes disclosed herein with a subsequent transgene, and co-transformation of transgenes into a single plant cell. As used herein, the term “stacked” includes having two or more traits present in the same plant (e.g., both traits are incorporated into the nuclear genome, or one trait is incorporated into the nuclear genome and one trait is incorporated into the genome of a plastid, or both traits are incorporated into the genome of a plastid). In one non-limiting example, “stacked traits” comprise a molecular stack where the sequences are physically adjacent to each other. A trait, as used herein, refers to the phenotype derived from a particular sequence or groups of sequences. Co-transformation of genes can be carried out using single transformation vectors comprising multiple genes or genes carried separately on multiple vectors. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. Expression of the sequences can be driven by the same promoter or by different promoters. It is further recognized that polynucleotide sequences can be stacked at a desired genomic location using a site-specific recombination system.

In various embodiments the polynucleotides encoding an affinity construct of the disclosure, alone or stacked with one or more additional insect resistance traits can be stacked with one or more additional transgenic or non-transgenic input traits (e.g., herbicide resistance, fungal resistance, virus resistance or stress tolerance, disease resistance, male sterility, stalk strength, and the like) or transgenic or non-transgenic output traits (e.g., increased yield, modified starches, improved oil profile, balanced amino acids, high lysine or methionine, increased digestibility, improved fiber quality, drought resistance, and the like). Thus, the polynucleotide embodiments can be used to provide a complete agronomic package of improved crop quality with the ability to flexibly and cost effectively control any number of agronomic (insect) pests.

In some embodiments the affinity constructs of the disclosure are useful as part of an insect resistance management strategy in combination (i.e., pyramided or stacked) with other pesticidal or insecticidal proteins or topical application of one or more insecticide to the plant. Provided are methods of controlling Orthopteran, Thysanopteran, Hymenopteran, Dipteran, Lepidopteran, Coleopteran and/or Hemipteran insect infestation(s) in a transgenic plant that promote insect resistance management, comprising expressing in the plant at least two different insecticidal proteins having different modes of action, one of them being part of the insecticidal fusion protein of the disclosure.

Transgenes useful for stacking include but are not limited to: (i) transgenes that confer resistance to insects or disease, (ii) transgenes that confer resistance to a herbicide, (iii) transgenes that confer or contribute to an altered grain characteristic, (iv) genes that control male-sterility, (v) genes that create a site for site specific DNA integration, (vi) genes that affect abiotic stress resistance, (vii) genes that confer increased yield, and (viii) genes that confer plant digestibility.

Use in Insect Pest Control

General methods for employing insecticidal proteins or nucleic acid sequences encoding same in pesticide control or in genetic engineering of organisms that are used as pesticidal agents are known in the art.

Microorganism hosts that are known to occupy the “phytosphere” (phylloplane, phyllosphere, rhizosphere, and/or rhizoplana) of one or more crops of interest may be selected. These microorganisms provide for stable maintenance and expression of the gene expressing an affinity construct of the disclosure, and desirably, provide for improved protection of the affinity construct from environmental degradation and inactivation.

Nucleic acid sequences encoding a novel affinity construct of the disclosure and an insecticidal protein (wherein the insecticidal protein corresponds to the insecticidal protein which the at least one affinity molecule B of the novel affinity construct is capable of binding to, or is binding to, or is being directed to, or is being designed to bind to) can be introduced into a wide variety of microbial hosts. Expression of nucleic acid sequences encoding the novel affinity construct and the insecticidal protein results, directly or indirectly, in the intracellular production and maintenance of the affinity construct and the insecticidal protein. With suitable hosts, e.g., Pseudomonas, the microbes can be applied to a place where they will proliferate and be ingested by the insects. The result is a control of the insect pest.

Suitable microorganisms include bacteria, algae, and fungi. Of particular interest are phytosphere bacterial species such as, e.g., Pseudomonas fluorescens, Agrobacteria, Rhizobia etc. Of particular interest are also root-colonizing bacteria. Nucleic acid sequences encoding affinity constructs of the disclosure and/or insecticidal proteins can be introduced, for example, into the root-colonizing Bacillus by means of electro transformation. Furthermore, expression systems can be designed so that affinity constructs of the disclosure and/or insecticidal proteins are secreted outside the cytoplasm of gram-negative bacteria, such as, e.g., E. coli.

The novel affinity constructs of the disclosure can be fermented in a bacterial host and the resulting bacteria processed, formulated together with an insecticidal protein, and then used as a microbial spray in the same manner that Bacillus thuringiensis strains have been used as insecticidal sprays. Any suitable microorganism can be used for this purpose. Methods of transforming microbial hosts, fermenting same and of collecting, isolating or extracting recombinant proteins from the culture medium or the cultured microbial hosts are well known in the art and also addressed elsewhere herein.

In various embodiments of the methods of controlling insect infestation in a transgenic plant and promoting insect resistance management, the composition of the present disclosure comprising the novel affinity construct and an insecticidal protein (toxin) comprises one or more insecticidal protein(s) insecticidal to insects in the Orthopteran, Thysanopteran, Hemipteran, Hymenopteran, Dipteran, Lepidopteran and/or Coleopteran order of insects. Also provided are means for effective insect resistance management of transgenic plants, comprising co-expressing at high levels in the plants two or more insecticidal proteins toxic to insects, in particular toxic to Orthopteran, Thysanopteran, Hemipteran, Hymenopteran, Dipteran, Lepidopteran and/or Coleopteran insects, but each insecticidal protein exhibiting a different mode of effectuating its inhibiting growth or killing activity.

The disclosure encompasses a method for controlling an insect pest population, comprising contacting the insect pest population with an insecticidally-effective amount of a composition comprising a novel affinity construct of the disclosure and an insecticidal protein, wherein the insecticidal protein corresponds to the insecticidal protein which the at least one affinity molecule B of the affinity construct of the disclosure is capable of binding to, or is binding to, or is being directed to, or is being designed to bind to.

The disclosure further encompasses a method of inhibiting growth or killing an insect pest, comprising contacting the insect pest with an insecticidally-effective amount of a composition comprising a novel affinity construct of the disclosure and an insecticidal protein, wherein the insecticidal protein corresponds to the insecticidal protein which the at least one affinity molecule B of the affinity construct of the disclosure is capable of binding to, or is binding to, or is being directed to, or is being designed to bind to.

The disclosure still further encompasses a method for controlling an insect pest population resistant to a pesticidal protein, comprising contacting the resistant insect pest population with an insecticidally-effective amount of a composition comprising an affinity construct of the disclosure and an insecticidal protein, wherein the insecticidal protein corresponds to the insecticidal protein which the at least one affinity molecule B of the affinity construct of the disclosure is capable of binding to, or is binding to, or is being directed to, or is being designed to bind to is (capable of) binding to or is directed to.

The disclosure further encompasses a method for protecting a plant from an insect pest, comprising expressing in the plant or cell thereof an affinity construct of the disclosure and an insecticidal protein, wherein the insecticidal protein corresponds to the insecticidal protein which the at least one affinity molecule B of the affinity construct of the disclosure is capable of binding to, or is binding to, or is being directed to, or is being designed to bind to.

The disclosure still further encompasses a method for controlling an insect infestation in a transgenic plant and/or providing insect resistance management, comprising expressing in the plant (i) an affinity construct of the disclosure, (ii) a first insecticidal protein, wherein the first insecticidal protein corresponds to the insecticidal protein which the at least one affinity molecule B of the affinity construct of the disclosure is capable of binding to, or is binding to, or is being directed to, or is being designed to bind to, and (iii) at least one additional insecticidal protein, wherein the at least one additional insecticidal protein and the first insecticidal protein have different modes of action. In various embodiments, the insect infestation is an Orthopteran, Thysanopteran, Hemipteran, Hymenopteran, Dipteran, Lepidopteran and/or Coleopteran insect infestation.

The present disclosure provides a method for protecting a plant against an insect pest, comprising the steps: (i) transforming a plant with one or more nucleic acid sequences encoding a novel affinity construct of the present disclosure and an insecticidal protein, wherein the insecticidal protein corresponds to the insecticidal protein, which the at least one affinity molecule B of the affinity construct of the disclosure is capable of binding to, or is binding to, or is being directed to, or is being designed to bind to, and (ii) expressing the affinity construct and the insecticidal protein in the plant. The plant may be a monocotyledonous or a dicotyledonous plant.

The present disclosure further provides a method for increasing the binding efficiency of an insecticidal protein to its receptor, comprising the steps: (i) transforming a plant with one or more nucleic acid sequences encoding a novel affinity construct of the present disclosure and an insecticidal protein, wherein the insecticidal protein corresponds to the insecticidal protein, which the at least one affinity molecule B of the affinity construct of the disclosure is capable of binding to, or is binding to, or is being directed to, or is being designed to bind to; and (ii) expressing the affinity construct and the insecticidal protein in the plant. The plant may be a monocotyledonous or a dicotyledonous plant.

The present disclosure further provides a method for preparing a plant exhibiting resistance against an insect pest, comprising the steps: (i) transforming a plant with one or more nucleic acid sequences encoding a novel affinity construct of the present disclosure and an insecticidal protein, wherein the insecticidal protein corresponds to the insecticidal protein, which the at least one affinity molecule B of the affinity construct of the disclosure is capable of binding to, or is binding to, or is being directed to, or is being designed to bind to; and (ii) expressing the affinity construct and the insecticidal protein in the plant. The plant may be a monocotyledonous or a dicotyledonous plant.

Still further, the present disclosure provides a method for protecting a plant against an insect pest, comprising applying to said plant an insecticidal composition comprising a novel affinity construct of the disclosure and an insecticidal protein, wherein the insecticidal protein corresponds to the insecticidal protein, which the at least one affinity molecule B of the affinity construct of the disclosure is capable of binding to, or is binding to, or is being directed to, or is being designed to bind to. The plant may be a monocotyledonous or a dicotyledonous plant.

Still further, the present disclosure provides the use of (i) an affinity construct of the present disclosure in combination with an insecticidal protein, wherein the insecticidal protein corresponds to the insecticidal protein, which the at least one affinity molecule B of the novel affinity construct is capable of binding to, or is binding to, or is being directed to, or is being designed to bind to, or (ii) one or more nucleic acid sequences encoding a novel affinity construct of the present disclosure and an insecticidal protein, wherein the insecticidal protein corresponds to the insecticidal protein, which the at least one affinity molecule B of the novel affinity construct is capable of binding to, or is binding to, or is being directed to, or is being designed to bind to, or (iii) a vector comprising the said one or more nucleic acid sequences, or (iv) a composition comprising a novel of the present disclosure and an insecticidal protein, wherein the insecticidal protein corresponds to the insecticidal protein, which the at least one affinity molecule B of the novel affinity construct is capable of binding to, or is binding to, or is being directed to, or is being designed to bind to, for protecting a plant against an insect pest. The plant may be a monocotyledonous or a dicotyledonous plant.

In any of the above methods and uses, the plant may preferably be modified by using known genome editing tools for delivery of constructs, either through Agrobacterium-mediated transfer, electroporation, micro-projectile bombardment, virus-mediated delivery, or sexual cross. The techniques of Agrobacterium-mediated transfer, electroporation, micro-projectile bombardment, virus-mediated delivery and sexual cross are well-known to the skilled person and methods are described in the literature. The same holds for genome editing tools like, e.g., TAL Effector Nucleases (TALEN), CRISPR/Cas9, and CRSPR/cpf1, etc.

### Bioassay for Insecticidal Toxins

The final formulation of a composition comprising the novel affinity construct and an insecticidal protein (toxin) of the disclosure can be bioassayed against an accepted international standard using a specific test insect (see, for example, e.g., Baum et al. 2004, (Appl. Environ. Microb., 4889-4898). The standardization allows comparison of different formulations in the laboratory.

Methods for measuring insecticidal activity are well known in the art. See, for example, Dhadialla & Gill 2014, Advances in Insect Physiology, Edition 47, “Insect Midgut and Insecticidal Proteins” Academic Press. Generally, the protein is mixed and used in feeding assays. Such assays can include contacting plants with one or more pests and determining the plant's ability to survive and/or cause the death of the pests.

### Methods for Increasing Plant Yield

Methods for increasing plant yield are provided. The methods comprise providing a plant or plant cell expressing one or more polynucleotides encoding a novel affinity construct of the disclosure and/or an insecticidal protein, wherein the insecticidal protein corresponds to the insecticidal protein, which the at least one affinity molecule B of the novel affinity construct is capable of binding to, or is binding to, or is being directed to, or is being designed to bind to, and growing the plant or a seed thereof in a field infested with an insect pest against which the affinity construct in combination with the insecticidal protein has insecticidal activity. In various embodiments, the the affinity construct in combination with the insecticidal protein has insecticidal activity against an Isopteran, Blattodean, Orthopteran, Phthirapteran, Thysanopteran, Hemipteran, Hymenopteran, Siphonapteran, Dipteran, Coleopteran and/or Lepidopteran or nematode pest, and the field is infested with an Isopteran, Blattodean, Orthopteran, Phthirapteran, Thysanopteran, Hemipteran, Hymenopteran, Siphonapteran, Dipteran, Coleopteran, Lepidopteran and/or nematode pest, respectively. As defined herein, the “yield” of the plant refers to the quality and/or quantity of biomass produced by the plant. By “biomass” is intended any measured plant product, including, but not limited to, plant organs that are specifically harvested, e.g., leaves, grain, roots, seeds, stalks, flowers, fruits. An increase in biomass production is any improvement in the yield of the measured plant product. Increasing plant yield has several commercial applications. For example, increasing plant leaf biomass may increase the yield of leafy vegetables for human or animal consumption. Additionally, increasing leaf biomass can be used to increase production of plant-derived pharmaceutical or industrial products, which in case of maize includes, without being limited thereto, food/feedstock, biogas and biofuel. An increase in yield can comprise any statistically significant increase including, but not limited to, at least a 1% increase, at least a 3% increase, at least a 5% increase, at least a 10% increase, at least a 20% increase, at least a 30%, at least a 50%, at least a 70%, at least a 100% or a greater increase in yield as compared to the yield that is obtained from a plant not expressing the insecticidal sequences encoding a novel affinity construct of the disclosure and/or an insecticidal protein (wherein the insecticidal protein corresponds to the insecticidal protein, which the at least one affinity molecule B of the novel affinity construct is capable of binding to, or is binding to, or is being directed to, or is being designed to bind to. In specific methods, plant yield is increased as a result of improved pest resistance of a plant expressing a novel affinity construct of the disclosure and/or an insecticidal protein (wherein the insecticidal protein corresponds to the insecticidal protein, which the at least one affinity molecule B of the novel affinity construct is capable of binding to, or is binding to, or is being directed to, or is being designed to bind to. Expression of the novel affinity construct of the disclosure and/or an insecticidal protein (wherein the insecticidal protein corresponds to the insecticidal protein, which the at least one affinity molecule B of the novel affinity construct is capable of binding to, or is binding to, or is being directed to, or is being designed to bind to results in a reduced ability of an insect pest to infest or feed on the plant, thus improving plant yield.

### Compositions

The present disclosure encompasses an (insecticidal) composition comprising an affinity construct of the disclosure which in turn comprises at least one affinity molecule A and at least one affinity molecule B. Preferably, the composition is formulated as a spray.

The present disclosure further encompasses an insecticidal composition comprising an insecticidally-effective amount of the combination of an affinity construct of the disclosure and an insecticidal protein, wherein the insecticidal protein corresponds to the insecticidal protein which the at least one of affinity molecule B of the affinity construct of the disclosure is capable of binding to, or is binding to, or is being directed to, or is being designed to bind to.

To address the resistance of insect pests to insecticidal proteins caused by mutations in the receptor proteins the composition may further comprise wild type receptor protein(s) from the gut of the target insect at least one of the at least two affinity molecules A present in the composition is designed to recognize. “Wild type” with regards to the receptor protein means that the receptor protein is in its susceptible form, i.e., without the one or more mutations that in resistant insect pests confer resistance against certain insecticidal proteins. After uptake by the insect pest these wild type receptor proteins insert themselves into the insect gut either in addition to the mutated receptor proteins or by replacing them. Either way, the presence of wild type receptor proteins allows the insecticidal protein to bind, to insert into the membrane, to form a pore and eventually to kill the insect.

The composition may furthermore comprise an agriculturally suitable or agriculturally acceptable component. Examples of such components include water, plant oils, essential oils, emulsifiers, thickeners, suspension agents, dispersion agents, anti-freeze agents, adjuvants, carriers or excipients, and wetting agents. Suitable plant oils for inclusion in the compositions of the present disclosure include canola oil (oilseed rape oil), soybean oil, cottonseed, castor oil, linseed oil and palm oil. Suitable emulsifiers for use in the compositions of the present disclosure include any known agriculturally acceptable emulsifier. In particular, the emulsifier may comprise a surfactant such as: alkylaryl sulphonates, ethoxylated alcohols, polyalkoxylated butyl ethers, calcium alkyl benzene sulphonates, polyalkylene glycol ethers and butyl polyalkylene oxide block copolymers as are known in the art. Nonyl phenol emulsifiers such as Triton N57™ are particular examples of emulsifiers, which may be used in the compositions of the disclosure, as are polyoxyethylene sorbitan esters such as polyoxyethylene sorbitan monolaurate (sold by ICI under the trade name “Tween™”). In some instances, natural organic emulsifiers may be preferred, particularly for organic farming applications. Coconut oils such as coconut diethanolamide is an example of such an compound. Palm oil products such as lauryl stearate may also be used. Examples of thickeners which may be present in the compositions of the present disclosure comprise gums, for example xanthan gum, or lignosulphonate complexes, as are known in the art. Suitable suspension agents that may be included in the compositions of the present disclosure include hydrophilic colloids (such as polysaccharides, polyvinylpyrrolidone or sodium carboxymethylcellulose) and swelling clays (such as bentonite or attapulgite). Suitable wetting agents for use in the compositions of the present disclosure include surfactants of the cationic, anionic, amphoteric or non-ionic type, as is known in the art. The carrier may be any one of a powder, a dust, pellets, granules, spray, emulsion, colloid, and solution. Preferably, the carrier is a spray. Adjuvants that may be used in compositions of the disclosure include antifoam agents, compatibilizing agents, sequestering agents, neutralizing agents and buffers, corrosion inhibitors, spreading agents, sticking agents, dispersing agents, thickening agents, freeze point depressants, antimicrobial agents, and the like. In various embodiments, the composition further comprises one or more herbicides, insecticides, or fungicides.

The disclosure encompasses the application of an affinity construct of the disclosure in combination with an insecticidal protein, wherein the insecticidal protein corresponds to the insecticidal protein, which the at least one affinity molecule B of the affinity construct of the disclosure is capable of binding to, or binding to, or being directed to, or being designed to bind to, in the form of compositions. The disclosure also encompasses the application of the insecticidal proteins of the disclosure in the form of compositions. Such compositions can be applied to the crop area or plant to be treated simultaneously or in succession with other compounds, such as, e.g., adjuvants, cryoprotectants, surfactants, detergents, pesticidal soaps, selective herbicides, etc. Such compositions may also be time-release or biodegradable carrier formulations that permit long-term dosing of a target area following a single application of the formulation.

Methods of applying an agrochemical composition that contains at least one affinity construct of the disclosure include application to plant parts above the ground, as well as seed coating and soil application. In some embodiments, the at least one affinity construct of the disclosure is applied in combination with one or more insecticidal protein, wherein the one or more insecticidal protein corresponds to the insecticidal protein, which the at least one affinity molecule B of the affinity construct of the disclosure is capable of binding to, or binding to, or being directed to, or being designed to bind to. The number of applications and the rate of application depend on the intensity of infestation by the corresponding insecticidal pest. The composition can be used as insecticidal spray, solution or coating or as further routine application, which are familiar to the skilled person for application of compounds to a plant, plant part (tissue) or plant seed. In a further application, the composition in accordance with the invention is used as a pre-treatment for seed. In this regard, the composition is initially mixed with a carrier substrate and applied to the seeds.

The insecticidal composition may be formulated as a powder, dust, pellet, granule, spray, emulsion, colloid, solution or such like, and may be prepared, if desired, together with further agriculturally acceptable carriers, surfactants or application-promoting adjuvants customarily employed in the art of formulation. Suitable carriers and adjuvants can be solid or liquid and correspond to the substances ordinarily employed in formulation technology, e.g. natural or regenerated mineral substances, solvents, dispersants, wetting agents, binders or fertilizers. Likewise, the formulations may be prepared into edible “baits” or fashioned into pest “traps” to permit feeding or ingestion of the insecticidal formulation by a target pest.

The insecticidal pest ingests or is contacted with, an insecticidally-effective amount of the insecticidal formulation of the disclosure. By “insecticidally-effective amount” is intended an amount of the insecticidal formulation comprising an affinity construct in combination with an insecticidal protein, which the at least one affinity molecule B of the affinity construct of the disclosure is capable of binding to, or is binding to, or is being directed to, or is being designed to bind to, that is able to kill at least one insecticidal pest or to noticeably reduce pest growth (i.e., cause stunting), feeding or normal physiological development. This amount will vary depending on such factors as, for example, the specific target pests to be controlled, the specific environment, location, plant, crop or agricultural site to be treated, the environmental conditions, and the method, rate, concentration, stability, and quantity of application of the insecticidally-effective polypeptide composition. The formulations may also vary with respect to climatic conditions, environmental considerations, and/or frequency of application and/or severity of pest infestation.

The insecticidal formulation comprising an affinity construct of the disclosure which in turn comprises at least one affinity molecule A and at least one affinity molecule B may be a (standard) commercial formulation containing one or more insecticidal proteins and/or microbes for application on plants, plant parts or plant seeds or on the site where the plant to be protected is growing or sown. Such formulations are either containing the one or more insecticidal protein in purified form or are containing one or more microbes that produce the insecticidal protein (either naturally or via transgenesis). Typically, such formulations are formulations containing Bt protein(s). They may, however, also contain other insecticidal toxins, like, for example, proteins or peptides from spider, scorpions or the like.

### Insecticidal Activity

As used herein, insect pests include insects selected from the orders Isoptera, Blattodea, Orthoptera, Phthiraptera, Thysanoptera, Hemiptera, Hymenoptera, Siphonaptera, Diptera, Coleoptera, Lepidoptera, etc., particularly from the orders Lepidoptera and Coleoptera.

The compositions comprising the affinity construct of the disclosure and an insecticidal protein (toxin), wherein the insecticidal protein (toxin) corresponds to the insecticidal protein (toxin) which the at least one affinity molecule B is capable of binding to, or is binding to, or is being directed to, or is being designed to bind to, display entomotoxic activity against insect pests, which may include economically important agronomic, forest, greenhouse, nursery ornamentals, food and fiber, public and animal health, domestic and commercial structure, household and stored product pests.

In various embodiments, the compositions comprising the affinity construct of the disclosure and an insecticidal protein (toxin) of the present disclosure, exhibit insecticidal activity against insect larvae. Of interest are larvae of any of the orders Isoptera, Blattodea, Orthoptera, Phthiraptera, Thysanoptera, Hemiptera, Hymenoptera, Siphonaptera, Diptera, Coleoptera, Lepidoptera, etc., particularly from the orders Lepidoptera and Coleoptera.

### Methods for Inhibiting Growth or Killing an Insect Pest and Controlling an Insect Population

The present disclosure encompasses methods for inhibiting growth or killing of an insect pest, comprising contacting the insect pest with an insecticidally-effective amount of the combination of an affinity construct of the disclosure and an insecticidal protein, wherein the insecticidal protein corresponds to the insecticidal protein, which the at least one affinity molecule B of the affinity construct of the disclosure is capable of binding to, or is binding to, or is being directed to, or is being designed to bind to.

As used herein, by “controlling an insect pest population” or “controls an insect pest” is intended any effect on an insect pest that results in limiting the damage that the pest causes. Controlling an insect pest includes, but is not limited to, killing the pest, inhibiting development of the pest, altering fertility or growth of the pest in such a manner that the pest provides less damage to the plant, decreasing the number of offspring produced, producing less fit pests, producing pests more susceptible to predator attack or deterring the pests from eating the plant.

In various embodiments methods are provided for controlling an insect pest population resistant to an insecticidal protein, comprising contacting the insect pest population with an insecticidally-effective amount of the combination of an affinity construct of the disclosure, or fragment or variant thereof, and an insecticidal protein or fragment or variant thereof, wherein the insecticidal protein or fragment or variant thereof corresponds to the insecticidal protein, which the at least one affinity molecule B of the affinity construct of the disclosure is capable of binding to, or is binding to, or is being directed to, or is being designed to bind to.

In various embodiments, methods are provided for protecting a plant from an insect pest, comprising expressing in the plant or cell thereof a affinity construct of the disclosure, or fragment or variant thereof, and an insecticidal protein or fragment or variant thereof, wherein the insecticidal protein or fragment or variant thereof corresponds to the insecticidal protein, which the at least one affinity molecule B of the affinity construct of the disclosure is capable of binding to, or is binding to, or is being directed to, or is being designed to bind to.

### Insects

As used herein, the term “insect” encompasses in its broad popular sense all species of the superphylum Panarthropoda (classification Systema Naturae, Brands, S. J. (comp.) 1989-2005. Systema Naturae 2000. Amsterdam, The Netherlands, [http://sn2000.taxonomy.nl/]), including the phyla Arthropoda, Tardigrada and Onychophora; it includes all the different phases of the life cycle of the insect, such as, but not limited to eggs, larvae, nymphs, pupae and adults. In the context of the present disclosure, an insect is a living insect, i.e., that, for example, histological preparations of insects are excluded from the present disclosure.

Preferably, the insect belongs to the phylum Arthropoda (including, but not limited to the orders Archaeognatha, Thysanura, Paleoptera and Neoptera, also ticks, mites and spiders), more preferred to the subphylum Hexapoda, even more preferred to the class Insecta, and most preferred to one of the following orders: Isoptera, Blattodea, Orthoptera, Phthiraptera, Thysanoptera, Hemiptera, Hymenoptera, Siphonaptera, Diptera, Coleoptera and Lepidoptera. Most preferred the insect belongs to one of the families of Crambidae, Noctuidae, Pyralidae, Chrysomelidae, Dynastidae, Elateridae, Melolonthinae, Curcolionidae, Scarabaeidae, Erebidae, Coccinellidae, Mebidae, or Lamiinae.

In various aspects, the insect is considered as a pest. As used herein, “pest” is an organism that is detrimental to humans or human concerns, and includes, but is not limited to agricultural pest organisms, household pest organisms, such as cockroaches, ants, etc., and disease vectors, such as malaria mosquitoes. More preferably, said insect is an agricultural pest organism feeding on agricultural crops like corn, soy or cotton. As used herein, a “living insect” refers to the insect as it occurs in its natural habitat.

The agricultural pest insect preferably is a lepidopteran insect selected from the following insects from the order Lepidoptera: Ostrinia nubilalis (Europen Corn Borer), Diatraea grandiosella (South Western Corn Borer), Helicoverpa zea (Corn Earworm), Agrotis ipsilon (Black Cutworm), Agrotis subterranea (Granulate Cutworm), Agrotis malefida (Palesided Cutworm), Spodoptera frugiperda (Fall Army worm), Spodoptera eridania (Southern Armyworm), Spodoptera albula (Gray-Streaked Armyworm), Spodoptera cosmioides, Spodoptera ornithogalli, Spodoptera exigua (Beet Cutworm), Helicoverpa armigera (Cotton Bollworm), Helicoverpa zea (Corn Earworm), Heliothis virescens (Tobacco budworm), Diatraea saccharalis (SugarCane Borer), Diatraea grandiosella (South Western Corn Borer), Elasmopalpus lignosellus (Lesser CornStalk Borer), Striacosta albicosta (Western bean cutworm), Chrysodeixis includens (Soybean looper), Pseudaletia sequax (Wheat armyworm), Porosagrotis gypaetina, Euxoa bilitura (Potato Cutworm), Pseudaletia unipuncta (True armyworm), Anticarsia gemmatalis (Velvetbean caterpillar), Plathypena scabra (Green cloverworm), Elasmopalpus lignosellus (Lesser CornStalk Borer), Chrysodeixis includens (Soybean looper), Trichoplusia ni (Cabbage Looper) and Peridroma saucia (Variegated Cutworm).

Further preferred the agricultural insect pest is a coleopteran insect selected from the following insects from the order Coleopoptera: Diabrotica virgifera virgifera (Western Corn Rootworm), Diabrotica barberi (Northern Corn Rootworm), Diabrotica speciosa, Diloboderus abderus, Phyllophaga spp (Scarab beetles), Listronotus spp. (Argentine stem weevil), Cerotoma arcuatus, Popillia japonica (Japanese beetle), Colaspis brunnea (Grape colaspis), Cerutoma trifurcata (Bean Leaf Beetle), Epilachna varivestis (Mexican bean beetle), Diabrotica undecimpunctata howardi (Spotted cucumber beetle), Epicauta pestifera (Blister beetles), Popillia japonica (Japanese beetle), Colaspis brunnea (Grape colaspis), Dectes texanus texanus (Soybean stem borer), and Anthonomous grandis (Boll weevil).

Equally preferred the agricultural insect pest is one of the following, but is not limited to, Oscinella frit (Fruit Fly), Myzus persicae (Green Peach Aphid), Rhopalosiphum maidis (Corn Leaf Aphid) and Rhopalosiphum padi (Bird Cherry-Oat Aphid).

It is to be acknowledged that the present disclosure is not limited to the particular nucleic acid molecules, proteins, methodology, protocols, cell lines, genera, and reagents described herein, as such may vary. It is also to be acknowledged that the terminology used herein is for the purpose of describing particular embodiments only and is not intended to be limiting the scope of the present disclosure. The following examples are offered by way of illustration and not by way of limitation.

### EXAMPLES

### Example 1. Identification and Selection of Insect Structures for Immunization Procedures

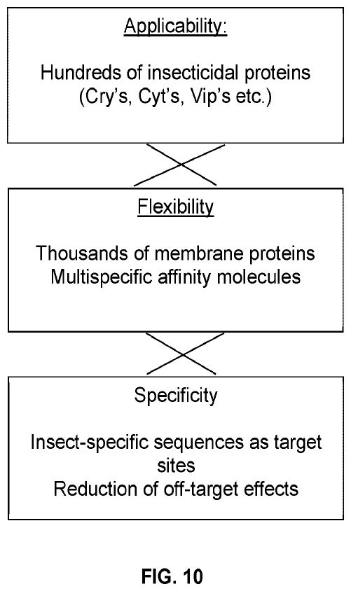

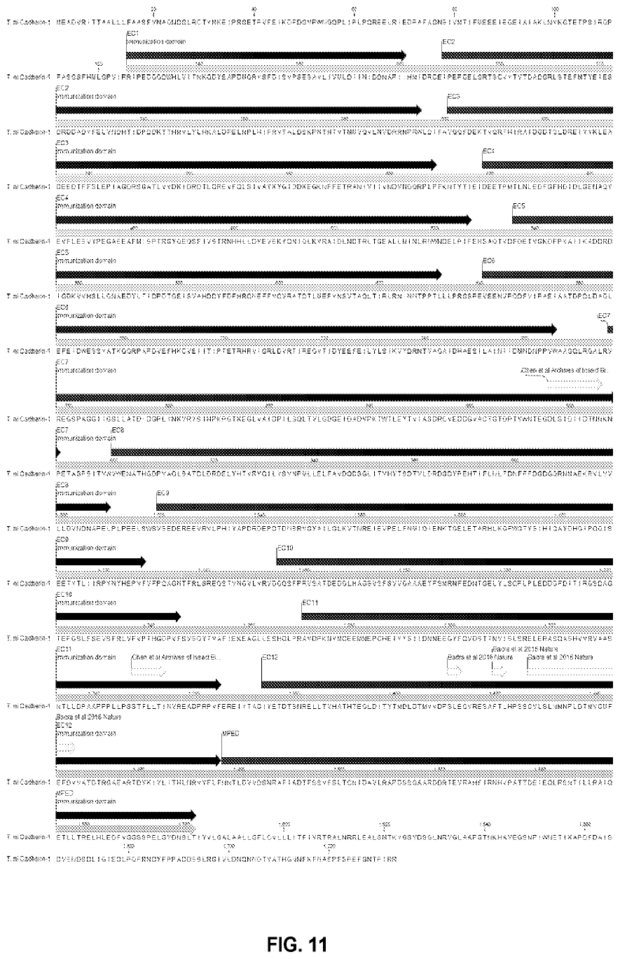

FIG. 10 provides a schematic of the broad approach to and advantages of development of fusion proteins with insecticidal applications. The steps involve selection of target insecticidal proteins and insect gut membrane targets and developing affinity molecules that recognize epitopes on these targets, fusing the affinity molecules such that the insecticidal proteins can be specifically targeted to defined membrane targets.

### Insecticidal Protein for Affinity Molecule Identification

Insecticidal proteins as targets for affinity molecules can be three-domain Cry proteins, such as Cry1Ac or Cry3Ab or Vip3Aa or Cry1F (SEQ ID. NOS. 51, 52, 53 and 34 respectively). Targets can also be domains of such proteins that are known to interact with receptors at the insect gut membrane, such as specific loops in domain 2 of Cry proteins (see Bravo et al. 2013 as example for Cry domains that are involved in binding to membrane proteins). However, the three-dimensional structure of such a purified fragment of the insecticidal protein (e.g. domain 2 loop 1 and 3 of Cry3Ab) might not be the same when compared to the fragment in the native protein. Therefore, using only such partial domains of insecticidal proteins might hamper the identification of affinity molecules. Using native proteins as targets for identification of affinity molecules and the same insecticidal proteins that are mutated in these binding domains is very helpful for the identification of affinity molecules that bind specifically to the natural insect receptor binding domains.

### Cry-Receptors

Insect structures for immunization can be proteins or epitopes of proteins that already serve as natural receptors for conventional Cry proteins (see general part of the description). Cadherins are one class of Cry receptors that localize to intercellular adhesion points (Carthew 2005, Current opinion in genetics & development 15, 358-363) and are abundant in the microvilli of midgut epithelia (Chen et al. 2005, Cell and tissue research 321, 123-129). Homologs of cadherins are identified as Cry binding proteins in many insect species, including several important agricultural pests (Flannagan et al., Insect biochemistry and molecular biology 35, 33-40; Jenkins et al. 2001, BMC biochemistry 2, 12; Jurat-Fuentes and Adang 2006, Biochemistry 45, 9688-9695). The extracellular domain of Cadherins contain cadherin repeats and one membrane-proximal extracellular domain (MPED). These cadherin repeats and the extracellular domains present the binding regions for Cry proteins (Gomez et al. 2001, The Journal of biological chemistry 276, 28906-28912; Dorsch et al. 2002, Insect biochemistry and molecular biology 32, 1025-1036; Hua et al. 2004, The Journal of biological chemistry 279, 28051-28056; Xie et al. 2005, The Journal of biological chemistry 280, 8416-8425; Rahman et al. 2012, Applied and environmental microbiology 78, 354-362; Fabrick et al. 2009, The Journal of biological chemistry 284, 18401-18410). Cadherin sequences were isolated from Spodoptera frugiperda (Fall army worm, FAW, SEQ ID NOS. 1 (DNA), SEQ ID NOS. 2 (protein)); Heliothis virescens (Tobacco budworm, TBW, SEQ ID NOS. 7 (DNA), SEQ ID NOS. 8 (protein)); Helicoverpa armigera (Cotton bollworm, CBW, SEQ ID NOS. 7 (DNA), SEQ ID NOS. 8 (protein)) and Diabrotica virgifera virgifera (Western Corn Root Worm, WCRW, SEQ ID NOS. 5 (DNA), SEQ ID NOS. 6 (protein)). The extracellular domains are used as epitopes for single domain antibody production.

### Membrane Proteins

The binding of Cry proteins to their receptors in insect midguts facilitates insertion and pore formation. One property shared by most Cry receptors is their location at the luminal site of the membrane of insect epithelium midgut cells. It is suggested that creating a membrane-like environment is sufficient to facilitate oligomerization and pore formation of three-domain Cry proteins. This means that targeting Cry proteins to insect gut membrane environments is sufficient to facilitate pore formation of three-domain Cry proteins. Therefore, any membrane-localized protein that can serve as potential Cry receptor could lead to oligomerization and pore formation of Cry proteins, if the Cry proteins are fused to a single domain antibody or a fragment thereof, e.g., the CDR3 loop of an sdAb, raised against luminal epitopes of midgut epithelial proteins. As example for showing the application of this approach, the luminal domains of chitin synthases were used for single domain antibody production. Chitin synthases facilitate the biosynthesis of chitin, which is a dominant molecule of the peritrophic matrix in the midgut of most insect pests. Midgut chitin synthases are expressed during the intermolt stages of feeding larvae and are localized at the apical half of the brush border microvilli formed by the midgut columnar cells (Broehan et al. 2007, The Journal of experimental biology 210, 3636-3643; Zimoch et al. 2002, Cell and tissue research 308, 287-297). The luminal extracellular domains have been used in yeast two hybrid assays for the identification of binding partners (Broehan et al. 2007). The identified proteins also bind in vivo, indicating that expressing these domains in heterologous systems retains their three-dimensional structure. Chitin synthases from Spodoptera frugiperda (Fall army worm, FAW, SEQ ID NOS. 11 (DNA) and SEQ ID NOS. 12 (protein)); Heliothis virescens (Tobacco budworm, TBW); Helicoverpa armigera (Cotton bollworm, CBW, SEQ ID NOS. 9 (DNA) and SEQ ID NOS. 10 (protein)) and Diabrotica virgifera virgifera (Western Corn Root Worm, WCRW) are isolated. The extracellular domains are used as epitopes for single domain antibody production.

### Other Criteria for Selection of Insect Target Proteins

Cry-susceptible insects get resistant by gaining mutations in Cry-receptor proteins. Such resistances can be detrimental for using other functional Cry proteins (e.g., in stacks) if their mode of action is based on the same receptor binding sites. While fitness costs can greatly influence the rate of resistance evolution, mutations in the target proteins leading to Cry resistance are supposed to be not essential for insect survival. However, by targeting Cry proteins to physiologically essential membrane proteins, resistance establishment can be highly reduced since the fitness of resistant insects is very low. Therefore, the present inventors have selected specific gut epithelial membrane-bound insect structures for immunization procedures that are highly relevant for insect survival, e.g., Chitin synthase 2 (Arakane et al. 2005, Insect molecular biology 14, 453-463).

Target proteins for immunizations might also be proteins that interact with known Cry receptors. These interacting proteins might be membrane-bound or membrane-integral proteins or might be cytosolic proteins. Creating affinity to these target proteins via the single domain antibody technology is considered to increase the probability of (1) interactions between Cry proteins and their natural receptors, and (2) locating the Cry proteins in the vicinity of the plasma membrane.

### Toxin Protein Production

The Cry1a gene (SEQ ID NOS. 45) was isolated from the B. thuringiensis var. thuringiensis T01-328 strain, and the complete gene (2160 bp) was cloned into the pET28a(+) expression vector (Bergamasco et al. 2013, J Invertebr Pathol 112, 152-158). The Vip3Aa gene (SEQ ID NOS. 47) was isolated from the B. thuringiensis HD-1 line, and the complete gene (2350 bp) was cloned into the pET SUMO expression vector. Cry3Aa gene (SEQ ID NOS. 46) was isolated from B. thuringiensis. The expression vectors added a polyhistidine tag (6 His) to the end of the recombined genes for protein detection and purification. The vectors containing the genes were used to transform competent E. coli BL21(DE3) cells by thermal shock (Hanahan 1983, J Mol Biol 166, 557-580) to induce recombinant gene expression.

Cry1a and Vip3Aa expression was induced by inoculating a pre-culture containing 20 ml LB media and 50 μg/ml kanamycin with a single colony from one of the clones containing the expression vector with the specific gene. The culture was grown at 37° C. and agitated at 250 rpm for 16 h. The pre-culture was transferred to 200 ml of LB media and 50 μg/ml kanamycin and agitated until an OD₆₀₀ of 0.6 was reached. IPTG was then added to a final concentration of 1 mM (Vip3Aa) or 5 mM (Cry1a) to induce expression. The culture was maintained at 25° C. (Vip3Aa) or 30° C. (Cry1a) for 24 h with agitation (190 rpm). Cell lysis and solubilization of the proteins were performed as described by Bergamasco et al., 2013. Gene expression was confirmed by resolving the total protein on a 10% SDS-PAGE gel stained with Coomassie Blue and by Western Blot using an antihistidine antibody (Sigma Aldrich). Lysate from E. coli BL21 (DE3) without the gene inserts was used as a negative control. Lysates containing Cry1a (approximately 81 kDa) (Bergamasco et al. 2013) in the lysate were quantified by densitometry via the Bionumerics software (Applied-Maths) and a bovine serum albumin (BSA) standard curve before use in the bioassays and BBMV binding studies (Bergamasco et al. 2013; primary and secondary literature).

Another source for a protocol is: Production of a Bt toxin standard and development of a measuring procedure to assess the amount of the toxin in Bt maize. State Teaching and Research Centre for Agriculture, Viticulture and Horticulture (SLFA), Neustadt. Research Project from BMBF, Förderkennzeichen 0312631 C (2001-2004).

### Example 2: Identification of Nanobodies

This disclosure also contemplates immune V_{H}H libraries obtained from naive, semisynthetic, or synthetic V repertoires raised against insect target proteins (Goldman et al., 2006, Anal Chem 78, 8245-8255; Monegal et al. 2009, Protein Eng Des Sel 22, 273-280). The term “raised against” as used herein refers to the specific polypeptide sequence that was used as an antigen to raise affinity molecules for example (but not restricted to) antibody, nanobody, V_{H}H, sdAb etc. Target insect antigen or toxin specific nanobodies can be retrieved from immune or other libraries by phage display or any other selection protocol, including bacterial display, yeast display, intracellular 2 hybrid selection (Pellis et al. 2012, Arch Biochem Biophys 526, 114-123; Zolghadr et al. 2008, Mol Cell Proteomics 7, 2279-2287), ribosome display (Yau et al. 2003, J Immunol Methods 281, 161-175), and others (as mentioned in Muyldermans 2013, Annual review of biochemistry 82, 775-797).

### V_{H}H Library Construction and Panning

Lymphocytes can be isolated by Ficoll gradient centrifugation from blood of immunized llamas and total RNA can be isolated, from which cDNA can be prepared. This cDNA can be used as template in a PCR reaction using e.g. primers annealing to the common CH2 exon of the heavy chain llama immunoglobulins and to the leader sequence (5′-GTCCTGGCTGCTCTTCTACAAGG-3′ (SEQ ID NOS. 41) and 5′-GGTACGTGCTGTTGAACTGTTCC-3′ (SEQ ID NOS. 42), Monegal et al., 2009). PCR products can be separated on an agarose gel and V_{H}H products (about 600 bp) can be purified. These PCR products can be used as a template for a nested PCR using degenerated primers (e.g., PCR-2 primers: 5′-CCAGCCGGCCATGGCTGAKGTBCAGCTGGTGGAGTCTGG-3′ and 5′-GGACTAGTGCGGCCGCGTGAGGAGACGGTGACCWGGGT-3′ and PCR-3 primers: 5′-AACATGCCATGACTCGCGGCTCAACCGGCCATGGCTGAKGTBCAGCTGCAGGC GTCTGGRGGAGG-3′ and 5′-GTTATTATTATTCAGATTATTAGTGCGGCCGCTGGAGACGGTGACCWGGGTCC-3′; see also Monegal et al. 2009). The PCR product (about 400 bp) can be cloned into suitable vectors (e.g., pHEN4 vector, Arbabi Ghahroudi et al. 1997, FEBS Lett 414, 521-526).

The cloned V_{H}H library can be expressed preferably on a phage and panned on an antigen (e.g., insect protein) that is immobilized in wells of microtiter plates by passive adsorption (or other methods). The antigen can also be biotinylated and immobilized on streptavidin-coated solid supports (Hoogenboom, 2005, Nat Biotechnol 23, 1105-1116). Two to three rounds of panning are normally sufficient to enrich the clones so that individual clones can be screened for production of antigen-specific nanobodies (e.g., against insect midgut proteins) in a standard enzyme-linked immunosorbent assay (ELISA). After panning (phage display (Hammers and Stanley, 2014, J Invest Dermatol 134, e17), the entire antigen binding fragment of nanobodies (˜360 bp) is easily amplified by PCR in one single amplicon (e.g., primers annealing to the common CH2 exon of the heavy chain llama immunoglobulins and to the leader sequence can be used (5′-GTCCTGGCTGCTCTTCTACAAGG-3′ (SEQ ID NOS. 41) and 5′-GGTACGTGCTGTTGAACTGTTCC-3′, (SEQ ID NOS. 42) Monegal et al. 2009). Small libraries of ˜100 individual transformants are representative of the immune V_{H}H repertoire of B cells present in a Camelid blood sample of ˜50 ml. The amino acid sequences of the Nanobodies can be obtained from nucleotide sequencing of the ELISA-positive clones.

### Example 3: Determination of Binding Efficiency

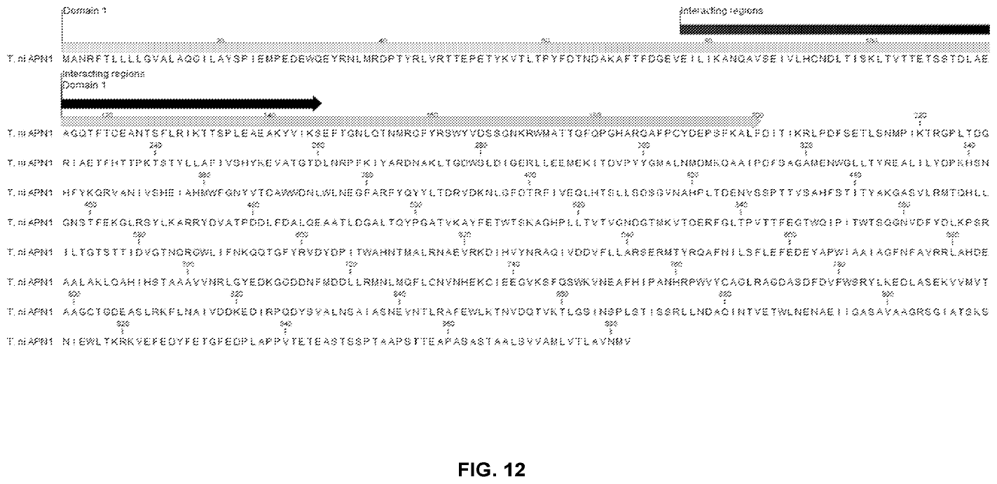

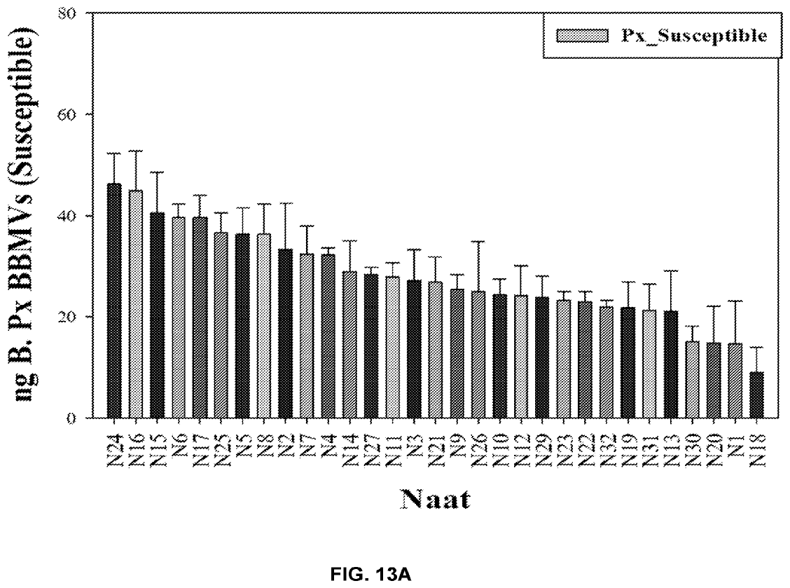

Bacterial cells containing V_{H}H-containing plasmids can be infected with helper phages (e.g., KM13). Phage particles can then be isolated from culture supernatant and used for panning against purified soluble protein constructs. Isolated antigens can be bound via Tags (e.g., GST or Fc fragments) on coated on immunotubes, which are then incubated with the phages. Panning is done with peptides, domains or sub-domains of target insect midgut proteins that bind to insecticidal proteins. In the case of Cadherin, these domains include CR 7, 11 and 12 (see FIG. 11 as an example for T.ni cadherin). In addition, the affinity molecules are panned against peptides, domains or sub-domains within the extracellular domain of cadherins that are not usually bound by the toxin, e.g., CR 8-11 or the MPED domain (see FIG. 11) and therefore do not interfere with binding of Cadherin to natural binding sites. The same principle is applied to other natural receptors of toxins (e.g., Cry proteins) such as APN1. Cry1C, for example interacts with a specific region in domain 1 of APN1 (Kaur et al. 2014, Process Biochemistry 49, 688-696). Domain 1 of APN1 could be used for immunization, while peptides from the specific binding region, or peptides outside of the binding region in domain 1 of APN1 could be used for panning, since this will allow new toxin-receptor interactions, without interfering with conventional binding interactions (See FIG. 12).

After several rounds of washing, phages can be eluted and used to infect bacterial cells (e.g., TG1). After infection with helper cells (e.g., KM13) and incubation, phage particles can be isolated and used for additional rounds of panning. Screening of V_{H}Hs can be performed via ELISA. For this, Antigens (toxins or insect epitopes) can be bound to reaction tubes and incubated with periplasmic lysates of V_{H}H-containing cultures and binding can be determined calorimetrically (e.g., by using ABTS and measuring absorbance at 405 nm). Clones with unique V_{H}H sequences can then be cloned into vectors containing specific tags (e.g., His-tag) and purified via affinity chromatography.

For example, nanobodies against insect derived proteins with kinetic kₒₙ and k_{off} rate constants in the ranges of 10⁵ to 10⁶ M⁻¹s⁻¹, and 10⁻² to 10⁻⁴ s⁻¹ might be used, however, higher kinetic rate constants are also preferred if a less efficient or transient binding to a specific insect receptor or other protein is needed.

In vitro affinity maturation approaches, such as error-prone PCR, spiked mutagenesis combined with ribosome display (Yau et al. 2005) and Ala scanning-based mutations to identify the critical amino acids for antigen recognition might be used to improve the stability of the domain and/or the affinity for the cognate antigen (Koide et al, 2007, J Mol Biol 373, 941-953). Alternatively, carefully selected mutations at the edge of the paratope can be introduced to affect antigen-Nanobody kinetic and equilibrium affinity values. When combined with a multivariate analysis of the parameterized quantitative descriptors of the mutations and buffers, these methods can be used to propose a quantitative predictive algorithm that models the affinity parameters of all other possible mutants at those positions.

The method described herein includes immunization of Dromedaries or Llamas with proteins, peptides, protein fragments or other chemical structures from insect midgut or other insect tissues as well as toxins. V_{H}H libraries can be then obtained from immunized dromedary (or Llama) in the form of phage display vectors. From these immune libraries the antigen-specific V_{H}Hs can be selected (SEQ ID NOS. 28 provides an amino acid sequence of V_{H}H domain from Dromedary germline, SEQ ID NOS. 29 provides an example of 2 V_{H}H domains from Dromedary germline, linked by a linker). Small recombinant monomeric nanobodies (15 kDa, about 110 amino acid residues) can be selected that bind the target with 1 nM to 1 mM affinity. Reduced affinity might be preferable if the nanobody-insecticidal agent needs to bind with less specificity or if the interaction with target proteins needs to be transient. Transient interactions might help to concentrate the nanobody-insecticidal agent at specific structures in the insect, including the gut epithelium, as for example in the case of nanobody-Cry combinations, where Cry protein processing and oligomerization lead to the formation of pores in the membrane, finally impairing insect performance. Because of their small size, nanobodies are preferred over conventional antibodies because they can bind specific epitopes that are less immunogenic for conventional antibodies, such as the active sites of enzymes (Muyldermans, 2013). Therefore, nanobodies can target areas or structures that are not accessible to conventional antibodies.

Straightforward identification of antigen-binding V_{H}Hs after immunizing a camelid includes cloning the V_{H}H repertoire of B cells circulating in blood and panning by phage display (Nguyen et al. 2001, Adv Immunol 79, 261-296). The sequence variability within V domains is localized in three hypervariable (HV) regions surrounded by more conserved framework (FR) regions. The folded V domain comprises nine β-strands (A-B-C-C-C-D-E-F-G), organized in a four-stranded β-sheet and a five-stranded β-sheet, connected by loops and by a conserved disulfide bond between Cys23 and Cys94, packed against a conserved Trp. In this architecture, the HV regions are located in the loops H1 to H3 that connect the B-C, the C-C, and the F-G strands, respectively, and that cluster at the N-terminal end of the domain forming a continuous surface, which is complementary to the surface of the epitope, hence its name, complementarity-determining region (CDR, see FIG. 7).).

### Example 4: In Vitro Affinity Maturation

In vitro affinity maturation approaches, such as error-prone PCR, spiked mutagenesis combined with ribosome display (Yau et al. 2005, J Immunol Methods 297, 213-224) and Ala scanning-based mutations to identify the critical amino acids for antigen recognition might be used to improve the stability of the domain and/or the affinity for the cognate antigen (Koide et al. 2007, J Mol Biol 373, 941-953). Alternatively, carefully selected mutations at the edge of the paratope can be introduced to affect antigen-V_{H}H kinetic and equilibrium affinity values. When combined with a multivariate analysis of the parameterized quantitative descriptors of the mutations and buffers, these methods can be used to propose a quantitative predictive algorithm that models the affinity parameters of all other possible mutants at those positions.

### Example 5: Construction of Multispecific V_{H}Hs

### Linker

Affinity molecules (or fragments thereof) can be fused directly or by using a flexible linker which does not interfere with the structure and function of the proteins (or fragments thereof) to be linked. Said flexible linkers are for instance those which have been used to fuse the variable domains of the heavy and light chain of immunoglobulins to construct a scFv, those used to create bivalent bispecific scFvs or those used in immunotoxins (see, for example, Huston et al. 1992; Takkinen et al. 1991). Linkers can also be based on hinge regions in antibody molecules (Pack et al. 1993, Biotechnology (NY) 11, 1271-1277; Pack and Plückthun 1992, Biochemistry 31, 1579-1584) or on peptide fragments between structural domains of proteins.

A linker can be designed as a flexible GGGS-linker of three distinct lengths (9, 25, 35 amino acids containing glycine for flexibility and serine for solubility), as fusion head-to-tail with a 9 amino acid glycine/serine linker (preferred option) or as hinge-sequence added to the 3′ extremity of an affinity molecule. Some exemplary linker sequences are provided in SEQ ID NOS 54-65.

### Example 6: Application of Multispecific Affinity Molecules

One of the main aspects of the present disclosure is to apply the purified multispecific affinity molecule to the plant (e.g., by spraying), together with the insecticidal protein(s) for which affinity was generated. Upon feeding on the plant, an insect would then take up the affinity molecule(s) as well as the insecticidal protein(s). The oligomerization capacity and therefore pore formation activity of the insecticidal protein would be enhanced through higher binding capacities to insect receptors via the multispecific affinity molecule.

Alternatively, multispecific affinity molecules can be easily expressed in plants also expressing the insecticidal protein. Affinity molecules such as the V_{H}H's can be readily expressed by transformed plants (Ismaili et al. 2007, Biotechnol Appl Biochem 47, 11-19). Expression in transgenic plants can be done using constitutively active promotors (e.g. 35S promotor or Ubiquitin promotors) or using specific promotors that allow increasing toxin activity in areas that are attacked by the target insects or that can be induced via external cues (e.g. chemically-inducible promotors, heat-inducible promotors).

The invention also includes applying the insect protein to which the affinity molecule is intended to bind to. The insect protein that is co-applied with the affinity molecule might also be equipped with a tag that is specific for a V_{H}H. Such tags have been described previously (De Genst et al. 2010, J Mol Biol 402, 326-343). However, these tags could be any protein or amino acid sequence, for which a specific antibody or V_{H}H can be produced. The multispecific affinity molecule can also be introduced to the plant by other means, such as viral vectors, bacteria, injection, grafting, spraying and others.

### Example 7: Insect Specificity Via Exchanging V_{H}H or CDR3

V_{H}H against epitopes from different insects are raised (see Example 1: Insect structures for immunization procedures). By exchanging the V_{H}H or CDR3 domain between different multispecific affinity molecules, insecticidal proteins are targeted to the membranes of previously non-susceptible insects, thereby creating toxicity to these insects (see FIGS. 4 and 5). Table 2(A-D) provides examples of the use of the approaches for increasing activity of insecticidal proteins and for creating activity of insecticidal proteins in insects, for which the insecticidal protein was not yet active.

Specifically, Table 2A provides the relative activity of native insecticidal proteins against the target insects (FAW=fall armyworm (Spodoptera frugiperda), TBW=Tobacco budworm (Heliothis virescens), CBW=Cotton bollworm (Helicoverpa armigera), WCRW=Western corn rootworm (Diabrotica virgifera virgifera), CL=Cabbage looper=Trichoplusia ni, Question mark indicates that activity has not been described). As seen from Table 2, different native Cry proteins show varying degrees of activity against different target insects. For instance, Cry3Ac is highly active against CBW but shows no activity against WCRW and CL, and mild activity against FAW. Similarly, Cry3Aa shows no activity against FAW, TBW and CBW but is highly active against WCRW.

TABLE 2A
Relative activity of native insecticidal
proteins against the target insects

| Target Insect | Cry1Ac | Cry3Aa | VIP3Aa |
|---|---|---|---|
| FAW | mildly active | not active | active |
| TBW | active | not active | active |
| CBW | highly active | not active | active |
| WCRW | not active | highly active | not active |
| CL | active | not active | highly active |

However, by exchanging the V_{H}H or CDR3 domain between different multispecific affinity molecules, Cry proteins can be made active against previously non-susceptible species.

Table 2B provides potential activity of Cry1Ac V_{H}H/CDR3_{Cry1Ac}-V_{H}H/CDR3_{insect-target} combinations. V_{H}H/CDR3xxx represents either V_{H}H or CDR3 loop domains raised against Cry1Ac (V_{H}H/CDR3_{Cry1Ac}) or insect-specific epitopes (V_{H}H/CDR3_{insect-target}).

TABLE 2B
Activity of Cry1Ac V_{H}H/CDR3_{Cry1Ac}-V_{H}H/CDR3_{insect-target} combinations.
Bold letters indicate changes in activity, when compared to Table 2A.

| | Cry1Ac + | Cry1Ac + | Cry1Ac + | Cry1Ac + | Cry1Ac + |
|---|---|---|---|---|---|
| Target | V_{H}H/CDR3_{Cry1Ac} | V_{H}H/CDR3_{Cry1Ac} | V_{H}H/CDR3_{Cry1Ac} | V_{H}H/CDR3_{Cry1Ac} | V_{H}H/CDR3_{Cry1Ac} |
| Insect | V_{H}H/CDR3_{FAW} | V_{H}H/CDR3_{TBW} | V_{H}H/CDR3_{CBW} | V_{H}H/CDR3_{WCRW} | V_{H}H/CDR3_{CL} |
| FAW | highly active | mildly active | mildly active | mildly active | mildly active |
| TBW | active | highly active | active | active | active |
| CBW | highly active | highly active | highly active | highly active | highly active |
| WCRW | not active | not active | not active | highly active | active |
| CL | active | Active | active | active | highly active |

As seen from Table 2B, it is conceivable that Cry1Ac, that previously showed low activity against FAW, can now be highly active against FAW when used in conjunction with V_{H}H/CDR3_{Cry1Ac}-V_{H}H/CDR3_{FAW}. Surprisingly, WCRW, against which Cry1Ac has no activity, can be made susceptible to Cry1Ac, when used in combination of V_{H}H/CDR3_{Cry1Ac}-V_{H}H/CDR3_{WCRW}.

Table 2C provides activity of Cry3Aa V_{H}H/CDR3_{Cry3Aa}-V_{H}H/CDR3_{insect-target} combinations. V_{H}H/CDR3xxx represents either V_{H}H or CDR3 loop domains raised against Cry3Aa (V_{H}H/CDR3_{Cry3Aa}) or insect-specific epitopes (V_{H}H/CDR3_{insect-target}).

TABLE 2C
Activity of Cry3Aa V_{H}H/CDR3_{Cry3Aa}-V_{H}H/CDR3_{insect-target} combinations.
Bold letters indicate changes in activity, when compared to Table 2A.

| | Cry3Aa + | Cry3Aa + | Cry3Aa + | Cry3Aa + | Cry3Aa + |
|---|---|---|---|---|---|
| Target | V_{H}H/CDR3_{Cry3Aa} | V_{H}H/CDR3_{Cry3Aa} | V_{H}H/CDR3_{Cry3Aa} | V_{H}H/CDR3_{Cry3Aa} | V_{H}H/CDR3_{Cry3Aa} |
| Insect | V_{H}H/CDR3_{FAW} | V_{H}H/CDR3_{TBW} | V_{H}H/CDR3_{CBW} | V_{H}H/CDR3_{WCRW} | V_{H}H/CDR3_{CL} |
| FAW | highly active | not active | mildly active | mildly active | mildly active |
| TBW | not active | highly active | active | active | active |
| CBW | not active | not active | highly active | highly active | highly active |
| WCRW | highly active | highly active | not active | highly active | active |
| CL | ? | ? | active | active | highly active |

Similarly, Table 2D provides activity of Vip3Aa fusion protein. V_{H}H/CDR3_{Vip3A}-V_{H}H/CDR3_{insect-target} combinations. V_{H}H/CDR3xxx represents either V_{H}H or CDR3 loop domains raised against Vip3A (V_{H}H/CDR3_{VIP3Aa}) or insect-specific epitopes (V_{H}H/CDR3_{insect-target}).

TABLE 2D
Activity of Cry3Aa Vip3Aa fusion protein. Bold letters indicate
changes in activity, when compared to Table 2A.

| | Vip3Aa + | Vip3Aa + | Vip3Aa + | Vip3Aa + | Vip3Aa + |
|---|---|---|---|---|---|
| Target | V_{H}H/CDR3_{ViP3Aa} | V_{H}H/CDR3_{ViP3Aa} | V_{H}H/CDR3_{ViP3Aa} | V_{H}H/CDR3_{ViP3Aa} | V_{H}H/CDR3_{ViP3Aa} |
| Insect | V_{H}H/CDR3_{FAW} | V_{H}H/CDR3_{TBW} | V_{H}H/CDR3_{CBW} | V_{H}H/CDR3_{WCRW} | V_{H}H/CDR3_{CL} |
| FAW | highly active | active | active | active | active |
| TBW | Active | highly active | active | active | active |
| CBW | Active | active | highly active | active | active |
| WCRW | not active | not active | not active | highly active | not active |
| CL | ? | ? | active | active | highly active |

As seen in all these cases, combination of V_{H}H or CDR3 that specifically recognize and bring together toxins to their insect specific targets can be a powerful tool to generate greater toxicity towards insect pests and indeed to increase the repertoire of insects against which these toxins can be effectively used.

### Example 8: Overcoming Resistance in Insects

One of the most common insect resistance mechanisms is based on mutations in domains of proteins that serve as receptors for insecticidal proteins. However, by targeting the insecticidal protein to other domains of known receptors, or to new proteins that are not yet associated with the insecticidal protein, binding of the insecticidal proteins to said other domains of known receptors or to new proteins is able to break this type of resistance.

To test this Cry1Ac was provided with V_{H}H/CDR3_{Cry1Ac}-V_{H}H/CDR3_{Chitin synthase} fusion protein between V_{H}H or CDR3 loops of V_{H}H raised against Cry1Ac fused with V_{H}H or CDR3 loops of V_{H}H raised against T. ni chitin synthase 2. Wild type and two strains of Cry1Ac-resistant strains of Trichoplus ni (CL=Cabbage looper) were used.

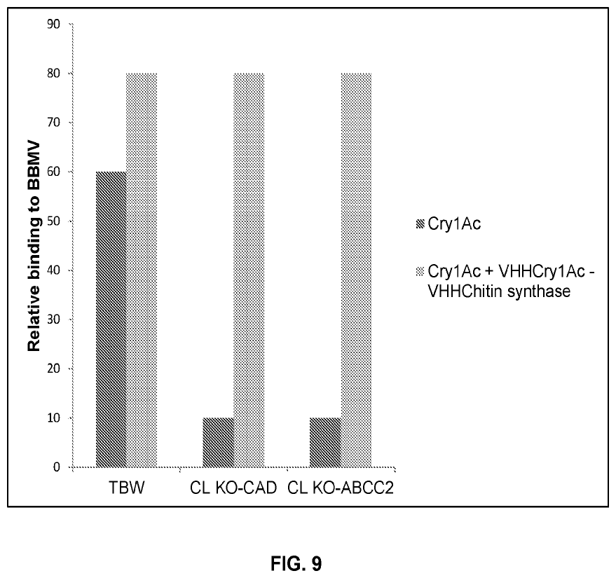

Quantitative data for of these experiments are shown in FIG. 9. Targeting Cry1Ac to insect-specific extracellular luminal domains of the gut chitin synthase overcomes resistance in both mutants (see Table 3). This provides further evidence that using the methods disclosed herein, insects previously resistant to these toxins can be made susceptible.

TABLE 3
Targeting Cry1Ac to luminal chitin synthase epitopes using
V_{H}H or CDR3 loops creates susceptibility in two resistant
strains of T. ni. Bold letters indicate change in activity
when compared to native Cry1Ac toxins in Table 2A.

| | | Cry1Ac + V_{H}H/CDR3_{Cry1Ac}- |
|---|---|---|
| Target insect | Cry1Ac | V_{H}H/CDR3_{Chitin}_synthase |
| CL | Active | highly active |
| CL_{KO-CAD} | not active | highly active |
| CL_{KO-ABCC} | not active | highly active |

### Example 9: Protein Expression Analysis and Feeding Assays

Once the multispecific affinity proteins are cloned into appropriate expression vectors (e.g., vectors that contain a GST-tag, such as pEMBO) the recombinant expression plasmid can be transformed into E. coli strains (e.g., BL21). After appropriate growth in LB medium (e.g., at 37° C. to reach OD_{600 0.5-0.8}) the expression can be induced by IPTG (e.g., for 3h at 37° C.) and the bacteria can be gained by centrifuging and washing (e.g., with 0.5% NaCl). The bacteria can be mixed and homogenized, and aliquot of the centrifuged supernatant can be treated with loading buffer (boiling for 3-5 min) and protein expression and size can be analyzed SDS-PAGE electrophoresis.

When expression is appropriate, the supernatant mentioned above can be purified with specific kits (e.g., GST-Bind™, Novagen) and analyzed again via SDS-PAGE electrophoresis. After that protein concentration can be determined. Bioassays to determine LC₅₀ values are described elsewhere (e.g., Ibargutxi et al. 2006, Appl Environ Microbiol 71(1): 437-442).

### Leaf Disc Assays

Leaf punches from a young, fully expanded leaf can be collected using a small paper punch. Leaf punches will not include the leaf midrib. The paper punchers used are cleaned with ethanol between sampling each individual leaf. 128-well assay trays (Bio-Serv) can be half-filled with a 1.5% agar solution (+ perhaps a fungicide). The agar is allowed to harden, and the trays are then be wrapped in plastic and stored at approximately 5° C. Trays will be allowed to reach room temperature before use. Leaf punches are dipped into insecticidal spray formulation and allowed to dry. One leaf punch is placed in each of the wells. One neonate larvae of the appropriate species are placed into each well with a small camel-hair brush. Only healthy, moving neonates are used in the assay. After infestation, wells are sealed with Bio-Serv 16 cell covers. Trays containing larvae are held at 25° C. with 16:8 L:D and 65±5% relative humidity for up to 5 days. The percent leaf area consumed in each well is recorded 3-5 days post-infestation. The actual number of days post-infestation is also recorded. The number of alive and dead insects in the wells per experimental unit is recorded on the same day as the leaf area assessment. Moribund larvae are considered dead. Mortality and weight of larvae are recorded. Other assays are described by Niu et al. 2013, PLoS One 8, e72988; and Olsen and Daly 2000, J Econ Entomol 93, 1293-1299).

### Example 10: Expression of Multispecific Affinity Proteins in Plants and Bioassays

One aspect of the disclosure is the transformation of plants with genes encoding the multispecific affinity proteins. The transformed plants are resistant to attack by the target pest, when co-expressed or treated with the toxin. The coding sequence of positively tested multispecific affinity proteins is cloned into appropriate vectors for plant transformation (e.g., pBR322, pUC series, M13 mp series, etc.). The resulted plasmid is used for transformation into E. coli. The transformed E. coli cells are harvested lysed, and plasmid is recovered. After sequence analysis (electrophoresis, digestion analysis, sequencing), the plasmid is used for stable integration into plants. Techniques for plant transformation include (but are not limited to) transformation with T-DNA using Agrobacterium tumefaciens or Agrobacterium rhizogenes as transformation agent, fusion, injection, biolostics (microparticle bombardment), or electroporation as well as other possible methods. The Agrobacterium cells transformed with the appropriate plasmid (e.g., pLHBA, pZFN) containing the insecticidal fusion protein are used for the transformation of plant cells. Plant explants or calli are cultivated with the transformed Agrobacterium strains and whole plants are then regenerated from the infected plant material (for example, pieces of leaves, segments of stalks, roots, but also protoplasts or suspension-cultivated cells) in a suitable medium, which may contain antibiotics or biocides for selection. The plants obtained are then tested for the presence and expression of the multispecific affinity. The transgenic plants are used for insect bioassays. Tissues used for insect feeding depend on the target insect. Western corn rootworm (WCR, Diabrotica virgifera), for example, can be obtained as eggs and used to infect roots of transgenic and control plants. For this purpose, the soil around the plants is infected with approximately 150-200 WCR eggs and the insects are allowed to feed for 2 weeks, after which a root damage rating can be given to each plant (see Oleson et al. 2005 J Econ Entomol 98, 1-8 for details).

### Example 11

### Identification of Candidate Fall Armyworm Receptor Targets

The goal of this experiment was to identify gut membrane proteins to be used as targets in development of bispecific affinity molecules for control of fall armyworm (FAW, Spodoptera frugiperda). The rationale for target selection was based on identifying proteins with putative high abundance on the surface of midgut cells, with epitopes available on the extracellular surface that were not susceptible to cleavage by phospholipases (avoiding GPI-anchored proteins, “GPI” means glycosylphosphatidylinositol) to promote interaction with affinity molecules and preventing resistance by cleavage and release of target. Presence on the microvillar membranes of midgut cells of FAW was examined by proteomic analysis of midgut brush border membrane vesicle (BBMV) proteins (Silva et al. 2013). Tryptic peptides derived from solubilized BBMV proteins were identified through nano-liquid chromatography coupled to tandem mass spectrometry (nanoLC/MS/MS) and annotated using the FAW TR2012b transcriptome from the Bioinformatics Platform forAgroecosystem Arthropods (BIPAA) and the NCBlnr Insecta databases. This proteomic approach also allowed relative quantification of BBMV proteins using the Normalized Spectral Abundance Factor (NASF) quantitative method (Zhang et al. 2010), which considers the normalized spectra and protein length.

Samples of FAW BBMV proteins were prepared and quantified as described elsewhere (Jakka et al. 2016) and solubilized in 1% SDS at room temperature. Solubilized samples were shipped to MS Bioworks (Ann Arbor, MI) for further analysis and processing. Samples (10 μg) were loaded onto a 10% Bis-Tris SDS-PAGE gel (Novex®, Invitrogen) and resolved for approximately 2 cm into the gel. The gel was stained with Coomassie and the area containing the BBMV proteins excised and processed for trypsin digestion, as follows. The gel was first washed with 25 mM ammonium bicarbonate followed by acetonitrile, and then reduced with 10 mM dithiothreitol at 60° C. followed by alkylation with 50 mM iodoacetamide at room temperature. The proteins were digested with trypsin (Promega) at 37° C. for 4h and reactions quenched with formic acid. The supernatant was analyzed directly without further processing.

Tryptic digests were analyzed by nano LC/MS/MS with a Waters™ NanoAcquity HPLC system interfaced to a Q Exactive™, from ThermoFisher. Peptides were loaded on a trapping column and eluted over a 75 μm analytical column at 350 nL/min; both columns were packed with Luna C18 resin (Phenomenex™). A 1 h gradient was employed. The mass spectrometer was operated in data-dependent mode, with MS and MS/MS performed in the Orbitrap at 70,000 FWHM resolution and 17,500 FWHM resolution, respectively. The fifteen most abundant ions were selected for MS/MS. Data were searched using a local copy of Mascot software (Matrix Science) and parsed into the Scaffold software (Proteome Software) for validation and filtering to create a non-redundant list per sample. Data were filtered using a minimum protein value of 99%, a minimum peptide value of 95.5% (PeptideProphet scores, using software from Institute for Systems Biology) and requiring at least two unique peptides per protein.

Searches for matching proteins were performed against an in silico generated FAW proteome and the NCBI (National Center for Biotechnology Information) nr Insecta protein database. The Blast2GO™ v.5.1.13 software (from Biobam) was used to convert all the contigs generated after translating the FAW TR2012b transcriptome in all 6 potential frames to their longest ORF protein sequences. The resulting file (containing 7,785 sequences) was blasted, mapped and annotated in Blast2GO. Proteins containing “membrane” as part of its GO name were then selected and used to manually annotate identified FAW BBMV proteins. The list of proteins identified by matching to NCBInr Insecta was manually curated to eliminate ribosomal and organelle proteins. The lists of identified FAW BBMV proteins by matching to NCBInr Insecta or the translated TR2012b transcriptome were then ranked based on protein abundance using the NASF method (Zhang et al., 2010) and predicted membrane topology as predicted by the TOPCONS consensus prediction tool (https://topcons.cbr.su.se/).

In the list of proteins identified by searching the TR2012b transcriptome, 587 proteins were detected. This list was reduced to 95 proteins, which were selected after increasing probability cutoff and manually deleting proteins not predicted to be associated with the membrane based on GO terms. A second list of 499 proteins was derived using the NCBInr Insecta database for searches. This list was reduced to 345 proteins after manually deleting proteins predicted to be in intracellular organelles and ribosomal proteins.

### First Identified Protein:

Based on the critical role of ABC transporters in the activity of some Cry toxins, topologically similar transmembrane proteins with extracellular loops of relevant length were considered as target candidates. Among this group, a protein containing domains from the SLC6 (Solute Carrier Family 6,) family of nutrient transporters expected to direct uptake of nutrients from the gut lumen was identified as sodium-dependent nutrient amino acid transporter 1-like (NAAT) protein. The second extracellular loop in this protein was predicted to consist of 57 aa and used to develop nanobodies. The sequence of the antigenic region of NAAT protein and the full-length protein is shown in SEQ ID NOS. 30 and 31 respectively.

### Second Identified Protein

Resistance to Cry1 toxins is linked to mutations in cadherin in several insects (Rahman, et al. 2012, Park and Kim 2013), yet cadherin is not a functional Cry1F or Cry1Ab toxin receptor in FAW as demonstrated in genetic knockouts not affected in Cry1F susceptibility (Zhang, et al. 2020). Consequently, re-targeting Cry1F to FAW cadherin could allow for the progression of the toxin mode of action. Thus, a FAW cadherin region containing the Cry toxin and membrane proximal domains in other lepidopterans and shown to enhance toxicity in FAW (Rahman et al. 2012) was also selected as a potential target for nanobody development. The sequence of the antigenic region and full-length protein is provided in SEQ ID NOS. 35 and 2 respectively.

### Third Identified Protein

As observed in work with lepidopteran BBMV (McNall and Adang, 2003; Krishnamoorthy, et al. 2007; Pauchet, et al. 2009; and Tiewsiri and Wang, 2011), searches with both protein databases identified V-ATPase complex subunits as very abundant proteins in FAW BBMV. Notably, these protein complexes are expected to localize mostly to goblet cells (Wieczorek, et al. 2009), and their detection probably indicates contaminant proteins. Out of the three protein subunits (a, e and c) part of the integral membrane subunit (Vₒ) protein complex of the V-ATPase, subunit a was predicted to include a lengthy region exposed to the extracellular fluid and thus was selected as candidate for nanobody development. The sequence of the antigenic region and full-length protein are provided in is shown in SEQ ID. NOS. 32 and 33 respectively.

### Fourth Identified Protein

Based on the critical role of ABC subfamily C2 (ABCC2) transporters as a receptor for Cry1Fa toxin in FAW (Banerjee, et al. 2017), a member of ABC protein family 1 detected as relatively abundant in FAW BBMV was selected as candidate target. In addition, considering that Cry1-resistant FAW have truncated ABCC2 proteins, it is possible that resistance could be overcome by targeting Cry1F to bind the remaining ABCC2 in resistant FAW. The longest predicted extracellular loop in ABCC1 was 25 aa long and could be used for nanobody production. The sequence of FAW ABCC1 is provided in SEQ ID NOS. 43, and the extracellular loop is provided in SEQ ID NOS. 44.

### Fifth and Sixth Identified Proteins

Additional candidate targets selected based on their topology included a peptidase with a single transmembrane domain followed by a 783 aa long extracellular C terminus (venom dipeptidyl peptidase-4-like isoform X1) SEQ ID NOS. 37 (extracellular domain antigen) and 38 (full-length) and a peptide transporter with transmembrane domains and a 205 aa long extracellular loop (peptide transporter family 1 isoform X1) SEQ ID NOS. 39 (extracellular domain antigen) and 40 (full-length).

### Example 12. Cry1F Toxin Core as Antigen for Nanobody Production

The Cry1F toxin is one of the most active Bacillus thuringiensis insecticidal proteins against FAW larvae and is produced in transgenic corn as a FAW trait. Initial efforts focused on using protruding loops in domain II of the toxin which determine Cry toxin binding specificity (Dean, et al. 1996; Jurat-Fuentes and Adang, 2001), or the whole domain II as antigens (SEQ ID. NOS. 34). The whole Cry1F toxin core as obtained by trypsinization of the protoxin form was used in the following examples.

### Example 13. Expression and Purification of Receptor Antigens in Sf9 Insect Cells

Expression of the NAAT and Cadherin antigens in Spodoptera-derived Sf9 cells ensured these proteins carry any post-translational modifications similar to the native proteins. For cloning and expression of NAAT, both a GST fusion and His-tag epitope were cloned at the N-terminus of the antigen peptide for affinity purification (SEQ ID NOS. 48). For cloning and expression of cadherin, a His-tag epitope was cloned at the N-terminus for affinity purification (SEQ ID NOS. 49). A Precision Protease (Sigma-Aldrich) cleavage site (NAAT) or TEV (Sigma-Aldrich) cleavage site (cadherin) was cloned immediately upstream of the antigen sequence to enable subsequent purification of the antigen peptide free of any epitope tag.

The NAAT and cadherin antigen constructs were cloned into a baculovirus expression vector and transfected into Sf9 cells.

The fusion proteins were collected by binding to a His-Trap column (Cytiva) via the His-tag on the fusion protein. After elution from the His-Trap column, purified fusion proteins were cleaved overnight with PreScission or TEV Protease to remove the GST and His tags. While the GST fusion was successfully cleaved from NAAT, the His-epitope tag was only partially removed from the Cadherin antigen. NAAT and cadherin antigen peptides were purified over a Superdex 75 size or Superdex 200 (Cytiva) exclusion column, respectively. Peptide mass fingerprinting confirmed the identity of the purified peptides. For the cadherin antigen, aggregation of the peptide could not be avoided. However, when 1 mM EGTA was included in the buffer, aggregation was minimized.

### Example 14. Expression and Purification of Cry1Fa Core Toxin for Immunization

The full length Cry1F toxin (SEQ ID NOS. 34) was produced in a recombinant Bt HD-73 mutant strain harboring the pHT315 vector with the cry1F toxin gene under the cry1Ac promoter. For purification, parasporal crystals produced in Bt cultures were solubilized in buffer (50 mM Na₂CO₃, 0.1% β-mercaptoethanol, 0.1 M NaCl, pH 10.5) and solubilized Cry1F protoxin purified using anion exchange columns (HiTrap Q HP, GE Healthcare) connected to an AKTA FPLC (GE Healthcare). Protoxin was eluted with a gradient of 1 M NaCl in carbonate buffer (50 mM Na₂CO₃, 50 mM NaHCO₃ pH 9.8). A single major elution peak was detected and fractions in that peak were pooled, analyzed by SDS-10% PAGE and used in bioassays. Protoxin was activated using bovine trypsin and the activated toxin core was purified following the same anion exchange procedure as the full-length toxin.

### Example 15. Design, Production and Purification of a Novel Chimeric Cry Toxin Protein Used for Identification of cry1F Domain II-Specific Nanobodies

A toxin containing domain II of Cry1F and dissimilar domains I and III was needed to affinity-select nanobodies targeting domain II of Cry1F as the most plausible to affect toxin binding to new targets. Comparison of protein sequence identity among selected three-domain Cry toxins identified Cry2A toxins as displaying the lowest sequence identity in both domains 1 (24%) and III (16%) with the Cry1F toxin core. Consequently, we used comparisons of a Cry2Aa model (1i5pA in the Protein DataBank) with Cry1F and identified the different toxin domains to design a chimera containing domains I and III of Cry2Aa and domain II of Cry1F (2Aa/1F/2Aa), as provided in SEQ ID NOS. 50. A predicted model of this chimera toxin from Phyre 2 indicates folding similar to the three-domain folding in other Cry toxins.

### Example 16. Llama Immunization, V_{H}H Library Construction and Nanobody Screening

Llamas were subcutaneously injected on days 0, 7, 14, 21, 28 and 35, each time with about 100-160 μg of antigen. A different animal was used for injection of each of the three antigens: NAAT, Cadherin, and cry1F. The adjuvant used was Gerbu adjuvant P™ (Gerbu, Germany). On day 40, about 100 ml anticoagulated blood was collected from each llama for lymphocyte preparation.

V_{H}H antibody libraries were constructed from the llama lymphocytes to screen for the presence of antigen-specific Nanobodies (Nbs). To this end, total RNA from peripheral blood lymphocytes was used as template for first strand cDNA synthesis with an oligo(dT) primer. Using this cDNA, the V_{H}H encoding sequences were amplified by PCR, digested with SAPI, and cloned into the SAPI sites of the phagemid vector pMECS-GG.

The Nanobody gene cloned in pMECS phagemid vector (Vincke et al., 2012) contains PelB signal sequence at the N-terminus and HA tag and His6 tag at the C-terminus (PelB leader-Nanobody-HA-His6). The PelB leader sequence directs the Nanobody to the periplasmic space of the E. coli and the HA and His6 tags can be used for the purification and detection of Nanobody (e.g. in ELISA, Western Blot, etc.).

In pMECS vector, the His6 tag is followed by an amber stop codon (TAG) and this amber stop codon is followed by gene III of M13 phage. In suppressor E. coli strains (e.g. TG1), the amber stop codon is read as glutamine and therefore the Nanobody is expressed as fusion protein with protein III of the phage which allows the display of Nanobody on the phage coat for panning. In non-suppressor E. coli strains (e. g., WK6), the amber stop codon is read as stop codon and therefore the resulting Nanobody is not fused to protein III.

### Example 17. Isolation of Antigen-Specific Nanobodies

For identification of cry1F-specific nanobodies, V_{H}H libraries were panned on solid-phase coated Cry1Fa antigen (100 μg/ml in 100 mM NaHCO3 pH 8.2) for 3 rounds. Out of these 285 colonies, 180 colonies scored positive for Cry1Fa. Based on sequence data of the colonies positive on Cry1Fa, 132 different full length Nanobodies were distinguished, belonging to 53 different CDR3 groups (B-cell lineages). Some exemplary monospecific nanobody sequences are provided (Cry1F monospecific nanobody #5: SEQ ID NOS. 67 (DNA), SEQ ID NOS. 72 (protein); #7: SEQ ID NOS. 69 (DNA), SEQ ID NOS. 70 (protein); #51: SEQ ID NOS. 71 (DNA), SEQ ID NOS. 72 (protein)).

For identification of cadherin-specific nanobodies, the V_{H}H library was panned on solid-phase coated tagless FAW Cadherin antigen (a batch different from the one used for immunization, at 100 μg/ml in 100 mM NaHCO₃ pH 8.2) for 3 rounds. Out of 380 colonies, 324 scored positive for Sp.f Cadherin. Based on sequence data of the colonies positive on Sp.f Cadherin, 121 different full-length Nanobodies were distinguished, belonging to 25 different CDR3 groups (B-cell lineages). Some exemplary selected monospecific nanobody sequences are provided (Cadherin monospecific nanobody #2: SEQ ID NOS. 85 (DNA), SEQ ID NOS. 86 (protein); #43: SEQ ID NOS. 87 (DNA), SEQ ID NOS. 88 (protein); #46: SEQ ID NOS. 89 (DNA), SEQ ID NOS. 90 (protein); #48: SEQ ID NOS. 91 (DNA), SEQ ID NOS. 92 (protein); #50: SEQ ID NOS. 93 (DNA), SEQ ID NOS. 94 (protein).

For identification of NAAT-specific nanobodies, the V_{H}H library was panned, for 4 rounds, on biotinylated 57NAAT1 like immobilized (at 100 μg/ml in PBS) on streptavidin coated plates. The antigen used for panning carried an AviTag™ (Avidity, LLC) at N-terminus and had been biotinylated in vitro by the supplier at this tag using E. coli BirA enzyme. The enrichment for antigen-specific phages was assessed after each round of panning. Based on sequence data of the positive colonies, 4 different full length Nanobodies were distinguished, belonging to 4 different CDR3 groups (B-cell lineages).

The NAAT-specific V_{H}H library was panned and screened on non-biotinylated 57NAAT1 like coated directly (passively) to a well. In total, 380 colonies (95 from round 2, 190 from round 3 and 95 from round 4) were randomly selected and analyzed by ELISA for the presence of antigen-specific nanobodies in their periplasmic extracts. Out of these 380 colonies, 224 colonies scored positive for the target antigen (57NAAT1 like). Based on sequence data of the positive colonies, 30 different full length Nanobodies were distinguished, belonging to 24 different CDR3 groups (B-cell lineages). Some exemplary selected monospecific nanobody sequences are provided (NAAT monospecific nanobody #1: SEQ ID NOS. 73 (DNA), SEQ ID NOS. 74 (protein); #2: SEQ ID NOS. 75 (DNA), SEQ ID NOS. 76 (protein); #5: SEQ ID NOS. 77 (DNA), SEQ ID NOS. 78 (protein); #6: SEQ ID NOS. 79 (DNA), SEQ ID NOS. 80 (protein); #10: SEQ ID NOS. 81 (DNA), SEQ ID NOS. 82 (protein); #29: SEQ ID NOS. 83 (DNA), SEQ ID NOS. 84 (protein).

Nanobodies belonging to the same CDR3 group (same B-cell lineage) are very similar and their amino acid sequences suggest that they are from clonally-related B-cells resulting from somatic hypermutation or from the same B-cell but diversified due to RT and/or PCR error during library construction. Nanobodies belonging to the same CDR3 group recognize the same epitope but their other characteristics (e.g. affinity, potency, stability, expression yield, etc.) can be different.

### Example 18. Identification of Preferred cry1F Domain II-Specific Nanobodies

ELISA-based binding assays were performed using biotin-labeled 2Aa/1F/2Aa chimera and nanobodies developed against the Cry1F toxin core (termed Chi nanobodies). Microtiter ELISA plates (Immulon® 2HB 96 well plates, from Thermo Scientific) were coated overnight at room temperature with same amounts of individual Chi proteins in a total volume of 100 μl per well in TSE buffer (0.2 M Tris pH 8, 0.5 M sucrose, 1 mM EDTA). The wells were blocked in 0.5% BSA in PBS buffer (150 μl/well) and then were washed 3 times with binding buffer (0.1% BSA in PBS buffer, pH 7.4). Biotinylated 2Aa/1F/2Aa protein (0.1 μg in 100 μl of binding buffer) was added to each well, and reactions processed for one hour at room temperature with mild agitation. The solutions in each well were discarded and then the wells were washed 3 times with binding buffer for 10 min each. The plates were then incubated with streptavidin conjugated to horseradish peroxidase (1:5,000 dilutions in 100 μl binding buffer) for one hour at room temperature with shaking and then washed as above. Following the final wash, the wells were incubated with 1-step ultra TMB-ELISA substrate (50 μl/well) for 10 min. The reactions were stopped by adding 50 μl of 2 M H₂SO₄ and absorbance was measured at 450 nm using a microplate reader (Synergy™ HT from BioTek). Standard curves of two different preparations of biotinylated protein were performed and used for calculation of ng biotinylated protein bound per well. Four biological experiments were performed, each in technical duplicates. The data were analyzed using two-way ANOVA to identify monospecific nanobodies recognizing the 2Aa/1F/2Aa chimera as a proxy for binding to domain II of Cry1F.

Chi nanobodies were further characterized by determining the extent to which each individual nanobody protein can prevent the binding of cry1F toxin to the FAW BBMV. Microtiter ELISA plates (Immulon 2HB 96 well plates, Thermo Scientific) were coated overnight at room temperature with solubilized Sf BBMV proteins (1.6 μg/well) in a total volume of 100 μl per well of PBS buffer. Two different BBMV preparations were used.

Equal amounts of cry1F nanobodies were mixed with biotinylated Cry1F trypsin activated toxin (0.25 μg/one reaction) in binding buffer. Enough mixtures were made for 7 reactions per nanobody (one reaction volume is 100 μl) in 1.5 ml tubes and the tubes were mixed and incubated at −20° C. for later use.

The ELISA plate wells were blocked in 0.5% BSA in PBS buffer (150 μl/well) for one hour at room temperature and then washed 3 times with binding buffer (0.1% BSA in PBS buffer, pH 7.4). Binding assays were performed by adding the biotinylated Cry1F/nanobody mixes to wells coated with FAW, BBMV proteins, testing each mixture in triplicate wells, and the reactions processed for one hour at room temperature with mild agitation. The reactions in each well were then discarded and each well was washed 3 times with binding buffer for 10 min each. The plates were then incubated with streptavidin conjugated to horseradish peroxidase (1:5,000 dilutions in 100 μl binding buffer) for one hour at room temperature with shaking and then washed as above. Following the final wash, the wells were incubated with 1-step ultra TMB-ELISA substrate for 6 min. The reactions were stopped by adding 2 M H₂SO₄ and absorbance was measured at 450 nm using a microplate reader (BioTek Synergy HT). Standard curves to know the amount of biotinylated Cry1F bound to each well were performed. The data are the means from experiments with two different BBMV preparations, each performed in triplicate. The data were analyzed using One Way ANOVA (p=0.05). The data show that Domain II-specific nanobodies prevent the majority of cry1F toxin from binding to the BBMV. This result confirmed that cry1F Domain II is required for binding to the native receptor on the BBMV and that the cry1F nanobodies prevent that binding from occurring.

### Determining the Binding Affinity of Cry1F Domain II-Specific Nanobodies

Binding saturation assays using ELISA of biotinylated Cry1F trypsin activated protein were performed with Cry1F nanobodies previously selected based on their binding to a chimera protein containing domain II of Cry1F (chimera nanobodies). Two different preparations of Cry1F protein were used in this experiment. Microtiter ELISA plates (Immulon 2HB 96 well plates, Thermo Scientific) were coated overnight at room temperature with the same amounts of chimera nanobodies in a total volume of 100 μl per well of TSE buffer. The wells were then blocked in 0.5% BSA in PBS buffer (150 μl/well), followed by washing three times with binding buffer (0.1% BSA in PBS buffer, pH 7.4). Saturation binding assays were performed using increasing concentrations (from 0 to 100 nM) of biotinylated Cry1F protein as ligand. The total reaction volume was 100 μl in binding buffer, and reactions processed for one hour at room temperature with mild agitation. Non-specific binding was determined in separate reactions including 300-fold excess of the homologous unlabeled protein. The reactions in each well were discarded and each well was washed 3 times with binding buffer for 10 min each. The plates were then incubated with streptavidin conjugated to horseradish peroxidase (1:5,000 dilutions in 100 μl binding buffer) for one hour at room temperature with shaking and then washed as above. Following the final wash, the wells were incubated with 1-step ultra TMB-ELISA substrate for 10 min. The reactions were stopped by adding 2 M H₂SO₄ and absorbance was measured at 450 nm using a microplate reader (BioTek Synergy HT). Standard curves to know the amount of biotinylated protein represented by a specific A450 were performed for Cry1F biotinylated protein and used to calculate the total and nonspecific binding as ng of biotinylated protein bound per well. Specific binding of each labeled protein was calculated by subtracting non-specific from total binding. The data are the means of experiments performed with two Cry1F preparations, each tested in duplicate. The specific binding data were plotted and analyzed using the SigmaPlot v.11.2 software (Systat Software, San Jose, CA) to obtain the apparent dissociation constant (Kd) and concentration of binding sites (Bₘₐₓ). The model used is based on the existence of a single binding site. The sequences of cry1F monospecific nanobodies selected from this assay are provided (monospecific nanobody #5: SEQ ID NOS. 67 (DNA), SEQ ID NOS. 72 (protein); #7: SEQ ID NOS. 69 (DNA), SEQ ID NOS. 70 (protein); #51: SEQ ID NOS. 71 (DNA), SEQ ID NOS. 72 (protein).

### Example 19. Confirmation of Binding of NAAT and Cadherin Nanobodies to FAW BBMV

ELISA-based binding assays were performed using biotin-labeled solubilized Spodoptera frugiperda brush border membrane vesicles (Sf.BBMV) and NAAT nanobodies.

Microtiter ELISA plates (Immulon 2HB 96 well plates, Thermo Scientific) were coated overnight at room temperature with same amounts of NAAT or cadherin proteins in a total volume of 100 μl per well in TSE buffer (0.2 M Tris pH 8, 0.5 M sucrose, 1 mM EDTA). The wells were blocked in 0.5% BSA in PBS buffer (150 μl/well) and then were washed 3 times with binding buffer (0.1% BSA in PBS buffer, pH 7.4). Three different concentrations (0.1 μg, 1:3 and 1:10 dilutions) of biotinylated SfBBMV proteins were examined. The total reaction volume was 100 μl in binding buffer, and reactions processed for one hour at room temperature with mild agitation. The reactions in each well were discarded and then the wells were washed 3 times with binding buffer for 10 min each. The plates were then incubated with streptavidin conjugated to horseradish peroxidase (1:5,000 dilutions in 100 μl binding buffer) for one hour at room temperature with shaking and then washed as above. Following the final wash, the wells were incubated with 1-step ultra TMB-ELISA substrate (50 μl/well) for 10 min. The reactions were stopped by adding 50 μl of 2 M H₂SO₄ and absorbance was measured at 450 nm using a microplate reader (BioTek Synergy HT). Standard curves of two different preparations of biotinylated SfBBMV proteins were performed and used for calculation of ng biotinylated protein bound per well. The results are the means of 2 different experiments performed in duplicate (2 biological replicates). The data were analyzed using two-way ANOVA.

The sequences of NAAT monospecific nanobodies chosen for further analysis are provided SEQ ID NOS.: 73, 75, 77, 79, 81, 83, 129, 131, 133 (DNA) and 74, 76, 78, 80, 82, 84, 130, 132, 134 (Protein). The sequences of the various Cadherin monospecific nanobodies chosen for further analysis are provided: SEQ ID NOS. 85, 87, 89, 91, 93, 117, 119, 121, 123, 125, 127 (DNA) and 86, 88, 90, 92, 94, 118, 120, 122, 124, 126, 128 (Protein).

### Example 20. Cloning of Bispecific Nanobodies

Bispecific nanobodies were cloned into either the pMECS phagemid vector or the pHEN6c plasmid vector (Conrath et al., 2001). pHEN6 vector carries the PelB signal sequence at the N-terminus and HA epitope tag at the C-terminus and does not carry the gene III of M13 phage. In all cases, bispecific nanobodies are cloned in frame and immediately downstream of the PelB signal sequence (SEQ ID NOS. 66) and consist of a cry1F-specific nanobody (#5, 7 or 51 from Example 19) followed by a short peptide linker, a NAAT- or Cadherin-specific nanobody, and HA-His6 (pMECS) or HA epitope tags (pHEN6). See Table 4 for the combinations of bispecific nanobodies made and tested.

The peptide linkers cloned between the monospecific cry1F and NAAT or Cadherin nanobodies were chosen to optimize the antigen-binding properties and stability of the expressed fusions proteins. To this end, linkers with several different properties, as described below, were tested. See sequences in SEQ ID NOS. 54, 56, 58, 60, 62, 64 (protein) or SEQ ID NOS. 55, 57, 59, 61, 63, 65.

### “Rigid” Linkers:

PTPTn (Proline-Threonine, SEQ ID NOS. 64 (protein), 65 (DNA))—Proline and threonine amino acids are preferred amino acids found in natural linkers. Proline is a unique amino acid with a cyclic side chain that causes a very restricted conformation. Further, the lack of amide hydrogen on proline may prevent the formation of hydrogen bonds with other amino acids, thereby reducing the potential interaction of the linker with the other protein domains. On the other hand, threonine is a small polar amino acid that may help maintain the stability of the linker structure in the aqueous environment through formation of hydrogen bonds with water (reviewed in Chen et al., 2013).

AEAAAK3 (SEQ ID. NOS. 56 (protein), 57 (DNA)) was chosen as a variation of the natural linker between the lipoyl and E3 binding domains in pyruvate dehydrogenase enzyme and has been used in several fusion proteins, including to tobacco mosaic virus coat protein for overexpression of fusions proteins in tobacco or as a helical linker in transferrin-based fusion proteins in human cells.

Linker 218 as provided in SEQ ID NOS. 54 (protein), 55 (DNA) imparts enhanced proteolytic stability and reduced aggregation characteristics and was used in some exemplary embodiments.

### “Flexible” Linkers:

Gly8 (SEQ ID NOS. 62 (protein), 63 (DNA) and Gly4Ser1X3 (SEQ ID NOS. 60 (protein), 61 (DNA) linkers can increase the accessibility of an epitope to antibodies and improve protein folding. These linkers have also been demonstrated to be stable against proteolytic enzymes, especially important for stability of the fusion protein in the insect gut.

The ESGSVSSEQLAQFRSLD (SEQ ID NOS 58 (protein), 59 (DNA) linker has been used for the construction of a bioactive single-chain Fv antibody.

In total, more than 140 combinations of bispecific nanobodies were cloned and tested as shown in Table 4. The amino acid sequences of some exemplary bispecific nanobodies are provided in SEQ. ID. NOS. 96 (#22), 98 (#43), 100 (#48), 102 (#49), 104 (#50), 106 (#53), 108 (#62), 110 (#64), 112 (#76), 114 (#85) and 116 (#87) and the nucleotide sequence are SEQ. ID. NOS. 95 (#22), 97 (#43), 99 (#48), 101 (#49), 103 (#50), 105 (#53), 107 (#62), 109 (#64), 111 (#76), 113 (#85) and 115 (#87).

TABLE 4
Listing of bispecific nanobody clone combinations that were synthesized
and tested in bioassays. Sequences of some exemplary bispecific nanobodies are
provided in the list of sequences and the sequence listing.

| Bispecific | | | |
|---|---|---|---|
| Nanobody | | | NAAT or Cadherin |
| clone # | cry1F nanobody | Linker | nanobody |
| 1 | cry1F#5 (SEQ ID NO. | 218 (SEQ ID NO. 54 | NAAT29 (SEQ ID NO. |
| | 67 (DNA), 68 (protein)) | (protein), 55 (DNA)) | 83 (DNA), 84 (protein)) |
| 2 | cry1F#7 (SEQ ID NO. | 218 (SEQ ID NO. 54 | NAAT29 (SEQ ID NO. |
| | 69 (DNA), 70 (protein)) | (protein), 55 (DNA)) | 83 (DNA), 84 (protein)) |
| 3 | cry1F#5 (SEQ ID NO. | AEAAAK3 (SEQ ID | NAAT29 (SEQ ID NO. |
| | 67 (DNA), 68 (protein)) | NO. 56 (protein), | 83 (DNA), 84 (protein)) |
| | | 57(DNA)) | |
| 4 | cry1F#5 (SEQ ID NO. | ESGSV (SEQ ID NO. | NAAT29 (SEQ ID NO. |
| | 67 (DNA), 68 (protein)) | 58 (protein), 59 (DNA)) | 83 (DNA), 84 (protein)) |
| 5 | Cry1F#51 (SEQ ID | 218 (SEQ ID NO. 54 | NAAT29 (SEQ ID NO. |
| | NO. 71 (DNA), 72 | (protein), 55 (DNA)) | 83 (DNA), 84 (protein)) |
| | (protein)) | | |
| 6 | cry1F#5 (SEQ ID NO. | Gly4Ser1X3 (SEQ ID | NAAT29 (SEQ ID NO. |
| | 67 (DNA), 68 (protein)) | NO. 60 (protein), 61 | 83 (DNA), 84 (protein)) |
| | | (DNA)) | |
| 7 | cry1F#5 (SEQ ID NO. | Gly8 (SEQ ID NO. 62 | NAAT29 (SEQ ID NO. |
| | 67 (DNA), 68 (protein)) | (protein), 63 (DNA)) | 83 (DNA), 84 (protein)) |
| 8 | 67 (DNA), 68 (protein)) | (protein), 65 (DNA)) | 83 (DNA), 84 (protein)) |
| | cry1F#5 (SEQ ID NO. | PTPT (SEQ ID NO. 64 | NAAT29 (SEQ ID NO. |
| 9 | cry1F#7 (SEQ ID NO. | AEAAAK3 (SEQ ID | NAAT29 (SEQ ID NO. |
| | 69 (DNA), 70 (protein)) | NO. 56 (protein), | 83 (DNA), 84 (protein)) |
| | | 57(DNA)) | |
| 10 | Cry1F#51 (SEQ ID | AEAAAK3 (SEQ ID | NAAT29 (SEQ ID NO. |
| | NO. 71 (DNA), 72 | NO. 56 (protein), | 83 (DNA), 84 (protein)) |
| | (protein)) | 57(DNA)) | |
| 11 | cry1F#5 (SEQ ID NO. | 218 (SEQ ID NO. 54 | NAAT29 (SEQ ID NO. |
| | 67 (DNA), 68 (protein)) | (protein), 55 (DNA)) | 83 (DNA), 84 (protein)) |
| 12 | cry1F#7 (SEQ ID NO. | ESGSV (SEQ ID NO. | 83 (DNA), 84 (protein)) |
| | 69 (DNA), 70 (protein)) | 58 (protein), 59 (DNA)) | NAAT29 (SEQ ID NO. |
| 13 | Cry1F#51 (SEQ ID | ESGSV (SEQ ID NO. | NAAT29 (SEQ ID NO. |
| | NO. 71 (DNA), 72 | 58 (protein), 59 (DNA)) | 83 (DNA), 84 (protein)) |
| | (protein)) | | |
| 14 | cry1F#5 (SEQ ID NO. | AEAAAK3 (SEQ ID | Cad31(SEQ ID NO. |
| | 67 (DNA), 68 (protein)) | NO. 56 (protein), | 125 (DNA), 126 |
| | | 57(DNA)) | (protein)) |
| 15 | cry1F#7 (SEQ ID NO. | Gly4Ser1X3 (SEQ ID | NAAT29 (SEQ ID NO. |
| | 69 (DNA), 70 (protein)) | NO. 60 (protein), 61 | 83 (DNA), 84 (protein)) |
| | | (DNA)) | |
| 16 | Cry1F#51 (SEQ ID | Gly4Ser1X3 (SEQ ID | NAAT29 (SEQ ID NO. |
| | NO. 71 (DNA), 72 | NO. 60 (protein), 61 | 83 (DNA), 84 (protein)) |
| | (protein)) | (DNA)) | |
| 17 | cry1F#5 (SEQ ID NO. | 58 (protein), 59 (DNA)) | Cad31(SEQ ID NO. |
| | 67 (DNA), 68 (protein)) | ESGSV (SEQ ID NO. | 125 (DNA), 126 |
| | | | (protein)) |
| 18 | cry1F#7 (SEQ ID NO. | Gly8 (SEQ ID NO. 62 | NAAT29 (SEQ ID NO. |
| | 69 (DNA), 70 (protein)) | (protein), 63 (DNA)) | 83 (DNA), 84 (protein)) |
| 19 | Cry1F#51 (SEQ ID | Gly8 (SEQ ID NO. 62 | NAAT29 (SEQ ID NO. |
| | NO. 71 (DNA), 72 | (protein), 63 (DNA)) | 83 (DNA), 84 (protein)) |
| | (protein)) | | |
| 20 | cry1F#5 (SEQ ID NO. | Gly4Ser1X3 (SEQ ID | Cad31 (SEQ ID NO. |
| | 67 (DNA), 68 (protein)) | NO. 60 (protein), 61 | 125 (DNA), 126 |
| | | (DNA)) | (protein)) |
| 21 | 69 (DNA), 70 (protein)) | (protein), 65 (DNA)) | 83 (DNA), 84 (protein)) |
| | cry1F#7 (SEQ ID NO. | PTPT (SEQ ID NO. 64 | NAAT29 (SEQ ID NO. |
| 22 | Cry1F#51 (SEQ ID | PTPT (SEQ ID NO. 64 | NAAT29 (SEQ ID NO. |
| | NO. 71 (DNA), 72 | (protein), 65 (DNA)) | 83 (DNA), 84 (protein)) |
| | (protein)) | | |
| 23 | cry1F#5 (SEQ ID NO. | Gly8 (SEQ ID NO. 62 | Cad31 (SEQ ID NO. |
| | 67 (DNA), 68 (protein)) | (protein), 63 (DNA)) | 125 (DNA), 126 |
| | | | (protein)) |
| 24 | cry1F#7 (SEQ ID NO. | 218 (SEQ ID NO. 54 | Cad31 (SEQ ID NO. |
| | 69 (DNA), 70 (protein)) | (protein), 55 (DNA)) | 125 (DNA), 126 |
| | | | (protein)) |
| 25 | Cry1F#51 (SEQ ID | 218 (SEQ ID NO. 54 | Cad31 (SEQ ID NO. |
| | NO. 71 (DNA), 72 | (protein), 55 (DNA)) | 125 (DNA), 126 |
| | (protein)) | | (protein)) |
| 26 | cry1F#5 (SEQ ID NO. | PTPT (SEQ ID NO. 64 | Cad31 (SEQ ID NO. |
| | 67 (DNA), 68 (protein)) | (protein), 65 (DNA)) | 125 (DNA), 126 |
| | | | (protein)) |
| 27 | cry1F#7 (SEQ ID NO. | AEAAAK3 (SEQ ID | Cad31 (SEQ ID NO. |
| | 69 (DNA), 70 (protein)) | NO. 56 (protein), | 125 (DNA), 126 |
| | | 57(DNA)) | (protein)) |
| 28 | Cry1F#51 (SEQ ID | AEAAAK3 (SEQ ID | Cad31 (SEQ ID NO. |
| | NO. 71 (DNA), 72 | NO. 56 (protein), | 125 (DNA), 126 |
| | (protein)) | 57(DNA)) | (protein)) |
| 30 | cry1F#7 (SEQ ID NO. | 58 (protein), 59 (DNA)) | Cad31 (SEQ ID NO. |
| | 69 (DNA), 70 (protein)) | ESGSV (SEQ ID NO. | 125 (DNA), 126 |
| | | | (protein)) |
| 31 | Cry1F#51 (SEQ ID | ESGSV (SEQ ID NO. | Cad31 (SEQ ID NO. |
| | NO. 71 (DNA), 72 | 58 (protein, 59 (DNA)) | 125 (DNA), 126 |
| | (protein)) | | (protein)) |
| 33 | cry1F#7 (SEQ ID NO. | Gly4Ser1X3 (SEQ ID | Cad31 (SEQ ID NO. |
| | 69 (DNA), 70 (protein)) | NO. 60 (protein), 61 | 125 (DNA), 126 |
| | | (DNA)) | (protein)) |
| 34 | Cry1F#51 (SEQ ID | Gly4Ser1X3 (SEQ ID | Cad31 (SEQ ID NO. |
| | NO. 71 (DNA), 72 | NO. 60 (protein), 61 | 125 (DNA), 126 |
| | (protein)) | (DNA)) | (protein)) |
| 36 | cry1F#7 (SEQ ID NO. | Gly8 (SEQ ID NO. 62 | Cad31 (SEQ ID NO. |
| | 69 (DNA), 70 (protein)) | (protein), 63 (DNA)) | 125 (DNA), 126 |
| | | | (protein)) |
| 37 | Cry1F#51 (SEQ ID | Gly8 (SEQ ID NO. 62 | Cad31 (SEQ ID NO. |
| | NO. 71 (DNA), 72 | (protein), 63 (DNA)) | 125 (DNA), 126 |
| | (protein)) | | (protein)) |
| 39 | cry1F#7 (SEQ ID NO. | PTPT (SEQ ID NO. 64 | Cad31 (SEQ ID NO. |
| | 69 (DNA), 70 (protein)) | (protein), 65 (DNA)) | 125 (DNA), 126 |
| | | | (protein)) |
| 40 | Cry1F#51 (SEQ ID | PTPT (SEQ ID NO. 64 | Cad31 (SEQ ID NO. |
| | NO. 71 (DNA), 72 | (protein, 65 (DNA)) | 125 (DNA), 126 |
| | (protein)) | | (protein)) |
| 41 | Cry1F#51 (SEQ ID | gly8 (SEQ ID NO. 62 | Cad2 (SEQ ID NO. 85 |
| | NO. 71 (DNA), 72 | (protein), 63 (DNA)) | (DNA), 86 (protein)) |
| | (protein)) | | |
| 42 | Cry1F#51 (SEQ ID | gly8 (SEQ ID NO. 62 | Cad50 (SEQ ID NO. |
| | NO. 71 (DNA), 72 | (protein), 63 (DNA)) | 93 (DNA), 94 (protein)) |
| | (protein)) | | |
| 43 | Cry1F#51 (SEQ ID | PTPT (SEQ ID NO. 64 | Cad48 (SEQ ID NO. |
| | NO. 71 (DNA), 72 | (protein), 65 (DNA)) | 91 (DNA), 92 (protein)) |
| | (protein)) | | |
| 44 | Cry1F#51 (SEQ ID | gly8 (SEQ ID NO. 62 | Cad38 (SEQ ID NO. |
| | NO. 71 (DNA), 72 | (protein), 63 (DNA)) | 121 (DNA), 122 |
| | (protein)) | | (protein)) |
| 45 | Cry1F#51 (SEQ ID | gly8 (SEQ ID NO. 62 | Cad51 (SEQ ID NO. |
| | NO. 71 (DNA), 72 | (protein), 63 (DNA)) | 119 (DNA), 120 |
| | (protein)) | | (protein)) |
| 46 | Cry1F#51 (SEQ ID | PTPT (SEQ ID NO. 64 | Cad49 (SEQ ID NO. |
| | NO. 71 (DNA), 72 | (protein), 65 (DNA)) | 117 (DNA), 118 |
| | (protein)) | | (protein)) |
| 47 | Cry1F#51 (SEQ ID | gly8 (SEQ ID NO. 62 | Cad41 (SEQ ID NO. |
| | NO. 71 (DNA), 72 | (protein), 63 (DNA)) | 127 (DNA), 128 |
| | (protein)) | | (protein)) |
| 48 | Cry1F#51 (SEQ ID | PTPT (SEQ ID NO. 64 | Cad2 (SEQ ID NO. 85 |
| | NO. 71 (DNA), 72 | (protein), 65 (DNA)) | (DNA), 86 (protein)) |
| | (protein)) | | |
| 49 | Cry1F#51 (SEQ ID | PTPT (SEQ ID NO. 64 | Cad50 (SEQ ID NO. |
| | NO. 71 (DNA), 72 | (protein), 65 (DNA)) | 93 (DNA), 94 (protein)) |
| | (protein)) | | |
| 50 | Cry1F#51 (SEQ ID | gly8 (SEQ ID NO. 62 | Cad43 (SEQ ID NO. |
| | NO. 71 (DNA), 72 | (protein), 63 (DNA)) | 87 (DNA), 88 (protein)) |
| | (protein)) | | |
| 51 | Cry1F#51 (SEQ ID | PTPT (SEQ ID NO. 64 | Cad38 (SEQ ID NO. |
| | NO. 71 (DNA), 72 | (protein), 65 (DNA)) | 121 (DNA), 122 |
| | (protein)) | | (protein)) |
| 52 | Cry1F#51 (SEQ ID | PTPT (SEQ ID NO. 64 | Cad51 (SEQ ID NO. |
| | NO. 71 (DNA), 72 | (protein), 65 (DNA)) | 119 (DNA), 120 |
| | (protein)) | | (protein)) |
| 53 | Cry1F#51 (SEQ ID | gly8 (SEQ ID NO. 62 | Cad46 (SEQ ID NO. |
| | NO. 71 (DNA), 72 | (protein), 63 (DNA)) | 89 (DNA), 90 (protein)) |
| | (protein)) | | |
| 54 | Cry1F#51 (SEQ ID | PTPT (SEQ ID NO. 64 | Cad41 (SEQ ID NO. |
| | NO. 71 (DNA), 72 | (protein), 65 (DNA)) | 127 (DNA), 128 |
| | (protein)) | | (protein)) |
| 55 | Cry1F#51 (SEQ ID | gly8 (SEQ ID NO. 62 | Cad47 (SEQ ID NO. |
| | NO. 71 (DNA), 72 | (protein), 63 (DNA)) | 123 (DNA), 124 |
| | (protein)) | | (protein)) |
| 56 | Cry1F#51 (SEQ ID | PTPT (SEQ ID NO. 64 | Cad43 (SEQ ID NO. |
| | NO. 71 (DNA), 72 | (protein), 65 (DNA)) | 87 (DNA), 88 (protein)) |
| | (protein)) | | |
| 57 | Cry1F#51 (SEQ ID | gly8 (SEQ ID NO. 62 | Cad31 (SEQ ID NO. |
| | NO. 71 (DNA), 72 | (protein), 63 (DNA)) | 125 (DNA), 126 |
| | (protein)) | | (protein)) |
| 58 | Cry1F#51 (SEQ ID | PTPT (SEQ ID NO. 64 | Cad46 (SEQ ID NO. |
| | NO. 71 (DNA), 72 | (protein), 65 (DNA)) | 89 (DNA), 90 (protein)) |
| | (protein)) | | |
| 59 | Cry1F#51 (SEQ ID | gly8 (SEQ ID NO. 62 | Cad49 (SEQ ID NO. |
| | NO. 71 (DNA), 72 | (protein), 63 (DNA)) | 117 (DNA), 118 |
| | (protein)) | | (protein)) |
| 60 | Cry1F#51 (SEQ ID | PTPT (SEQ ID NO. 64 | Cad47 (SEQ ID NO. |
| | NO. 71 (DNA), 72 | (protein), 65 (DNA)) | 123 (DNA), 124 |
| | (protein)) | | (protein)) |
| 61 | cry1F#7 (SEQ ID NO. | gly8 (SEQ ID NO. 62 | NAAT1 (SEQ ID NO. |
| | 69 (DNA), 70 (protein)) | (protein), 63 (DNA)) | 73 (DNA), 74 |
| | | | (PROTEIN)) |
| 62 | cry1F#7 (SEQ ID NO. | PTPT (SEQ ID NO. 64 | NAAT1 (SEQ ID NO. |
| | 69 (DNA), 70 (protein)) | (protein), 65 (DNA)) | 73 (DNA), 74 |
| | | | (PROTEIN)) |
| 63 | cry1F#7 (SEQ ID NO. | gly8 (SEQ ID NO. 62 | NAAT2 (SEQ ID NO. |
| | 69 (DNA), 70 (protein)) | (protein), 63 (DNA)) | 75 (DNA), 76 |
| | | | (PROTEIN)) |
| 64 | cry1F#7 (SEQ ID NO. | PTPT (SEQ ID NO. 64 | NAAT2 (SEQ ID NO. |
| | 69 (DNA), 70 (protein)) | (protein), 65 (DNA)) | 75 (DNA), 76 |
| | | | (PROTEIN)) |
| 65 | cry1F#7 (SEQ ID NO. | gly8 (SEQ ID NO. 62 | NAAT3 (SEQ ID NO. |
| | 69 (DNA), 70 (protein)) | (protein), 63 (DNA)) | 129 (DNA), 130 |
| | | | (PROTEIN)) |
| 66 | cry1F#7 (SEQ ID NO. | PTPT (SEQ ID NO. 64 | NAAT3 (SEQ ID NO. |
| | 69 (DNA), 70 (protein)) | (protein), 65 (DNA)) | 129 (DNA), 130 |
| | | | (PROTEIN)) |
| 67 | cry1F#7 (SEQ ID NO. | gly8 (SEQ ID NO. 62 | NAAT4 (SEQ ID NO. |
| | 69 (DNA), 70 (protein)) | (protein), 63 (DNA)) | 131 (DNA), 132 |
| | | | (PROTEIN)) |
| 68 | cry1F#7 (SEQ ID NO. | PTPT (SEQ ID NO. 64 | NAAT4 (SEQ ID NO. |
| | 69 (DNA), 70 (protein)) | (protein), 65 (DNA)) | 131 (DNA), 132 |
| | | | (PROTEIN)) |
| 69 | cry1F#7 (SEQ ID NO. | gly8 (SEQ ID NO. 62 | NAAT5 (SEQ ID NO. |
| | 69 (DNA), 70 (protein)) | (protein), 63 (DNA)) | 77 (DNA), 78 |
| | | | (PROTEIN)) |
| 70 | cry1F#7 (SEQ ID NO. | PTPT (SEQ ID NO. 64 | NAAT5 (SEQ ID NO. |
| | 69 (DNA), 70 (protein)) | (protein), 65 (DNA)) | 77 (DNA), 78 |
| | | | (PROTEIN)) |
| 71 | cry1F#7 (SEQ ID NO. | gly8 (SEQ ID NO. 62 | NAAT6 (SEQ ID NO. |
| | 69 (DNA), 70 (protein)) | (protein), 63 (DNA)) | 79 (DNA), 80 |
| | | | (PROTEIN)) |
| 72 | cry1F#7 (SEQ ID NO. | PTPT (SEQ ID NO. 64 | NAAT6 (SEQ ID NO. |
| | 69 (DNA), 70 (protein)) | (protein), 65 (DNA)) | 79 (DNA), 80 |
| | | | (PROTEIN)) |
| 73 | cry1F#7 (SEQ ID NO. | gly8 (SEQ ID NO. 62 | NAAT7 (SEQ ID NO. |
| | 69 (DNA), 70 (protein)) | (protein), 63 (DNA)) | 133 (DNA), 134 |
| | | | (PROTEIN)) |
| 74 | cry1F#7 (SEQ ID NO. | PTPT (SEQ ID NO. 64 | NAAT7 (SEQ ID NO. |
| | 69 (DNA), 70 (protein)) | (protein), 65 (DNA)) | 133 (DNA), 134 |
| | | | (PROTEIN)) |
| 75 | cry1F#7 (SEQ ID NO. | gly8 (SEQ ID NO. 62 | NAAT10 (SEQ ID NO. |
| | 69 (DNA), 70 (protein)) | (protein), 63 (DNA)) | 81 (DNA), 82 |
| | | | (PROTEIN)) |
| 76 | cry1F#7 (SEQ ID NO. | PTPT (SEQ ID NO. 64 | NAAT10 (SEQ ID NO. |
| | 69 (DNA), 70 (protein)) | (protein), 65 (DNA)) | 81 (DNA), 82 |
| | | | (PROTEIN)) |
| 77 | Cry1F#51 (SEQ ID | gly8 (SEQ ID NO. 62 | NAAT1 (SEQ ID NO. |
| | NO. 71 (DNA), 72 | (protein), 63 (DNA)) | 73 (DNA), 74 |
| | (protein)) | | (PROTEIN)) |
| 78 | Cry1F#51 (SEQ ID | PTPT (SEQ ID NO. 64 | NAAT1 (SEQ ID NO. |
| | NO. 71 (DNA), 72 | (protein), 65 (DNA)) | 73 (DNA), 74 |
| | (protein)) | | (PROTEIN)) |
| 79 | Cry1F#51 (SEQ ID | gly8 (SEQ ID NO. 62 | NAAT2 (SEQ ID NO. |
| | NO. 71 (DNA), 72 | (protein), 63 (DNA)) | 75 (DNA), 76 |
| | (protein)) | | (PROTEIN)) |
| 80 | Cry1F#51 (SEQ ID | PTPT (SEQ ID NO. 64 | NAAT2 (SEQ ID NO. |
| | NO. 71 (DNA), 72 | (protein), 65 (DNA)) | 75 (DNA), 76 |
| | (protein)) | | (PROTEIN)) |
| 81 | Cry1F#51 (SEQ ID | gly8 (SEQ ID NO. 62 | NAAT3 (SEQ ID NO. |
| | NO. 71 (DNA), 72 | (protein), 63 (DNA)) | 129 (DNA), 130 |
| | (protein)) | | (PROTEIN)) |
| 82 | Cry1F#51 (SEQ ID | PTPT (SEQ ID NO. 64 | NAAT3 (SEQ ID NO. |
| | NO. 71 (DNA), 72 | (protein), 65 (DNA)) | 129 (DNA), 130 |
| | (protein)) | | (PROTEIN)) |
| 83 | Cry1F#51 (SEQ ID | gly8 (SEQ ID NO. 62 | NAAT4 (SEQ ID NO. |
| | NO. 71 (DNA), 72 | (protein), 63 (DNA)) | 131 (DNA), 132 |
| | (protein)) | | (PROTEIN)) |
| 84 | Cry1F#51 (SEQ ID | PTPT (SEQ ID NO. 64 | NAAT4 (SEQ ID NO. |
| | NO. 71 (DNA), 72 | (protein), 65 (DNA)) | 131 (DNA), 132 |
| | (protein)) | | (PROTEIN)) |
| 85 | Cry1F#51 (SEQ ID | gly8 (SEQ ID NO. 62 | NAAT5 (SEQ ID NO. |
| | NO. 71 (DNA), 72 | (protein), 63 (DNA)) | 77 (DNA), 78 |
| | (protein)) | | (PROTEIN)) |
| 86 | Cry1F#51 (SEQ ID | PTPT (SEQ ID NO. 64 | NAAT5 (SEQ ID NO. |
| | NO. 71 (DNA), 72 | (protein), 65 (DNA)) | 77 (DNA), 78 |
| | (protein)) | | (PROTEIN)) |
| 87 | Cry1F#51 (SEQ ID | gly8 (SEQ ID NO. 62 | NAAT6 (SEQ ID NO. |
| | NO. 71 (DNA), 72 | (protein), 63 (DNA)) | 79 (DNA), 80 |
| | (protein)) | | (PROTEIN)) |
| 88 | Cry1F#51 (SEQ ID | PTPT (SEQ ID NO. 64 | NAAT6 (SEQ ID NO. |
| | NO. 71 (DNA), 72 | (protein), 65 (DNA)) | 79 (DNA), 80 |
| | (protein)) | | (PROTEIN)) |
| 89 | Cry1F#51 (SEQ ID | gly8 (SEQ ID NO. 62 | NAAT7 (SEQ ID NO. |
| | NO. 71 (DNA), 72 | (protein), 63 (DNA)) | 133 (DNA), 134 |
| | (protein)) | | (PROTEIN)) |
| 90 | Cry1F#51 (SEQ ID | PTPT (SEQ ID NO. 64 | NAAT7 (SEQ ID NO. |
| | NO. 71 (DNA), 72 | (protein), 65 (DNA)) | 133 (DNA), 134 |
| | (protein)) | | (PROTEIN)) |
| 91 | Cry1F#51 (SEQ ID | gly8 (SEQ ID NO. 62 | NAAT10 (SEQ ID NO. |
| | NO. 71 (DNA), 72 | (protein), 63 (DNA)) | 81 (DNA), 82 |
| | (protein)) | | (PROTEIN)) |
| 92 | Cry1F#51 (SEQ ID | PTPT (SEQ ID NO. 64 | NAAT10 (SEQ ID NO. |
| | NO. 71 (DNA), 72 | (protein), 65 (DNA)) | 81 (DNA), 82 |
| | (protein)) | | (PROTEIN)) |
| 93 | cry1F#7 (SEQ ID NO. | gly8 (SEQ ID NO. 62 | Cad2 (SEQ ID NO. 85 |
| | 69 (DNA), 70 (protein)) | (protein), 63 (DNA)) | (DNA), 86 (protein)) |
| 94 | cry1F#7 (SEQ ID NO. | gly8 (SEQ ID NO. 62 | Cad50 (SEQ ID NO. |
| | 69 (DNA), 70 (protein)) | (protein, 63 (DNA)) | 93 (DNA), 94 (protein)) |
| 95 | cry1F#7 (SEQ ID NO. | PTPT (SEQ ID NO. 64 | Cad31 (SEQ ID NO. |
| | 69 (DNA), 70 (protein)) | (protein), 65 (DNA)) | 125 (DNA), 126 |
| | | | (protein)) |
| 96 | cry1F#7 (SEQ ID NO. | gly8 (SEQ ID NO. 62 | Cad38 (SEQ ID NO. |
| | 69 (DNA), 70 (protein)) | (protein), 63 (DNA)) | 121 (DNA), 122 |
| | | | (protein)) |
| 97 | cry1F#7 (SEQ ID NO. | gly8 (SEQ ID NO. 62 | Cad51 (SEQ ID NO. |
| | 69 (DNA), 70 (protein)) | (protein), 63 (DNA)) | 119 (DNA), 120 |
| | | | (protein)) |
| 98 | cry1F#7 (SEQ ID NO. | PTPT (SEQ ID NO. 64 | Cad49 (SEQ ID NO. |
| | 69 (DNA), 70 (protein)) | (protein), 65 (DNA)) | 117 (DNA), 118 |
| | | | (protein)) |
| 99 | cry1F#7 (SEQ ID NO. | gly8 (SEQ ID NO. 62 | Cad41 (SEQ ID NO. |
| | 69 (DNA), 70 (protein)) | (protein), 63 (DNA)) | 127 (DNA), 128 |
| | | | (protein)) |
| 100 | 69 (DNA), 70 (protein)) | (protein), 65 (DNA)) | (DNA), 86 (protein)) |
| | cry1F#7 (SEQ ID NO. | PTPT (SEQ ID NO. 64 | Cad2 (SEQ ID NO. 85 |
| 101 | cry1F#7 (SEQ ID NO. | PTPT (SEQ ID NO. 64 | Cad50 (SEQ ID NO. |
| | 69 (DNA), 70 (protein)) | (protein), 65 (DNA)) | 93 (DNA), 94 (protein)) |
| 102 | cry1F#7 (SEQ ID NO. | gly8 (SEQ ID NO. 62 | Cad43 (SEQ ID NO. |
| | 69 (DNA), 70 (protein)) | (protein), 63 (DNA)) | 87 (DNA), 88 (protein)) |
| 103 | cry1F#7 (SEQ ID NO. | PTPT (SEQ ID NO. 64 | Cad38 (SEQ ID NO. |
| | 69 (DNA), 70 (protein)) | (protein), 65 (DNA)) | 121 (DNA), 122 |
| | | | (protein)) |
| 104 | cry1F#7 (SEQ ID NO. | PTPT (SEQ ID NO. 64 | Cad51 (SEQ ID NO. |
| | 69 (DNA), 70 (protein)) | (protein), 65 (DNA)) | 119 (DNA), 120 |
| | | | (protein)) |
| 105 | cry1F#7 (SEQ ID NO. | gly8 (SEQ ID NO. 62 | Cad46 (SEQ ID NO. |
| | 69 (DNA), 70 (protein)) | (protein), 63 (DNA)) | 89 (DNA), 90 (protein)) |
| 106 | cry1F#7 (SEQ ID NO. | PTPT (SEQ ID NO. 64 | Cad41 (SEQ ID NO. |
| | 69 (DNA), 70 (protein)) | (protein), 65 (DNA)) | 127 (DNA), 128 |
| | | | (protein)) |
| 107 | cry1F#7 (SEQ ID NO. | gly8 (SEQ ID NO. 62 | Cad47 (SEQ ID NO. |
| | 69 (DNA), 70 (protein)) | (protein), 63 (DNA)) | 123 (DNA), 124 |
| | | | (protein)) |
| 108 | cry1F#7 (SEQ ID NO. | PTPT (SEQ ID NO. 64 | Cad43 (SEQ ID NO. |
| | 69 (DNA), 70 (protein)) | (protein), 65 (DNA)) | 87 (DNA), 88 (protein)) |
| 109 | cry1F#7 (SEQ ID NO. | gly8 (SEQ ID NO. 62 | Cad31 (SEQ ID NO. |
| | 69 (DNA), 70 (protein)) | (protein), 63 (DNA)) | 125 (DNA), 126 |
| | | | (protein)) |
| 110 | cry1F#7 (SEQ ID NO. | PTPT (SEQ ID NO. 64 | Cad46 (SEQ ID NO. |
| | 69 (DNA), 70 (protein)) | (protein), 65 (DNA)) | 89 (DNA), 90 (protein)) |
| 111 | cry1F#7 (SEQ ID NO. | gly8 (SEQ ID NO. 62 | Cad49 (SEQ ID NO. |
| | 69 (DNA), 70 (protein)) | (protein), 63 (DNA)) | 117 (DNA), 118 |
| | | | (protein)) |
| 112 | cry1F#7 (SEQ ID NO. | PTPT (SEQ ID NO. 64 | Cad47 (SEQ ID NO. |
| | 69 (DNA), 70 (protein)) | (protein), 65 (DNA)) | 123 (DNA), 124 |
| | | | (protein)) |
| 113 | cry1F#5 (SEQ ID NO. | gly8 (SEQ ID NO. 62 | NAAT1 (SEQ ID NO. |
| | 67 (DNA), 68 (protein)) | (protein), 63 (DNA)) | 73 (DNA), 74 |
| | | | (PROTEIN)) |
| 114 | cry1F#5 (SEQ ID NO. | PTPT (SEQ ID NO. 64 | NAAT2 (SEQ ID NO. |
| | 67 (DNA), 68 (protein)) | (protein), 65 (DNA)) | 75 (DNA), 76 |
| | | | (PROTEIN)) |
| 115 | cry1F#5 (SEQ ID NO. | gly8 (SEQ ID NO. 62 | NAAT3 (SEQ ID NO. |
| | 67 (DNA), 68 (protein)) | (protein), 63 (DNA)) | 129 (DNA), 130 |
| | | | (PROTEIN)) |
| 116 | cry1F#5 (SEQ ID NO. | PTPT (SEQ ID NO. 64 | NAAT3 (SEQ ID NO. |
| | 67 (DNA), 68 (protein)) | (protein), 65 (DNA)) | 129 (DNA), 130 |
| | | | (PROTEIN)) |
| 117 | cry1F#5 (SEQ ID NO. | gly8 (SEQ ID NO. 62 | NAAT4 (SEQ ID NO. |
| | 67 (DNA), 68 (protein)) | (protein), 63 (DNA)) | 131 (DNA), 132 |
| | | | (PROTEIN)) |
| 118 | cry1F#5 (SEQ ID NO. | PTPT (SEQ ID NO. 64 | NAAT4 (SEQ ID NO. |
| | 67 (DNA), 68 (protein)) | (protein), 65 (DNA)) | 131 (DNA), 132 |
| | | | (PROTEIN)) |
| 119 | cry1F#5 (SEQ ID NO. | gly8 (SEQ ID NO. 62 | NAAT5 (SEQ ID NO. |
| | 67 (DNA), 68 (protein)) | (protein), 63 (DNA)) | 77 (DNA), 78 |
| | | | (PROTEIN)) |
| 120 | cry1F#5 (SEQ ID NO. | PTPT (SEQ ID NO. 64 | NAAT5 (SEQ ID NO. |
| | 67 (DNA), 68 (protein)) | (protein), 65 (DNA)) | 77 (DNA), 78 |
| | | | (PROTEIN)) |
| 121 | cry1F#5 (SEQ ID NO. | gly8 (SEQ ID NO. 62 | NAAT6 (SEQ ID NO. |
| | 67 (DNA), 68 (protein)) | (protein), 63 (DNA)) | 79 (DNA), 80 |
| | | | (PROTEIN)) |
| 122 | cry1F#5 (SEQ ID NO. | PTPT (SEQ ID NO. 64 | NAAT6 (SEQ ID NO. |
| | 67 (DNA), 68 (protein)) | (protein), 65 (DNA)) | 79 (DNA), 80 |
| | | | (PROTEIN)) |
| 123 | cry1F#5 (SEQ ID NO. | gly8 (SEQ ID NO. 62 | NAAT7 (SEQ ID NO. |
| | 67 (DNA), 68 (protein)) | (protein), 63 (DNA)) | 133 (DNA), 134 |
| | | | (PROTEIN)) |
| 124 | cry1F#5 (SEQ ID NO. | PTPT (SEQ ID NO. 64 | NAAT7 (SEQ ID NO. |
| | 67 (DNA), 68 (protein)) | (protein), 65 (DNA)) | 133 (DNA), 134 |
| | | | (PROTEIN)) |
| 125 | cry1F#5 (SEQ ID NO. | gly8 (SEQ ID NO. 62 | NAAT10 (SEQ ID NO. |
| | 67 (DNA), 68 (protein)) | (protein), 63 (DNA)) | 81 (DNA), 82 |
| | | | (PROTEIN)) |
| 126 | cry1F#5 (SEQ ID NO. | PTPT (SEQ ID NO. 64 | NAAT10 (SEQ ID NO. |
| | 67 (DNA), 68 (protein)) | (protein), 65 (DNA)) | 81 (DNA), 82 |
| | | | (PROTEIN)) |
| 127 | cry1F#5 (SEQ ID NO. | PTPT (SEQ ID NO. 64 | NAAT1 (SEQ ID NO. |
| | 67 (DNA), 68 (protein)) | (protein), 65 (DNA)) | 73 (DNA), 74 |
| | | | (PROTEIN)) |
| 128 | cry1F#5 (SEQ ID NO. | gly8 (SEQ ID NO. 62 | Cad2 (SEQ ID NO. 85 |
| | 67 (DNA), 68 (protein)) | (protein), 63 (DNA)) | (DNA), 86 (protein)) |
| 129 | cry1F#5 (SEQ ID NO. | gly8 (SEQ ID NO. 62 | Cad50 (SEQ ID NO. |
| | 67 (DNA), 68 (protein)) | (protein), 63 (DNA)) | 93 (DNA), 94 (protein)) |
| 130 | cry1F#5 (SEQ ID NO. | PTPT (SEQ ID NO. 64 | Cad31 (SEQ ID NO. |
| | 67 (DNA), 68 (protein)) | (protein), 65 (DNA)) | 125 (DNA), 126 |
| | | | (protein)) |
| 131 | cry1F#5 (SEQ ID NO. | gly8 (SEQ ID NO. 62 | Cad38 (SEQ ID NO. |
| | 67 (DNA), 68 (protein)) | (protein), 63 (DNA)) | 121 (DNA), 122 |
| | | | (protein)) |
| 132 | cry1F#5 (SEQ ID NO. | gly8 (SEQ ID NO. 62 | Cad51 (SEQ ID NO. |
| | 67 (DNA), 68 (protein)) | (protein), 63 (DNA)) | 119 (DNA), 120 |
| | | | (protein)) |
| 133 | cry1F#5 (SEQ ID NO. | PTPT (SEQ ID NO. 64 | Cad49 (SEQ ID NO. |
| | 67 (DNA), 68 (protein)) | (protein), 65 (DNA)) | 117 (DNA), 118 |
| | | | (protein)) |
| 134 | cry1F#5 (SEQ ID NO. | gly8 (SEQ ID NO. 62 | Cad41 (SEQ ID NO. |
| | 67 (DNA), 68 (protein)) | (protein), 63 (DNA)) | 127 (DNA), 128 |
| | | | (protein)) |
| 135 | 67 (DNA), 68 (protein)) | (protein), 65 (DNA)) | (DNA), 86 (protein)) |
| | cry1F#5 (SEQ ID NO. | PTPT (SEQ ID NO. 64 | Cad2 (SEQ ID NO. 85 |
| 136 | cry1F#5 (SEQ ID NO. | PTPT (SEQ ID NO. 64 | Cad50 (SEQ ID NO. |
| | 67 (DNA), 68 (protein)) | (protein), 65 (DNA)) | 93 (DNA), 94 (protein)) |
| 137 | cry1F#5 (SEQ ID NO. | gly8 (SEQ ID NO. 62 | Cad43 (SEQ ID NO. |
| | 67 (DNA), 68 (protein)) | (protein, 63 (DNA)) | 87 (DNA), 88 (protein)) |
| 138 | cry1F#5 (SEQ ID NO. | PTPT (SEQ ID NO. 64 | Cad38 (SEQ ID NO. |
| | 67 (DNA), 68 (protein)) | (protein), 65 (DNA)) | 121 (DNA), 122 |
| | | | (protein)) |
| 139 | cry1F#5 (SEQ ID NO. | PTPT (SEQ ID NO. 64 | Cad51 (SEQ ID NO. |
| | 67 (DNA), 68 (protein)) | (protein), 65 (DNA)) | 119 (DNA), 120 |
| | | | (protein)) |
| 140 | cry1F#5 (SEQ ID NO. | gly8 (SEQ ID NO. 62 | Cad46 (SEQ ID NO. |
| | 67 (DNA), 68 (protein)) | (protein), 63 (DNA)) | 89 (DNA), 90 (protein)) |
| 141 | cry1F#5 (SEQ ID NO. | PTPT (SEQ ID NO. 64 | Cad41 (SEQ ID NO. |
| | 67 (DNA), 68 (protein)) | (protein), 65 (DNA)) | 127 (DNA), 128 |
| | | | (protein)) |
| 142 | cry1F#5 (SEQ ID NO. | gly8 (SEQ ID NO. 62 | Cad47 (SEQ ID NO. |
| | 67 (DNA), 68 (protein)) | (protein), 63 (DNA)) | 123 (DNA), 124 |
| | | | (protein)) |
| 143 | cry1F#5 (SEQ ID NO. | PTPT (SEQ ID NO. 64 | Cad43 (SEQ ID NO. |
| | 67 (DNA), 68 (protein)) | (protein), 65 (DNA)) | 87 (DNA), 88 (protein)) |
| 144 | cry1F#5 (SEQ ID NO. | gly8 (SEQ ID NO. 62 | Cad31 (SEQ ID NO. |
| | 67 (DNA), 68 (protein)) | (protein), 63 (DNA)) | 125 (DNA), 126 |
| | | | (protein)) |
| 145 | cry1F#5 (SEQ ID NO. | PTPT (SEQ ID NO. 64 | Cad46 (SEQ ID NO. |
| | 67 (DNA), 68 (protein)) | (protein), 65 (DNA)) | 89 (DNA), 90 (protein)) |
| 146 | cry1F#5 (SEQ ID NO. | gly8 (SEQ ID NO. 62 | Cad49 (SEQ ID NO. |
| | 67 (DNA), 68 (protein)) | (protein), 63 (DNA)) | 117 (DNA), 118 |
| | | | (protein)) |
| 147 | cry1F#5 (SEQ ID NO. | PTPT (SEQ ID NO. 64 | Cad47 (SEQ ID NO. |
| | 67 (DNA), 68 (protein)) | (protein), 65 (DNA)) | 123 (DNA), 124 |
| | | | (protein)) |
| 148 | cry1F#5 (SEQ ID NO. | gly8 (SEQ ID NO. 62 | NAAT2 (SEQ ID NO. |
| | 67 (DNA), 68 (protein)) | (protein), 63 (DNA)) | 75 (DNA), 76 |
| | | | (PROTEIN)) |

### Example 21. Purification of Bispecific Nanobodies from E. coli

The plasmids expressing bispecific nanobodies were chemically transformed into WK6 E. coli. A single colony from each transformation reaction was used. Periplasmic membrane protein extractions were performed in 10 ml of TSE buffer (0.2 M Tris, pH 8, 0.5 M Sucrose and 1 mM EDTA), overnight at 4° C. in end to end shaking to ensure maximum extraction, and the supernatants were extensively dialyzed against PBS pH 7.4 buffer overnight at 4° C. to get rid of EDTA that would interfere with the subsequent purification process.

His-tagged BsNbs were purified using His-Trap-Q-HP columns and 20 mM Tris pH 7.4, 0.3 M NaCl, 20 mM imidazole. Elution of His-tagged BsNbs was performed using 0.5 M imidazole over 10 column volumes. Fractions within the observed peaks were pooled, aliquoted and saved at −20° C. for later use.

### Example 22. Bispecific Nanobodies Enhance the Activity of cry1F Toxin Against FAW

Bioassays of Purified BsNb with or without Trypsin Activated Cry1Fa (LC₅₀ Dose)

Bioassays with purified BsNbs were performed with or without Cry1Fa toxin. The Cry1F toxin was used at the LC₅₀ concentration estimated above. For the bioassays, the BsNb purified proteins were mixed with Cry1Fa toxin in a 3:1 molar ratio (Nb:toxin) in a final volume of 750 μl of autoclaved MilliQ water. The molecular weight of BsNbs used for molarity calculations was ˜30 kDa, the amount of BsNb based on a 3:1 molar ratio is 6.33 μM (14.25 μg in 750 μl volume). Mixtures were incubated at room temperature for one hour and then stored on ice until applied on the surface of meridic diet. Once the meridic diet had set and dried in individual wells of 128-cell polystyrene bioassay trays, all treatments were applied on the diet surface, with 16 wells used per one treatment. The buffer used for BsNb extraction and water used for dilutions were used as control treatments. After the treatments had dried on the diet, a single FAW neonate was placed per well and trays incubated at 26±2° C. and 16L:8D photoperiod. Mortality was determined after 7 days of incubation.

The LC₅₀ and LC₉₀ of trypsin activated Cry1Fa were calculated by Probit analysis from two independent bioassays using the S. frugiperda strain from Benzon Inc. The buffer used for BsNb extraction and water used for dilutions were used as control treatments.

Based on bioassay data, numerous bispecific nanobodies enhance the activity of cry1F toxin significantly above its LC₅₀ mortality values over several repeated experiments. In several cases, 100% mortality of susceptible wild-type insects was observed. An example of bioassay data is shown in Table 5. Addition of Cry F toxin to the diet bioassay at the predetermined LC₅₀ dose results in 40.6% mortality of the wild-type insects, whereas buffer and water controls have only minimal effect on insects. Likewise, in the absence of cry1F toxin, minimal effects on insects from any of the bispecific nanobodies is observed. In contrast, several bispecific nanobodies (shaded) gave significantly higher insect mortality in this experiment (at least 75% of insects' dead). Furthermore, the average weight of surviving insects is significantly less than insects treated with cry1F toxin alone.

TABLE 5
Bioassay Data

| Wild-type | without Cry1Fa | | | with Cry1Fa (LC50 dose) | | |
|---|---|---|---|---|---|---|
| BsNb (see | | | % | | | % |
| Table 4) | Dead | Live | Mortality | Dead | Live | Mortality |
| Bs#45 | 0 | 16 | 0 | 12 | 4 | 75 |
| Bs#46 | 1 | 15 | 6.2 | 10 | 6 | 62.5 |
| Bs#47 | 1 | 15 | 6.2 | 10 | 6 | 62.5 |
| Bs#48 | 0 | 16 | 0 | 12 | 4 | 75 |
| Bs#49 | 2 | 14 | 12.5 | 12 | 4 | 75 |
| Bs#51 | 2 | 14 | 12.5 | 7 | 9 | 43.7 |
| Bs#55 | 0 | 16 | 0 | 7 | 9 | 43.7 |
| Bs#18 | 0 | 16 | 0 | 14 | 2 | 87.5 |
| Bs#33 | 0 | 16 | 0 | 16 | 0 | 100 |
| Cry1Fa | | | | 13 | 19 | 40.6 |
| (LC₅₀) | | | | | | |
| Buffer | 1 | 31 | 3.1 | | | |
| H2O | 1 | 31 | 3.1 | | | |

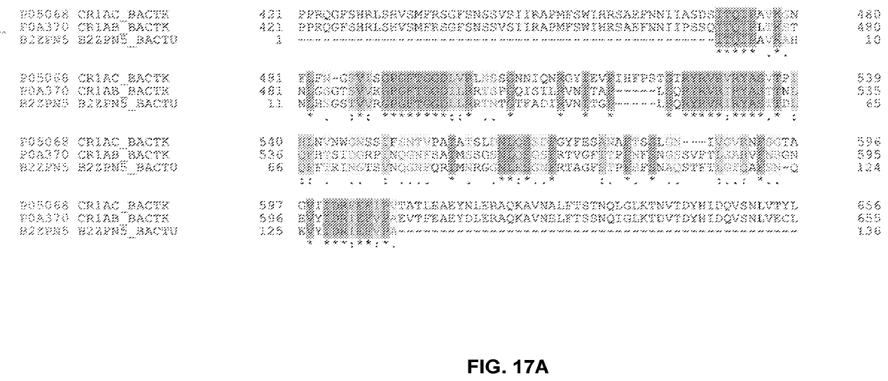

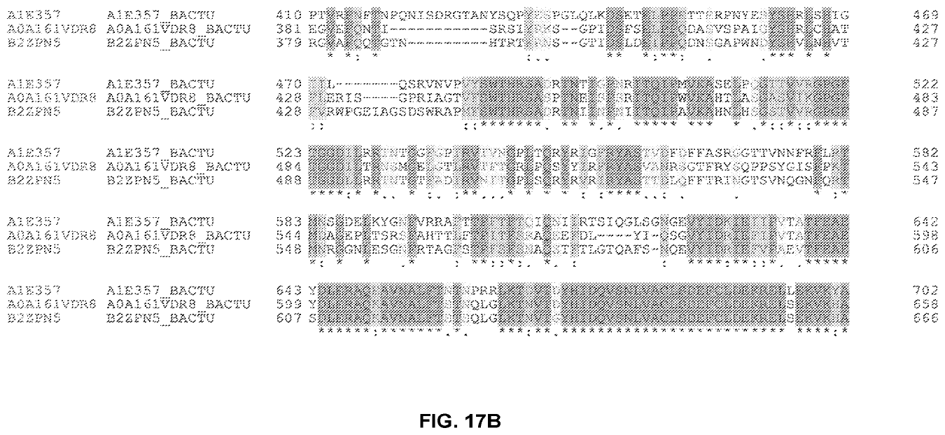

Cry1F-resistant insect line PR1 that lacks the functional cry1F toxin receptor, ABCC2, has been described (Banerjee et al., 2017). Treatment of cry1F toxin in bioassays using these insects, even at ˜20× higher dosage than the LC₅₀ in susceptible insects, has no significant effects on insect mortality. Table 3 shows treatment of PR1 insects with several bispecific nanobodies in the presence or absence of cry1F toxin. No effect of the bispecific nanobodies on PR1 insects in any case is seen (see Table 6). These results were repeated with most of the bispecific nanobodies, and no significant mortality was observed in any case. These results indicate that the ABCC2 receptor is apparently important for Cry1F toxicity in PR1 insects. However, since other toxins with different receptors, for instance Cry1Ab and Cry1Ac share sequence similarities with cry1F (FIG. 17A) and similarly Cry1B, Cry1 Da also exhibit sequence similarity with Cry1F (FIG. 17B), these BsNb may be used in conjunction with other toxins to target Cry1F-resistant insects. Furthermore, it may be possible to create a BsNb that binds to the known cry1F binding site in the ABCC2 receptor to restore cry1F insecticidal activity in resistant insects.

Table 6. Shows Effects of Treatment of PR1 Insects with Bispecific Nanobodies

At least 20 bispecific nanobodies, shown in Table 7 gave consistently higher mortality rates in susceptible insects than cry1F toxin alone over multiple experiments, 11 of these (shaded) were chosen for further analysis.

### Example 23. Binding of Cry1F Toxin to Bispecific Nanobody

To prove that the cry1F toxin binds to the bispecific nanobodies, the bispecific nanobody-cry1F complex was analyzed by size exclusion chromatography on an FPLC machine. Bispecific nanobodies were added at various molar ratios compared to cry1F toxin (3:1, 1:1, 0.5:1) and the presence of a complex versus free toxin or free bispecific nanobody was determined over collected fractions that elute from the size exclusion column. Proteins were run on acrylamide gel electrophoresis and stained by Coomassie Blue to visualize proteins.

When bispecific nanobody #64 (as an example) is incubated with cry1F at a 1:1 molar ratio, cry1F and the bispecific nanobody elute in the same fractions, indicating that they are present in a complex. In contrast, if the toxin is added in molar excess, both a complex and excess free toxin are observed, indicating that the excess toxin is not present in a complex. Likewise, when bispecific nanobody was added at 3:1 molar excess over cry1F toxin, both a complex and excess unbound bispecific nanobody were observed. These results show that the bispecific nanobody and cry1F are indeed bound in a complex and indicates that a 1:1 molar ratio is optimal for formation of that complex.

### Example 24. Binding of NAAT and Cadherin Nanobodies to Diamondback Moth BBMV

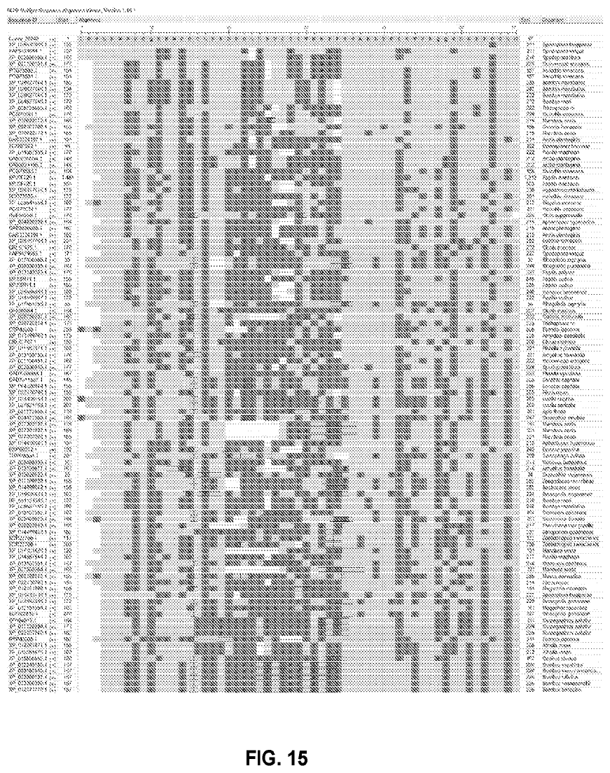

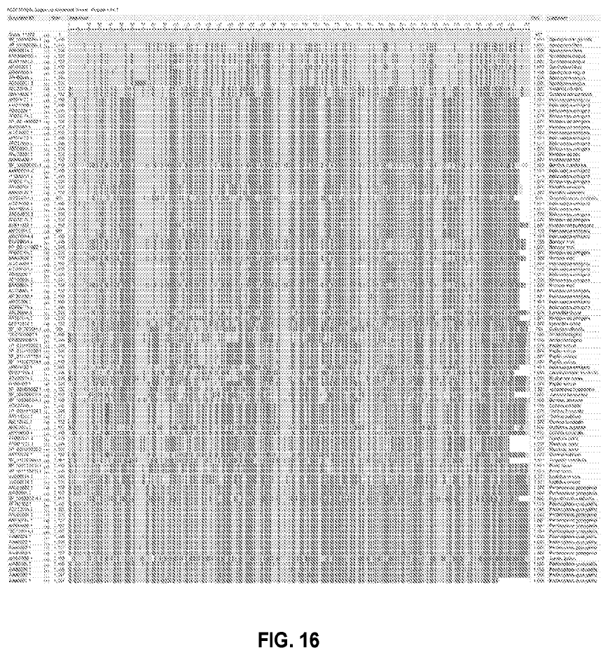

The utility of the nanobodies is significantly enhanced if they have activity against multiple insect pests. To determine if the NAAT and cadherin monospecific nanobodies might recognize the cognate receptor protein in other insect species, the 57 amino acid NAAT and 427 amino acid Cadherin antigen sequences were used in a Blast sequence homology search. Using Diamondback moth (DBM; Plutella xylostella) as an example, significant amino acid identity was found to be present (see FIGS. 15 and 16) suggesting that the existing nanobodies raised against FAW receptors may also recognize the homologous receptor proteins in Plutella and other species. Table 9 shows percent homology of NAAT and Cadherin target sequences to additional insects.

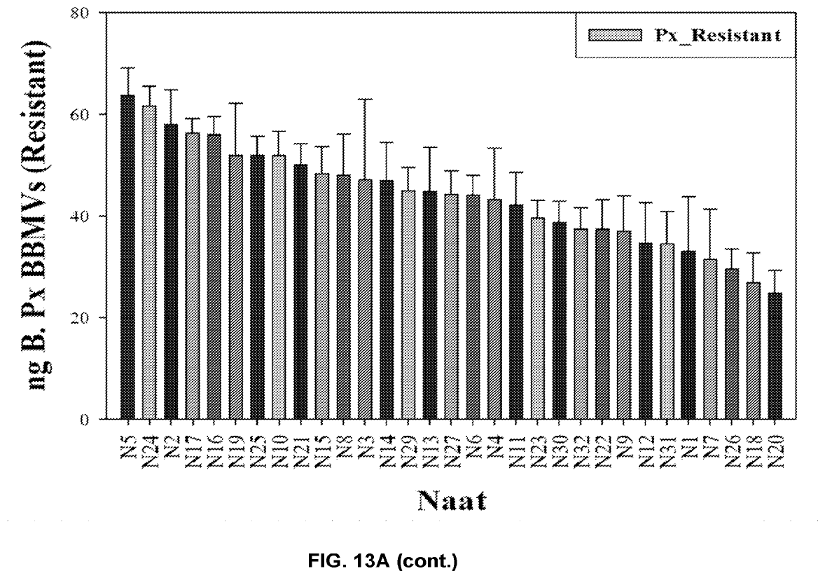

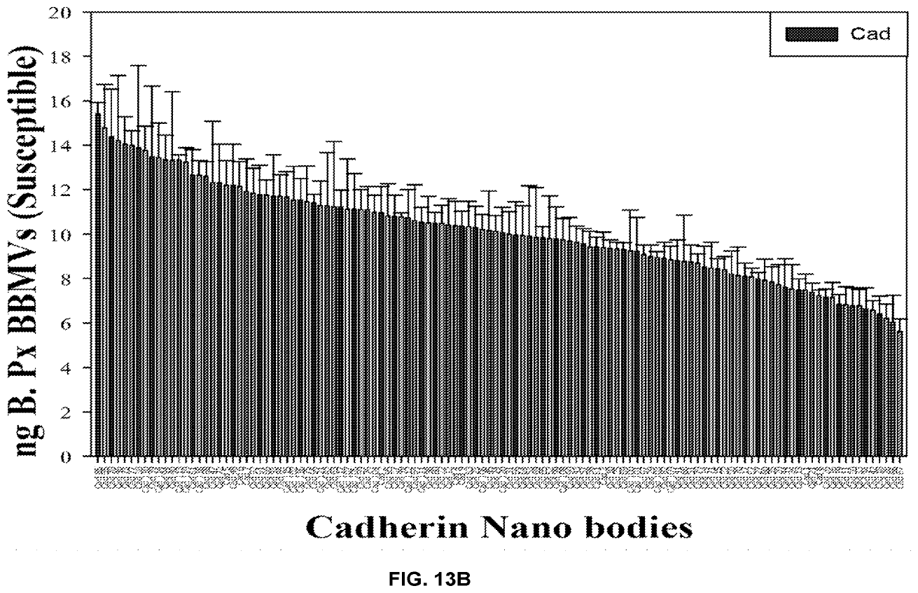

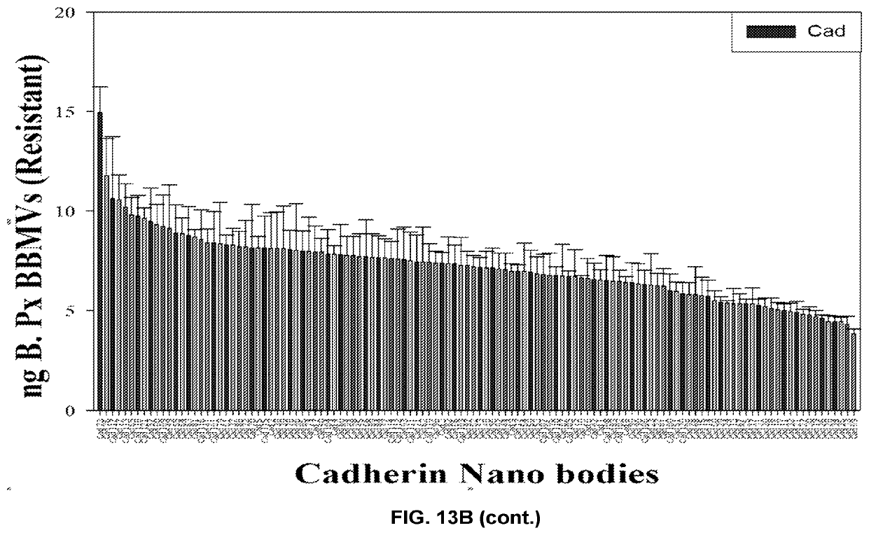

To determine if the existing NAAT and Cadherin nanobodies can recognize Diamondback moth BBMV, ELISA-based binding assays were performed using biotin-labeled solubilized Plutella brush border membrane vesicles and NAAT or cadherin nanobodies, using the same methodology as was used for FAW. BBMV derived from Plutella insects (Px.BBMV) that are susceptible to cry1F toxin as well as resistant to cry1F toxin were tested. FIGS. 13A and 13B provide results of these ELISA-based assays using NAAT or cadherin monospecific nanobodies respectively. The data given in FIGS. 13A and 13B identify NAAT and Cadherin nanobodies that recognize Plutella BBMV in both susceptible and resistant insects, indicating that they have utility in control of those insects.

### Example 25. Bioassay to Test Activity Across Plurality of Species and Toxins

Similar to recognizing new insects via their gut receptor homology to the antigens that created the bispecificic nanobodies, the utility of the nanobodies is also significantly enhanced if they recognize insect toxins with homology to the original Cry1F toxin antigen used to create the nanobodies, with examples of this shown in Table 10. Bioassays with purified bispecific nanobodies (BsNbs) were performed with results in Table 8 shows the study design to test the activity of each of the listed bispecific nanobodies with Cry1F or Cry1Ac or Cry1Ab protein toxins, against Diamondback Moth (DBM), Fall Armyworm (FAW) or Corn Earworm (CEW).

The protein toxins were used at LC₅₀. BsNb purified proteins were mixed with the indicated toxin (shown in Table 8) in a 3:1 molar ratio (BsNb:toxin) in a final volume of 75 μl of autoclaved MilliQ water. The molecular weight of BsNbs used for molarity calculations was ˜30 kDa, the amount of BsNb based on a 3:1 molar ratio is 6.33 μM (14.25 μg in 75 μl volume). Mixtures were incubated at room temperature for one hour and then stored on ice until applied on the surface of meridic diet. Once the meridic diet had set and dried in individual wells of 128-cell polystyrene bioassay trays, all treatments were applied on the diet surface, with 16 wells used per one treatment. The buffer used for BsNb extraction and water used for dilutions were used as control treatments. After the treatments had dried on the diet, a single DBM, FAW or CEW neonate was placed per well and trays incubated at 26±2° C. and 16L:8D photoperiod. Mortality was determined after 7 days of incubation.

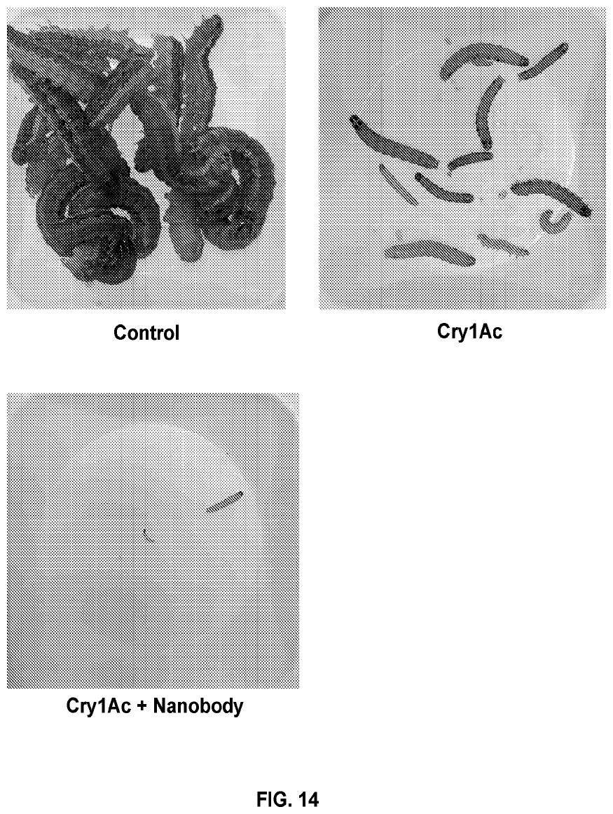

Table 8 shows the outcome of these studies and FIG. 14 shows photographs of one such study. As evident from the FIG. 14 and Table 8, Cry1Ac protein when used in conjunction with the bispecific nanobodies is very effective in killing and stunting FAW larvae. Further the combination is more effective than using Cry1Ac alone. Additionally, numerous bispecific nanobodies enhance the activity of Cry1F toxin significantly above its LC₅₀ mortality values across the three species tested. Interestingly, at least one of the BsNbs was also effective in stunting the growth of FAW when used with Cry1Ab. This is surprising since Cry1Ab alone is not effective against FAW. Additionally, multiple bispecific nanobodies generated activity of Cry1F against CEW, another protein not known to effectively control the insect.

TABLE 8
Bioassay Study Design and Outcome (“NT”-non tested, “()”-one
replicate, “+”-higher mortality rate over
control, “−”-no change over control, “+/−”-higher mortality
but the results were inconsistent or not statistically significant.

| BsNb | | | | | | |
|---|---|---|---|---|---|---|
| # | | | | | | |
| (see | | | | | | Cry1Ab |
| Table | | Cry1F | | | Cry1Ac | FAW |
| 4) | MsNb-Cry-link-target | DBM | FAW | CEW | FAW | stunting |
| 1 | cry1Nb5-218- | NT | − | − | − | − |
| | NAAT29 | | | | | |
| 4 | cry1Nb5-ESGSV- | (+) | +/− | NT | NT | NT |
| | NAAT29 | | | | | |
| 10 | cry1Nb51- | NT | + | − | − | − |
| | AEAAAK3- | | | | | |
| | NAAT29 | | | | | |
| 15 | cry1Nb7- | NT | +/− | − | + | − |
| | Gly4Ser1x3- | | | | | |
| | NAAT29 | | | | | |
| 18 | cry1Nb7-Gly8- | NT | + | − | + | − |
| | NAAT29 | | | | | |
| 21 | cry1Nb7-PTPT- | (+) | +/− | + | NT | NT |
| | NAAT29 | | | | | |
| 39 | cry1Nb7-PTPT- | NT | + | − | + | − |
| | NAAT31 | | | | | |
| 62 | cry1Nb7-PT-NAAT1 | NT | + | − | + | − |
| 64 | cry1Nb7-PT-NAAT2 | − | + | − | + | − |
| 76 | cry1Nb7-PT- | NT | + | +/− | + | + |
| | NAAT10 | | | | | |
| 100 | cry1Nb7-PT-Cad2 | (+) | + | + | NT | NT |
| 108 | cry1Nb7-PT-Cad43 | (−) | +/− | + | NT | NT |
| 111 | cry1Nb7-Gly8-Cad49 | (+) | + | +/− | NT | NT |
| 112 | cry1Nb7-PT-Cad47 | (+) | +/− | − | NT | NT |

Similarly, bispecific nanobodies with sequences directed to target FAW Cadherins and NAAT sequences were surprisingly also effective against a plurality of other insect pests including DBM and CEW when used with Cry1Ac. This further demonstrates that this approach can be used to target insect strains that are not susceptible to the native toxin. It is noted the selected membrane protein targets show a high degree of sequence conservation across these pests (FIGS. 15 and 16, and Table 9).

TABLE 9
Sequence conservation between membrane proteins (Cadherin and NAAT)
across multiple insect species

| | | | | | | | | |
|---|---|---|---|---|---|---|---|---|
| | Scientific | | | | | | | |
| | Name | Max | Total | Query | E | Per. | Acc. | |
| Description | (Insect) | Score | Score | Cover | value | ident | Len | Accession |

Cadherin

| | | | | | | | | |
|---|---|---|---|---|---|---|---|---|
| hypothetical protein | Spodoptera | 854 | 854 | 100% | 0 | 99.53 | 1658 | KAG8112339.1 |
| SFRUCORN_020876 | frugiperda | | | | | | | |
| [Spodoptera frugiperda] | | | | | | | | |
| protocadherin Fat 3-like | Spodoptera | 855 | 855 | 100% | 0 | 99.3 | 1734 | XP_035440763.1 |
| [Spodoptera frugiperda] | frugiperda | | | | | | | |
| hypothetical protein | Spodoptera | 852 | 852 | 100% | 0 | 99.3 | 1706 | KAF9820681.1 |
| SFRURICE_006703 | frugiperda | | | | | | | |
| [Spodoptera frugiperda] | | | | | | | | |
| truncated cadherin | Helicoverpa | 84.7 | 84.7 | 13% | 2.00E−16 | 66.67 | 771 | AVE17270.1 |
| [Helicoverpa punctigera] | punctigera | | | | | | | |
| truncated cadherin | Helicoverpa | 143 | 143 | 24% | 8.00E−36 | 66.35 | 1271 | AFB74167.1 |
| [Helicoverpa armigera] | armigera | | | | | | | |
| cadherin-like protein | Helicoverpa | 456 | 456 | 97% | 2.00E−145 | 55.13 | 1730 | AKH49609.1 |
| [Helicoverpa zea] | zea | | | | | | | |
| cadherin [Helicoverpa | Helicoverpa | 449 | 449 | 97% | 7.00E−143 | 55.11 | 1732 | AVE17268.1 |
| punctigera] | punctigera | | | | | | | |
| hypothetical protein | Heliothis | 355 | 672 | 97% | 3.00E−110 | 55.05 | 1304 | PCG67047.1 |
| B5V51_6905 [Heliothis | virescens | | | | | | | |
| virescens] | | | | | | | | |
| truncated cadherin | Helicoverpa | 302 | 302 | 64% | 2.00E−90 | 55.04 | 1441 | AWJ76613.1 |
| [Helicoverpa armigera] | armigera | | | | | | | |
| E-cadherin [Helicoverpa | Helicoverpa | 457 | 457 | 97% | 3.00E−146 | 54.89 | 1672 | AAU50668.1 |
| armigera] | armigera | | | | | | | |
| cadherin-like protein | Helicoverpa | 453 | 453 | 97% | 2.00E−144 | 54.89 | 1730 | AKH49605.1 |
| [Helicoverpa zea] | zea | | | | | | | |
| cadherin [Helicoverpa | Helicoverpa | 457 | 457 | 97% | 1.00E−145 | 54.65 | 1730 | AFB74170.1 |
| armigera] | armigera | | | | | | | |
| cadherin [Helicoverpa | Helicoverpa | 454 | 454 | 97% | 8.00E−145 | 54.42 | 1675 | AFQ60151.1 |
| armigera] | armigera | | | | | | | |
| cadherin-like protein | Ostrinia | 191 | 191 | 38% | 4.00E−58 | 54.17 | 175 | AGO01049.1 |
| [Ostrinia scapulalis] | scapulalis | | | | | | | |
| cadherin [Helicoverpa | Helicoverpa | 447 | 447 | 97% | 1.00E−142 | 53.94 | 1675 | AFQ60152.1 |
| armigera] | armigera | | | | | | | |
| cadherin-like protein | Helicoverpa | 446 | 446 | 97% | 1.00E−141 | 53.94 | 1730 | AAT67416.1 |
| [Helicoverpa armigera] | armigera | | | | | | | |
| cadherin-like Cry1Ac | Heliothis | 455 | 455 | 97% | 4.00E−145 | 53.92 | 1732 | AAV80768.1 |
| receptor [Heliothis | virescens | | | | | | | |
| virescens] | | | | | | | | |
| cadherin-like protein | Heliothis | 455 | 455 | 97% | 6.00E−145 | 53.68 | 1732 | AAK85198.1 |
| [Heliothis virescens] | virescens | | | | | | | |
| cadherin [Helicoverpa | Helicoverpa | 440 | 440 | 97% | 1.00E−139 | 53.46 | 1730 | AFB74168.1 |
| armigera] | armigera | | | | | | | |
| cadherin [Helicoverpa | Helicoverpa | 181 | 181 | 41% | 1.00E−48 | 51.67 | 1343 | AEC33256.1 |
| armigera] | armigera | | | | | | | |
| cadherin M1 [Ostrinia | Ostrinia | 412 | 412 | 97% | 1.00E−129 | 50.12 | 1716 | ACK37449.1 |
| nubilalis] | nubilalis | | | | | | | |
| protocadherin Fat 1-like | Ostrinia | 412 | 412 | 97% | 2.00E−129 | 50.12 | 1722 | XP_028161184.1 |
| [Ostrinia furnacalis] | furnacalis | | | | | | | |
| cadherin [Plutella | Plutella | 380 | 380 | 96% | 3.00E−119 | 49.64 | 1334 | ABI63545.1 |
| xylostella] | xylostella | | | | | | | |
| cadherin [Agrotis ipsilon] | Agrotis ipsilon | 388 | 388 | 96% | 2.00E−120 | 49.53 | 1760 | AEB97396.1 |
| cadherin [Ostrinia | Ostrinia | 73.2 | 73.2 | 20% | 6.00E−15 | 49.45 | 95 | AAT48603.1 |
| nubilalis] | nubilalis | | | | | | | |
| cadherin-like protein | Plutella | 379 | 379 | 96% | 2.00E−117 | 49.41 | 1716 | ABU41413.1 |
| [Plutella xylostella] | xylostella | | | | | | | |
| cadherin A2 [Ostrinia | Ostrinia | 230 | 230 | 56% | 1.00E−72 | 49.19 | 242 | ABB03903.1 |
| nubilalis] | nubilalis | | | | | | | |
| cadherin [Plutella | Plutella | 374 | 374 | 96% | 3.00E−117 | 49.17 | 1334 | ABI63546.1 |
| xylostella] | xylostella | | | | | | | |
| cadherin [Ostrinia | Ostrinia | 204 | 204 | 48% | 7.00E−63 | 48.82 | 218 | ADX42727.1 |
| nubilalis] | nubilalis | | | | | | | |
| unnamed protein | Plutella | 372 | 372 | 96% | 6.00E−117 | 48.57 | 1236 | CAG9135951.1 |
| product [Plutella | xylostella | | | | | | | |
| xylostella] | | | | | | | | |
| cadherin [Ostrinia | Ostrinia | 73.6 | 73.6 | 20% | 5.00E−15 | 48.35 | 95 | AAT48607.1 |
| nubilalis] | nubilalis | | | | | | | |
| cadherin [Ostrinia | Ostrinia | 203 | 203 | 48% | 3.00E−62 | 48.34 | 218 | ADX42726.1 |
| nubilalis] | nubilalis | | | | | | | |
| cadherin [Ostrinia | Ostrinia | 70.5 | 70.5 | 20% | 5.00E−14 | 47.25 | 95 | AAT48610.1 |
| nubilalis] | nubilalis | | | | | | | |
| cadherin [Ostrinia | Ostrinia | 166 | 166 | 39% | 1.00E−46 | 47.06 | 327 | ACK37451.1 |
| nubilalis] | nubilalis | | | | | | | |
| cadherin 1 [Diatraea | Diatraea | 378 | 378 | 98% | 4.00E−117 | 46.6 | 1718 | AFI81418.1 |
| saccharalis] | saccharalis | | | | | | | |
| cadherin [Ostrinia | Ostrinia | 68.2 | 68.2 | 20% | 4.00E−13 | 46.15 | 95 | AAT48604.1 |
| nubilalis] | nubilalis | | | | | | | |
| cadherin [Chrysodeixis | Chrysodeixis | 248 | 325 | 96% | 1.00E−71 | 44.92 | 1956 | QRU95334.1 |
| includens] | includens | | | | | | | |
| cadherin-like protein | Helicoverpa | 355 | 355 | 97% | 1.00E−108 | 43.15 | 1757 | AEE44121.1 |
| resistant allele r9 | armigera | | | | | | | |
| [Helicoverpa armigera] | | | | | | | | |
| cadherin-like protein | Helicoverpa | 355 | 355 | 97% | 4.00E−109 | 43.04 | 1756 | AEE44122.1 |
| susceptible isoform | armigera | | | | | | | |
| [Helicoverpa armigera] | | | | | | | | |
| unnamed protein | Plutella | 84.3 | 84.3 | 49% | 2.00E−16 | 29.28 | 451 | CAG9119464.1 |
| product [Plutella | xylostella | | | | | | | |
| xylostella] | | | | | | | | |
| hypothetical protein | Plutella | 100 | 100 | 72% | 2.00E−21 | 28.31 | 1404 | KAG7306076.1 |
| JYU34_008659 [Plutella | xylostella | | | | | | | |
| xylostella] | | | | | | | | |
| cadherin-23 [Plutella | Plutella | 95.1 | 136 | 72% | 1.00E−19 | 27.79 | 1890 | XP_037974876.1 |
| xylostella] | xylostella | | | | | | | |
| cadherin-23 [Diabrotica | Diabrotica | 111 | 111 | 86% | 6.00E−25 | 27.58 | 1888 | XP_028139379.1 |
| virgifera virgifera] | virgifera | | | | | | | |
| | virgifera | | | | | | | |
| cadherin-23 [Ostrinia | Ostrinia | 100 | 100 | 72% | 2.00E−21 | 27.58 | 2034 | XP_028165671.1 |
| furnacalis] | furnacalis | | | | | | | |
| cadherin-23 | Frankliniella | 105 | 105 | 86% | 7.00E−23 | 26.88 | 1963 | XP_026290496.1 |
| [Frankliniella | occidentalis | | | | | | | |
| occidentalis] | | | | | | | | |
| hypothetical protein | Frankliniella | 105 | 105 | 86% | 7.00E−23 | 26.88 | 1919 | KAE8747974.1 |
| FOCC_FOCC005364 | occidentalis | | | | | | | |
| [Frankliniella | | | | | | | | |
| occidentalis] | | | | | | | | |
| hypothetical protein | Spodoptera | 96.3 | 96.3 | 72% | 6.00E−20 | 26.36 | 1889 | KAG8118266.1 |
| SFRUCORN_019316 | frugiperda | | | | | | | |
| [Spodoptera frugiperda] | | | | | | | | |
| cadherin-23-like | Helicoverpa | 97.4 | 97.4 | 76% | 2.00E−20 | 26.22 | 1803 | XP_021192993.1 |
| [Helicoverpa armigera] | armigera | | | | | | | |
| hypothetical protein | Heliothis | 95.1 | 95.1 | 76% | 1.00E−19 | 26.22 | 787 | PCG68468.1 |
| B5V51_5203 [Heliothis | virescens | | | | | | | |
| virescens] | | | | | | | | |
| hypothetical protein | Heliothis | 94.7 | 94.7 | 76% | 2.00E−19 | 26.22 | 966 | PCG68467.1 |
| B5V51_5203 [Heliothis | virescens | | | | | | | |
| virescens] | | | | | | | | |
| cadherin-23 | Leptinotarsa | 110 | 110 | 91% | 9.00E−25 | 26.07 | 1806 | XP_023023050.1 |
| [Leptinotarsa | decemlineata | | | | | | | |
| decemlineata] | | | | | | | | |
| hypothetical protein | Spodoptera | 74.7 | 74.7 | 72% | 5.00E−13 | 23.51 | 1928 | KAF9810081.1 |
| SFRURICE_011249 | frugiperda | | | | | | | |
| [Spodoptera frugiperda] | | | | | | | | |

Sodium-dependent nutrient amino acid transporter (NAAT)

| | | | | | | | | |
|---|---|---|---|---|---|---|---|---|
| hypothetical protein | Spodoptera | 85.9 | 85.9 | 61% | 1.00E−20 | 100 | 609 | KAG8119417.1 |
| SFRUCORN_006020 | frugiperda | | | | | | | |
| [Spodoptera frugiperda] | | | | | | | | |
| hypothetical protein | Spodoptera | 128 | 128 | 100% | 9.00E−36 | 98.25 | 601 | KAF9824628.1 |
| SFRURICE_004085 | frugiperda | | | | | | | |
| [Spodoptera frugiperda] | | | | | | | | |
| sodium-dependent | Spodoptera | 126 | 126 | 100% | 5.00E−35 | 98.25 | 622 | XP_035432905.1 |
| nutrient amino acid | frugiperda | | | | | | | |
| transporter 1-like | | | | | | | | |
| [Spodoptera frugiperda] | | | | | | | | |
| sodium-dependent | Helicoverpa | 87.8 | 87.8 | 96% | 2.00E−21 | 70.91 | 638 | XP_021190151.1 |
| nutrient amino acid | armigera | | | | | | | |
| transporter 1-like | | | | | | | | |
| [Helicoverpa armigera | | | | | | | | |
| hypothetical protein | Heliothis | 87 | 87 | 96% | 4.00E−21 | 67.27 | 531 | PCG73532.1 |
| B5V51_14736 [Heliothis | virescens | | | | | | | |
| virescens] | | | | | | | | |
| hypothetical protein | Heliothis | 87 | 87 | 96% | 4.00E−21 | 67.27 | 570 | PCG73531.1 |
| B5V51_14736 [Heliothis | virescens | | | | | | | |
| virescens] | | | | | | | | |
| hypothetical protein | Heliothis | 84 | 84 | 96% | 4.00E−20 | 67.27 | 642 | PCG75591.1 |
| B5V51_11338 [Heliothis | virescens | | | | | | | |
| virescens] | | | | | | | | |
| sodium-dependent | Ostrinia | 80.5 | 80.5 | 98% | 8.00E−19 | 62.5 | 567 | XP_028167706.1 |
| nutrient amino acid | furnacalis | | | | | | | |
| transporter 1-like | | | | | | | | |
| [Ostrinia furnacalis] | | | | | | | | |
| sodium-dependent | Ostrinia | 70.1 | 70.1 | 94% | 3.00E−15 | 59.26 | 350 | XP_028167708.1 |
| nutrient amino acid | furnacalis | | | | | | | |
| transporter 1-like | | | | | | | | |
| [Ostrinia furnacalis] | | | | | | | | |
| hypothetical protein | Plutella | 59.7 | 59.7 | 94% | 2.00E−11 | 58.18 | 642 | KAG7302654.1 |
| JYU34_012604 [Plutella | xylostella | | | | | | | |
| xylostella] | | | | | | | | |
| sodium-dependent | Plutella | 57 | 57 | 94% | 2.00E−10 | 56.36 | 636 | XP_037964704.1 |
| nutrient amino acid | xylostella | | | | | | | |
| transporter 1-like | | | | | | | | |
| [Plutella xylostella] | | | | | | | | |
| hypothetical protein | Plutella | 59.3 | 59.3 | 98% | 2.00E−11 | 56.14 | 646 | KAG7306405.1 |
| JYU34_009035 [Plutella | xylostella | | | | | | | |
| xylostella] | | | | | | | | |
| hypothetical protein | Plutella | 67.8 | 67.8 | 96% | 2.00E−14 | 55.36 | 575 | KAG7302653.1 |
| JYU34_012604 [Plutella | xylostella | | | | | | | |
| xylostella] | | | | | | | | |
| sodium-dependent | Plutella | 67.4 | 67.4 | 96% | 4.00E−14 | 55.36 | 638 | XP_037964703.1 |
| nutrient amino acid | xylostella | | | | | | | |
| transporter 1-like | | | | | | | | |
| [Plutella xylostella] | | | | | | | | |
| unnamed protein | Plutella | 66.6 | 66.6 | 96% | 5.00E−14 | 55.36 | 495 | CAG9133367.1 |
| product [Plutella | xylostella | | | | | | | |
| xylostella] | | | | | | | | |
| hypothetical protein | Heliothis | 72.4 | 72.4 | 96% | 5.00E−16 | 54.55 | 574 | PCG73533.1 |
| B5V51_14737 [Heliothis | virescens | | | | | | | |
| virescens] | | | | | | | | |
| sodium-dependent | Helicoverpa | 67 | 67 | 96% | 5.00E−14 | 52.73 | 638 | XP_021190451.1 |
| nutrient amino acid | armigera | | | | | | | |
| transporter 1-like | | | | | | | | |
| Helicoverpa armigera] | | | | | | | | |
| sodium-dependent | Plutella | 57 | 57 | 98% | 1.00E−10 | 51.79 | 626 | XP_037974913.1 |
| nutrient amino acid | xylostella | | | | | | | |
| transporter 1-like | | | | | | | | |
| [Plutella xylostella] | | | | | | | | |
| unnamed protein | Plutella | 57 | 57 | 98% | 1.00E−10 | 51.79 | 642 | CAG9113477.1 |
| product [Plutella | xylostella | | | | | | | |
| xylostella] | | | | | | | | |
| sodium-dependent | Ostrinia | 55.1 | 55.1 | 96% | 9.00E−10 | 50.85 | 641 | XP_028167725.1 |
| nutrient amino acid | furnacalis | | | | | | | |
| transporter 1-like | | | | | | | | |
| [Ostrinia furnacalis] | | | | | | | | |
| sodium-dependent | Spodoptera | 61.6 | 61.6 | 94% | 3.00E−12 | 50 | 641 | XP_035433139.1 |
| nutrient amino acid | frugiperda | | | | | | | |
| transporter 1-like | | | | | | | | |
| [Spodoptera frugiperda] | | | | | | | | |
| hypothetical protein | Spodoptera | 61.6 | 61.6 | 94% | 4.00E−12 | 50 | 664 | KAF9824627.1 |
| SFRURICE_004084 | frugiperda | | | | | | | |
| (Spodoptera frugiperda] | | | | | | | | |
| hypothetical protein | Spodoptera | 61.6 | 61.6 | 94% | 4.00E−12 | 50 | 662 | KAG8119416.1 |
| SFRUCORN_006019 | frugiperda | | | | | | | |
| [Spodoptera frugiperda] | | | | | | | | |
| sodium-dependent | Diabrotica | 47 | 47 | 94% | 6.00E−07 | 46.55 | 638 | XP_028129462.1 |
| nutrient amino acid | virgifera | | | | | | | |
| transporter 1-like | virgifera | | | | | | | |
| [Diabrotica virgifera | | | | | | | | |
| virgifera] | | | | | | | | |
| sodium-dependent | Diabrotica | 51.6 | 51.6 | 94% | 1.00E−08 | 44.83 | 661 | XP_028129463.1 |
| nutrient amino acid | virgifera | | | | | | | |
| transporter 1-like | virgifera | | | | | | | |
| isoform X1 [Diabrotica | | | | | | | | |
| virgifera virgiferal | | | | | | | | |
| sodium-dependent | Diabrotica | 51.2 | 51.2 | 94% | 2.00E−08 | 44.83 | 628 | XP_028129464.1 |
| nutrient amino acid | virgifera | | | | | | | |
| transporter 1-like | virgifera | | | | | | | |
| isoform X2 [Diabrotica | | | | | | | | |
| virgifera virgifera] | | | | | | | | |
| hypothetical protein | Frankliniella | 33.1 | 33.1 | 94% | 0.042 | 35 | 710 | KAE8745179.1 |
| FOCC_FOCC008070 | occidentalis | | | | | | | |
| [Frankliniella | | | | | | | | |
| occidentalis] | | | | | | | | |
| sodium-and chloride- | Frankliniella | 33.1 | 33.1 | 94% | 0.043 | 35 | 642 | XP_026284329.1 |
| dependent GABA | occidentalis | | | | | | | |
| transporter 1-like | | | | | | | | |
| [Frankliniella | | | | | | | | |
| occidentalis] | | | | | | | | |
| nutrient amino acid | Leptinotarsa | 35.8 | 35.8 | 94% | 0.005 | 33.87 | 640 | AHH29249.1 |
| transporter [Leptinotarsa | decemlineata | | | | | | | |
| decemlineata] | | | | | | | | |

Interestingly, insecticidal composition of BsNb specific to Cry1F, when paired with Cry1Ac and Cry1Ab was also effective. Similarly, it is noted that conservation exists in the amino acid sequences between cry toxins (FIGS. 17A and 171B, Table 10).

TABLE 10
Sequence conservation between Cry toxins

| Entry | Protein names | Info | Entry name |
|---|---|---|---|
| P84613 | Insecticidal crystal toxin protein | E-value: 33E−9; | CR4AA_BACTK |
| | | Score: 143; | |
| | | Ident.: 34.5% | |
| P09662 | Pesticidal crystal protein Cry10Aa (78 kDa | E-value: 11E−9; | C10AA_BACTI |
| | crystal protein) (Crystaline entomocidal | Score: 148; | |
| | protoxin) (Insecticidal delta-endotoxin | Ident.: 33.8% | |
| | CryXA(a)) | | |
| Q45754 | Pesticidal crystal protein Cry12Aa (142 kDa | E-value: 7.3; | C12AA_BACTU |
| | crystal protein) (Crystaline entomocidal | Score: 82; | |
| | protoxin) (Insecticidal delta-endotoxin | Ident.: 31.0% | |
| | CryXIIA(a)) | | |
| Q45710 | Pesticidal crystal protein Cry14Aa (132 kDa | E-value: 1.2; | C14AA_BACTS |
| | crystal protein) (Crystaline entomocidal | Score: 88; | |
| | protoxin) (Insecticidal delta-endotoxin | Ident.: 25.0% | |
| | CryXIVA(a)) | | |
| O32307 | Pesticidal crystal protein Cry19Aa (75 kDa | E-value: 0.000002; | C19AA_BACTJ |
| | crystal protein) (Crystaline entomocidal | Score: 131; | |
| | protoxin) (Insecticidal delta-endotoxin | Ident.: 28.3% | |
| | CryXIXA(a)) | | |
| O86170 | Pesticidal crystal protein Cry19Ba (78 kDa | E-value: 40E−12; | C19BA_BACUH |
| | crystal protein) (Crystaline entomocidal | Score: 166; | |
| | protoxin) (Insecticidal delta-endotoxin | Ident.: 36.0% | |
| | CryXIXB(a)) | | |
| P0A366 | Pesticidal crystal protein Cry1Aa (133 kDa | E-value: 40E−54; | CR1AA_BACTK |
| | crystal protein) (Crystaline entomocidal | Score: 478; | |
| | protoxin) (Insecticidal delta-endotoxin | Ident.: 67.9% | |
| | CryIA(a)) | | |
| P0A370 | Pesticidal crystal protein Cry1Ab (130 kDa | E-value: 40E−54; | CR1AB_BACTK |
| | crystal protein) (Crystaline entomocidal | Score: 478; | |
| | protoxin) (Insecticidal delta-endotoxin | Ident.: 67.9% | |
| | CryIA(b)) | | |
| P05068 | Pesticidal crystal protein Cry1Ac (133 kDa | E-value: 9.9E−15; | CR1AC_BACTK |
| | crystal protein) (Crystaline entomocidal | Score: 193; | |
| | protoxin) (Insecticidal delta-endotoxin | Ident.: 39.0% | |
| | CryIA(c)) | | |
| Q03744 | Pesticidal crystal protein Cry1Ad (133 kDa | E-value: 900E−54; | CR1AD_BACTA |
| | crystal protein) (Crystaline entomocidal | Score: 468; | |
| | protoxin) (Insecticidal delta-endotoxin | Ident.: 67.2% | |
| | CryIA(d)) | | |
| Q03748 | Pesticidal crystal protein Cry1Ae (134 kDa | E-value: 6.3E−54; | CR1AE_BACTL |
| | crystal protein) (Crystaline entomocidal | Score: 484; | |
| | protoxin) (Insecticidal delta-endotoxin | Ident.: 68.6% | |
| | CryIA(e)) | | |
| P96315 | Pesticidal crystal protein Cry1Af (Crystaline | E-value: 3E−51; | CR1AF_BACTU |
| | entomocidal protoxin) (Insecticidal delta- | Score: 463; | |
| | endotoxin CryIA(f)) (Fragment) | Ident.: 66.4% | |
| Q9S515 | Pesticidal crystal protein Cry1Ag (134 kDa | E-value: 1.4E−45; | CR1AG_BACTU |
| | crystal protein) (Crystaline entomocidal | Score: 422; | |
| | protoxin) (Insecticidal delta-endotoxin | Ident.: 66.1% | |
| | CryIA(g)) | | |
| P0A373 | Pesticidal crystal protein Cry1Ba (140 kDa | E-value: 11E−42; | CR1BA_BACTK |
| | crystal protein) (Crystaline entomocidal | Score: 393; | |
| | protoxin) (Insecticidal delta-endotoxin | Ident.: 58.1% | |
| | CryIB(a)) | | |
| Q45739 | Pesticidal crystal protein Cry1Bb (140 kDa | E-value: 39E−69; | CR1BB_BACTU |
| | crystal protein) (Crystaline entomocidal | Score: 589; | |
| | protoxin) (Insecticidal delta-endotoxin | Ident.: 84.6% | |
| | CryIB(b)) | | |
| Q45774 | Pesticidal crystal protein Cry1Bc (140 kDa | E-value: 40E−69; | CR1BC_BACTM |
| | crystal protein) (Crystaline entomocidal | Score: 589; | |
| | protoxin) (Insecticidal delta-endotoxin | Ident.: 84.6% | |
| | CryIB(c)) | | |
| Q9ZAZ5 | Pesticidal crystal protein Cry1Bd (140 kDa | E-value: 180E−18; | CR1BD_BACTZ |
| | crystal protein) (Crystaline entomocidal | Score: 206; | |
| | protoxin) (Insecticidal delta-endotoxin | Ident.: 38.7% | |
| | CryIB(d)) | | |
| O85805 | Pesticidal crystal protein Cry1Be (139 kDa | E-value: 990E−24; | CR1BE_BACTU |
| | crystal protein) (Crystaline entomocidal | Score: 245; | |
| | protoxin) (Insecticidal delta-endotoxin | Ident.: 38.5% | |
| | CryIB(e)) | | |
| P0A375 | Pesticidal crystal protein Cry1Ca (134 kDa | E-value: 230E−27; | CR1CA_BACTE |
| | crystal protein) (Crystaline entomocidal | Score: 272; | |
| | protoxin) (Insecticidal delta-endotoxin | Ident.: 41.7% | |
| | CryIC(a)) | | |
| P56953 | Pesticidal crystal protein Cry1Cb (133 kDa | E-value: 6.9E−24; | CR1CB_BACTG |
| | crystal protein) (Crystaline entomocidal | Score: 261; | |
| | protoxin) (Insecticidal delta-endotoxin | Ident.: 40.7% | |
| | CryIC(b)) | | |
| P19415 | Pesticidal crystal protein Cry1Da (132 kDa | E-value: 230E−27; | CR1DA_BACTA |
| | crystal protein) (Crystaline entomocidal | Score: 272; | |
| | protoxin) (Insecticidal delta-endotoxin | Ident.: 45.9% | |
| | CryID(a)) | | |
| Q45747 | Pesticidal crystal protein Cry1Db (131 kDa | E-value: 310E−27; | CR1DB_BACTU |
| | crystal protein) (Crystaline entomocidal | Score: 271; | |
| | protoxin) (Insecticidal delta-endotoxin | Ident.: 45.9% | |
| | CryID(b)) | | |
| Q57458 | Pesticidal crystal protein Cry1Ea (133 kDa | E-value: 90E−27; | CR1EA_BACTX |
| | crystal protein) (Crystaline entomocidal | Score: 275; | |
| | protoxin) (Insecticidal delta-endotoxin | Ident.: 42.3% | |
| | CryIE(a)) | | |
| Q03745 | Pesticidal crystal protein Cry1Eb (134 kDa | E-value: 32E−24; | CR1EB_BACTA |
| | crystal protein) (Crystaline entomocidal | Score: 256; | |
| | protoxin) (Insecticidal delta-endotoxin | Ident.: 40.0% | |
| | CryIE(b)) | | |
| Q03746 | Pesticidal crystal protein Cry1Fa (134 kDa | E-value: 14E−39; | CR1FA_BACTA |
| | crystal protein) (Crystaline entomocidal | Score: 370; | |
| | protoxin) (Insecticidal delta-endotoxin | Ident.: 55.9% | |
| | CryIF(a)) | | |
| O66377 | Pesticidal crystal protein Cry1Fb (132 kDa | E-value: 10E−84; | CR1FB_BACTM |
| | crystal protein) (Crystaline entomocidal | Score: 701; | |
| | protoxin) (Insecticidal delta-endotoxin | Ident.: 100.0% | |
| | CryIF(b)) | | |
| Q45746 | Pesticidal crystal protein Cry1Ga (132 kDa | E-value: 3.7E−60; | CR1GA_BACTU |
| | crystal protein) (Crystaline entomocidal | Score: 530; | |
| | protoxin) (Insecticidal delta-endotoxin | Ident.: 73.7% | |
| | CryIG(a)) | | |
| Q9ZAZ6 | Pesticidal crystal protein Cry1Gb (133 kDa | E-value: 62E−60; | CR1GB_BACTZ |
| | crystal protein) (Crystaline entomocidal | Score: 521; | |
| | protoxin) (Insecticidal delta-endotoxin | Ident.: 74.5% | |
| | CryIG(b)) | | |
| Q45748 | Pesticidal crystal protein Cry1Ha (133 kDa | E-value: 720E−24; | CR1HA_BACTU |
| | crystal protein) (Crystaline entomocidal | Score: 246; | |
| | protoxin) (Insecticidal delta-endotoxin | Ident.: 43.8% | |
| | CryIH(a)) | | |
| Q45718 | Pesticidal crystal protein Cry1Hb (131 kDa | E-value: 310E−27; | CR1HB_BACTM |
| | crystal protein) (Crystaline entomocidal | Score: 271; | |
| | protoxin) (Insecticidal delta-endotoxin | Ident.: 47.8% | |
| | CryIH(b)) | | |
| Q45752 | Pesticidal crystal protein Cry1Ia (81 kDa | E-value: 630E−60; | CR1IA_BACTK |
| | crystal protein) (Crystaline entomocidal | Score: 506; | |
| | protoxin) (Insecticidal delta-endotoxin | Ident.: 73.5% | |
| | CryII(a)) | | |
| Q45709 | Pesticidal crystal protein Cry1Ib (81 kDa | E-value: 120E−60; | CR1IB_BACTE |
| | crystal protein) (Crystaline entomocidal | Score: 511; | |
| | protoxin) (Insecticidal delta-endotoxin | Ident.: 74.3% | |
| | CryII(b)) | | |
| O87404 | Pesticidal crystal protein Cry1Ic (81 kDa | E-value: 46E−54; | CR1IC_BACTU |
| | crystal protein) (Crystaline entomocidal | Score: 472; | |
| | protoxin) (Insecticidal delta-endotoxin | Ident.: 69.1% | |
| | CryII(c)) | | |
| Q9XDL1 | Pesticidal crystal protein Cry1Id (81 kDa | E-value: 1.7E−57; | CR1ID_BACTU |
| | crystal protein) (Crystaline entomocidal | Score: 503; | |
| | protoxin) (Insecticidal delta-endotoxin | Ident.: 72.8% | |
| | CryII(d)) | | |
| Q45738 | Pesticidal crystal protein Cry1Ja (133 kDa | E-value: 770E−81; | CR1JA_BACTU |
| | crystal protein) (Crystaline entomocidal | Score: 666; | |
| | protoxin) (Insecticidal delta-endotoxin | Ident.: 94.9% | |
| | CryIJ(a)) | | |
| Q45716 | Pesticidal crystal protein Cry1Jb (134 kDa | E-value: 1.9E−33; | CR1JB_BACTU |
| | crystal protein) (Crystaline entomocidal | Score: 332; | |
| | protoxin) (Insecticidal delta-endotoxin | Ident.: 48.9% | |
| | CryIJ(b)) | | |
| Q45715 | Pesticidal crystal protein Cry1Ka (137 kDa | E-value: 46E−36; | CR1KA_BACTM |
| | crystal protein) (Crystaline entomocidal | Score: 344; | |
| | protoxin) (Insecticidal delta-endotoxin | Ident.: 51.9% | |
| | CryIK(a)) | | |
| O32321 | Pesticidal crystal protein Cry20Aa (86 kDa | E-value: 0.00039; | C20AA_BACUF |
| | crystal protein) (Crystaline entomocidal | Score: 114; | |
| | protoxin) (Insecticidal delta-endotoxin | Ident.: 29.1% | |
| | CryXXA(a)) | | |
| P56956 | Pesticidal crystal protein Cry21Aa (132 kDa | E-value: 2.2; | C21AA_BACTU |
| | crystal protein) (Crystaline entomocidal | Score: 86; | |
| | protoxin) (Insecticidal delta-endotoxin | Ident.: 27.9% | |
| | CryXXIA(a)) | | |
| O87905 | Pesticidal crystal protein Cry24Aa (Crystaline | E-value: 29E−12; | C24AA_BACTJ |
| | entomocidal protoxin) (Crystal protein) | Score: 167; | |
| | (Insecticidal delta-endotoxin CryXXIVA(a)) | Ident.: 33.8% | |
| | (Insecticidal protein Jeg72) (Fragment) | | |
| O87906 | Pesticidal crystal protein Cry25Aa (76 kDa | E-value: 0.052; | C25AA_BACTJ |
| | crystal protein) (Crystaline entomocidal | Score: 98 | |
| | protoxin) (Insecticidal delta-endotoxin | Ident.: 28.8% | |
| | CryXXVA(a)) (Insecticidal protein Jeg74) | | |
| Q9X597 | Pesticidal crystal protein Cry26Aa (131 kDa | E-value: 830E−18; | C26AA_BACTF |
| | crystal protein) (Crystaline entomocidal | Score: 201; | |
| | protoxin) (Insecticidal delta-endotoxin | Ident.: 36.3% | |
| | CryXXVIA(a)) | | |
| Q9S597 | Pesticidal crystal protein Cry27Aa (94 kDa | E-value: 0.00098; | C27AA_BACUH |
| | crystal protein) (Crystaline entomocidal | Score: 111; | |
| | protoxin) (Insecticidal delta-endotoxin | Ident.: 28.5% | |
| | CryXXVIIA(a)) | | |
| Q9X682 | Pesticidal crystal protein Cry28Aa (126 kDa | E-value: 0.055; | C28AA_BACTF |
| | crystal protein) (Crystaline entomocidal | Score: 98 | |
| | protoxin) (Insecticidal delta-endotoxin | Ident.: 26.7% | |
| | CryXXVIIIA(a)) | | |
| P0A379 | Pesticidal crystal protein Cry3Aa (73 kDa | E-value: 2.6E−27; | CR3AA_BACTT |
| | crystal protein) (Crystaline entomocidal | Score: 286; | |
| | protoxin) (Insecticidal delta-endotoxin | Ident.: 44.9% | |
| | CryIIIA(a)) | | |
| P17969 | Pesticidal crystal protein Cry3Ba (75 kDa | E-value: 4.7E−24; | CR3BA_BACTO |
| | crystal protein) (Crystaline entomocidal | Score: 262; | |
| | protoxin) (Insecticidal delta-endotoxin | Ident.: 42.0% | |
| | CryIIIB(a)) | | |
| Q45744 | Pesticidal crystal protein Cry3Ca (73 kDa | E-value: 2.9E−18; | CR3CA_BACTK |
| | crystal protein) (Crystaline entomocidal | Score: 219; | |
| | protoxin) (Insecticidal delta-endotoxin | Ident.: 38.7% | |
| | CryIIIC(a)) | | |
| P16480 | Pesticidal crystal protein Cry4Aa (135 kDa | E-value: 52E−9; | CR4AA_BACTI |
| | crystal protein) (Crystaline entomocidal | Score: 143; | |
| | protoxin) (Insecticidal delta-endotoxin | Ident.: 34.5% | |
| | CryIVA(a)) | | |
| P05519 | Pesticidal crystal protein Cry4Ba (128 kDa | E-value: 2.4E−9; | CR4BA_BACTI |
| | crystal protein) (Crystaline entomocidal | Score: 153; | |
| | protoxin) (Insecticidal delta-endotoxin | Ident.: 29.4% | |
| | CryIVB(a)) | | |
| Q45760 | Pesticidal crystal protein Cry5Aa (152 kDa | E-value: 1.6; | CR5AA_BACUD |
| | crystal protein) (Crystaline entomocidal | Score: 87; | |
| | protoxin) (Insecticidal delta-endotoxin | Ident.: 28.4% | |
| | CryVA(a)) | | |
| Q45753 | Pesticidal crystal protein Cry5Ab (142 kDa | E-value: 0.14; | CR5AB_BACUD |
| | crystal protein) (Crystaline entomocidal | Score: 95; | |
| | protoxin) (Insecticidal delta-endotoxin | Ident.: 29.6% | |
| | CryVA(b)) | | |
| P56955 | Pesticidal crystal protein Cry5Ac (135 kDa | E-value: 1.2; | CR5AC_BACTU |
| | crystal protein) (Crystaline entomocidal | Score: 88; | |
| | protoxin) (Insecticidal delta-endotoxin | Ident.: 29.7% | |
| | CryVA(c)) | | |
| Q03749 | Pesticidal crystal protein Cry7Aa (129 kDa | E-value: 26E−27; | CR7AA_BACTU |
| | crystal protein) (Crystaline entomocidal | Score: 279; | |
| | protoxin) (Insecticidal delta-endotoxin | Ident.: 44.6% | |
| | CryVIIA(a)) | | |
| Q45708 | Pesticidal crystal protein Cry7Ab (130 kDa | E-value: 310E−27; | CR7AB_BACUK |
| | crystal protein) (Crystaline entomocidal | Score: 271; | |
| | protoxin) (Insecticidal delta-endotoxin | Ident.: 43.9% | |
| | CryVIIA(b)) | | |
| Q45707 | Pesticidal crystal protein Cry7Ab (130 kDa | E-value: 420E−27; | CR7AB_BACUA |
| | crystal protein) (Crystaline entomocidal | Score: 270; | |
| | protoxin) (Insecticidal delta-endotoxin | Ident.: 43.9% | |
| | CryVIIA(b)) | | |
| Q45704 | Pesticidal crystal protein Cry8Aa (131 kDa | E-value: 740E−36; | CR8AA_BACUK |
| | crystal protein) (Crystaline entomocidal | Score: 335; | |
| | protoxin) (Insecticidal delta-endotoxin | Ident.: 52.2% | |
| | CryVIIIA(a)) | | |
| Q45705 | Pesticidal crystal protein Cry8Ba (134 kDa | E-value: 330E−9; | CR8BA_BACUK |
| | crystal protein) (Crystaline entomocidal | Score: 137; | |
| | protoxin) (Insecticidal delta-endotoxin | Ident.: 29.2% | |
| | CryVIIIB(a)) | | |
| Q45706 | Pesticidal crystal protein Cry8Ca (130 kDa | E-value: 0.0064; | CR8CA_BACTP |
| | crystal protein) (Crystaline entomocidal | Score: 105; | |
| | protoxin) (Insecticidal delta-endotoxin | Ident.: 31.3% | |
| | CryVIIIC(a)) | | |
| Q99031 | Pesticidal crystal protein Cry9Aa (130 kDa | E-value: 360E−21; | CR9AA_BACTG |
| | crystal protein) (Crystaline entomocidal | Score: 226; | |
| | protoxin) (Insecticidal delta-endotoxin | Ident.: 39.0% | |
| | CryIXA(a)) | | |
| Q45733 | Pesticidal crystal protein Cry9Ca (130 kDa | E-value: 130E−30; | CR9CA_BACTO |
| | crystal protein) (Crystaline entomocidal | Score: 296; | |
| | protoxin) (Insecticidal delta-endotoxin | Ident.: 45.8% | |
| | CryIXC(a)) | | |
| O06014 | Pesticidal crystal protein Cry9Da (132 kDa | E-value: 700E−45; | CR9DA_BACTP |
| | crystal protein) (Crystaline entomocidal | Score: 402; | |
| | protoxin) (Insecticidal delta-endotoxin | Ident.: 56.6% | |
| | CryIXD(a)) | | |
| Q9ZNL9 | Pesticidal crystal protein Cry9Ea (130 kDa | E-value: 26E−27; | CR9EA_BACTA |
| | crystal protein) (Crystaline entomocidal | Score: 279; | |
| | protoxin) (Insecticidal delta-endotoxin | Ident.: 43.4% | |
| | CryIXE(a)) | | |
| Q45882 | Pesticidal crystal-like protein Cry16Aa | E-value: 0.00015; | C16AA_PARBF |
| | (Cbm71 mosquitocidal toxin) (Insecticidal | Score: 117; | |
| | toxin CryXVIA(a)) | Ident.: 31.8% | |
| O05102 | Pesticidal crystal-like protein Cry17Aa | E-value: 0.038; | C17AA_PARBF |
| | (Cbm72 mosquitocidal toxin) (Insecticidal | Score: 99; | |
| | toxin CryXVIIA(a)) | Ident.: 26.4% | |

The data provided thus far indicates that addition of BsNb's can expand the activity spectrum of insecticidal toxins to insects previously not susceptible by targeting receptors on the insect midgut and by delivering closely related cry toxins.

### Example 26: Activity of Cry Proteins with BsNb

To further investigate the effectiveness of various combinations of Cry proteins and nanobodies activity assays were done. Insect assays were performed on artificial diet in the course of seven days. Samples containing Cry proteins and nanobodies indicated in Table 11 were overlaid on artificial diet and allowed to air dry. Individual neonate insects (one insect per well) were placed in 16 individual wells, sealed and allowed to incubate for seven days at 25° C. After incubation, assessment of mortality on a per well basis was made and percent mortality as compared to appropriate controls was calculated.

Table 11 provides the data for these survival tests.

TABLE 11
Assay design and results

| BsNb # | | | | | Sample | Mor- |
|---|---|---|---|---|---|---|
| (see | | Con- | | | and | tality |
| Table 4) | Protein | centration | Insect | Control | BsNb | increase |
| 4 | Cry1Ab | 0.02 ug/well | VBC | 0% | 31.25% | 31.25% |
| 10 | Cry1Ab | 0.02 ug/well | SCB | 0% | 25.00% | 25.00% |
| 18 | Cry1Ab | 0.02 ug/well | SCB | 0% | 18.75% | 18.75% |
| 108 | Cry1Ab | 0.02 ug/well | VBC | 0% | 18.75% | 18.75% |
| 4 | Cry1Ab | 2.0 ug/well | CEW | 6.25% | 56.25% | 50.00% |
| 4 | Cry1Ab | 2.0 ug/well | FAW | 0% | 25.00% | 25.00% |
| 15 | Cry1Ab | 2.0 ug/well | FAW | 0% | 43.75% | 43.75% |
| 18 | Cry1Ab | 2.0 ug/well | CEW | 6.25% | 56.25% | 50.00% |
| 18 | Cry1Ac | 0.02 ug/well | ECB | 6.25% | 18.75% | 12.50% |
| 15 | Cry1Ac | 0.2 ug/well | CEW | 6.25% | 56.25% | 50.00% |
| 21 | Cry1Ac | 0.2 ug/well | CEW | 6.25% | 43.75% | 37.50% |
| 39 | Cry1Ac | 0.2 ug/well | CEW | 6.25% | 31.25% | 25.00% |
| 62 | Cry1Ac | 0.2 ug/well | CEW | 6.25% | 31.25% | 25.00% |
| 64 | Cry1Ac | 0.2 ug/well | CEW | 6.25% | 37.50% | 31.25% |
| 100 | Cry1Ac | 0.2 ug/well | CEW | 6.25% | 37.50% | 31.25% |
| 111 | Cry1Ac | 0.2 ug/well | CEW | 6.25% | 43.75% | 37.50% |
| 4 | Cry1Ac | 3.37 ug/well | BCW | 12.50% | 31.25% | 18.75% |
| 10 | Cry1Ac | 3.37 ug/well | BCW | 12.50% | 37.25% | 24.75% |
| 62 | Cry1Ac | 3.37 ug/well | BCW | 12.50% | 31.25% | 18.75% |
| 76 | Cry1Ac | 3.37 ug/well | FAW | 6.25% | 25% | 18.75% |
| 100 | Cry1Ac | 3.37 ug/well | BCW | 12.50% | 25% | 12.50% |
| 108 | Cry1F | 1.0 ug/well | TBW | 0.00% | 12.50% | 12.50% |

These data further confirm that the approach provided in the current disclosure not only works to enhance the effectiveness of Cry toxins against their natural targets and resistant variants of their natural targets but also allow for their use to target previously non-susceptible insects, Of particular note is the potential use of Cry1Ab to target CEW when used in conjunction with bispecific nanobody 4, 18 and similarly, Cry1 Ac with 15, 21, 62, 64, 100 and 111.

Combining BsNb's with current transgenic insect control technology and future transgenic insect control technology through transgenic transformation or over the top spray, can enhance activity to insects not traditionally controlled. Table 12 provides an overview of the enhanced activity and expanded spectrum of the nanobodies tested thus far.

TABLE 12
Enhanced activity and spectrum

| | | | Cry1F | | Cry1Ab | | Cry1Ac | |
|---|---|---|---|---|---|---|---|---|
| BsNb | MsNb Cry- | | Enhanced | Enhanced | Enhanced | Enhanced | Enhanced | Enhanced |
| # | link-target | Enhancement | Activity | Spectrum | Activity | Spectrum | Activity | Spectrum |
| 1 | cry1Nb5-218- | None | X | X | X | X | X | X |
| | NAAT29 | | | | | | | |
| 4 | cry1Nb5- | Activity and | DBM | X | VBC | CEW, | DBM, | X |
| | ESGSV- | Spectrum | | | | FAW | BCW | |
| | NAAT29 | | | | | | | |
| 10 | cry1Nb51- | Activity | FAW | X | SCB | X | BCW | X |
| | AEAAAK3- | | | | | | FAW | |
| | NAAT29 | | | | | | | |
| 15 | cry1Nb7- | Activity | X | X | X | FAW | CEW, | X |
| | Gly4Ser1x3- | | | | | | FAW | |
| | NAAT29 | | | | | | | |
| 18 | cry1Nb7- | Activity | FAW | X | SCB | X | FAW | ECB |
| | Gly8- | | | | | | | |
| | NAAT29 | | | | | | | |
| 21 | cry1Nb7- | Activity and | DBM | CEW | X | X | DBM, | X |
| | PTPT- | Spectrum | | | | | CEW | |
| | NAAT29 | | | | | | | |
| 39 | cry1Nb7- | Activity and | FAW | X | X | X | CEW, | X |
| | PTPT- | Spectrum | | | | | FAW | |
| | NAAT31 | | | | | | | |
| 62 | cry1Nb7-PT- | Activity | FAW | X | X | X | CEW, | X |
| | NAAT1 | | | | | | FAW | |
| 64 | cry1Nb7-PT- | Activity | FAW | X | X | X | CEW, | X |
| | NAAT2 | | | | | | FAW, | |
| | | | | | | | BCW | |
| 76 | cry1Nb7-PT- | Activity and | FAW | X | X | FAW | FAW | X |
| | NAAT10 | Spectrum | | | | | | |
| 100 | cry1Nb7-PT- | Activity and | DBM, | CEW | X | X | CEW, | X |
| | Cad2 | Spectrum | FAW | | | | FAW, | |
| | | | | | | | BCW, | |
| | | | | | | | DBM | |
| 108 | cry1Nb7-PT- | Spectrum | X | CEW | VBC | X | X | X |
| | Cad43 | | | | | | | |
| 111 | cry1Nb7- | Activity | DBM, | X | X | X | DBM, | X |
| | Gly8-Cad49 | | FAW | | | | FAW | |
| 112 | cry1Nb7-PT- | Activity | X | X | X | X | DBM, | X |
| | Cad47 | | | | | | TBW | |

### Example 27: Testing Potentially Expanded Activities of Bispecific Nanobodies

Killing insects that are resistant to specific Cry toxins is of significant utility for insect control. To test this, multiple bispecific nanobodies in combination with multiple cry toxins are individually exposed to three different resistant species: Cry1F resistant FAW (rFAW), Cry1Ab resistant SOB (rSCB) and Cry1A.105 resistant CEW (rEW). Samples containing Cry proteins and nanobodies as indicated in Table 13 are overlaid on artificial diet and allowed to air dry. Individual neonate insects (one insect per well) are placed in 16 individual wells, sealed and allowed to incubate for seven days at 2500. After incubation, assessment of mortality on a per well basis is made using a mortality assays as described herein in Example 25 and percent mortality is calculated, as compared to mortality using the cry toxins alone.

TABLE 13
Bispecific nanobodies for testing against
previously resistant insect pests

| Insect | Protein | BsNb |
|---|---|---|
| rSCB-Cry1Ab | Cry1Ab | TBD |
| rCEW-Cry1Ac.105 | Cry1Ac | TBD |
| rCEW-Cry1Ac.105 | Cry1F | TBD |
| rSCB-multi toxin | Multiple | TBD |
| rSCB-Cry2Ab2-RR | Cry2Ab | TBD |
| rFAW-Cry1F-RR | Cry1F | TBD |
| rFAW-Vip3A-RR | Vip3A | TBD |
| rFAW-Cry1/Cry2-RR (dual-gene resistant) | Cry1A and Cry2A | TBD |
| rFAW-Bt-Multi toxin | Multiple | TBD |
| rCEW-Cry1Ab-RR | Cry1Ab | TBD |
| rCEW-Cry2Ab2-RR | Cry2Ab | TBD |
| rCEW-Cry1A/Cry2A-RR (dual-gene | Cry1A and Cry2A | TBD |
| resistant) | | |
| rCEW-Bt-Multi toxin | Multiple | TBD |
| rWCR-Cry3Aa | TBD | TBD |
| rWCR-Cry34/35 | TBD | TBD |
| rWCR- Cry3Aa, Cry34/35 | TBD | TBD |

Sequences

| SEQ. | | | | |
|---|---|---|---|---|
| ID | | Sequence | | Misc. |
| NO. | Organism | type | Sequence | Features |
| 1 | | DNA | GACATTCTGTGGTGAAAACATTTTTTATTT | Cadherin* |
| | | | ATTTTTTTCTAGTGGTTTGTGGGTACAGTG | |
| | | | TAAACATTTTGGAATATTGTTAAAGATTTC | |
| | | | GGAATATTGTTAAAGTATTGACAGATAAAG | |
| | | | CTGTAACATCACTAGAGAAGTGAGAACTGC | |
| | | | AAGATCATGAGATGGCGGTCGATGTGCGAA | |
| | | | TACTGACAGCAACATTGCTGGTACTCACCA | |
| | | | CTGCTACAGCACAGCGAGATCGATGTGGCT | |
| | | | ACATGGTAGAAATACCCAGACCAGACAGGC | |
| | | | CTGACTTCCCACCTCAAAATTTTGACGGTT | |
| | | | TAACATGGGCTCAGCAGCCACTATTACCAG | |
| | | | CTGAGGATCGAGAAGAGGTCTGCCTCAATG | |
| | | | ACTATGAACCTGATCCCTGGAGCAACAACC | |
| | | | ATGGTGACCAGAGAATTTACATGGAGGAGG | |
| | | | AGATCGAAGGTCCCGTAGTCATTGCGAAAA | |
| | | | TTAACTACCAAGGAAACACCCCTCCTCAAA | |
| | | | TAAGATTACCTTTTCGTGTTGGTGCAGCCC | |
| | | | ACATGCTTGGAGCAGAAATTCGTGAATATC | |
| | | | CTGACGCAACTGGAGACTGGTATCTTGTAA | |
| | | | TTACTCAAAGGCAGGACTATGAAACTCCTG | |
| | | | ATATGCAGAGATACACGTTCGATGTGAGTG | |
| | | | TGGAAGGCCAGTCGCTGGTTGTAACGGTGA | |
| | | | GGCTGGATATTGTGAACATCGACGACAATG | |
| | | | CGCCCATCATTGAGATGTTAGAGCCTTGCA | |
| | | | ACTTACCGGAACTTGTTGAACCCCATGTTA | |
| | | | CAGAATGTAAATATATCGTGTCCGACGCAG | |
| | | | ACGGTCTGATCAGTACAAGTGTTATGAGTT | |
| | | | ATCATATAGACAGCGAGAGAGGAGACGAAA | |
| | | | AAGTATTCGAACTGATCAGAAAAGATTATC | |
| | | | CGGGCGATTGGACGAAGGTGTATATGGTTC | |
| | | | TTGAATTGAAAAAATCTCTTGATTACGAAG | |
| | | | AGAATCCTCTACACATATTCAGAGTCACGG | |
| | | | CTTCTGATTCCTTACCAAACAATAGGACCG | |
| | | | TGGTCATGATGGTTGAAGTAGAGAACGTGG | |
| | | | AACATAGAAATCCTCGGTGGATGGAGATCT | |
| | | | TTGCTGTGCAACAGTTTGATGAAAAACAGG | |
| | | | CGAAATCGTTCACAGTGCGAGCTATTGATG | |
| | | | GCGACACGGGAATCAATAAACCTATATTCT | |
| | | | ATCGTATAGAAACTGAAGATGAAGACAAAG | |
| | | | AGTTCTTCAGCATTGAGAACATAGGGGAAG | |
| | | | GCAGAGACGGTGCCAGATTCCACGTGGCTC | |
| | | | CTATAGACAGAGACTACCTGAAAAGGGATA | |
| | | | TGTTTCATATAAGAATAATTGCATATAAAC | |
| | | | AAGGTGATAATGACAAAGAAGGTGAATCAT | |
| | | | CGTTCGAGACCTCAGCAAATGTGACGATTA | |
| | | | TAATTAACGATATAAATGATCAGAGGCCAG | |
| | | | AACCCTTCCATAAAGAATACACGATCTCCA | |
| | | | TAATGGAAGAAACTGCGATGACCTTAGATT | |
| | | | TGCAAGAGTTTGGTTTCCATGACCGTGACA | |
| | | | TTGGTCCCCACGCTCAGTACGACGTTCACT | |
| | | | TAGAGAGTATACAGCCAGAGGGGGCCCATA | |
| | | | CCGCTTTCTACATCGCCCCTGAAGAAGGTT | |
| | | | ACCAGGCCCAGTCTTTCACCATAGGTACTA | |
| | | | GAATCCATAACATGTTGGATTATGAAGATG | |
| | | | ACGACTACAGACCAGGAATAAAGCTAAAGG | |
| | | | CAGTAGCAATTGACAGACACGATAACAATC | |
| | | | ACATTGGGGAAGCAATTATTAACATTAACC | |
| | | | TTATCAATTGGAATGATGAGCTACCTATAT | |
| | | | TCGACGAGGACGCCTACAACGTGACATTTG | |
| | | | AGGAGACGGTCGGTGATGGCTTCCACATTG | |
| | | | GTAAATACCGGGCTAAAGACAGAGACATCG | |
| | | | GTGACATAGTCGAGCACTCGATATTGGGCA | |
| | | | ACGCTGCAAACTTCCTGAGAATTGACATAG | |
| | | | ATACTGGAGATGTGTACGTGTCACGGGACG | |
| | | | ATTACTTTGATTATCAAAGACAGAACGAAA | |
| | | | TCATAGTTCAGATTCTGGCTGTTGATACAC | |
| | | | TAGGTTTACCTCAGAACAGGGCTACCACAC | |
| | | | AGCTCACGATATTTTTGGAAGACATCAACA | |
| | | | ACACGCCACCTATACTGCGACTGCCACGTT | |
| | | | CCAGTCCAAGTGTAGAAGAGAACGTTGAAG | |
| | | | TCGGGCACCCGATTACCGAGGGGCTAACGG | |
| | | | CGACAGACCCAGACACCACAGCCGATTTAC | |
| | | | ACTTCGAGATCGATTGGGACAATTCTTACG | |
| | | | CTACGAAGCAGGGCACCAATGGACCCAACA | |
| | | | CTGCAGACTACCACGGATGCGTAGAAATCC | |
| | | | TGACGGTATACCCAGATCCTGACAATCACG | |
| | | | GGAGAGCTGAGGGTCACTTGGTGGCACGTG | |
| | | | AGGTCAGTGATGGCGTGACCATCGATTACG | |
| | | | AGAAGTTTGAGGTGCTGTACCTCGTCGTCA | |
| | | | GGGTGATAGATCGCAACACTGTCATTGGCC | |
| | | | CTGATTATGACGAAGCAATGCTGACGGTGA | |
| | | | CGATAATCGATATGAACGACAACTGGCCGA | |
| | | | TATGGGCCGACAACACGCTGCAGCAGACAC | |
| | | | TGCGCGTGCGCGAGATGGCCGACGAAGGAG | |
| | | | TCATCGTCGGTACACTGCTCGCCACCGACT | |
| | | | TGGATGGCCCTCTCTACAACCGAGTCCGCT | |
| | | | ACACCATGGTCCCCATCAAGGACACTCCTG | |
| | | | ATGACCTAATAGCGATCAACTACGTCACCG | |
| | | | GTCAGCTGACTGTGAACAAGGGGCAAGCAA | |
| | | | TTGACGCAGATGATCCACCTCGCTTCTACC | |
| | | | TGTATTACAAGGTCACTGCCAGCGATAAGT | |
| | | | GCTCTCTTGACGAGTTCTTCCCTGTGTGCC | |
| | | | CACCTGACCCCACTTACTGGAATACCGAGG | |
| | | | GAGAGATAGCGATCGCGATAACCGATACGA | |
| | | | ACAACAAAATTCCACGCGCGGAAACAGATA | |
| | | | TGTTCCCTAGTGAAAAGCGCATCTATGAGA | |
| | | | ACACACCCAATGGTACCAAGATCACGACGA | |
| | | | TCATCGCTAGTGACCAGGACAGAGATCGAC | |
| | | | CAAATAACGCGCTGACGTACAGAATCAACT | |
| | | | ACGCATTCAACCACAGGCTGGAGAACTTCT | |
| | | | TCGCAGTGGACCCTGATACTGGTGAACTGT | |
| | | | TTGTCCACTTCACCACTAGCGAAGTGTTGG | |
| | | | ACAGAGACGGAGAGGAACCGGAGCATAGGA | |
| | | | TCATCTTCACCATCGTCGATAACTTGGAAG | |
| | | | GCGCTGGAGATGGCAATCAGAACACAATCT | |
| | | | CCACGGAGGTGCGTGTTATACTGCTTGATA | |
| | | | TAAACGACAATAAGCCGGAACTACCAATTC | |
| | | | CTGATGGCGAATTTTGGACCGTTTCCGAAG | |
| | | | GTGAAGTGGAGGGAAAACGCATTCCACCAG | |
| | | | AGATTCACGCACACGACAGAGATGAACCAT | |
| | | | TCAACGACAACTCTCGCGTGGGATATGAAA | |
| | | | TTCGATCGATCAAATTGATCAATAGAGACA | |
| | | | TCGAGCTTCCTCAAGATCCATTCAAAATAA | |
| | | | TAACGATTGATGATCTCGATACCTGGAAAT | |
| | | | TCGTTGGAGAGTTGGAGACTACCATGGACC | |
| | | | TTAGAGGATACTGGGGAACCTATGATGTCG | |
| | | | AGATACGTGCGTTTGACCACGGTTTCCCGA | |
| | | | TGCTGGATTCATTCGAGACCTACCAACTAA | |
| | | | CCGTCAGGCCATACAACTTCCATTCACCGG | |
| | | | TGTTTGTGTTCCCAACTCCTGGCTCAACCA | |
| | | | TCAGGCTTTCTAGGGAGCGTGCTATAGTCA | |
| | | | ATGGTATGCTGGCTCTGGCTAATATCGCGA | |
| | | | GCGGAGAGTTCCTCGACAGACTCTCTGCCA | |
| | | | CTGATGAAGATGGGCTACACGCAGGCAGAG | |
| | | | TAACTTTCTCCATAGCTGGAAACGATGAAG | |
| | | | CTGCGGAATATTTCAATGTGTTGAACGACG | |
| | | | GTGACAACTCAGCAATGCTCACGCTGAAGC | |
| | | | AAGCATTGCCCGCTGGCGTCCAGCAGTTTG | |
| | | | AGTTGGTTATTCGGGCCACGGACGGCGGGA | |
| | | | CGGAGCCGGGACCTAGGAGTACCGACTGCT | |
| | | | CCGTCACTGTGGTGTTTGTGATGACGCAGG | |
| | | | GAGACCCCGTGTTCGACGACAACGCAGCTT | |
| | | | CTGTCCGCTTCGTTGAAAAGGAAGCTGGTA | |
| | | | TGTCGGAAAAGTTTCAGCTGCCTCAGGCCG | |
| | | | ATGACCCCAAAAACTACAGGTGTATGGACG | |
| | | | ACTGCCATACCATCTACTACTCTATCGTTG | |
| | | | ATGGCAACGATGGTGACCACTTCGCCGTGG | |
| | | | AGCCGGAGACTAACGTGATCTATTTGCTGA | |
| | | | AGCCGCTGGACCGCAGCCAACAGGAGCAGT | |
| | | | ACAGGGTCGTGGTGGCGGCTTCCAACACGC | |
| | | | CTGGCGGCACCTCCACCTTGTCCTCCTCAC | |
| | | | TCCTCACCGTCACCATCGGCGTTCGAGAAG | |
| | | | CAAACCCTAGACCGATCTTCGAAAGTGAAT | |
| | | | TTTACACAGCTGGCGTCTTACACACCGATA | |
| | | | GCATACACAAGGAGCTCGTTTACCTGGCGG | |
| | | | CAAAACATTCAGAAGGGCTTCCTATCGTCT | |
| | | | ACTCGATAGATCAAGAAACCATGAAAATAG | |
| | | | ACGAGTCGTTGCAAACAGTTGTGGAGGACG | |
| | | | CCTTCGACATTAACTCTGCAACCGGAGTCA | |
| | | | TATCGCTGAACTTCCAGCCAACATCTGTCA | |
| | | | TGCACGGCAGTTTCGACTTCGAGGTGGTGG | |
| | | | CTAGTGACACGCGTGGAGCGAGTGATCGAG | |
| | | | CAAAAGTGTCAATTTACATGATATCGACTC | |
| | | | GCGTTAGAGTAGCCTTCCTGTTCTACAACA | |
| | | | CGGAAGCTGAAGTTAACGAGAGAAGAAATT | |
| | | | TCATTGCACAAACGTTCGCCAACGCGTTTG | |
| | | | GTATGACATGTAACATAGACAGCGTGCTGC | |
| | | | CGGCTACCGACGCCAACGGCGTGATTCGCG | |
| | | | AGGGGTACACAGAACTCCAGGCTCACTTCA | |
| | | | TACGAGACGACCAGCCGGTGCCAGCCGACT | |
| | | | ATATTGAGGGATTATTTACGGAACTCAATA | |
| | | | CATTGCGTGACATCAGAGAGGTACTGAGTA | |
| | | | CTCAGCAATTGACGCTACTGGACTTTGCGG | |
| | | | CGGGAGGGTCGGCAGTGCTGCCCGGCGGAG | |
| | | | AGTACGCGCTAGCGGTGTACATCCTCGCCG | |
| | | | GCATCGCAGCGTTACTCGCCGTCATCTGTC | |
| | | | TCGCTCTCCTCATCGCTTTCTTCATTAGGA | |
| | | | ACCGAACACTGAACCGGCGCATCGAAGCCC | |
| | | | TCACAATCAAAGATGTTCCTACGGACATCG | |
| | | | AGCCAAACCACGCGTCAGTAGCAGTGCTAA | |
| | | | ACATTAACAAGCACACAGAACCTGGTTCCA | |
| | | | ATCCCTTCTATAACCCGGATGTTAAGACAC | |
| | | | CTAACTTCGACACTATAAGCGAAGTATCCG | |
| | | | ATGACCTGCTTGATGTCGAAGACTTGGAAC | |
| | | | AGTTTGGAAAGGATTACTTCCCACCCGAAA | |
| | | | ACGAAATTGAGAGCCTGAATTTTGCACGTA | |
| | | | ACCCCATAGCGACACACGGGAACAACTTTG | |
| | | | GCGTAAACTCAAGCCCCTCCAACCCAGAGT | |
| | | | TCTCCAACTCCCAGTTTAGAAGTTAAACTA | |
| | | | AATACACTTTTATCACTTGCATAGACTTAT | |
| | | | GTATTTAATAATTTTACATTTTTTACATTA | |
| | | | AATATAAATGTTTTATATGTAATAATAGTG | |
| | | | TGATAAAATGTACGTAACAATCAACATAGC | |
| | | | TGTTGTAGGTTCGTAAATAACATACTCGTA | |
| | | | ATGTATAAGTGTTATGTTTATATATAGAAA | |
| | | | TAAAAATATTAAATATTAAAAAAAAAAAAA | |
| | | | AAAAAAAAAAAA | |
| 2 | Spodoptera | Protein | MAVDVRILTATLLVLTTATAQRDRCGYMVE | Cadherin |
| | frugiperda | | IPRPDRPDFPPQNFDGLTWAQQPLLPAEDR | |
| | | | EEVCLNDYEPDPWSNNHGDQRIYMEEEIEG | |
| | | | PVVIAKINYQGNTLIGVSVVLPPQIRLPFR | |
| | | | VGAAHMLGAEIREYPDATGDWYLVITQRQD | |
| | | | YETPDMQRYTFDVSVEGQSLVVTVRLDIVN | |
| | | | IDDNAPIIEMLEPCNLPELVEPHVTECKYI | |
| | | | VSDADGLISTSVMSYHIDSERGDEKVFELI | |
| | | | RKDYPGDWTKVYMVLELKKSLDYEENPLHI | |
| | | | FRVTASDSLPNNRTVVMMVEVENVEHRNPR | |
| | | | WMEIFAVQQFDEKQAKSFTVRAIDGDTGIN | |
| | | | KPIFYRIETEDEDKEFFSIENIGEGRDGAR | |
| | | | FHVAPIDRDYLKRDMFHIRIIAYKQGDNDK | |
| | | | EGESSFETSANVTIIINDINDQRPEPFHKE | |
| | | | YTISIMEETAMTLDLQEFGFHDRDIGPHAQ | |
| | | | YDVHLESIQPEGAHTAFYIAPEEGYQAQSF | |
| | | | TIGTRIHNMLDYEDDDYRPGIKLKAVAIDR | |
| | | | HDNNHIGEAIININLINWNDELPIFDEDAY | |
| | | | NVTFEETVGDGFHIGKYRAKDRDIGDIVEH | |
| | | | SILGNAANFLRIDIDTGDVYVSRDDYFDYQ | |
| | | | RQNEIIVQILAVDTLGLPQNRATTQLTIFL | |
| | | | EDINNTPPILRLPRSSPSVEENVEVGHPIT | |
| | | | EGLTATDPDTTADLHFEIDWDNSYATKQGT | |
| | | | NGPNTADYHGCVEILTVYPDPDNHGRAEGH | |
| | | | LVAREVSDGVTIDYEKFEVLYLVVRVIDRN | |
| | | | TVIGPDYDEAMLTVTIIDMNDNWPIWADNT | |
| | | | LQQTLRVREMADEGVIVGTLLATDLDGPLY | |
| | | | NRVRYTMVPIKDTPDDLIAINYVTGQLTVN | |
| | | | KGQAIDADDPPRFYLYYKVTASDKCSLDEF | |
| | | | FPVCPPDPTYWNTEGEIAIAITDTNNKIPR | |
| | | | AETDMFPSEKRIYENTPNGTKITTIIASDQ | |
| | | | DRDRPNNALTYRINYAFNHRLENFFAVDPD | |
| | | | TGELFVHFTTSEVLDRDGEEPEHRIIFTIV | |
| | | | DNLEGAGDGNQNTISTEVRVILLDINDNKP | |
| | | | ELPIPDGEFWTVSEGEVEGKRIPPEIHAHD | |
| | | | RDEPFNDNSRVGYEIRSIKLINRDIELPQD | |
| | | | PFKIITIDDLDTWKFVGELETTMDLRGYWG | |
| | | | TYDVEIRAFDHGFPMLDSFETYQLTVRPYN | |
| | | | FHSPVFVFPTPGSTIRLSRERAIVNGMLAL | |
| | | | ANIASGEFLDRLSATDEDGLHAGRVTFSIA | |
| | | | GNDEAAEYFNVLNDGDNSAMLTLKQALPAG | |
| | | | VQQFELVIRATDGGTEPGPRSTDCSVTVVF | |
| | | | VMTQGDPVFDDNAASVRFVEKEAGMSEKFQ | |
| | | | LPQADDPKNYRCMDDCHTIYYSIVDGNDGD | |
| | | | HFAVEPETNVIYLLKPLDRSQQEQYRVVVA | |
| | | | ASNTPGGTSTLSSSLLTVTIGVREANPRPI | |
| | | | FESEFYTAGVLHTDSIHKELVYLAAKHSEG | |
| | | | LPIVYSIDQETMKIDESLQTVVEDAFDINS | |
| | | | ATGVISLNFQPTSVMHGSFDFEVVASDTRG | |
| | | | ASDRAKVSIYMISTRVRVAFLFYNTEAEVN | |
| | | | ERRNFIAQTFANAFGMTCNIDSVLPATDAN | |
| | | | GVIREGYTELQAHFIRDDQPVPADYIEGLF | |
| | | | TELNTLRDIREVLSTQQLTLLDFAAGGSAV | |
| | | | LPGGEYALAVYILAGIAALLAVICLALLIA | |
| | | | FFIRNRTLNRRIEALTIKDVPTDIEPNHAS | |
| | | | VAVLNINKHTEPGSNPFYNPDVKTPNFDTI | |
| | | | SEVSDDLLDVEDLEQFGKDYFPPENEIESL | |
| | | | NFARNPIATHGNNFGVNSSPSNPEFSNSQF | |
| | | | RS | |
| 3 | | DNA | ATGGCAGTCGACGTGAGAATATTCACGGCA | Cadherin |
| | | | GCGGTTTTTATACTCGCTGCTCACTTCACT | |
| | | | TTCGCACAAGATTGTAGCTACATGGTAGCA | |
| | | | ATACCCAGACCAGAGCGACCAGATTTTCCA | |
| | | | AGTCAAAATTTCGATGGAATACCATGGAGT | |
| | | | CAGTATCCCTTGATACCAGTGGAGGGTAGA | |
| | | | GAAGACGTGTGTATGAACGAGTTCGAGCCA | |
| | | | GGTAACCAAAACCCTGTTACCGTCATCTTC | |
| | | | ATGGAAGAGGAGATCGAAGGGGATGTGGCC | |
| | | | ATCGCACGGCTCAACTATCGAGGTACCAAT | |
| | | | ACTCCGACCATTGTATCTCCATTTAGCTTT | |
| | | | GGTACTTTTAACATGTTGGGGCCGGTCATA | |
| | | | CGTAGAATACCTGAGAATGGTGGTGACTGG | |
| | | | CATCTCGTCATTACACAGAGACAGGACTAC | |
| | | | GAGACACCAGGTATGCAGCAGTACATCTTC | |
| | | | GACGTGAGGGTAGACGACGAACCCCTGGTG | |
| | | | GCCACGGTCATGCTTCTCATCGTCAACATT | |
| | | | GATGACAACGATCCTATCATACAGATGTTC | |
| | | | GAGCCTTGTGATATTCCGGAACGCGGTGAA | |
| | | | ACAGGCATCACATCATGCAAGTATACAGTC | |
| | | | AGCGATGCTGACGGTGAGATCAGTACTCGC | |
| | | | TTCATGAGGTTCGAAATTTCAAGCGATCGA | |
| | | | GACGATGACGAATATTTCGAACTCGTCAGG | |
| | | | GAAAATATACAAGGACAGTGGATGTATGTT | |
| | | | CATATGAGAGTTCACGTCAAAAAACCTCTT | |
| | | | GACTACGAGGAAAACCCGCTACATTTGTTT | |
| | | | AGAGTTACAGCTTATGATTCCCTACCAAAC | |
| | | | ACACATACAGTAACAATGATGGTGCAAGTA | |
| | | | GAGAACGTTGAGAACAGACCGCCGCGATGG | |
| | | | GTGGAGATATTTGCTGTCCAGCAGTTCGAT | |
| | | | GAGAAGACGGAGCGGTCCTTCAGGGTTCGA | |
| | | | GCCATCGATGGTGATACAGGAATCGATAAA | |
| | | | CCTATCTTCTATAGGATCGAAACAGAAGAA | |
| | | | GGAGAGGAAAACTTGTTCAGCATTCAAACA | |
| | | | ATGGAAGGTGGTCGAGAAGGAGCTTGGTTT | |
| | | | AACGTTGCTCCAATAGACAGAGACACTCTT | |
| | | | GAGAAGGAAGTTTTCCACGTGTCCATAATA | |
| | | | GCGTACAAATATGGTGATAATGACGTGGAA | |
| | | | GGCAGTTCGTCGTTCCAGTCGAAAACCGAT | |
| | | | GTGGTCATCATCGTGAACGATGTCAATGAT | |
| | | | CAGGCTCCGTTGCCTTTCCGGGAAGAGTAT | |
| | | | TCCATTGAAATTATGGAGGAAACTGCGATG | |
| | | | ACACTGAACTTAGAAGACTTTGGGTTCCAC | |
| | | | GATAGAGATCTTGGTCCTCACGCCCAATAC | |
| | | | ACAGTGCACCTAGAGAGCATCCATCCCCCC | |
| | | | CGAGCTCACGAGGCGTTCTACATAGCACCG | |
| | | | GAGGTGGGCTACCAGCGCCAGTCCTTCATC | |
| | | | ATGGGCACGCAGAACCATCACATGCTGGAC | |
| | | | TTTGAAGTGCCGGAGTTCCAGAATATACAA | |
| | | | CTGAGGGCTATAGCGATAGACATGGACGAT | |
| | | | CCCAAATGGGTTGGTATTGCGATAATCAAC | |
| | | | ATTAAACTGATCAACTGGAACGATGAGCTG | |
| | | | CCGATGTTCGAGAGTGACGTGCAAACCGTC | |
| | | | AGCTTCGACGAGACAGAGGGCGCTGGCTTC | |
| | | | TATGTGGCCACTGTTGTGGCGAAGGACCGG | |
| | | | GATGTTGGTGATAAAGTCGAACACTCTCTA | |
| | | | ATGGGTAACGCAGTAAGCTACCTGAGGATC | |
| | | | GACAAGGAAACCGGCGAGATATTCGTCACA | |
| | | | GAAAACGAAGCTTTCAACTATCACAGACAG | |
| | | | AACGAACTCTTTGTGCAGATACGAGCTGAT | |
| | | | GACACATTAGGCGAGCCATACAACACCAAC | |
| | | | ACTACCCAGTTGGTGATCAAGCTGCGGGAT | |
| | | | ATTAACAACACTCCTCCTACGCTCAGACTG | |
| | | | CCTCGCGCCACTCCGTCAGTGGAAGAGAAC | |
| | | | GTGCCCGACGGGTTTGTGATCCCCACACAA | |
| | | | CTGAACGCCACGGACCCCGACACTACAGCC | |
| | | | GAGCTGCGCTTCGAGATCGACTGGGAGAAC | |
| | | | TCCTATGCCACCAAGCAGGGACGGAATACT | |
| | | | GACTCTAAGGAGTATATAGGTTGTATAGAA | |
| | | | ATCGAGACGATATACCCGAATATAAACCAG | |
| | | | CGCGGCAACGCCATCGGCCGCGTGGTGGTG | |
| | | | CGAGAGATCCGGGACGGCGTCACCATAGAC | |
| | | | TATGAGATGTTTGAAGTGCTATATCTGACG | |
| | | | GTCATTGTGAGGGATCTCAACACTGTTATT | |
| | | | GGGGAAGACCATGATATTTCCACATTCACA | |
| | | | ATCACAATAATAGACATGAACGACAACCCT | |
| | | | CCCCTGTGGGTGGAAGGCACCCTCACTCAA | |
| | | | GAGTTCCGCGTGCGAGAGGTCGCAGCCTCA | |
| | | | GGAGTCGTTATAGGATCCGTACTGGCTACT | |
| | | | GATATCGACGGACCGCTGTATAATCAAGTG | |
| | | | CAGTATACTATAACTCCCAGACTCGATACT | |
| | | | CCAGAAGACCTAGTGGACATAGACTTCAAC | |
| | | | ACGGGTCAGATCTCCGTGAAGTTACACCAA | |
| | | | GCTATAGATGCAGACGAGCCGCCGCGTCAG | |
| | | | AACCTCTACTACACCGTCATAGCTAGTGAC | |
| | | | AAGTGTGACCTCCTCACTGTCACTGGGTGT | |
| | | | CCTCCTGACCCTACCTACTTTGGGACGCCG | |
| | | | GGAGAGATCACGATCCACATAACGGACACG | |
| | | | AACAACAAGGTGCCTCAAGTGGAAGACGAC | |
| | | | AAGTTCGAGGCGACGGTGTACATCTACGAG | |
| | | | GGCGCGGACGACGGAGAACACGTCGTGCAG | |
| | | | ATCTACGCCAGCGATCTCGATAGAGATGAA | |
| | | | ATCTACCACAAAGTGAGCTACCAGATCAAC | |
| | | | TACGCGATCAACTCCCGTCTCCGCGACTTC | |
| | | | TTCGAGATGGACCTGGAGACAGGCCTGGTG | |
| | | | TACGTCAACAACACCGCCGGCGAGCTGCTG | |
| | | | GACAGGGACGGCGACGAGCCCACACATCGC | |
| | | | ATCTTCTTCAATGTCATCGATAACTTCTAT | |
| | | | GGAGAAGGAGATGGCAACCGCAATCAGAAC | |
| | | | GAGACACAAGTATTGGTAGTATTGCTGGAC | |
| | | | ATCAACGACAACTATCCAGAACTGCCTGAA | |
| | | | ACAATCCCATGGGCTATCTCTGAGAGCTTA | |
| | | | GAGCAGGGTGAGCGAGTACCGCCAGAAATC | |
| | | | TTCGCCCGGGACCGCGATGAACCCGGAACA | |
| | | | GACAACTCCCGCGTCGCCTACGCCATCACC | |
| | | | GGTCTTACCAGCACCGACCGGGACATACAA | |
| | | | GTGCCTGATCTCTTCAACATGATCACCATA | |
| | | | GAGAGGGACAGGGGAATTGATCAAACTGGA | |
| | | | ATACTTGAGGCAGCTATGGATTTGCGGGGC | |
| | | | TATTGGGGCACTTATGAAATTGATATACAG | |
| | | | GCGTACGACCATGGCATACCTCGAAGGATT | |
| | | | TCAAATCAGAAGTACCCGCTGGTCATCAGA | |
| | | | CCTTACAACTTCCACGACCCAGTGTTCGTG | |
| | | | TTCCCTCAACCTGGATCTACTATCAGACTG | |
| | | | GCAAAGGAGCGAGCAGTAGTCAACGGTATA | |
| | | | CTGGCCACAGTGGACGGCGAATTTCTGGAC | |
| | | | AGAATCGTCGCCACCGACGAAGATGGTTTA | |
| | | | GAAGCTGGACTTGTTACATTCTCTATCGCG | |
| | | | GGAGATGATGAAGATGCTCAGTTCTTCGAC | |
| | | | GTGTTGAACGATGGAGTGAACTCGGGCGCT | |
| | | | CTCACCCTCACGCGGCTCTTCCCTGAAGAT | |
| | | | TTCCGAGAGTTCCAGGTGACGATTCGTGCT | |
| | | | ACGGACGGTGGAACTGAGCCTGGTCCAAGG | |
| | | | AGTACGGACTGTGCGGTGACCGTAGTGTTT | |
| | | | GTTCCCACACAGGGAGAGCCCGTGTTCGAG | |
| | | | GAAAGTACCTACACGGTCGCTTTTGTTGAA | |
| | | | AAAGATGAGGGTATGGAGGAGAGGGCAGAA | |
| | | | TTACCTCGCGCCTCAGATCCGAGGAACATC | |
| | | | ATGTGTGAAGATGACTGTCACGACACCTAT | |
| | | | TACAGCATTGTTGGAGGCAATTCGGGTGAA | |
| | | | CACTTCAGAGTAGACCCTCGTACCAACGTG | |
| | | | CTGACCCTCGTGAAGCCGCTGGACCGCTCC | |
| | | | GAACAGGAGACACACACCCTCATCATCGGA | |
| | | | GCCAGCGACACTCCCAACCCGGCCGCCGTC | |
| | | | CTGCAGGCTTCTACACTCACTGTCACTGTT | |
| | | | AATGTTCGAGAAGCGAACCCGCGACCAGTG | |
| | | | TTCCAGAGAGCACTCTACACAGCTGGCATC | |
| | | | TCTGCTGGCGATTTCATCGAAAGAAATCTG | |
| | | | CTGACTGTAGTAGCGACACATTCAGAAGGT | |
| | | | CTGCCCATCACTTACACTCTGATACAAGAG | |
| | | | TCCATGGAAGCAGACCCCACACTCGAAGCT | |
| | | | GTTCAGGAGTCAGCCTTCATCCTCAACCCT | |
| | | | GAGACTGGAGTCCTGTCACTCAACTTCCAG | |
| | | | CCAACCGCCGCCATGCATGGCATGTTTGAG | |
| | | | TTCGAAGTCGAAGCCACTGATTCAAGGAGA | |
| | | | GAAACTGCCCGCACGGAAGTGAAGGTGTAC | |
| | | | GTGATATCGGACCGCAACCGAGTGTTCTTC | |
| | | | ACGTTCAATAACCCGCTGCCTGAAGTCACA | |
| | | | CCCCAGGAAGATTTCATAGCGGAGACGTTC | |
| | | | ACGGCATTCTTCGGCATGACGTGCAACATC | |
| | | | GACCAGACGTGGTGGGCCAGCGACCCCGTC | |
| | | | ACCGGCGCCACCAGGGACGACCAGACTGAA | |
| | | | GTCAGGGCTCATTTCATCAGGGACGACTTA | |
| | | | CCCGTGCCTGCTGAGGAGATTGAACAGTTA | |
| | | | CGCGGTAACCCAACTCTAGTAAATAGCATC | |
| | | | CAACGAGCCCTAGAAGAACAGAACCTGCAG | |
| | | | CTGGCCGACCTGTTCACGGGCGAGACGCCC | |
| | | | ATCCTGGGGGGTGACGCGCAGGCGCGAGCT | |
| | | | CTGTACGCGCTGGCGGCGGTGGCTGCTGCA | |
| | | | CTCGCGCTGATTGTAGTTGTGCTGCTTATT | |
| | | | GTGTTCTTTGTTAGGACTAGGACTCTGAAC | |
| | | | CGGCGCTTGCAAGCTCTATCCATGACCAAG | |
| | | | TACAGTTCGCAAGACTCGGGGCTGAACCGC | |
| | | | GTGGGTCTGGCGGCGCCGGGCACCAACAAG | |
| | | | CACGCCGTCGAGGGCTCCAACCCCATCTGG | |
| | | | AACGAAACGTTGAAGGCTCCAGACTTTGAT | |
| | | | GCTCTTAGTGAGCAGTCATACGACTCAGAC | |
| | | | CTGATCGGCATCGAAGACTTGCCGCAGTTC | |
| | | | AGGAACGACTACTTCCCGCCTGAAGAGGGC | |
| | | | AGCTCCATGCGAGGAGTCGTCAATGAACAC | |
| | | | GTGCCTGAATCAATAGCGAACCATAACAAC | |
| | | | AACTTCGGGTTCAACTCTACTCCCTTCAGC | |
| | | | CCAGAGTTCGCGAACACACAGTTCGGAAGA | |
| | | | TAA | |
| 4 | Helicoverpa | Protein | MAVDVRIFTAAVFILAAHFTFAQDCSYMVA | Cadherin |
| | armigera | | IPRPERPDFPSQNFDGIPWSQYPLIPVEGR | |
| | | | EDVCMNEFEPGNQNPVTVIFMEEEIEGDVA | |
| | | | IARLNYRGTNTPTIVSPFSFGTFNMLGPVI | |
| | | | RRIPENGGDWHLVITQRQDYETPGMQQYIF | |
| | | | DVRVDDEPLVATVMLLIVNIDDNDPIIQMF | |
| | | | EPCDIPERGETGITSCKYTVSDADGEISTR | |
| | | | FMRFEISSDRDDDEYFELVRENIQGQWMYV | |
| | | | HMRVHVKKPLDYEENPLHLFRVTAYDSLPN | |
| | | | THTVTMMVQVENVENRPPRWVEIFAVQQFD | |
| | | | EKTERSFRVRAIDGDTGIDKPIFYRIETEE | |
| | | | GEENLFSIQTMEGGREGAWFNVAPIDRDTL | |
| | | | EKEVFHVSIIAYKYGDNDVEGSSSFQSKTD | |
| | | | WVIIVNDVNDQAPLPFREEYSIEIMEETAM | |
| | | | TLNLEDFGFHDRDLGPHAQYTVHLESIHPP | |
| | | | RAHEAFYIAPEVGYQRQSFIMGTQNHHMLD | |
| | | | FEVPEFQNIQLRAIAIDMDDPKWVGIAIIN | |
| | | | IKLINWNDELPMFESDVQTVSFDETEGAGF | |
| | | | YVATVVAKDRDVGDKVEHSLMGNAVSYLRI | |
| | | | DKETGEIFVTENEAFNYHRQNELFVQIRAD | |
| | | | DTLGEPYNTNTTQLVIKLRDINNTPPTLRL | |
| | | | PRATPSVEENVPDGFVIPTQLNATDPDTTA | |
| | | | ELRFEIDWENSYATKQGRNTDSKEYIGCIE | |
| | | | IETIYPNINQRGNAIGRVVVREIRDGVTID | |
| | | | YEMFEVLYLTVIVRDLNTVIGEDHDISTFT | |
| | | | ITIIDMNDNPPLWVEGTLTQEFRVREVAAS | |
| | | | GVVIGSVLATDIDGPLYNQVQYTITPRLDT | |
| | | | PEDLVDIDFNTGQISVKLHQAIDADEPPRQ | |
| | | | NLYYTVIASDKCDLLTVTGCPPDPTYFGTP | |
| | | | GEITIHITDTNNKVPQVEDDKFEATVYIYE | |
| | | | GADDGEHVVQIYASDLDRDEIYHKVSYQIN | |
| | | | YAINSRLRDFFEMDLETGLVYVNNTAGELL | |
| | | | DRDGDEPTHRIFFNVIDNFYGEGDGNRNQN | |
| | | | ETQVLVVLLDINDNYPELPETIPWAISESL | |
| | | | EQGERVPPEIFARDRDEPGTDNSRVAYAIT | |
| | | | GLTSTDRDIQVPDLFNMITIERDRGIDQTG | |
| | | | ILEAAMDLRGYWGTYEIDIQAYDHGIPRRI | |
| | | | SNQKYPLVIRPYNFHDPVFVFPQPGSTIRL | |
| | | | AKERAVVNGILATVDGEFLDRIVATDEDGL | |
| | | | EAGLVTFSIAGDDEDAQFFDVLNDGVNSGA | |
| | | | LTLTRLFPEDFREFQVTIRATDGGTEPGPR | |
| | | | STDCAVTVVFVPTQGEPVFEESTYTVAFVE | |
| | | | KDEGMEERAELPRASDPRNIMCEDDCHDTY | |
| | | | YSIVGGNSGEHFRVDPRTNVLTLVKPLDRS | |
| | | | EQETHTLIIGASDTPNPAAVLQASTLTVTV | |
| | | | NVREANPRPVFQRALYTAGISAGDFIERNL | |
| | | | LTVVATHSEGLPITYTLIQESMEADPTLEA | |
| | | | VQESAFILNPETGVLSLNFQPTAAMHGMFE | |
| | | | FEVEATDSRRETARTEVKVYVISDRNRVFF | |
| | | | TFNNPLPEVTPQEDFIAETFTAFFGMTCNI | |
| | | | DQTWWASDPVTGATRDDQTEVRAHFIRDDL | |
| | | | PVPAEEIEQLRGNPTLVNSIQRALEEQNLQ | |
| | | | LADLFTGETPILGGDAQARALYALAAVAAA | |
| | | | LALIVVVLLIVFFVRTRTLNRRLQALSMTK | |
| | | | YSSQDSGLNRVGLAAPGTNKHAVEGSNPIW | |
| | | | NETLKAPDFDALSEQSYDSDLIGIEDLPQF | |
| | | | RNDYFPPEEGSSMRGVVNEHVPESIANHNN | |
| | | | NFGFNSTPFSPEFANTQFGR | |
| 5 | Diabrotica | DNA | AGAGCATATCGAAAGATAATAAAGTTAATC | Cadherin |
| | virgifera | | ACAGGTATAAAACTTTTTTCATGAAATCTG | |
| | virgifera | | TAAAATATTTAGCACTAAACTATTTGTTTT | |
| | | | TGTGAGAGTTACAAGTGAGAGGGTGTTTTG | |
| | | | TAAATGATGTGGATTGTTTGATACCCTTAA | |
| | | | AAAACATCAAAAATGGCTACGAGAAATCTA | |
| | | | TGTTTATGTATGCTTATTTGGATGCCACTC | |
| | | | TTTGAGGGCATAGTTGGCGACGTCGCCTTT | |
| | | | GGCATTGCACAAATTCCTGGTGGTAAGGCA | |
| | | | ACAGTAGAAGATTTGAAAAAAGGGAAATAC | |
| | | | TTACTTAACATGGAAGAAAATAATCATGGC | |
| | | | GGCGTAATACCAACTCCACTTTTTACTATT | |
| | | | ACAGGGGTGGACAACACTGATTGTCCTAAC | |
| | | | TTGAATGTTGAATTTACTCAAGGCATGAAG | |
| | | | TTTAAGTTTTCGATTAATGAAAGTTGTATA | |
| | | | TTCTATGCTGAACAGACATTTGACTACGAA | |
| | | | TCGAATGAAAGATCATACAATTTCAGAATT | |
| | | | TCAAAGTCCGTAAATGACGAGATAGATATT | |
| | | | GCATTCAGTATCAAAAATATCGATGATGAA | |
| | | | CCTCCACAATTAGGAACGTTTAAATGCAAC | |
| | | | TTTAATGAACAATTAGACTATAGTCTTGAT | |
| | | | GACACACCATGCAATACTACATTGCATGAT | |
| | | | CCCGACGGATGGCTGGCACAAGAGAAAATT | |
| | | | TTAATATTCATCGACACTAAGGATGAAGAT | |
| | | | ATATTCGCAATTGATTTGCGAAAGCCTCTA | |
| | | | CCAAATGATACTGGGACAGACACCTACGTT | |
| | | | TTTATGTATCTCTTAAAGCAACTTAATTAT | |
| | | | GAGGATACCAATTTTTATCAGTTTACAGTG | |
| | | | CAAGCAAATGATTCTGGAGGTAATCTTTCA | |
| | | | CCACAAGAAAGTGCTGTGGTAAATGTTATC | |
| | | | AACATTAGAAGTAGACCTCCAAAATGGTCA | |
| | | | AAAATAACATTATTTGATCAATTTGACGAA | |
| | | | CTGACCGAACAAGATTACGATATACAAGCT | |
| | | | TTAGATGGAGATACTGGAATTCATGCAGAT | |
| | | | ATTTGTTATGCAAAACTAGGTGAAGATTTA | |
| | | | CCAGACAATTACATAAACGTCAGTACCGAT | |
| | | | AAAACCAATAAAAAAGGCCATATTCACGTT | |
| | | | AATCCCATTGATAGAGATAAGGATGATCTA | |
| | | | ACACTATACCATTTTAACATTTCTGCCTAT | |
| | | | GTTTGTGATGCCCCAGACTATTTTACTGTA | |
| | | | AATACAGTGCAGTATTACATCATCGATATT | |
| | | | GACAATAATCCTCCCAGGATAGTTGAAAAT | |
| | | | GTTGGGGATGACGGAAAGAACACAACATTT | |
| | | | GACGATGATAACGACCATAAAAATGTTACA | |
| | | | CTAAGTTATTTGGAAAATTATTCGAGATCG | |
| | | | TATAATTTTAGCACAACCATTACAGATAGA | |
| | | | GATACGGGCGAAAACGCGCAATTCACTGTA | |
| | | | AGTCTCGAAAATGTTGAGGGTTCTACGGTT | |
| | | | GATTATACACAACCCTATCTAATAGTACCG | |
| | | | GACAATGCTTACAAGACAGGATCATTCATA | |
| | | | ATAAGCGTGAAAAATAAGACCTTCCTGGAT | |
| | | | TTCGAGAACGACACTTGGAAAAAGCATTCC | |
| | | | TATTATGTTGTTTCCAATGGAAAAAAAGAC | |
| | | | AAATCAAAGACTGACAGAATGTTAATATCA | |
| | | | GTCTCACTAGAAGACTATAACGATGAACTT | |
| | | | CCTATTTTCGAGAAAGAATCATACACAACA | |
| | | | GAAATAAATGAGACTGTTGCGAACGGCACT | |
| | | | CAAATACTATACACCCATGCAACAGACAGA | |
| | | | GATGCAGAAGATTTCGAACTGAAAATGAAT | |
| | | | ATAGTTGGAACTTATGCTGAGAATAGGCTA | |
| | | | AGTATTGATAAAGATGGAAATATTAAAGTA | |
| | | | GAGGTAGTCAATGCCTTCGATTACGATGTC | |
| | | | TTAAATTCTGTGTATTTCCAAGTAACCGCA | |
| | | | ACAGATAAGGTTAATCATGTGACTAGAGTA | |
| | | | CCAGTAACGATTAACATTTTGGATGTTAAT | |
| | | | AATGAAGCTCCCGTAATAAATCAAGTGGAC | |
| | | | CATATACAAATCGAAGAAAACCAACAAAAC | |
| | | | GGTGTAGTGCTAAATGTAACAATAACAGCT | |
| | | | ACGGATGTAGATACCACGGCTAATCTGACT | |
| | | | GCTACTATAAATTGGGAAGATTCCAAAGTT | |
| | | | ACCAAAACTAATGGTGCTGTAGTAAAAACT | |
| | | | GACGCTGTAATAAAAGCTATGCAATTTTTA | |
| | | | GAGATAGAAAATACACAAACAGATGACGGT | |
| | | | CTGGAAATGAAACTAAAGGTAATAAATAAT | |
| | | | AATGACGATAATCCAGACAAGCCAGATTTT | |
| | | | GAAACTTTCGATACATTGTATTTAAGTATA | |
| | | | GTTGTTGAAGACCGTAATACTGATCCTGAT | |
| | | | TTTGAACAAAATCGATATACAGAAGGTCAA | |
| | | | ATTGTCATCAATATCCTTGATGTTAACGAT | |
| | | | AATCCACCATATTTTCCACCTTCTAATGAT | |
| | | | GACACTCGACAGGTCCAGGAAATGTCTCTG | |
| | | | AAAGGAGTATCGGTTGGCTCCATAAAAGCT | |
| | | | GTCGATGTTGATTTGAATTCAGAAATTACC | |
| | | | TACCATTGCACGCCCGAATATGAAAAGTTT | |
| | | | GACTGGGTCGATGTAAATCTTACAACTGGC | |
| | | | GCCATAACTGTAAAGAACGATAAACAAGTT | |
| | | | GACGCAGACACAGACAAAACGTATTATTTC | |
| | | | AACTATACTTGTTGGGCTCATGATGGTGTG | |
| | | | TTCTTTTCCAAACCGTTAGACATCTCAATC | |
| | | | TATGTCATCGACACAAACAACGAAGTGCCT | |
| | | | GTAATAGATTTTCCAAATGAAGTACACGTA | |
| | | | AAGGAAAAATCACTTATAGATACAGTAATT | |
| | | | AAAAAGATTGTTACAAGTGATTTAGATAGA | |
| | | | GATGAGCCGTTTCGTACTGTAAACTGTAAT | |
| | | | TTTGCAAGCGATACTGATCCAGACTGTCAA | |
| | | | ATAGAATTCTATATTGATACCAATGTTCTT | |
| | | | AAAGTGAAAAGAAATAAAACTCTTGATCGT | |
| | | | GATAAGGGAAGAAAAACATATCCTTGTCTA | |
| | | | TTTGAGTGCTTGGATAATCCTTTAAATGTT | |
| | | | CGATCCCAAGGACAAAATAAAGCCAATAAA | |
| | | | AGCTTTACTATAATATTGGATGATATAAAT | |
| | | | GATCATGCACCTGTGCTTATGACGAAGGAC | |
| | | | CTACAATGTTCTGAAAATTTGAATAAGGAC | |
| | | | GGCGAAGTAGGAGAGGGCATAATAGGAGAA | |
| | | | GATATTGATGATGGTGATAATGCTAAAATA | |
| | | | GATTTTTCTGTGTTAAGTATCGTTGATAAG | |
| | | | GAAACCAAAAATGACATTCAAGAATCTTTT | |
| | | | AATATTTCTAAAATTGATAGTGATTATGTA | |
| | | | TTGAACGATACATTGAAGAAAGTGCATCTT | |
| | | | ATAGCCTTCGAAGATCTCAAAGGAAAATAT | |
| | | | GGAACGTATGAAGTCACTTTACACATGCAT | |
| | | | GACGAAGGAGATCCAATGCAGACAACAGAT | |
| | | | CCGGATCCAACTTTAACACTTACAATTGAA | |
| | | | AAATGGAATTACCAAACACCAAGTATAATA | |
| | | | TTTCCTGAAAACGATCAAACATACATTGTC | |
| | | | CTATCGGATCAACAACCTGGTCAACCATTG | |
| | | | GCACTGTTTAACAACACTGGAACTTCAAAT | |
| | | | ACTTTGCCCGATTTTAGTGCAACAGATGGT | |
| | | | GAAACGAAGGACTATTCTAAGTGGGATGTG | |
| | | | AAATTTTCTTATACGCAGACTAATTATGAA | |
| | | | GATGATAAAATTTTCGTGATAGACCATATA | |
| | | | CAGCCGTGCGTTTCTCAACTACAAGTCAGC | |
| | | | AAACATTTCAACTCGGATCTAGTACGAAGT | |
| | | | AAAAAGTATAAGCTAACTATTACAGCCAGT | |
| | | | GTTAAGGATGGAGCAGAACAGGAAGGTGAA | |
| | | | GCAGGTTACTCGACATCAGCAAATATAAGT | |
| | | | ATAGTTTTCCTCAACAACGACGCTCAGCCG | |
| | | | ATCTTTCAAAATAGTGACTGGAGTGTATCA | |
| | | | TTTGTTGAATTCAATACAACACAACCTGCG | |
| | | | AAACCTTTGGAAGAACAGGCTGAGTATGAG | |
| | | | AACACGAAAGGAGGACTTCCTATCTATTAC | |
| | | | CATTTCTATTCCGAAAACCAGACCCTTTCC | |
| | | | AAATATTTTGAGGTTGATGAGACGTCCGGT | |
| | | | GATTTATCGGTTATAGGAAATCTTACCTAT | |
| | | | GATTATGATCAAGATATATCGTTTCACATA | |
| | | | GTTGCTTCGAATGACTCACAAGTCAGAATG | |
| | | | TTGGATCCACGATCCAGTCTAAATGTTACT | |
| | | | GTTAATTTTCTTCCACGTAACCGCAGAGCT | |
| | | | CCTCAGTGGAAAAGTACCAAATTCTTTGGA | |
| | | | GCAGTTATGCCTACATTCGTAACTGGGAAC | |
| | | | CTCATAGTCACCGCTCAAGCTCATGACGAC | |
| | | | GATTATATCGATCAACAACGCGGATTAACG | |
| | | | TGTTCTATATCTAGTGAGATTAACCGAATT | |
| | | | GGGGAAGGATTAGATAAAATAATAGGCGAA | |
| | | | CCATTTTATTTGAGCACTGAAAATGATGCC | |
| | | | GCCAAAATATTTTTGGACTTTACAGTGCAG | |
| | | | ACTACAATGACCGGTCGATTTGAATTTAAA | |
| | | | ATCAAAGTAGAGGACAATAGAGATGACTAC | |
| | | | GGCAATGGTCCATTTGAAAGTGAAGCTGAT | |
| | | | ACAACAATATTTATTATTACTAAAGACAAC | |
| | | | ACCGTTGATTTTCAATTTTACAACGATATT | |
| | | | GAGGATGTTCAGGACAGAGAAACACCGATG | |
| | | | TTAAAAATAATATCCGATATAGTGGGATAT | |
| | | | GATGCATATCGTCAAAATATAGATACAGTA | |
| | | | ACAAATAGCGGTCTCGTTAGGACCAGAGCA | |
| | | | AGGCTTTATTTCATTGACAGTAAAAGCAGC | |
| | | | CGTCGGTTGCAACTCACTAAAAGTCCCGCA | |
| | | | GACAGTGGATTCGAACTGGTTAATAGTGAA | |
| | | | ACAATATTAAACATAGTGACCAATGTGAAC | |
| | | | ACCTTCCAAAATCTGGCCAGCACCTTGAGA | |
| | | | AGTGAACAAAAACTGAACCTCGACAGCTTT | |
| | | | GAAACGAATTCCAAATCCGGCAACAGCGAA | |
| | | | GCAGCACTAAGAGCTTGGTTGATCGGAGTG | |
| | | | TCCGTGGTCTTGGGGATCTTGGTCTTGCTT | |
| | | | TTACTAATCACTTTAATACTAAAAACCAGA | |
| | | | CAGTTAAGTAACAGAATTAAAAAACTAACT | |
| | | | ACCCCCCAATTTGGTTCTCAAGAGTCTGGA | |
| | | | CTTAACAGGATGGGCATAAATGCACCCACC | |
| | | | ACCAACAAGCATGCCATCGAAGGAACAAAT | |
| | | | CCAGTATACAATAATAACGAAATCAAGAAG | |
| | | | CCAAAAAATATGAACGATTTTGATACTCAC | |
| | | | AGCATAAGAAGCGGTGATTCTGATTTGGTT | |
| | | | GGAATAGAAAACAACCCAGAGTTTGACTAC | |
| | | | AACTTCAACACTAACGAGGATAAAACTACA | |
| | | | TATCTCTAAACTATTTTTATAATAATCTTA | |
| | | | TGTCTAGCTTAAGTTGGTCTTAATAGATAT | |
| | | | TAATGTAATCTGATTTAACTATAATTGTAT | |
| | | | TATAAGTATTAAATATGTATAAAATAAACA | |
| | | | CTTATTAGATCCAAAAAAAAAAAAAAAAAA | |
| | | | A | |
| 6 | Diabrotica | protein | MATRNLCLCMLIWMPLFEGIVGDVAFGIAQ | Cadherin |
| | virgifera | | IPGGKATVEDLKKGKYLLNMEENNHGGVIP | |
| | virgifera | | TPLFTITGVDNTDCPNLNVEFTQGMKFKFS | |
| | | | INESCIFYAEQTFDYESNERSYNFRISKSV | |
| | | | NDEIDIAFSIKNIDDEPPQLGTFKCNFNEQ | |
| | | | LDYSLDDTPCNTTLHDPDGWLAQEKILIFI | |
| | | | DTKDEDIFAIDLRKPLPNDTGTDTYVFMYL | |
| | | | LKQLNYEDTNFYQFTVQANDSGGNLSPQES | |
| | | | AVVNVINIRSRPPKWSKITLFDQFDELTEQ | |
| | | | DYDIQALDGDTGIHADICYAKLGEDLPDNY | |
| | | | INVSTDKTNKKGHIHVNPIDRDKDDLTLYH | |
| | | | FNISAYVCDAPDYFTVNTVQYYIIDIDNNP | |
| | | | PRIVENVGDDGKNTTFDDDNDHKNVTLSYL | |
| | | | ENYSRSYNFSTTITDRDTGENAQFTVSLEN | |
| | | | VEGSTVDYTQPYLIVPDNAYKTGSFIISVK | |
| | | | NKTFLDFENDTWKKHSYYVVSNGKKDKSKT | |
| | | | DRMLISVSLEDYNDELPIFEKESYTTEINE | |
| | | | TVANGTQILYTHATDRDAEDFELKMNIVGT | |
| | | | YAENRLSIDKDGNIKVEVVNAFDYDVLNSV | |
| | | | YFQVTATDKVNHVTRVPVTINILDVNNEAP | |
| | | | VINQVDHIQIEENQQNGVVLNVTITATDVD | |
| | | | TTANLTATINWEDSKVTKTNGAVVKTDAVI | |
| | | | KAMQFLEIENTQTDDGLEMKLKVINNNDDN | |
| | | | PDKPDFETFDTLYLSIVVEDRNTDPDFEQN | |
| | | | RYTEGQIVINILDVNDNPPYFPPSNDDTRQ | |
| | | | VQEMSLKGVSVGSIKAVDVDLNSEITYHCT | |
| | | | PEYEKFDWVDVNLTTGAITVKNDKQVDADT | |
| | | | DKTYYFNYTCWAHDGVFFSKPLDISIYVID | |
| | | | TNNEVPVIDFPNEVHVKEKSLIDTVIKKIV | |
| | | | TSDLDRDEPFRTVNCNFASDTDPDCQIEFY | |
| | | | IDTNVLKVKRNKTLDRDKGRKTYPCLFECL | |
| | | | DNPLNVRSQGQNKANKSFTIILDDINDHAP | |
| | | | VLMTKDLQCSENLNKDGEVGEGIIGEDIDD | |
| | | | GDNAKIDFSVLSIVDKETKNDIQESFNISK | |
| | | | IDSDYVLNDTLKKVHLIAFEDLKGKYGTYE | |
| | | | VTLHMHDEGDPMQTTDPDPTLTLTIEKWNY | |
| | | | QTPSIIFPENDQTYIVLSDQQPGQPLALFN | |
| | | | NTGTSNTLPDFSATDGETKDYSKWDVKFSY | |
| | | | TQTNYEDDKIFVIDHIQPCVSQLQVSKHFN | |
| | | | SDLVRSKKYKLTITASVKDGAEQEGEAGYS | |
| | | | TSANISIVFLNNDAQPIFQNSDWSVSFVEF | |
| | | | NTTQPAKPLEEQAEYENTKGGLPIYYHFYS | |
| | | | ENQTLSKYFEVDETSGDLSVIGNLTYDYDQ | |
| | | | DISFHIVASNDSQVRMLDPRSSLNVTVNFL | |
| | | | PRNRRAPQWKSTKFFGAVMPTFVTGNLIVT | |
| | | | AQAHDDDYIDQQRGLTCSISSEINRIGEGL | |
| | | | DKIIGEPFYLSTENDAAKIFLDFTVQTTMT | |
| | | | GRFEFKIKVEDNRDDYGNGPFESEADTTIF | |
| | | | IITKDNTVDFQFYNDIEDVQDRETPMLKII | |
| | | | SDIVGYDAYRQNIDTVTNSGLVRTRARLYF | |
| | | | IDSKSSRRLQLTKSPADSGFELVNSETILN | |
| | | | IVTNVNTFQNLASTLRSEQKLNLDSFETNS | |
| | | | KSGNSEAALRAWLIGVSVVLGILVLLLLIT | |
| | | | LILKTRQLSNRIKKLTTPQFGSQESGLNRM | |
| | | | GINAPTTNKHAIEGTNPVYNNNEIKKPKNM | |
| | | | NDFDTHSIRSGDSDLVGIENNPEFDYNFNT | |
| | | | NEDKTTYL | |
| 7 | Heliothis | DNA | ATGGCAGTCGACGTGAGAATAGTAACGGCA | Cadherin |
| | virescens | | GCGGTATTGATTCTCGCTGCTAATTTAACT | |
| | | | TTCGCGCAAGATTGTTCCTATATGGTAGCA | |
| | | | ATTCCCAGACCAGAGCGACCTGACTTTCCT | |
| | | | AATCAAAATTTCGAAGGAGTACCATGGAGT | |
| | | | CAGAACCCCCTGTTACCAGCGGAGGATAGG | |
| | | | GAAGATGTGTGCATGAACGCGTTTGATCCA | |
| | | | AGTGCCTTGAACCCCGTCACCGTCATCTTC | |
| | | | ATGGAGGAGGAGATCGAAGGGGACGTGGCC | |
| | | | ATTGCCAGGCTTAACTACCGAGGTACCAAT | |
| | | | ACTCCGACCGTGGTAACTCCATTTAACTTT | |
| | | | GGTACCTTCCACTTGTTGGGGCCGGTCATA | |
| | | | CGTAGGATCCCCGAGCAAGGGGGGGACTGG | |
| | | | CATCTTGTTATTACGCAGAGGCAGGACTAT | |
| | | | GAGACCCCGAACATGCAGCAGTATATCTTC | |
| | | | AACGTGAGAGTAGAGGATGAGCCCCAGGAA | |
| | | | GCCACTGTGATGCTCATCATTGTCAACATC | |
| | | | GACGACAACGCTCCTATCATACAGATGTTC | |
| | | | GAGCCTTGTGACATTCCTGAACACGGCGAA | |
| | | | ACCGGCACCACAGAATGCAAGTACGTAGTG | |
| | | | AGCGATGCTGACGGCGAGATCAGCACACGT | |
| | | | TTCATGACGTTTGAAATCGAGAGCGATCGA | |
| | | | AACGACGAAGAATATTTCGAACTCGTGAGA | |
| | | | GAGAATATTCAGGGACAGTGGATGTACGTC | |
| | | | CATATGAGGCTTATACTCAACAAACCTCTT | |
| | | | GACTATGAGGAAAACCCGCTGCATTTGTTT | |
| | | | AGAGTTACAGCTTTGGATTCCCTACCAAAC | |
| | | | GTTCATACAGTGACGATGATGGTGCAAGTC | |
| | | | GAGAACATAGAGAGCAGACCACCGCGGTGG | |
| | | | ATGGAGACCTTCGCCGTGCAGCAGTTCGAT | |
| | | | GAGAAGACAGCACAAGCCTTCAGGGTTCGA | |
| | | | GCCATCGATGGAGACACGGGAATCGATAAA | |
| | | | CCTATTTTCTATAGGATTGAAACTGAAGAA | |
| | | | AGCGAGAAAGATTTGTTCAGTGTTGAAACA | |
| | | | ATAGGAGCTGGTCGAGAAGGTGCTTGGTTT | |
| | | | AAAGTCGCTCCAATAGACAGAGACACTCTT | |
| | | | GAAAAGGAAGTTTTCCACGTGTCTCTAATA | |
| | | | GCGTACAAATATGGCGACAATGACGTGGAA | |
| | | | GGAAGTTCGTCATTCGAGTCGAAAACCGAT | |
| | | | ATCGTCATTATTGTGAACGACGTGAATGAT | |
| | | | CAGGCGCCGGTGCCTTTCCGTCCTTCATAC | |
| | | | TTCATTGAAATTATGGAGGAAACTGCGATG | |
| | | | ACATTGAATTTAGAGGACTTTGGTTTCCAC | |
| | | | GATAGAGATCTTGGTCCGCACGCGCAGTAC | |
| | | | ACGGTACACCTGGAGAGCATCTCCCCGGCG | |
| | | | GGAGCGCACGAGGCGTTCTACATCGCGCCG | |
| | | | GAGGTGGGCTACCAGCGACAGTCCTTCATC | |
| | | | GTCGGCACGCAGAACCATCACATGCTGGAC | |
| | | | TTCGAAGTGCCAGAGTTCCAGAAGATACAA | |
| | | | CTTAGGGCAGTAGCCATAGACATGGACGAT | |
| | | | CCCAGGTGGGTTGGTATCGCGATTATAAAC | |
| | | | ATTAACCTGATCAACTGGAACGATGAGCTG | |
| | | | CCGATCTTCGAGCACGATGTGCAGACTGTG | |
| | | | ACCTTCAAGGAGACGGAGGGCGCTGGCTTC | |
| | | | CGGGTCGCCACTGTTCTGGCAAAGGACAGG | |
| | | | GATATTGATGATAGAGTCGAACATTCTCTA | |
| | | | ATGGGCAACGCAGTGAATTACCTGAGTATC | |
| | | | GACAAAGACACCGGTGACATCCTCGTGACA | |
| | | | ATTGACGATGCATTCAACTATCACAGACAG | |
| | | | AACGAGCTCTTTGTGCAGATACGAGCTGAC | |
| | | | GACACGTTGGGAGAGCCGTATAATACGAAC | |
| | | | ACTGCCCAACTGGTGATACAGCTGCAAGAC | |
| | | | ATCAATAACACACCTCCAACGCTCAGACTG | |
| | | | CCCCGCACGACTCCGTCAGTGGAAGAGAAC | |
| | | | GTGCCGGACGGGTTCGTGATCCCCACCGAG | |
| | | | CTGCACGCCTCCGACCCCGACACCACCGCC | |
| | | | GAGCTGCGCTTCAGCATCGACTGGGACACT | |
| | | | TCCTATGCCACCAAGCAGGGCAGGGATGCT | |
| | | | GATGCTAAGGAGTTTGTTAATTGCATAGAA | |
| | | | ATCGAGACGGTATACCCGAACTTGAACGAC | |
| | | | CGAGGCACCGCCATCGGCCGCGTGGTGGTT | |
| | | | CGCGAGATCCGGGAACACGTCACTATAGAC | |
| | | | TACGAGATGTTCGAGGTGCTGTACCTCACC | |
| | | | GTCAGGGTCACGGATCTCAACACGGTCATT | |
| | | | GGAGACGACTATGATATATCAACATTCACG | |
| | | | ATCATAATAATAGACATGAACGACAACCCT | |
| | | | CCGCTGTGGGTGGAAGGCACGCTGACGCAG | |
| | | | GAGTTCCGCGTGCGAGAGGTCGCCGCCTCA | |
| | | | GGAGTTGTTATAGGATCCGTACTCGCCACT | |
| | | | GATATTGATGGACCTCTTTATAATCAAGTG | |
| | | | CGGTATACCATCACTCCTAGATTAGACACT | |
| | | | CCAGAAGACCTAGTGGAGATCGACTTCAAT | |
| | | | TCGGGTCAGATCTCAGTGAAGAAGCACCAG | |
| | | | GCTATCGACGCGGACGAGCCGCCGCGCCAG | |
| | | | CACCTCTACTGCACCGTGGTCGCCAGCGAC | |
| | | | AAGTGCGACCTGCTCTCTGTCGACGTCTGT | |
| | | | CCGCCTGACCCTAACTACTTCAACACACCG | |
| | | | GGTGAAATAACGATCCACATAACAGACACG | |
| | | | AACAACAAGGTGCCTCGAGTGGAGGAGGAC | |
| | | | AAGTTCGACGAAACCGTCTATATCTACGAG | |
| | | | GGCGCGGAGGACGGAGAACAAGTCGTGCAG | |
| | | | CTCTTCGCCAGCGATCTGGATAGAGATGAA | |
| | | | ATCTACCACAAAGTGAGCTACCAGACCAAC | |
| | | | TACGCGATCAACCCTCGTCTCCGCGACTTC | |
| | | | TTCGAGGTAGACCTGGAGACCGGTCTGGTG | |
| | | | TACGTCAACAACACGGCCGGGGAGAAGCTC | |
| | | | GACCGGGACGGCGATGAACCCACGCATCGG | |
| | | | ATCTTCTTCAACGTCATCGATAACTTCTAT | |
| | | | GGGGAAGGAGACGGCAACCGGAACCAGGAC | |
| | | | GAGACCCAAGTGTTAGTGGTGCTGTTGGAC | |
| | | | ATCAACGACAACTATCCGGAACTGCCTGAG | |
| | | | GGTCTCTCATGGGATATCTCTGAGAGCTTG | |
| | | | CTACAGGGTGTCCGTGTAACCCCAGATATC | |
| | | | TTCGCCCCGGACCGCGACGAGCCCGGCACC | |
| | | | GACAACTCCCGCGTGGCGTACGACATCGTC | |
| | | | AGCCTCACGCCCACCGACAGGGACATCACA | |
| | | | CTTCCTCAACTCTTCACCATGATCACCATA | |
| | | | GAGAAGGACAGGGGCATCGACCAGACTGGA | |
| | | | GAACTGGAGACCGCTATGGATTTAAGAGGC | |
| | | | TATTGGGGCACTTATGAAATACATGTCAAG | |
| | | | GCATACGACCATGGAGTACCTCAAAGGATT | |
| | | | TCCTACGAGAAGTACCCGCTAGTTATAAGA | |
| | | | CCTTACAACTTCCACGATCCTGTGTTTGTG | |
| | | | TTCCCTCAACCTGGAATGACTATCAGACTC | |
| | | | GCGAAGGAGCGAGCAGTAGTGAACGGCGTG | |
| | | | CTGGCGACAGTGGACGGCGAGTTCCTGGAG | |
| | | | CGAATCGTCGCTACCGACGAGGACGGCTTA | |
| | | | CACGCTGGAGTTGTCACCTTCTCTATCTCG | |
| | | | GGAGATGATGAGGCGTTGCAGTACTTCGAC | |
| | | | GTGTTTAACGACGGAGTGAACTTAGGTGCG | |
| | | | CTGACCATCACGCAGCTCTTCCCTGAAGAC | |
| | | | TTCCGAGAGTTTCAGGTGACGATTCGTGCT | |
| | | | ACGGATGGTGGTACGGAGCCTGGTCCAAGG | |
| | | | AGTACGGACTGCACCGTCACCGTAGTGTTT | |
| | | | GTTCCTACGCAGGGAGAGCCTGTGTTCGAG | |
| | | | ACAAGCACCTACACGGTCGCTTTTATTGAG | |
| | | | AAAGATGCTGGTATGGAGGAACGGGCTACG | |
| | | | CTGCCTCTCGCCAAGGACCCGCGGAACATA | |
| | | | ATGTGTGAAGATGATTGTCACGACACCTAT | |
| | | | TACAGCATTGTTGGAGGCAACTCGATGGGC | |
| | | | CACTTTGCAGTGGACCCCCAGTCCAACGAG | |
| | | | CTGTTCCTGCTGACACCGCTGGACCGCGCG | |
| | | | GAGCAGGAGACGCACACCCTCATCATCGGC | |
| | | | GCCAGCGACTCGCCCAGCCCGGCCGCCGTG | |
| | | | CTGCAGGCTTCCACCCTCACTGTTACTGTC | |
| | | | AATGTTCGAGAAGCAAATCCGCGGCCAGTG | |
| | | | TTCCAGAGCGCTCTGTACACAGCCGGCATC | |
| | | | TCCACCCTCGACACCATCAACAGAGCTCTG | |
| | | | CTGACACTACACGCGACACATTCAGAAGGC | |
| | | | CTGCCCGTGACCTACACGCTGATACAAGAC | |
| | | | TCCATGGAAGCTGACTCCACACTGCAAGCT | |
| | | | GTGCAGGAGACAGCCTTCAACCTCAACCCT | |
| | | | CAGACTGGAGTGCTGACCCTCAACTTCCAG | |
| | | | CCAACTGCCTCCATGCACGGCATGTTTGAG | |
| | | | TTCGATGTGATGGCTATTGATACAGTGGGA | |
| | | | GAAACCGCACGCACCGAAGTGAAGGTGTAC | |
| | | | CTGATATCCGACCGCAACAGAGTGTTCTTC | |
| | | | ACGTTCATGAACACGCTCGAAGAAGTCGAA | |
| | | | CCGAATGAGGATTTCATGGCGGAGACATTT | |
| | | | ACCCTGTTCTTCGGCATGCGGTGCAACATC | |
| | | | GACCAGACGCTGCCCGCCAGTGACCCCGCC | |
| | | | ACCGGCGCCGCCAGGGACGACCAGACCGAA | |
| | | | GTCAGGGCACACTTCATACGCGACGACCTG | |
| | | | CCTGTGCCGGCTGAGGAGATCGAACAGTTG | |
| | | | CGCGGTAATCCAACCCTAGTGGCGACAATC | |
| | | | CAGAACGCCCTGCAGGAGGAGAACCTGAAC | |
| | | | CTGGCCGACCTGTTCACGGGCGAGACTCCC | |
| | | | ATCCTGGGCGGCGAGGCGCAGGCGCGGGCG | |
| | | | GTGTACGCGCTGGCGGCGGTGGCGGCTGCG | |
| | | | CTCGCGCTGCTCTGTGTCGTACTGCTTATA | |
| | | | CTCTTCTTCATCAGGACTAGGGCCCTCAAC | |
| | | | CGTCGCCTGGAAGCTCTCTCCATGACCAAG | |
| | | | TATAGTTCCCAAGACTCAGGACTAAACCGC | |
| | | | GTGGGTCTGGCGGCGCCGGGCACCAACAAG | |
| | | | CACGCGGTGGAGGGCTCCAACCCCATCTGG | |
| | | | AACGAAACCCTCAAGGCACCGGACTTTGAT | |
| | | | GCTCTTAGCGAGCAGTCGTACGACTCGGAC | |
| | | | CTAATCGGCATTGAAGACTTGCCGCAGTTC | |
| | | | AGGAACGACTACTTCCCGCCTGACGAGGAG | |
| | | | AGCTCCATGCGGGGAGTCGTCAATGAACAC | |
| | | | ATGCCTGGAGCTAATTCAGTAGCAAACCAT | |
| | | | AACAATAACTTCGGGTTCAACGCTACCCCC | |
| | | | TTTAGCCCAGAGTTCGCGAACTCGCAGCTC | |
| | | | AGAAGATAAAATATTATAGTATTTTTTATA | |
| | | | CAATATTATATAGAAGTGATATAACGCACT | |
| | | | AAAATTTACCTATAAGTATGGGCGAAG | |
| 8 | Heliothis | Protein | MAVDVRIVTAAVLILAANLTFAQDCSYMVA | Cadherin |
| | virescens | | IPRPERPDFPNQNFEGVPWSQNPLLPAEDR | |
| | | | EDVCMNAFDPSALNPVTVIFMEEEIEGDVA | |
| | | | IARLNYRGTNTPTVVTPFNFGTFHLLGPVI | |
| | | | RRIPEQGGDWHLVITQRQDYETPNMQQYIF | |
| | | | NVRVEDEPQEATVMLIIVNIDDNAPIIQMF | |
| | | | EPCDIPEHGETGTTECKYVVSDADGEISTR | |
| | | | FMTFEIESDRNDEEYFELVRENIQGQWMYV | |
| | | | HMRLILNKPLDYEENPLHLFRVTALDSLPN | |
| | | | VHTVTMMVQVENIESRPPRWMETFAVQQFD | |
| | | | EKTAQAFRVRAIDGDTGIDKPIFYRIETEE | |
| | | | SEKDLFSVETIGAGREGAWFKVAPIDRDTL | |
| | | | EKEVFHVSLIAYKYGDNDVEGSSSFESKTD | |
| | | | IVIIVNDVNDQAPVPFRPSYFIEIMEETAM | |
| | | | TLNLEDFGFHDRDLGPHAQYTVHLESISPA | |
| | | | GAHEAFYIAPEVGYQRQSFIVGTQNHHMLD | |
| | | | FEVPEFQKIQLRAVAIDMDDPRWVGIAIIN | |
| | | | INLINWNDELPIFEHDVQTVTFKETEGAGF | |
| | | | RVATVLAKDRDIDDRVEHSLMGNAVNYLSI | |
| | | | DKDTGDILVTIDDAFNYHRQNELFVQIRAD | |
| | | | DTLGEPYNTNTAQLVIQLQDINNTPPTLRL | |
| | | | PRTTPSVEENVPDGFVIPTELHASDPDTTA | |
| | | | ELRFSIDWDTSYATKQGRDADAKEFVNCIE | |
| | | | IETVYPNLNDRGTAIGRVVVREIREHVTID | |
| | | | YEMFEVLYLTVRVTDLNTVIGDDYDISTFT | |
| | | | IIIIDMNDNPPLWVEGTLTQEFRVREVAAS | |
| | | | GVVIGSVLATDIDGPLYNQVRYTITPRLDT | |
| | | | PEDLVEIDFNSGQISVKKHQAIDADEPPRQ | |
| | | | HLYCTVVASDKCDLLSVDVCPPDPNYFNTP | |
| | | | GEITIHITDTNNKVPRVEEDKFDETVYIYE | |
| | | | GAEDGEQVVQLFASDLDRDEIYHKVSYQTN | |
| | | | YAINPRLRDFFEVDLETGLVYVNNTAGEKL | |
| | | | DRDGDEPTHRIFFNVIDNFYGEGDGNRNQD | |
| | | | ETQVLVVLLDINDNYPELPEGLSWDISESL | |
| | | | LQGVRVTPDIFAPDRDEPGTDNSRVAYDIV | |
| | | | SLTPTDRDITLPQLFTMITIEKDRGIDQTG | |
| | | | ELETAMDLRGYWGTYEIHVKAYDHGVPQRI | |
| | | | SYEKYPLVIRPYNFHDPVFVFPQPGMTIRL | |
| | | | AKERAVVNGVLATVDGEFLERIVATDEDGL | |
| | | | HAGVVTFSISGDDEALQYFDVFNDGVNLGA | |
| | | | LTITQLFPEDFREFQVTIRATDGGTEPGPR | |
| | | | STDCTVTVVFVPTQGEPVFETSTYTVAFIE | |
| | | | KDAGMEERATLPLAKDPRNIMCEDDCHDTY | |
| | | | YSIVGGNSMGHFAVDPQSNELFLLTPLDRA | |
| | | | EQETHTLIIGASDSPSPAAVLQASTLTVTV | |
| | | | NVREANPRPVFQSALYTAGISTLDTINRAL | |
| | | | LTLHATHSEGLPVTYTLIQDSMEADSTLQA | |
| | | | VQETAFNLNPQTGVLTLNFQPTASMHGMFE | |
| | | | FDVMAIDTVGETARTEVKVYLISDRNRVFF | |
| | | | TFMNTLEEVEPNEDFMAETFTLFFGMRCNI | |
| | | | DQTLPASDPATGAARDDQTEVRAHFIRDDL | |
| | | | PVPAEEIEQLRGNPTLVATIQNALQEENLN | |
| | | | LADLFTGETPILGGEAQARAVYALAAVAAA | |
| | | | LALLCVVLLILFFIRTRALNRRLEALSMTK | |
| | | | YSSQDSGLNRVGLAAPGTNKHAVEGSNPIW | |
| | | | NETLKAPDFDALSEQSYDSDLIGIEDLPQF | |
| | | | RNDYFPPDEESSMRGVVNEHMPGANSVANH | |
| | | | NNNFGFNATPFSPEFANSQLRR | |
| 9 | Helicoverpa | DNA | ATGGCGACGAAACCCAAGACTCCTGGGTTC | Chitin |
| | armigera | | ACAGGTTTGGGAGATGACAGCGAAGACGAG | synthase |
| | | | TCGGAGTACACTCCGCTTTATGACGACGTC | B |
| | | | GACGATTTAGAACAAAGAACAGCTCAAGAA | |
| | | | ACAAAAGGATGGAATTTGTTCCGAGAACTC | |
| | | | CCCGTGAAGAAGGAGAGCGGGTCCATGGCG | |
| | | | TCCACAGCATGGATCGACACCAGTGTCAAG | |
| | | | ATCCTCAAGTTTCTGGCATACATCACCATA | |
| | | | TTTGTCGTCGTACTCGGATCTGCTGTTATT | |
| | | | TCTAAAGGCACTCTCCTTTTTATCACTTCT | |
| | | | CAACTTAAGAAGGGCAAACATATCACTCAT | |
| | | | TGTAACAGGGCATTAGCTTTAGATCAACAG | |
| | | | TTTATAACAGTCCACTCTTTGGAGGAGCGC | |
| | | | GTGACATGGCTATGGGCAGCCTTCATAATG | |
| | | | TTCAGTTTCCCGGAGGTGGGCGTATTCTTA | |
| | | | AGATCTGTCAGGATATGCTTCTTCAAAACC | |
| | | | GCTCTGAAGCCTACATTCCTTCACTTTCTT | |
| | | | GCGTCTATAGTAATAGAAACCCTGCACACT | |
| | | | GTTGGAATTGCGATGCTCGTTCTGATCATT | |
| | | | CTGCCCGAACTAGATGTCGTTAAAGGCACA | |
| | | | ATGTTGATGAATGCGATGTGCTTCGTGCCA | |
| | | | GGGCTCCTGAACGCCCTCTCGAGAGACMGA | |
| | | | AATGAGCGCCGATATGTTTGGAAGATCATG | |
| | | | TTAGACGTACTGGCGATCTCCGGCCAAGCT | |
| | | | ACTGCCTTCGTAGTCTGGCCTCTCCTTAAA | |
| | | | GGCGATACTATTCTATGGACTATTCCGGTA | |
| | | | GCTTGCGTGTTTGTTTCACTCGGCTGGTGG | |
| | | | GAAAACTTCGTCGGCAGCTCGGATCAACAA | |
| | | | TGGTCAGTCCTCCGACCTCTTCAAGARCTT | |
| | | | CGAGATGGTTTAAAAAGRACTCGTTATTAC | |
| | | | ACGCAGAGAGTTGTGTCWGTATGGAAGATA | |
| | | | TTTATATTCATGTGCTGCATTTTGATATCT | |
| | | | TTGGAAATACAACATGATGAYCCTTTTGCT | |
| | | | TTCTTCACAAAACTCACCACTGGTTTTGCC | |
| | | | GACCGCTTCTACATCGTACATGAGGTTCAA | |
| | | | GCAGTTCGAGATGAGTTCGAAGGCTTCTTG | |
| | | | GGCTACGCAGTGAAGGGATTAACGTTAGAA | |
| | | | ATACCAGCTTCATGGTCTACCCCACTATGG | |
| | | | GTGGTCCTCATCCAAGTGTTGGCCGCATAC | |
| | | | GTTTGTTTCGGAGCCAGTAAATTCGCCTGT | |
| | | | AAAATCCTCATTCAGAACTTCAGCTTTACA | |
| | | | TTTGCACTGAGCCTCGTAGGACCRGTCACT | |
| | | | ATTAACTTCTTGATTGCCGCATGCGGGATG | |
| | | | AGGAATGCGAACCCTTGTGCTTTTTACCGC | |
| | | | ACTATACCTGATTACTTGTTCTTCGATATT | |
| | | | CCACCGGTGTACTTCCTAAACGAGTTCGTA | |
| | | | GTACGCGAGATGTCRTGGGTWTGGTTGCTG | |
| | | | TGGATAGTCTCCCAAGCTTGGGTGACTGCT | |
| | | | CACACKTGGCAGCCGCGGTGTGAGCGACTC | |
| | | | GCKGCYACTGACAAACTGTTCGCCAAACCT | |
| | | | TGGTACTGCAGCGCGGTGATAGACCAGTCG | |
| | | | CTGTTGTTGAATAGGACCAAGGATGACGAC | |
| | | | ACTGATATAGCGTTAGAGGACCTCAAAGGT | |
| | | | TTGGATGCAGATGCTGATTCTATAGTTAGC | |
| | | | GGGGAAAAAGTTTCAAAGGATGTCAAGCCM | |
| | | | TCTGACAGTATAACAAGGATCTACGTRTGT | |
| | | | GCAACTATGTGGCACGAAACGAAAGAAGAA | |
| | | | ATGATGGAGTTCTTGAAATCTATTTTCCGT | |
| | | | CTCGACGAAGATCAGAGCGCTCGAAGGGTT | |
| | | | GCACAGAAGTACTTGGGCATTGTCGATCCT | |
| | | | GATTACTATGAACTAGAAGTGCACATTTTC | |
| | | | ATGGATGACGCGTTTGAGGTGTCCGATCAT | |
| | | | AGTTCGGAAGATTCGCAAGTGAATCGTTTC | |
| | | | GTCACGTGCCTCGTAGACACTATYGAYGAG | |
| | | | GCTGCYTCAGARGTCCAYCTCACAAACGTR | |
| | | | AGATTAAGACCCCCTAAGAAGTACCCCACC | |
| | | | CCATACGGCGGCCGACTYGTATGGACACTS | |
| | | | CCAGGAAAGAACAAAATGATTTGCCATCTM | |
| | | | AAAGACAAGTCCAAAATCAGACACAGGAAA | |
| | | | AGATGGTCTCAGGTGATGTACATGTACTAT | |
| | | | TTCCTTGGTCAYCGTTTGATGGACTTGCCR | |
| | | | ATCTCTGTGGATCGCAAGGAGGTTATCGCT | |
| | | | GAGAAYACTTATTTGTTGGCTYTGGATGGY | |
| | | | GACATTGACTTYAAGCCGATAGCCGTCACC | |
| | | | TTGCTGATTGATCTCATGAAGAAGGAYAAG | |
| | | | AACTTRGGAGCAGCGTGYGGACGTATCCAT | |
| | | | CCTGTGGGCTCTGGCTTCATGGCTTGGTAT | |
| | | | CAAATGTTCGAGTACGCTATTGGTCATTGG | |
| | | | CTGCAAAAGGCGACGGAACACATGATCGGC | |
| | | | TGCGTACTCTGTAGTCCTGGATGCTTCTCT | |
| | | | CTGTTCAGAGGAAAGGCTTTGATGGACGAC | |
| | | | AACGTCATGAAGAAATACACTTTGACTTCT | |
| | | | CACGAAGCCCGACATTACGTACAATATGAT | |
| | | | CAAGGTGAGGACCGTTGGCTGTGCACATTA | |
| | | | CTACTACAACGCGGCTACCGAGTCGAGTAC | |
| | | | TCTGCCGCGTCCGATGCGTACACGCACTGT | |
| | | | CCGGAACAATTCGACGAGTTCTTCAACCAG | |
| | | | CGACGACGATGGGTACCCTCTACTATGGCC | |
| | | | AACATATTCGATCTGCTGGCRGATGCTAAR | |
| | | | CGRACCATCTCGATAAATGATAATATTTCC | |
| | | | ACGCTTTATATTATGTATCAGTCTATGCTT | |
| | | | ATGTTCGGTACAATCCTCGGCCCCGGCACT | |
| | | | ATATTCCTGATGATGGTGGGAGCGATGAAC | |
| | | | GCCATCACTCAGATGAGCATGTCCAACGCG | |
| | | | CTCATACTCAACTTGGTGCCCATTCTCATA | |
| | | | TTCATCGTAGTCTGTATGACTTGCAAGTCT | |
| | | | GAAACGCAGCTATTCCTGGCGAGCTTGATA | |
| | | | ACATGCGCATACGCAATGGTGATGATGTTA | |
| | | | GTCATAGTGGGGATAGTCCTTCAAATCGTC | |
| | | | GAGGACGGATGGCTGGCCCCGTCCAGTTTG | |
| | | | TTCACGGCCGTCATATTCGGGACTTTCTTC | |
| | | | GTGACGGCRGCCCTTCATCCVCAGGAGATC | |
| | | | ATATGTTTGCTGTATCTAACTGTGTACTAT | |
| | | | GTGACCATTCCGAGTATGTACATGTTGCTC | |
| | | | ATTATATACTCGCTGTGCAATCTCAACAAC | |
| | | | GTGTCGTGGGGAACTAGGGAGGTGGTGCAG | |
| | | | AAGAAAACGGCTAAGGAAATGGAACAAGAA | |
| | | | CGCAAAGAAGCAGAAGAAGCTAAGAAGAAG | |
| | | | ATGGACGAGAAGAGCATACAGAAGTGGTTC | |
| | | | GGCAAGAGTGATGAGACCAGCGGCTCCTTG | |
| | | | GAGATGAGTGTGGCTGGTCTGTTCAAGTGT | |
| | | | ATGTGCTGCACCAATCCTAAGGACCACAAG | |
| | | | GAGGATCTGCATCTGCTGCAGATTGCTACA | |
| | | | GCCATCGAGAAGATTGATAAGAGATTGGAA | |
| | | | GCGCTCGGTGCACCTCCCGAAGAGACTGAG | |
| | | | CCGTTGAATCGTCGCCGGTCTTCAGCTGTA | |
| | | | TTACGTCGACAGTCGTTAGACCCGCTCGCC | |
| | | | AGAGTGCCGGAGTACGAAGAGAGCGATGTA | |
| | | | TCCAGCGACGTACCTAGGGACGAGCGTGAC | |
| | | | GATCTTATCAACCCGTACTGGATAGAAGAC | |
| | | | GTGAATCTTCAGAAGGGTGAAGTAGACTTC | |
| | | | CTGACCACGGCGGAGACTGAGTTCTGGAAG | |
| | | | GATCTGATCGATGTATACTTGAGGCCCATT | |
| | | | GATGAAAACAAACAGGAACTGGAACGTATC | |
| | | | AAAACGGACTTGAAGAATCTTCGCGACAAG | |
| | | | AGCGTGTTCGCGTTCGTAATGCTGAACTCT | |
| | | | TTGTTCGTGCTGATGATCTTCCTGCTGCAA | |
| | | | CTCAACCAGGATCAGCTGCACTTCAAGTGG | |
| | | | CCCTTCGGACAGTCAGCCAGTATAGAGTAC | |
| | | | GATGATCAGATGAATGTGTTCCACATAACA | |
| | | | CAAGACTACCTGACCCTGGAGCCGATCGGG | |
| | | | TCGCTGTTCATCATATTCTTCGGGTCCATC | |
| | | | ATCATCATCCAGTTCACCGCTATGCTGTTC | |
| | | | CATCGACTCGGCACGCTCACGCATCTGCTG | |
| | | | TCCAATGTTCAGCTCAACTGGTACTTCACT | |
| | | | AAGAAGCCGGACGACATGTCAGACAACGCC | |
| | | | CTAATAGAGTCTCGAGCACTAGAAATAGCC | |
| | | | AAAGACCTTCAACGCCTGAACACAGATGAC | |
| | | | CTAGAAAAGCGTGACAACAACCAACACGTG | |
| | | | AGCAGAAGGAAGACCATACATAACTTAGAG | |
| | | | AAAGGGAAGGATACTAAGCAGAGCGTTGTG | |
| | | | AATCTTGACGCCAACTTCAAGAGGAGGCTT | |
| | | | ACTATACTGCAAAATGGGGATGCTGAACTG | |
| | | | ATCTCCCGCCTACCATCCCTGGGAGGAACC | |
| | | | ACAGCGACGCGACGAGCTACTCTACGTGCT | |
| | | | CTCAAAACCAGACGCGACTCCGTGGTGGCC | |
| | | | GAGCGCCGCCGCTCACAGATGCAAGCCCGA | |
| | | | GACTCCACCACAGACTTCATGTTCAACTCG | |
| | | | CCCGGCGCGTTGGAGGATCTGGGCAGTCGG | |
| | | | GCGTCGGTTGGAGCGTACGTGAATCGTGGC | |
| | | | TACGAGCCTGCGCTCGATAGCGAAGTGGAG | |
| | | | GACACGCCGCCTCCTCCGAGAAGGTCCACC | |
| | | | GTGCGCTTCCAGGACCATTACGCGTGA | |
| 10 | Helicoverpa | Protein | MATKPKTPGFTGLGDDSEDESEYTPLYDDV | Chitin |
| | armigera | | DDLEQRTAQETKGWNLFRELPVKKESGSMA | synthase |
| | | | STAWIDTSVKILKFLAYITIFVVVLGSAVI | B |
| | | | SKGTLLFITSQLKKGKHITHCNRALALDQQ | |
| | | | FITVHSLEERVTWLWAAFIMFSFPEVGVFL | |
| | | | RSVRICFFKTALKPTFLHFLASIVIETLHT | |
| | | | VGIAMLVLIILPELDVVKGTMLMNAMCFVP | |
| | | | GLLNALSRDRNERRYVWKIMLDVLAISGQA | |
| | | | TAFVVWPLLKGDTILWTIPVACVFVSLGWW | |
| | | | ENFVGSSDQQWSVLRPLQELRDGLKRTRYY | |
| | | | TQRVVSVWKIFIFMCCILISLEIQHDDPFA | |
| | | | FFTKLTTGFADRFYIVHEVQAVRDEFEGFL | |
| | | | GYAVKGLTLEIPASWSTPLWVVLIQVLAAY | |
| | | | VCFGASKFACKILIQNFSFTFALSLVGPVT | |
| | | | INFLIAACGMRNANPCAFYRTIPDYLFFDI | |
| | | | PPVYFLNEFVVREMSWVWLLWIVSQAWVTA | |
| | | | HTWQPRCERLAATDKLFAKPWYCSAVIDQS | |
| | | | LLLNRTKDDDTDIALEDLKGLDADADSIVS | |
| | | | GEKVSKDVKPSDSITRIYVCATMWHETKEE | |
| | | | MMEFLKSIFRLDEDQSARRVAQKYLGIVDP | |
| | | | DYYELEVHIFMDDAFEVSDHSSEDSQVNRF | |
| | | | VTCLVDTIDEAASEVHLTNVRLRPPKKYPT | |
| | | | PYGGRLVWTLPGKNKMICHLKDKSKIRHRK | |
| | | | RWSQVMYMYYFLGHRLMDLPISVDRKEVIA | |
| | | | ENTYLLALDGDIDFKPIAVTLLIDLMKKDK | |
| | | | NLGAACGRIHPVGSGFMAWYQMFEYAIGHW | |
| | | | LQKATEHMIGCVLCSPGCFSLFRGKALMDD | |
| | | | NVMKKYTLTSHEARHYVQYDQGEDRWLCTL | |
| | | | LLQRGYRVEYSAASDAYTHCPEQFDEFFNQ | |
| | | | RRRWVPSTMANIFDLLADAKRTISINDNIS | |
| | | | TLYIMYQSMLMFGTILGPGTIFLMMVGAMN | |
| | | | AITQMSMSNALILNLVPILIFIVVCMTCKS | |
| | | | ETQLFLASLITCAYAMVMMLVIVGIVLQIV | |
| | | | EDGWLAPSSLFTAVIFGTFFVTAALHPQEI | |
| | | | ICLLYLTVYYVTIPSMYMLLIIYSLCNLNN | |
| | | | VSWGTREVVQKKTAKEMEQERKEAEEAKKK | |
| | | | MDEKSIQKWFGKSDETSGSLEMSVAGLFKC | |
| | | | MCCTNPKDHKEDLHLLQIATAIEKIDKRLE | |
| | | | ALGAPPEETEPLNRRRSSAVLRRQSLDPLA | |
| | | | RVPEYEESDVSSDVPRDERDDLINPYWIED | |
| | | | VNLQKGEVDFLTTAETEFWKDLIDVYLRPI | |
| | | | DENKQELERIKTDLKNLRDKSVFAFVMLNS | |
| | | | LFVLMIFLLQLNQDQLHFKWPFGQSASIEY | |
| | | | DDQMNVFHITQDYLTLEPIGSLFIIFFGSI | |
| | | | IIIQFTAMLFHRLGTLTHLLSNVQLNWYFT | |
| | | | KKPDDMSDNALIESRALEIAKDLQRLNTDD | |
| | | | LEKRDNNQHVSRRKTIHNLEKGKDTKQSVV | |
| | | | NLDANFKRRLTILQNGDAELISRLPSLGGT | |
| | | | TATRRATLRALKTRRDSVVAERRRSQMQAR | |
| | | | DSTTDFMFNSPGALEDLGSRASVGAYVNRG | |
| | | | YEPALDSEVEDTPPPPRRSTVRFQDHYA | |
| 11 | Spodoptera | DNA | ATGGCGAGACCAAGACCTTATGGTTTTAGG | Chitin |
| | frugiperda | | GCTTTAGATGAGGAGAGTGATGACAATTCG | synthase |
| | | | GAGTTGACTCCGTTGCACGATGATAATGAT | B |
| | | | GACCTAGGACAAAGAACAGCTCAAGAGGCA | |
| | | | AAAGGATGGAATCTGTTTCGAGAGATTCCG | |
| | | | GTGAAGAAGGAGAGTGGGTCTATGGCCTCA | |
| | | | ACTGCCGGGATAGACTTCAGTGTAAAGATC | |
| | | | CTTAAAGTCCTGGCGTATATTTTTATATTT | |
| | | | GGCATAGTGCTCGGATCTGCGGTTGTGTCT | |
| | | | AAGGGTACGCTGCTTTTTATCACATCACAA | |
| | | | CTGAAAAAGGGCAAAGCAATCGTTCACTGT | |
| | | | AATAGACAGTTAGAACTGGACAAGCAGTTT | |
| | | | ATAACAATCCATTCGTTGCAAGAGCGTGTG | |
| | | | ACGTGGCTATGGGCAGCCTTCATAGCATTC | |
| | | | AGTATTCCAGAAGTTGGCGTTTTCTTGAGA | |
| | | | TCAGTCAGAATATGCTTCTTCAAAACAGCA | |
| | | | CCGAAGCCTTCTGTTTTACAGTTTTTGACG | |
| | | | GCCTTCGTAGTAGACACCCTTCATACAATA | |
| | | | GGCATTGGATTACTGGTGCTTTTCATCCTG | |
| | | | CCAGAATTAGACGTGGTTAAAGGAACAATG | |
| | | | CTAATGAATGCTATGTGCTTCATGCCTGGA | |
| | | | ATACTAAACGCTGTGACCAGAGACCGCACG | |
| | | | GACTCTCGATACATGTTGAAAATGGCACTA | |
| | | | GATGTACTAGCTATCTCCGCTCAAGCCACC | |
| | | | GCGTTCGTCGTCTGGCCTCTGCTAAAAGGC | |
| | | | GTTAGTATGCTCTGGACGATTCCTGTCGCA | |
| | | | TGCGTATTCATCTCACTCGGATGGTGGGAA | |
| | | | AATTTCGTCGGCGATATCGGAAAACAATGG | |
| | | | CCAGTCCTGGAACCTGTACAAGAACTTCGT | |
| | | | GACAATTTAAAGAAGACTCGTTACTACACA | |
| | | | CAGAGGGTGTTGTCTTTGTGGAAGATATTC | |
| | | | ATATTCATGTGTTGCATCCTGATATCTTTG | |
| | | | GCGGCACAAGATGACAGCCCGCTTTCTTTC | |
| | | | TTCACGGAGTTTGCTACTGGATTTGGTGAG | |
| | | | CGCTTCTACAAAGTTCATGAGGTTCGAGCG | |
| | | | ATACAGGACGAATTTGAAGGTTTTCTGGGC | |
| | | | TACAAAATTATGGACTTATACTTCGATCAA | |
| | | | ATGCCAGCGGCATGGGCCACCCCACTGTGG | |
| | | | GTGGTGCTGATCCAGGTCCTGGCTTCTTTA | |
| | | | GTCTGTTTTATGGCAAGTTTGTCTGCCTGC | |
| | | | AAGATTCTGATACAAAACTTCAGCTTTACA | |
| | | | TTTGCGTTGAGTCTTGTTGGACCTGTCACC | |
| | | | ATCAACTTGTTGATTTGGCTTTGCGGCGAG | |
| | | | AGGAACGCAGATCCCTGCGCATATAGTAAT | |
| | | | ACGATACCAGATTATCTGTTCTTCGACATA | |
| | | | CCACCGGTGTATTTCCTGAAGGAGTTTGTG | |
| | | | GTGAAAGAGATGTCGTGGATTTGGTTGCTG | |
| | | | TGGCTGGTGTCGCAGGCGTGGGTGACGGCC | |
| | | | CACAACTGGCGCTCCCGGGCCGAGCGTCTC | |
| | | | GCCGCCAGCGACAAGCTCTTCAACAGGCCT | |
| | | | TGGTACTGCAGCCCCGTCCTCGACGTCTCC | |
| | | | ATGCTGTTGAACAGAACCAAGAATGAAGAA | |
| | | | GCGGAAATAACGATAGAGGATCTAAAAGAA | |
| | | | ACAGAGAGTGAGGGTGGGTCTATGATGAGC | |
| | | | GGATTTGAAGCAAAGAAAGACATAAAGCCT | |
| | | | TCTGACAACATTACGAGGATATATGTCTGC | |
| | | | GCGACTATGTGGCACGAAACGAAAGAAGAA | |
| | | | ATGATGGACTTCTTGAAGTCTATCCTGCGT | |
| | | | TTCGATGAGGATCAGAGCGCGCGTCGCGTC | |
| | | | GCACAGAAGTACTTGGGCATTGTAGATCCT | |
| | | | GATTACTATGAACTCGAAGTACACATCTTC | |
| | | | ATGGACGATGCTTTCGAAGTGTCGGACCAC | |
| | | | AGCGCGGACGACTCGAAAGTGAATCCCTTC | |
| | | | GTGACGTGTCTCGTGGAGACTGTCGACGAG | |
| | | | GCTGCTTCAGAGGTCCATCTCACCAACGTG | |
| | | | AGGTTGAGGCCACCGAAGAAATTCCCCACA | |
| | | | CCGTACGGCGGCCGACTGGTCTGGACTCTC | |
| | | | CCAGGAAAGAACAAAATGATATGCCACCTC | |
| | | | AAAGACAAGTCCAAAATACGACACAGGAAA | |
| | | | AGATGGTCTCAAGTGATGTACATGTACTAC | |
| | | | CTATTGGGCCACCGCCTGATGGACGTGCCG | |
| | | | ATCTCAGTGGACCGCAAGGAAGTCATCGCA | |
| | | | GGGAACACCTACTTACTGGCTTTGGACGGC | |
| | | | GACATTGACTTCAAACCGACAGCAGTCACG | |
| | | | TTACTAATCGATTTGATGAAGAAGGATAAG | |
| | | | AATTTAGGAGCAGCGTGCGGGCGCATCCAT | |
| | | | CCTGTGGGCTCAGGCTTCATGGCATGGTAT | |
| | | | CAAATGTTCGAGTACGCTATTGGTCATTGG | |
| | | | CTGCAAAAGGCGACTGAACACATGATTGGC | |
| | | | TGTGTACTCTGTAGCCCTGGATGCTTCTCC | |
| | | | CTCTTCAGAGGAAAGGCTTTGATGGACGAC | |
| | | | AACGTTATGAAGAAATATACCTTAACTTCC | |
| | | | CACGAGGCACGACACTATGTGCAATACGAT | |
| | | | CAAGGCGAGGACCGTTGGTGCACGCTACTG | |
| | | | CTGCAGCGCGGGTACCGCGTGGAGTACAGC | |
| | | | GCGGTGTCGGACGCGTACACGCACTGCCCC | |
| | | | GAGCACTTCGACGAGTTCTTCAACCAGCGC | |
| | | | CGCCGCTGGGTGCCCTCCACGCTCGCCAAC | |
| | | | ATCTTCGACCTGCTCGGCAGCGCCAAGCTC | |
| | | | ACCGTCAAGTCCAACGACAACATCTCCACC | |
| | | | CTCTATATAGTCTATCAGTTCATGTTGATA | |
| | | | GTGGGTACGGTGTTGGGTCCCGGCACGATC | |
| | | | TTCCTGATGATGGGGGGAGCCATGAACGCC | |
| | | | ATCATTCAGATCAGCAACGCGTACGCGATG | |
| | | | ATGTTGAACCTCGTACCACTCGTCATCTTC | |
| | | | CTTATAGTCTGTATGACTTGTCAGTCAAAG | |
| | | | ACGCAGCTCTTCCTCGCTAACCTCATAACA | |
| | | | TGCGCATACGCAATGGTGATGATGATCGTG | |
| | | | ATAGTGGGGATAGTTCTGCAGATAGTGGAG | |
| | | | GATGGATGGCTGGCTCCGTCCAGTATGTTC | |
| | | | ACAGCTTTAATATTCGGTACATTCTTCGTC | |
| | | | ACCGCGGCACTACACCCGCAAGAGATCAAA | |
| | | | TGTTTGTTGTTCATAGCAGTGTACTATGTA | |
| | | | ACCATCCCTAGTATGTACATGTTGTTGATC | |
| | | | ATATACTCCATCTGTAATCTCAACAACGTA | |
| | | | TCCTGGGGTACCAGGGAGACACCGCAGAAG | |
| | | | AAAACTGCTAAGGAAATGGAAATGGAACAG | |
| | | | AAGGAAGCAGAAGAAGCGAAGAAAAAAATG | |
| | | | GAGAGTCAGGGTTTGAAGAAGTTGTTTGCC | |
| | | | AAGGGAGAAGAGAAGAGTGGTTCGTTAGAG | |
| | | | TTCAGTGTGGCGGGCCTGTTGCGATGTATG | |
| | | | TGCTGCACCAATCCAGAGGATCATAAGGAC | |
| | | | GATCTCAACATGATGCAGATCTCACACGCG | |
| | | | TTGGAGAAGATAAATAAGAGATTGGATCAA | |
| | | | CTCGATGTCCCTCCTGAGCCGACCCACCAG | |
| | | | CCCTCGCATCCGCACACACACGTGGAGACG | |
| | | | GTCGGTGTTCGTGATTACGAAGACAGCGAG | |
| | | | ATTTCCACTGAAATTCCTAAGGAAGAACGA | |
| | | | GACGACCTGATTAACCCCTACTGGATCGAG | |
| | | | GACGTGGAACTCCAGAAGGGCGAGGTAGAC | |
| | | | TTCCTCACCACCGCTGAGACCAACTTCTGG | |
| | | | AAGGATGTCATCGATGAATACTTACTGCCT | |
| | | | ATTGATGAGGACAAGCGTGAAATTGAACGT | |
| | | | ATAAGAAAAGATTTGAAGAACTTGCGAGAT | |
| | | | AAGATGGTGTTTGCGTTCGTGATGTTGAAC | |
| | | | TCTCTGTTCGTGCTCGTCATCTTCCTGCTG | |
| | | | CAGCTCAGCCAGGACCAGCTGCACTTCAAG | |
| | | | TGGCCATTCGGACAGAAGTCCAGCATGGAG | |
| | | | TACGATAATGATATGAATATGTTCATCATA | |
| | | | ACCCAAGACTACTTAACGCTGGAACCTATC | |
| | | | GGTTTCGTGTTCCTCCTGTTCTTCGGCTCC | |
| | | | ATCATCATGATCCAGTTCACCGCCATGTTG | |
| | | | TTCCATCGCCTGGACACGCTGGCCCATCTG | |
| | | | CTGTCCACCACCAAGCTGGATTGGTATTTC | |
| | | | AGTAAGAAGCCGGACGACCTATCAGACGAT | |
| | | | GCGCTAATAGACTCTTGGGCGTTGACAATA | |
| | | | GCGAAGGATCTTCAACGTCTGAACACCGAC | |
| | | | GACTTGGATAAACGAAATAACAACGAACAC | |
| | | | GTGTCCAGGAGGAAGACCATATATAACTTG | |
| | | | GAGAAAGGGAAGGAAACCAAACCGGCTGTT | |
| | | | ATCAACCTCGATGCCAACGCCAAGAGGAGA | |
| | | | TTGACTATCCTGCAGAATGAAGACTCAGAA | |
| | | | TTGATCTCCCGCCTGCCATCTCTGGGACCT | |
| | | | AATTTGGCAACTCGTCGTGCCACGGTGCGT | |
| | | | GCAATAAACACTCGACGCGCATCTGTCATG | |
| | | | GCGGAGCGACGCAGGTCTCAGTTCCAAGCG | |
| | | | CGACCTTCCGGGGGATCATACATGTATAAT | |
| | | | AACCCTCAAAACACGATTCAGCTGGACGAT | |
| | | | ATGGTCGGGGGGCCGTCGACGTCGGGAGTG | |
| | | | TACGTGAACCGAGGGTACGAGCCCGCCCTG | |
| | | | GACAGCGACATCGAGGACACGCCCGTGCCC | |
| | | | ACCAGACGATCCGTTGTACACTTCACCGAC | |
| | | | CATTTCGCGTGA | |
| 12 | Spodoptera | Protein | MARPRPYGFRALDEESDDNSELTPLHDDND | Chitin |
| | frugiperda | | DLGQRTAQEAKGWNLFREIPVKKESGSMAS | synthase |
| | | | TAGIDFSVKILKVLAYIFIFGIVLGSAVVS | B |
| | | | KGTLLFITSQLKKGKAIVHCNRQLELDKQF | |
| | | | ITIHSLQERVTWLWAAFIAFSIPEVGVFLR | |
| | | | SVRICFFKTAPKPSVLQFLTAFVVDTLHTI | |
| | | | GIGLLVLFILPELDVVKGTMLMNAMCFMPG | |
| | | | ILNAVTRDRTDSRYMLKMALDVLAISAQAT | |
| | | | AFVVWPLLKGVSMLWTIPVACVFISLGWWE | |
| | | | NFVGDIGKQWPVLEPVQELRDNLKKTRYYT | |
| | | | QRVLSLWKIFIFMCCILISLAAQDDSPLSF | |
| | | | FTEFATGFGERFYKVHEVRAIQDEFEGFLG | |
| | | | YKIMDLYFDQMPAAWATPLWVVLIQVLASL | |
| | | | VCFMASLSACKILIQNFSFTFALSLVGPVT | |
| | | | INLLIWLCGERNADPCAYSNTIPDYLFFDI | |
| | | | PPVYFLKEFVVKEMSWIWLLWLVSQAWVTA | |
| | | | HNWRSRAERLAASDKLFNRPWYCSPVLDVS | |
| | | | MLLNRTKNEEAEITIEDLKETESEGGSMMS | |
| | | | GFEAKKDIKPSDNITRIYVCATMWHETKEE | |
| | | | MMDFLKSILRFDEDQSARRVAQKYLGIVDP | |
| | | | DYYELEVHIFMDDAFEVSDHSADDSKVNPF | |
| | | | VTCLVETVDEAASEVHLTNVRLRPPKKFPT | |
| | | | PYGGRLVWTLPGKNKMICHLKDKSKIRHRK | |
| | | | RWSQVMYMYYLLGHRLMDVPISVDRKEVIA | |
| | | | GNTYLLALDGDIDFKPTAVTLLIDLMKKDK | |
| | | | NLGAACGRIHPVGSGFMAWYQMFEYAIGHW | |
| | | | LQKATEHMIGCVLCSPGCFSLFRGKALMDD | |
| | | | NVMKKYTLTSHEARHYVQYDQGEDRWCTLL | |
| | | | LQRGYRVEYSAVSDAYTHCPEHFDEFFNQR | |
| | | | RRWVPSTLANIFDLLGSAKLTVKSNDNIST | |
| | | | LYIVYQFMLIVGTVLGPGTIFLMMGGAMNA | |
| | | | IIQISNAYAMMLNLVPLVIFLIVCMTCQSK | |
| | | | TQLFLANLITCAYAMVMMIVIVGIVLQIVE | |
| | | | DGWLAPSSMFTALIFGTFFVTAALHPQEIK | |
| | | | CLLFIAVYYVTIPSMYMLLIIYSICNLNNV | |
| | | | SWGTRETPQKKTAKEMEMEQKEAEEAKKKM | |
| | | | ESQGLKKLFAKGEEKSGSLEFSVAGLLRCM | |
| | | | CCTNPEDHKDDLNMMQISHALEKINKRLDQ | |
| | | | LDVPPEPTHQPSHPHTHVETVGVRDYEDSE | |
| | | | ISTEIPKEERDDLINPYWIEDVELQKGEVD | |
| | | | FLTTAETNFWKDVIDEYLLPIDEDKREIER | |
| | | | IRKDLKNLRDKMVFAFVMLNSLFVLVIFLL | |
| | | | QLSQDQLHFKWPFGQKSSMEYDNDMNMFII | |
| | | | TQDYLTLEPIGFVFLLFFGSIIMIQFTAML | |
| | | | FHRLDTLAHLLSTTKLDWYFSKKPDDLSDD | |
| | | | ALIDSWALTIAKDLQRLNTDDLDKRNNNEH | |
| | | | VSRRKTIYNLEKGKETKPAVINLDANAKRR | |
| | | | LTILQNEDSELISRLPSLGPNLATRRATVR | |
| | | | AINTRRASVMAERRRSQFQARPSGGSYMYN | |
| | | | NPQNTIQLDDMVGGPSTSGVYVNRGYEPAL | |
| | | | DSDIEDTPVPTRRSVVHFTDHFA | |
| 13 | Helicoverpa | DNA | ATGGGTGCCAAAATGTTGCTTCCCACCGTA | Amino |
| | armigera | | TTCTGCATCCTCCTGGGATCCATAGCGGCC | peptidase N |
| | | | ATTCCTCAAGAGGACTTCAGGTCCAACTTG | |
| | | | GAGTGGTCTGACTACAGCACCAACTTAGAC | |
| | | | GAGCCGGCGTACCGTCTGCGTGATGTGGTC | |
| | | | TATCCTACTGATGTCAACCTGGATCTGGAT | |
| | | | GTCTACCTAAACCACCTGAACTTCTCTGGA | |
| | | | CTTGTACAGATTGATGTTCAAGTACGAGAG | |
| | | | AACAATTTACGCCAAATTGTTCTTCACCAA | |
| | | | AAGGTGGTTTCCATCACTGGAGTGAATGTT | |
| | | | GTCGGACCTAACGGCCCAGTTCCTCTCCAG | |
| | | | TTCCCCCACCCTTATACCACTGATGATTAC | |
| | | | TATGAGATCCTTCTCATCAACTTGGACCAG | |
| | | | CCCATCAACATTGGCAACTACTCCATCGCC | |
| | | | ATCAGATACAATGGCCAGATCAATGCTAAC | |
| | | | CCTCTTGACCGAGGTTTTTACAGAGGCTAC | |
| | | | TATCACCTGAACAATGAATTGAGGGTCTAC | |
| | | | GCCACCACCCAGTTCCAGCCTTACCACGCC | |
| | | | AGGAAGGCCTTCCCTTGCTTCGATGAACCC | |
| | | | CAATTCAAATCCCGCTACACTATCTCCATT | |
| | | | ACTCGCGACACCAGTCTGTCTCCATCGTAC | |
| | | | TCCAACATGGCTATTAGAAGCGCCCAAGAC | |
| | | | ATTAGTACCTCCCGTATTCGCGAAAACTTC | |
| | | | TACACTACGCCCATCATCTCCGCCTATCTG | |
| | | | GTCGCTTTCCACGTCAGTGACTTCGTCTCC | |
| | | | ACTGAATACACCAGCACCGATGCCAAACCC | |
| | | | TTCAGTATTATCTCCCGCCAAGGTGCCACG | |
| | | | AACCAGCACCAATATGCTGCTGAAATCGGT | |
| | | | CTTAAGATCACCAACGAACTCGATGACTAC | |
| | | | TTTGGCATCCAGTACCATGAGATGGGACAA | |
| | | | GGTGCTTTGATGAAGAATGACCACATCGCT | |
| | | | CTTCCTGACTTCCCCTCCGGTGCTATGGAG | |
| | | | AACTGGGGAATGGTTAACTACAGGGAAGCC | |
| | | | TACCTCTTGTACGACGAAAACAACACCAAC | |
| | | | TTGAACAACAAGATTTTCATCGCTACCATC | |
| | | | ATGGCTCACGAATTGGGACACAAATGGTTC | |
| | | | GGTAACCTCGTCACTTGCTTCTGGTGGAGC | |
| | | | AACCTTTGGCTTAACGAGTCTTTCGCCAGC | |
| | | | TTCTTCGAATACTTCGGCGCTCACTGGGCT | |
| | | | GATCCCGCTCTAGAGTTAGATGACCAGTTT | |
| | | | GTCGTTGACTACGTGCACAGCGCTCTCAAC | |
| | | | TCTGACGCCAGCCAGTACGCCACTCCCATG | |
| | | | AACCACACCGACGTCGTGGACAATGACTCC | |
| | | | ATCACCTCCCACTTCAGTGTTACCAGCTAC | |
| | | | GCTAAGGGAGCTTCCGTCCTTAAGATGATG | |
| | | | GAACATTTCGTTGGATGGAGGACCTTCAGA | |
| | | | AACGCTCTCAGATACTACTTGAGAAACAAC | |
| | | | GAGTACGACATCGGTTTCCCCGTTGATATG | |
| | | | TACACGGCTTTCAAGCAAGCAGTCGCTGAA | |
| | | | GATTTTACTTTCCAACGTGATTTCCCTAAT | |
| | | | GTTGACGTTGGCGCAGTATTCGACAGCTGG | |
| | | | GTCCAGAACCCTGGCTCTCCCGTCATCAAC | |
| | | | GTTGCCCGTAACAATAACACAGGTGTCATC | |
| | | | ACTGTCAACCAGCAACGTTACGTGCTCTCC | |
| | | | GGCGCTGTTGCCTCAACGACGTGGCACATT | |
| | | | CCTCTCACCTGGACTCAACATGGCTCCCTC | |
| | | | AACTTCAACAGCACCAGGCCTAGCACCGTC | |
| | | | CTTAGCGATGAGATTGGCACCATCAACGCT | |
| | | | GCATCTGGAGACCACTTCGTCATTTTCAAC | |
| | | | ATTGCCCAATCTGGTCTGTACCGTGTCAAC | |
| | | | TACGACACCAACAACTGGCAGTTGCTTGCT | |
| | | | TCATACCTGAAGAGCAACAACAGACAGAAC | |
| | | | ATTCACAAGCTGAACAGAGCTCAGATCGTC | |
| | | | AACGACATCTTGTACTTCGTGCGTTCTAAC | |
| | | | AGCATCAACAGGACTCTCGCTTTTGATGTC | |
| | | | CTCGACTTCTTGAGGGATGAGACCGATTAC | |
| | | | TACGTATGGAATGGAGCTCTTACCCAGATC | |
| | | | GACTGGATCCTTCGTCGCCTTGAACACTTG | |
| | | | CCTACCGCTCATGCTGCTTTCTCTGAATAC | |
| | | | ATCCTCGAGCTCATGAACACGGTTATCAAT | |
| | | | CACCTTGGCTATAACGAGCACAGTACCGAC | |
| | | | TCTACCTCCACAATCCTCAACCGTATGCAG | |
| | | | ATCATGAACTACGCTTGCAACCTTGGACAC | |
| | | | AGTGGCTGCATTTCTGACAGTTTAGACAAA | |
| | | | TGGAGGCAGCACCGTGCTAACGTATCTAAC | |
| | | | TTGGTACCAGTAAATCTCCGTCGTTACGTT | |
| | | | TACTGCGTTGGTCTTCGTGAGGGTAACGAA | |
| | | | ACTGACTACAACTACCTGTATAGCGTGTAC | |
| | | | AATTCTTCAGAGAACACTGCTGACATGGTT | |
| | | | GTTATCCTCCGCGCCCTCGCTTGCACCAAG | |
| | | | CATCAGCCTTCTCTTGAGCACTACTTGCAA | |
| | | | CAGTCCATGTACAACGACAAAGTTCGTATC | |
| | | | CACGACCGCACCAACGCGTTCTCCTTCGCT | |
| | | | CTGCAAGGCAACCCTGAGAACCTTCCCATC | |
| | | | GTTCTGAACTTCCTCTACAACAACTTTGCC | |
| | | | GCTATCAGGGAAACGTACGGAGGTGTTGCC | |
| | | | CGTCTCAATATTTGCCTCAACGCTATTGCG | |
| | | | GCATTCTTGACTGACTACCAGACCATCACT | |
| | | | CAGTTCCAAACTTGGGTGTACTCCAACCAA | |
| | | | TTGGAGCTGGTTGGCTCTGTCGGCGTTGGA | |
| | | | AATAACGTCGTCGCCGCCGCCTTGAACAAT | |
| | | | CTCACTTGGGGCAACGGCGCAGCTGTTGAA | |
| | | | ATTGTCAACTTCCTCAACTCTAGAAGCGGT | |
| | | | TCCACCACCATCCTTGCTTCTTCAATCCTC | |
| | | | ATCTTAGCAGCCATGCTTCTACAAATGTTC | |
| | | | CGCTAAGATGTC | |
| 14 | Helicoverpa | Protein | MGAKMLLPTVFCILLGSIAAIPQEDFRSNL | Amino |
| | armigera | | EWSDYSTNLDEPAYRLRDVVYPTDVNLDLD | peptidase |
| | | | VYLNHLNFSGLVQIDVQVRENNLRQIVLHQ | |
| | | | KVVSITGVNVVGPNGPVPLQFPHPYTTDDY | |
| | | | YEILLINLDQPINIGNYSIAIRYNGQINAN | |
| | | | PLDRGFYRGYYHLNNELRVYATTQFQPYHA | |
| | | | RKAFPCFDEPQFKSRYTISITRDTSLSPSY | |
| | | | SNMAIRSAQDISTSRIRENFYTTPIISAYL | |
| | | | VAFHVSDFVSTEYTSTDAKPFSIISRQGAT | |
| | | | NQHQYAAEIGLKITNELDDYFGIQYHEMGQ | |
| | | | GALMKNDHIALPDFPSGAMENWGMVNYREA | |
| | | | YLLYDENNTNLNNKIFIATIMAHELGHKWF | |
| | | | GNLVTCFWWSNLWLNESFASFFEYFGAHWA | |
| | | | DPALELDDQFVVDYVHSALNSDASQYATPM | |
| | | | NHTDVVDNDSITSHFSVTSYAKGASVLKMM | |
| | | | EHFVGWRTFRNALRYYLRNNEYDIGFPVDM | |
| | | | YTAFKQAVAEDFTFQRDFPNVDVGAVFDSW | |
| | | | VQNPGSPVINVARNNNTGVITVNQQRYVLS | |
| | | | GAVASTTWHIPLTWTQHGSLNFNSTRPSTV | |
| | | | LSDEIGTINAASGDHFVIFNIAQSGLYRVN | |
| | | | YDTNNWQLLASYLKSNNRQNIHKLNRAQIV | |
| | | | NDILYFVRSNSINRTLAFDVLDFLRDETDY | |
| | | | YVWNGALTQIDWILRRLEHLPTAHAAFSEY | |
| | | | ILELMNTVINHLGYNEHSTDSTSTILNRMQ | |
| | | | IMNYACNLGHSGCISDSLDKWRQHRANVSN | |
| | | | LVPVNLRRYVYCVGLREGNETDYNYLYSVY | |
| | | | NSSENTADMVVILRALACTKHQPSLEHYLQ | |
| | | | QSMYNDKVRIHDRTNAFSFALQGNPENLPI | |
| | | | VLNFLYNNFAAIRETYGGVARLNICLNAIA | |
| | | | AFLTDYQTITQFQTWVYSNQLELVGSVGVG | |
| | | | NNVVAAALNNLTWGNGAAVEIVNFLNSRSG | |
| | | | STTILASSILILAAMLLQMFR | |
| 15 | Heliothis | DNA | ATGGGTGCCAAAATGTTGCTTCCCACCGTG | Amino |
| | virescens | | TTTTGTATCCTTCTGGGATCCATAGCAGCC | peptidase |
| | | | ATTCCTCAAGAGGACTTCAGGTCGAACTTG | |
| | | | GAGTGGGCTGACTACAGCACCAATTTAGAC | |
| | | | GAGCCGGCGTACCGTCTCCGTGATGTGGTG | |
| | | | TACCCTACTGATGTCAACCTGGATCTGGAC | |
| | | | GTGTACCTCGATGAACTAAGGTTCAATGGA | |
| | | | TTGGTACAGATTGATGTCGAAGTACGAGAG | |
| | | | AACGATTTACGTCAAATTGTTCTTCATCAG | |
| | | | AAGGTCGTCTCAATCAACGCTGTTAACGTT | |
| | | | GTCGGACCCAACGGTCCAGTCGGTCTACAG | |
| | | | TTCCCATACCCTTACACCACTGATGATTAC | |
| | | | TACGAGATCCTCCTCATCAACCTGGCTGAG | |
| | | | CCCATCAACATCGGCAACTACTCCATCACC | |
| | | | ATCAGATACAACGGCCAGATCAATGATAAC | |
| | | | CCTATCGACAGAGGTTTCTACAAAGGCTAC | |
| | | | TATTACCTGAACAATGAATTGAGGCTCTAC | |
| | | | GCCACCACACAGTTCCAGCCTTACCACGCC | |
| | | | AGGAAGGCCTTCCCGTGCTTCGACGAGCCC | |
| | | | CAATTCAAATCCCGCTTCACTATCTCCATC | |
| | | | ACTCGTGCCAGCAGCCTGTCTCCTTCATAC | |
| | | | TCCAACATGGCTATCAGCAACACTCAAATC | |
| | | | CTTGGTGCCCGTACTCGCGAAACTTTCCAT | |
| | | | CCTACGCCCATCATCTCCGCCTACCTGGTT | |
| | | | GCTTTCCACGTCAGTGACTTCGTCGCCACT | |
| | | | GAATACACCAGCACCGATGCCAAACCCTTC | |
| | | | AGTATTATCTCCCGCCAAGGTGTCACAGAC | |
| | | | CAGCACGAATATGCCGCTGAGATCGGTCTC | |
| | | | AAGATCACCAACGAGCTTGATGACTACTTA | |
| | | | GGCATCCAGTACCATGAGATGGGACAAGGT | |
| | | | ACCCTGATGAAGAATGACCACATTGCTCTT | |
| | | | CCTGACTTCCCCTCCGGTGCTATGGAGAAC | |
| | | | TGGGGAATGGTAAACTACAGGGAGGCTTAC | |
| | | | CTTTTGTACGACGCTAACAACACCAACTTA | |
| | | | AACAATAAGATTTTCATCGCTACCATCATG | |
| | | | GCTCACGAGCTGGGACACAAATGGTTCGGT | |
| | | | AACCTCGTCACCTGCTTCTGGTGGAGCAAC | |
| | | | CTTTGGCTAAACGAGTCTTTCGCCAGCTTT | |
| | | | TTCGAATACCTTGGTGCTCACTGGGCTGAT | |
| | | | CCCGCTCTAGAGTTAGATGACCAGTTTGTC | |
| | | | GTCGACTACGTACACAGCGCTCTCAATTCT | |
| | | | GACGCCAGTCAATTCGCCACCCCTATGAAC | |
| | | | CATGTCGACGTTGTGGACAACGACTCAATC | |
| | | | ACCGCCCACTTCAGTGTCACTAGCTACGCT | |
| | | | AAGGGCGCTTCCGTCCTTAGGATGATGGAA | |
| | | | CACTTCGTTGGATCGAGGACCTTCAGAAAT | |
| | | | GCCCTCAGATATTACTTGAGAAACAACGAG | |
| | | | TATAGTATAGGTTTCCCCGTTGATATGTAC | |
| | | | GCGGCTTTCAAGCAAGCCGTCTCTGAAGAT | |
| | | | TTTACCTTCGAACGTGATTTCCCCGGTATT | |
| | | | GACGTTGGAGCAGTATTCGACACCTGGGTC | |
| | | | CAGAACCGTGGATCTCCCGTCTTGAACGTT | |
| | | | GCCCGTAACAGCAACACTGGTGTCATCAGT | |
| | | | GTCAGCCAGGAACGCTATGTGCTCTCGGGC | |
| | | | GCTGTAGCTCCAGCGTTGTGGCAGATTCCT | |
| | | | CTCACCTTGACTCAAAATGGCTCCCTCAAC | |
| | | | TTTGAGAACACCAGACCTAGCTTGGTCCTT | |
| | | | ACTACCCAGAGCCAGAATATCAACGGTGCC | |
| | | | TCTGGAGATAACTTTGTTATTTTCAACAAT | |
| | | | GCTCAGTCCGGTCTGTACCGTGTTAACTAC | |
| | | | GACACAAACAACTGGCAGTTGCTTGCTTCA | |
| | | | TACCTGAAGAGCAACAACAGAGAGAACATT | |
| | | | CACAAGCTGAACAGAGCCCAGATCGTCAAC | |
| | | | GATGTCTTGAACTTCGTGCGTTCCAACAGC | |
| | | | ATCAACAGAACCCTCGCTTTTGAAGTTCTC | |
| | | | GACTTCTTGAGAGATGAGACCGATTACTAT | |
| | | | GTATGGAACGGAGCTCTTACCCAGATCGAC | |
| | | | TGGATCCTTCGTCGCCTTGAGCATTTGCCG | |
| | | | GCTGCCCATGCTGCTTTCTCTGAATACATC | |
| | | | CTTGATCTCATGAGCACGGTCATCAACCAC | |
| | | | CTTGGTTACAATGAGCAGAGTACTGACTCC | |
| | | | ACCTCCACAATCCTCAACCGAATGCAGATC | |
| | | | ATGAACTATGCCTGCAATCTTGGACACAGT | |
| | | | GGTTGCATTGCTGACAGTTTAGACAAATGG | |
| | | | AGGCAGCACCGTGAGAACCCGAATAACTTG | |
| | | | GTGCCAGTGAATCTCCGTCGTTACGTGTAC | |
| | | | TGCGTCGGTCTGCGTGAAGGCAACGAAACT | |
| | | | GACTACAGCTACTTGTTCAGCGTGTACAAT | |
| | | | TCCTCAGAGAACACCGCTGACATGGTTGTG | |
| | | | ATACTCCGCGCTCTCGCCTGCACCAAACAC | |
| | | | CAGCCATCTCTTGAGCACTATCTGCAAGAG | |
| | | | TCCATGTACAACGACAAAATCCGTATCCAC | |
| | | | GACCGTACAAATGCATTCTCCTTCGCTCTG | |
| | | | CAAGGCAACCCTGAGAACCTTCCCATCGTC | |
| | | | CTTAACTTCCTCTACAACAACTTTGCCGCT | |
| | | | ATCAGGGAAACGTATGGAGGTGTGGCCCGT | |
| | | | CTCAATCTGTGCATCAACGCAATCCCTGCA | |
| | | | TTCTTGACTGACTACCAGACTATTACTCAG | |
| | | | TTCCAATCTTGGGTATACGCAAACCAATTG | |
| | | | GCGTTGGTTGGTTCATTCAACAATGGCGTT | |
| | | | AGCGTCGTCAACACCGCCTTGGATAACCTT | |
| | | | ACTTGGGGCAACGGTGCTGCTGTTGAGATC | |
| | | | GTCAATTTCCTCAACTACAAGAGTGCATCC | |
| | | | CCCTCCATCCTTGCTTCTTCCATCCTCATC | |
| | | | TTAGCAGCTATGCTCGTACAAATGTTCCGC | |
| | | | TAAGGTGTCTCATTCTAAGTCGTTACACTT | |
| | | | CACATAATTCTAATTTAAGTTTATCTATTT | |
| | | | TGTTTTATACAATCGTTCCGTCGTTTTGTT | |
| | | | TATGGTTGTGAAGTATTTATTGTAGATTTA | |
| | | | TTGATAATAAATGTTCTTTTGTAAAGAAAA | |
| | | | AAAAAAAAAAAAAA | |
| 16 | Heliothis | Protein | MGAKMLLPTVFCILLGSIAAIPQEDFRSNL | Amino |
| | virescens | | EWADYSTNLDEPAYRLRDVVYPTDVNLDLD | peptidase N |
| | | | VYLDELRFNGLVQIDVEVRENDLRQIVLHQ | |
| | | | KVVSINAVNVVGPNGPVGLQFPYPYTTDDY | |
| | | | YEILLINLAEPINIGNYSITIRYNGQINDN | |
| | | | PIDRGFYKGYYYLNNELRLYATTQFQPYHA | |
| | | | RKAFPCFDEPQFKSRFTISITRASSLSPSY | |
| | | | SNMAISNTQILGARTRETFHPTPIISAYLV | |
| | | | AFHVSDFVATEYTSTDAKPFSIISRQGVTD | |
| | | | QHEYAAEIGLKITNELDDYLGIQYHEMGQG | |
| | | | TLMKNDHIALPDFPSGAMENWGMVNYREAY | |
| | | | LLYDANNTNLNNKIFIATIMAHELGHKWFG | |
| | | | NLVTCFWWSNLWLNESFASFFEYLGAHWAD | |
| | | | PALELDDQFVVDYVHSALNSDASQFATPMN | |
| | | | HVDVVDNDSITAHFSVTSYAKGASVLRMME | |
| | | | HFVGSRTFRNALRYYLRNNEYSIGFPVDMY | |
| | | | AAFKQAVSEDFTFERDFPGIDVGAVFDTWV | |
| | | | QNRGSPVLNVARNSNTGVISVSQERYVLSG | |
| | | | AVAPALWQIPLTLTQNGSLNFENTRPSLVL | |
| | | | TTQSQNINGASGDNFVIFNNAQSGLYRVNY | |
| | | | DTNNWQLLASYLKSNNRENIHKLNRAQIVN | |
| | | | DVLNFVRSNSINRTLAFEVLDFLRDETDYY | |
| | | | VWNGALTQIDWILRRLEHLPAAHAAFSEYI | |
| | | | LDLMSTVINHLGYNEQSTDSTSTILNRMQI | |
| | | | MNYACNLGHSGCIADSLDKWRQHRENPNNL | |
| | | | VPVNLRRYVYCVGLREGNETDYSYLFSVYN | |
| | | | SSENTADMVVILRALACTKHQPSLEHYLQE | |
| | | | SMYNDKIRIHDRTNAFSFALQGNPENLPIV | |
| | | | LNFLYNNFAAIRETYGGVARLNLCINAPAF | |
| | | | LTDYQTITQFQSWVYANQLALVGSFNNGVS | |
| | | | VVNTALDNLTWGNGAAVEIVNFLNYKSASP | |
| | | | SILASSILILAAMLVQMFR | |
| 17 | Helicoverpa | DNA | AGCGACTTTACTGGTTCAGTTAGAATCGCG | Alkaline |
| | armigVera | | ATGGTGACACTGTTCCCGTACGTAGTGGCG | phosphatase |
| | | | GTGCTGTGCGGCGCGACGAGCGCGCGCGCC | |
| | | | CACTGGCTGCATCCCGCGGCGCCGGCCGCC | |
| | | | GCCAGCCGCGCCGAGACCTCTGCCAACTAC | |
| | | | TGGGCGCAAGACGCGCAGGCCGCCATCAAC | |
| | | | GCTCGCCTGGAACGAGTTGAAAGCGTGAAG | |
| | | | AAAGCGCGTAACGTCATCATGTTCCTGGGC | |
| | | | GACGGCATGTCGGTGCCCACGCTCGCCGCC | |
| | | | GCGCGCACGCTGCTCGGGCAGCGCCAAGGG | |
| | | | AAAACGGGAGAGGAGACAAAGTTGCATTTC | |
| | | | GAGACTTTCCCCACAATCGGATTAGTGAAG | |
| | | | ACGTACTGTGTGGACGCCCAGATTGCAGAC | |
| | | | TCCGCATGTACTGCCACAGCGTATTTGTGT | |
| | | | GGTGTAAAAAATAACTATGGCGCCATAGGC | |
| | | | GTAGACGGCACGGTACGCCGAGGAGACTGT | |
| | | | CAAGCCGCTTCAAACACTGCGACACACGTC | |
| | | | GAGTCCATCGCGGAGTGGGCGCTCGCTGAC | |
| | | | GGACGAGATGTCGGTATTGTGACGACGACT | |
| | | | CGTATCACTCACGCGTCTCCGGCGGGCACG | |
| | | | TTCGCGAAGACGGCGAACCGCACCTGGGAG | |
| | | | AACGACGGTGAAGTGTCGCAGATGGGCTTG | |
| | | | GACGCCAAGGACTGCCCTGACATCGCGCAT | |
| | | | CAGTTGGTACACCATCATCCCGGTAACAAG | |
| | | | TTCAAGGTTATTTTTGGTGGTGGCAAGCGT | |
| | | | GCCTTTTTGCCAAATACTGAACAGGACGAA | |
| | | | AAAGGATCTTATGGTAGAAGGATAGACAAC | |
| | | | CGCAATCTCATCAAAGAGTGGGAGGATGAT | |
| | | | AAGGTTTCTCGTAATGTCAGCCATCAATAT | |
| | | | GTTTGGCACCGCGAGCAGCTAATGCGTCTA | |
| | | | AAGGAGGACCTGCCTGAATACATGTTGGGA | |
| | | | CTGTTCGAGAGCAGTCATATGACCTATCAC | |
| | | | TTGAAATCAGACCCTCAGTCTGAACCCACT | |
| | | | CTCGCTGAACTAACAGAGGTGGCAATTCGG | |
| | | | TCATTAAGACGCAATGAGAAGGGATTCTTC | |
| | | | CTGTTCGTGGAGGGGGGGCGCATCGACCAC | |
| | | | GCGCACCACGACAACCTGGTGGAGCTCGCA | |
| | | | CTCGACGAGACGCTGGAGATGGACAAGGCC | |
| | | | GTGGCCACCGCCACGAAGATGCTCTCAGAG | |
| | | | GACGACTCGCTCATCGTGGTCACTGCCGAC | |
| | | | CACGCACACGTCATGACTTTCAATGGCTAC | |
| | | | TCTAACTGTGGTCATAACATCCTCGGGCCC | |
| | | | TCCAGGGATGTCGGACTAGACAATGTGCCT | |
| | | | TACATGACGCTAACGTACGCCAATGGACCC | |
| | | | GGATTCCGTCCACACGTTAACGACATTAGA | |
| | | | CCAGATGTTACCCTTGAGCCAAACTATCGC | |
| | | | ACCCTGGACTGGGAGTCGCACGTGGACGTG | |
| | | | CCGCTGGTGGACGAGACGCACGGCGGCGAC | |
| | | | GACGTGGCCGTGTTCGCGCGCGGGCCGCAC | |
| | | | CACTCCATGTTCACGGGGCTGTACGAGCAG | |
| | | | AGCCAGCTGCCGCACCTCATGGCCTACGCC | |
| | | | GCCTGCATCGGCCCCGGCCGGCACGCCTGC | |
| | | | GCCAGTGCCGCGCACTTGCCTAGCGCGCAC | |
| | | | TTCTTCGTAGCTCTGCTCGCTCTATTCACT | |
| | | | TCCATTTTACTGCGATAATATTTATTAATT | |
| | | | GAAATAGCTTTAATAAAGTTTCATACTTAA | |
| | | | TAGTCATCATTACGTTGACAAGCAGATCTG | |
| | | | TAATGTGTTGAAATAAAAGTAAAAGTTATC | |
| | | | ATTTAACATACGAAAAATAAAGTTCACATA | |
| | | | GACATTTGTAAACAATACAAGATTGATAGA | |
| | | | CGTTATTTATTTACGATTTCTGTACATACA | |
| | | | TAGGTACACATAATAAATACGAAAGGAAAG | |
| | | | AAATTAAATGTAATCGATTTTGGAGTTGAG | |
| | | | CTAATTAATAACCATGTTGTTAATATTGCG | |
| | | | AGTTTCATTGGCCACGTGCTGCATATCTAC | |
| | | | ATTGTTTGAACACATCTTCTAGGTCTTGAA | |
| | | | GCACTGCAGTTGTTGCCATTTATTGCGTAT | |
| | | | CATGTTGTTTACAATACTTAAAATATTGAA | |
| | | | CCTTTTTCATTATTAAACAATATGAACTGT | |
| | | | AAAAAAAAAAAAAAAAAAAAAAAAAAAAAA | |
| | | | AAAAAAAAAAAAAAAAAAAAAAAAAAAAAA | |
| | | | AAA | |
| 18 | Helicoverpa | Protein | MVTLFPYVVAVLCGATSARAHWLHPAAPAA | Alkaline |
| | armigera | | ASRAETSANYWAQDAQAAINARLERVESVK | phosphatase |
| | | | KARNVIMFLGDGMSVPTLAAARTLLGQRQG | |
| | | | KTGEETKLHFETFPTIGLVKTYCVDAQIAD | |
| | | | SACTATAYLCGVKNNYGAIGVDGTVRRGDC | |
| | | | QAASNTATHVESIAEWALADGRDVGIVTTT | |
| | | | RITHASPAGTFAKTANRTWENDGEVSQMGL | |
| | | | DAKDCPDIAHQLVHHHPGNKFKVIFGGGKR | |
| | | | AFLPNTEQDEKGSYGRRIDNRNLIKEWEDD | |
| | | | KVSRNVSHQYVWHREQLMRLKEDLPEYMLG | |
| | | | LFESSHMTYHLKSDPQSEPTLAELTEVAIR | |
| | | | SLRRNEKGFFLFVEGGRIDHAHHDNLVELA | |
| | | | LDETLEMDKAVATATKMLSEDDSLIVVTAD | |
| | | | HAHVMTFNGYSNCGHNILGPSRDVGLDNVP | |
| | | | YMTLTYANGPGFRPHVNDIRPDVTLEPNYR | |
| | | | TLDWESHVDVPLVDETHGGDDVAVFARGPH | |
| | | | HSMFTGLYEQSQLPHLMAYAACIGPGRHAC | |
| | | | ASAAHLPSAHFFVALLALFTSILLR | |
| 19 | Heliothis | DNA | CAGTTAGTGCGATCGCGATGATGTCGCTGT | Alkaline |
| | virescens | | ACCAGTGCCTACTGGCCGTGCTGTGCTGTG | phosphatase |
| | | | CGGCGTGCGCGCGCGCCCACTGGTTCCACC | |
| | | | CCGCAGCGACGGGGGTCGCGCGGCGGCCAC | |
| | | | CACTCGCGTCGAGACTTCTGCCAACTATTG | |
| | | | GGTGCAAGATGCGCAGGCAGCCATCGACGC | |
| | | | TCGCCTGGCGCAAGTGGAGAGCGTGAAGAA | |
| | | | AGCGCGTAACGTCATCATGTTCCTGGGCGA | |
| | | | CGGCATGTCCGTGCCCACGCTGGCCGCCGC | |
| | | | GCGCACGCTGCTAGGTCAGCGCCAAGGGAA | |
| | | | CACAGGAGAGGAGACTAAGTTGCATTTCGA | |
| | | | GACTTTTCCCACAATCGGACTAGTGAAGAC | |
| | | | ATACTGTGTGGACGCTCAGATTGCAGACTC | |
| | | | CGCATGTACTGCCACGGCGTATTTGTGTGG | |
| | | | TGTAAAGAATAATTATGGTGCTATTGGCGT | |
| | | | AGACGCCACGGTACGGCGCGGAGACTGCCA | |
| | | | GACAGCTTCAAACACTGCCACTCACGTCGA | |
| | | | GTCCATCGCGGAGTGGGCGCTCGCGGACGG | |
| | | | ACGAGATGTCGGTATCGTGACGACGACGCG | |
| | | | TATTACTCACGCGTCTCCAGCGGGCACATT | |
| | | | CGCGAAGACTGCGAACCGCACCTGGGAGAA | |
| | | | CGACGGAGAAGTATCGCAAATGGGATTGAA | |
| | | | CGCTAAGGACTGCCCTGACATCGCTCATCA | |
| | | | GTTGGTACACCACCATCCCGGTAACAAGTT | |
| | | | CAAGGTTATTTTTGGAGGTGGCAAGCGCGC | |
| | | | CTTTTTGCCAAACACTGAACAGGATGAAAA | |
| | | | AGGGTCTTACGGTAGGAGGTTAGACAACCG | |
| | | | CAACCTTATCAAAGAGTGGGAGAATGATAA | |
| | | | AGTGTCTCGTAATGTGAGTCATCAATATGT | |
| | | | TTGGAATCGCGAACAACTGATGAGCCTAAA | |
| | | | TGACGACCTGCCAGAGTACATGTTGGGCTT | |
| | | | GTTCGAGAGTAGTCATATGACATATCACAT | |
| | | | GAAATCAGATCCTCAGTCTGAACCTACTCT | |
| | | | CGCTGAACTAACAGAGTTGGCGATTCGGTC | |
| | | | ATTGCGGCGCAATGAAAAGGGATTCTTCCT | |
| | | | GTTCGTGGAGGGCGGACGCATCGACCACGC | |
| | | | GCACCACGACAACCTGGTGGAGCTCGCGCT | |
| | | | CGATGAGACGCTGGAGATGGACAAGGCCGT | |
| | | | GGCGACCGCCACGCAGCTGCTGTCGGAGGA | |
| | | | CGACTCGCTCATTGTGGTCACCGCCGACCA | |
| | | | CGCACACGTCATGACTTTCAATGGCTACTC | |
| | | | TAACCGTGGCCGTGACATCTTGGGGCCCTC | |
| | | | CAGGGATGTTGATCTAGACAACGTACCTTA | |
| | | | CATGACGCTAACGTATGCCAATGGACCTGG | |
| | | | ATTTCGTTCGCACGTGAACGACATTCGACC | |
| | | | AGATGTTACAGCTGAGTCAAACTACCGCTC | |
| | | | TCTGGACTGGGAGTCGCACGTGGACGTGCC | |
| | | | GCTGGAGGACGAGACGCACGGCGGTGACGA | |
| | | | CGTGGCCGTGTTCGCGCGCGGGCCGCGCCA | |
| | | | CTCCATGTTCACGGGGCTGTACGAGCAGAG | |
| | | | CCAGCTGCCGCACCTCATGGCCTACGCCGC | |
| | | | CTGCATCGGCCCCGGCCGACACGCCTGCGT | |
| | | | CAGCGCCGCGCACTTGCCCACCGCGCACTT | |
| | | | CTTTATTGCTCTGTTTGCTCTATTCACCCC | |
| | | | GATTTTACTAAAATAATAATTATTTAGAAT | |
| | | | TTACATCATAAAAAAAAAAAAAAAAAAAA | |
| 20 | Heliothis | Protein | MMSLYQCLLAVLCCAACARAHWFHPAATAG | Alkaline |
| | virescens | | RAAATTRVETSANYWVQDAQAAIDARLAQV | phosphatase |
| | | | ESVKKARNVIMFLGDGMSVPTLAAARTLLG | |
| | | | QRQGNTGEETKLHFETFPTIGLVKTYCVDA | |
| | | | QIADSACTATAYLCGVKNNYGAIGVDATVR | |
| | | | RGDCQTASNTATHVESIAEWALADGRDVGI | |
| | | | VTTTRITHASPAGTFAKTANRTWENDGEVS | |
| | | | QMGLNAKDCPDIAHQLVHHHPGNKFKVIFG | |
| | | | GGKRAFLPNTEQDEKGSYGRRLDNRNLIKE | |
| | | | WENDKVSRNVSHQYVWNREQLMSLNDDLPE | |
| | | | YMLGLFESSHMTYHMKSDPQSEPTLAELTE | |
| | | | LAIRSLRRNEKGFFLFVEGGRIDHAHHDNL | |
| | | | VELALDETLEMDKAVATATQLLSEDDSLIV | |
| | | | VTADHAHVMTFNGYSNRGRDILGPSRDVDL | |
| | | | DNVPYMTLTYANGPGFRSHVNDIRPDVTAE | |
| | | | SNYRSLDWESHVDVPLEDETHGGDDVAVFA | |
| | | | RGPHHSMFTGLYEQSQLPHLMAYAACIGPG | |
| | | | RHACVSAAHLPTAHFFIALFALFTPILLK | |
| 21 | Spodoptera | DNA | ATGAGGTCGCTACTGACTTACCTAGTGGCC | Alkaline |
| | frugiperda | | GCCGTGGTGGTGGCGGCGTGTGTCCGCGGG | phosphatase |
| | | | GACCGGTACCACCCCGCGGACCCCGGCAGC | |
| | | | AGAGCTGACACCGTTGCGAACCGTGCCGAG | |
| | | | ACCTCAGCCAACTACTGGGCCCAAGAAGCG | |
| | | | CAGGCTGCAATCAATGCCCGGCTGGCGCAC | |
| | | | AAGGAGAGCGTGAAGAAGGCGCGCAACGTG | |
| | | | GTCATGTTCCTGGGCGACGGCATGTCCGTG | |
| | | | CCCACGCTCGCCGCCGCGCGGACGCTGCTC | |
| | | | GGCCAGCGCCGCGGGCACACCGGCGAGGAG | |
| | | | GATAAACTGCATTTTGAAACATTCCCCACC | |
| | | | GTTGGATTGACTAAGACGTATTGCGTGAAC | |
| | | | GCTCAGATCCCAGACTCCGCGTGCACTGCT | |
| | | | ACTGCGTACTTATGTGGTGTCAAAACAACT | |
| | | | TACGGAGCTATTGGAGTGAATGCGGAGGTG | |
| | | | CCACGGAAAGGCTGCGAGGCGTCCACCGAC | |
| | | | ACCAGCCGACACGTGGAGTCCATCGCGGAG | |
| | | | TGGGCGCTGGCCGACGGCCGCGACGCTGGT | |
| | | | ATCGTGACGACGACGCGCATCACCCACGCG | |
| | | | TCGCCGGCCGGCGTGTACGCCAAGGTGGCG | |
| | | | GACCGCAACTGGGAGCACAACCAGGCGGTG | |
| | | | GAGAACGATGGCTTCGACACGGACAAGTGC | |
| | | | CCGGATATCGCACTGCAGCTCGTGCATAAG | |
| | | | CACCCCGGGAATAAACTCAAGGTTATTTTA | |
| | | | GGCGGAGGAAGACTAAACTTTTTGCCAAAT | |
| | | | GATGTGAAAGACGAAGAAGGGGTATATGGA | |
| | | | AACCGAACAGACAGCCGCAACCTCATCGAA | |
| | | | GAATGGGCACAAGACAAGGAAGATCGTAAA | |
| | | | GTTACTCATAAATATGTTTGGAATCGTGAG | |
| | | | CAGCTGATGAGTCTTAAAGATGATCTTCCT | |
| | | | GAGTACCTTTTAGGACTTTTCGAAAGTAAT | |
| | | | CATCTTCAGTACAACATGCAGGCAGATCCT | |
| | | | AATACTGAGCCCACGCTGACTGAGCTAACT | |
| | | | GAGATAGCAATCAAGTCGCTAAGTARAAAC | |
| | | | GAGAAAGGTTTTTTCCTGTTCGTGGAAGGC | |
| | | | GGTCGTATCGACCACGCGCACCATCGCAAC | |
| | | | TGGGTAGAGCTAGCGCTGGACGAGACCCTG | |
| | | | GAGATGGACGAGGCCGTCGCGCGCGCCGCC | |
| | | | GAGCTGCTCCCCGAGGACGACTCGCCCATT | |
| | | | GTGGTCACAGONNACCACTCCCACGTCATG | |
| | | | GCTTACAATGGATACTCGGCCCGTGGACAT | |
| | | | GACATCCTCGGCCCTTCCAGAGACTTGGAC | |
| | | | CTGGACGGAGTGCCTTACATGACGCTGTCG | |
| | | | TACACCAACGGGCCCGGCTTCCGTTCGCAT | |
| | | | ATGAACGGTATACGCCCCGATGTCACCGCT | |
| | | | GAAGACGGTTTCGGAGAAGACGAATGGTTG | |
| | | | GCTCATGTAGATGTTCCGTTGATAGACGAG | |
| | | | ACGCACGGCGGGGACGACGTGGCGGTGTTC | |
| | | | GCGCGCGGGCCGCACCACTCCATGTTCACG | |
| | | | GGGCTGTACGAGCAGAGCCAGCTGCCGCAC | |
| | | | CTCATGGCGTACGCCGCCTGCATCGGCCCC | |
| | | | GGCAGACACGCCTGCAGCGGCGCCGCGCAT | |
| | | | GCGCTGGCCCAGCCTGTGCTGCTGCTCTCT | |
| | | | CTCCTTGTACTGCTCACTTCACTATTCCAA | |
| | | | CAATGA | |
| 22 | Spodoptera | Protein | MRSLLTYLVAAVVVAACVRGDRYHPADPGS | Alkaline |
| | frugiperda | | RADTVANRAETSANYWAQEAQAAINARLAH | phosphatase |
| | | | KESVKKARNVVMFLGDGMSVPTLAAARTLL | |
| | | | GQRRGHTGEEDKLHFETFPTVGLTKTYCVN | |
| | | | AQIPDSACTATAYLCGVKTTYGAIGVNAEV | |
| | | | PRKGCEASTDTSRHVESIAEWALADGRDAG | |
| | | | IVTTTRITHASPAGVYAKVADRNWEHNQAV | |
| | | | ENDGFDTDKCPDIALQLVHKHPGNKLKVIL | |
| | | | GGGRLNFLPNDVKDEEGVYGNRTDSRNLIE | |
| | | | EWAQDKEDRKVTHKYVWNREQLMSLKDDLP | |
| | | | EYLLGLFESNHLQYNMQADPNTEPTLTELT | |
| | | | EIAIKSLSXNEKGFFLFVEGGRIDHAHHRN | |
| | | | WVELALDETLEMDEAVARAAELLPEDDSPI | |
| | | | VVTAXHSHVMAYNGYSARGHDILGPSRDLD | |
| | | | LDGVPYMTLSYTNGPGFRSHMNGIRPDVTA | |
| | | | EDGFGEDEWLAHVDVPLIDETHGGDDVAVF | |
| | | | ARGPHHSMFTGLYEQSQLPHLMAYAACIGP | |
| | | | GRHACSGAAHALAQPVLLLSLLVLLTSLFQ | |
| | | | Q | |
| 23 | Heliothis | DNA | ATGGGCGTAGAAAATAAGAATAATGTACAA | ABCC2 |
| | virescens | | AATGCGGAAGGCGCGGCCCTCAAGACTTAC | |
| | | | AAGAAACCGAACATTTTATCTCGTATATTT | |
| | | | CTTTGGTGGATGTGTCCGGTGCTCATAACT | |
| | | | GGTAACAGAAGAAACGTAGAAGAATCAGAT | |
| | | | CTAATACCGCCCAGTAATTTATATAATTCA | |
| | | | GAAAGACAAGGAGAATATCTAGAAAGATAC | |
| | | | TGGTTACAAGAAATAGAAAATGCAACAAAT | |
| | | | GAAAATCGGGAGCCATCGCTATGGAAGGCG | |
| | | | TTGCAAAGGGCCTACTGGGTATCCTATATG | |
| | | | CCAGGAGCCATTTATGTCTTAATTCAATCA | |
| | | | GCAGCCAGGACGTATCAGCCGTTGTTATTT | |
| | | | GCTCAGCTACTGACGTACTGGTCGGTCGAT | |
| | | | AGTGAAATGACTCAGCAAGACGCTGGGCTG | |
| | | | TATGCGCTAGCCATGCTGGGACTGAACTTC | |
| | | | GTCTCCATGATGTGTCAGCACCACAACAAC | |
| | | | TTGTTTGTGATGCGGTTCAGTTTGAAAGTC | |
| | | | AAGGTTGCTTGTTCTTCACTCTTGTATAGG | |
| | | | AAGTTGCTCCGCATGACTCAAGTGTCGGTA | |
| | | | AGTGAAGTCGCAGGTGGAAAGTTGGTAAAC | |
| | | | TTGCTGTCCAACGACATCACGAGGTTCGAC | |
| | | | TACGCATTCATGTTCCTGCACTACTTGTGG | |
| | | | ATAGTGCCTATCCAAGTGGCCGTAGTCTTG | |
| | | | TACTTCTTGTGGGATGCTGCTGGGTTTGCG | |
| | | | CCTTTTGTTGGTCTCTTTGGAGTTGTCCTA | |
| | | | TTGATTTTACCACTTCAAGCCGGTTTGACG | |
| | | | AGACTCACATCTATTGTAAGGCGTGAGACA | |
| | | | GCCAAGAGGACGGATAGGCGAATTAAACTT | |
| | | | ATGAGTGAAATTATCAATGGTATTCAGGTC | |
| | | | ATTAAAATGTACGCTTGGGAGAAACCCTTC | |
| | | | CAGCTAGTGGTGAAGGCGGCTCGTGCCTTT | |
| | | | GAAATGAGTGCTCTCAGGAAGTCCATCTTC | |
| | | | ATTAGGAGTACCTTCCTCGGGTTCATGTTG | |
| | | | TTCACTGAGCGGAGTATCATGTTTGTCACA | |
| | | | GTATTGACACTCGCACTCACAGGCACTATG | |
| | | | ATTACTGCTACCACGATATATCCCATTCAG | |
| | | | CAGTACTTCAGTATTATTCAATTTAATGTA | |
| | | | ACGCTAGTCATTCCTATGGCAATCGCAAGT | |
| | | | TTATCCGAGATGATGGTGTCTATAGAACGT | |
| | | | ATCCAAGGATTCCTCAGTTTGGATGAGCGG | |
| | | | TCCGACATGCAAGTGACTCCTAAAATGATG | |
| | | | GCTCCAATAACAGCACTTTGTTCAAAAGCA | |
| | | | AGAAGGCACCACTTGAAATAAGCATCGTGC | |
| | | | CAAAGAAATACTCGCCTAGCGAAGTTACGG | |
| | | | TTGCAAGAGAAGTGCAGGATGATCCCAGCC | |
| | | | AGGTGGACTATCCTATAAGACTCAACAAAA | |
| | | | TAAACGCATCGTGGACCGGCAATGATACTC | |
| | | | CTTCAGAGATGACACTTAAAAATATATCCT | |
| | | | TACGTATACGTAAAGGCAAATTGTGTGCTA | |
| | | | TCATTGGTCCTGTGGGTTCCGGAAAGACGT | |
| | | | CTCTGCTTCAGCTACTCTTAAAAGAATTAC | |
| | | | CGATGACTAGTGGCACACTGGACGTGTCAG | |
| | | | GAAGATTGTCTTACGCTTGTCAGGAGTCGT | |
| | | | GGCTGTTCCCCGGCACAGTGCGAGAAAACA | |
| | | | TTTTGTTTGGCCTAGATTATGAAGCCACAA | |
| | | | AATACAAAGAGGTTTGCAAGGTGTGTTCGT | |
| | | | TACTGCCAGACTTCAAACAGTTCCCGTATG | |
| | | | GTGACTTGTCTTTAGTGGGGGAGCGAGGAG | |
| | | | TATCCCTGTCTGGAGGTCAAAGAGCCAGAA | |
| | | | TCAATTTAGCCAGAGCAATTTATCGTGAGG | |
| | | | CCGATATTTACTTGCTGGATGATCCCCTAT | |
| | | | CCGCAGTGGATGCAAATGTAGGCAGACAAC | |
| | | | TGTTTGATGGCTGCATCAAAGGCTACCTTT | |
| | | | CTGGACGAACTTGCGTCTTGGTCACCCATC | |
| | | | AGATCCATTACCTCAAAGCTGCTGATTTTA | |
| | | | TTGTAGTCCTTAATGAGGGTTCCATCGAGA | |
| | | | ACATGGGCACGTATGACGAGCTGGTCAAGA | |
| | | | CAGGAACCGAGTTCTCGATGCTGCTATCCA | |
| | | | ACCAAGAAAGTGAAGCAACTGAAAACGAAA | |
| | | | TGAAAGAGCGACCATCATTGCTGCGAGGAA | |
| | | | TATCAAAAATCTCAATTAAGAGCGACGACC | |
| | | | ACGATGCGGATCAGAAGGCGCAAGTACAGG | |
| | | | AGGCAGAGGAGAGAGCAACAGGCAGCTTGA | |
| | | | AGTGGGAGGTGGTGCTGAAGTACCTGAGCT | |
| | | | CCGTCGAATCGTGGTGTCTGGTGTTCATGG | |
| | | | CTTTCCTCGCGCTGCTGATCACGCAAGGCG | |
| | | | CTGCCACTACGTCTGACTATTGGCTGAGTT | |
| | | | TCTGGACTAATCAAGTGGATTCTTATGAAC | |
| | | | AATCATTGCCTGATGGCGCCGAACCAGATA | |
| | | | CTGACATGAACGCACAAATTGGTTTACTTA | |
| | | | CAACTGCCCAGTACCTATACGTGTACGGCG | |
| | | | GAGTTATATTGGCTGTAATAATCATGACAC | |
| | | | TCGTCAGGATCACAGGTTTCGTAGCGATGA | |
| | | | CAATGCGAGCCTCTCAAAATCTCCACAACA | |
| | | | CTATTTACGAAAAATTGATTGTGACAGTAA | |
| | | | TGAGATTCTTTGATACCAATCCTTCTGGTC | |
| | | | GTGTCCTGAACAGGTTCTCAAAAGACATGG | |
| | | | GTGCCATGGATGAGCTTCTACCTCGCAGTC | |
| | | | TTTTAGAAACAGTTCAGATGTATCTGTCGC | |
| | | | TGACCAGCATCTTGGTGCTAAACGCCACAG | |
| | | | CATTGCCCTGGACGCTCATACCTACCTCCG | |
| | | | TATTGATAGTCATCTTCGTGTTCATGTTGA | |
| | | | GATGGTACCTGAATACAGCTCAGGCTGTCA | |
| | | | AACGATTGGAAGGAACAACCAAGAGTCCTG | |
| | | | TATTTGGAATGATTAATTCCACTATTTCTG | |
| | | | GACTTTCGACTATCAGGAGTTCGGGTTCCC | |
| | | | AGTTTAGACAGATGAGATTATTTGACGAAG | |
| | | | CGCAGAATCTCCACACAAGTGCTTTCCACA | |
| | | | CATTCTTTGGCGGTTCTACGGCATTTGGAT | |
| | | | TGTATCTCGATACTTTGTGTTTGATCTACC | |
| | | | TCGGAGTTGTCATGTCAATTTTCATTTTGG | |
| | | | GCGACTTTGGTGATTTGATCGCAGTAGGAA | |
| | | | GCGTCGGTCTGGCCGTCAGTCAGTCCATGG | |
| | | | TGCTCACCATGATGTTGCAGATGACAGCTA | |
| | | | GGTTTACAGCTGACTTCTTGGGACAAATGA | |
| | | | CGGCAGTAGAGAGGGTGCTGGAGTACACCC | |
| | | | AGCTACCCATGGAGACTAATATGGAGCAAG | |
| | | | GACCAACTAACCCGCCAAAAGAATGGCCTA | |
| | | | ATGCTGGCAGAGTGACGTTCTCAAATGTGT | |
| | | | ACCTGAATTATTCTGTGGAAGACCCACCAG | |
| | | | TGCTAAAGGACTTGAACTTTGAAATTCAAA | |
| | | | GCGGTTGGAAGGTTGGAGTTGTAGGCCGAA | |
| | | | CGGGAGCCGGCAAATCATCGCTCATTGCGG | |
| | | | CTCTGTTCCGGCTTACCGACATAACTGGCA | |
| | | | GCATCAAAATTGACGGCGTGGACACAGAAG | |
| | | | GATTAGCCAAAAAGCTTTTGAGATCGAAAA | |
| | | | TATCAATAATTCCACAAGAGCCGGTCCTCT | |
| | | | TCTCCGCTACTCTGCGTTATAATTTGGATC | |
| | | | CGTTCGACGATTACAGTGACGAAGATATTT | |
| | | | GGAGGGCTCTGGAACAGGTGGAACTAAAAG | |
| | | | AAGGAATACCGGCACTTGATTATAAAGTGG | |
| | | | CTGAAGGTGGTACTAACTTCTCCATGGGAC | |
| | | | AGCGTCAGTTGGTATGCTTGGCTCGTGCTA | |
| | | | TTTTACGCTCTAACAAAATACTCATCATGG | |
| | | | ACGAAGCCACAGCTAATGTCGATCCTCAGA | |
| | | | CGGACGCTTTGATTCAAAAGACGATCCGTC | |
| | | | GTCAATTCGCGTCGTGCACGGTGCTGACCA | |
| | | | TCGCGCATCGACTGAACACCATCATGGACT | |
| | | | CCGACCGAGTGCTGGTCATGGACCAGGGCG | |
| | | | AGGTCGCCGAGTTCGATCACCCACACATCT | |
| | | | TGCTCAGCAACCCCAACAGCAAGTTCTTCT | |
| | | | CTATGGTCAGAGAGACCGGAGAAAGCATGA | |
| | | | CGAAGACCTTAATGGAGGTCGCGAAGACTA | |
| | | | AATACGATAGTGATAATAAGGAGGCTTAG | |
| 24 | Heliothis | Protein | MGVENKNNVQNAEGAALKTYKKPNILSRIF | ABCC2* |
| | virescens | | LWWMCPVLITGNRRNVEESDLIPPSNLYNS | |
| | | | ERQGEYLERYWLQEIENATNENREPSLWKA | |
| | | | LQRAYWVSYMPGAIYVLIQSAARTYQPLLF | |
| | | | AQLLTYWSVDSEMTQQDAGLYALAMLGLNF | |
| | | | VSMMCQHHNNLFVMRFSLKVKVACSSLLYR | |
| | | | KLLRMTQVSVSEVAGGKLVNLLSNDITRFD | |
| | | | YAFMFLHYLWIVPIQVAVVLYFLWDAAGFA | |
| | | | PFVGLFGVVLLILPLQAGLTRLTSIVRRET | |
| | | | AKRTDRRIKLMSEIINGIQVIKMYAWEKPF | |
| | | | QLVVKAARAFEMSALRKSIFIRSTFLGFML | |
| | | | FTERSIMFVTVLTLALTGTMITATTIYPIQ | |
| | | | QYFSIIQFNVTLVIPMAIASLSEMMVSIER | |
| | | | IQGFLSLDERSDMQVTPKMNGSNNSTLFKS | |
| | | | KKAPLEISIVPKKYSPSEVTVAREVQDDPS | |
| | | | QVDYPIRLNKINASWTGNDTPSEMTLKNIS | |
| | | | LRIRKGKLCAIIGPVGSGKTSLLQLLLKEL | |
| | | | PMTSGTLDVSGRLSYACQESWLFPGTVREN | |
| | | | ILFGLDYEATKYKEVCKVCSLLPDFKQFPY | |
| | | | GDLSLVGERGVSLSGGQRARINLARAIYRE | |
| | | | ADIYLLDDPLSAVDANVGRQLFDGCIKGYL | |
| | | | SGRTCVLVTHQIHYLKAADFIVVLNEGSIE | |
| | | | NMGTYDELVKTGTEFSMLLSNQESEATENE | |
| | | | MKERPSLLRGISKISIKSDDHDADQKAQVQ | |
| | | | EAEERATGSLKWEVVLKYLSSVESWCLVFM | |
| | | | AFLALLITQGAATTSDYWLSFWTNQVDSYE | |
| | | | QSLPDGAEPDTDMNAQIGLLTTAQYLYVYG | |
| | | | GVILAVIIMTLVRITGFVAMTMRASQNLHN | |
| | | | TIYEKLIVTVMRFFDTNPSGRVLNRFSKDM | |
| | | | GAMDELLPRSLLETVQMYLSLTSILVLNAT | |
| | | | ALPWTLIPTSVLIVIFVFMLRWYLNTAQAV | |
| | | | KRLEGTTKSPVFGMINSTISGLSTIRSSGS | |
| | | | QFRQMRLFDEAQNLHTSAFHTFFGGSTAFG | |
| | | | LYLDTLCLIYLGVVMSIFILGDFGDLIAVG | |
| | | | SVGLAVSQSMVLTMMLQMTARFTADFLGQM | |
| | | | TAVERVLEYTQLPMETNMEQGPTNPPKEWP | |
| | | | NAGRVTFSNVYLNYSVEDPPVLKDLNFEIQ | |
| | | | SGWKVGVVGRTGAGKSSLIAALFRLTDITG | |
| | | | SIKIDGVDTEGLAKKLLRSKISIIPQEPVL | |
| | | | FSATLRYNLDPFDDYSDEDIWRALEQVELK | |
| | | | EGIPALDYKVAEGGTNFSMGQRQLVCLARA | |
| | | | ILRSNKILIMDEATANVDPQTDALIQKTIR | |
| | | | RQFASCTVLTIAHRLNTIMDSDRVLVMDQG | |
| | | | EVAEFDHPHILLSNPNSKFFSMVRETGESM | |
| | | | TKTLMEVAKTKYDSDNKEA | |
| 25 | Helicoverpa | DNA | ATGGGCGTAGAAAATAAGAATAATGTACAA | ABCC2* |
| | armigera | | AATGCGGAAGGTCCGGCCCGCAAGACTTAC | |
| | | | AAAAGACCGAACATTTTATCCCGTATATTT | |
| | | | CTCTGGTGGATGTGTCCTGTGCTCATAACT | |
| | | | GGTAACAAAAGAAATGTAGAAGAATCAGAT | |
| | | | CTTATACCGCCCAGTAATTTATATAATTCA | |
| | | | GAAAGACAAGGAGAATATCTTGAAAGATAC | |
| | | | TGGTTGGCAGAGATAGAAAATGCAACAATT | |
| | | | GAAAATCGAGAGCCATCACTTTGGAAGGCA | |
| | | | TTACGAAAGGCCTACTGGGTCTCCTATATG | |
| | | | CCAGGAGCTATTTTTATCATCATTCAATCT | |
| | | | GCAGCCAGGACGTATCAGCCGCTGTTGTTT | |
| | | | TCTCAGCTTTTGTCGTACTGGTCAGTGGAC | |
| | | | AGTGAAATGACTCAGCAAGACGCTGGCCTG | |
| | | | TATGCTCTCGCCATGCTGGGACTGAACTTC | |
| | | | GTCTCTATGATGTGTCAGCACCACAACACA | |
| | | | TTGTTTGTGATGCGGTTCAGTTTAAAAGTC | |
| | | | AAGGTTGCCTGTTCTTCGCTCTTGTATAGG | |
| | | | AAGTTGCTCCGCATGACCCAAGTGTCAGTA | |
| | | | GGTGAGGTGGCTGGTGGAAAGTTGGTGAAC | |
| | | | TTGCTGTCCAACGATATCACGAGGTTCGAC | |
| | | | TACGCCTTCATGTTCCTTCACTACTTGTGG | |
| | | | ATCGTGCCTATCCAAGTGGCTGTTGTCTTA | |
| | | | TACTTCTTGTGGGAAGCTGCTGGCTTTGCG | |
| | | | CCCTTCGTCGGTCTGTTTGGAGTCGTTATA | |
| | | | CTGATTTTACCACTGCAAGCTGGCTTGACG | |
| | | | AAACTCACAACTGTTGTAAGACGTGAGACG | |
| | | | GCTAAGAGAACGGACAGGCGAATTAAACTA | |
| | | | ATGAGTGAAATTATTGGTGGTATTCAGGTC | |
| | | | ATTAAAATGTACGCTTGGGAGAAACCCTTC | |
| | | | CAGCTAGTIGTGAAAGCAGCCCGTGCCTTC | |
| | | | GAAATGGGTGCCCTCAGGAAGTCCATCTTC | |
| | | | ATCAGGAGTACTTTCCTAGGGTTCATGTTG | |
| | | | TTCACTGAAAGAAGCATCATGTTTGTCACA | |
| | | | GTGTTGACACTCGCTCTCACAGGCACTATG | |
| | | | ATTACTGCTACCACGATATATCCTATTCAA | |
| | | | CAGTACTTCAGTATTATTCAGTTTAACGTA | |
| | | | ACACTGATCATTCCTATGGCAATCGCAAGT | |
| | | | TATTCCGAGATGATGGTGTCCATAGAACGT | |
| | | | ATCCAGGGATTCCTTAGTTTGGACGAGCGG | |
| | | | TCAGACATGCAAGTGACTCCAAAAATGAAT | |
| | | | GGATCTAACAATAACACTTTGTTTAAATCC | |
| | | | AAGAAGTCACCACTTGAAGTAGGCATCGTG | |
| | | | CCTAAAAAATACTCACCAAGCGAAGTTATG | |
| | | | GCTGCAAAGGAGATGCAGGATGATCCTACC | |
| | | | CAGATGGACTATCCTATCAGACTCAACAAA | |
| | | | GTAAGCGCATCCTGGACCGGCAGCAATAGT | |
| | | | TCTTCAGAAATGACACTTAAGAATATATCG | |
| | | | TTGCGTATACGTAAAGGAAAATTGTGTGCT | |
| | | | ATCATTGGTCCTGTGGGGTCCGGAAAGACG | |
| | | | TCTCTACTGCAACTGCTGTTAAAAGAATTA | |
| | | | CCATTGAACAGTGGTACACTTGACGTGAGT | |
| | | | GGGAAGATGTCCTACGCTTGTCAAGAGTCC | |
| | | | TGGCTGTTCCCGGGCACAGTGCGAGAAAAC | |
| | | | ATTTTGTTTGGCCTAACTTACGAACCCACA | |
| | | | AAATACAAGGAGGTTTGCAAGGTGTGTTCG | |
| | | | TTACTGCCGGATTTCAAGCAGTTCCCGTAT | |
| | | | GGTGACCTGTCCTTGGTGGGAGAGAGAGGA | |
| | | | GTATCCTTGTCAGGTGGTCAAAGAGCCAGG | |
| | | | ATCAATTTGGCCAGAGCAATTTATCGTGAG | |
| | | | GCCGACATTTACTTGCTGGATGATCCTCTA | |
| | | | TCTGCAGTGGACGCTAATGTAGGCAGACAA | |
| | | | CTGTTTGACGGCTGCATCAAAGGCTACCTC | |
| | | | ACTGGAAGAACGTGCGTCTTGGTCACCCAT | |
| | | | CAGATCCATTACCTCAAAGCTGCTGATTTT | |
| | | | ATTGTAGTCCTTAATGAGGGCTCCGTCGAG | |
| | | | AATATGGGCACGTATGATGAGCTGGTGAAG | |
| | | | ACAGGAACTGAGTTCTCAATGCTGCTATCC | |
| | | | AACCAAGAAAATGACGCAACTGAAAACGAA | |
| | | | AAGAAAGATCGACCAGCAATGATGCGAGGA | |
| | | | ATATCAAAAATCTCAGTTAAGAGCGACACC | |
| | | | GAAATGGAACAGAAGGCTCAAATACAGGAG | |
| | | | GCAGAGGAGAGAGCGACAGGTAGCTTGAAG | |
| | | | TTCGAGGTGGTGCTCAAGTATCTGAGCTCA | |
| | | | GTCCAGTCCTGGTGTCTGGTGTTCACGGCG | |
| | | | TTCCTGGTGCTGCTGATCACGCAAGGCGCT | |
| | | | GCCACCACGGCCGACTATTGGTTGAGTTTC | |
| | | | TGGACTAATCAAGTGGATTCTTATGAACAA | |
| | | | TCGTTGCCTGATGGCGTTGATCCAGATACT | |
| | | | GACATGAATGCACAAATTGGTTTACTTACA | |
| | | | ACTGCCCAGTACCTATACGTTTTCGGTGGA | |
| | | | GTTATATTGGCTTTGATAGTCATGACCCTC | |
| | | | GTCAGAATCACAGCTTTCGTAGCGATGACA | |
| | | | ATGCGAGCTTCTCAAAATCTTCACAACACT | |
| | | | ATTTACGAAAAATTGATTGTGACTGTAATG | |
| | | | AGATTTTTCGATACTAATCCATCTGGTCGT | |
| | | | GTCCTGAACAGGTTCTCAAAAGACATGGGT | |
| | | | GCTATGGATGAGCTTCTACCTCGCAGTCTT | |
| | | | TTAGAAACAATTCAGATGTATCTGTCTCTG | |
| | | | ACCAGCGTGTTGGTGCTAAACGCCACAGCA | |
| | | | TTGCCCTGGACGCTCATTCCTACCTCCGTG | |
| | | | TTGATTGTCATCTTCGTGCTCATGTTGAGA | |
| | | | TGGTACCTGAATACTGCTCAGGCTGTGAAA | |
| | | | CGTTTGGAAGGCACAACCAAGAGTCCTGTA | |
| | | | TTTGGAATGATTAACTCCACTATTTCTGGA | |
| | | | CTCTCCACTATCAGAAGTTCTGGTTCCCAG | |
| | | | GATAGACAGATGAAATTGTTTGACGAAGCG | |
| | | | CAGAATCTCCACACAAGTGCTTTCCACACA | |
| | | | TTCTTCGGCGGTTCTACGGCATTTGCACTG | |
| | | | TATCTCGATACTTTGTGTTTGTTCTACCTC | |
| | | | GGAGTTGTCATGTCAATCTTCATTTTGGGC | |
| | | | GACTTTGGTGATTTGATCCCGGTGGGAAGC | |
| | | | GTCGGTCTGGCCGTCAGTCAGTCCATGGTG | |
| | | | CTGACCATGATGTTGCAGATGGCAGCCAGG | |
| | | | TTTACAGCTGACTTCTTGGGACAAATGACG | |
| | | | GCAGTGGAGAGAGTGCTGGAGTACACCAAA | |
| | | | CTACCCACGGAAACCAATATGGAACAAGGA | |
| | | | CCAACTAACCCACCAAAAGAATGGCCCAGT | |
| | | | GCTGGTAGAGTGACGTTCTCAAATGTGTAC | |
| | | | CTGAATTATTCCATGGAAGACCCACCGGTG | |
| | | | CTGAAGGACTTAAGCTTTGAAATTCAAAGC | |
| | | | GGTTGGAAGGTTGGAGTTGTAGGCAGAACT | |
| | | | GGAGCCGGCAAGTCATCACTCATCGCGGCT | |
| | | | TTGTTCCGGCTTAGCGACATAAGCGGCAGC | |
| | | | ATCAAAATTGACGGCGTGGACACCGAAGGA | |
| | | | TTAGCCAAAAAGACTTTGAGATCGAAAATA | |
| | | | TCAATTATTCCACAAGAACCGGTGCTGTTC | |
| | | | TCGGCTACTCTGCGATACAATTTGGATCCA | |
| | | | TTCGACGATTACAGCGACGACGATATTTGG | |
| | | | AGGGCGTTGGAACAGGTGGAATTAAAAGAA | |
| | | | GGAATACCGGCTTTAGACTTTAAGGTCGCT | |
| | | | GAAGGTGGTACTAACTTCTCTATGGGACAA | |
| | | | CGTCAACTGGTGTGCTTGGCGCGTGCCATT | |
| | | | CTTCGGTCTAATAAAATACTCATCATGGAC | |
| | | | GAAGCTACCGCTAATGTCGATCCTCAGACG | |
| | | | GACGCTTTGATCCAAAAGACGATCCGTCGC | |
| | | | CAGTTCGCGTCGTGCACGGTGCTCACCATC | |
| | | | GCGCATCGACTGAACACCATCATGGACTCC | |
| | | | GACCGAGTGCTGGTCATGGACCAGGGCGAG | |
| | | | GTGGCCGAGTTCGACCACCCCCACATCTTG | |
| | | | CTCAGCAACCCCAATAGCAAGTTCTTCTCT | |
| | | | ATGGTCCGGGAGACAGGAGAAAGCATGACA | |
| | | | AGGACCTTAATGGAGGTCGCTAAGGCCAAA | |
| | | | TATGATAGTGATAATAAGGAGGCTTAA | |
| 26 | Helicoverpa | Protein | MGVENKNNVQNAEGPARKTYKRPNILSRIF | ABCC2* |
| | armigera | | LWWMCPVLITGNKRNVEESDLIPPSNLYNS | |
| | | | ERQGEYLERYWLAEIENATIENREPSLWKA | |
| | | | LRKAYWVSYMPGAIFIIIQSAARTYQPLLF | |
| | | | SQLLSYWSVDSEMTQQDAGLYALAMLGLNF | |
| | | | VSMMCQHHNTLFVMRFSLKVKVACSSLLYR | |
| | | | KLLRMTQVSVGEVAGGKLVNLLSNDITRFD | |
| | | | YAFMFLHYLWIVPIQVAVVLYFLWEAAGFA | |
| | | | PFVGLFGVVILILPLQAGLTKLTTVVRRET | |
| | | | AKRTDRRIKLMSEIIGGIQVIKMYAWEKPF | |
| | | | QLVVKAARAFEMGALRKSIFIRSTFLGFML | |
| | | | FTERSIMFVTVLTLALTGTMITATTIYPIQ | |
| | | | QYFSIIQFNVTLIIPMAIASYSEMMVSIER | |
| | | | IQGFLSLDERSDMQVTPKMNGSNNNTLFKS | |
| | | | KKSPLEVGIVPKKYSPSEVMAAKEMQDDPT | |
| | | | QMDYPIRLNKVSASWTGSNSSSEMTLKNIS | |
| | | | LRIRKGKLCAIIGPVGSGKTSLLQLLLKEL | |
| | | | PLNSGTLDVSGKMSYACQESWLFPGTVREN | |
| | | | ILFGLTYEPTKYKEVCKVCSLLPDFKQFPY | |
| | | | GDLSLVGERGVSLSGGQRARINLARAIYRE | |
| | | | ADIYLLDDPLSAVDANVGRQLFDGCIKGYL | |
| | | | TGRTCVLVTHQIHYLKAADFIVVLNEGSVE | |
| | | | NMGTYDELVKTGTEFSMLLSNQENDATENE | |
| | | | KKDRPAMMRGISKISVKSDTEMEQKAQIQE | |
| | | | AEERATGSLKFEVVLKYLSSVQSWCLVFTA | |
| | | | FLVLLITQGAATTADYWLSFWTNQVDSYEQ | |
| | | | SLPDGVDPDTDMNAQIGLLTTAQYLYVFGG | |
| | | | VILALIVMTLVRITAFVAMTMRASQNLHNT | |
| | | | IYEKLIVTVMRFFDTNPSGRVLNRFSKDMG | |
| | | | AMDELLPRSLLETIQMYLSLTSVLVLNATA | |
| | | | LPWTLIPTSVLIVIFVLMLRWYLNTAQAVK | |
| | | | RLEGTTKSPVFGMINSTISGLSTIRSSGSQ | |
| | | | DRQMKLFDEAQNLHTSAFHTFFGGSTAFAL | |
| | | | YLDTLCLFYLGVVMSIFILGDFGDLIPVGS | |
| | | | VGLAVSQSMVLTMMLQMAARFTADFLGQMT | |
| | | | AVERVLEYTKLPTETNMEQGPTNPPKEWPS | |
| | | | AGRVTFSNVYLNYSMEDPPVLKDLSFEIQS | |
| | | | GWKVGVVGRTGAGKSSLIAALFRLSDISGS | |
| | | | IKIDGVDTEGLAKKTLRSKISIIPQEPVLF | |
| | | | SATLRYNLDPFDDYSDDDIWRALEQVELKE | |
| | | | GIPALDFKVAEGGTNFSMGQRQLVCLARAI | |
| | | | LRSNKILIMDEATANVDPQTDALIQKTIRR | |
| | | | QFASCTVLTIAHRLNTIMDSDRVLVMDQGE | |
| | | | VAEFDHPHILLSNPNSKFFSMVRETGESMT | |
| | | | RTLMEVAKAKYDSDNKEA | |
| 27 | Artificial | DNA | AGGTCCAACTGCAGGAGTCTGGGGGAGGCT | Bispecific |
| | | | CGGTGCAGTCTGGAGGGTCTCTGAGACTCT | VHH-GS |
| | | | CCTGTGCAGCCTCTGGATACACCATCACTA | linker |
| | | | ATAGTTACCGCATGGCCTGGTTCCGCCAGG | |
| | | | CTCCAGGGAAGGAGCGCGAGGGGGTCGCAG | |
| | | | CTATCAATAGTGGTGGTTCTACAACATACG | |
| | | | CAGACTCCGTGAAGGGCCGATTCACCATCT | |
| | | | CCCAAGACAACGCCCAGAACACGCTGTATT | |
| | | | TGCAGATGAACAGCCTGAAAGCCGAGGACA | |
| | | | CGGCCATGTATTACTGTGCGGAGGAGGAGG | |
| | | | AAGAGGAGGAGGAGGAGGCAGGGCGCGTAC | |
| | | | AGTGGTGGCCTGTACTACGCGCGCTGAACG | |
| | | | AGGATGATTATCTCTACTGGGGCCAGGGGA | |
| | | | CCCAGGTCACCGTCTCCTCAVCAGGTGCAG | |
| | | | CTGCAGGAGTCGGGGGGAGACTCGGTGCAG | |
| | | | GCTGGAGGGTCTCTGAGACTCGCCTGTGCN | |
| | | | GCCTCGGCCTCTGGCTACTCCGTGTGTGTG | |
| | | | GGGTGGGTGGGCTGGTTCCGCCCGGCTCCC | |
| | | | GGGCGGGAGCGCGGGGGGGTCGCCGTTGTT | |
| | | | TCTGTTCCTGGTGGTGGTTCCTTCTTTGGC | |
| | | | GGCGACGTGGGGGGCCGATTTTCCCTCTCC | |
| | | | CCGGACCACGCCCAGAACACGGTGTATCCG | |
| | | | CAAATGAACAGTCTGAAACCTGAGGACACT | |
| | | | GCCATGTACTATTGCGCAGCGCGCAATGCG | |
| | | | GGGGGGCGTTTTCGGCCTTCGGCCAATGGT | |
| | | | GGGTATAATTATTGGGGCCAGGGGACCCAG | |
| | | | GTCACCGTCTCCTCA | |
| 28 | Llama | Protein | QVQLQESGGRSVQSGGSLRLSCAASGIDVN | SEQ from |
| | glama | | RNAMGWFRQAPGTEREFVAGVRWSDAYTDY | FIG. 12* |
| | | | ADSVKGRFTISRDNNKNTVYLQMGSLEAGD | Bispecific |
| | | | TALYYCAAGLLDVQYVRQAAGYSYWGQGTQ | |
| | | | VTVSS | |
| 29 | Artificial | Protein | QVQLQESGGGSVQSGGSLRLSCAASGYTIT | VHH-GS |
| | | | NSYRMAWFRQAPGKEREGVAAINSGGSTTY | liVHHnker |
| | | | ADSVKGRFTISQDNAQNTLYLQMNSLKAED | |
| | | | TAMYYCAAGRVQWWPVLRALNEDDYLYWGQ | |
| | | | GTQVTVSSGGGGSGGGGQVQLQESGGDSVQ | |
| | | | AGGSLRLACAASASGYSVCVGWVGWFRPAP | |
| | | | GRERGGVAVVSVPGGGSFFGGDVGGRFSLS | |
| | | | PDHAQNTVYPQMNSLKPEDTAMYYCAARNA | |
| | | | GGRFRPSANGGYNYWGQGTQVTVSS | |
| 30 | Spodoptera | Protein | LPTLPWSVCQEEWGDCLPSDPDLDPVGTIT | Sodium- |
| | frugiperda | | NETKSSAELYFLKTVLQQKDGIEDGLG | dependent |
| | | | | nutrient |
| | | | | amino |
| | | | | acid |
| | | | | transporter |
| | | | | 1-like |
| | | | | antigen* |
| 31 | Spodoptera | Protein | MKMSLRLTLKTVKMSGETNNGFDASPEDGK | Sodium- |
| | frugiperda | | AAALNDKSLVNGANEKSPEKKDEPERAVWG | dependent |
| | | | NQIEFLMSCIATSVGLGNVWRFPFVAYQNG | nutrient |
| | | | GGAFLIPYIIVLLLIGKPMYYLECVLGQFS | amino |
| | | | SKNSVTVWSLSPAMKGAGYATALGCGYILS | acid |
| | | | YYVSIVALCLYYLAMSFLPTLPWSVCQEEW | transporter |
| | | | GDCLPSDPDLDPVGTITNETKSSAELYFLK | 1-like- |
| | | | TVLQQKDGIEDGLGLPIWYLVVCLFGSWFI | FL* |
| | | | IFVIVSRGVKSSGKAAYFLALFPYVVMLIL | |
| | | | LITTSILPGAGTGILFFLTPQWDKLIELDV | |
| | | | WYAAVTQVFFSLSVCTGAIIMFSSYNGFRQ | |
| | | | NVYRDAMIVTTLDTFTSLLSGFTIFGILGN | |
| | | | LAYELDKDVDDVTGSAGTGLAFISYPDAIS | |
| | | | KTFQPQLFAVLFFLMMTVLGIGSAVALLST | |
| | | | INTVMMDAFPRIKTIYMSAFCCTIGFAIGL | |
| | | | IYVTPGGQYILELVDYFGGTFLILFCAIAE | |
| | | | IIGVFWIYGLENLCLDIEYMLGVKTSFYWR | |
| | | | CCWGVIMPAMMITVFIYALATSETLKFGED | |
| | | | YYYPTAGYVAGYMMLFVGVAFVPISIGLTM | |
| | | | YKNKTGDCAETAKRSFRPKESWGPREEFER | |
| | | | LNWIEFRREAEAERAQKRTSWLQHIRYSLF | |
| | | | GGYRR | |
| 32 | Spodoptera | Protein | MGAMFRSEEMALCQLFIQPEAAYTSVSELG | V-ATPase |
| | frugiperda | | EAGSVQFRDLNPDVNAFQRKFVNEVRRCDE | subunit a- |
| | | | MERKLRYIEAEVHKDGVHIPAVKEAPRAPN | antigen* |
| | | | PREIIDLEAHLEKTENEILELSHNAVNLKQ | |
| | | | NYLELTELRHVLEKTEAFFIAQEEIGMDSM | |
| | | | TKSLISDETGQQAATRGRLGFVAGVVNRER | |
| | | | VPAFERMLWRISRGNVFLRRAELDKPLEDP | |
| | | | ATGNEIYKTVFVAFFQGEQLKSRIKKVCSG | |
| | | | FHASLYPCPPSNTERQDMVKGVRTRLEDLN | |
| | | | MVLNQTSDHRQRKALADGSNACGSSIPSFL | |
| | | | NCIETDEEPPTFNRTNRFTRGFQNLIDAYG | |
| | | | VASYRECNPALYTTITFPFLFA | |
| 33 | Spodoptera | Protein | MGAMFRSEEMALCQLFIQPEAAYTSVSELG | V-ATPase |
| | frugiperda | | EAGSVQFRDLNPDVNAFQRKFVNEVRRCDE | subunit a |
| | | | MERKLRYIEAEVHKDGVHIPAVKEAPRAPN | -full |
| | | | PREIIDLEAHLEKTENEILELSHNAVNLKQ | length* |
| | | | NYLELTELRHVLEKTEAFFIAQEEIGMDSM | |
| | | | TKSLISDETGQQAATRGRLGFVAGWVNRER | |
| | | | VPAFERMLWRISRGNVFLRRAELDKPLEDP | |
| | | | ATGNEIYKTVFVAFFQGEQLKSRIKKVCSG | |
| | | | FHASLYPCPPSNTERQDMVKGVRTRLEDLN | |
| | | | MVLNQTSDHRQRKALADGSNACGSSIPSFL | |
| | | | NCIETDEEPPTFNRTNRFTRGFQNLIDAYG | |
| | | | VASYRECNPALYTTITFPFLFAVMFGDLGH | |
| | | | GCIMALFGAWMVCNEVKLAAKKSNNEIWNI | |
| | | | FFAGRYIILLMGCFSMYTGLVYNDIFSKSM | |
| | | | NIFGSSWSVPYDNDTMEHNAALTMDPKTSY | |
| | | | NNNPYFIGIDPVWQSADNKIIFLNSYKMKL | |
| | | | SIIFGVLHMIFGVCMSVVNYNFFRRRYSIV | |
| | | | LEFLPQVIFLCLLFLYMVFMMFYKWVAYSA | |
| | | | FSEEQAYTPGCAPSVLILFINMMLFSSTAP | |
| | | | PEGCNEYMFEAQASIQRVFVLVALCCIPVM | |
| | | | LLGKPLYLLAAGKKKKEAKPEHSNGSVNPG | |
| | | | IEMQEQTDIGDGVPKPEAKPAASGHDHEDE | |
| | | | PFSEIMIHQAIHTIEYVLSTISHTASYLRL | |
| | | | WALSLAHAELSEVLWNMVLTFGLKDHDYIG | |
| | | | GIKLYVAFCFWALFTLAILVMMEGLSAFLH | |
| | | | TLRLHWVEFMSKFYSGLGYIFQPFCFKTIL | |
| | | | EQEDNKDD | |
| 34 | Spodoptera | Protein | MENNIQNQCVPYNCLNNPEVEILNEERSTG | Cry1Fa |
| | frugiperda | | RLPLDISLSLTRFLLSEFVPGVGVAFGLFD | domain II* |
| | | | LIWGFITPSDWSLFLLQIEQLIEQRIETLE | |
| | | | RNRAITTLRGLADSYEIYIEALREWEANPN | |
| | | | NAQLREDVRIRFANTDDALITAINNFTLTS | |
| | | | FEIPLLSVYVQAANLHLSLLRDAVSFGQGW | |
| | | | GLDIATVNNHYNRLINLIHRYTKHCLDTYN | |
| | | | QGLENLRGTNTRQWARFNQFRRDLTLTVLD | |
| | | | IVALFPNYDVRTYPIQTSSQLTREIYTSSV | |
| | | | IEDSPVSANIPNGFNRAEFGVRPPHLMDFM | |
| | | | NSLFVTAETVRSQTVWGGHLVSSRNTAGNR | |
| | | | INFPSYGVFNPGGAIWIADEDPRPFYRTLS | |
| | | | DPVFVRGGFGNPHYVLGLRGVAFQQTGTNH | |
| | | | TRTFRNSGTIDSLDEIPPQDNSGAPWNDYS | |
| | | | HVLNHVTFVRWPGEISGSDSWRAPMFSWTH | |
| | | | RSATPTNTIDPERITQIPLVKAHTLQSGTT | |
| | | | VVRGPGFTGGDILRRTSGGPFAYTIVNING | |
| | | | QLPQRYRARIRYASTTNLRIYVTVAGERIF | |
| | | | AGQFNKTMDTGDPLTFQSFSYATINTAFTF | |
| | | | PMSQSSFTVGADTFSSGNEVYIDRFELIPV | |
| | | | TATFEAEYDLERAQKAVNALFTSINQIGIK | |
| | | | TDVTDYHIDQVSNLVDCLSDEFCLDEKREL | |
| | | | SEKVKHAKRLSDERNLLQDPNFKGINRQLD | |
| | | | RGWRGSTDITIQRGDDVFKENYVTLPGTFD | |
| | | | ECYPTYLYQKIDESKLKPYTRYQLRGYIED | |
| | | | SQDLEIYLIRYNAKHETVNVLGTGSLWPLS | |
| | | | VQSPIRKCGEPNRCAPHLEWNPDLDCSCRD | |
| | | | GEKCAHHSHHFSLDIDVGCTDLNEDLDVWV | |
| | | | IFKIKTQDGHARLGNLEFLEEKPLVGEALA | |
| | | | RVKRAEKKWRDKREKLELETNIVYKEAKES | |
| | | | VDALFVNSQYDQLQADTNIAMIHAADKRVH | |
| | | | RIREAYLPELSVIPGVNVDIFEELKGRIFT | |
| | | | AFFLYDARNVIKNGDFNNGLSCWNVKGHVD | |
| | | | VEEQNNHRSVLVVPEWEAEVSQEVRVCPGR | |
| | | | GYILRVTAYKEGYGEGCVTIHEIENNTDEL | |
| | | | KFSNCVEEEVYPNNTVTCNDYTANQEEYGG | |
| | | | AYTSRNRGYDETYGSNSSVPADYASVYEEK | |
| | | | SYTDGRRDNPCESNRGYGDYTPLPAGYVTK | |
| | | | ELEYFPETDKVWIEIGETEGTFIVDSVELL | |
| | | | LMEE | |
| 35 | Spodoptera | Protein | GEFLDRLSATDEDGLHAGRVTFSIAGNDEA | Cadherin- |
| | frugiperda | | AEYFNVLNDGDNSAMLTLKQALPAGVQQFE | antigen* |
| | | | LVIRATDGGTEPGPRSTDCSVTVVFVMTQG | * |
| | | | DPVFDDNAASVRFVEKEAGMSEKFQLPQAD | |
| | | | DPKNYRCMDDCHTIYYSIVDGNDGDHFAVE | |
| | | | PETNVIYLLKPLDRSQQEQYRVVVAASNTP | |
| | | | GGTSTLSSSLLTVTIGVREANPRPIFESEF | |
| | | | YTAGVLHTDSIHKELVYLAAKHSEGLPIVY | |
| | | | SIDQETMKIDESLQTVVEDAFDINSATGVI | |
| | | | SLNFQPTSVMHGSFDFEVVASDTRGASDRA | |
| | | | KVSIYMISTRVRVAFLFYNTEAEVNERRNF | |
| | | | IAQTFANAFGMTCNIDSVLPATDANGVIRE | |
| | | | GYTELQAHFIRDDQPVPADYIEGLFTELNT | |
| | | | LRDIREVLSTQQLTLLDFAAGGSAVLPGGE | |
| | | | YALAVYI | |
| 36 | Artificial | Protein | GGGGSGGGG | linker |
| 37 | Spodoptera | Protein | SPESASTTPTPAPIVSPTTDSDGSSGTTEG | Venom |
| | frugiperda | | PTSPLTPSTTPSTTSTTTTTTTTTTTTTTT | dipeptidyl |
| | | | TTETPPDESPGQLDLEEIIDGVFSPPSFNA | peptidase |
| | | | TWATGSELMFRNDNGDLVLYDVDSDTPMPI | 4-like |
| | | | VSNTSKILQEASRVMQLSPEGKDVMLAHSV | isoform |
| | | | APVYRYSFIARYTAVNIENEEEVPITPPSV | X1- |
| | | | PREQALLQNLVWGPSGTSLAFVYYNNIYYQ | antigen* |
| | | | PSLKEAPRQITTDGVLNVIYNGIPDWVYEE | |
| | | | EVFGSNNAIWFSKDGTKMAYVTFDDSHVEV | |
| | | | MRVPHYGIPGETQYTRHRQIRYPKPNTTNP | |
| | | | TVKVTLWNLITDTPSVVEAPNDLNQPILKT | |
| | | | VKFINNDDIAVVWTNREQTSLRVQKCRASG | |
| | | | NTAECTTIYNYVENGGWIDNIPFFFNDAGN | |
| | | | SFITILPFAVDGVRFKQIVQVTEGTATAPA | |
| | | | TVKNRVNNPHTVLEILAWGTDDVIWYKATS | |
| | | | VSDSREQHIFSVNSQDVISCFTCNIRRTDG | |
| | | | GLCLYNEGTISTAGDRITINCAGPDVPQIF | |
| | | | IYKTNGELVRVWDEGADLSNLMHNRTLPVT | |
| | | | LRAQITSPLGQASTDIHIQAPADYAHRTNV | |
| | | | PLLVYVYGGPDTALVTRQWSLDWGSSLVSR | |
| | | | WGIAVAHIDGRGSGLRGVENMFALNRKLGT | |
| | | | VEIEDQIAGAKYRYIQDNFPWIDANRTCIW | |
| | | | GWSYGGYAASKALAEGGDVFRCAAAIAPVV | |
| | | | DWRFYVDTIYTERYMGLPTAEDNAEGYEVS | |
| | | | SLLTKAEALREKSYFLVHGTADDNVHYQHA | |
| | | | MLLSRLLQRRDVFFTQMSYTDEDHGLVGVR | |
| | | | PHLYHALERFLQEYML | |
| 38 | Spodoptera | Protein | MAQDNSNSTMEMGTSDQVLVATKRKKTIGY | Venom |
| | frugiperda | | IIGTIAMLAVGGVVIALIVVLSPESASTTP | dipeptidyl |
| | | | TPAPIVSPTTDSDGSSGTTEGPTSPLTPST | peptidase |
| | | | TPSTTSTTTTTTTTTTTTTTTTTETPPDES | 4-like |
| | | | PGQLDLEEIIDGVFSPPSFNATWATGSELM | isoform |
| | | | FRNDNGDLVLYDVDSDTPMPIVSNTSKILQ | X1-FL* |
| | | | EASRVMQLSPEGKDVMLAHSVAPVYRYSFI | |
| | | | ARYTAVNIENEEEVPITPPSVPREQALLQN | |
| | | | LVWGPSGTSLAFVYYNNIYYQPSLKEAPRQ | |
| | | | ITTDGVLNVIYNGIPDWVYEEEVFGSNNAI | |
| | | | WFSKDGTKMAYVTFDDSHVEVMRVPHYGIP | |
| | | | GETQYTRHRQIRYPKPNTTNPTVKVTLWNL | |
| | | | ITDTPSVVEAPNDLNQPILKTVKFINNDDI | |
| | | | AVVWTNREQTSLRVQKCRASGNTAECTTIY | |
| | | | NYVENGGWIDNIPFFFNDAGNSFITILPFA | |
| | | | VDGVRFKQIVQVTEGTATAPATVKNRVNNP | |
| | | | HTVLEILAWGTDDVIWYKATSVSDSREQHI | |
| | | | FSVNSQDVISCFTCNIRRTDGGLCLYNEGT | |
| | | | ISTAGDRITINCAGPDVPQIFIYKTNGELV | |
| | | | RVWDEGADLSNLMHNRTLPVTLRAQITSPL | |
| | | | GQASTDIHIQAPADYAHRTNVPLLVYVYGG | |
| | | | PDTALVTRQWSLDWGSSLVSRWGIAVAHID | |
| | | | GRGSGLRGVENMFALNRKLGTVEIEDQIAG | |
| | | | AKYRYIQDNFPWIDANRTCIWGWSYGGYAA | |
| | | | SKALAEGGDVFRCAAAIAPVVDWRFYVDTI | |
| | | | YTERYMGLPTAEDNAEGYEVSSLLTKAEAL | |
| | | | REKSYFLVHGTADDNVHYQHAMLLSRLLQR | |
| | | | RDVFFTQMSYTDEDHGLVGVRPHLYHALER | |
| | | | FLQEYML | |
| 39 | Spodoptera | | LPTYGTPVSEGLAQLRVYNGYNCNFTLNTA | Peptide |
| | frugiperda | | NLNTLEENATRDFEIGPLSAYENLHIFADD | transporter |
| | | | FVDLPYYLQGEPATECAGIAYSGYFNLKEK | family 1 |
| | | | TANSFFIKKDGLYNFTDNNDKAIDGVNVRF | isoform |
| | | | LSNINSVVDISIENDQKNKTLLSIQSNDTS | X1- |
| | | | QKSIAKGVSNVTVGGFVVLSDFNFKSGAVY | antigen |
| | | | TINIYEDRAGVYYANTVMITPSNSIHILW | |
| 40 | Spodoptera | | MEFTIVALLLIAFGTGGIKPCVSAFGGDQF | Peptide |
| | frugiperda | | KLPEQERYLGYFFSLFYFAINAGSLISTFL | transporter |
| | | | TPILRADVHCFGDNDCYSLAFGVPGILMVV | family 1 |
| | | | SIVFFVAGKRLYVIKKPAGNVLGKVSTCIG | isoform |
| | | | HAVVKSCKSKEKREHWLDHADDKYDSNLIE | X1-FL* |
| | | | DVKALLRVLVLFIPLPVFWALFDQQGSRWT | |
| | | | FQADRMEQDIGSWTLKADQMQVLNPLLILI | |
| | | | FIPIFEVAIYPFLTWCKLVRKPLHKMIWGG | |
| | | | ILAACAFIISGIVELNLLPTYGTPVSEGLA | |
| | | | QLRVYNGYNCNFTLNTANLNTLEENATRDF | |
| | | | EIGPLSAYENLHIFADDFVDLPYYLQGEPA | |
| | | | TECAGIAYSGYFNLKEKTANSFFIKKDGLY | |
| | | | NFTDNNDKAIDGVNVRFLSNINSVVDISIE | |
| | | | NDQKNKTLLSIQSNDTSQKSIAKGVSNVTV | |
| | | | GGFVVLSDFNFKSGAVYTINIYEDRAGVYY | |
| | | | ANTVMITPSNSIHILWLIPQYVVMTMGEVM | |
| | | | FSVTGLEFSFTQAPASMKSVLQSVWLLTVA | |
| | | | FGNLIVVLIVEGNFLDAQWKEFFLFAGLML | |
| | | | IDMLIFTTMAFRYKYKELSSSDENLAIEEI | |
| | | | KMPEKTSQDKHEKN | |
| 41 | Lama | DNA | GTCCTGGCTGCTCTTCTACAAGG | CH2 exon |
| | glama | | | llama |
| | | | | heavy |
| | | | | chain 5′ |
| | | | | primer |
| 42 | Lama | DNA | GGTACGTGCTGTTGAACTGTTCC | CH2 exon |
| | glama | | | llama |
| | | | | heavy |
| | | | | chain 3′ |
| | | | | primer |
| 43 | Spodoptera | Protein | MKDSKGNAWQLKPGEKENENKLPDPLNEDV | FAW |
| | frugiperda | | QKGQFSDTSSEPSEAKAEEVPSIPFITLFR | ABCC1- |
| | | | FASTRDKLFIICALICSAVAAISTPLNTLL | FL |
| | | | LAYLLEAMVNYSIFGDADAFMKSLLNFAIY | |
| | | | NAAVGAALVVLSYAATTLMNIAAYNQVYVI | |
| | | | RQEYLKAALNQDFGYFDVHKNAEIANKMNS | |
| | | | DIMKLEEGIGEKLATFFFYQASFLSSVIMA | |
| | | | LVKGWKLALLCLISFPVTMTLVGVAGLIAA | |
| | | | SLSKKEAIATGKAGAIAEEVISAIRTVYAF | |
| | | | SGQEKELDRYEGHLNDARKINVKKSLFNGL | |
| | | | AMGCLFFCIFCAYALSFWFGYRLMVTDGYD | |
| | | | VSTMIAVFFGVMTGSANFGISSTLMEVFGS | |
| | | | ARGAGAHIFNMIDNVPTINPLQNRGTVPSD | |
| | | | IEGNIELKNVEFHYPSRPDVPVLKGVSIKV | |
| | | | KRGQSVALVGHSGCGKSTIIQLISRFYDVV | |
| | | | EGSVAIDGNDVRDLSVRWLREQIGLVGQEP | |
| | | | VLFNTTVRENIRYGRENATNEEIEACARQA | |
| | | | NAHQFIMKLPKGYDTLVGERGASLSGGQKQ | |
| | | | RIAIARALVRNPKILLLDEATSALDTSSEA | |
| | | | KVQKALDKAQEGRTTIVVAHRLSTIRNVDV | |
| | | | IYVFKAGLVVECGNHTELMASKGHYYDMVM | |
| | | | LQNLPGVDEQSPEKTKLSRETSIISEKDDE | |
| | | | DEFLEFRNDVKEDAAEAPDISFMRVLKLNK | |
| | | | PEWKSVTLASICSLMSGFCMPLFAVIFGDF | |
| | | | LALLDGDDPDEIQKGVSRLALIFVGIGVFS | |
| | | | GITNFIVVFFYGIAGEALTKRLRLMMFRKL | |
| | | | LEMDIGFYDDKDNSTGALCARLSGEAAAVQ | |
| | | | GATGQRIGTVVQAVGTFGFALVLSLIFEWR | |
| | | | VGLVALTFVPIIIFVLYKEGRMTYAATSGT | |
| | | | VKVMETSSKIAVEAVANVRTVASLGREETF | |
| | | | RREYSKQLRPALDTAVRSSHWRGLVFGMSR | |
| | | | GVFNFVIASSLYYGGTIIVNEGVPFEQVFK | |
| | | | SAQALLMGATSAAQAFAFAPNFQKGIKAAG | |
| | | | RVIVLLGRQSKITDPAEPAVKNFNGTGEAS | |
| | | | LQGIQFRYPTRPLVRVLKDLNLEIQRGKTV | |
| | | | ALVGASGCGKSTVIQLLERYYDPEDGIVAQ | |
| | | | DGVPLPKLNLVDARRAIGFVQQEPILFDRT | |
| | | | IGENIAYGNNEARVSNDEVIEAAQQANIHN | |
| | | | FITSLPLGYDTNIGSKGTQLSGGQKQRVAI | |
| | | | ARALIRRPKMLLLDEATSALDTESEKVVQE | |
| | | | ALDKAKAGRTCVMIAHRLSTVRDADVICVI | |
| | | | HEGQVAEMGTHNELLELKGLYYNLNRRGYA | |
| 44 | Spodoptera | Protein | YLLEAMVNYSIFGDADAFMKSLLNF | FAW |
| | frugiperda | | | ABCC1- |
| | | | | antigen |
| 45 | Bacillus | DNA | TTACAATTCAAGATGAATTGCAGGTAAATG | Cry1Ac |
| | thuringiensis | | GTTCTAACATGTATAAGTGTAAGTATTTCT | CDS |
| | | | ACATTACCACAAATTCTCAATTTGTATATG | |
| | | | TAAAATAGGAAAAGTGGATTTTATATATAA | |
| | | | GTATAAAAAGTAATAAGACTTTAAAATAAG | |
| | | | TTAACGGAATACAAACCCTTAATGCATTGG | |
| | | | TTAAACATTGTAAAGTCTAAAGCATGGATA | |
| | | | ATGGGCGAGAAGTAAGTAGATTGTTAACAC | |
| | | | CCTGGGTCAAAAATTGATATTTAGTAAAAT | |
| | | | TAGTTGCACTTTGTGCATTTTTTCATAAGA | |
| | | | TGAGTCATATGTTTTAAATTGTAGTAATGA | |
| | | | AAAACAGTATTATATCATAATGAATTGGTA | |
| | | | TCTTAATAAAAGAGATGGAGGTAACTTATG | |
| | | | GATAACAATCCGAACATCAATGAATGCATT | |
| | | | CCTTATAATTGTTTAAGTAACCCTGAAGTA | |
| | | | GAAGTATTAGGTGGAGAAAGAATAGAAACT | |
| | | | GGTTACACCCCAATCGATATTTCCTTGTCG | |
| | | | CTAACGCAATTTCTTTTGAGTGAATTTGTT | |
| | | | CCCGGTGCTGGATTTGTGTTAGGACTAGTT | |
| | | | GATATAATATGGGGAATTTTTGGTCCCTCT | |
| | | | CAATGGGACGCATTTCTTGTACAAATTGAA | |
| | | | CAGTTAATTAACCAAAGAATAGAAGAATTC | |
| | | | GCTAGGAACCAAGCCATTTCTAGATTAGAA | |
| | | | GGACTAAGCAATCTTTATCAAATTTACGCA | |
| | | | GAATCTTTTAGAGAGTGGGAAGCAGATCCT | |
| | | | ACTAATCCAGCATTAAGAGAAGAGATGCGT | |
| | | | ATTCAATTCAATGACATGAACAGTGCCCTT | |
| | | | ACAACCGCTATTCCTCTTTTTGCAGTTCAA | |
| | | | AATTATCAAGTTCCTCTTTTATCAGTATAT | |
| | | | GTTCAAGCTGCAAATTTACATTTATCAGTT | |
| | | | TTGAGAGATGTTTCAGTGTTTGGACAAAGG | |
| | | | TGGGGATTTGATGCCGCGACTATCAATAGT | |
| | | | CGTTATAATGATTTAACTAGGCTTATTGGC | |
| | | | AACTATACAGATTATGCTGTACGCTGGTAC | |
| | | | AATACGGGATTAGAACGTGTATGGGGACCG | |
| | | | GATTCTAGAGATTGGGTAAGGTATAATCAA | |
| | | | TTTAGAAGAGAATTAACACTAACTGTATTA | |
| | | | GATATCGTTGCTCTGTTCCCGAATTATGAT | |
| | | | AGTAGAAGATATCCAATTCGAACAGTTTCC | |
| | | | CAATTAACAAGAGAAATTTATACAAACCCA | |
| | | | GTATTAGAAAATTTTGATGGTAGTTTTCGA | |
| | | | GGCTCGGCTCAGGGCATAGAAAGAAGTATT | |
| | | | AGGAGTCCACATTTGATGGATATACTTAAC | |
| | | | AGTATAACCATCTATACGGATGCTCATAGG | |
| | | | GGTTATTATTATTGGTCAGGGCATCAAATA | |
| | | | ATGGCTTCTCCTGTCGGTTTTTCGGGGCCA | |
| | | | GAATTCACGTTTCCGCTATATGGAACCATG | |
| | | | GGAAATGCAGCTCCACAACAACGTATTGTT | |
| | | | GCTCAACTAGGTCAGGGCGTGTATAGAACA | |
| | | | TTATCGTCCACTTTATATAGAAGACCTTTT | |
| | | | AATATAGGGATAAATAATCAACAACTATCT | |
| | | | GTTCTTGACGGGACAGAATTTGCTTATGGA | |
| | | | ACCTCCTCAAATTTGCCATCCGCTGTATAC | |
| | | | AGAAAAAGCGGAACGGTAGATTCGCTGGAT | |
| | | | GAAATACCGCCACAGAATAACAACGTGCCA | |
| | | | CCTAGGCAAGGATTTAGTCATCGATTAAGC | |
| | | | CATGTTTCAATGTTTCGTTCAGGCTTTAGT | |
| | | | AATAGTAGTGTAAGTATAATAAGAGCTCCT | |
| | | | ATGTTCTCTTGGATACATCGTAGTGCTGAA | |
| | | | TTTAATAATATAATTGCATCGGATAGTATT | |
| | | | ACTCAAATCCCTGCAGTGAAGGGAAACTTT | |
| | | | CTTTTTAATGGTTCTGTAATTTCAGGACCA | |
| | | | GGATTTACTGGTGGGGACTTAGTTAGATTA | |
| | | | AATAGTAGTGGAAATAACATTCAGAATAGA | |
| | | | GGGTATATTGAAGTTCCAATTCACTTCCCA | |
| | | | TCGACATCTACCAGATATCGAGTTCGTGTA | |
| | | | CGGTATGCTTCTGTAACCCCGATTCACCTC | |
| | | | AACGTTAATTGGGGTAATTCATCCATTTTT | |
| | | | TCCAATACAGTACCAGCTACAGCTACGTCA | |
| | | | TTAGATAATCTACAATCAAGTGATTTTGGT | |
| | | | TATTTTGAAAGTGCCAATGCTTTTACATCT | |
| | | | TCATTAGGTAATATAGTAGGTGTTAGAAAT | |
| | | | TTTAGTGGGACTGCAGGAGTGATAATAGAC | |
| | | | AGATTTGAATTTATTCCAGTTACTGCAACA | |
| | | | CTCGAGGCTGAATATAATCTGGAAAGAGCG | |
| | | | CAGAAGGCGGTGAATGCGCTGTTTACGTCT | |
| | | | ACAAACCAACTAGGGCTAAAAACAAATGTA | |
| | | | ACGGATTATCATATTGATCAAGTGTCCAAT | |
| | | | TTAGTTACGTATTTATCGGATGAATTTTGT | |
| | | | CTGGATGAAAAGCGAGAATTGTCCGAGAAA | |
| | | | GTCAAACATGCGAAGCGACTCAGTGATGAA | |
| | | | CGCAATTTACTCCAAGATTCAAATTTCAAA | |
| | | | GACATTAATAGGCAACCAGAACGTGGGTGG | |
| | | | GGCGGAAGTACAGGGATTACCATCCAAGGA | |
| | | | GGGGATGACGTATTTAAAGAAAATTACGTC | |
| | | | ACACTATCAGGTACCTTTGATGAGTGCTAT | |
| | | | CCAACATATTTGTATCAAAAAATCGATGAA | |
| | | | TCAAAATTAAAAGCCTTTACCCGTTATCAA | |
| | | | TTAAGAGGGTATATCGAAGATAGTCAAGAC | |
| | | | TTAGAAATCTATTTAATTCGCTACAATGCA | |
| | | | AAACATGAAACAGTAAATGTGCCAGGTACG | |
| | | | GGTTCCTTATGGCCGCTTTCAGCCCAAAGT | |
| | | | CCAATCGGAAAGTGTGGAGAGCCGAATCGA | |
| | | | TGCGCGCCACACCTTGAATGGAATCCTGAC | |
| | | | TTAGATTGTTCGTGTAGGGATGGAGAAAAG | |
| | | | TGTGCCCATCATTCGCATCATTTCTCCTTA | |
| | | | GACATTGATGTAGGATGTACAGACTTAAAT | |
| | | | GAGGACCTAGGTGTATGGGTGATCTTTAAG | |
| | | | ATTAAGACGCAAGATGGGCACGCAAGACTA | |
| | | | GGGAATCTAGAGTTTCTCGAAGAGAAACCA | |
| | | | TTAGTAGGAGAAGCGCTAGCTCGTGTGAAA | |
| | | | AGAGCGGAGAAAAAATGGAGAGACAAACGT | |
| | | | GAAAAATTGGAATGGGAAACAAATATCGTT | |
| | | | TATAAAGAGGCAAAAGAATCTGTAGATGCT | |
| | | | TTATTTGTAAACTCTCAATATGATCAATTA | |
| | | | CAAGCGGATACGAATATTGCCATGATTCAT | |
| | | | GCGGCAGATAAACGTGTTCATAGCATTCGA | |
| | | | GAAGCTTATCTGCCTGAGCTGTCTGTGATT | |
| | | | CCGGGTGTCAATGCGGCTATTTTTGAAGAA | |
| | | | TTAGAAGGGCGTATTTTCACTGCATTCTCC | |
| | | | CTATATGATGCGAGAAATGTCATTAAAAAT | |
| | | | GGTGATTTTAATAATGGCTTATCCTGCTGG | |
| | | | AACGTGAAAGGGCATGTAGATGTAGAAGAA | |
| | | | CAAAACAACCAACGTTCGGTCCTTGTTGTT | |
| | | | CCGGAATGGGAAGCAGAAGTGTCACAAGAA | |
| | | | GTTCGTGTCTGTCCGGGTCGTGGCTATATC | |
| | | | CTTCGTGTCACAGCGTACAAGGAGGGATAT | |
| | | | GGAGAAGGTTGCGTAACCATTCATGAGATC | |
| | | | GAGAACAATACAGACGAACTGAAGTTTAGC | |
| | | | AACTGCGTAGAAGAGGAAATCTATCCAAAT | |
| | | | AACACGGTAACGTGTAATGATTATACTGTA | |
| | | | AATCAAGAAGAATACGGAGGTGCGTACACT | |
| | | | TCTCGTAATCGAGGATATAACGAAGCTCCT | |
| | | | TCCGTACCAGCTGATTATGCGTCAGTCTAT | |
| | | | GAAGAAAAATCGTATACAGATGGACGAAGA | |
| | | | GAGAATCCTTGTGAATTTAACAGAGGGTAT | |
| | | | AGGGATTACACGCCACTACCAGTTGGTTAT | |
| | | | GTGACAAAAGAATTAGAATACTTCCCAGAA | |
| | | | ACCGATAAGGTATGGATTGAGATTGGAGAA | |
| | | | ACGGAAGGAACATTTATCGTGGACAGCGTG | |
| | | | GAATTACTCCTTATGGAGGAATAGTCTCAT | |
| | | | GCAAACTCAGGTTTAAATATCGTTTTCAAA | |
| | | | TCAATTGTCCAAGAGCAGCATTACAAATAG | |
| | | | ATAAGTAATTTGTTGTAATGAAAAACGGAC | |
| | | | ATCACCTCCATTGAAACGGAGTGATGTCCG | |
| | | | TTTTACTATGTTATTTTCTAGTAATACATA | |
| | | | TGTATAGAGCAACTTAATCAAGCAGAGATA | |
| | | | TTTTCACCTATCGATGAAAATATCTCTGCT | |
| | | | TTTTCTTTTTTTATTTGGTATATGCTTTAC | |
| | | | TTGTAATC | |
| 46 | Bacillus | | AAGCTTAATTAAAGATAATATCTTTGAATT | Cry3Aa |
| | thuringiensis | | GTAACGCCCCTCAAAAGTAAGAACTACAAA | |
| | | | AAAAGAATACGTTATATAGAAATATGTTTG | |
| | | | AACCTTCTTCAGATTACAAATATATTCGGA | |
| | | | CGGACTCTACCTCAAATGCTTATCTAACTA | |
| | | | TAGAATGACATACAAGCACAACCTTGAAAA | |
| | | | TTTGAAAATATAACTACCAATGAACTTGTT | |
| | | | CATGTGAATTATCGCTGTATTTAATTTTCT | |
| | | | CAATTCAATATATAATATGCCAATACATTG | |
| | | | TTACAAGTAGAAATTAAGACACCCTTGATA | |
| | | | GCCTTACTATACCTAACATGATGTAGTATT | |
| | | | AAATGAATATGTAAATATATTTATGATAAG | |
| | | | AAGCGACTTATTTATAATCATTACATATTT | |
| | | | TTCTATTGGAATGATTAAGATTCCAATAGA | |
| | | | ATAGTGTATAAATTATTTATCTTGAAAGGA | |
| | | | GGGATGCCTAAAAACGAAGAACATTAAAAA | |
| | | | CATATATTTGCACCGTCTAATGGATTTATG | |
| | | | AAAAATCATTTTATCAGTTTGAAAATTATG | |
| | | | TATTATGATAAGAAAGGGAGGAAGAAAAAT | |
| | | | GAATCCGAACAATCGAAGTGAACATGATAC | |
| | | | AATAAAAACTACTGAAAATAATGAGGTGCC | |
| | | | AACTAACCATGTTCAATATCCTTTAGCGGA | |
| | | | AACTCCAAATCCAACACTAGAAGATTTAAA | |
| | | | TTATAAAGAGTTTTTAAGAATGACTGCAGA | |
| | | | TAATAATACGGAAGCACTAGATAGCTCTAC | |
| | | | AACAAAAGATGTCATTCAAAAAGGCATTTC | |
| | | | CGTAGTAGGTGATCTCCTAGGCGTAGTAGG | |
| | | | TTTCCCGTTTGGTGGAGCGCTTGTTTCGTT | |
| | | | TTATACAAACTTTTTAAATACTATTTGGCC | |
| | | | AAGTGAAGACCCGTGGAAGGCTTTTATGGA | |
| | | | ACAAGTAGAAGCATTGATGGATCAGAAAAT | |
| | | | AGCTGATTATGCAAAAAATAAAGCTCTTGC | |
| | | | AGAGTTACAGGGCCTTCAAAATAATGTCGA | |
| | | | AGATTATGTGAGTGCATTGAGTTCATGGCA | |
| | | | AAAAAATCCTGTGAGTTCACGAAATCCACA | |
| | | | TAGCCAGGGGCGGATAAGAGAGCTGTTTTC | |
| | | | TCAAGCAGAAAGTCATTTTCGTAATTCAAT | |
| | | | GCCTTCGTTTGCAATTTCTGGATACGAGGT | |
| | | | TCTATTTCTAACAACATATGCACAAGCTGC | |
| | | | CAACACACATTTATTTTTACTAAAAGACGC | |
| | | | TCAAATTTATGGAGAAGAATGGGGATACGA | |
| | | | AAAAGAAGATATTGCTGAATTTTATAAAAG | |
| | | | ACAACTAAAACTTACGCAAGAATATACTGA | |
| | | | CCATTGTGTCAAATGGTATAATGTTGGATT | |
| | | | AGATAAATTAAGAGGTTCATCTTATGAATC | |
| | | | TTGGGTAAACTTTAACCGTTATCGCAGAGA | |
| | | | GATGACATTAACAGTATTAGATTTAATTGC | |
| | | | ACTATTTCCATTGTATGATGTTCGGCTATA | |
| | | | CCCAAAAGAAGTTAAAACCGAATTAACAAG | |
| | | | AGACGTTTTAACAGATCCAATTGTCGGAGT | |
| | | | CAACAACCTTAGGGGCTATGGAACAACCTT | |
| | | | CTCTAATATAGAAAATTATATTCGAAAACC | |
| | | | ACATCTATTTGACTATCTGCATAGAATTCA | |
| | | | ATTTCACACGCGGTTCCAACCAGGATATTA | |
| | | | TGGAAATGACTCTTTCAATTATTGGTCCGG | |
| | | | TAATTATGTTTCAACTAGACCAAGCATAGG | |
| | | | ATCAAATGATATAATCACATCTCCATTCTA | |
| | | | TGGAAATAAATCCAGTGAACCTGTACAAAA | |
| | | | TTTAGAATTTAATGGAGAAAAAGTCTATAG | |
| | | | AGCCGTAGCAAATACAAATCTTGCGGTCTG | |
| | | | GCCGTCCGCTGTATATTCAGGTGTTACAAA | |
| | | | AGTGGAATTTAGCCAATATAATGATCAAAC | |
| | | | AGATGAAGCAAGTACACAAACGTACGACTC | |
| | | | AAAAAGAAATGTTGGCGCGGTCAGCTGGGA | |
| | | | TTCTATCGATCAATTGCCTCCAGAAACAAC | |
| | | | AGATGAACCTCTAGAAAAGGGATATAGCCA | |
| | | | TCAACTCAATTATGTAATGTGCTTTTTAAT | |
| | | | GCAGGGTAGTAGAGGAACAATCCCAGTGTT | |
| | | | AACTTGGACACATAAAAGTGTAGACTTTTT | |
| | | | TAACATGATTGATTCGAAAAAAATTACACA | |
| | | | ACTTCCGTTAGTAAAGGCATATAAGTTACA | |
| | | | ATCTGGTGCTTCCGTTGTCGCAGGTCCTAG | |
| | | | GTTTACAGGAGGAGATATCATTCAATGCAC | |
| | | | AGAAAATGGAAGTGCGGCAACTATTTACGT | |
| | | | TACACCGGATGTGTCGTACTCTCAAAAATA | |
| | | | TCGAGCTAGAATTCATTATGCTTCTACATC | |
| | | | TCAGATAACATTTACACTCAGTTTAGACGG | |
| | | | GGCACCATTTAATCAATACTATTTCGATAA | |
| | | | AACGATAAATAAAGGAGACACATTAACGTA | |
| | | | TAATTCATTTAATTTAGCAAGTTTCAGCAC | |
| | | | ACCATTCGAATTATCAGGGAATAACTTACA | |
| | | | AATAGGCGTCACAGGATTAAGTGCTGGAGA | |
| | | | TAAAGTTTATATAGACAAAATTGAATTTAT | |
| | | | TCCAGTGAATTAAATTAACTAGAAAGTAAA | |
| | | | GAAGTAGTGACCATCTATGATAGTAAGCAA | |
| | | | AGGATAAAAAAATGAGTTCATAAAATGAAT | |
| | | | AACATAGTGTTCTTCAACTTTCGCTTTTTG | |
| | | | AAGGTAGATGAAGAACACTATTTTTATTTT | |
| | | | CAAAATGAAGGAAGTTTTAAATATGTAATC | |
| | | | ATTTAAAGGGAACAATGAAAGTAGGAAATA | |
| | | | AGTCATTATCTATAACAAAATAACATTTTT | |
| | | | ATATAGCCAGAAATGAATTATAATATTAAT | |
| | | | CTTTTCTAAATTGACGTTTTTCTAAACGTT | |
| | | | CTATAGCTTCAAGACGCTTAGAATCATCAA | |
| | | | TATTTGTATACAGAGCTGTTGTTTCCATCG | |
| | | | AGTTATGTCCCATTTGATTCGCTAATAGAA | |
| | | | CAAGATCTTTATTTTCGTTATAATGATTGG | |
| | | | TTGCATAAGTATGGCGTAATTTATGAGGGC | |
| | | | TTTTCTTTTCATCAAAAGCCCTCGTGTATT | |
| | | | TCTCTGTAAGCTT | |
| 47 | Bacillus | DNA | ATGAACAAGAATAATACTAAATTAAGCACA | VIP3Aa |
| | thuringiensis | | AGAGCCTTACCAAGTTTTATTGATTATTTT | from HD-1 |
| | | | AATGGCATTTATGGATTTGCCACTGGTATC | |
| | | | AAAGACATTATGAACATGATTTTTAAAACG | |
| | | | GATACAGGTGGTGATCTAACCCTAGACGAA | |
| | | | ATTTTAAAGAATCAGCAGTTACTAAATGAT | |
| | | | ATTTCTGGTAAATTGGATGGGGTGAATGGA | |
| | | | AGCTTAAATGATCTTATCGCACAGGGAAAC | |
| | | | TTAAATACAGAATTATCTAAGGAAATATTA | |
| | | | AAAATTGCAAATGAACAAAATCAAGTTTTA | |
| | | | AATGATGTTAATAACAAACTCGATGCGATA | |
| | | | AATACGATGCTTCGGGTATATCTACCTAAA | |
| | | | ATTACCTCTATGTTGAGTGATGTAATGAAA | |
| | | | CAAAATTATGCGCTAAGTCTGCAAATAGAA | |
| | | | TACTTAAGTAAACAATTGCAAGAGATTTCT | |
| | | | GATAAGTTGGATATTATTAATGTAAATGTA | |
| | | | CTTATTAACTCTACACTTACTGAAATTACA | |
| | | | CCTGCGTATCAAAGGATTAAATATGTGAAC | |
| | | | GAAAAATTTGAGGAATTAACTTTTGCTACA | |
| | | | GAAACTAGTTCAAAAGTAAAAAAGGATGGC | |
| | | | TCTCCTGCAGATATTCTTGATGAGTTAACT | |
| | | | GAGTTAACTGAACTAGCGAAAAGTGTAACA | |
| | | | AAAAATGATGTGGATGGTTTTGAATTTTAC | |
| | | | CTTAATACATTCCACGATGTAATGGTAGGA | |
| | | | AATAATTTATTCGGGCGTTCAGCTTTAAAA | |
| | | | ACTGCATCGGAATTAATTACTAAAGAAAAT | |
| | | | GTGAAAACAAGTGGCAGTGAGGTCGGAAAT | |
| | | | GTTTATAACTTCTTAATTGTATTAACAGCT | |
| | | | CTGCAAGCAAAAGCTTTTCTTACTTTAACA | |
| | | | ACATGCCGAAAATTATTAGGCTTAGCAGAT | |
| | | | ATTGATTATACTTCTATTATGAATGAACAT | |
| | | | TTAAATAAGGAAAAAGAGGAATTTAGAGTA | |
| | | | AACATCCTCCCTACACTTTCTAATACTTTT | |
| | | | TCTAATCCTAATTATGCAAAAGTTAAAGGA | |
| | | | AGTGATGAAGATGCAAAGATGATTGTGGAA | |
| | | | GCTAAACCAGGACATGCATTGATTGGGTTT | |
| | | | GAAATTAGTAATGATTCAATTACAGTATTA | |
| | | | AAAGTATATGAGGCTAAGCTAAAACAAAAT | |
| | | | TATCAAGTCGATAAGGATTCCTTATCGGAA | |
| | | | GTTATTTATGGTGATATGGATAAATTATTG | |
| | | | TGCCCAGATCAATCTGAACAAATCTATTAT | |
| | | | ACAAATAACATAGTATTTCCAAATGAATAT | |
| | | | GTAATTACTAAAATTGATTTCACTAAAAAA | |
| | | | ATGAAAACTTTAAGATATGAGGTAACAGCG | |
| | | | AATTTTTATGATTCTTCTACAGGAGAAATT | |
| | | | GACTTAAATAAGAAAAAAGTAGAATCAAGT | |
| | | | GAAGCGGAGTATAGAACGTTAAGTGCTAAT | |
| | | | GATGATGGGGTGTATATGCCGTTAGGTGTC | |
| | | | ATCAGTGAAACATTTTTGACTCCGATTAAT | |
| | | | GGGTTTGGCCTCCAAGCTGATGAAAATTCA | |
| | | | AGATTAATTACTTTAACATGTAAATCATAT | |
| | | | TTAAGAGAACTACTGCTAGCAACAGACTTA | |
| | | | AGCAATAAAGAAACTAAATTGATCGTCCCG | |
| | | | CCAAGTGGTTTTATTAGCAATATTGTAGAG | |
| | | | AACGGGTCCATAGAAGAGGACAATTTAGAG | |
| | | | CCGTGGAAAGCAAATAATAAGAATGCGTAT | |
| | | | GTAGATCATACAGGCGGAGTGAATGGAACT | |
| | | | AAAGCTTTATATGTTCATAAGGACGGAGGA | |
| | | | ATTTCACAATTTATTGGAGATAAGTTAAAA | |
| | | | CCGAAAACTGAGTATGTAATCCAATATACT | |
| | | | GTTAAAGGAAAACCTTCTATTCATTTAAAA | |
| | | | GATGAAAATACTGGATATATTCATTATGAA | |
| | | | GATACAAATAATAATTTAGAAGATTATCAA | |
| | | | ACTATTAATAAACGTTTTACTACAGGAACT | |
| | | | GATTTAAAGGGAGTGTATTTAATTTTAAAA | |
| | | | AGTCAAAATGGAGATGAAGCTTGGGGAGAT | |
| | | | AACTTTATTATTTTGGAAATTAGTCCTTCT | |
| | | | GAAAAGTTATTAAGTCCAGAATTAATTAAT | |
| | | | ACAAATAATTGGACGAGTACGGGATCAACT | |
| | | | AATATTAGCGGTAATACACTCACTCTTTAT | |
| | | | CAGGGAGGACGAGGGATTCTAAAACAAAAC | |
| | | | CTTCAATTAGATAGTTTTTCAACTTATAGA | |
| | | | GTGTATTTTTCTGTGTCCGGAGATGCTAAT | |
| | | | GTAAGGATTAGAAATTCTAGGGAAGTGTTA | |
| | | | TTTGAAAAAAGATATATGAGCGGTGCTAAA | |
| | | | GATGTTTCTGAAATGTTCACTACAAAATTT | |
| | | | GAGAAAGATAACTTTTATATAGAGCTTTCT | |
| | | | CAAGGGAATAATTTATATGGTGGTCCTATT | |
| | | | GTACATTTTTACGATGTCTCTATTAAGTAA | |
| 48 | Artificial | Protein | MSPILGYWKIKGLVQPTRLLLEYLEEKYEE | NAAT |
| | | | HLYERDEGDKWRNKKFELGLEFPNLPYYID | antigen |
| | | | GDVKLTQSMAIIRYIADKHNMLGGCPKERA | peptide |
| | | | EISMLEGAVLDIRYGVSRIAYSKDFETLKV | construct* |
| | | | DFLSKLPEMLKMFEDRLCHKTYLNGDHVTH | |
| | | | PDFMLYDALDVVLYMDPMCLDAFPKLVCFK | |
| | | | KRIEAIPQIDKYLKSSKYIAWPLQGWQATF | |
| | | | GGGDHPPKSDGSGSHHHHHHGSGSLEVLFQ | |
| | | | GPLPTLPWSVCQEEWGDCLPSDPDLDPVGT | |
| | | | ITNETKSSAELYFLKTVLQQKDGIEDGLG | |
| 49 | Artificial | Protein | MGSGSHHHHHHGSGSNLYFQGGEFLDRLSA | Cadherin |
| | | | TDEDGLHAGRVTFSIAGNDEAAEYFNVLND | antigen |
| | | | GDNSAMLTLKQALPAGVQQFELVIRATDGG | peptide |
| | | | TEPGPRSTDCSVTVVFVMTQGDPVFDDNAA | construct |
| | | | SVRFVEKEAGMSEKFQLPQADDPKNYRCMD | |
| | | | DCHTIYYSIVDGNDGDHFAVEPETNVIYLL | |
| | | | KPLDRSQQEQYRVVVAASNTPGGTSTLSSS | |
| | | | LLTVTIGVREANPRPIFESEFYTAGVLHTD | |
| | | | SIHKELVYLAAKHSEGLPIVYSIDQETMKI | |
| | | | DESLQTVVEDAFDINSATGVISLNFQPTSV | |
| | | | MHGSFDFEVVASDTRGASDRAKVSIYMIST | |
| | | | RVRVAFLFYNTEAEVNERRNFIAQTFANAF | |
| | | | GMTCNIDSVLPATDANGVIREGYTELQAHF | |
| | | | IRDDQPVPADYIEGLFTELNTLRDIREVLS | |
| | | | TQQLTLLDFAAGGSAVLPGGEYALAVYI** | |
| | | | MNNVLNSGRTTICDAYNVVAHDPFSFEHKS | |
| | | | LDTIQKEWMEWKRTDHSLYVAPVVGTVSSF | |
| | | | LLKKVGSLIGKRILSELWGIIFPSGSTNLM | |
| | | | QDILRETEQFLNQRL | |
| 50 | Artificial | Protein | NTDTLARVNAELIGLQANIREFNQQVDNFL | cry2Aa/ |
| | | | NPTQNPVPLSITSSVNTMQQLFLNRLPQFQ | cry1F/ |
| | | | IQGYQLLLLPLFAQAANMHLSFIRDVILNA | cry2A |
| | | | DEWGISAATLRTYRDYLRNYTRDYSNYCIN | a chimera |
| | | | TYQTAFRGLNTRLHDMLEFRTYMFLNVFEY | toxin* |
| | | | VSIWSLFKYQSLMVSSGRTYPIQTSSQLTR | |
| | | | EIYTSSVIEDSPVSANIPNGFNRAEFGVRP | |
| | | | PHLMDFMNSLFVTAETVRSQTVWGGHLVSS | |
| | | | RNTAGNRINFPSYGVFNPGGAIWIADEDPR | |
| | | | PFYRTLSDPVFVRGGFGNPHYVLGLRGVAF | |
| | | | QQTGTNHTRTFRNSGTIDSLDEIPPQDNSG | |
| | | | APWNDYSHVLNHVTFVRWPGEISGSDSWRA | |
| | | | PMFSWTHRSATPTNTIEIYAANENGTMIHL | |
| | | | APEDYTGFTISPIHATQVNNQTRTFISEKF | |
| | | | GNQGDSLRFEQSNTTARYTLRGNGNSYNLY | |
| | | | LRVSSIGNSTIRVTINGRVYTVSNVNTTTN | |
| | | | NDGVNDNGARFSDINIGNIVASDNTNVTLD | |
| | | | INVTLNSGTPFDLMNIMFVPTNLPPLY | |
| 51 | Bacillus | Protein | MDNNPNINECIPYNCLSNPEVEVLGGERIE | Cry1Ac |
| | thuingiensis | | TGYTPIDISLSLTQFLLSEFVPGAGFVLGL | |
| | | | VDIIWGIFGPSQWDAFLVQIEQLINQRIEE | |
| | | | FARNQAISRLEGLSNLYQIYAESFREWEAD | |
| | | | PTNPALREEMRIQFNDMNSALTTAIPLLAV | |
| | | | QNYQVPLLSVYVQAANLHLSVLRDVSVFGQ | |
| | | | RWGFDAATINSRYNDLTRLIGNYTDYAVRW | |
| | | | YNTGLERVWGPDSRDWVRYNQFRRELTLTV | |
| | | | LDIVALFSNYDSRRYPIRTVSQLTREIYTN | |
| | | | PVLENFDGSFRGMAQRIEQNIRQPHLMDIL | |
| | | | NSITIYTDVHRGFNYWSGHQITASPVGFSG | |
| | | | PEFAFPLFGNAGNAAPPVLVSLTGLGIFRT | |
| | | | LSSPLYRRIILGSGPNNQELFVLDG | |
| 52 | Bacillus | | TEFSFASLTTNLPSTIYRQRGTVDSLDVIP | Cry3Aa |
| | thuingiensis | | PQDNSVPPRAGFSHRLSHVTMLSQAAGAVY | |
| | | | TLRAPTFSWQHRSAEFNNIIPSSQITQIPL | |
| | | | TKSTNLGSGTSVVKGPGFTGGDILRRTSPG | |
| | | | QISTLRVNITAPLSQRYRVRIRYASTTNLQ | |
| | | | FHTSIDGRPINQGNFSATMSSGSNLQSGSF | |
| | | | RTVGFTTPFNFSNGSSVFTLSAHVFNSGNE | |
| | | | VYIDRIEFVPAEVTFEAEYDLERAQKAVNE | |
| | | | LFTSSNQIGLKTDVTDYHIDQVSNLVECLS | |
| | | | DEFCLDEKQELSEKVKHAKRLSDERNLLQD | |
| | | | PNFRGINRQLDRGWRGSTDITIQGGDDVFK | |
| | | | ENYVTLLGTFDECYPTYLYQKIDESKLKAY | |
| | | | TRYQLRGYIEDSQDLEIYLIRYNAKHETVN | |
| | | | VPGTGSLWPLSAQSPIGKCGEPNRCAPHLE | |
| | | | WNPDLDCSCRDGEKCAHHSHHFSLDIDVGC | |
| | | | TDLNEDLGVWVIFKIKTQDGHARLGNLEFL | |
| | | | EEKPLVGEALARVKRAEKKWRDKREKLEWE | |
| | | | TNIVYKEAKESVDALFVNSQYDQLQADTNI | |
| | | | AMIHAADKRVHSIREAYLPELSVIPGVNAA | |
| | | | IFEELEGRIFTAFSLYDARNVIKNGDFNNG | |
| | | | LSCWNVKGHVDVEEQNNQRSVLVVPEWEAE | |
| | | | VSQEVRVCPGRGYILRVTAYKEGYGEGCVT | |
| | | | IHEIENNTDELKFSNCVEEEIYPNNTVTCN | |
| | | | DYTVNQEEYGGAYTSRNRGYNEAPSVPADY | |
| | | | ASVYEEKSYTDGRRENPCEFNRGYRDYTPL | |
| | | | PVGYVTKELEYFPETDKVWIEIGETEGTFI | |
| | | | VDSVELLLMEEMNPNNRSEHDTIKVTPNSE | |
| | | | LQTNHNQYPLADNPNSTLEELNYKEFLRMT | |
| | | | EDSSTEVLDNSTVKDAVGTGISVVGQILGV | |
| | | | VGVPFAGALTSFYQSFLNTIWPSDADPWKA | |
| | | | FMAQVEVLIDKKIEEYAKSKALAELQGLQN | |
| | | | NFEDYVNALNSWKKTPLSLRSKRSQDRIRE | |
| | | | LFSQAESHFRNSMPSFAVSKFEVLFLPTYA | |
| | | | QAANTHLLLLKDAQVFGEEWGYSSEDVAEF | |
| | | | YHRQLKLTQQYTDHCVNWYNVGLNGLRGST | |
| | | | YDAWVKFNRFRREMTLTVLDLIVLFPFYDI | |
| | | | RLYSKGVKTELTRDIFTDPIFSLNTLQEYG | |
| | | | PTFLSIENSIRKPHLFDYLQGIEFHTRLQP | |
| | | | GYFGKDSFNYWSGNYVETRPSIGSSKTITS | |
| | | | PFYGDKSTEPVQKLSFDGQKVYRTIANTDV | |
| | | | AAWPNGKVYLGVTKVDFSQYDDQKNETSTQ | |
| | | | TYDSKRNNGHVSAQDSIDQLPPETTDEPLE | |
| | | | KAYSHQLNYAECFLMQDRRGTIPFFTWTHR | |
| | | | SVDFFNTIDAEKITQLPVVKAYALSSGASI | |
| | | | IEGPGFTGGNLLFLKESSNSIAKFKVTLNS | |
| | | | AALLQRYRVRIRYASTTNLRLFVQNSNNDF | |
| | | | LVIYINKTMNKDDDLTYQTFDLATTNSNMG | |
| | | | FSGDKNELIIGAESFVSNEKIYIDKIEFIP | |
| | | | VQL | |
| 53 | Bacillus | | MNKNNTKLSTRALPSFIDYFNGIYGFATGI | Vip3A |
| | thuingiensis | | KDIMNMIFKTDTGGDLTLDEILKNQQLLND | |
| | | | ISGKLDGVNGSLNDLIAQGNLNTELSKEIL | |
| | | | KIANEQNQVLNDVNNKLDAINTMLRVYLPK | |
| | | | ITSMLSDVMKQNYALSLQIEYLSKQLQEIS | |
| | | | DKLDIINVNVLINSTLTEITPAYQRIKYVN | |
| | | | EKFEELTFATETSSKVKKDGSPADILDELT | |
| | | | ELTELAKSVTKNDVDGFEFYLNTFHDVMVG | |
| | | | NNLFGRSALKTASELITKENVKTSGSEVGN | |
| | | | VYNFLIVLTALQAKAFLTLTTCRKLLGLAD | |
| | | | IDYTSIMNEHLNKEKEEFRVNILPTLSNTF | |
| | | | SNPNYAKVKGSDEDAKMIVEAKPGHALIGF | |
| | | | EISNDSITVLKVYEAKLKQNYQVDKDSLSE | |
| | | | VIYGDMDKLLCPDQSEQIYYTNNIVFPNEY | |
| | | | VITKIDFTKKMKTLRYEVTANFYDSSTGEI | |
| | | | DLNKKKVESSEAEYRTLSANDDGVYMPLGV | |
| | | | ISETFLTPINGFGLQADENSRLITLTCKSY | |
| | | | LRELLLATDLSNKETKLIVPPSGFISNIVE | |
| | | | NGSIEEDNLEPWKANNKNAYVDHTGGVNGT | |
| | | | KALYVHKDGGISQFIGDKLKPKTEYVIQYT | |
| | | | VKGKPSIHLKDENTGYIHYEDTNNNLEDYQ | |
| | | | TINKRFTTGTDLKGVYLILKSQNGDEAWGD | |
| | | | NFIILEISPSEKLLSPELINTNNWTSTGST | |
| | | | NISGNTLTLYQGGRGILKQNLQLDSFSTYR | |
| | | | VYFSVSGDANVRIRNSREVLFEKRYMSGAK | |
| | | | DVSEMFTTKFEKDNFYIELSQGNNLYGGPI | |
| | | | VHFYDVSIK | |
| 54 | Artificial | Protein | GSTSGSGKPGSGEGSTKG | Linker |
| | | | | 218 |
| 55 | Artificial | DNA | GGCTCAACTTCTGGATCTGGGAAGCCAGGG | Linker |
| | | | AGCGGCGAAGGTTCGACTAAGGGC | 218 |
| 56 | Artificial | Protein | AEAAAKEAAAKEAAAKA | linker |
| | | | | AEAAAK3 |
| 57 | Artificial | DNA | GCGGAGGCCGCTGCAAAGGAAGCTGCTGCG | linker |
| | | | AA | |
| | | | GGAAGCAGCGGCCAAGGCG | AEAAAK3 |
| 58 | Artificial | Protein | ESGSVSSEQLAQFRSLD | linker |
| | | | | ESGSV* |
| 59 | Artificial | DNA | GAAAGCGGCAGCGTGAGCAGCGAACAGCTG | linker |
| | | | GC | |
| | | | GCAGTTTCGCAGCCTGGAT | ESGSV* |
| 60 | Artificial | Protein | GGGGSGGGGSGGGGS | linker |
| | | | | Gly4Ser1 |
| | | | | X3* |
| 61 | Artificial | DNA | Ggtggcggcgggagtggcgggggtggtag | linker |
| | | | tggcggtgggggctcc | Gly4Ser1 |
| | | | | X3* |
| 62 | Artificial | Protein | GGGGGGGG | linker |
| | | | | gly8* |
| 63 | Artificial | DNA | Ggtggcggcgggggcgggggtggt | linker |
| | | | | gly8* |
| 64 | Artificial | Protein | PTPTTPTPTTPT | linker |
| | | | | PTPT* |
| 65 | Artificial | DNA | CCGACTCCGACTACTCCCACACCTACCACG | Linker |
| | | | CCCACC | PTPT* |
| 66 | Artificial | Protein | MKYLLPTAAAGLLLLAAQPA | >PelB |
| | | | | secretion |
| | | | | signal |
| | | | | sequence |
| | | | | amino |
| | | | | acids (for |
| | | | | expression |
| | | | | of |
| | | | | BsNbs in |
| | | | | Ecoli) |
| 67 | Artificial | DNA | atggccCAGGTGCAGCTGCAGGAGTCTGGG | Cry1F |
| | | | GGAGGCTTGGTGCAGCCGGGGGGGTCTCTG | |
| | | | AGACTCTCCTGTGCAGCCTCTGGATTCACC | monospecific |
| | | | TTCAGTAGCGCTGCCATGAGCTGGGTCCGC | nanobody |
| | | | CAGGCTCCAGGAAAGGGGCTCGAGTGGGTC | #5* |
| | | | TCAGTTATTAATAGTGGCGGTGATAGTACA | |
| | | | AGTTATACAGGCTCCGTGAAGGGCCGATTC | |
| | | | ACCATCTCCAGGGACAACGCCAAGGCGACA | |
| | | | CTGTATCTGCAAATGAACAACCTGAAACCT | |
| | | | GAGGACACGGCCGTGTATTATTGTGCAAGA | |
| | | | GGGAGTACCCGCGGCCAGGGGACCCAGGTC | |
| | | | ACCGTCTCCTCAGCGGCCGCATACCCGTAC | |
| | | | GACGTTCCGGACTACGGTTCCCACCACCAT | |
| | | | CACCATCACTAG | |
| 68 | Artificial | Protein | MAQVQLQESGGGLVQPGGSLRLSCAASGFT | Cry1F |
| | | | FSSAAMSWVRQAPGKGLEWVSVINSGGDST | monospecific |
| | | | SYTGSVKGRFTISRDNAKATLYLQMNNLKP | nanobody |
| | | | EDTAVYYCARGSTRGQGTQVTVSSAAAYPY | #5* |
| | | | DVPDYGSHHHHHH | |
| 69 | Artificial | DNA | atggccCAGGTGCAGCTGCAGGAGTCTGGG | cry1F |
| | | | GGAGGCTCGGTGCAGCCTGGGGGGTCTCTA | monospecific |
| | | | AAACTCTCCTGTGCAGCCTCTGGATTCACC | nanobody |
| | | | TTCAGTAACTATGCCATGAGCTGGGTCCGC | #7* |
| | | | CAGGCTCCAGGAAAGGGGCTCGAGTGGGTC | |
| | | | TCAGGTATTAATGCTGGTGGTGATACGACA | |
| | | | AACTATGCAGACTCCGTGAAGGACCGATTC | |
| | | | ACCATCTCCAGAGACAACGCCAAGAACACG | |
| | | | CTGTATCTCCAAATGAACAGCCTGAAACCT | |
| | | | GAGGACACGGCCATATATTACTGTCTAAAG | |
| | | | CTTGAGACCAGTGTGGTTCCTAGTAGTAGT | |
| | | | TACTACCGCAATCGCGGTTCCAGGGGCCAG | |
| | | | GGAACCCAGGTCACCGTCTCCTCAGCGGCC | |
| | | | GCATACCCGTACGACGTTCCGGACTACGGT | |
| | | | TCCCACCACCATCACCATCACTAG | |
| 70 | Artificial | Protein | MAQVQLQESGGGSVQPGGSLKLSCAASGFT | cry1F |
| | | | FSNYAMSWRQAPGKGLEWVSGINAGGDTTN | monospecific |
| | | | YADSVKDRFTISRDNAKNTLYLQMNSLKPE | nanobody |
| | | | DTAIYYCLKLETSVVPSSSYYRNRGSRGQG | #7* |
| | | | TQVTVSSAAAYPYDVPDYGSHHHHHH | |
| 71 | Artificial | DNA | atggccCAGGTGCAGCTGCAGGAGTCTGGG | cry1F |
| | | | GGAGGATTGGTGCAGGCTGGGGACTCTCTG | monospecific |
| | | | AGACTCTCCTGTGCAGCCTCTGAACAATCC | nanobody |
| | | | TTCAATAGCGAAATTATGGGCTGGTTCCGC | #51* |
| | | | CAGGCTCCAGGGAAGGAGCGTGAGTTTGTA | |
| | | | GCAGCTATTACCTATAGTGGTAGTATCACA | |
| | | | AAATATGCAGACTCCGCGAAGGGCCGATTC | |
| | | | ACCATCTCCAGAGACAACGCCAAGAACACG | |
| | | | GTGTATCTGCAAATGAACAGTTTGAACCCT | |
| | | | GAGGACACGGCCCTTTATTACTGTGCCCTT | |
| | | | AACAAAGGGGGACTGTATACTGACTACCGA | |
| | | | TCTTGGGCGACGTATGACTACCGCGGCCAG | |
| | | | GGGACCCAGGTCACCGTCTCCTCAGCGGCC | |
| | | | GCATACCCGTACGACGTTCCGGACTACGGT | |
| | | | TCCCACCACCATCACCATCACTAG | |
| 72 | Artificial | Protein | MAQVQLQESGGGLVQAGDSLRLSCAASEQS | cry1F |
| | | | FNSEIMGWFRQAPGKEREFVAAITYSGSIT | monospecific |
| | | | KYADSAKGRFTISRDNAKNTVYLQMNSLNP | nanobody |
| | | | EDTALYYCALNKGGLYTDYRSWATYDYRGQ | #51* |
| | | | GTQVTVSSAAAYPYDVPDYGSHHHHHH | |
| 73 | Artificial | DNA | atggccCAGGTGCAGCTGCAGGAGTCTGGG | NAAT |
| | | | GGAGGATTGGTGCAGGCTGGGGGCTCTCTG | monospecific |
| | | | AGACTCTCCTGTGCAGCCTCTGGACGCACC | nanobody |
| | | | TTCAGTAGGTATGCCATGGGCTGGTTTCGC | #1* |
| | | | CAGGCTCTAGGGAAGGAGCGTGAGTTCGTA | |
| | | | GCAGGTATTAACTGGAGTGGTAGTATGACA | |
| | | | TACTATGCAGACTCCGTGAAGGGCCGATTC | |
| | | | ACCATCTCCAGAGACAACGCCAAGAACATG | |
| | | | CTGTATCTGCAAATGAACAGTCTGAAATCT | |
| | | | GAGGACACGGCCGTGTATTACTGTGCCGGC | |
| | | | GTGACGGTAGTAGGTGGTGCACCAGCCTTT | |
| | | | GACTACTGGGGCCAGGGGACCCAGGTCACC | |
| | | | GTCTCCTCAGCGGCCGCATACCCGTACGAC | |
| | | | GTTCCGGACTACGGTTCCCACCACCATCAC | |
| | | | CATCACTAG | |
| 74 | Artificial | Protein | MAQVQLQESGGGLVQAGGSLRLSCAASGRT | NAAT |
| | | | FSRYAMGWFRQALGKEREFVAGINWSGSMT | monospecific |
| | | | YYADSVKGRFTISRDNAKNMLYLQMNSLKS | nanobody |
| | | | EDTAVYYCAGVTVVGGAPAFDYWGQGTQVT | #1 |
| | | | VSSAAAYPYDVPDYGSHHHHHH | |
| 75 | Artificial | DNA | atggccCAGGTGCAGCTGCAGGAGTCTGGA | NAAT |
| | | | GGAGGAGTGGTGCAGACTGGGGGCTCCCTG | monospecific |
| | | | ACACTCTCCTGTAAAGCCTCTAGACGCACC | nanobody |
| | | | AGTGGCTTTGCCATGGCCTGGTTCCGCCAG | #2* |
| | | | GCTCCAGGGATGGAACGTGAATTTGTAGCG | |
| | | | GGCATTGGTCGGACTGGGGATAATATCCAC | |
| | | | TATTTAGATTCTGTGAAGGGCCGATTCACC | |
| | | | ATCTCTAGAGATAATACCAAGAACACGCTG | |
| | | | TCTCTGCAAATGAACAGCCTGAGACCTGGG | |
| | | | GACACGGCCGTCTATTACTGTGCAGCAGAC | |
| | | | GTGACAAAGAGTGGATTTATTTATTGGGGC | |
| | | | CAGGGGACCCAGGTCACCGTCTCCTCAGCG | |
| | | | GCCGCATACCCGTACGACGTTCCGGACTAC | |
| | | | GGTTCCCACCACCATCACCATCACTAG | |
| 76 | Artificial | Protein | MAQVQLQESGGGVVQTGGSLTLSCKASRRT | NAAT |
| | | | SGFAMAWFRQAPGMEREFVAGIGRTGDNIH | monospecific |
| | | | YLDSVKGRFTISRDNTKNTLSLQMNSLRPG | nanobody |
| | | | DTAVYYCAADVTKSGFIYWGQGTQVTVSSA | #2 |
| | | | AAYPYDVPDYGSHHHHHH | |
| 77 | Artificial | DNA | atggccCAGGTGCAGCTGCAGGAGTCTGGG | NAAT |
| | | | GGAGGCTTGGTGCAGCCTGGGGGGTCTCTG | monospecific |
| | | | AGACTCGCCTGTGTACTCTCCGGAGGCCCC | nanobody |
| | | | TCGAGTAGTTATGGTGTGGGCTGGTTCCGA | #5* |
| | | | CAGCGATCAGGGACAGAGCGTGAATTTGTA | |
| | | | GCAGCTATCAGTGGGAGTGGTCGTACTATC | |
| | | | CATTATGTAGACGACGTGAAGGGCCGATTC | |
| | | | GCCATCTCCAGAGACAGCGCCAAGAATGCG | |
| | | | GTGGATCTGCAAATGAACAACCTGAAACCT | |
| | | | GAGGACACGGCCGTTTATTACTGTGCGGCA | |
| | | | CTCGCGCTCGTTACTACTCATCCGACGAGC | |
| | | | AATGTGGGTGAATGGGACTACTGGGGCCAG | |
| | | | GGGACCCAGGTCACCGTCTCCTCAGCGGCC | |
| | | | GCATACCCGTACGACGTTCCGGACTACGGT | |
| | | | TCCCACCACCATCACCATCACTAG | |
| 78 | Artificial | Protein | MAQVQLQESGGGLVQPGGSLRLACVLSGGP | NAAT |
| | | | SSSYGVGWFRQRSGTEREFVAAISGSGRTI | monospecific |
| | | | HYVDDVKGRFAISRDSAKNAVDLQMNNLKP | nanobody |
| | | | EDTAVYYCAALALVTTHPTSNVGEWDYWGQ | #5 |
| | | | GTQVTVSSAAAYPYDVPDYGSHHHHHH | |
| 79 | Artificial | DNA | atggccCAGGTGCAGCTGCAGGAGTCTGGA | NAAT |
| | | | GGAGGAGTGGTGCAGACTGGGGGCTCCCTG | monospecific |
| | | | ACACTCTCCTGTAAAGCCTCTAGACGCACC | nanobody |
| | | | AGTGGCTTTGCCATGGCCTGGTTCCGCCAG | #6* |
| | | | GCTCCAGGGATGGAACGTGAATTTGTAGCG | |
| | | | GGCATTGGTCGGACTGGGGATAATATCCAC | |
| | | | TATTTAGATTCTGTGAAGGGCCGATTCACC | |
| | | | ATCTCTAGAGATAATACCAAGAACACGCTG | |
| | | | TCTCTGCAAATGAACAGTOTGAAATCTGAG | |
| | | | GACACGGCCGTGTATTACTGTGCAAAAGTG | |
| | | | GTGGTAGTAGCTGGTTCACCATOTTTCGAC | |
| | | | GCATGGGGCCAGGGGACCCAGGTCACCGTC | |
| | | | TCCTCAGCGGCCGCATACCCGTACGACGTT | |
| | | | CCGGACTACGGTTCCCACCACCATCACCAT | |
| | | | CACTAG | |
| 80 | Artificial | Protein | MAQVQLQESGGGVVQTGGSLTLSCKASRRT | NAAT |
| | | | SGFAMAWFRQAPGMEREFVAGIGRTGDNIH | monospecific |
| | | | YLDSVKGRFTISRDNTKNTLSLQMNSLKSE | nanobody |
| | | | DTAVYYCAKVVVVAGSPSFDAWGQGTQVTV | #6* |
| | | | SSAAAYPYDVPDYGSHHHHHH | |
| 81 | Artificial | DNA | atggccCAGGTGCAGCTGCAGGAGTCTGGG | NAAT |
| | | | GGAGGATTGGTGCAGGCTGGGGGCTCTCTG | monospecific |
| | | | ACGCTCTCCTGCGCATTCTCTGGTCGCACC | nanobody |
| | | | TTCACTCATTATGCCATGGCCTGGTTCCGC | #10* |
| | | | CAGGCTCCAGGGAAGGAGCGTAAGTTCGTA | |
| | | | GCTGGTGTTACACGGAGTGGCCCGAACACA | |
| | | | TATTATGACGACTCCGTGCAGGGCCGATTC | |
| | | | ACCATCTCAAGAGACAACGCCAAGAACACG | |
| | | | GTTTATCTGCACATGAACAGCCTGAAACCT | |
| | | | GAGGACACGGCCGTTTATTACTGTGCTGCA | |
| | | | AATTCGGGGGTAGTATCCGGATATGACTAC | |
| | | | TGGGGCCAGGGGACCCAGGTCACCGTCTCC | |
| | | | TCAGCGGCCGCATACCCGTACGACGTTCCG | |
| | | | GACTACGGTTCCCACCACCATCACCATCAC | |
| | | | TAG | |
| 82 | Artificial | Protein | MAQVQLQESGGGLVQAGGSLTLSCAFSGRT | NAAT |
| | | | FTHYAMAWFRQAPGKERKFVAGVTRSGPNT | monospecific |
| | | | YYDDSVQGRFTISRDNAKNTVYLHMNSLKP | nanobody |
| | | | EDTAVYYCAANSGVVSGYDYWGQGTQVTVS | #10* |
| | | | SAAAYPYDVPDYGSHHHHHH | |
| 83 | Artificial | DNA | atggccCAGGTTCAGTTGCAGGAAAGCGGT | NAAT |
| | | | GGCGGCGTAGTTCAAACTGGCGGTTCGCTG | monospecific |
| | | | ACACTTTCTTGTAAGGCTTCTCGTCGCACT | nanobody |
| | | | TCCGGGTTTGCAATGGCCTGGTTCCGTCAG | #29* |
| | | | GCTCCGGGGATGGAGCGTGAGTTTGTCGCG | |
| | | | GGGATTGGGCGCACGGGGGACAACATCCAT | |
| | | | TACCTTGATTCGGTCAAAGGCCGCTTCACT | |
| | | | ATTTCTCGTGATAACACAAAGAACACTCTG | |
| | | | AGCTTACAAATGAATAATCTTAAACCGGAG | |
| | | | GACACGGCTGTTTATTATTGCTTGCGTACT | |
| | | | ATGGGAGGGACCTGGTCGGAGAAGGGGCAA | |
| | | | GGCACGCAGGTCACGGTTAGCTCAGCGGCC | |
| | | | GCATACCCGTACGACGTTCCGGACTACGGT | |
| | | | TCCCACCACCATCACCATCACTAG | |
| 84 | Artificial | Protein | MAQVQLQESGGGVVQTGGSLTLSCKASRRT | NAAT |
| | | | SGFAMAWFRQAPGMEREFVAGIGRTGDNIH | monospecific |
| | | | YLDSVKGRFTISRDNTKNTLSLQMNNLKPE | nanobody |
| | | | DTAVYYCLRTMGGTWSEKGQGTQVTVSSAA | #29* |
| | | | AYPYDVPDYGSHHHHHH | |
| 85 | Artificial | DNA | atggccCAGGTGCAGCTGCAGGAGTCTGGG | Cadherin |
| | | | GGAGGATTGGTGCAGGCTGGGGCCTCTCTG | monospecific |
| | | | AGACTCTCCTGTGCAGCCTCTAGACGCACC | nanobody |
| | | | GGCAGTAGTCTTACCATGGGCTGGTTCCGC | #2* |
| | | | CAGGCTCCAGGGAAGGAGCGTGAGTTTGTA | |
| | | | GCAGCTATTAGCCGGAGTGGTATTAGAACA | |
| | | | TACTACGCAGACTTTGTGAAGGGCCGGTTC | |
| | | | ACCATCTCCAGAGACAACGCCAAGAACACG | |
| | | | CTCTATCTGCAAATGAACAGCCTGAAACCT | |
| | | | GAGGACACGGCCGTGTATTACTGTGCGGCT | |
| | | | AACGACAAAACATACGGTAGTGGTCTTGAC | |
| | | | GTCTACACAAGGCGACAAAACTATTACTAC | |
| | | | TGGGGCCCGGGGACCCAGGTCACCGTCTCC | |
| | | | TCAGCGGCCGCATACCCGTACGACGTTCCG | |
| | | | GACTACGGTTCCCACCACCATCACCATCAC | |
| | | | TAG | |
| 86 | Artificial | Protein | MAQVQLQESGGGLVQAGASLRLSCAASRRT | Cadherin |
| | | | GSSLTMGWFRQAPGKEREFVAAISRSGIRT | monospecific |
| | | | YYADFVKGRFTISRDNAKNTLYLQMNSLKP | nanobody |
| | | | EDTAVYYCAANDKTYGSGLDVYTRRQNYYY | #2* |
| | | | WGPGTQVTVSSAAAYPYDVPDYGS | |
| | | | HHHHHH | |
| 87 | Artificial | DNA | atggccCAGGTGCAGCTGCAGGAGTCTGGA | Cadherin |
| | | | GGAGGATTGGCGCAGGCTGGGGCCTCTCTG | monospecific |
| | | | AGACTCTCCTGTGCAGCCTCTAGACGCACC | nanobody |
| | | | GGCAGTAGTCTTACCATGGGCTGGTTCCGC | #43* |
| | | | CAGGCTCCAGGGAAGGAGCGTGAGTTTGTA | |
| | | | GCAGCTATTAGCCGGAGTGGTATTAGAACA | |
| | | | TACTACGCAGACTTTGTGAAGGGCCGGTTC | |
| | | | ACCATCTCCAGAGACAACGCCAAGAACACG | |
| | | | CTCTATCTGCAAATGAACAGCCTGAAACCT | |
| | | | GAGGACACGGCCGTGTATTACTGTGCGGCT | |
| | | | AACGACAAAACATACGGTAGTGGTCTTGAC | |
| | | | GTCTACACAAGGCGACAAGACTATTACTAC | |
| | | | TGGGGCCCGGGGACCCAGGTCACCGTCTCC | |
| | | | TCAGCGGCCGCATACCCGTACGACGTTCCG | |
| | | | GACTACGGTTCCCACCACCATCACCATCAC | |
| | | | TAG | |
| 88 | Artificial | Protein | MAQVQLQESGGGLAQAGASLRLSCAASRRT | Cadherin |
| | | | GSSLTMGWFRQAPGKEREFVAAISRSGIRT | monospecific |
| | | | YYADFVKGRFTISRDNAKNTLYLQMNSLKP | nanobody |
| | | | EDTAVYYCAANDKTYGSGLDVYTRRQDYYY | #43* |
| | | | WGPGTQVTVSSAAAYPYDVPDYGSHHHHHH | |
| 89 | Artificial | DNA | atggccCAGGTGCAGCTGCAGGAGTCTGGG | Cadherin |
| | | | GGAGGCTCGGTGCAGGCTGGGGGGTCTCTG | monospecific |
| | | | AAACTCTCCTGTGCAGCTTCTGGAACCGAA | nanobody |
| | | | AGCATTTTCATCGTGATGGGCTGGTACCGC | #46* |
| | | | CAGGCTCCAGGGAAAGAGCGCGAGTTGGTC | |
| | | | GCGGCAATGTCATTTGGTGGTGGTACAAAT | |
| | | | GTTACAGACGGCGTGAGGGGCCGATTCACC | |
| | | | ATCTCCAGAGACTTTGACAAGAACACGGTG | |
| | | | GATCTACAAATGAACAACCTAAAAACTGAG | |
| | | | GACACGGCCGTCTATTACTGTAATGCAGTC | |
| | | | CAGTGGGGCCCTCGTGACTACTGGGGCCAG | |
| | | | GGGACCCAGGTCACCGTCTCCTCAGCGGCC | |
| | | | GCATACCCGTACGACGTTCCGGACTACGGT | |
| | | | TCCCACCACCATCACCATCACTAG | |
| 90 | Artificial | Protein | MAQVQLQESGGGSVQAGGSLKLSCAASGTE | Cadherin |
| | | | SIFIVMGWYRQAPGKERELVAAMSFGGGTN | monospecific |
| | | | VTDGVRGRFTISRDFDKNTVDLQMNNLKTE | nanobody |
| | | | DTAVYYCNAVQWGPRDYWGQGTQVTVSSAA | #46* |
| | | | AYPYDVPDYGSHHHHHH | |
| 91 | Artificial | DNA | atggccCAGGTGCAGCTGCAGGAGTCTGGA | Cadherin |
| | | | GGAGGATTGGTGCAGGCTGGGGCCTCTCTG | monospecific |
| | | | AGACTCTCCTGTGCAGCCTCTAGACGCACC | nanobody |
| | | | GGCAGTAGTCTTACCATGGGCTGGTTCCGC | #48* |
| | | | CAGGCTCCAGGGAGGGAGCGTGAGTTTGTA | |
| | | | GCAGCTATTAGCCGGAGTGGTATTAGAACA | |
| | | | TACTACGCAGACTTTGTGAAGGGCCGGTTC | |
| | | | ACCATCTCCAGAGACAACGCCAAGAACACG | |
| | | | CTCTATOTGCAAATGAACAGCCTGAAACCT | |
| | | | GAGGACACGGCCGTGTATTACTGTGCGGCT | |
| | | | AACGACAAAACATACGGTAGTGGTCTTGAC | |
| | | | GTCTACACAAGGCGACAAGACTATTACTAC | |
| | | | TGGGGCCCGGGGACCCAGGTCACCGTCTCC | |
| | | | TCAGCGGCCGCATACCCGTACGACGTTCCG | |
| | | | GACTACGGTTCCCACCACCATCACCATCAC | |
| | | | TAG | |
| 92 | Artificial | Protein | MAQVQLQESGGGLVQAGASLRLSCAASRRT | Cadherin |
| | | | GSSLTMGWFRQAPGREREFVAAISRSGIRT | monospecific |
| | | | YYADFVKGRFTISRDNAKNTLYLQMNSLKP | nanobody |
| | | | EDTAVYYCAANDKTYGSGLDVYTRRQDYYY | #48* |
| | | | WGPGTQVTVSSAAAYPYDVPDYGSHHHHHH | |
| 93 | Artificial | DNA | atggccCAGGTGCAGCTGCAGGAGTCTGGG | Cadherin |
| | | | GGAGGATTGGTGCAGGCTGGGGCCTCTCTG | monospecific |
| | | | AGACTCTCCTGTGCAGCCTCTAGACGCACC | nanobody |
| | | | GGCAGTAGTCTTACCATGGGCTGGTTCCGC | #50* |
| | | | CAGGCTCCAGGGAAGGAGCGTGAGTTTGTA | |
| | | | GCAGCTATTAGCCGGAGTGGTATTAGAACA | |
| | | | TACTACGCAGACTTTGTGAAGGGCCGGTTC | |
| | | | ACCATCTCCAGAGATAACGGCGAGAATACG | |
| | | | CTGTATCTGCAAATCAACAGCCTGAAACCT | |
| | | | GAGGACACGGCCGTTTATTACTGTGCGGCA | |
| | | | TCCAGCAACCCGGGATACTATCGTACAGCT | |
| | | | CCCAACCAGTATAACTTCTGGGGCCCGGGG | |
| | | | ACCCAGGTCACCGTCTCCTCTGCGGCCGCA | |
| | | | TACCCGTACGACGTTCCGGACTACGGTTCC | |
| | | | CACCACCATCACCATCACTAG | |
| 94 | Artificial | Protein | MAQVQLQESGGGLVQAGASLRLSCAASRRT | Cadherin |
| | | | GSSLTMGWFRQAPGKEREFVAAISRSGIRT | monospecific |
| | | | YYADFVKGRFTISRDNGENTLYLQINSLKP | nanobody |
| | | | EDTAVYYCAASSNPGYYRTAPNQYNFWGPG | #50* |
| | | | TQVTVSSAAAYPYDVPDYGSHHHHHH | |
| 95 | Artificial | DNA | atggccCAGGTGCAGCTGCAGGAGTCTGGG | cry1FNb5 |
| | | | GGAGGATTGGTGCAGGCTGGGGACTCTCTG | 1-PTPT- |
| | | | AGACTCTCCTGTGCAGCCTCTGAACAATCC | NAAT29 |
| | | | TTCAATAGCGAAATTATGGGCTGGTTCCGC | (aka #22)* |
| | | | CAGGCTCCAGGGAAGGAGCGTGAGTTTGTA | |
| | | | GCAGCTATTACCTATAGTGGTAGTATCACA | |
| | | | AAATATGCAGACTCCGCGAAGGGCCGATTC | |
| | | | ACCATCTCCAGAGACAACGCCAAGAACACG | |
| | | | GTGTATCTGCAAATGAACAGTTTGAACCCT | |
| | | | GAGGACACGGCCCTTTATTACTGTGCCCTT | |
| | | | AACAAAGGGGGACTGTATACTGACTACCGA | |
| | | | TCTTGGGCGACGTATGACTACCGCGGCCAG | |
| | | | GGGACCCAGGTCACCGTCTCCTCACCGACT | |
| | | | CCGACTACTCCCACACCTACCACGCCCACC | |
| | | | CAGGTGCAGCTGCAGGAGTCTGGAGGAGGA | |
| | | | GTGGTGCAGACTGGGGGCTCCCTGACACTC | |
| | | | TCCTGTAAAGCCTCTAGACGCACCAGTGGC | |
| | | | TTTGCCATGGCCTGGTTCCGCCAGGCTCCA | |
| | | | GGGATGGAACGTGAATTTGTAGCGGGCATT | |
| | | | GGTCGGACTGGGGATAATATCCACTATTTA | |
| | | | GATTCTGTGAAGGGCCGATTCACCATCTCT | |
| | | | AGAGATAATACCAAGAACACGCTGTCTCTG | |
| | | | CAAATGAACAACCTGAAACCTGAGGACACG | |
| | | | GCCGTGTATTACTGCCTACGTACGATGGGT | |
| | | | GGTACCTGGTCTGAGAAGGGCCAGGGGACC | |
| | | | CAGGTCACCGTCTCCTCAGCGGCCGCATAC | |
| | | | CCGTACGACGTTCCGGACTACGGTTCCCAC | |
| | | | CACCATCACCATCACTAG | |
| 96 | Artificial | Protein | MAQVQLQESGGGLVQAGDSLRLSCAASEQS | cry1FNb5 |
| | | | FNSEIMGWFRQAPGKEREFVAAITYSGSIT | 1-PTPT- |
| | | | KYADSAKGRFTISRDNAKNTVYLQMNSLNP | NAAT29 |
| | | | EDTALYYCALNKGGLYTDYRSWATYDYRGQ | (aka #22)* |
| | | | GTQVTVSSPTPTTPTPTTPTQVQLQESGGG | |
| | | | VVQTGGSLTLSCKASRRTSGFAMAWFRQAP | |
| | | | GMEREFVAGIGRTGDNIHYLDSVKGRFTIS | |
| | | | RDNTKNTLSLQMNNLKPEDTAVYYCLRTMG | |
| | | | GTWSEKGQGTQVTVSSAAAYPYDVPDYGS | |
| | | | HHHHHH | |
| 97 | Artificial | DNA | ATGGCCCAGGTGCAGCTGCAGGAGTCTGGG | cry1FNb5 |
| | | | GGAGGATTGGTGCAGGCTGGGGACTCTCTG | 1-PTPT- |
| | | | AGACTCTCCTGTGCAGCCTCTGAACAATCC | Cad48 |
| | | | TTCAATAGCGAAATTATGGGCTGGTTCCGC | (aka #43)* |
| | | | CAGGCTCCAGGGAAGGAGCGTGAGTTTGTA | |
| | | | GCAGCTATTACCTATAGTGGTAGTATCACA | |
| | | | AAATATGCAGACTCCGCGAAGGGCCGATTC | |
| | | | ACCATCTCCAGAGACAACGCCAAGAACACG | |
| | | | GTGTATCTGCAAATGAACAGTTTGAACCCT | |
| | | | GAGGACACGGCCCTTTATTACTGTGCCCTT | |
| | | | AACAAAGGGGGACTGTATACTGACTACCGA | |
| | | | TCTTGGGCGACGTATGACTACCGCGGCCAG | |
| | | | GGGACCCAGGTCACCGTCTCCTCACCGACA | |
| | | | CCGACCACCCCCACGCCCACTACTCCCACC | |
| | | | CAAGTCCAGTTACAGGAGTCAGGAGGTGGT | |
| | | | CTGGTCCAAGCAGGTGCCAGTTTGCGTCTT | |
| | | | AGTTGCGCGGCCTCACGCCGTACTGGTAGT | |
| | | | AGTCTGACTATGGGGTGGTTTCGCCAAGCC | |
| | | | CCAGGTCGTGAACGTGAATTTGTCGCAGCG | |
| | | | ATTAGCCGTAGCGGTATCCGTACCTATTAT | |
| | | | GCGGACTTTGTCAAGGGCCGTTTCACTATC | |
| | | | TCCCGCGACAATGCGAAAAACACTTTGTAC | |
| | | | CTTCAAATGAACTCATTGAAACCAGAGGAT | |
| | | | ACCGCGGTGTACTATTGTGCGGCGAACGAC | |
| | | | AAGACGTATGGGTCGGGGCTGGATGTATAT | |
| | | | ACGCGTCGTCAGGATTATTACTACTGGGGT | |
| | | | CCCGGTACCCAGGTAACAGTGTCATCAGCG | |
| | | | GCCGCATACCCGTACGACGTTCCGGACTAC | |
| | | | GGTTCCCACCACCATCACCATCACTAG | |
| 98 | Artificial | Protein | MAQVQLQESGGGLVQAGDSLRLSCAASEQS | cry1FNb5 |
| | | | FNSEIMGWFRQAPGKEREFVAAITYSGSIT | 1-PTPT- |
| | | | KYADSAKGRFTISRDNAKNTVYLQMNSLNP | Cad48 |
| | | | EDTALYYCALNKGGLYTDYRSWATYDYRGQ | (aka #43)* |
| | | | GTQVTVSSPTPTTPTPTTPTQVQLQESGGG | |
| | | | LVQAGASLRLSCAASRRTGSSLTMGWFRQA | |
| | | | PGREREFVAAISRSGIRTYYADFVKGRFTI | |
| | | | SRDNAKNTLYLQMNSLKPEDTAVYYCAAND | |
| | | | KTYGSGLDVYTRRQDYYYWGPGTQVTVSSA | |
| | | | AAYPYDVPDYGSHHHHHH | |
| 99 | Artificial | DNA | ATGGCCCAGGTGCAGCTGCAGGAGTCTGGG | cry1FNb5 |
| | | | GGAGGATTGGTGCAGGCTGGGGACTCTCTG | 1-PTPT- |
| | | | AGACTCTCCTGTGCAGCCTCTGAACAATCC | Cad2 (aka |
| | | | TTCAATAGCGAAATTATGGGCTGGTTCCGC | #48)* |
| | | | CAGGCTCCAGGGAAGGAGCGTGAGTTTGTA | |
| | | | GCAGCTATTACCTATAGTGGTAGTATCACA | |
| | | | AAATATGCAGACTCCGCGAAGGGCCGATTC | |
| | | | ACCATCTCCAGAGACAACGCCAAGAACACG | |
| | | | GTGTATCTGCAAATGAACAGTTTGAACCCT | |
| | | | GAGGACACGGCCCTTTATTACTGTGCCCTT | |
| | | | AACAAAGGGGGACTGTATACTGACTACCGA | |
| | | | TCTTGGGCGACGTATGACTACCGCGGCCAG | |
| | | | GGGACCCAGGTCACCGTCTCCTCACCGACG | |
| | | | CCGACAACCCCTACACCGACTACTCCGACT | |
| | | | CAAGTTCAATTACAGGAATCAGGCGGAGGC | |
| | | | TTGGTACAGGCTGGTGCATCGTTACGTCTT | |
| | | | TCTTGCGCAGCTTCCCGTCGCACGGGCTCT | |
| | | | TCTTTAACGATGGGCTGGTTTCGTCAGGCG | |
| | | | CCGGGCAAAGAGCGTGAATTCGTGGCGGCC | |
| | | | ATCTCGCGTAGCGGGATTCGTACTTATTAT | |
| | | | GCCGACTTTGTCAAAGGCCGCTTCACTATT | |
| | | | AGTCGTGATAATGCAAAGAACACCCTTTAC | |
| | | | CTGCAAATGAATAGCTTGAAGCCGGAAGAC | |
| | | | ACTGCCGTTTACTACTGCGCGGCAAACGAC | |
| | | | AAGACATATGGGTCTGGCCTTGACGTTTAT | |
| | | | ACCCGTCGTCAAAATTATTATTATTGGGGG | |
| | | | CCTGGTACTCAAGTTACCGTGTCGTCAGCG | |
| | | | GCCGCATACCCGTACGACGTTCCGGACTAC | |
| | | | GGTTCCCACCACCATCACCATCACTAG | |
| 100 | Artificial | Protein | MAQVQLQESGGGLVQAGDSLRLSCAASEQS | cry1FNb5 |
| | | | FNSEIMGWFRQAPGKEREFVAAITYSGSIT | 1-PTPT- |
| | | | KYADSAKGRFTISRDNAKNTVYLQMNSLNP | Cad2 (aka |
| | | | EDTALYYCALNKGGLYTDYRSWATYDYRGQ | #48)* |
| | | | GTQVTVSSPTPTTPTPTTPTQVQLQESGGG | |
| | | | LVQAGASLRLSCAASRRTGSSLTMGWFRQA | |
| | | | PGKEREFVAAISRSGIRTYYADFVKGRFTI | |
| | | | SRDNAKNTLYLQMNSLKPEDTAVYYCAAND | |
| | | | KTYGSGLDVYTRRQNYYYWGPGTQVTVSSA | |
| | | | AAYPYDVPDYGSHHHHHH | |
| 101 | Artificial | DNA | ATGGCCCAGGTGCAGCTGCAGGAGTCTGGG | cry1FNb5 |
| | | | GGAGGATTGGTGCAGGCTGGGGACTCTCTG | 1-PTPT- |
| | | | AGACTCTCCTGTGCAGCCTCTGAACAATCC | Cad50 |
| | | | TTCAATAGCGAAATTATGGGCTGGTTCCGC | (aka #49)* |
| | | | CAGGCTCCAGGGAAGGAGCGTGAGTTTGTA | |
| | | | GCAGCTATTACCTATAGTGGTAGTATCACA | |
| | | | AAATATGCAGACTCCGCGAAGGGCCGATTC | |
| | | | ACCATCTCCAGAGACAACGCCAAGAACACG | |
| | | | GTGTATCTGCAAATGAACAGTTTGAACCCT | |
| | | | GAGGACACGGCCCTTTATTACTGTGCCCTT | |
| | | | AACAAAGGGGGACTGTATACTGACTACCGA | |
| | | | TCTTGGGCGACGTATGACTACCGCGGCCAG | |
| | | | GGGACCCAGGTCACCGTCTCCTCACCGACC | |
| | | | CCCACAACGCCAACGCCGACTACTCCGACA | |
| | | | CAAGTACAATTACAAGAATCAGGAGGAGGT | |
| | | | TTAGTTCAAGCTGGCGCTTCTTTACGTTTA | |
| | | | AGTTGTGCGGCCTCCCGTCGTACCGGCTCC | |
| | | | TCATTGACAATGGGGTGGTTTCGTCAGGCT | |
| | | | CCTGGGAAGGAGCGTGAATTTGTCGCCGCA | |
| | | | ATTAGCCGTTCTGGCATTCGTACCTACTAC | |
| | | | GCAGATTTTGTCAAGGGGCGTTTCACCATT | |
| | | | TCACGCGATAACGGGGAGAATACGCTTTAC | |
| | | | TTGCAGATCAATTCTCTGAAGCCTGAAGAT | |
| | | | ACCGCGGTGTACTATTGCGCCGCCTCTTCT | |
| | | | AATCCGGGTTATTATCGCACAGCACCAAAC | |
| | | | CAATATAACTTCTGGGGTCCTGGGACACAA | |
| | | | GTGACGGTTTCTTCTGCGGCCGCATACCCG | |
| | | | TACGACGTTCCGGACTACGGTTCCCACCAC | |
| | | | CATCACCATCACTAG | |
| 102 | Artificial | Protein | MAQVQLQESGGGLVQAGDSLRLSCAASEQS | cry1FNb5 |
| | | | FNSEIMGWFRQAPGKEREFVAAITYSGSIT | 1-PTPT- |
| | | | KYADSAKGRFTISRDNAKNTVYLQMNSLNP | Cad50 |
| | | | EDTALYYCALNKGGLYTDYRSWATYDYRGQ | (aka #49)* |
| | | | GTQVTVSSPTPTTPTPTTPTQVQLQESGGG | cry1FNb5 |
| | | | LVQAGASLRLSCAASRRTGSSLTMGWFRQA | |
| | | | PGKEREFVAAISRSGIRTYYADFVKGRFTI | |
| | | | SRDNGENTLYLQINSLKPEDTAVYYCAASS | |
| | | | NPGYYRTAPNQYNFWGPGTQVTVSSAAAYP | |
| | | | YDVPDYGSHHHHHH | |
| 103 | Artificial | DNA | ATGGCCCAGGTGCAGCTGCAGGAGTCTGGG | 1-gly8- |
| | | | GGAGGATTGGTGCAGGCTGGGGACTCTCTG | Cad43 |
| | | | AGACTCTCCTGTGCAGCCTCTGAACAATCC | (aka #50)* |
| | | | TTCAATAGCGAAATTATGGGCTGGTTCCGC | |
| | | | CAGGCTCCAGGGAAGGAGCGTGAGTTTGTA | |
| | | | GCAGCTATTACCTATAGTGGTAGTATCACA | |
| | | | AAATATGCAGACTCCGCGAAGGGCCGATTC | |
| | | | ACCATCTCCAGAGACAACGCCAAGAACACG | |
| | | | GTGTATCTGCAAATGAACAGTTTGAACCCT | |
| | | | GAGGACACGGCCCTTTATTACTGTGCCCTT | |
| | | | AACAAAGGGGGACTGTATACTGACTACCGA | |
| | | | TCTTGGGCGACGTATGACTACCGCGGCCAG | |
| | | | GGGACCCAGGTCACCGTCTCCTCAGGTGGC | |
| | | | GGCGGAGGTGGAGGTGGTCAGGTGCAGCTT | |
| | | | CAAGAATCAGGGGGGGGATTGGCACAAGCC | |
| | | | GGGGCTAGCTTACGCCTGTCCTGCGCGGCG | |
| | | | AGCCGCCGTACAGGCTCAAGTCTTACGATG | |
| | | | GGCTGGTTTCGTCAGGCACCTGGGAAAGAG | |
| | | | CGTGAGTTTGTTGCCGCCATCTCGCGCTCG | |
| | | | GGTATCCGTACCTACTATGCAGATTTCGTT | |
| | | | AAAGGACGCTTCACGATTTCTCGCGATAAT | |
| | | | GCTAAAAACACGCTGTATCTGCAGATGAAT | |
| | | | TCCTTAAAGCCGGAAGATACAGCGGTGTAC | |
| | | | TACTGTGCGGCAAATGATAAGACCTATGGT | |
| | | | TCTGGTCTGGATGTTTATACACGTCGTCAA | |
| | | | GATTATTACTACTGGGGGCCCGGCACGCAG | |
| | | | GTTACTGTGTCGTCAGCGGCCGCATACCCG | |
| | | | TACGACGTTCCGGACTACGGTTCCCACCAC | |
| | | | CATCACCATCACTAG | |
| 104 | Artificial | Protein | MAQVQLQESGGGLVQAGDSLRLSCAASEQS | cry1FNb5 |
| | | | FNSEIMGWFRQAPGKEREFVAAITYSGSIT | 1-gly8- |
| | | | KYADSAKGRFTISRDNAKNTVYLQMNSLNP | Cad43 |
| | | | EDTALYYCALNKGGLYTDYRSWATYDYRGQ | (aka #50)* |
| | | | GTQVTVSSGGGGGGGGQVQLQESGGGLAQA | |
| | | | GASLRLSCAASRRTGSSLTMGWFRQAPGKE | |
| | | | REFVAAISRSGIRTYYADFVKGRFTISRDN | |
| | | | AKNTLYLQMNSLKPEDTAVYYCAANDKTYG | |
| | | | SGLDVYTRRQDYYYWGPGTQVTVSSAAAYP | |
| | | | YDVPDYGSHHHHHH | |
| 105 | Artificial | DNA | ATGGCCCAGGTGCAGCTGCAGGAGTCTGGG | cry1FNb5 |
| | | | GGAGGATTGGTGCAGGCTGGGGACTCTCTG | 1-gly8- |
| | | | AGACTCTCCTGTGCAGCCTCTGAACAATCC | Cad46 |
| | | | TTCAATAGCGAAATTATGGGCTGGTTCCGC | (aka #53)* |
| | | | CAGGCTCCAGGGAAGGAGCGTGAGTTTGTA | |
| | | | GCAGCTATTACCTATAGTGGTAGTATCACA | |
| | | | AAATATGCAGACTCCGCGAAGGGCCGATTC | |
| | | | ACCATCTCCAGAGACAACGCCAAGAACACG | |
| | | | GTGTATCTGCAAATGAACAGTTTGAACCCT | |
| | | | GAGGACACGGCCCTTTATTACTGTGCCCTT | |
| | | | AACAAAGGGGGACTGTATACTGACTACCGA | |
| | | | TCTTGGGCGACGTATGACTACCGCGGCCAG | |
| | | | GGGACCCAGGTCACCGTCTCCTCAGGTGGT | |
| | | | GGCGGCGGAGGAGGCGGACAGGTGCAGCTT | |
| | | | CAAGAGTCAGGAGGCGGTTCTGTACAAGCC | |
| | | | GGGGGATCTCTGAAATTAAGTTGCGCAGCC | |
| | | | TCTGGGACAGAGTCGATTTTTATTGTTATG | |
| | | | GGCTGGTATCGTCAGGCACCTGGCAAGGAA | |
| | | | CGTGAGCTGGTCGCCGCCATGAGTTTTGGG | |
| | | | GGGGGGACGAATGTGACTGACGGAGTTCGT | |
| | | | GGTCGCTTTACCATTTCCCGTGATTTTGAC | |
| | | | AAGAACACGGTCGACCTGCAAATGAACAAT | |
| | | | CTTAAAACCGAGGACACCGCAGTTTACTAT | |
| | | | TGTAATGCTGTGCAATGGGGCCCGCGCGAC | |
| | | | TACTGGGGACAGGGCACTCAGGTCACAGTA | |
| | | | TCCTCAGCGGCCGCATACCCGTACGACGTT | |
| | | | CCGGACTACGGTTCCCACCACCATCACCAT | |
| | | | CACTAG | |
| 106 | | Protein | MAQVQLQESGGGLVQAGDSLRLSCAASEQS | cry1FNb5 |
| | | | FNSEIMGWFRQAPGKEREFVAAITYSGSIT | 1-gly8- |
| | | | KYADSAKGRFTISRDNAKNTVYLQMNSLNP | Cad46 |
| | | | EDTALYYCALNKGGLYTDYRSWATYDYRGQ | (aka #53)* |
| | | | GTQVTVSSGGGGGGGGQVQLQESGGGSVQA | |
| | | | GGSLKLSCAASGTESIFIVMGWYRQAPGKE | |
| | | | RELVAAMSFGGGTNVTDGVRGRFTISRDFD | |
| | | | KNTVDLQMNNLKTEDTAVYYCNAVQWGPRD | |
| | | | YWGQGTQVTVSSAAAYPYDVPDYGS | |
| | | | HHHHHH | |
| 107 | Artificial | DNA | ATGGCCCAGGTGCAGCTGCAGGAGTCTGGG | cry1FNb7 |
| | | | GGAGGCTCGGTGCAGCCTGGGGGGTCTCTA | -PTPT- |
| | | | AAACTCTCCTGTGCAGCCTCTGGATTCACC | NAAT1 |
| | | | TTCAGTAACTATGCCATGAGCTGGGTCCGC | (aka #62)* |
| | | | CAGGCTCCAGGAAAGGGGCTCGAGTGGGTC | |
| | | | TCAGGTATTAATGCTGGTGGTGATACGACA | |
| | | | AACTATGCAGACTCCGTGAAGGACCGATTC | |
| | | | ACCATCTCCAGAGACAACGCCAAGAACACG | |
| | | | CTGTATCTCCAAATGAACAGCCTGAAACCT | |
| | | | GAGGACACGGCCATATATTACTGTCTAAAG | |
| | | | CTTGAGACCAGTGTGGTTCCTAGTAGTAGT | |
| | | | TACTACCGCAATCGCGGTTCCAGGGGCCAG | |
| | | | GGAACCCAGGTCACCGTCTCCTCACCGACG | |
| | | | CCTACTACCCCGACTCCGACCACTCCTACT | |
| | | | CAGGTGCAGCTTCAGGAGTCAGGTGGAGGT | |
| | | | CTTGTTCAGGCTGGTGGATCTCTTCGCTTG | |
| | | | TCTTGCGCTGCGTCGGGTCGTACATTCTCG | |
| | | | CGCTACGCGATGGGCTGGTTCCGTCAGGCT | |
| | | | TTGGGTAAAGAGCGCGAGTTTGTGGCAGGG | |
| | | | ATTAACTGGTCCGGGTCTATGACGTACTAC | |
| | | | GCAGATTCTGTTAAAGGTCGCTTCACTATT | |
| | | | TCTCGTGATAACGCGAAAAACATGTTGTAT | |
| | | | CTGCAAATGAATTCTCTTAAATCGGAGGAT | |
| | | | ACCGCAGTTTACTATTGCGCAGGAGTCACC | |
| | | | GTCGTCGGAGGTGCGCCAGCTTTTGACTAT | |
| | | | TGGGGTCAAGGCACTCAAGTAACCGTCTCA | |
| | | | TCAGCGGCCGCATACCCGTACGACGTTCCG | |
| | | | GACTACGGTTCCCACCACCATCACCATCAC | |
| | | | TAG | |
| 108 | Artificial | Protein | MAQVQLQESGGGSVQPGGSLKLSCAASGFT | cry1FNb7 |
| | | | FSNYAMSWVRQAPGKGLEWVSGINAGGDTT | -PTPT- |
| | | | NYADSVKDRFTISRDNAKNTLYLQMNSLKP | NAAT1 |
| | | | EDTAIYYCLKLETSVVPSSSYYRNRGSRGQ | (aka #62)* |
| | | | GTQVTVSSPTPTTPTPTTPTQVQLQESGGG | |
| | | | LVQAGGSLRLSCAASGRTFSRYAMGWFRQA | |
| | | | LGKEREFVAGINWSGSMTYYADSVKGRFTI | |
| | | | SRDNAKNMLYLQMNSLKSEDTAVYYCAGVT | |
| | | | VVGGAPAFDYWGQGTQVTVSSAAAYPYDVP | |
| | | | DYGSHHHHHH | |
| 109 | Artificial | DNA | ATGGCCCAGGTGCAGCTGCAGGAGTCTGGG | cry1FNb7 |
| | | | GGAGGCTCGGTGCAGCCTGGGGGGTCTCTA | -PTPT- |
| | | | AAACTCTCCTGTGCAGCCTCTGGATTCACC | NAAT2 |
| | | | TTCAGTAACTATGCCATGAGCTGGGTCCGC | (aka #64) |
| | | | CAGGCTCCAGGAAAGGGGCTCGAGTGGGTC | * |
| | | | TCAGGTATTAATGCTGGTGGTGATACGACA | |
| | | | AACTATGCAGACTCCGTGAAGGACCGATTC | |
| | | | ACCATCTCCAGAGACAACGCCAAGAACACG | |
| | | | CTGTATCTCCAAATGAACAGCCTGAAACCT | |
| | | | GAGGACACGGCCATATATTACTGTCTAAAG | |
| | | | CTTGAGACCAGTGTGGTTCCTAGTAGTAGT | |
| | | | TACTACCGCAATCGCGGTTCCAGGGGCCAG | |
| | | | GGAACCCAGGTCACCGTCTCCTCACCGACC | |
| | | | CCAACCACTCCGACGCCCACAACGCCTACT | |
| | | | CAGGTACAATTACAGGAATCAGGGGGTGGT | |
| | | | GTAGTTCAAACGGGGGGCTCATTGACTCTT | |
| | | | TCTTGCAAAGCGTCTCGCCGTACCTCCGGT | |
| | | | TTTGCGATGGCCTGGTTCCGCCAAGCACCA | |
| | | | GGGATGGAACGTGAATTTGTTGCCGGTATT | |
| | | | GGTCGCACTGGTGATAACATCCACTACCTT | |
| | | | GACTCGGTGAAGGGTCGTTTCACCATTTCC | |
| | | | CGCGACAACACCAAAAATACCTTATCCTTG | |
| | | | CAAATGAATTCTCTTCGTCCAGGTGATACC | |
| | | | GCCGTTTATTACTGTGCTGCTGATGTCACT | |
| | | | AAGTCGGGCTTCATCTACTGGGGTCAGGGC | |
| | | | ACACAGGTCACCGTTTCATCAGCGGCCGCA | |
| | | | TACCCGTACGACGTTCCGGACTACGGTTCC | |
| | | | CACCACCATCACCATCACTAG | |
| 110 | Artificial | Protein | MAQVQLQESGGGSVQPGGSLKLSCAASGFT | cry1FNb7 |
| | | | FSNYAMSWVRQAPGKGLEWVSGINAGGDTT | -PTPT- |
| | | | NYADSVKDRFTISRDNAKNTLYLQMNSLKP | NAAT2 |
| | | | EDTAIYYCLKLETSVVPSSSYYRNRGSRGQ | (aka #64)* |
| | | | GTQVTVSSPTPTTPTPTTPTQVQLQESGGG | |
| | | | VVQTGGSLTLSCKASRRTSGFAMAWFRQAP | |
| | | | GMEREFVAGIGRTGDNIHYLDSVKGRFTIS | |
| | | | RDNTKNTLSLQMNSLRPGDTAVYYCAADVT | |
| | | | KSGFIYWGQGTQVTVSSAAAYPYDVPDYGS | |
| | | | HHHHHH | |
| 111 | Artificial | DNA | ATGGCCCAGGTGCAGCTGCAGGAGTCTGGG | cry1FNb7 |
| | | | GGAGGCTCGGTGCAGCCTGGGGGGTCTCTA | -PTPT- |
| | | | AAACTCTCCTGTGCAGCCTCTGGATTCACC | NAAT10 |
| | | | TTCAGTAACTATGCCATGAGCTGGGTCCGC | (aka #76) |
| | | | CAGGCTCCAGGAAAGGGGCTCGAGTGGGTC | * |
| | | | TCAGGTATTAATGCTGGTGGTGATACGACA | |
| | | | AACTATGCAGACTCCGTGAAGGACCGATTC | |
| | | | ACCATCTCCAGAGACAACGCCAAGAACACG | |
| | | | CTGTATCTCCAAATGAACAGCCTGAAACCT | |
| | | | GAGGACACGGCCATATATTACTGTCTAAAG | |
| | | | CTTGAGACCAGTGTGGTTCCTAGTAGTAGT | |
| | | | TACTACCGCAATCGCGGTTCCAGGGGCCAG | |
| | | | GGAACCCAGGTCACCGTCTCCTCACCGACA | |
| | | | CCCACGACTCCCACTCCCACCACTCCTACC | |
| | | | CAAGTGCAGTTACAGGAATCAGGTGGTGGA | |
| | | | CTGGTCCAAGCAGGCGGGTCCCTTACGCTT | |
| | | | TCATGTGCGTTCAGCGGACGTACGTTTACA | |
| | | | CACTATGCTATGGCGTGGTTCCGCCAAGCA | |
| | | | CCTGGGAAAGAACGTAAATTCGTAGCAGGG | |
| | | | GTAACCCGCTCAGGACCGAATACTTATTAT | |
| | | | GATGACTCCGTCCAAGGGCGTTTCACTATC | |
| | | | AGCCGTGATAATGCAAAGAATACTGTGTAC | |
| | | | CTTCACATGAATTCCTTGAAGCCTGAAGAC | |
| | | | ACGGCGGTATATTATTGCGCTGCCAATTCC | |
| | | | GGTGTGGTTTCTGGATATGATTACTGGGGG | |
| | | | CAAGGCACGCAAGTCACGGTGTCGTCAGCG | |
| | | | GCCGCATACCCGTACGACGTTCCGGACTAC | |
| | | | GGTTCCCACCACCATCACCATCACTAG | |
| 112 | | Protein | MAQVQLQESGGGSVQPGGSLKLSCAASGFT | cry1FNb7 |
| | | | FSNYAMSWVRQAPGKGLEWVSGINAGGDTT | -PTPT- |
| | | | NYADSVKDRFTISRDNAKNTLYLQMNSLKP | NAAT10 |
| | | | EDTAIYYCLKLETSVVPSSSYYRNRGSRGQ | (aka #76)* |
| | | | GTQVTVSSPTPTTPTPTTPTQVQLQESGGG | |
| | | | LVQAGGSLTLSCAFSGRTFTHYAMAWFRQA | |
| | | | PGKERKFVAGVTRSGPNTYYDDSVQGRFTI | |
| | | | SRDNAKNTVYLHMNSLKPEDTAVYYCAANS | |
| | | | GVVSGYDYWGQGTQVTVSSAAAYPYDVPDY | |
| | | | GSHHHHHH | |
| 113 | Artificial | DNA | ATGGCCCAGGTGCAGCTGCAGGAGTCTGGG | cry1FNb5 |
| | | | GGAGGATTGGTGCAGGCTGGGGACTCTCTG | 1-gly8- |
| | | | AGACTCTCCTGTGCAGCCTCTGAACAATCC | NAAT5 |
| | | | TTCAATAGCGAAATTATGGGCTGGTTCCGC | amino |
| | | | CAGGCTCCAGGGAAGGAGCGTGAGTTTGTA | acid (aka |
| | | | GCAGCTATTACCTATAGTGGTAGTATCACA | #85)* |
| | | | AAATATGCAGACTCCGCGAAGGGCCGATTC | |
| | | | ACCATCTCCAGAGACAACGCCAAGAACACG | |
| | | | GTGTATCTGCAAATGAACAGTTTGAACCCT | |
| | | | GAGGACACGGCCCTTTATTACTGTGCCCTT | |
| | | | AACAAAGGGGGACTGTATACTGACTACCGA | |
| | | | TCTTGGGCGACGTATGACTACCGCGGCCAG | |
| | | | GGGACCCAGGTCACCGTCTCCTCAGGTGGG | |
| | | | GGAGGAGGTGGTGGTGGTCAGGTACAGTTG | |
| | | | CAGGAGAGTGGAGGCGGTCTTGTGCAACCT | |
| | | | GGCGGGTCGTTGCGCTTAGCTTGTGTATTG | |
| | | | TCGGGCGGTCCCTCGTCGTCCTATGGGGTA | |
| | | | GGGTGGTTTCGTCAGCGCAGTGGCACAGAA | |
| | | | CGCGAATTCGTGGCTGCTATCTCTGGGTCC | |
| | | | GGTCGCACAATCCATTATGTTGACGATGTA | |
| | | | AAAGGACGCTTCGCAATTTCACGCGATAGC | |
| | | | GCTAAGAATGCAGTGGACTTGCAGATGAAC | |
| | | | AATCTGAAACCGGAGGATACAGCAGTTTAT | |
| | | | TACTGTGCTGCTCTTGCACTGGTAACGACG | |
| | | | CATCCGACGTCTAACGTCGGAGAATGGGAC | |
| | | | TACTGGGGTCAAGGCACCCAGGTTACCGTG | |
| | | | AGTTCAGCGGCCGCATACCCGTACGACGTT | |
| | | | CCGGACTACGGTTCCCACCACCATCACCAT | |
| | | | CACTAG | |
| 114 | Artificial | Protein | MAQVQLQESGGGLVQAGDSLRLSCAASEQS | cry1FNb5 |
| | | | FNSEIMGWFRQAPGKEREFVAAITYSGSIT | 1-gly8- |
| | | | KYADSAKGRFTISRDNAKNTVYLQMNSLNP | NAAT5 |
| | | | EDTALYYCALNKGGLYTDYRSWATYDYRGQ | (aka #85)* |
| | | | GTQVTVSSGGGGGGGGQVQLQESGGGLVQP | |
| | | | GGSLRLACVLSGGPSSSYGVGWFRQRSGTE | |
| | | | REFVAAISGSGRTIHYVDDVKGRFAISRDS | |
| | | | AKNAVDLQMNNLKPEDTAVYYCAALALVTT | |
| | | | HPTSNVGEWDYWGQGTQVTVSSAAAYPYDV | |
| | | | PDYGSHHHHHH | |
| 115 | Artificial | DNA | ATGGCCCAGGTGCAGCTGCAGGAGTCTGGG | cry1FNb5 |
| | | | GGAGGATTGGTGCAGGCTGGGGACTCTCTG | 1-gly8- |
| | | | AGACTCTCCTGTGCAGCCTCTGAACAATCC | NAAT6 |
| | | | TTCAATAGCGAAATTATGGGCTGGTTCCGC | (aka #87)* |
| | | | CAGGCTCCAGGGAAGGAGCGTGAGTTTGTA | |
| | | | GCAGCTATTACCTATAGTGGTAGTATCACA | |
| | | | AAATATGCAGACTCCGCGAAGGGCCGATTC | |
| | | | ACCATCTCCAGAGACAACGCCAAGAACACG | |
| | | | GTGTATCTGCAAATGAACAGTTTGAACCCT | |
| | | | GAGGACACGGCCCTTTATTACTGTGCCCTT | |
| | | | AACAAAGGGGGACTGTATACTGACTACCGA | |
| | | | TCTTGGGCGACGTATGACTACCGCGGCCAG | |
| | | | GGGACCCAGGTCACCGTCTCCTCAGGTGGC | |
| | | | GGAGGTGGAGGCGGAGGTCAAGTTCAACTT | |
| | | | CAGGAATCCGGTGGGGGTGTAGTACAAACG | |
| | | | GGAGGGTCTCTTACGCTGTCCTGCAAGGCA | |
| | | | TCTCGCCGCACCTCAGGGTTCGCTATGGCC | |
| | | | TGGTTTCGTCAAGCGCCAGGGATGGAACGC | |
| | | | GAGTTCGTTGCTGGAATCGGACGTACTGGT | |
| | | | GACAACATCCATTATCTTGACAGTGTGAAA | |
| | | | GGACGCTTCACCATCAGCCGCGATAATACA | |
| | | | AAAAACACCTTAAGCCTTCAAATGAATTCT | |
| | | | CTGAAGAGCGAGGACACCGCAGTTTATTAT | |
| | | | TGTGCCAAAGTGGTTGTTGTAGCGGGGTCA | |
| | | | CCTAGTTTCGACGCGTGGGGCCAGGGCACG | |
| | | | CAGGTTACCGTGTCCTCAGCGGCCGCATAC | |
| | | | CCGTACGACGTTCCGGACTACGGTTCCCAC | |
| | | | CACCATCACCATCACTAG | |
| 116 | Artificial | Protein | MAQVQLQESGGGLVQAGDSLRLSCAASEQS | cry1FNb5 |
| | | | FNSEIMGWFRQAPGKEREFVAAITYSGSIT | 1-gly8- |
| | | | KYADSAKGRFTISRDNAKNTVYLQMNSLNP | NAAT6 |
| | | | EDTALYYCALNKGGLYTDYRSWATYDYRGQ | (aka #87)* |
| | | | GTQVTVSSGGGGGGGGQVQLQESGGGVVQT | |
| | | | GGSLTLSCKASRRTSGFAMAWFRQAPGMER | |
| | | | EFVAGIGRTGDNIHYLDSVKGRFTISRDNT | |
| | | | KNTLSLQMNSLKSEDTAVYYCAKVVVVAGS | |
| | | | PSFDAWGQGTQVTVSSAAAPYDVPDYGS | |
| | | | HHHHHH | |
| 117 | Artificial | DNA | ATGGCCCAGGTGCAGCTGCAGGAGTCTGGG | Cad49 |
| | | | GGAGGATTGGTGCAGGCTGGGGCCTCTCTG | |
| | | | AGACTCTCCTGTGCAGCCTCTAGACGCACC | |
| | | | GGCAGTAGTCTTACCATGGGCTGGTTCCGC | |
| | | | CAGGCTCCAGGGAAGGAGCGTGAGTTTGTA | |
| | | | GCAGCTATTAGCCGGAGTGGTATTAGAACA | |
| | | | TACTACGCAGACTTTGTGAAGGGCCGGTTC | |
| | | | ACCATCTCCAGAGACAACGCCAAGAACACG | |
| | | | CTCTATCTGCAAATGAACAGCCTGAAACCT | |
| | | | GGGGACACGGCCGTGTATTACTGTGCGGCT | |
| | | | AACGACAAAACATACGGTAGTGGTCTTGAC | |
| | | | GTCTACACAAGGCGACAAGACTATTACTAC | |
| | | | TGGGGCCCGGGGACCCAGGTCACCGTCTCC | |
| | | | TCAGCGGCCGCATACCCGTACGACGTTCCG | |
| | | | GACTACGGTTCCCACCACCATCACCATCAC | |
| | | | TAG | |
| 118 | Artificial | Protein | MAQVQLQESGGGLVQAGASLRLSCAASRRT | Cad49 |
| | | | GSSLTMGWFRQAPGKEREFVAAISRSGIRT | |
| | | | YYADFVKGRFTISRDNAKNTLYLQMNSLKP | |
| | | | GDTAVYYCAANDKTYGSGLDVYTRRQDYYY | |
| | | | WGPGTQVTVSSAAAYPYDVPDYGSHHHHHH | |
| 119 | Artificial | DNA | ATGGCCCAGGTGCAGCTGCAGGAGTCTGGG | Cad51 |
| | | | GGAGGCTCGGTGCAGGCTGGGGGGTCTCTG | |
| | | | AAACTCTCCTGTGCAGCCTCTGGAACCGAA | |
| | | | AGCATTTTCATCGTGATGGGCTGGTACCGC | |
| | | | CAGGCTCCAGGGAAAGAGCGCGAGTTGGTC | |
| | | | GCGGCAATGACATTTGGTGGTGGTACAAAT | |
| | | | GTGACAGACGGCGTGAGGGGCCGATTCACC | |
| | | | ATCTCCAGAGACCTTGACAAGAACACGGTG | |
| | | | GATCTGCAAATGAACAACCTAAAAACTGAG | |
| | | | GACACGGCCGTCTATTACTGTAATGCAGTC | |
| | | | CGTTGGGGCCCTCGTGACTACTGGGGCCAG | |
| | | | GGGACCCAGGTCACCGTCTCCTCAGCGGCC | |
| | | | GCATACCCGTACGACGTTCCGGACTACGGT | |
| | | | TCCCACCACCATCACCATCACTAG | |
| 120 | Artificial | Protein | MAQVQLQESGGGSVQAGGSLKLSCAASGTE | Cad51 |
| | | | SIFIVMGWYRQAPGKERELVAAMTFGGGTN | |
| | | | VTDGVRGRFTISRDLDKNTVDLQMNNLKTE | |
| | | | DTAVYYCNAVRWGPRDYWGQGTQVTVSSAA | |
| | | | AYPYDVPDYGSHHHHHH | |
| 121 | Artificial | DNA | ATGGCCCAGGTGCAGCTGCAGGAGTCTGGG | Cad38 |
| | | | GGAGGATTGGTGCAGGCTGGGGGCTCTCTG | |
| | | | AGACTCTCCTGTGCAGCCTCTGGACGCACT | |
| | | | TTCAGTAGCGCGCCCATGGCCTGGTTCCGC | |
| | | | CAGGTTCCAGGGAAGGAGCGTGAATTTGTC | |
| | | | GCCGCTATTAGCAGTAATGATGGTAGTACA | |
| | | | AGGTATGGAGACTCCGTGAAGGGCCGATTC | |
| | | | ACCATCTCCAGAGACAACGCCAAGAACGCG | |
| | | | CTGTGGCTGCAAATGAACAGCCTGAAACCT | |
| | | | GAGGACACGGCCGTGTATTACTGTGCGGCC | |
| | | | CGGCGAACATTACGGAGTGGTAGTTACACG | |
| | | | CCCGGCGCAACATATTACTATGACTCATGG | |
| | | | GGCCCGGGGACCCAGGTCACCGTCTCCTCA | |
| | | | GCGGCCGCATACCCGTACGACGTTCCGGAC | |
| | | | TACGGTTCCCACCACCATCACCATCACTAG | |
| 122 | Artificial | Protein | MAQVQLQESGGGLVQAGGSLRLSCAASGRT | Cad38 |
| | | | FSSAPMAWFRQVPGKEREFVAAISSNDGST | |
| | | | RYGDSVKGRFTISRDNAKNALWLQMNSLKP | |
| | | | EDTAVYYCAARRTLRSGSYTPGATYYYDSW | |
| | | | GPGTQVTVSSAAAYPYDVPDYGSHHHHHH | |
| 123 | Artificial | DNA | ATGGCCCAGGTGCAGCTGCAGGAGTCTGGA | Cad47 |
| | | | GGAGGCTTGGTGCAGGCTGGGGGGTCTCTG | |
| | | | AGACTCTCCTGTGTAGCCTCTGGAAGCATC | |
| | | | TTCAGTGGCGATGCCATGGGCTGGTACCGC | |
| | | | CAGGCTCCAGGGAAGCAGCGCGAGTTGGTC | |
| | | | GCAACTATTACTCGTGGTGGGATTACAAAC | |
| | | | TATGCCGACTCCGCGAAGGGCCGATTCACC | |
| | | | ATCTCCAGAGACAATGCCAAGAACACGGTG | |
| | | | TCTCTGCAAATGAACAGCCTGAAACCTGAG | |
| | | | GACACGGCCGTCTATTACTGTCATGCAGAA | |
| | | | GACCCGGGTTGGGGTGTCTACCGGGGGCGT | |
| | | | CGTGGCTACTGGGGCCAGGGGACCCAGGTC | |
| | | | ACCGTCTCCTCAGCGGCCGCATACCCGTAC | |
| | | | GACGTTCCGGACTACGGTTCCCACCACCAT | |
| | | | CACCATCACTAG | |
| 124 | Artificial | Protein | QVQLQESGGGLVQAGGSLRLSCVASGSIFS | Cad47 |
| | | | GDAMGWYRQAPGKQRELVATITRGGITNYA | |
| | | | DSAKGRFTISRDNAKNTVSLQMNSLKPEDT | |
| | | | AVYYCHAEDPGWGVYRGRRGYWGQGTQVTV | |
| | | | SSAAAYPYDVPDYGSHHHHHH | |
| 125 | Artificial | DNA | ATGGCCCAGGTGCAGCTGCAGGAGTCTGGA | Cad31 |
| | | | GGAGGATTGGTGCAGGCTGGGGCCTCTCTG | |
| | | | AGACTCTCCTGTGCAGCCTCTAGACGCACC | |
| | | | GGCAGTAGTCTTACCATGGGCTGGTTCCGC | |
| | | | CAGGCTCCAGGGAAGGAGCGTGAGTTTGTA | |
| | | | GCAGCTATTAGCCGGAGTGGTATTAGAACA | |
| | | | TACTACGCAGGCTTTGTGAAGGGCCGGTTC | |
| | | | ACCATCTCCAGAGACAACGCCAAGAACACG | |
| | | | CTCTATCTGCAAATGAACAGCCTGAAACCT | |
| | | | GAGGACACGGCCGTGTATTACTGTGCGGCT | |
| | | | AACGACAAAACATACGGTAGTGGTCTTGAC | |
| | | | GTCTACACAAGGCGACAAGACTATTACTAC | |
| | | | TGGGGCCCGGGGACCCAGGTCACCGTCTCC | |
| | | | TCAGCGGCCGCATACCCGTACGACGTTCCG | |
| | | | GACTACGGTTCCCACCACCATCACCATCAC | |
| | | | TAG | |
| 126 | Artificial | Protein | MAQVQLQESGGGLVQAGASLRLSCAASRRT | Cad31 |
| | | | GSSLTMGWFRQAPGKEREFVAAISRSGIRT | |
| | | | YYAGFVKGRFTISRDNAKNTLYLQMNSLKP | |
| | | | EDTAVYYCAANDKTYGSGLDVYTRRQDYYY | |
| | | | WGPGTQVTVSSAAAYPYDVPDYGSHHHHHH | |
| 127 | Artificial | DNA | ATGGCCCAGGTGCAGCTGCAGGAGTCTGGG | Cad41 |
| | | | GGAGGCTCGGTGCAGGCTGGGGGGTCTCTG | |
| | | | AAACTCTCCTGTGCAGCCTCTGGAACCGAA | |
| | | | AGCATTTTCATCGTGATGGGCTGGTACCGC | |
| | | | CAGGCTCCAGGGAAAGAGCGCGAATTGGTC | |
| | | | GCGGCAATGTCATTTGGTGGTGGAGCAAAT | |
| | | | GTTACAGACGGCGTGAGGGGCCGATTCACC | |
| | | | ATCTCCAGAGACCTTGACAAGAACACGGTA | |
| | | | GATCTGCAAATGAACAACCTAAAACCTGAG | |
| | | | GACACGGCCGTCTATTACTGTAATGCAGTC | |
| | | | CGGTGGGGCCCTCGTGACTACTGGGGTCAG | |
| | | | GGGACCCAGGTCACCGTCTCCTCAGCGGCC | |
| | | | GCATACCCGTACGACGTTCCGGACTACGGT | |
| | | | TCCCACCACCATCACCATCACTAG | |
| 128 | Artificial | Protein | MAQVQLQESGGGSVQAGGSLKLSCAASGTE | Cad41 |
| | | | SIFIVMGWYRQAPGKERELVAAMSFGGGAN | |
| | | | VTDGVRGRFTISRDLDKNTVDLQMNNLKPE | |
| | | | DTAVYYCNAVRWGPRDYWGQGTQVTVSSAA | |
| | | | AYPYDVPDYGSHHHHHH | |
| 129 | Artificial | DNA | ATGGCCCAAGTTCAACTGCAGGAGTCCGGC | NAAT3 |
| | | | GGGGGTGTAGTGCAGACTGGAGGCAGCTTG | |
| | | | ACGCTTTCCTGTAAGGCTTCACGTCGCACG | |
| | | | TCTGGATTTGCGATGGCCTGGTTCCGTCAG | |
| | | | GCCCCTGGGATGGAACGCGAGTTCGTTGCT | |
| | | | GGTATCGGGCGCACGGGTGATAACATCCAC | |
| | | | TACTTGGATTCTGTTAAAGGGCGTTTCGCT | |
| | | | ATTTCCCGTGACAACACAAAGAATACGTTA | |
| | | | TATCTTCAGATGAATAATTTGCAGCCAGAG | |
| | | | GATACAGCGGTCTATTACTGCTGCTTGGCT | |
| | | | AAAGCTGCTGATCCTTTTTGCGATCAAGGT | |
| | | | ACTCAGGTAACGGTCTCCTCAgcggccgca | |
| | | | tacccgtacgacgttccggactacggttcc | |
| | | | caccaccatcaccatcactag | |
| 130 | Artificial | Protein | MAQVQLQESGGGVVQTGGSLTLSCKASRRT | NAAT3 |
| | | | SGFAMAWFRQAPGMEREFVAGIGRTGDNIH | |
| | | | YLDSVKGRFAISRDNTKNTLYLQMNNLQPE | |
| | | | DTAVYYCCLAKAADPFCDQGTQVTVSSAAA | |
| | | | YPYDVPDYGSHHHHHH | |
| 131 | Artificial | DNA | ATGGCCCAATTGCAAGAATCAGGTGGCGGT | NAAT4 |
| | | | GTTGTTCAAACCGGAGGATCATTAACATTA | |
| | | | TCGTGCAAAGCGTCCCGTCGCACATCAGGC | |
| | | | TTCGCAATGGCGTGGTTTCGTCAGGCACCT | |
| | | | GGGATGGAGCGCGAGTTTGTGGCTGGGATT | |
| | | | GGGCGCACTGGTGATAATATCCATTACCTT | |
| | | | GATTCAGTAAAAGGACGTTTCACGATCTCA | |
| | | | CGTGATAATACTAAGAACACGCTTAGTTTA | |
| | | | CAGATGAACAACCTGAAACCGGAAGACACT | |
| | | | GCAGTGTACTACTGCGCAGCGAATCCTTGG | |
| | | | ATCAATACGGGAACGGGATGGAACTACTGG | |
| | | | GGACAAGGGACCCAAGTGACGGTATCTTCA | |
| | | | gcggccgcatacccgtacgacgttccggac | |
| | | | tacggttcccaccaccatcaccatcactag | |
| 132 | Artificial | Protein | MAQLQESGGGVVQTGGSLTLSCKASRRTSG | NAAT4 |
| | | | FAMAWFRQAPGMEREFVAGIGRTGDNIHYL | |
| | | | DSVKGRFTISRDNTKNTLSLQMNNLKPEDT | |
| | | | AVYYCAANPWINTGTGWNYWGQGTQVTVSS | |
| | | | AAAYPYDVPDYGSHHHHHH | |
| 133 | Artificial | DNA | ATGGCCCAGGTGCAGCTGCAGGAGTCTGGG | NAAT7 |
| | | | GGAGGCTTGGTGCAGCCTGGGGGGTCTCTG | |
| | | | AGACTCTCCTGTGCAGCCTCTGGAAGCATC | |
| | | | TTCAGTATCCCTGCCATGGGCTGGTACCGT | |
| | | | CAGGCTCCAGGGAAGCAGCGCGAGTTGGTC | |
| | | | GCAGTTATTACTAGAGATGGTAGCACGCAC | |
| | | | TATGCAGACTCCGTGAAGGGCCGATTCACC | |
| | | | ATCTCCAGAGACAACGCCAAAAACACGCTG | |
| | | | TATCTGCAAATGAACAGTCTGAAATCTGAG | |
| | | | GACACGGCCGTGTATTACTGTGCCAAACTG | |
| | | | GGTACTAGCCGATCGTATGACTACTGGGGC | |
| | | | CAGGGGACCCAGGTCACCGTCTCCTCAGCG | |
| | | | GCCGCATACCCGTACGACGTTCCGGACTAC | |
| | | | GGTTCCCACCACCATCACCATCACTAG | |
| 134 | Artificial | Protein | MAQVQLQESGGGLVQPGGSLRLSCAASGSI | NAAT7 |
| | | | FSIPAMGWYRQAPGKQRELVAVITRDGSTH | |
| | | | YADSVKGRFTISRDNAKNTLYLQMNSLKSE | |
| | | | DTAVYYCAKLGTSRSYDYWGQGTQVTVSSA | |
| | | | AAYPYDVPDYGSHHHHHH | |

1. An affinity construct comprising (1) at least one affinity molecule A capable of recognizing, or capable of binding to, or binding to, or being directed to, or being designed to bind to an insect-specific structure in and/or on a target insect, and (2) at least one affinity molecule B capable of binding to, or binding to, or being directed to, or being designed to bind to an insecticidal protein (toxin), wherein the at least one affinity molecule A and the at least one affinity molecule B are operably coupled.

2. The affinity construct according to claim 1, wherein the at least one affinity molecule A is different from the at least one affinity molecule B.

3. The affinity construct according to claim 1 or 2, wherein the at least one affinity molecule A comprises one or more binding sites (valences) for the same or different insect-specific structures in and/or on a target insect and wherein the at least one affinity molecule B comprises one or more binding sites (valences) for the same or different insecticidal protein (toxins).

4. The affinity construct according to any one of claims 1 to 3, wherein the at least one affinity molecule A specifically binds to a membrane-bound receptor of an inner organ of the target insect.

5. The affinity construct according to claim 4, wherein the at least one affinity molecule A specifically binds to a membrane-bound receptor of a digestive tract, of a reproductive organ, or of a nervous system.

6. The affinity construct according to claim 4, wherein the at least one affinity molecule A specifically binds to a fragment from an extracellular loop of Nutrient Amino Acid Transporter (NAAT) protein.

7. The affinity construct according to claim 4, wherein the at least one affinity molecule A specifically binds to a portion of an insect cadherin.

8. The affinity construct according to claim 4, wherein the at least one affinity molecule A specifically binds to a portion of integral membrane subunit (Vo) protein complex of the V-ATPase.

9. The affinity construct according to claim 4, wherein the at least one affinity molecule A specifically binds to an extracellular loop in ABCC1.

10. The affinity construct according to claim 4, wherein the at least one affinity molecule A specifically binds to an extracellular loop in ABCC2.

11. The affinity construct according to claim 4, wherein the at least one affinity molecule A specifically binds to an extracellular portion of venom dipeptidyl peptidase-4-like isoform X1 or peptide transporter family 1 isoform X1.

12. The affinity construct according to claim 1, wherein the insecticidal protein (toxin) is selected from the group consisting of crystal toxins (Cry and Cyt proteins), vegetative insecticidal toxins (Vip proteins), mosquitocidal toxins (Mtx proteins), binary toxins (Bin proteins), Tpp, Mpp, Gpp, App, Spp, Vpa, Vpb, Mcf, Pra, Prb, Xpp, Mpf toxins and secreted insecticidal toxins (Sip proteins), as well as fragments or multimers thereof.

13. The affinity construct according to claim 12, wherein the insecticidal protein (toxin) is selected from a group consisting of crystal toxins derived from Bacillus thuringiensis.

14. The affinity construct according to claim 1, wherein the at least one affinity molecule A and the at least one affinity molecule B are coupled by a linker sequence L.

15. The affinity construct according to any one of claims 1-3, wherein the at least one affinity molecule A and the at least one affinity molecule B are an affinity mediating molecule selected from the group consisting of a protein, carbohydrate, lipid or nucleotide, or a fragment, derivative or variant of any of these, wherein the at least one affinity molecule A and the at least one affinity molecule B are identical or different.

16. The affinity construct according to claim 15, wherein the protein is a non-antibody binding protein or a naturally occurring or engineered antibody or a fragment, derivative or variant thereof.

17. The affinity construct according to claim 16, wherein the non-antibody binding protein is selected from the group consisting of affimers (adhirons), affibodies, affilins, affitins, nanofitin, alphabodies (triple helix coiled coil), anticalins, lipocalins, avimers, DARPins (ankyrin repeat), fynomer, kunitz domain pepties, monobodies, adnectins, trinectins, nanoCLAMPs, cellulose/carbohydrate binding molecule (CBM) (for example, dockerins or lectins), centyrins, pronectins, and fibronectin or a fragment, derivative or variant of any of these.

18. The affinity construct according to claim 16, wherein the affinity molecule A comprises a naturally occurring or engineered antibody A or a fragment, derivative or variants thereof and the affinity molecule B comprises a naturally occurring or engineered antibody B or a fragment, derivative or variants thereof that are operably connected by the linker L.

19. The affinity construct according to claim 18, wherein the antibody A and antibody B, each is selected from the group consisting of a Fab fragment, a single heavy chain and a single light chain, a single chain variable fragment, a V_{H}H fragment, CDR3 region and a bispecific monoclonal antibody (diabody).

20. The affinity construct according to claim 18, wherein the antibody A and the antibody B, each comprises a single domain antibody or a fragment, derivative or variants thereof, operably connected by the linker L.

21. The affinity construct according to any one of claims 19-20, wherein the antibody A comprises one or more binding sites (valences) for the same membrane-bound receptor of a digestive tract, of a reproductive organ, or of a nervous system of an insect.

22. The affinity construct according to claim 21, wherein the antibody A comprises an amino acid sequence selected from any one of SEQ. ID. NOS. 74, 76, 78, 80, 82, 84, 86, 88, 90, 92, 94, 118, 120, 122, 124,126, 128, 130, 132, and 134.

23. The affinity construct according to claim 21, wherein the antibody A comprises a domain that specifically binds to a fragment from an extracellular loop of Nutrient Amino Acid Transporter (NAAT) protein.

24. The affinity construct according to claim 21, wherein the antibody A comprises a domain that specifically binds to a portion of an insect cadherin.

25. The affinity construct according to claim 21, wherein the antibody A comprises a domain that specifically binds to a portion of integral membrane subunit (Vo) protein complex of the V-ATPase.

26. The affinity construct according to claim 21, wherein the antibody A comprises a domain that specifically binds to an extracellular loop in ABCC1.

27. The affinity construct according to claim 21, wherein the antibody A comprises a domain that specifically binds to an extracellular loop in ABCC2.

28. The affinity construct according to claim 21, wherein the antibody A comprises a domain that specifically binds to an extracellular portion of venom dipeptidyl peptidase-4-like isoform X1 or peptide transporter family 1 isoform X1.

29. The affinity construct according to any one of claims 18 to 28, wherein the antibody B comprises one or more binding sites (valences) for the same or different insecticidal protein(s) (toxins).

30. The affinity construct according to claim 29, wherein the insecticidal protein (toxin) is selected from the group consisting of crystal toxins (Cry and Cyt proteins), vegetative insecticidal toxins (Vip proteins), mosquitocidal toxins (Mtx proteins), Tpp, Mpp, Gpp, App, Spp, Vpa, Vpb, Mcf, Pra, Prb, Xpp, Mpf toxins, binary toxins (Bin proteins), and secreted insecticidal toxins (Sip proteins), as well as fragments or multimers thereof.

31. The affinity construct according to claim 29, wherein the insecticidal protein (toxin) comprises a crystal protein toxin derived from Bacillus thuringiensis.

32. The affinity construct according to claim 29, wherein the antibody B comprises an amino acid sequence selected from any one of SEQ. ID. NOS. 68, 70 and 72.

33. The affinity construct according to any one of claims 18 to 32, wherein the linker L comprises an amino acid sequence selected from any one of SEQ. ID. NOS. 54, 56, 58, 60, 62 and 64.

34. The affinity construct according to claim 18, comprising an amino acid sequence of any one of SEQ. ID. NOS. 96, 98, 100, 102, 104, 106, 108, 110, 112, 114 and 116.

35. The affinity construct according to claim 18, comprising an amino acid sequence with at least about 70% sequence identity with any one of SEQ. ID. NOS. 96, 98, 100, 102, 104, 106, 108, 110, 112, 114 and 116.

36. The affinity construct according to claim 18, wherein the antibody A specifically binds to a fragment from the extracellular loop of an insect gut protein and antibody B specifically binds to a fragment of domain II of the Cry1F insect toxin.

37. The affinity construct according to claim 18, wherein the antibody A specifically binds to a fragment from an extracellular loop of an insect gut protein and antibody B specifically binds to a fragment of an insect toxin with at least 25% identity to Cry1F Domain II.

38. The affinity construct according to any one of claims 18, 36 or 37, wherein the antibody A specifically binds to a fragment from the extracellular loop of insect gut protein NAAT.

39. The affinity construct according to claim 38, wherein the antibody A specifically binds to a fragment from the extracellular loop of insect gut protein NAAT that has at least 50% similarity to the NAAT protein fragment the antibody A was raised against.

40. The affinity construct according to any one of claims 18, 36 or 37, wherein the antibody A specifically binds to a fragment from the extracellular loop of insect gut protein Cadherin.

41. The affinity construct according to any one of claim 40, wherein the antibody A specifically binds to a fragment from the extracellular loop of insect gut protein Cadherin that has at least 50% similarity to the Cadherin protein the antibody A was raised against.

42. A first recombinant nucleic acid construct, comprising a polynucleotide sequence encoding an amino acid sequence of the affinity construct of any one of the claims 18-41.

43. The first recombinant nucleic acid construct of claim 42, comprising a nucleic acid sequence that is at least about 70% identical with any one of SEQ. ID NOS. 67, 69, 71, 73, 75, 77, 79, 81, 83, 85, 87, 89, 91, 93, 117, 119, 121, 123, 125, 127, 129, 131, 133.

44. The first recombinant nucleic acid construct of claim 42, comprising a nucleic acid sequence that is at least about 70% identical with any one of SEQ. ID NOS. 73, 75, 77, 79, 81, 83, 85, 87, 89, 91, 93, 117, 119, 121, 123, 125, 127, 129, 131, 133.

45. The first recombinant nucleic acid construct of claim 44, further comprising a nucleic acid sequence that is at least about 70% identical to any one of SEQ. ID NOS. 67, 69, 71.

46. The first recombinant nucleic acid construct of any one of claims 43 to 45, further comprising a linker sequence that is at least about 70% identical to any one of SEQ. ID NOS. 55, 57, 59. 61, 63, and 65.

47. The first recombinant nucleic acid construct of claim 42, wherein the nucleic acid sequence is at least about 70% identical with any one of SEQ. ID. NOS. 95, 97, 99, 101, 103, 105, 107, 109, 111, 113 and 115.

48. The first recombinant nucleic acid construct of any one of claims 42 and 57, wherein the sequence encoding the amino acid sequence is codon optimized for expression in a selected host cell.

49. The first recombinant nucleic acid construct of claim 48, wherein the selected host cell is a yeast cell, a bacterial cell, or a plant cell.

50. The first recombinant nucleic acid construct of claim 42, further comprising a promoter operably linked to the polynucleotide sequence.

51. The first recombinant DNA construct of claim 50, wherein the promoter comprises a constitutive promoter, an inducible promoter, a plant specific promoter, a plant tissue specific promoter, a CaMV promoter or a microbial promoter.

52. A transgenic host cell comprising the first recombinant DNA construct of any one of claims 42-51.

53. The transgenic host cell of claim 52, further comprising one or more nucleic acid sequences, each encoding an insecticidal protein (toxin) that specifically binds to or can be targeted to bind to at least one domain in the at least one affinity molecule B of the affinity construct.

54. The transgenic host cell of any one of claims 52 and 53, wherein the transgenic host cell is a yeast cell, a bacterial cell, or a plant cell.

55. An insecticidal composition comprising the affinity construct according to any one of claims 1 to 41 and at least one insecticidal protein (toxin), wherein the at least one insecticidal protein (toxin) can specifically bind to or can be targeted to bind to at least one domain in the at least one affinity molecule B of the affinity construct.

56. The insecticidal composition of claim 55, further comprising a carrier.

57. The insecticidal composition of claim 56, wherein the carrier may be any one of a powder, a dust, pellets, granules, spray, emulsion, colloid or a solution.

58. The insecticidal composition in any one of claims 56-57, further comprising an insect food source.

59. The insecticidal composition of any one of claims 56-58, wherein the insecticidal composition is specifically toxic to one or more insect pests including insects from orders Isoptera, Blattodea, Orthoptera, Phthiraptera, Thysanoptera, Hemiptera, Hymenoptera, Siphonaptera, Diptera, Coleoptera and Lepidoptera.

60. The insecticidal composition of any one of claims 56-59, wherein the insecticidal composition is specifically toxic to one or more insect pests selected from a group consisting of Fall armyworm (FAW, Spodoptera frugiperda), Corn earworm (Helicoverpa zea, CEW) and Diamond back moth (DBM, Plutella xylostella).

61. The insecticidal composition of any one of claims 56-60, wherein the insecticidal composition is specifically toxic to two or more insect pests.

62. Use of the insecticidal composition in any one of claims 55-61 for preventing damage to a plant, plant part or plant seed by one or more insect pest(s).

63. A method of preventing damage to a plant, a plant part or plant seed by one or more insect pests, comprising contacting the insect pests with the insecticidal composition as in any one of claims 56-62.

64. The method of preventing damage to a plant, a plant part or plant seed by one or more insect pests of claim 63, wherein the insect pests include insects selected from the orders Isoptera, Blattodea, Orthoptera, Phthiraptera, Thysanoptera, Hemiptera, Hymenoptera, Siphonaptera, Diptera, Coleoptera and Lepidoptera.

65. An insecticidal kit, comprising the insecticidal composition in any one of claims 55-64.

66. The insecticidal kit of claim 65, further comprising the host cell of any of claims 46-48.

67. The insecticidal kit of any one of claim 65 or 66, further comprising the instructions for making an insecticidal preparation and using the kit to prevent damage to a plant, plant part or plant seed by one or more insect pest(s) of claim 58.

68. A method of protecting a plant or plant parts or plant seeds against one or more insect pest(s) comprising:
co-expressing the affinity construct according to any one of claims 18 to 41 together with one or more insecticidal protein(s) (toxin(s)) in a plant, plant parts or plant seeds, wherein the one or more insecticidal protein(s) (toxin(s)) can specifically bind to or can be targeted to bind to at least one domain in the at least one affinity molecule B of the affinity construct.

69. A method of protecting a plant or plant parts or plant seeds against one or more insect pest(s) comprising:
a. expressing the affinity construct according to any one of claims 18 to 41 in a plant, plant parts or plant seeds and
b. applying the one or more insecticidal protein(s) (toxin(s)) to the plant, plant parts or plant seeds, wherein the one or more insecticidal protein(s) (toxin(s)) can specifically bind to or can be targeted to bind to at least one domain in the at least one affinity molecule B of the affinity construct.

70. A method of protecting a plant or plant parts or plant seeds against one or more insect pest(s) comprising:
a. applying the affinity construct according to any one of claims 18 to 41 to the plant, plant parts or plant seeds, wherein said affinity construct is expressed in one or more microorganism(s) and is applied to said plant, plant parts or plant seeds either in purified form or by applying the microorganism(s) expressing the affinity construct; and
b. expressing the one or more insecticidal protein(s) (toxin(s)) in the plant, plant part or plant seed, wherein the one or more insecticidal protein (toxin) can specifically bind to or can be targeted to bind to at least one domain in the at least one affinity molecule B of the affinity construct.

71. A method of protecting a plant or plant parts or plant seeds against one or more insect pest(s) comprising:
a. (co-)expressing the affinity construct according to any one of claims 18 to 41 and one or more insecticidal protein(s) (toxin(s)) in one or more microorganisms.
b. applying the one or more microorganisms (co-)expressing the affinity construct and the one or more insecticidal protein(s) (toxin(s)) either in purified form or together with the respective culture medium/media to a plant, plant parts or plant seeds, wherein the one or more insecticidal protein (toxin) can specifically bind to or can be targeted to bind to at least one domain in the at least one affinity molecule B of the affinity construct, wherein ingestion of the microorganism or culture medium/media by the insect pest causes morbidity to or mortality of the insect pest(s).

72. A method of producing a plant or a microorganism comprising the affinity construct according to any one of claims 18 to 41 and one or more insecticidal protein(s) (toxin(s)), the method comprising co-expressing in a plant or microorganism the affinity construct according to any one of claims 18 to 41 and one or more insecticidal protein(s) (toxin(s)), wherein said one or more insecticidal protein(s) (toxin(s)) correspond(s) to the insecticidal protein(s) (toxin(s)) which the at least one affinity molecule B is capable of binding to, or is binding to, or is being directed to, or is being designed to bind to.

73. A method of protecting a plant or plant parts or plant seeds against one or more insect pest(s) comprising the step of transforming the plant or microorganism with one or more nucleic acid molecules encoding the affinity construct according to any one of claims 18 to 41, and one or more nucleic acid molecules encoding the insecticidal protein(s) (toxin(s)), wherein said one or more insecticidal protein(s) (toxin(s)) correspond(s) to the insecticidal protein(s) (toxin(s)) which the at least one affinity molecule B is capable of binding to, or is binding to, or is being directed to, or is being designed to bind to.

74. A method of producing an insecticidal formulation comprising the affinity construct according to any one of claims 1 to 41 and one or more insecticidal protein(s) (toxin(s)), the method comprising formulating the affinity construct according to any one of claims 1 to 41 and one or more insecticidal protein(s) (toxin(s)) as insecticidal formulation, wherein said one or more insecticidal protein(s) (toxin(s)) correspond(s) to the insecticidal protein(s) (toxin(s)) which the at least one affinity molecule B is capable of binding to, or is binding to, or is being directed to, or is being designed to bind to, and wherein said affinity construct and said one or more insecticidal protein(s) (toxin(s)) are expressed in one or more microorganism.

75. The affinity constructs according to any one of claims 1-41, wherein the affinity construct enhances the activity of Cry1F against a natural target of Cry1F as indicated by a mortality assay.

76. The affinity construct according to claim 75, wherein the natural target is FAW.

77. The affinity construct according to claim 75 or 76 wherein the affinity construct comprises any one of BsNbs number: 10, 18, 39, 62, 64, 76, 100 or 111.

78. The affinity construct according to claim 76 wherein the natural target is DBM.

79. The affinity construct according to claim 75 or 78 wherein the affinity construct comprises any one of BsNbs number: 4, 100, or 111.

80. The affinity construct according to any one of claims 1-41, wherein the affinity construct expands the activity of Cry1F to previously non-susceptible insects as determined (or as measured) by a mortality assay.

81. The affinity construct according to claim 80, wherein the non-susceptible insect is CEW and the affinity construct comprises any one of BsNbs number: 21, 100 or 108.

82. The affinity constructs according to any one of claims 1-41, wherein the affinity construct enhances the activity of Cry1Ab against a natural target of Cry1Ab.

83. The affinity construct according to claim 82, wherein the natural target is VBC.

84. The affinity construct of claim 83, wherein the affinity construct comprises any one of BsNbs number: 4 or 108.

85. The affinity construct according to claim 82, wherein the natural target is SCB.

86. The affinity construct according to claim 85, wherein the affinity construct comprises any one of BsNbs number: 10 or 18.

87. The affinity construct according to any one of claims 1-41, wherein the affinity construct expands the activity of Cry1Ab previously non-susceptible insects as determined (or as measured) by a mortality assay.

88. The affinity construct according to claim 87, wherein the non-susceptible insect is FAW.

89. The affinity construct according to claim 87 or 88 wherein the affinity construct comprises any one of BsNbs number: 4, 15 or 76.

90. The affinity construct according to claim 87, wherein the non-susceptible insect is CEW.

91. The affinity construct according to claim 90, wherein the affinity construct is a BsNbs number: 4.

92. The affinity construct according to any one of claims 1-41, wherein the affinity construct enhances the activity of Cry1Ac against a natural target of Cry1Ac as indicated by a mortality assay.

93. The affinity construct according to claim 92, wherein the natural target is FAW.

94. The affinity construct according to claim 92 or 93 wherein the affinity construct comprises any one of BsNbs number: 10, 15, 39, 62, 64, 76, 100 or 111.

95. The affinity construct according to claim 92 wherein the natural target is DBM.

96. The affinity construct according to claim 92 or 95 wherein the affinity construct comprises any one of BsNbs number: 4, 21, 100, 111, 112.

97. The affinity construct according to claim 92 wherein the natural target is BCW.

98. The affinity construct according to claim 92 or 97 wherein the affinity construct comprises any one of BsNbs number: 10, 64 or 100.

99. The affinity construct according to any one of claims 1-41, wherein the affinity construct expands the activity of Cry1F previously non-susceptible insects as determined (or as measured) by a mortality assay.

100. The affinity construct according to claim 99, wherein the non-susceptible insect is ECB.

101. The affinity construct according to claim 100 and the affinity construct is BSNb 18.


SEQUENCE LISTING (134 sequences)

SEQ ID NO: 1 - DNA, 5592 bp, Spodoptera frugiperda
gacattctgtggtgaaaacattttttatttatttttttctagtggtttgtgggtacagtgtaaacattttggaatattgttaaagatttcggaatattgttaaagtattgacagataaagctgtaacatcactagagaagtgagaactgcaagatcatgagatggcggtcgatgtgcgaatactgacagcaacattgctggtactcaccactgctacagcacagcgagatcgatgtggctacatggtagaaatacccagaccagacaggcctgacttcccacctcaaaattttgacggtttaacatgggctcagcagccactattaccagctgaggatcgagaagaggtctgcctcaatgactatgaacctgatccctggagcaacaaccatggtgaccagagaatttacatggaggaggagatcgaaggtcccgtagtcattgcgaaaattaactaccaaggaaacacccctcctcaaataagattaccttttcgtgttggtgcagcccacatgcttggagcagaaattcgtgaatatcctgacgcaactggagactggtatcttgtaattactcaaaggcaggactatgaaactcctgatatgcagagatacacgttcgatgtgagtgtggaaggccagtcgctggttgtaacggtgaggctggatattgtgaacatcgacgacaatgcgcccatcattgagatgttagagccttgcaacttaccggaacttgttgaaccccatgttacagaatgtaaatatatcgtgtccgacgcagacggtctgatcagtacaagtgttatgagttatcatatagacagcgagagaggagacgaaaaagtattcgaactgatcagaaaagattatccgggcgattggacgaaggtgtatatggttcttgaattgaaaaaatctcttgattacgaagagaatcctctacacatattcagagtcacggcttctgattccttaccaaacaataggaccgtggtcatgatggttgaagtagagaacgtggaacatagaaatcctcggtggatggagatctttgctgtgcaacagtttgatgaaaaacaggcgaaatcgttcacagtgcgagctattgatggcgacacgggaatcaataaacctatattctatcgtatagaaactgaagatgaagacaaagagttcttcagcattgagaacataggggaaggcagagacggtgccagattccacgtggctcctatagacagagactacctgaaaagggatatgtttcatataagaataattgcatataaacaaggtgataatgacaaagaaggtgaatcatcgttcgagacctcagcaaatgtgacgattataattaacgatataaatgatcagaggccagaacccttccataaagaatacacgatctccataatggaagaaactgcgatgaccttagatttgcaagagtttggtttccatgaccgtgacattggtccccacgctcagtacgacgttcacttagagagtatacagccagagggggcccataccgctttctacatcgcccctgaagaaggttaccaggcccagtctttcaccataggtactagaatccataacatgttggattatgaagatgacgactacagaccaggaataaagctaaaggcagtagcaattgacagacacgataacaatcacattggggaagcaattattaacattaaccttatcaattggaatgatgagctacctatattcgacgaggacgcctacaacgtgacatttgaggagacggtcggtgatggcttccacattggtaaataccgggctaaagacagagacatcggtgacatagtcgagcactcgatattgggcaacgctgcaaacttcctgagaattgacatagatactggagatgtgtacgtgtcacgggacgattactttgattatcaaagacagaacgaaatcatagttcagattctggctgttgatacactaggtttacctcagaacagggctaccacacagctcacgatatttttggaagacatcaacaacacgccacctatactgcgactgccacgttccagtccaagtgtagaagagaacgttgaagtcgggcacccgattaccgaggggctaacggcgacagacccagacaccacagccgatttacacttcgagatcgattgggacaattcttacgctacgaagcagggcaccaatggacccaacactgcagactaccacggatgcgtagaaatcctgacggtatacccagatcctgacaatcacgggagagctgagggtcacttggtggcacgtgaggtcagtgatggcgtgaccatcgattacgagaagtttgaggtgctgtacctcgtcgtcagggtgatagatcgcaacactgtcattggccctgattatgacgaagcaatgctgacggtgacgataatcgatatgaacgacaactggccgatatgggccgacaacacgctgcagcagacactgcgcgtgcgcgagatggccgacgaaggagtcatcgtcggtacactgctcgccaccgacttggatggccctctctacaaccgagtccgctacaccatggtccccatcaaggacactcctgatgacctaatagcgatcaactacgtcaccggtcagctgactgtgaacaaggggcaagcaattgacgcagatgatccacctcgcttctacctgtattacaaggtcactgccagcgataagtgctctcttgacgagttcttccctgtgtgcccacctgaccccacttactggaataccgagggagagatagcgatcgcgataaccgatacgaacaacaaaattccacgcgcggaaacagatatgttccctagtgaaaagcgcatctatgagaacacacccaatggtaccaagatcacgacgatcatcgctagtgaccaggacagagatcgaccaaataacgcgctgacgtacagaatcaactacgcattcaaccacaggctggagaacttcttcgcagtggaccctgatactggtgaactgtttgtccacttcaccactagcgaagtgttggacagagacggagaggaaccggagcataggatcatcttcaccatcgtcgataacttggaaggcgctggagatggcaatcagaacacaatctccacggaggtgcgtgttatactgcttgatataaacgacaataagccggaactaccaattcctgatggcgaattttggaccgtttccgaaggtgaagtggagggaaaacgcattccaccagagattcacgcacacgacagagatgaaccattcaacgacaactctcgcgtgggatatgaaattcgatcgatcaaattgatcaatagagacatcgagcttcctcaagatccattcaaaataataacgattgatgatctcgatacctggaaattcgttggagagttggagactaccatggaccttagaggatactggggaacctatgatgtcgagatacgtgcgtttgaccacggtttcccgatgctggattcattcgagacctaccaactaaccgtcaggccatacaacttccattcaccggtgtttgtgttcccaactcctggctcaaccatcaggctttctagggagcgtgctatagtcaatggtatgctggctctggctaatatcgcgagcggagagttcctcgacagactctctgccactgatgaagatgggctacacgcaggcagagtaactttctccatagctggaaacgatgaagctgcggaatatttcaatgtgttgaacgacggtgacaactcagcaatgctcacgctgaagcaagcattgcccgctggcgtccagcagtttgagttggttattcgggccacggacggcgggacggagccgggacctaggagtaccgactgctccgtcactgtggtgtttgtgatgacgcagggagaccccgtgttcgacgacaacgcagcttctgtccgcttcgttgaaaaggaagctggtatgtcggaaaagtttcagctgcctcaggccgatgaccccaaaaactacaggtgtatggacgactgccataccatctactactctatcgttgatggcaacgatggtgaccacttcgccgtggagccggagactaacgtgatctatttgctgaagccgctggaccgcagccaacaggagcagtacagggtcgtggtggcggcttccaacacgcctggcggcacctccaccttgtcctcctcactcctcaccgtcaccatcggcgttcgagaagcaaaccctagaccgatcttcgaaagtgaattttacacagctggcgtcttacacaccgatagcatacacaaggagctcgtttacctggcggcaaaacattcagaagggcttcctatcgtctactcgatagatcaagaaaccatgaaaatagacgagtcgttgcaaacagttgtggaggacgccttcgacattaactctgcaaccggagtcatatcgctgaacttccagccaacatctgtcatgcacggcagtttcgacttcgaggtggtggctagtgacacgcgtggagcgagtgatcgagcaaaagtgtcaatttacatgatatcgactcgcgttagagtagccttcctgttctacaacacggaagctgaagttaacgagagaagaaatttcattgcacaaacgttcgccaacgcgtttggtatgacatgtaacatagacagcgtgctgccggctaccgacgccaacggcgtgattcgcgaggggtacacagaactccaggctcacttcatacgagacgaccagccggtgccagccgactatattgagggattatttacggaactcaatacattgcgtgacatcagagaggtactgagtactcagcaattgacgctactggactttgcggcgggagggtcggcagtgctgcccggcggagagtacgcgctagcggtgtacatcctcgccggcatcgcagcgttactcgccgtcatctgtctcgctctcctcatcgctttcttcattaggaaccgaacactgaaccggcgcatcgaagccctcacaatcaaagatgttcctacggacatcgagccaaaccacgcgtcagtagcagtgctaaacattaacaagcacacagaacctggttccaatcccttctataacccggatgttaagacacctaacttcgacactataagcgaagtatccgatgacctgcttgatgtcgaagacttggaacagtttggaaaggattacttcccacccgaaaacgaaattgagagcctgaattttgcacgtaaccccatagcgacacacgggaacaactttggcgtaaactcaagcccctccaacccagagttctccaactcccagtttagaagttaaactaaatacacttttatcacttgcatagacttatgtatttaataattttacattttttacattaaatataaatgttttatatgtaataatagtgtgataaaatgtacgtaacaatcaacatagctgttgtaggttcgtaaataacatactcgtaatgtataagtgttatgtttatatatagaaataaaaatattaaatattaaaaaaaaaaaaaaaaaaaaaaaaa

SEQ ID NO: 2 - protein, 1742 aa, Spodoptera frugiperda
MAVDVRILTATLLVLTTATAQRDRCGYMVEIPRPDRPDFPPQNFDGLTWAQQPLLPAEDREEVCLNDYEPDPWSNNHGDQRIYMEEEIEGPVVIAKINYQGNTLIGVSVVLPPQIRLPFRVGAAHMLGAEIREYPDATGDWYLVITQRQDYETPDMQRYTFDVSVEGQSLVVTVRLDIVNIDDNAPIIEMLEPCNLPELVEPHVTECKYIVSDADGLISTSVMSYHIDSERGDEKVFELIRKDYPGDWTKVYMVLELKKSLDYEENPLHIFRVTASDSLPNNRTVVMMVEVENVEHRNPRWMEIFAVQQFDEKQAKSFTVRAIDGDTGINKPIFYRIETEDEDKEFFSIENIGEGRDGARFHVAPIDRDYLKRDMFHIRIIAYKQGDNDKEGESSFETSANVTIIINDINDQRPEPFHKEYTISIMEETAMTLDLQEFGFHDRDIGPHAQYDVHLESIQPEGAHTAFYIAPEEGYQAQSFTIGTRIHNMLDYEDDDYRPGIKLKAVAIDRHDNNHIGEAIININLINWNDELPIFDEDAYNVTFEETVGDGFHIGKYRAKDRDIGDIVEHSILGNAANFLRIDIDTGDVYVSRDDYFDYQRQNEIIVQILAVDTLGLPQNRATTQLTIFLEDINNTPPILRLPRSSPSVEENVEVGHPITEGLTATDPDTTADLHFEIDWDNSYATKQGTNGPNTADYHGCVEILTVYPDPDNHGRAEGHLVAREVSDGVTIDYEKFEVLYLVVRVIDRNTVIGPDYDEAMLTVTIIDMNDNWPIWADNTLQQTLRVREMADEGVIVGTLLATDLDGPLYNRVRYTMVPIKDTPDDLIAINYVTGQLTVNKGQAIDADDPPRFYLYYKVTASDKCSLDEFFPVCPPDPTYWNTEGEIAIAITDTNNKIPRAETDMFPSEKRIYENTPNGTKITTIIASDQDRDRPNNALTYRINYAFNHRLENFFAVDPDTGELFVHFTTSEVLDRDGEEPEHRIIFTIVDNLEGAGDGNQNTISTEVRVILLDINDNKPELPIPDGEFWTVSEGEVEGKRIPPEIHAHDRDEPFNDNSRVGYEIRSIKLINRDIELPQDPFKIITIDDLDTWKFVGELETTMDLRGYWGTYDVEIRAFDHGFPMLDSFETYQLTVRPYNFHSPVFVFPTPGSTIRLSRERAIVNGMLALANIASGEFLDRLSATDEDGLHAGRVTFSIAGNDEAAEYFNVLNDGDNSAMLTLKQALPAGVQQFELVIRATDGGTEPGPRSTDCSVTVVFVMTQGDPVFDDNAASVRFVEKEAGMSEKFQLPQADDPKNYRCMDDCHTIYYSIVDGNDGDHFAVEPETNVIYLLKPLDRSQQEQYRVVVAASNTPGGTSTLSSSLLTVTIGVREANPRPIFESEFYTAGVLHTDSIHKELVYLAAKHSEGLPIVYSIDQETMKIDESLQTVVEDAFDINSATGVISLNFQPTSVMHGSFDFEVVASDTRGASDRAKVSIYMISTRVRVAFLFYNTEAEVNERRNFIAQTFANAFGMTCNIDSVLPATDANGVIREGYTELQAHFIRDDQPVPADYIEGLFTELNTLRDIREVLSTQQLTLLDFAAGGSAVLPGGEYALAVYILAGIAALLAVICLALLIAFFIRNRTLNRRIEALTIKDVPTDIEPNHASVAVLNINKHTEPGSNPFYNPDVKTPNFDTISEVSDDLLDVEDLEQFGKDYFPPENEIESLNFARNPIATHGNNFGVNSSPSNPEFSNSQFRS

SEQ ID NO: 3 - DNA, 5193 bp, Helicoverpa armigera
atggcagtcgacgtgagaatattcacggcagcggtttttatactcgctgctcacttcactttcgcacaagattgtagctacatggtagcaatacccagaccagagcgaccagattttccaagtcaaaatttcgatggaataccatggagtcagtatcccttgataccagtggagggtagagaagacgtgtgtatgaacgagttcgagccaggtaaccaaaaccctgttaccgtcatcttcatggaagaggagatcgaaggggatgtggccatcgcacggctcaactatcgaggtaccaatactccgaccattgtatctccatttagctttggtacttttaacatgttggggccggtcatacgtagaatacctgagaatggtggtgactggcatctcgtcattacacagagacaggactacgagacaccaggtatgcagcagtacatcttcgacgtgagggtagacgacgaacccctggtggccacggtcatgcttctcatcgtcaacattgatgacaacgatcctatcatacagatgttcgagccttgtgatattccggaacgcggtgaaacaggcatcacatcatgcaagtatacagtcagcgatgctgacggtgagatcagtactcgcttcatgaggttcgaaatttcaagcgatcgagacgatgacgaatatttcgaactcgtcagggaaaatatacaaggacagtggatgtatgttcatatgagagttcacgtcaaaaaacctcttgactacgaggaaaacccgctacatttgtttagagttacagcttatgattccctaccaaacacacatacagtaacaatgatggtgcaagtagagaacgttgagaacagaccgccgcgatgggtggagatatttgctgtccagcagttcgatgagaagacggagcggtccttcagggttcgagccatcgatggtgatacaggaatcgataaacctatcttctataggatcgaaacagaagaaggagaggaaaacttgttcagcattcaaacaatggaaggtggtcgagaaggagcttggtttaacgttgctccaatagacagagacactcttgagaaggaagttttccacgtgtccataatagcgtacaaatatggtgataatgacgtggaaggcagttcgtcgttccagtcgaaaaccgatgtggtcatcatcgtgaacgatgtcaatgatcaggctccgttgcctttccgggaagagtattccattgaaattatggaggaaactgcgatgacactgaacttagaagactttgggttccacgatagagatcttggtcctcacgcccaatacacagtgcacctagagagcatccatcccccccgagctcacgaggcgttctacatagcaccggaggtgggctaccagcgccagtccttcatcatgggcacgcagaaccatcacatgctggactttgaagtgccggagttccagaatatacaactgagggctatagcgatagacatggacgatcccaaatgggttggtattgcgataatcaacattaaactgatcaactggaacgatgagctgccgatgttcgagagtgacgtgcaaaccgtcagcttcgacgagacagagggcgctggcttctatgtggccactgttgtggcgaaggaccgggatgttggtgataaagtcgaacactctctaatgggtaacgcagtaagctacctgaggatcgacaaggaaaccggcgagatattcgtcacagaaaacgaagctttcaactatcacagacagaacgaactctttgtgcagatacgagctgatgacacattaggcgagccatacaacaccaacactacccagttggtgatcaagctgcgggatattaacaacactcctcctacgctcagactgcctcgcgccactccgtcagtggaagagaacgtgcccgacgggtttgtgatccccacacaactgaacgccacggaccccgacactacagccgagctgcgcttcgagatcgactgggagaactcctatgccaccaagcagggacggaatactgactctaaggagtatataggttgtatagaaatcgagacgatatacccgaatataaaccagcgcggcaacgccatcggccgcgtggtggtgcgagagatccgggacggcgtcaccatagactatgagatgtttgaagtgctatatctgacggtcattgtgagggatctcaacactgttattggggaagaccatgatatttccacattcacaatcacaataatagacatgaacgacaaccctcccctgtgggtggaaggcaccctcactcaagagttccgcgtgcgagaggtcgcagcctcaggagtcgttataggatccgtactggctactgatatcgacggaccgctgtataatcaagtgcagtatactataactcccagactcgatactccagaagacctagtggacatagacttcaacacgggtcagatctccgtgaagttacaccaagctatagatgcagacgagccgccgcgtcagaacctctactacaccgtcatagctagtgacaagtgtgacctcctcactgtcactgggtgtcctcctgaccctacctactttgggacgccgggagagatcacgatccacataacggacacgaacaacaaggtgcctcaagtggaagacgacaagttcgaggcgacggtgtacatctacgagggcgcggacgacggagaacacgtcgtgcagatctacgccagcgatctcgatagagatgaaatctaccacaaagtgagctaccagatcaactacgcgatcaactcccgtctccgcgacttcttcgagatggacctggagacaggcctggtgtacgtcaacaacaccgccggcgagctgctggacagggacggcgacgagcccacacatcgcatcttcttcaatgtcatcgataacttctatggagaaggagatggcaaccgcaatcagaacgagacacaagtattggtagtattgctggacatcaacgacaactatccagaactgcctgaaacaatcccatgggctatctctgagagcttagagcagggtgagcgagtaccgccagaaatcttcgcccgggaccgcgatgaacccggaacagacaactcccgcgtcgcctacgccatcaccggtcttaccagcaccgaccgggacatacaagtgcctgatctcttcaacatgatcaccatagagagggacaggggaattgatcaaactggaatacttgaggcagctatggatttgcggggctattggggcacttatgaaattgatatacaggcgtacgaccatggcatacctcgaaggatttcaaatcagaagtacccgctggtcatcagaccttacaacttccacgacccagtgttcgtgttccctcaacctggatctactatcagactggcaaaggagcgagcagtagtcaacggtatactggccacagtggacggcgaatttctggacagaatcgtcgccaccgacgaagatggtttagaagctggacttgttacattctctatcgcgggagatgatgaagatgctcagttcttcgacgtgttgaacgatggagtgaactcgggcgctctcaccctcacgcggctcttccctgaagatttccgagagttccaggtgacgattcgtgctacggacggtggaactgagcctggtccaaggagtacggactgtgcggtgaccgtagtgtttgttcccacacagggagagcccgtgttcgaggaaagtacctacacggtcgcttttgttgaaaaagatgagggtatggaggagagggcagaattacctcgcgcctcagatccgaggaacatcatgtgtgaagatgactgtcacgacacctattacagcattgttggaggcaattcgggtgaacacttcagagtagaccctcgtaccaacgtgctgaccctcgtgaagccgctggaccgctccgaacaggagacacacaccctcatcatcggagccagcgacactcccaacccggccgccgtcctgcaggcttctacactcactgtcactgttaatgttcgagaagcgaacccgcgaccagtgttccagagagcactctacacagctggcatctctgctggcgatttcatcgaaagaaatctgctgactgtagtagcgacacattcagaaggtctgcccatcacttacactctgatacaagagtccatggaagcagaccccacactcgaagctgttcaggagtcagccttcatcctcaaccctgagactggagtcctgtcactcaacttccagccaaccgccgccatgcatggcatgtttgagttcgaagtcgaagccactgattcaaggagagaaactgcccgcacggaagtgaaggtgtacgtgatatcggaccgcaaccgagtgttcttcacgttcaataacccgctgcctgaagtcacaccccaggaagatttcatagcggagacgttcacggcattcttcggcatgacgtgcaacatcgaccagacgtggtgggccagcgaccccgtcaccggcgccaccagggacgaccagactgaagtcagggctcatttcatcagggacgacttacccgtgcctgctgaggagattgaacagttacgcggtaacccaactctagtaaatagcatccaacgagccctagaagaacagaacctgcagctggccgacctgttcacgggcgagacgcccatcctgggcggtgacgcgcaggcgcgagctctgtacgcgctggcggcggtggctgctgcactcgcgctgattgtagttgtgctgcttattgtgttctttgttaggactaggactctgaaccggcgcttgcaagctctatccatgaccaagtacagttcgcaagactcggggctgaaccgcgtgggtctggcggcgccgggcaccaacaagcacgccgtcgagggctccaaccccatctggaacgaaacgttgaaggctccagactttgatgctcttagtgagcagtcatacgactcagacctgatcggcatcgaagacttgccgcagttcaggaacgactacttcccgcctgaagagggcagctccatgcgaggagtcgtcaatgaacacgtgcctgaatcaatagcgaaccataacaacaacttcgggttcaactctactcccttcagcccagagttcgcgaacacacagttcggaagataa

SEQ ID NO: 4 - protein, 1730 aa, Helicoverpa armigera
MAVDVRIFTAAVFILAAHFTFAQDCSYMVAIPRPERPDFPSQNFDGIPWSQYPLIPVEGREDVCMNEFEPGNQNPVTVIFMEEEIEGDVAIARLNYRGTNTPTIVSPFSFGTFNMLGPVIRRIPENGGDWHLVITQRQDYETPGMQQYIFDVRVDDEPLVATVMLLIVNIDDNDPIIQMFEPCDIPERGETGITSCKYTVSDADGEISTRFMRFEISSDRDDDEYFELVRENIQGQWMYVHMRVHVKKPLDYEENPLHLFRVTAYDSLPNTHTVTMMVQVENVENRPPRWVEIFAVQQFDEKTERSFRVRAIDGDTGIDKPIFYRIETEEGEENLFSIQTMEGGREGAWFNVAPIDRDTLEKEVFHVSIIAYKYGDNDVEGSSSFQSKTDVVIIVNDVNDQAPLPFREEYSIEIMEETAMTLNLEDFGFHDRDLGPHAQYTVHLESIHPPRAHEAFYIAPEVGYQRQSFIMGTQNHHMLDFEVPEFQNIQLRAIAIDMDDPKWVGIAIINIKLINWNDELPMFESDVQTVSFDETEGAGFYVATVVAKDRDVGDKVEHSLMGNAVSYLRIDKETGEIFVTENEAFNYHRQNELFVQIRADDTLGEPYNTNTTQLVIKLRDINNTPPTLRLPRATPSVEENVPDGFVIPTQLNATDPDTTAELRFEIDWENSYATKQGRNTDSKEYIGCIEIETIYPNINQRGNAIGRVVVREIRDGVTIDYEMFEVLYLTVIVRDLNTVIGEDHDISTFTITIIDMNDNPPLWVEGTLTQEFRVREVAASGVVIGSVLATDIDGPLYNQVQYTITPRLDTPEDLVDIDFNTGQISVKLHQAIDADEPPRQNLYYTVIASDKCDLLTVTGCPPDPTYFGTPGEITIHITDTNNKVPQVEDDKFEATVYIYEGADDGEHVVQIYASDLDRDEIYHKVSYQINYAINSRLRDFFEMDLETGLVYVNNTAGELLDRDGDEPTHRIFFNVIDNFYGEGDGNRNQNETQVLVVLLDINDNYPELPETIPWAISESLEQGERVPPEIFARDRDEPGTDNSRVAYAITGLTSTDRDIQVPDLFNMITIERDRGIDQTGILEAAMDLRGYWGTYEIDIQAYDHGIPRRISNQKYPLVIRPYNFHDPVFVFPQPGSTIRLAKERAVVNGILATVDGEFLDRIVATDEDGLEAGLVTFSIAGDDEDAQFFDVLNDGVNSGALTLTRLFPEDFREFQVTIRATDGGTEPGPRSTDCAVTVVFVPTQGEPVFEESTYTVAFVEKDEGMEERAELPRASDPRNIMCEDDCHDTYYSIVGGNSGEHFRVDPRTNVLTLVKPLDRSEQETHTLIIGASDTPNPAAVLQASTLTVTVNVREANPRPVFQRALYTAGISAGDFIERNLLTVVATHSEGLPITYTLIQESMEADPTLEAVQESAFILNPETGVLSLNFQPTAAMHGMFEFEVEATDSRRETARTEVKVYVISDRNRVFFTFNNPLPEVTPQEDFIAETFTAFFGMTCNIDQTWWASDPVTGATRDDQTEVRAHFIRDDLPVPAEEIEQLRGNPTLVNSIQRALEEQNLQLADLFTGETPILGGDAQARALYALAAVAAALALIVVVLLIVFFVRTRTLNRRLQALSMTKYSSQDSGLNRVGLAAPGTNKHAVEGSNPIWNETLKAPDFDALSEQSYDSDLIGIEDLPQFRNDYFPPEEGSSMRGVVNEHVPESIANHNNNFGFNSTPFSPEFANTQFGR

SEQ ID NO: 5 - DNA, 5371 bp, Diabrotica virgifera
agagcatatcgaaagataataaagttaatcacaggtataaaacttttttcatgaaatctgtaaaatatttagcactaaactatttgtttttgtgagagttacaagtgagagggtgttttgtaaatgatgtggattgtttgatacccttaaaaaacatcaaaaatggctacgagaaatctatgtttatgtatgcttatttggatgccactctttgagggcatagttggcgacgtcgcctttggcattgcacaaattcctggtggtaaggcaacagtagaagatttgaaaaaagggaaatacttacttaacatggaagaaaataatcatggcggcgtaataccaactccactttttactattacaggggtggacaacactgattgtcctaacttgaatgttgaatttactcaaggcatgaagtttaagttttcgattaatgaaagttgtatattctatgctgaacagacatttgactacgaatcgaatgaaagatcatacaatttcagaatttcaaagtccgtaaatgacgagatagatattgcattcagtatcaaaaatatcgatgatgaacctccacaattaggaacgtttaaatgcaactttaatgaacaattagactatagtcttgatgacacaccatgcaatactacattgcatgatcccgacggatggctggcacaagagaaaattttaatattcatcgacactaaggatgaagatatattcgcaattgatttgcgaaagcctctaccaaatgatactgggacagacacctacgtttttatgtatctcttaaagcaacttaattatgaggataccaatttttatcagtttacagtgcaagcaaatgattctggaggtaatctttcaccacaagaaagtgctgtggtaaatgttatcaacattagaagtagacctccaaaatggtcaaaaataacattatttgatcaatttgacgaactgaccgaacaagattacgatatacaagctttagatggagatactggaattcatgcagatatttgttatgcaaaactaggtgaagatttaccagacaattacataaacgtcagtaccgataaaaccaataaaaaaggccatattcacgttaatcccattgatagagataaggatgatctaacactataccattttaacatttctgcctatgtttgtgatgccccagactattttactgtaaatacagtgcagtattacatcatcgatattgacaataatcctcccaggatagttgaaaatgttggggatgacggaaagaacacaacatttgacgatgataacgaccataaaaatgttacactaagttatttggaaaattattcgagatcgtataattttagcacaaccattacagatagagatacgggcgaaaacgcgcaattcactgtaagtctcgaaaatgttgagggttctacggttgattatacacaaccctatctaatagtaccggacaatgcttacaagacaggatcattcataataagcgtgaaaaataagaccttcctggatttcgagaacgacacttggaaaaagcattcctattatgttgtttccaatggaaaaaaagacaaatcaaagactgacagaatgttaatatcagtctcactagaagactataacgatgaacttcctattttcgagaaagaatcatacacaacagaaataaatgagactgttgcgaacggcactcaaatactatacacccatgcaacagacagagatgcagaagatttcgaactgaaaatgaatatagttggaacttatgctgagaataggctaagtattgataaagatggaaatattaaagtagaggtagtcaatgccttcgattacgatgtcttaaattctgtgtatttccaagtaaccgcaacagataaggttaatcatgtgactagagtaccagtaacgattaacattttggatgttaataatgaagctcccgtaataaatcaagtggaccatatacaaatcgaagaaaaccaacaaaacggtgtagtgctaaatgtaacaataacagctacggatgtagataccacggctaatctgactgctactataaattgggaagattccaaagttaccaaaactaatggtgctgtagtaaaaactgacgctgtaataaaagctatgcaatttttagagatagaaaatacacaaacagatgacggtctggaaatgaaactaaaggtaataaataataatgacgataatccagacaagccagattttgaaactttcgatacattgtatttaagtatagttgttgaagaccgtaatactgatcctgattttgaacaaaatcgatatacagaaggtcaaattgtcatcaatatccttgatgttaacgataatccaccatattttccaccttctaatgatgacactcgacaggtccaggaaatgtctctgaaaggagtatcggttggctccataaaagctgtcgatgttgatttgaattcagaaattacctaccattgcacgcccgaatatgaaaagtttgactgggtcgatgtaaatcttacaactggcgccataactgtaaagaacgataaacaagttgacgcagacacagacaaaacgtattatttcaactatacttgttgggctcatgatggtgtgttcttttccaaaccgttagacatctcaatctatgtcatcgacacaaacaacgaagtgcctgtaatagattttccaaatgaagtacacgtaaaggaaaaatcacttatagatacagtaattaaaaagattgttacaagtgatttagatagagatgagccgtttcgtactgtaaactgtaattttgcaagcgatactgatccagactgtcaaatagaattctatattgataccaatgttcttaaagtgaaaagaaataaaactcttgatcgtgataagggaagaaaaacatatccttgtctatttgagtgcttggataatcctttaaatgttcgatcccaaggacaaaataaagccaataaaagctttactataatattggatgatataaatgatcatgcacctgtgcttatgacgaaggacctacaatgttctgaaaatttgaataaggacggcgaagtaggagagggcataataggagaagatattgatgatggtgataatgctaaaatagatttttctgtgttaagtatcgttgataaggaaaccaaaaatgacattcaagaatcttttaatatttctaaaattgatagtgattatgtattgaacgatacattgaagaaagtgcatcttatagccttcgaagatctcaaaggaaaatatggaacgtatgaagtcactttacacatgcatgacgaaggagatccaatgcagacaacagatccggatccaactttaacacttacaattgaaaaatggaattaccaaacaccaagtataatatttcctgaaaacgatcaaacatacattgtcctatcggatcaacaacctggtcaaccattggcactgtttaacaacactggaacttcaaatactttgcccgattttagtgcaacagatggtgaaacgaaggactattctaagtgggatgtgaaattttcttatacgcagactaattatgaagatgataaaattttcgtgatagaccatatacagccgtgcgtttctcaactacaagtcagcaaacatttcaactcggatctagtacgaagtaaaaagtataagctaactattacagccagtgttaaggatggagcagaacaggaaggtgaagcaggttactcgacatcagcaaatataagtatagttttcctcaacaacgacgctcagccgatctttcaaaatagtgactggagtgtatcatttgttgaattcaatacaacacaacctgcgaaacctttggaagaacaggctgagtatgagaacacgaaaggaggacttcctatctattaccatttctattccgaaaaccagaccctttccaaatattttgaggttgatgagacgtccggtgatttatcggttataggaaatcttacctatgattatgatcaagatatatcgtttcacatagttgcttcgaatgactcacaagtcagaatgttggatccacgatccagtctaaatgttactgttaattttcttccacgtaaccgcagagctcctcagtggaaaagtaccaaattctttggagcagttatgcctacattcgtaactgggaacctcatagtcaccgctcaagctcatgacgacgattatatcgatcaacaacgcggattaacgtgttctatatctagtgagattaaccgaattggggaaggattagataaaataataggcgaaccattttatttgagcactgaaaatgatgccgccaaaatatttttggactttacagtgcagactacaatgaccggtcgatttgaatttaaaatcaaagtagaggacaatagagatgactacggcaatggtccatttgaaagtgaagctgatacaacaatatttattattactaaagacaacaccgttgattttcaattttacaacgatattgaggatgttcaggacagagaaacaccgatgttaaaaataatatccgatatagtgggatatgatgcatatcgtcaaaatatagatacagtaacaaatagcggtctcgttaggaccagagcaaggctttatttcattgacagtaaaagcagccgtcggttgcaactcactaaaagtcccgcagacagtggattcgaactggttaatagtgaaacaatattaaacatagtgaccaatgtgaacaccttccaaaatctggccagcaccttgagaagtgaacaaaaactgaacctcgacagctttgaaacgaattccaaatccggcaacagcgaagcagcactaagagcttggttgatcggagtgtccgtggtcttggggatcttggtcttgcttttactaatcactttaatactaaaaaccagacagttaagtaacagaattaaaaaactaactaccccccaatttggttctcaagagtctggacttaacaggatgggcataaatgcacccaccaccaacaagcatgccatcgaaggaacaaatccagtatacaataataacgaaatcaagaagccaaaaaatatgaacgattttgatactcacagcataagaagcggtgattctgatttggttggaatagaaaacaacccagagtttgactacaacttcaacactaacgaggataaaactacatatctctaaactatttttataataatcttatgtctagcttaagttggtcttaatagatattaatgtaatctgatttaactataattgtattataagtattaaatatgtataaaataaacacttattagatccaaaaaaaaaaaaaaaaaaa

SEQ ID NO: 6 - protein, 1688 aa, Diabrotica virgifera
MATRNLCLCMLIWMPLFEGIVGDVAFGIAQIPGGKATVEDLKKGKYLLNMEENNHGGVIPTPLFTITGVDNTDCPNLNVEFTQGMKFKFSINESCIFYAEQTFDYESNERSYNFRISKSVNDEIDIAFSIKNIDDEPPQLGTFKCNFNEQLDYSLDDTPCNTTLHDPDGWLAQEKILIFIDTKDEDIFAIDLRKPLPNDTGTDTYVFMYLLKQLNYEDTNFYQFTVQANDSGGNLSPQESAVVNVINIRSRPPKWSKITLFDQFDELTEQDYDIQALDGDTGIHADICYAKLGEDLPDNYINVSTDKTNKKGHIHVNPIDRDKDDLTLYHFNISAYVCDAPDYFTVNTVQYYIIDIDNNPPRIVENVGDDGKNTTFDDDNDHKNVTLSYLENYSRSYNFSTTITDRDTGENAQFTVSLENVEGSTVDYTQPYLIVPDNAYKTGSFIISVKNKTFLDFENDTWKKHSYYVVSNGKKDKSKTDRMLISVSLEDYNDELPIFEKESYTTEINETVANGTQILYTHATDRDAEDFELKMNIVGTYAENRLSIDKDGNIKVEVVNAFDYDVLNSVYFQVTATDKVNHVTRVPVTINILDVNNEAPVINQVDHIQIEENQQNGVVLNVTITATDVDTTANLTATINWEDSKVTKTNGAVVKTDAVIKAMQFLEIENTQTDDGLEMKLKVINNNDDNPDKPDFETFDTLYLSIVVEDRNTDPDFEQNRYTEGQIVINILDVNDNPPYFPPSNDDTRQVQEMSLKGVSVGSIKAVDVDLNSEITYHCTPEYEKFDWVDVNLTTGAITVKNDKQVDADTDKTYYFNYTCWAHDGVFFSKPLDISIYVIDTNNEVPVIDFPNEVHVKEKSLIDTVIKKIVTSDLDRDEPFRTVNCNFASDTDPDCQIEFYIDTNVLKVKRNKTLDRDKGRKTYPCLFECLDNPLNVRSQGQNKANKSFTIILDDINDHAPVLMTKDLQCSENLNKDGEVGEGIIGEDIDDGDNAKIDFSVLSIVDKETKNDIQESFNISKIDSDYVLNDTLKKVHLIAFEDLKGKYGTYEVTLHMHDEGDPMQTTDPDPTLTLTIEKWNYQTPSIIFPENDQTYIVLSDQQPGQPLALFNNTGTSNTLPDFSATDGETKDYSKWDVKFSYTQTNYEDDKIFVIDHIQPCVSQLQVSKHFNSDLVRSKKYKLTITASVKDGAEQEGEAGYSTSANISIVFLNNDAQPIFQNSDWSVSFVEFNTTQPAKPLEEQAEYENTKGGLPIYYHFYSENQTLSKYFEVDETSGDLSVIGNLTYDYDQDISFHIVASNDSQVRMLDPRSSLNVTVNFLPRNRRAPQWKSTKFFGAVMPTFVTGNLIVTAQAHDDDYIDQQRGLTCSISSEINRIGEGLDKIIGEPFYLSTENDAAKIFLDFTVQTTMTGRFEFKIKVEDNRDDYGNGPFESEADTTIFIITKDNTVDFQFYNDIEDVQDRETPMLKIISDIVGYDAYRQNIDTVTNSGLVRTRARLYFIDSKSSRRLQLTKSPADSGFELVNSETILNIVTNVNTFQNLASTLRSEQKLNLDSFETNSKSGNSEAALRAWLIGVSVVLGILVLLLLITLILKTRQLSNRIKKLTTPQFGSQESGLNRMGINAPTTNKHAIEGTNPVYNNNEIKKPKNMNDFDTHSIRSGDSDLVGIENNPEFDYNFNTNEDKTTYL

SEQ ID NO: 7 - DNA, 5277 bp, Heliothis virescens
atggcagtcgacgtgagaatagtaacggcagcggtattgattctcgctgctaatttaactttcgcgcaagattgttcctatatggtagcaattcccagaccagagcgacctgactttcctaatcaaaatttcgaaggagtaccatggagtcagaaccccctgttaccagcggaggatagggaagatgtgtgcatgaacgcgtttgatccaagtgccttgaaccccgtcaccgtcatcttcatggaggaggagatcgaaggggacgtggccattgccaggcttaactaccgaggtaccaatactccgaccgtggtaactccatttaactttggtaccttccacttgttggggccggtcatacgtaggatccccgagcaagggggggactggcatcttgttattacgcagaggcaggactatgagaccccgaacatgcagcagtatatcttcaacgtgagagtagaggatgagccccaggaagccactgtgatgctcatcattgtcaacatcgacgacaacgctcctatcatacagatgttcgagccttgtgacattcctgaacacggcgaaaccggcaccacagaatgcaagtacgtagtgagcgatgctgacggcgagatcagcacacgtttcatgacgtttgaaatcgagagcgatcgaaacgacgaagaatatttcgaactcgtgagagagaatattcagggacagtggatgtacgtccatatgaggcttatactcaacaaacctcttgactatgaggaaaacccgctgcatttgtttagagttacagctttggattccctaccaaacgttcatacagtgacgatgatggtgcaagtcgagaacatagagagcagaccaccgcggtggatggagaccttcgccgtgcagcagttcgatgagaagacagcacaagccttcagggttcgagccatcgatggagacacgggaatcgataaacctattttctataggattgaaactgaagaaagcgagaaagatttgttcagtgttgaaacaataggagctggtcgagaaggtgcttggtttaaagtcgctccaatagacagagacactcttgaaaaggaagttttccacgtgtctctaatagcgtacaaatatggcgacaatgacgtggaaggaagttcgtcattcgagtcgaaaaccgatatcgtcattattgtgaacgacgtgaatgatcaggcgccggtgcctttccgtccttcatacttcattgaaattatggaggaaactgcgatgacattgaatttagaggactttggtttccacgatagagatcttggtccgcacgcgcagtacacggtacacctggagagcatctccccggcgggagcgcacgaggcgttctacatcgcgccggaggtgggctaccagcgacagtccttcatcgtcggcacgcagaaccatcacatgctggacttcgaagtgccagagttccagaagatacaacttagggcagtagccatagacatggacgatcccaggtgggttggtatcgcgattataaacattaacctgatcaactggaacgatgagctgccgatcttcgagcacgatgtgcagactgtgaccttcaaggagacggagggcgctggcttccgggtcgccactgttctggcaaaggacagggatattgatgatagagtcgaacattctctaatgggcaacgcagtgaattacctgagtatcgacaaagacaccggtgacatcctcgtgacaattgacgatgcattcaactatcacagacagaacgagctctttgtgcagatacgagctgacgacacgttgggagagccgtataatacgaacactgcccaactggtgatacagctgcaagacatcaataacacacctccaacgctcagactgccccgcacgactccgtcagtggaagagaacgtgccggacgggttcgtgatccccaccgagctgcacgcctccgaccccgacaccaccgccgagctgcgcttcagcatcgactgggacacttcctatgccaccaagcagggcagggatgctgatgctaaggagtttgttaattgcatagaaatcgagacggtatacccgaacttgaacgaccgaggcaccgccatcggccgcgtggtggttcgcgagatccgggaacacgtcactatagactacgagatgttcgaggtgctgtacctcaccgtcagggtcacggatctcaacacggtcattggagacgactatgatatatcaacattcacgatcataataatagacatgaacgacaaccctccgctgtgggtggaaggcacgctgacgcaggagttccgcgtgcgagaggtcgccgcctcaggagttgttataggatccgtactcgccactgatattgatggacctctttataatcaagtgcggtataccatcactcctagattagacactccagaagacctagtggagatcgacttcaattcgggtcagatctcagtgaagaagcaccaggctatcgacgcggacgagccgccgcgccagcacctctactgcaccgtggtcgccagcgacaagtgcgacctgctctctgtcgacgtctgtccgcctgaccctaactacttcaacacaccgggtgaaataacgatccacataacagacacgaacaacaaggtgcctcgagtggaggaggacaagttcgacgaaaccgtctatatctacgagggcgcggaggacggagaacaagtcgtgcagctcttcgccagcgatctggatagagatgaaatctaccacaaagtgagctaccagaccaactacgcgatcaaccctcgtctccgcgacttcttcgaggtagacctggagaccggtctggtgtacgtcaacaacacggccggggagaagctcgaccgggacggcgatgaacccacgcatcggatcttcttcaacgtcatcgataacttctatggggaaggagacggcaaccggaaccaggacgagacccaagtgttagtggtgctgttggacatcaacgacaactatccggaactgcctgagggtctctcatgggatatctctgagagcttgctacagggtgtccgtgtaaccccagatatcttcgccccggaccgcgacgagcccggcaccgacaactcccgcgtggcgtacgacatcgtcagcctcacgcccaccgacagggacatcacacttcctcaactcttcaccatgatcaccatagagaaggacaggggcatcgaccagactggagaactggagaccgctatggatttaagaggctattggggcacttatgaaatacatgtcaaggcatacgaccatggagtacctcaaaggatttcctacgagaagtacccgctagttataagaccttacaacttccacgatcctgtgtttgtgttccctcaacctggaatgactatcagactcgcgaaggagcgagcagtagtgaacggcgtgctggcgacagtggacggcgagttcctggagcgaatcgtcgctaccgacgaggacggcttacacgctggagttgtcaccttctctatctcgggagatgatgaggcgttgcagtacttcgacgtgtttaacgacggagtgaacttaggtgcgctgaccatcacgcagctcttccctgaagacttccgagagtttcaggtgacgattcgtgctacggatggtggtacggagcctggtccaaggagtacggactgcaccgtcaccgtagtgtttgttcctacgcagggagagcctgtgttcgagacaagcacctacacggtcgcttttattgagaaagatgctggtatggaggaacgggctacgctgcctctcgccaaggacccgcggaacataatgtgtgaagatgattgtcacgacacctattacagcattgttggaggcaactcgatgggccactttgcagtggacccccagtccaacgagctgttcctgctgacaccgctggaccgcgcggagcaggagacgcacaccctcatcatcggcgccagcgactcgcccagcccggccgccgtgctgcaggcttccaccctcactgttactgtcaatgttcgagaagcaaatccgcggccagtgttccagagcgctctgtacacagccggcatctccaccctcgacaccatcaacagagctctgctgacactacacgcgacacattcagaaggcctgcccgtgacctacacgctgatacaagactccatggaagctgactccacactgcaagctgtgcaggagacagccttcaacctcaaccctcagactggagtgctgaccctcaacttccagccaactgcctccatgcacggcatgtttgagttcgatgtgatggctattgatacagtgggagaaaccgcacgcaccgaagtgaaggtgtacctgatatccgaccgcaacagagtgttcttcacgttcatgaacacgctcgaagaagtcgaaccgaatgaggatttcatggcggagacatttaccctgttcttcggcatgcggtgcaacatcgaccagacgctgcccgccagtgaccccgccaccggcgccgccagggacgaccagaccgaagtcagggcacacttcatacgcgacgacctgcctgtgccggctgaggagatcgaacagttgcgcggtaatccaaccctagtggcgacaatccagaacgccctgcaggaggagaacctgaacctggccgacctgttcacgggcgagactcccatcctgggcggcgaggcgcaggcgcgggcggtgtacgcgctggcggcggtggcggctgcgctcgcgctgctctgtgtcgtactgcttatactcttcttcatcaggactagggccctcaaccgtcgcctggaagctctctccatgaccaagtatagttcccaagactcaggactaaaccgcgtgggtctggcggcgccgggcaccaacaagcacgcggtggagggctccaaccccatctggaacgaaaccctcaaggcaccggactttgatgctcttagcgagcagtcgtacgactcggacctaatcggcattgaagacttgccgcagttcaggaacgactacttcccgcctgacgaggagagctccatgcggggagtcgtcaatgaacacatgcctggagctaattcagtagcaaaccataacaataacttcgggttcaacgctaccccctttagcccagagttcgcgaactcgcagctcagaagataaaatattatagtattttttatacaatattatatagaagtgatataacgcactaaaatttacctataagtatgggcgaag

SEQ ID NO: 8 - protein, 1732 aa, Heliothis virescens
MAVDVRIVTAAVLILAANLTFAQDCSYMVAIPRPERPDFPNQNFEGVPWSQNPLLPAEDREDVCMNAFDPSALNPVTVIFMEEEIEGDVAIARLNYRGTNTPTVVTPFNFGTFHLLGPVIRRIPEQGGDWHLVITQRQDYETPNMQQYIFNVRVEDEPQEATVMLIIVNIDDNAPIIQMFEPCDIPEHGETGTTECKYVVSDADGEISTRFMTFEIESDRNDEEYFELVRENIQGQWMYVHMRLILNKPLDYEENPLHLFRVTALDSLPNVHTVTMMVQVENIESRPPRWMETFAVQQFDEKTAQAFRVRAIDGDTGIDKPIFYRIETEESEKDLFSVETIGAGREGAWFKVAPIDRDTLEKEVFHVSLIAYKYGDNDVEGSSSFESKTDIVIIVNDVNDQAPVPFRPSYFIEIMEETAMTLNLEDFGFHDRDLGPHAQYTVHLESISPAGAHEAFYIAPEVGYQRQSFIVGTQNHHMLDFEVPEFQKIQLRAVAIDMDDPRWVGIAIININLINWNDELPIFEHDVQTVTFKETEGAGFRVATVLAKDRDIDDRVEHSLMGNAVNYLSIDKDTGDILVTIDDAFNYHRQNELFVQIRADDTLGEPYNTNTAQLVIQLQDINNTPPTLRLPRTTPSVEENVPDGFVIPTELHASDPDTTAELRFSIDWDTSYATKQGRDADAKEFVNCIEIETVYPNLNDRGTAIGRVVVREIREHVTIDYEMFEVLYLTVRVTDLNTVIGDDYDISTFTIIIIDMNDNPPLWVEGTLTQEFRVREVAASGVVIGSVLATDIDGPLYNQVRYTITPRLDTPEDLVEIDFNSGQISVKKHQAIDADEPPRQHLYCTVVASDKCDLLSVDVCPPDPNYFNTPGEITIHITDTNNKVPRVEEDKFDETVYIYEGAEDGEQVVQLFASDLDRDEIYHKVSYQTNYAINPRLRDFFEVDLETGLVYVNNTAGEKLDRDGDEPTHRIFFNVIDNFYGEGDGNRNQDETQVLVVLLDINDNYPELPEGLSWDISESLLQGVRVTPDIFAPDRDEPGTDNSRVAYDIVSLTPTDRDITLPQLFTMITIEKDRGIDQTGELETAMDLRGYWGTYEIHVKAYDHGVPQRISYEKYPLVIRPYNFHDPVFVFPQPGMTIRLAKERAVVNGVLATVDGEFLERIVATDEDGLHAGVVTFSISGDDEALQYFDVFNDGVNLGALTITQLFPEDFREFQVTIRATDGGTEPGPRSTDCTVTVVFVPTQGEPVFETSTYTVAFIEKDAGMEERATLPLAKDPRNIMCEDDCHDTYYSIVGGNSMGHFAVDPQSNELFLLTPLDRAEQETHTLIIGASDSPSPAAVLQASTLTVTVNVREANPRPVFQSALYTAGISTLDTINRALLTLHATHSEGLPVTYTLIQDSMEADSTLQAVQETAFNLNPQTGVLTLNFQPTASMHGMFEFDVMAIDTVGETARTEVKVYLISDRNRVFFTFMNTLEEVEPNEDFMAETFTLFFGMRCNIDQTLPASDPATGAARDDQTEVRAHFIRDDLPVPAEEIEQLRGNPTLVATIQNALQEENLNLADLFTGETPILGGEAQARAVYALAAVAAALALLCVVLLILFFIRTRALNRRLEALSMTKYSSQDSGLNRVGLAAPGTNKHAVEGSNPIWNETLKAPDFDALSEQSYDSDLIGIEDLPQFRNDYFPPDEESSMRGVVNEHMPGANSVANHNNNFGFNATPFSPEFANSQLRR

SEQ ID NO: 9 - DNA, 4587 bp, Helicoverpa armigera
atggcgacgaaacccaagactcctgggttcacaggtttgggagatgacagcgaagacgagtcggagtacactccgctttatgacgacgtcgacgatttagaacaaagaacagctcaagaaacaaaaggatggaatttgttccgagaactccccgtgaagaaggagagcgggtccatggcgtccacagcatggatcgacaccagtgtcaagatcctcaagtttctggcatacatcaccatatttgtcgtcgtactcggatctgctgttatttctaaaggcactctcctttttatcacttctcaacttaagaagggcaaacatatcactcattgtaacagggcattagctttagatcaacagtttataacagtccactctttggaggagcgcgtgacatggctatgggcagccttcataatgttcagtttcccggaggtgggcgtattcttaagatctgtcaggatatgcttcttcaaaaccgctctgaagcctacattccttcactttcttgcgtctatagtaatagaaaccctgcacactgttggaattgcgatgctcgttctgatcattctgcccgaactagatgtcgttaaaggcacaatgttgatgaatgcgatgtgcttcgtgccagggctcctgaacgccctctcgagagacmgaaatgagcgccgatatgtttggaagatcatgttagacgtactggcgatctccggccaagctactgccttcgtagtctggcctctccttaaaggcgatactattctatggactattccggtagcttgcgtgtttgtttcactcggctggtgggaaaacttcgtcggcagctcggatcaacaatggtcagtcctccgacctcttcaagarcttcgagatggtttaaaaagractcgttattacacgcagagagttgtgtcwgtatggaagatatttatattcatgtgctgcattttgatatctttggaaatacaacatgatgayccttttgctttcttcacaaaactcaccactggttttgccgaccgcttctacatcgtacatgaggttcaagcagttcgagatgagttcgaaggcttcttgggctacgcagtgaagggattaacgttagaaataccagcttcatggtctaccccactatgggtggtcctcatccaagtgttggccgcatacgtttgtttcggagccagtaaattcgcctgtaaaatcctcattcagaacttcagctttacatttgcactgagcctcgtaggaccrgtcactattaacttcttgattgccgcatgcgggatgaggaatgcgaacccttgtgctttttaccgcactatacctgattacttgttcttcgatattccaccggtgtacttcctaaacgagttcgtagtacgcgagatgtcrtgggtwtggttgctgtggatagtctcccaagcttgggtgactgctcacacktggcagccgcggtgtgagcgactcgckgcyactgacaaactgttcgccaaaccttggtactgcagcgcggtgatagaccagtcgctgttgttgaataggaccaaggatgacgacactgatatagcgttagaggacctcaaaggtttggatgcagatgctgattctatagttagcggggaaaaagtttcaaaggatgtcaagccmtctgacagtataacaaggatctacgtrtgtgcaactatgtggcacgaaacgaaagaagaaatgatggagttcttgaaatctattttccgtctcgacgaagatcagagcgctcgaagggttgcacagaagtacttgggcattgtcgatcctgattactatgaactagaagtgcacattttcatggatgacgcgtttgaggtgtccgatcatagttcggaagattcgcaagtgaatcgtttcgtcacgtgcctcgtagacactatygaygaggctgcytcagargtccayctcacaaacgtragattaagaccccctaagaagtaccccaccccatacggcggccgactygtatggacactsccaggaaagaacaaaatgatttgccatctmaaagacaagtccaaaatcagacacaggaaaagatggtctcaggtgatgtacatgtactatttccttggtcaycgtttgatggacttgccratctctgtggatcgcaaggaggttatcgctgagaayacttatttgttggctytggatggygacattgacttyaagccgatagccgtcaccttgctgattgatctcatgaagaaggayaagaacttrggagcagcgtgyggacgtatccatcctgtgggctctggcttcatggcttggtatcaaatgttcgagtacgctattggtcattggctgcaaaaggcgacggaacacatgatcggctgcgtactctgtagtcctggatgcttctctctgttcagaggaaaggctttgatggacgacaacgtcatgaagaaatacactttgacttctcacgaagcccgacattacgtacaatatgatcaaggtgaggaccgttggctgtgcacattactactacaacgcggctaccgagtcgagtactctgccgcgtccgatgcgtacacgcactgtccggaacaattcgacgagttcttcaaccagcgacgacgatgggtaccctctactatggccaacatattcgatctgctggcrgatgctaarcgraccatctcgataaatgataatatttccacgctttatattatgtatcagtctatgcttatgttcggtacaatcctcggccccggcactatattcctgatgatggtgggagcgatgaacgccatcactcagatgagcatgtccaacgcgctcatactcaacttggtgcccattctcatattcatcgtagtctgtatgacttgcaagtctgaaacgcagctattcctggcgagcttgataacatgcgcatacgcaatggtgatgatgttagtcatagtggggatagtccttcaaatcgtcgaggacggatggctggccccgtccagtttgttcacggccgtcatattcgggactttcttcgtgacggcrgcccttcatccvcaggagatcatatgtttgctgtatctaactgtgtactatgtgaccattccgagtatgtacatgttgctcattatatactcgctgtgcaatctcaacaacgtgtcgtggggaactagggaggtggtgcagaagaaaacggctaaggaaatggaacaagaacgcaaagaagcagaagaagctaagaagaagatggacgagaagagcatacagaagtggttcggcaagagtgatgagaccagcggctccttggagatgagtgtggctggtctgttcaagtgtatgtgctgcaccaatcctaaggaccacaaggaggatctgcatctgctgcagattgctacagccatcgagaagattgataagagattggaagcgctcggtgcacctcccgaagagactgagccgttgaatcgtcgccggtcttcagctgtattacgtcgacagtcgttagacccgctcgccagagtgccggagtacgaagagagcgatgtatccagcgacgtacctagggacgagcgtgacgatcttatcaacccgtactggatagaagacgtgaatcttcagaagggtgaagtagacttcctgaccacggcggagactgagttctggaaggatctgatcgatgtatacttgaggcccattgatgaaaacaaacaggaactggaacgtatcaaaacggacttgaagaatcttcgcgacaagagcgtgttcgcgttcgtaatgctgaactctttgttcgtgctgatgatcttcctgctgcaactcaaccaggatcagctgcacttcaagtggcccttcggacagtcagccagtatagagtacgatgatcagatgaatgtgttccacataacacaagactacctgaccctggagccgatcgggtcgctgttcatcatattcttcgggtccatcatcatcatccagttcaccgctatgctgttccatcgactcggcacgctcacgcatctgctgtccaatgttcagctcaactggtacttcactaagaagccggacgacatgtcagacaacgccctaatagagtctcgagcactagaaatagccaaagaccttcaacgcctgaacacagatgacctagaaaagcgtgacaacaaccaacacgtgagcagaaggaagaccatacataacttagagaaagggaaggatactaagcagagcgttgtgaatcttgacgccaacttcaagaggaggcttactatactgcaaaatggggatgctgaactgatctcccgcctaccatccctgggaggaaccacagcgacgcgacgagctactctacgtgctctcaaaaccagacgcgactccgtggtggccgagcgccgccgctcacagatgcaagcccgagactccaccacagacttcatgttcaactcgcccggcgcgttggaggatctgggcagtcgggcgtcggttggagcgtacgtgaatcgtggctacgagcctgcgctcgatagcgaagtggaggacacgccgcctcctccgagaaggtccaccgtgcgcttccaggaccattacgcgtga

SEQ ID NO: 10 - protein, 1528 aa, Helicoverpa armigera
MATKPKTPGFTGLGDDSEDESEYTPLYDDVDDLEQRTAQETKGWNLFRELPVKKESGSMASTAWIDTSVKILKFLAYITIFVVVLGSAVISKGTLLFITSQLKKGKHITHCNRALALDQQFITVHSLEERVTWLWAAFIMFSFPEVGVFLRSVRICFFKTALKPTFLHFLASIVIETLHTVGIAMLVLIILPELDVVKGTMLMNAMCFVPGLLNALSRDRNERRYVWKIMLDVLAISGQATAFVVWPLLKGDTILWTIPVACVFVSLGWWENFVGSSDQQWSVLRPLQELRDGLKRTRYYTQRVVSVWKIFIFMCCILISLEIQHDDPFAFFTKLTTGFADRFYIVHEVQAVRDEFEGFLGYAVKGLTLEIPASWSTPLWVVLIQVLAAYVCFGASKFACKILIQNFSFTFALSLVGPVTINFLIAACGMRNANPCAFYRTIPDYLFFDIPPVYFLNEFVVREMSWVWLLWIVSQAWVTAHTWQPRCERLAATDKLFAKPWYCSAVIDQSLLLNRTKDDDTDIALEDLKGLDADADSIVSGEKVSKDVKPSDSITRIYVCATMWHETKEEMMEFLKSIFRLDEDQSARRVAQKYLGIVDPDYYELEVHIFMDDAFEVSDHSSEDSQVNRFVTCLVDTIDEAASEVHLTNVRLRPPKKYPTPYGGRLVWTLPGKNKMICHLKDKSKIRHRKRWSQVMYMYYFLGHRLMDLPISVDRKEVIAENTYLLALDGDIDFKPIAVTLLIDLMKKDKNLGAACGRIHPVGSGFMAWYQMFEYAIGHWLQKATEHMIGCVLCSPGCFSLFRGKALMDDNVMKKYTLTSHEARHYVQYDQGEDRWLCTLLLQRGYRVEYSAASDAYTHCPEQFDEFFNQRRRWVPSTMANIFDLLADAKRTISINDNISTLYIMYQSMLMFGTILGPGTIFLMMVGAMNAITQMSMSNALILNLVPILIFIVVCMTCKSETQLFLASLITCAYAMVMMLVIVGIVLQIVEDGWLAPSSLFTAVIFGTFFVTAALHPQEIICLLYLTVYYVTIPSMYMLLIIYSLCNLNNVSWGTREVVQKKTAKEMEQERKEAEEAKKKMDEKSIQKWFGKSDETSGSLEMSVAGLFKCMCCTNPKDHKEDLHLLQIATAIEKIDKRLEALGAPPEETEPLNRRRSSAVLRRQSLDPLARVPEYEESDVSSDVPRDERDDLINPYWIEDVNLQKGEVDFLTTAETEFWKDLIDVYLRPIDENKQELERIKTDLKNLRDKSVFAFVMLNSLFVLMIFLLQLNQDQLHFKWPFGQSASIEYDDQMNVFHITQDYLTLEPIGSLFIIFFGSIIIIQFTAMLFHRLGTLTHLLSNVQLNWYFTKKPDDMSDNALIESRALEIAKDLQRLNTDDLEKRDNNQHVSRRKTIHNLEKGKDTKQSVVNLDANFKRRLTILQNGDAELISRLPSLGGTTATRRATLRALKTRRDSVVAERRRSQMQARDSTTDFMFNSPGALEDLGSRASVGAYVNRGYEPALDSEVEDTPPPPRRSTVRFQDHYA

SEQ ID NO: 11 - DNA, 4572 bp, Spodoptera frugiperda
atggcgagaccaagaccttatggttttagggctttagatgaggagagtgatgacaattcggagttgactccgttgcacgatgataatgatgacctaggacaaagaacagctcaagaggcaaaaggatggaatctgtttcgagagattccggtgaagaaggagagtgggtctatggcctcaactgccgggatagacttcagtgtaaagatccttaaagtcctggcgtatatttttatatttggcatagtgctcggatctgcggttgtgtctaagggtacgctgctttttatcacatcacaactgaaaaagggcaaagcaatcgttcactgtaatagacagttagaactggacaagcagtttataacaatccattcgttgcaagagcgtgtgacgtggctatgggcagccttcatagcattcagtattccagaagttggcgttttcttgagatcagtcagaatatgcttcttcaaaacagcaccgaagccttctgttttacagtttttgacggccttcgtagtagacacccttcatacaataggcattggattactggtgcttttcatcctgccagaattagacgtggttaaaggaacaatgctaatgaatgctatgtgcttcatgcctggaatactaaacgctgtgaccagagaccgcacggactctcgatacatgttgaaaatggcactagatgtactagctatctccgctcaagccaccgcgttcgtcgtctggcctctgctaaaaggcgttagtatgctctggacgattcctgtcgcatgcgtattcatctcactcggatggtgggaaaatttcgtcggcgatatcggaaaacaatggccagtcctggaacctgtacaagaacttcgtgacaatttaaagaagactcgttactacacacagagggtgttgtctttgtggaagatattcatattcatgtgttgcatcctgatatctttggcggcacaagatgacagcccgctttctttcttcacggagtttgctactggatttggtgagcgcttctacaaagttcatgaggttcgagcgatacaggacgaatttgaaggttttctgggctacaaaattatggacttatacttcgatcaaatgccagcggcatgggccaccccactgtgggtggtgctgatccaggtcctggcttctttagtctgttttatggcaagtttgtctgcctgcaagattctgatacaaaacttcagctttacatttgcgttgagtcttgttggacctgtcaccatcaacttgttgatttggctttgcggcgagaggaacgcagatccctgcgcatatagtaatacgataccagattatctgttcttcgacataccaccggtgtatttcctgaaggagtttgtggtgaaagagatgtcgtggatttggttgctgtggctggtgtcgcaggcgtgggtgacggcccacaactggcgctcccgggccgagcgtctcgccgccagcgacaagctcttcaacaggccttggtactgcagccccgtcctcgacgtctccatgctgttgaacagaaccaagaatgaagaagcggaaataacgatagaggatctaaaagaaacagagagtgagggtgggtctatgatgagcggatttgaagcaaagaaagacataaagccttctgacaacattacgaggatatatgtctgcgcgactatgtggcacgaaacgaaagaagaaatgatggacttcttgaagtctatcctgcgtttcgatgaggatcagagcgcgcgtcgcgtcgcacagaagtacttgggcattgtagatcctgattactatgaactcgaagtacacatcttcatggacgatgctttcgaagtgtcggaccacagcgcggacgactcgaaagtgaatcccttcgtgacgtgtctcgtggagactgtcgacgaggctgcttcagaggtccatctcaccaacgtgaggttgaggccaccgaagaaattccccacaccgtacggcggccgactggtctggactctcccaggaaagaacaaaatgatatgccacctcaaagacaagtccaaaatacgacacaggaaaagatggtctcaagtgatgtacatgtactacctattgggccaccgcctgatggacgtgccgatctcagtggaccgcaaggaagtcatcgcagggaacacctacttactggctttggacggcgacattgacttcaaaccgacagcagtcacgttactaatcgatttgatgaagaaggataagaatttaggagcagcgtgcgggcgcatccatcctgtgggctcaggcttcatggcatggtatcaaatgttcgagtacgctattggtcattggctgcaaaaggcgactgaacacatgattggctgtgtactctgtagccctggatgcttctccctcttcagaggaaaggctttgatggacgacaacgttatgaagaaatataccttaacttcccacgaggcacgacactatgtgcaatacgatcaaggcgaggaccgttggtgcacgctactgctgcagcgcgggtaccgcgtggagtacagcgcggtgtcggacgcgtacacgcactgccccgagcacttcgacgagttcttcaaccagcgccgccgctgggtgccctccacgctcgccaacatcttcgacctgctcggcagcgccaagctcaccgtcaagtccaacgacaacatctccaccctctatatagtctatcagttcatgttgatagtgggtacggtgttgggtcccggcacgatcttcctgatgatggggggagccatgaacgccatcattcagatcagcaacgcgtacgcgatgatgttgaacctcgtaccactcgtcatcttccttatagtctgtatgacttgtcagtcaaagacgcagctcttcctcgctaacctcataacatgcgcatacgcaatggtgatgatgatcgtgatagtggggatagttctgcagatagtggaggatggatggctggctccgtccagtatgttcacagctttaatattcggtacattcttcgtcaccgcggcactacacccgcaagagatcaaatgtttgttgttcatagcagtgtactatgtaaccatccctagtatgtacatgttgttgatcatatactccatctgtaatctcaacaacgtatcctggggtaccagggagacaccgcagaagaaaactgctaaggaaatggaaatggaacagaaggaagcagaagaagcgaagaaaaaaatggagagtcagggtttgaagaagttgtttgccaagggagaagagaagagtggttcgttagagttcagtgtggcgggcctgttgcgatgtatgtgctgcaccaatccagaggatcataaggacgatctcaacatgatgcagatctcacacgcgttggagaagataaataagagattggatcaactcgatgtccctcctgagccgacccaccagccctcgcatccgcacacacacgtggagacggtcggtgttcgtgattacgaagacagcgagatttccactgaaattcctaaggaagaacgagacgacctgattaacccctactggatcgaggacgtggaactccagaagggcgaggtagacttcctcaccaccgctgagaccaacttctggaaggatgtcatcgatgaatacttactgcctattgatgaggacaagcgtgaaattgaacgtataagaaaagatttgaagaacttgcgagataagatggtgtttgcgttcgtgatgttgaactctctgttcgtgctcgtcatcttcctgctgcagctcagccaggaccagctgcacttcaagtggccattcggacagaagtccagcatggagtacgataatgatatgaatatgttcatcataacccaagactacttaacgctggaacctatcggtttcgtgttcctcctgttcttcggctccatcatcatgatccagttcaccgccatgttgttccatcgcctggacacgctggcccatctgctgtccaccaccaagctggattggtatttcagtaagaagccggacgacctatcagacgatgcgctaatagactcttgggcgttgacaatagcgaaggatcttcaacgtctgaacaccgacgacttggataaacgaaataacaacgaacacgtgtccaggaggaagaccatatataacttggagaaagggaaggaaaccaaaccggctgttatcaacctcgatgccaacgccaagaggagattgactatcctgcagaatgaagactcagaattgatctcccgcctgccatctctgggacctaatttggcaactcgtcgtgccacggtgcgtgcaataaacactcgacgcgcatctgtcatggcggagcgacgcaggtctcagttccaagcgcgaccttccgggggatcatacatgtataataaccctcaaaacacgattcagctggacgatatggtcggggggccgtcgacgtcgggagtgtacgtgaaccgagggtacgagcccgccctggacagcgacatcgaggacacgcccgtgcccaccagacgatccgttgtacacttcaccgaccatttcgcgtga

SEQ ID NO: 12 - protein, 1523 aa, Spodoptera frugiperda
MARPRPYGFRALDEESDDNSELTPLHDDNDDLGQRTAQEAKGWNLFREIPVKKESGSMASTAGIDFSVKILKVLAYIFIFGIVLGSAVVSKGTLLFITSQLKKGKAIVHCNRQLELDKQFITIHSLQERVTWLWAAFIAFSIPEVGVFLRSVRICFFKTAPKPSVLQFLTAFVVDTLHTIGIGLLVLFILPELDVVKGTMLMNAMCFMPGILNAVTRDRTDSRYMLKMALDVLAISAQATAFVVWPLLKGVSMLWTIPVACVFISLGWWENFVGDIGKQWPVLEPVQELRDNLKKTRYYTQRVLSLWKIFIFMCCILISLAAQDDSPLSFFTEFATGFGERFYKVHEVRAIQDEFEGFLGYKIMDLYFDQMPAAWATPLWVVLIQVLASLVCFMASLSACKILIQNFSFTFALSLVGPVTINLLIWLCGERNADPCAYSNTIPDYLFFDIPPVYFLKEFVVKEMSWIWLLWLVSQAWVTAHNWRSRAERLAASDKLFNRPWYCSPVLDVSMLLNRTKNEEAEITIEDLKETESEGGSMMSGFEAKKDIKPSDNITRIYVCATMWHETKEEMMDFLKSILRFDEDQSARRVAQKYLGIVDPDYYELEVHIFMDDAFEVSDHSADDSKVNPFVTCLVETVDEAASEVHLTNVRLRPPKKFPTPYGGRLVWTLPGKNKMICHLKDKSKIRHRKRWSQVMYMYYLLGHRLMDVPISVDRKEVIAGNTYLLALDGDIDFKPTAVTLLIDLMKKDKNLGAACGRIHPVGSGFMAWYQMFEYAIGHWLQKATEHMIGCVLCSPGCFSLFRGKALMDDNVMKKYTLTSHEARHYVQYDQGEDRWCTLLLQRGYRVEYSAVSDAYTHCPEHFDEFFNQRRRWVPSTLANIFDLLGSAKLTVKSNDNISTLYIVYQFMLIVGTVLGPGTIFLMMGGAMNAIIQISNAYAMMLNLVPLVIFLIVCMTCQSKTQLFLANLITCAYAMVMMIVIVGIVLQIVEDGWLAPSSMFTALIFGTFFVTAALHPQEIKCLLFIAVYYVTIPSMYMLLIIYSICNLNNVSWGTRETPQKKTAKEMEMEQKEAEEAKKKMESQGLKKLFAKGEEKSGSLEFSVAGLLRCMCCTNPEDHKDDLNMMQISHALEKINKRLDQLDVPPEPTHQPSHPHTHVETVGVRDYEDSEISTEIPKEERDDLINPYWIEDVELQKGEVDFLTTAETNFWKDVIDEYLLPIDEDKREIERIRKDLKNLRDKMVFAFVMLNSLFVLVIFLLQLSQDQLHFKWPFGQKSSMEYDNDMNMFIITQDYLTLEPIGFVFLLFFGSIIMIQFTAMLFHRLDTLAHLLSTTKLDWYFSKKPDDLSDDALIDSWALTIAKDLQRLNTDDLDKRNNNEHVSRRKTIYNLEKGKETKPAVINLDANAKRRLTILQNEDSELISRLPSLGPNLATRRATVRAINTRRASVMAERRRSQFQARPSGGSYMYNNPQNTIQLDDMVGGPSTSGVYVNRGYEPALDSDIEDTPVPTRRSVVHFTDHFA

SEQ ID NO: 13 - DNA, 2862 bp, Helicoverpa armigera
atgggtgccaaaatgttgcttcccaccgtattctgcatcctcctgggatccatagcggccattcctcaagaggacttcaggtccaacttggagtggtctgactacagcaccaacttagacgagccggcgtaccgtctgcgtgatgtggtctatcctactgatgtcaacctggatctggatgtctacctaaaccacctgaacttctctggacttgtacagattgatgttcaagtacgagagaacaatttacgccaaattgttcttcaccaaaaggtggtttccatcactggagtgaatgttgtcggacctaacggcccagttcctctccagttcccccacccttataccactgatgattactatgagatccttctcatcaacttggaccagcccatcaacattggcaactactccatcgccatcagatacaatggccagatcaatgctaaccctcttgaccgaggtttttacagaggctactatcacctgaacaatgaattgagggtctacgccaccacccagttccagccttaccacgccaggaaggccttcccttgcttcgatgaaccccaattcaaatcccgctacactatctccattactcgcgacaccagtctgtctccatcgtactccaacatggctattagaagcgcccaagacattagtacctcccgtattcgcgaaaacttctacactacgcccatcatctccgcctatctggtcgctttccacgtcagtgacttcgtctccactgaatacaccagcaccgatgccaaacccttcagtattatctcccgccaaggtgccacgaaccagcaccaatatgctgctgaaatcggtcttaagatcaccaacgaactcgatgactactttggcatccagtaccatgagatgggacaaggtgctttgatgaagaatgaccacatcgctcttcctgacttcccctccggtgctatggagaactggggaatggttaactacagggaagcctacctcttgtacgacgaaaacaacaccaacttgaacaacaagattttcatcgctaccatcatggctcacgaattgggacacaaatggttcggtaacctcgtcacttgcttctggtggagcaacctttggcttaacgagtctttcgccagcttcttcgaatacttcggcgctcactgggctgatcccgctctagagttagatgaccagtttgtcgttgactacgtgcacagcgctctcaactctgacgccagccagtacgccactcccatgaaccacaccgacgtcgtggacaatgactccatcacctcccacttcagtgttaccagctacgctaagggagcttccgtccttaagatgatggaacatttcgttggatggaggaccttcagaaacgctctcagatactacttgagaaacaacgagtacgacatcggtttccccgttgatatgtacacggctttcaagcaagcagtcgctgaagattttactttccaacgtgatttccctaatgttgacgttggcgcagtattcgacagctgggtccagaaccctggctctcccgtcatcaacgttgcccgtaacaataacacaggtgtcatcactgtcaaccagcaacgttacgtgctctccggcgctgttgcctcaacgacgtggcacattcctctcacctggactcaacatggctccctcaacttcaacagcaccaggcctagcaccgtccttagcgatgagattggcaccatcaacgctgcatctggagaccacttcgtcattttcaacattgcccaatctggtctgtaccgtgtcaactacgacaccaacaactggcagttgcttgcttcatacctgaagagcaacaacagacagaacattcacaagctgaacagagctcagatcgtcaacgacatcttgtacttcgtgcgttctaacagcatcaacaggactctcgcttttgatgtcctcgacttcttgagggatgagaccgattactacgtatggaatggagctcttacccagatcgactggatccttcgtcgccttgaacacttgcctaccgctcatgctgctttctctgaatacatcctcgagctcatgaacacggttatcaatcaccttggctataacgagcacagtaccgactctacctccacaatcctcaaccgtatgcagatcatgaactacgcttgcaaccttggacacagtggctgcatttctgacagtttagacaaatggaggcagcaccgtgctaacgtatctaacttggtaccagtaaatctccgtcgttacgtttactgcgttggtcttcgtgagggtaacgaaactgactacaactacctgtatagcgtgtacaattcttcagagaacactgctgacatggttgttatcctccgcgccctcgcttgcaccaagcatcagccttctcttgagcactacttgcaacagtccatgtacaacgacaaagttcgtatccacgaccgcaccaacgcgttctccttcgctctgcaaggcaaccctgagaaccttcccatcgttctgaacttcctctacaacaactttgccgctatcagggaaacgtacggaggtgttgcccgtctcaatatttgcctcaacgctattgcggcattcttgactgactaccagaccatcactcagttccaaacttgggtgtactccaaccaattggagctggttggctctgtcggcgttggaaataacgtcgtcgccgccgccttgaacaatctcacttggggcaacggcgcagctgttgaaattgtcaacttcctcaactctagaagcggttccaccaccatccttgcttcttcaatcctcatcttagcagccatgcttctacaaatgttccgctaagatgtc

SEQ ID NO: 14 - protein, 951 aa, Helicoverpa armigera
MGAKMLLPTVFCILLGSIAAIPQEDFRSNLEWSDYSTNLDEPAYRLRDVVYPTDVNLDLDVYLNHLNFSGLVQIDVQVRENNLRQIVLHQKVVSITGVNVVGPNGPVPLQFPHPYTTDDYYEILLINLDQPINIGNYSIAIRYNGQINANPLDRGFYRGYYHLNNELRVYATTQFQPYHARKAFPCFDEPQFKSRYTISITRDTSLSPSYSNMAIRSAQDISTSRIRENFYTTPIISAYLVAFHVSDFVSTEYTSTDAKPFSIISRQGATNQHQYAAEIGLKITNELDDYFGIQYHEMGQGALMKNDHIALPDFPSGAMENWGMVNYREAYLLYDENNTNLNNKIFIATIMAHELGHKWFGNLVTCFWWSNLWLNESFASFFEYFGAHWADPALELDDQFVVDYVHSALNSDASQYATPMNHTDVVDNDSITSHFSVTSYAKGASVLKMMEHFVGWRTFRNALRYYLRNNEYDIGFPVDMYTAFKQAVAEDFTFQRDFPNVDVGAVFDSWVQNPGSPVINVARNNNTGVITVNQQRYVLSGAVASTTWHIPLTWTQHGSLNFNSTRPSTVLSDEIGTINAASGDHFVIFNIAQSGLYRVNYDTNNWQLLASYLKSNNRQNIHKLNRAQIVNDILYFVRSNSINRTLAFDVLDFLRDETDYYVWNGALTQIDWILRRLEHLPTAHAAFSEYILELMNTVINHLGYNEHSTDSTSTILNRMQIMNYACNLGHSGCISDSLDKWRQHRANVSNLVPVNLRRYVYCVGLREGNETDYNYLYSVYNSSENTADMVVILRALACTKHQPSLEHYLQQSMYNDKVRIHDRTNAFSFALQGNPENLPIVLNFLYNNFAAIRETYGGVARLNICLNAIAAFLTDYQTITQFQTWVYSNQLELVGSVGVGNNVVAAALNNLTWGNGAAVEIVNFLNSRSGSTTILASSILILAAMLLQMFR

SEQ ID NO: 15 - DNA, 3014 bp, Heliothis virescens
atgggtgccaaaatgttgcttcccaccgtgttttgtatccttctgggatccatagcagccattcctcaagaggacttcaggtcgaacttggagtgggctgactacagcaccaatttagacgagccggcgtaccgtctccgtgatgtggtgtaccctactgatgtcaacctggatctggacgtgtacctcgatgaactaaggttcaatggattggtacagattgatgtcgaagtacgagagaacgatttacgtcaaattgttcttcatcagaaggtcgtctcaatcaacgctgttaacgttgtcggacccaacggtccagtcggtctacagttcccatacccttacaccactgatgattactacgagatcctcctcatcaacctggctgagcccatcaacatcggcaactactccatcaccatcagatacaacggccagatcaatgataaccctatcgacagaggtttctacaaaggctactattacctgaacaatgaattgaggctctacgccaccacacagttccagccttaccacgccaggaaggccttcccgtgcttcgacgagccccaattcaaatcccgcttcactatctccatcactcgtgccagcagcctgtctccttcatactccaacatggctatcagcaacactcaaatccttggtgcccgtactcgcgaaactttccatcctacgcccatcatctccgcctacctggttgctttccacgtcagtgacttcgtcgccactgaatacaccagcaccgatgccaaacccttcagtattatctcccgccaaggtgtcacagaccagcacgaatatgccgctgagatcggtctcaagatcaccaacgagcttgatgactacttaggcatccagtaccatgagatgggacaaggtaccctgatgaagaatgaccacattgctcttcctgacttcccctccggtgctatggagaactggggaatggtaaactacagggaggcttaccttttgtacgacgctaacaacaccaacttaaacaataagattttcatcgctaccatcatggctcacgagctgggacacaaatggttcggtaacctcgtcacctgcttctggtggagcaacctttggctaaacgagtctttcgccagctttttcgaataccttggtgctcactgggctgatcccgctctagagttagatgaccagtttgtcgtcgactacgtacacagcgctctcaattctgacgccagtcaattcgccacccctatgaaccatgtcgacgttgtggacaacgactcaatcaccgcccacttcagtgtcactagctacgctaagggcgcttccgtccttaggatgatggaacacttcgttggatcgaggaccttcagaaatgccctcagatattacttgagaaacaacgagtatagtataggtttccccgttgatatgtacgcggctttcaagcaagccgtctctgaagattttaccttcgaacgtgatttccccggtattgacgttggagcagtattcgacacctgggtccagaaccgtggatctcccgtcttgaacgttgcccgtaacagcaacactggtgtcatcagtgtcagccaggaacgctatgtgctctcgggcgctgtagctccagcgttgtggcagattcctctcaccttgactcaaaatggctccctcaactttgagaacaccagacctagcttggtccttactacccagagccagaatatcaacggtgcctctggagataactttgttattttcaacaatgctcagtccggtctgtaccgtgttaactacgacacaaacaactggcagttgcttgcttcatacctgaagagcaacaacagagagaacattcacaagctgaacagagcccagatcgtcaacgatgtcttgaacttcgtgcgttccaacagcatcaacagaaccctcgcttttgaagttctcgacttcttgagagatgagaccgattactatgtatggaacggagctcttacccagatcgactggatccttcgtcgccttgagcatttgccggctgcccatgctgctttctctgaatacatccttgatctcatgagcacggtcatcaaccaccttggttacaatgagcagagtactgactccacctccacaatcctcaaccgaatgcagatcatgaactatgcctgcaatcttggacacagtggttgcattgctgacagtttagacaaatggaggcagcaccgtgagaacccgaataacttggtgccagtgaatctccgtcgttacgtgtactgcgtcggtctgcgtgaaggcaacgaaactgactacagctacttgttcagcgtgtacaattcctcagagaacaccgctgacatggttgtgatactccgcgctctcgcctgcaccaaacaccagccatctcttgagcactatctgcaagagtccatgtacaacgacaaaatccgtatccacgaccgtacaaatgcattctccttcgctctgcaaggcaaccctgagaaccttcccatcgtccttaacttcctctacaacaactttgccgctatcagggaaacgtatggaggtgtggcccgtctcaatctgtgcatcaacgcaatccctgcattcttgactgactaccagactattactcagttccaatcttgggtatacgcaaaccaattggcgttggttggttcattcaacaatggcgttagcgtcgtcaacaccgccttggataaccttacttggggcaacggtgctgctgttgagatcgtcaatttcctcaactacaagagtgcatccccctccatccttgcttcttccatcctcatcttagcagctatgctcgtacaaatgttccgctaaggtgtctcattctaagtcgttacacttcacataattctaatttaagtttatctattttgttttatacaatcgttccgtcgttttgtttatggttgtgaagtatttattgtagatttattgataataaatgttcttttgtaaagaaaaaaaaaaaaaaaaaa

SEQ ID NO: 16 - protein, 950 aa, Heliothis virescens
MGAKMLLPTVFCILLGSIAAIPQEDFRSNLEWADYSTNLDEPAYRLRDVVYPTDVNLDLDVYLDELRFNGLVQIDVEVRENDLRQIVLHQKVVSINAVNVVGPNGPVGLQFPYPYTTDDYYEILLINLAEPINIGNYSITIRYNGQINDNPIDRGFYKGYYYLNNELRLYATTQFQPYHARKAFPCFDEPQFKSRFTISITRASSLSPSYSNMAISNTQILGARTRETFHPTPIISAYLVAFHVSDFVATEYTSTDAKPFSIISRQGVTDQHEYAAEIGLKITNELDDYLGIQYHEMGQGTLMKNDHIALPDFPSGAMENWGMVNYREAYLLYDANNTNLNNKIFIATIMAHELGHKWFGNLVTCFWWSNLWLNESFASFFEYLGAHWADPALELDDQFVVDYVHSALNSDASQFATPMNHVDVVDNDSITAHFSVTSYAKGASVLRMMEHFVGSRTFRNALRYYLRNNEYSIGFPVDMYAAFKQAVSEDFTFERDFPGIDVGAVFDTWVQNRGSPVLNVARNSNTGVISVSQERYVLSGAVAPALWQIPLTLTQNGSLNFENTRPSLVLTTQSQNINGASGDNFVIFNNAQSGLYRVNYDTNNWQLLASYLKSNNRENIHKLNRAQIVNDVLNFVRSNSINRTLAFEVLDFLRDETDYYVWNGALTQIDWILRRLEHLPAAHAAFSEYILDLMSTVINHLGYNEQSTDSTSTILNRMQIMNYACNLGHSGCIADSLDKWRQHRENPNNLVPVNLRRYVYCVGLREGNETDYSYLFSVYNSSENTADMVVILRALACTKHQPSLEHYLQESMYNDKIRIHDRTNAFSFALQGNPENLPIVLNFLYNNFAAIRETYGGVARLNLCINAIPAFLTDYQTITQFQSWVYANQLALVGSFNNGVSVVNTALDNLTWGNGAAVEIVNFLNYKSASPSILASSILILAAMLVQMFR

SEQ ID NO: 17 - DNA, 2133 bp, Helicoverpa armigera
agcgactttactggttcagttagaatcgcgatggtgacactgttcccgtacgtagtggcggtgctgtgcggcgcgacgagcgcgcgcgcccactggctgcatcccgcggcgccggccgccgccagccgcgccgagacctctgccaactactgggcgcaagacgcgcaggccgccatcaacgctcgcctggaacgagttgaaagcgtgaagaaagcgcgtaacgtcatcatgttcctgggcgacggcatgtcggtgcccacgctcgccgccgcgcgcacgctgctcgggcagcgccaagggaaaacgggagaggagacaaagttgcatttcgagactttccccacaatcggattagtgaagacgtactgtgtggacgcccagattgcagactccgcatgtactgccacagcgtatttgtgtggtgtaaaaaataactatggcgccataggcgtagacggcacggtacgccgaggagactgtcaagccgcttcaaacactgcgacacacgtcgagtccatcgcggagtgggcgctcgctgacggacgagatgtcggtattgtgacgacgactcgtatcactcacgcgtctccggcgggcacgttcgcgaagacggcgaaccgcacctgggagaacgacggtgaagtgtcgcagatgggcttggacgccaaggactgccctgacatcgcgcatcagttggtacaccatcatcccggtaacaagttcaaggttatttttggtggtggcaagcgtgcctttttgccaaatactgaacaggacgaaaaaggatcttatggtagaaggatagacaaccgcaatctcatcaaagagtgggaggatgataaggtttctcgtaatgtcagccatcaatatgtttggcaccgcgagcagctaatgcgtctaaaggaggacctgcctgaatacatgttgggactgttcgagagcagtcatatgacctatcacttgaaatcagaccctcagtctgaacccactctcgctgaactaacagaggtggcaattcggtcattaagacgcaatgagaagggattcttcctgttcgtggagggggggcgcatcgaccacgcgcaccacgacaacctggtggagctcgcactcgacgagacgctggagatggacaaggccgtggccaccgccacgaagatgctctcagaggacgactcgctcatcgtggtcactgccgaccacgcacacgtcatgactttcaatggctactctaactgtggtcataacatcctcgggccctccagggatgtcggactagacaatgtgccttacatgacgctaacgtacgccaatggacccggattccgtccacacgttaacgacattagaccagatgttacccttgagccaaactatcgcaccctggactgggagtcgcacgtggacgtgccgctggtggacgagacgcacggcggcgacgacgtggccgtgttcgcgcgcgggccgcaccactccatgttcacggggctgtacgagcagagccagctgccgcacctcatggcctacgccgcctgcatcggccccggccggcacgcctgcgccagtgccgcgcacttgcctagcgcgcacttcttcgtagctctgctcgctctattcacttccattttactgcgataatatttattaattgaaatagctttaataaagtttcatacttaatagtcatcattacgttgacaagcagatctgtaatgtgttgaaataaaagtaaaagttatcatttaacatacgaaaaataaagttcacatagacatttgtaaacaatacaagattgatagacgttatttatttacgatttctgtacatacataggtacacataataaatacgaaaggaaagaaattaaatgtaatcgattttggagttgagctaattaataaccatgttgttaatattgcgagtttcattggccacgtgctgcatatctacattgtttgaacacatcttctaggtcttgaagcactgcagttgttgccatttattgcgtatcatgttgtttacaatacttaaaatattgaacctttttcattattaaacaatatgaactgtaaaaaaaaaaaaaaaaaaaaaaaaaaaaaaaaaaaaaaaaaaaaaaaaaaaaaaaaaaaaaaa

SEQ ID NO: 18 - protein, 535 aa, Helicoverpa armigera
MVTLFPYVVAVLCGATSARAHWLHPAAPAAASRAETSANYWAQDAQAAINARLERVESVKKARNVIMFLGDGMSVPTLAAARTLLGQRQGKTGEETKLHFETFPTIGLVKTYCVDAQIADSACTATAYLCGVKNNYGAIGVDGTVRRGDCQAASNTATHVESIAEWALADGRDVGIVTTTRITHASPAGTFAKTANRTWENDGEVSQMGLDAKDCPDIAHQLVHHHPGNKFKVIFGGGKRAFLPNTEQDEKGSYGRRIDNRNLIKEWEDDKVSRNVSHQYVWHREQLMRLKEDLPEYMLGLFESSHMTYHLKSDPQSEPTLAELTEVAIRSLRRNEKGFFLFVEGGRIDHAHHDNLVELALDETLEMDKAVATATKMLSEDDSLIVVTADHAHVMTFNGYSNCGHNILGPSRDVGLDNVPYMTLTYANGPGFRPHVNDIRPDVTLEPNYRTLDWESHVDVPLVDETHGGDDVAVFARGPHHSMFTGLYEQSQLPHLMAYAACIGPGRHACASAAHLPSAHFFVALLALFTSILLR

SEQ ID NO: 19 - DNA, 1680 bp, Heliothis virescens
cagttagtgcgatcgcgatgatgtcgctgtaccagtgcctactggccgtgctgtgctgtgcggcgtgcgcgcgcgcccactggttccaccccgcagcgacggcgggtcgcgcggcggccaccactcgcgtcgagacttctgccaactattgggtgcaagatgcgcaggcagccatcgacgctcgcctggcgcaagtggagagcgtgaagaaagcgcgtaacgtcatcatgttcctgggcgacggcatgtccgtgcccacgctggccgccgcgcgcacgctgctaggtcagcgccaagggaacacaggagaggagactaagttgcatttcgagacttttcccacaatcggactagtgaagacatactgtgtggacgctcagattgcagactccgcatgtactgccacggcgtatttgtgtggtgtaaagaataattatggtgctattggcgtagacgccacggtacggcgcggagactgccagacagcttcaaacactgccactcacgtcgagtccatcgcggagtgggcgctcgcggacggacgagatgtcggtatcgtgacgacgacgcgtattactcacgcgtctccagcgggcacattcgcgaagactgcgaaccgcacctgggagaacgacggagaagtatcgcaaatgggattgaacgctaaggactgccctgacatcgctcatcagttggtacaccaccatcccggtaacaagttcaaggttatttttggaggtggcaagcgcgcctttttgccaaacactgaacaggatgaaaaagggtcttacggtaggaggttagacaaccgcaaccttatcaaagagtgggagaatgataaagtgtctcgtaatgtgagtcatcaatatgtttggaatcgcgaacaactgatgagcctaaatgacgacctgccagagtacatgttgggcttgttcgagagtagtcatatgacatatcacatgaaatcagatcctcagtctgaacctactctcgctgaactaacagagttggcgattcggtcattgcggcgcaatgaaaagggattcttcctgttcgtggagggcggacgcatcgaccacgcgcaccacgacaacctggtggagctcgcgctcgatgagacgctggagatggacaaggccgtggcgaccgccacgcagctgctgtcggaggacgactcgctcattgtggtcaccgccgaccacgcacacgtcatgactttcaatggctactctaaccgtggccgtgacatcttggggccctccagggatgttgatctagacaacgtaccttacatgacgctaacgtatgccaatggacctggatttcgttcgcacgtgaacgacattcgaccagatgttacagctgagtcaaactaccgctctctggactgggagtcgcacgtggacgtgccgctggaggacgagacgcacggcggtgacgacgtggccgtgttcgcgcgcgggccgcgccactccatgttcacggggctgtacgagcagagccagctgccgcacctcatggcctacgccgcctgcatcggccccggccgacacgcctgcgtcagcgccgcgcacttgcccaccgcgcacttctttattgctctgtttgctctattcaccccgattttactaaaataataattatttagaatttacatcataaaaaaaaaaaaaaaaaaaa

SEQ ID NO: 20 - protein, 539 aa, Heliothis virescens
MMSLYQCLLAVLCCAACARAHWFHPAATAGRAAATTRVETSANYWVQDAQAAIDARLAQVESVKKARNVIMFLGDGMSVPTLAAARTLLGQRQGNTGEETKLHFETFPTIGLVKTYCVDAQIADSACTATAYLCGVKNNYGAIGVDATVRRGDCQTASNTATHVESIAEWALADGRDVGIVTTTRITHASPAGTFAKTANRTWENDGEVSQMGLNAKDCPDIAHQLVHHHPGNKFKVIFGGGKRAFLPNTEQDEKGSYGRRLDNRNLIKEWENDKVSRNVSHQYVWNREQLMSLNDDLPEYMLGLFESSHMTYHMKSDPQSEPTLAELTELAIRSLRRNEKGFFLFVEGGRIDHAHHDNLVELALDETLEMDKAVATATQLLSEDDSLIVVTADHAHVMTFNGYSNRGRDILGPSRDVDLDNVPYMTLTYANGPGFRSHVNDIRPDVTAESNYRSLDWESHVDVPLEDETHGGDDVAVFARGPHHSMFTGLYEQSQLPHLMAYAACIGPGRHACVSAAHLPTAHFFIALFALFTPILLK

SEQ ID NO: 21 - DNA, 1626 bp, Spodoptera frugiperda
  misc_feature (1182)..(1183): n is a, c, g, or t
atgaggtcgctactgacttacctagtggccgccgtggtggtggcggcgtgtgtccgcggggaccggtaccaccccgcggaccccggcagcagagctgacaccgttgcgaaccgtgccgagacctcagccaactactgggcccaagaagcgcaggctgcaatcaatgcccggctggcgcacaaggagagcgtgaagaaggcgcgcaacgtggtcatgttcctgggcgacggcatgtccgtgcccacgctcgccgccgcgcggacgctgctcggccagcgccgcgggcacaccggcgaggaggataaactgcattttgaaacattccccaccgttggattgactaagacgtattgcgtgaacgctcagatcccagactccgcgtgcactgctactgcgtacttatgtggtgtcaaaacaacttacggagctattggagtgaatgcggaggtgccacggaaaggctgcgaggcgtccaccgacaccagccgacacgtggagtccatcgcggagtgggcgctggccgacggccgcgacgctggtatcgtgacgacgacgcgcatcacccacgcgtcgccggccggcgtgtacgccaaggtggcggaccgcaactgggagcacaaccaggcggtggagaacgatggcttcgacacggacaagtgcccggatatcgcactgcagctcgtgcataagcaccccgggaataaactcaaggttattttaggcggaggaagactaaactttttgccaaatgatgtgaaagacgaagaaggggtatatggaaaccgaacagacagccgcaacctcatcgaagaatgggcacaagacaaggaagatcgtaaagttactcataaatatgtttggaatcgtgagcagctgatgagtcttaaagatgatcttcctgagtaccttttaggacttttcgaaagtaatcatcttcagtacaacatgcaggcagatcctaatactgagcccacgctgactgagctaactgagatagcaatcaagtcgctaagtaraaacgagaaaggttttttcctgttcgtggaaggcggtcgtatcgaccacgcgcaccatcgcaactgggtagagctagcgctggacgagaccctggagatggacgaggccgtcgcgcgcgccgccgagctgctccccgaggacgactcgcccattgtggtcacagcnnaccactcccacgtcatggcttacaatggatactcggcccgtggacatgacatcctcggcccttccagagacttggacctggacggagtgccttacatgacgctgtcgtacaccaacgggcccggcttccgttcgcatatgaacggtatacgccccgatgtcaccgctgaagacggtttcggagaagacgaatggttggctcatgtagatgttccgttgatagacgagacgcacggcggggacgacgtggcggtgttcgcgcgcgggccgcaccactccatgttcacggggctgtacgagcagagccagctgccgcacctcatggcgtacgccgcctgcatcggccccggcagacacgcctgcagcggcgccgcgcatgcgctggcccagcctgtgctgctgctctctctccttgtactgctcacttcactattccaacaatga

SEQ ID NO: 22 - protein, 541 aa, Spodoptera frugiperda
  misc_feature (339)..(339): Xaa can be any naturally occurring amino acid
  misc_feature (395)..(395): Xaa can be any naturally occurring amino acid
MRSLLTYLVAAVVVAACVRGDRYHPADPGSRADTVANRAETSANYWAQEAQAAINARLAHKESVKKARNVVMFLGDGMSVPTLAAARTLLGQRRGHTGEEDKLHFETFPTVGLTKTYCVNAQIPDSACTATAYLCGVKTTYGAIGVNAEVPRKGCEASTDTSRHVESIAEWALADGRDAGIVTTTRITHASPAGVYAKVADRNWEHNQAVENDGFDTDKCPDIALQLVHKHPGNKLKVILGGGRLNFLPNDVKDEEGVYGNRTDSRNLIEEWAQDKEDRKVTHKYVWNREQLMSLKDDLPEYLLGLFESNHLQYNMQADPNTEPTLTELTEIAIKSLSXNEKGFFLFVEGGRIDHAHHRNWVELALDETLEMDEAVARAAELLPEDDSPIVVTAXHSHVMAYNGYSARGHDILGPSRDLDLDGVPYMTLSYTNGPGFRSHMNGIRPDVTAEDGFGEDEWLAHVDVPLIDETHGGDDVAVFARGPHHSMFTGLYEQSQLPHLMAYAACIGPGRHACSGAAHALAQPVLLLSLLVLLTSLFQQ

SEQ ID NO: 23 - DNA, 4020 bp, Heliothis virescens
atgggcgtagaaaataagaataatgtacaaaatgcggaaggcgcggccctcaagacttacaagaaaccgaacattttatctcgtatatttctttggtggatgtgtccggtgctcataactggtaacagaagaaacgtagaagaatcagatctaataccgcccagtaatttatataattcagaaagacaaggagaatatctagaaagatactggttacaagaaatagaaaatgcaacaaatgaaaatcgggagccatcgctatggaaggcgttgcaaagggcctactgggtatcctatatgccaggagccatttatgtcttaattcaatcagcagccaggacgtatcagccgttgttatttgctcagctactgacgtactggtcggtcgatagtgaaatgactcagcaagacgctgggctgtatgcgctagccatgctgggactgaacttcgtctccatgatgtgtcagcaccacaacaacttgtttgtgatgcggttcagtttgaaagtcaaggttgcttgttcttcactcttgtataggaagttgctccgcatgactcaagtgtcggtaagtgaagtcgcaggtggaaagttggtaaacttgctgtccaacgacatcacgaggttcgactacgcattcatgttcctgcactacttgtggatagtgcctatccaagtggccgtagtcttgtacttcttgtgggatgctgctgggtttgcgccttttgttggtctctttggagttgtcctattgattttaccacttcaagccggtttgacgagactcacatctattgtaaggcgtgagacagccaagaggacggataggcgaattaaacttatgagtgaaattatcaatggtattcaggtcattaaaatgtacgcttgggagaaacccttccagctagtggtgaaggcggctcgtgcctttgaaatgagtgctctcaggaagtccatcttcattaggagtaccttcctcgggttcatgttgttcactgagcggagtatcatgtttgtcacagtattgacactcgcactcacaggcactatgattactgctaccacgatatatcccattcagcagtacttcagtattattcaatttaatgtaacgctagtcattcctatggcaatcgcaagtttatccgagatgatggtgtctatagaacgtatccaaggattcctcagtttggatgagcggtccgacatgcaagtgactcctaaaatgaatggctccaataacagcactttgttcaaaagcaagaaggcaccacttgaaataagcatcgtgccaaagaaatactcgcctagcgaagttacggttgcaagagaagtgcaggatgatcccagccaggtggactatcctataagactcaacaaaataaacgcatcgtggaccggcaatgatactccttcagagatgacacttaaaaatatatccttacgtatacgtaaaggcaaattgtgtgctatcattggtcctgtgggttccggaaagacgtctctgcttcagctactcttaaaagaattaccgatgactagtggcacactggacgtgtcaggaagattgtcttacgcttgtcaggagtcgtggctgttccccggcacagtgcgagaaaacattttgtttggcctagattatgaagccacaaaatacaaagaggtttgcaaggtgtgttcgttactgccagacttcaaacagttcccgtatggtgacttgtctttagtgggggagcgaggagtatccctgtctggaggtcaaagagccagaatcaatttagccagagcaatttatcgtgaggccgatatttacttgctggatgatcccctatccgcagtggatgcaaatgtaggcagacaactgtttgatggctgcatcaaaggctacctttctggacgaacttgcgtcttggtcacccatcagatccattacctcaaagctgctgattttattgtagtccttaatgagggttccatcgagaacatgggcacgtatgacgagctggtcaagacaggaaccgagttctcgatgctgctatccaaccaagaaagtgaagcaactgaaaacgaaatgaaagagcgaccatcattgctgcgaggaatatcaaaaatctcaattaagagcgacgaccacgatgcggatcagaaggcgcaagtacaggaggcagaggagagagcaacaggcagcttgaagtgggaggtggtgctgaagtacctgagctccgtcgaatcgtggtgtctggtgttcatggctttcctcgcgctgctgatcacgcaaggcgctgccactacgtctgactattggctgagtttctggactaatcaagtggattcttatgaacaatcattgcctgatggcgccgaaccagatactgacatgaacgcacaaattggtttacttacaactgcccagtacctatacgtgtacggcggagttatattggctgtaataatcatgacactcgtcaggatcacaggtttcgtagcgatgacaatgcgagcctctcaaaatctccacaacactatttacgaaaaattgattgtgacagtaatgagattctttgataccaatccttctggtcgtgtcctgaacaggttctcaaaagacatgggtgccatggatgagcttctacctcgcagtcttttagaaacagttcagatgtatctgtcgctgaccagcatcttggtgctaaacgccacagcattgccctggacgctcatacctacctccgtattgatagtcatcttcgtgttcatgttgagatggtacctgaatacagctcaggctgtcaaacgattggaaggaacaaccaagagtcctgtatttggaatgattaattccactatttctggactttcgactatcaggagttcgggttcccagtttagacagatgagattatttgacgaagcgcagaatctccacacaagtgctttccacacattctttggcggttctacggcatttggattgtatctcgatactttgtgtttgatctacctcggagttgtcatgtcaattttcattttgggcgactttggtgatttgatcgcagtaggaagcgtcggtctggccgtcagtcagtccatggtgctcaccatgatgttgcagatgacagctaggtttacagctgacttcttgggacaaatgacggcagtagagagggtgctggagtacacccagctacccatggagactaatatggagcaaggaccaactaacccgccaaaagaatggcctaatgctggcagagtgacgttctcaaatgtgtacctgaattattctgtggaagacccaccagtgctaaaggacttgaactttgaaattcaaagcggttggaaggttggagttgtaggccgaacgggagccggcaaatcatcgctcattgcggctctgttccggcttaccgacataactggcagcatcaaaattgacggcgtggacacagaaggattagccaaaaagcttttgagatcgaaaatatcaataattccacaagagccggtcctcttctccgctactctgcgttataatttggatccgttcgacgattacagtgacgaagatatttggagggctctggaacaggtggaactaaaagaaggaataccggcacttgattataaagtggctgaaggtggtactaacttctccatgggacagcgtcagttggtatgcttggctcgtgctattttacgctctaacaaaatactcatcatggacgaagccacagctaatgtcgatcctcagacggacgctttgattcaaaagacgatccgtcgtcaattcgcgtcgtgcacggtgctgaccatcgcgcatcgactgaacaccatcatggactccgaccgagtgctggtcatggaccagggcgaggtcgccgagttcgatcacccacacatcttgctcagcaaccccaacagcaagttcttctctatggtcagagagaccggagaaagcatgacgaagaccttaatggaggtcgcgaagactaaatacgatagtgataataaggaggcttag

SEQ ID NO: 24 - protein, 1339 aa, Heliothis virescens
MGVENKNNVQNAEGAALKTYKKPNILSRIFLWWMCPVLITGNRRNVEESDLIPPSNLYNSERQGEYLERYWLQEIENATNENREPSLWKALQRAYWVSYMPGAIYVLIQSAARTYQPLLFAQLLTYWSVDSEMTQQDAGLYALAMLGLNFVSMMCQHHNNLFVMRFSLKVKVACSSLLYRKLLRMTQVSVSEVAGGKLVNLLSNDITRFDYAFMFLHYLWIVPIQVAVVLYFLWDAAGFAPFVGLFGVVLLILPLQAGLTRLTSIVRRETAKRTDRRIKLMSEIINGIQVIKMYAWEKPFQLVVKAARAFEMSALRKSIFIRSTFLGFMLFTERSIMFVTVLTLALTGTMITATTIYPIQQYFSIIQFNVTLVIPMAIASLSEMMVSIERIQGFLSLDERSDMQVTPKMNGSNNSTLFKSKKAPLEISIVPKKYSPSEVTVAREVQDDPSQVDYPIRLNKINASWTGNDTPSEMTLKNISLRIRKGKLCAIIGPVGSGKTSLLQLLLKELPMTSGTLDVSGRLSYACQESWLFPGTVRENILFGLDYEATKYKEVCKVCSLLPDFKQFPYGDLSLVGERGVSLSGGQRARINLARAIYREADIYLLDDPLSAVDANVGRQLFDGCIKGYLSGRTCVLVTHQIHYLKAADFIVVLNEGSIENMGTYDELVKTGTEFSMLLSNQESEATENEMKERPSLLRGISKISIKSDDHDADQKAQVQEAEERATGSLKWEVVLKYLSSVESWCLVFMAFLALLITQGAATTSDYWLSFWTNQVDSYEQSLPDGAEPDTDMNAQIGLLTTAQYLYVYGGVILAVIIMTLVRITGFVAMTMRASQNLHNTIYEKLIVTVMRFFDTNPSGRVLNRFSKDMGAMDELLPRSLLETVQMYLSLTSILVLNATALPWTLIPTSVLIVIFVFMLRWYLNTAQAVKRLEGTTKSPVFGMINSTISGLSTIRSSGSQFRQMRLFDEAQNLHTSAFHTFFGGSTAFGLYLDTLCLIYLGVVMSIFILGDFGDLIAVGSVGLAVSQSMVLTMMLQMTARFTADFLGQMTAVERVLEYTQLPMETNMEQGPTNPPKEWPNAGRVTFSNVYLNYSVEDPPVLKDLNFEIQSGWKVGVVGRTGAGKSSLIAALFRLTDITGSIKIDGVDTEGLAKKLLRSKISIIPQEPVLFSATLRYNLDPFDDYSDEDIWRALEQVELKEGIPALDYKVAEGGTNFSMGQRQLVCLARAILRSNKILIMDEATANVDPQTDALIQKTIRRQFASCTVLTIAHRLNTIMDSDRVLVMDQGEVAEFDHPHILLSNPNSKFFSMVRETGESMTKTLMEVAKTKYDSDNKEA

SEQ ID NO: 25 - DNA, 4017 bp, Helicoverpa armigera
atgggcgtagaaaataagaataatgtacaaaatgcggaaggtccggcccgcaagacttacaaaagaccgaacattttatcccgtatatttctctggtggatgtgtcctgtgctcataactggtaacaaaagaaatgtagaagaatcagatcttataccgcccagtaatttatataattcagaaagacaaggagaatatcttgaaagatactggttggcagagatagaaaatgcaacaattgaaaatcgagagccatcactttggaaggcattacgaaaggcctactgggtctcctatatgccaggagctatttttatcatcattcaatctgcagccaggacgtatcagccgctgttgttttctcagcttttgtcgtactggtcagtggacagtgaaatgactcagcaagacgctggcctgtatgctctcgccatgctgggactgaacttcgtctctatgatgtgtcagcaccacaacacattgtttgtgatgcggttcagtttaaaagtcaaggttgcctgttcttcgctcttgtataggaagttgctccgcatgacccaagtgtcagtaggtgaggtggctggtggaaagttggtgaacttgctgtccaacgatatcacgaggttcgactacgccttcatgttccttcactacttgtggatcgtgcctatccaagtggctgttgtcttatacttcttgtgggaagctgctggctttgcgcccttcgtcggtctgtttggagtcgttatactgattttaccactgcaagctggcttgacgaaactcacaactgttgtaagacgtgagacggctaagagaacggacaggcgaattaaactaatgagtgaaattattggtggtattcaggtcattaaaatgtacgcttgggagaaacccttccagctagttgtgaaagcagcccgtgccttcgaaatgggtgccctcaggaagtccatcttcatcaggagtactttcctagggttcatgttgttcactgaaagaagcatcatgtttgtcacagtgttgacactcgctctcacaggcactatgattactgctaccacgatatatcctattcaacagtacttcagtattattcagtttaacgtaacactgatcattcctatggcaatcgcaagttattccgagatgatggtgtccatagaacgtatccagggattccttagtttggacgagcggtcagacatgcaagtgactccaaaaatgaatggatctaacaataacactttgtttaaatccaagaagtcaccacttgaagtaggcatcgtgcctaaaaaatactcaccaagcgaagttatggctgcaaaggagatgcaggatgatcctacccagatggactatcctatcagactcaacaaagtaagcgcatcctggaccggcagcaatagttcttcagaaatgacacttaagaatatatcgttgcgtatacgtaaaggaaaattgtgtgctatcattggtcctgtggggtccggaaagacgtctctactgcaactgctgttaaaagaattaccattgaacagtggtacacttgacgtgagtgggaagatgtcctacgcttgtcaagagtcctggctgttcccgggcacagtgcgagaaaacattttgtttggcctaacttacgaacccacaaaatacaaggaggtttgcaaggtgtgttcgttactgccggatttcaagcagttcccgtatggtgacctgtccttggtgggagagagaggagtatccttgtcaggtggtcaaagagccaggatcaatttggccagagcaatttatcgtgaggccgacatttacttgctggatgatcctctatctgcagtggacgctaatgtaggcagacaactgtttgacggctgcatcaaaggctacctcactggaagaacgtgcgtcttggtcacccatcagatccattacctcaaagctgctgattttattgtagtccttaatgagggctccgtcgagaatatgggcacgtatgatgagctggtgaagacaggaactgagttctcaatgctgctatccaaccaagaaaatgacgcaactgaaaacgaaaagaaagatcgaccagcaatgatgcgaggaatatcaaaaatctcagttaagagcgacaccgaaatggaacagaaggctcaaatacaggaggcagaggagagagcgacaggtagcttgaagttcgaggtggtgctcaagtatctgagctcagtccagtcctggtgtctggtgttcacggcgttcctggtgctgctgatcacgcaaggcgctgccaccacggccgactattggttgagtttctggactaatcaagtggattcttatgaacaatcgttgcctgatggcgttgatccagatactgacatgaatgcacaaattggtttacttacaactgcccagtacctatacgttttcggtggagttatattggctttgatagtcatgaccctcgtcagaatcacagctttcgtagcgatgacaatgcgagcttctcaaaatcttcacaacactatttacgaaaaattgattgtgactgtaatgagatttttcgatactaatccatctggtcgtgtcctgaacaggttctcaaaagacatgggtgctatggatgagcttctacctcgcagtcttttagaaacaattcagatgtatctgtctctgaccagcgtgttggtgctaaacgccacagcattgccctggacgctcattcctacctccgtgttgattgtcatcttcgtgctcatgttgagatggtacctgaatactgctcaggctgtgaaacgtttggaaggcacaaccaagagtcctgtatttggaatgattaactccactatttctggactctccactatcagaagttctggttcccaggatagacagatgaaattgtttgacgaagcgcagaatctccacacaagtgctttccacacattcttcggcggttctacggcatttgcactgtatctcgatactttgtgtttgttctacctcggagttgtcatgtcaatcttcattttgggcgactttggtgatttgatcccggtgggaagcgtcggtctggccgtcagtcagtccatggtgctgaccatgatgttgcagatggcagccaggtttacagctgacttcttgggacaaatgacggcagtggagagagtgctggagtacaccaaactacccacggaaaccaatatggaacaaggaccaactaacccaccaaaagaatggcccagtgctggtagagtgacgttctcaaatgtgtacctgaattattccatggaagacccaccggtgctgaaggacttaagctttgaaattcaaagcggttggaaggttggagttgtaggcagaactggagccggcaagtcatcactcatcgcggctttgttccggcttagcgacataagcggcagcatcaaaattgacggcgtggacaccgaaggattagccaaaaagactttgagatcgaaaatatcaattattccacaagaaccggtgctgttctcggctactctgcgatacaatttggatccattcgacgattacagcgacgacgatatttggagggcgttggaacaggtggaattaaaagaaggaataccggctttagactttaaggtcgctgaaggtggtactaacttctctatgggacaacgtcaactggtgtgcttggcgcgtgccattcttcggtctaataaaatactcatcatggacgaagctaccgctaatgtcgatcctcagacggacgctttgatccaaaagacgatccgtcgccagttcgcgtcgtgcacggtgctcaccatcgcgcatcgactgaacaccatcatggactccgaccgagtgctggtcatggaccagggcgaggtggccgagttcgaccacccccacatcttgctcagcaaccccaatagcaagttcttctctatggtccgggagacaggagaaagcatgacaaggaccttaatggaggtcgctaaggccaaatatgatagtgataataaggaggcttaa

SEQ ID NO: 26 - protein, 1338 aa, Helicoverpa armigera
MGVENKNNVQNAEGPARKTYKRPNILSRIFLWWMCPVLITGNKRNVEESDLIPPSNLYNSERQGEYLERYWLAEIENATIENREPSLWKALRKAYWVSYMPGAIFIIIQSAARTYQPLLFSQLLSYWSVDSEMTQQDAGLYALAMLGLNFVSMMCQHHNTLFVMRFSLKVKVACSSLLYRKLLRMTQVSVGEVAGGKLVNLLSNDITRFDYAFMFLHYLWIVPIQVAVVLYFLWEAAGFAPFVGLFGVVILILPLQAGLTKLTTVVRRETAKRTDRRIKLMSEIIGGIQVIKMYAWEKPFQLVVKAARAFEMGALRKSIFIRSTFLGFMLFTERSIMFVTVLTLALTGTMITATTIYPIQQYFSIIQFNVTLIIPMAIASYSEMMVSIERIQGFLSLDERSDMQVTPKMNGSNNNTLFKSKKSPLEVGIVPKKYSPSEVMAAKEMQDDPTQMDYPIRLNKVSASWTGSNSSSEMTLKNISLRIRKGKLCAIIGPVGSGKTSLLQLLLKELPLNSGTLDVSGKMSYACQESWLFPGTVRENILFGLTYEPTKYKEVCKVCSLLPDFKQFPYGDLSLVGERGVSLSGGQRARINLARAIYREADIYLLDDPLSAVDANVGRQLFDGCIKGYLTGRTCVLVTHQIHYLKAADFIVVLNEGSVENMGTYDELVKTGTEFSMLLSNQENDATENEKKDRPAMMRGISKISVKSDTEMEQKAQIQEAEERATGSLKFEVVLKYLSSVQSWCLVFTAFLVLLITQGAATTADYWLSFWTNQVDSYEQSLPDGVDPDTDMNAQIGLLTTAQYLYVFGGVILALIVMTLVRITAFVAMTMRASQNLHNTIYEKLIVTVMRFFDTNPSGRVLNRFSKDMGAMDELLPRSLLETIQMYLSLTSVLVLNATALPWTLIPTSVLIVIFVLMLRWYLNTAQAVKRLEGTTKSPVFGMINSTISGLSTIRSSGSQDRQMKLFDEAQNLHTSAFHTFFGGSTAFALYLDTLCLFYLGVVMSIFILGDFGDLIPVGSVGLAVSQSMVLTMMLQMAARFTADFLGQMTAVERVLEYTKLPTETNMEQGPTNPPKEWPSAGRVTFSNVYLNYSMEDPPVLKDLSFEIQSGWKVGVVGRTGAGKSSLIAALFRLSDISGSIKIDGVDTEGLAKKTLRSKISIIPQEPVLFSATLRYNLDPFDDYSDDDIWRALEQVELKEGIPALDFKVAEGGTNFSMGQRQLVCLARAILRSNKILIMDEATANVDPQTDALIQKTIRRQFASCTVLTIAHRLNTIMDSDRVLVMDQGEVAEFDHPHILLSNPNSKFFSMVRETGESMTRTLMEVAKAKYDSDNKEA

SEQ ID NO: 27 - DNA, 795 bp, Artificial Sequence
  SYNTHESIZED
  misc_feature (480)..(480): n is a, c, g, or t
aggtccaactgcaggagtctgggggaggctcggtgcagtctggagggtctctgagactctcctgtgcagcctctggatacaccatcactaatagttaccgcatggcctggttccgccaggctccagggaaggagcgcgagggggtcgcagctatcaatagtggtggttctacaacatacgcagactccgtgaagggccgattcaccatctcccaagacaacgcccagaacacgctgtatttgcagatgaacagcctgaaagccgaggacacggccatgtattactgtgcggaggaggaggaagaggaggaggaggaggcagggcgcgtacagtggtggcctgtactacgcgcgctgaacgaggatgattatctctactggggccaggggacccaggtcaccgtctcctcavcaggtgcagctgcaggagtcggggggagactcggtgcaggctggagggtctctgagactcgcctgtgcngcctcggcctctggctactccgtgtgtgtggggtgggtgggctggttccgcccggctcccgggcgggagcgcgggggggtcgccgttgtttctgttcctggtggtggttccttctttggcggcgacgtggggggccgattttccctctccccggaccacgcccagaacacggtgtatccgcaaatgaacagtctgaaacctgaggacactgccatgtactattgcgcagcgcgcaatgcgggggggcgttttcggccttcggccaatggtgggtataattattggggccaggggacccaggtcaccgtctcctca

SEQ ID NO: 28 - protein, 125 aa, Llama glama
QVQLQESGGRSVQSGGSLRLSCAASGIDVNRNAMGWFRQAPGTEREFVAGVRWSDAYTDYADSVKGRFTISRDNNKNTVYLQMGSLEAGDTALYYCAAGLLDVQYVRQAAGYSYWGQGTQVTVSS

SEQ ID NO: 29 - protein, 265 aa, Artificial Sequence
  SYNTHESIZED
QVQLQESGGGSVQSGGSLRLSCAASGYTITNSYRMAWFRQAPGKEREGVAAINSGGSTTYADSVKGRFTISQDNAQNTLYLQMNSLKAEDTAMYYCAAGRVQWWPVLRALNEDDYLYWGQGTQVTVSSGGGGSGGGGQVQLQESGGDSVQAGGSLRLACAASASGYSVCVGWVGWFRPAPGRERGGVAVVSVPGGGSFFGGDVGGRFSLSPDHAQNTVYPQMNSLKPEDTAMYYCAARNAGGRFRPSANGGYNYWGQGTQVTVSS

SEQ ID NO: 30 - protein, 57 aa, Spodoptera frugiperda
LPTLPWSVCQEEWGDCLPSDPDLDPVGTITNETKSSAELYFLKTVLQQKDGIEDGLG

SEQ ID NO: 31 - protein, 635 aa, Spodoptera frugiperda
MKMSLRLTLKTVKMSGETNNGFDASPEDGKAAALNDKSLVNGANEKSPEKKDEPERAVWGNQIEFLMSCIATSVGLGNVWRFPFVAYQNGGGAFLIPYIIVLLLIGKPMYYLECVLGQFSSKNSVTVWSLSPAMKGAGYATALGCGYILSYYVSIVALCLYYLAMSFLPTLPWSVCQEEWGDCLPSDPDLDPVGTITNETKSSAELYFLKTVLQQKDGIEDGLGLPIWYLVVCLFGSWFIIFVIVSRGVKSSGKAAYFLALFPYVVMLILLITTSILPGAGTGILFFLTPQWDKLIELDVWYAAVTQVFFSLSVCTGAIIMFSSYNGFRQNVYRDAMIVTTLDTFTSLLSGFTIFGILGNLAYELDKDVDDVTGSAGTGLAFISYPDAISKTFQPQLFAVLFFLMMTVLGIGSAVALLSTINTVMMDAFPRIKTIYMSAFCCTIGFAIGLIYVTPGGQYILELVDYFGGTFLILFCAIAEIIGVFWIYGLENLCLDIEYMLGVKTSFYWRCCWGVIMPAMMITVFIYALATSETLKFGEDYYYPTAGYVAGYMMLFVGVAFVPISIGLTMYKNKTGDCAETAKRSFRPKESWGPREEFERLNWIEFRREAEAERAQKRTSWLQHIRYSLFGGYRR

SEQ ID NO: 32 - protein, 352 aa, Spodoptera frugiperda
MGAMFRSEEMALCQLFIQPEAAYTSVSELGEAGSVQFRDLNPDVNAFQRKFVNEVRRCDEMERKLRYIEAEVHKDGVHIPAVKEAPRAPNPREIIDLEAHLEKTENEILELSHNAVNLKQNYLELTELRHVLEKTEAFFIAQEEIGMDSMTKSLISDETGQQAATRGRLGFVAGVVNRERVPAFERMLWRISRGNVFLRRAELDKPLEDPATGNEIYKTVFVAFFQGEQLKSRIKKVCSGFHASLYPCPPSNTERQDMVKGVRTRLEDLNMVLNQTSDHRQRKALADGSNACGSSIPSFLNCIETDEEPPTFNRTNRFTRGFQNLIDAYGVASYRECNPALYTTITFPFLFA

SEQ ID NO: 33 - protein, 788 aa, Spodoptera frugiperda
MGAMFRSEEMALCQLFIQPEAAYTSVSELGEAGSVQFRDLNPDVNAFQRKFVNEVRRCDEMERKLRYIEAEVHKDGVHIPAVKEAPRAPNPREIIDLEAHLEKTENEILELSHNAVNLKQNYLELTELRHVLEKTEAFFIAQEEIGMDSMTKSLISDETGQQAATRGRLGFVAGVVNRERVPAFERMLWRISRGNVFLRRAELDKPLEDPATGNEIYKTVFVAFFQGEQLKSRIKKVCSGFHASLYPCPPSNTERQDMVKGVRTRLEDLNMVLNQTSDHRQRKALADGSNACGSSIPSFLNCIETDEEPPTFNRTNRFTRGFQNLIDAYGVASYRECNPALYTTITFPFLFAVMFGDLGHGCIMALFGAWMVCNEVKLAAKKSNNEIWNIFFAGRYIILLMGCFSMYTGLVYNDIFSKSMNIFGSSWSVPYDNDTMEHNAALTMDPKTSYNNNPYFIGIDPVWQSADNKIIFLNSYKMKLSIIFGVLHMIFGVCMSVVNYNFFRRRYSIVLEFLPQVIFLCLLFLYMVFMMFYKWVAYSAFSEEQAYTPGCAPSVLILFINMMLFSSTAPPEGCNEYMFEAQASIQRVFVLVALCCIPVMLLGKPLYLLAAGKKKKEAKPEHSNGSVNPGIEMQEQTDIGDGVPKPEAKPAASGHDHEDEPFSEIMIHQAIHTIEYVLSTISHTASYLRLWALSLAHAELSEVLWNMVLTFGLKDHDYIGGIKLYVAFCFWALFTLAILVMMEGLSAFLHTLRLHWVEFMSKFYSGLGYIFQPFCFKTILEQEDNKDD

SEQ ID NO: 34 - protein, 1174 aa, Spodoptera frugiperda
MENNIQNQCVPYNCLNNPEVEILNEERSTGRLPLDISLSLTRFLLSEFVPGVGVAFGLFDLIWGFITPSDWSLFLLQIEQLIEQRIETLERNRAITTLRGLADSYEIYIEALREWEANPNNAQLREDVRIRFANTDDALITAINNFTLTSFEIPLLSVYVQAANLHLSLLRDAVSFGQGWGLDIATVNNHYNRLINLIHRYTKHCLDTYNQGLENLRGTNTRQWARFNQFRRDLTLTVLDIVALFPNYDVRTYPIQTSSQLTREIYTSSVIEDSPVSANIPNGFNRAEFGVRPPHLMDFMNSLFVTAETVRSQTVWGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGTFDECYPTYLYQKIDESKLKPYTRYQLRGYIEDSQDLEIYLIRYNAKHETVNVLGTGSLWPLSVQSPIRKCGEPNRCAPHLEWNPDLDCSCRDGEKCAHHSHHFSLDIDVGCTDLNEDLDVWVIFKIKTQDGHARLGNLEFLEEKPLVGEALARVKRAEKKWRDKREKLELETNIVYKEAKESVDALFVNSQYDQLQADTNIAMIHAADKRVHRIREAYLPELSVIPGVNVDIFEELKGRIFTAFFLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTANQEEYGGAYTSRNRGYDETYGSNSSVPADYASVYEEKSYTDGRRDNPCESNRGYGDYTPLPAGYVTKELEYFPETDKVWIEIGETEGTFIVDSVELLLMEE

SEQ ID NO: 35 - protein, 427 aa, Spodoptera frugiperda
GEFLDRLSATDEDGLHAGRVTFSIAGNDEAAEYFNVLNDGDNSAMLTLKQALPAGVQQFELVIRATDGGTEPGPRSTDCSVTVVFVMTQGDPVFDDNAASVRFVEKEAGMSEKFQLPQADDPKNYRCMDDCHTIYYSIVDGNDGDHFAVEPETNVIYLLKPLDRSQQEQYRVVVAASNTPGGTSTLSSSLLTVTIGVREANPRPIFESEFYTAGVLHTDSIHKELVYLAAKHSEGLPIVYSIDQETMKIDESLQTVVEDAFDINSATGVISLNFQPTSVMHGSFDFEVVASDTRGASDRAKVSIYMISTRVRVAFLFYNTEAEVNERRNFIAQTFANAFGMTCNIDSVLPATDANGVIREGYTELQAHFIRDDQPVPADYIEGLFTELNTLRDIREVLSTQQLTLLDFAAGGSAVLPGGEYALAVYI

SEQ ID NO: 36 - protein, 9 aa, Artificial Sequence
  SYNTHESIZED
GGGGSGGGG

SEQ ID NO: 37 - protein, 796 aa, Spodoptera frugiperda
SPESASTTPTPAPIVSPTTDSDGSSGTTEGPTSPLTPSTTPSTTSTTTTTTTTTTTTTTTTTETPPDESPGQLDLEEIIDGVFSPPSFNATWATGSELMFRNDNGDLVLYDVDSDTPMPIVSNTSKILQEASRVMQLSPEGKDVMLAHSVAPVYRYSFIARYTAVNIENEEEVPITPPSVPREQALLQNLVWGPSGTSLAFVYYNNIYYQPSLKEAPRQITTDGVLNVIYNGIPDWVYEEEVFGSNNAIWFSKDGTKMAYVTFDDSHVEVMRVPHYGIPGETQYTRHRQIRYPKPNTTNPTVKVTLWNLITDTPSVVEAPNDLNQPILKTVKFINNDDIAVVWTNREQTSLRVQKCRASGNTAECTTIYNYVENGGWIDNIPFFFNDAGNSFITILPFAVDGVRFKQIVQVTEGTATAPATVKNRVNNPHTVLEILAWGTDDVIWYKATSVSDSREQHIFSVNSQDVISCFTCNIRRTDGGLCLYNEGTISTAGDRITINCAGPDVPQIFIYKTNGELVRVWDEGADLSNLMHNRTLPVTLRAQITSPLGQASTDIHIQAPADYAHRTNVPLLVYVYGGPDTALVTRQWSLDWGSSLVSRWGIAVAHIDGRGSGLRGVENMFALNRKLGTVEIEDQIAGAKYRYIQDNFPWIDANRTCIWGWSYGGYAASKALAEGGDVFRCAAAIAPVVDWRFYVDTIYTERYMGLPTAEDNAEGYEVSSLLTKAEALREKSYFLVHGTADDNVHYQHAMLLSRLLQRRDVFFTQMSYTDEDHGLVGVRPHLYHALERFLQEYML

SEQ ID NO: 38 - protein, 847 aa, Spodoptera frugiperda
MAQDNSNSTMEMGTSDQVLVATKRKKTIGYIIGTIAMLAVGGVVIALIVVLSPESASTTPTPAPIVSPTTDSDGSSGTTEGPTSPLTPSTTPSTTSTTTTTTTTTTTTTTTTTETPPDESPGQLDLEEIIDGVFSPPSFNATWATGSELMFRNDNGDLVLYDVDSDTPMPIVSNTSKILQEASRVMQLSPEGKDVMLAHSVAPVYRYSFIARYTAVNIENEEEVPITPPSVPREQALLQNLVWGPSGTSLAFVYYNNIYYQPSLKEAPRQITTDGVLNVIYNGIPDWVYEEEVFGSNNAIWFSKDGTKMAYVTFDDSHVEVMRVPHYGIPGETQYTRHRQIRYPKPNTTNPTVKVTLWNLITDTPSVVEAPNDLNQPILKTVKFINNDDIAVVWTNREQTSLRVQKCRASGNTAECTTIYNYVENGGWIDNIPFFFNDAGNSFITILPFAVDGVRFKQIVQVTEGTATAPATVKNRVNNPHTVLEILAWGTDDVIWYKATSVSDSREQHIFSVNSQDVISCFTCNIRRTDGGLCLYNEGTISTAGDRITINCAGPDVPQIFIYKTNGELVRVWDEGADLSNLMHNRTLPVTLRAQITSPLGQASTDIHIQAPADYAHRTNVPLLVYVYGGPDTALVTRQWSLDWGSSLVSRWGIAVAHIDGRGSGLRGVENMFALNRKLGTVEIEDQIAGAKYRYIQDNFPWIDANRTCIWGWSYGGYAASKALAEGGDVFRCAAAIAPVVDWRFYVDTIYTERYMGLPTAEDNAEGYEVSSLLTKAEALREKSYFLVHGTADDNVHYQHAMLLSRLLQRRDVFFTQMSYTDEDHGLVGVRPHLYHALERFLQEYML

SEQ ID NO: 39 - protein, 209 aa, Spodoptera frugiperda
LPTYGTPVSEGLAQLRVYNGYNCNFTLNTANLNTLEENATRDFEIGPLSAYENLHIFADDFVDLPYYLQGEPATECAGIAYSGYFNLKEKTANSFFIKKDGLYNFTDNNDKAIDGVNVRFLSNINSVVDISIENDQKNKTLLSIQSNDTSQKSIAKGVSNVTVGGFVVLSDFNFKSGAVYTINIYEDRAGVYYANTVMITPSNSIHILW

SEQ ID NO: 40 - protein, 584 aa, Spodoptera frugiperda
MEFTIVALLLIAFGTGGIKPCVSAFGGDQFKLPEQERYLGYFFSLFYFAINAGSLISTFLTPILRADVHCFGDNDCYSLAFGVPGILMVVSIVFFVAGKRLYVIKKPAGNVLGKVSTCIGHAVVKSCKSKEKREHWLDHADDKYDSNLIEDVKALLRVLVLFIPLPVFWALFDQQGSRWTFQADRMEQDIGSWTLKADQMQVLNPLLILIFIPIFEVAIYPFLTWCKLVRKPLHKMIWGGILAACAFIISGIVELNLLPTYGTPVSEGLAQLRVYNGYNCNFTLNTANLNTLEENATRDFEIGPLSAYENLHIFADDFVDLPYYLQGEPATECAGIAYSGYFNLKEKTANSFFIKKDGLYNFTDNNDKAIDGVNVRFLSNINSVVDISIENDQKNKTLLSIQSNDTSQKSIAKGVSNVTVGGFVVLSDFNFKSGAVYTINIYEDRAGVYYANTVMITPSNSIHILWLIPQYVVMTMGEVMFSVTGLEFSFTQAPASMKSVLQSVWLLTVAFGNLIVVLIVEGNFLDAQWKEFFLFAGLMLIDMLIFTTMAFRYKYKELSSSDENLAIEEIKMPEKTSQDKHEKN

SEQ ID NO: 41 - DNA, 23 bp, Llama glama
gtcctggctgctcttctacaagg

SEQ ID NO: 42 - DNA, 23 bp, Llama glama
ggtacgtgctgttgaactgttcc

SEQ ID NO: 43 - protein, 1260 aa, Spodoptera frugiperda
MKDSKGNAWQLKPGEKENENKLPDPLNEDVQKGQFSDTSSEPSEAKAEEVPSIPFITLFRFASTRDKLFIICALICSAVAAISTPLNTLLLAYLLEAMVNYSIFGDADAFMKSLLNFAIYNAAVGAALVVLSYAATTLMNIAAYNQVYVIRQEYLKAALNQDFGYFDVHKNAEIANKMNSDIMKLEEGIGEKLATFFFYQASFLSSVIMALVKGWKLALLCLISFPVTMTLVGVAGLIAASLSKKEAIATGKAGAIAEEVISAIRTVYAFSGQEKELDRYEGHLNDARKINVKKSLFNGLAMGCLFFCIFCAYALSFWFGYRLMVTDGYDVSTMIAVFFGVMTGSANFGISSTLMEVFGSARGAGAHIFNMIDNVPTINPLQNRGTVPSDIEGNIELKNVEFHYPSRPDVPVLKGVSIKVKRGQSVALVGHSGCGKSTIIQLISRFYDVVEGSVAIDGNDVRDLSVRWLREQIGLVGQEPVLFNTTVRENIRYGRENATNEEIEACARQANAHQFIMKLPKGYDTLVGERGASLSGGQKQRIAIARALVRNPKILLLDEATSALDTSSEAKVQKALDKAQEGRTTIVVAHRLSTIRNVDVIYVFKAGLVVECGNHTELMASKGHYYDMVMLQNLPGVDEQSPEKTKLSRETSIISEKDDEDEFLEFRNDVKEDAAEAPDISFMRVLKLNKPEWKSVTLASICSLMSGFCMPLFAVIFGDFLALLDGDDPDEIQKGVSRLALIFVGIGVFSGITNFIVVFFYGIAGEALTKRLRLMMFRKLLEMDIGFYDDKDNSTGALCARLSGEAAAVQGATGQRIGTVVQAVGTFGFALVLSLIFEWRVGLVALTFVPIIIFVLYKEGRMTYAATSGTVKVMETSSKIAVEAVANVRTVASLGREETFRREYSKQLRPALDTAVRSSHWRGLVFGMSRGVFNFVIASSLYYGGTIIVNEGVPFEQVFKSAQALLMGATSAAQAFAFAPNFQKGIKAAGRVIVLLGRQSKITDPAEPAVKNFNGTGEASLQGIQFRYPTRPLVRVLKDLNLEIQRGKTVALVGASGCGKSTVIQLLERYYDPEDGIVAQDGVPLPKLNLVDARRAIGFVQQEPILFDRTIGENIAYGNNEARVSNDEVIEAAQQANIHNFITSLPLGYDTNIGSKGTQLSGGQKQRVAIARALIRRPKMLLLDEATSALDTESEKVVQEALDKAKAGRTCVMIAHRLSTVRDADVICVIHEGQVAEMGTHNELLELKGLYYNLNRRGYA

SEQ ID NO: 44 - protein, 25 aa, Spodoptera frugiperda
YLLEAMVNYSIFGDADAFMKSLLNF

SEQ ID NO: 45 - DNA, 4178 bp, Bacillus thuringiensis
ttacaattcaagatgaattgcaggtaaatggttctaacatgtataagtgtaagtatttctacattaccacaaattctcaatttgtatatgtaaaataggaaaagtggattttatatataagtataaaaagtaataagactttaaaataagttaacggaatacaaacccttaatgcattggttaaacattgtaaagtctaaagcatggataatgggcgagaagtaagtagattgttaacaccctgggtcaaaaattgatatttagtaaaattagttgcactttgtgcattttttcataagatgagtcatatgttttaaattgtagtaatgaaaaacagtattatatcataatgaattggtatcttaataaaagagatggaggtaacttatggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtataatcaatttagaagagaattaacactaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctcctatgttctcttggatacatcgtagtgctgaatttaataatataattgcatcggatagtattactcaaatccctgcagtgaagggaaactttctttttaatggttctgtaatttcaggaccaggatttactggtggggacttagttagattaaatagtagtggaaataacattcagaatagagggtatattgaagttccaattcacttcccatcgacatctaccagatatcgagttcgtgtacggtatgcttctgtaaccccgattcacctcaacgttaattggggtaattcatccattttttccaatacagtaccagctacagctacgtcattagataatctacaatcaagtgattttggttattttgaaagtgccaatgcttttacatcttcattaggtaatatagtaggtgttagaaattttagtgggactgcaggagtgataatagacagatttgaatttattccagttactgcaacactcgaggctgaatataatctggaaagagcgcagaaggcggtgaatgcgctgtttacgtctacaaaccaactagggctaaaaacaaatgtaacggattatcatattgatcaagtgtccaatttagttacgtatttatcggatgaattttgtctggatgaaaagcgagaattgtccgagaaagtcaaacatgcgaagcgactcagtgatgaacgcaatttactccaagattcaaatttcaaagacattaataggcaaccagaacgtgggtggggcggaagtacagggattaccatccaaggaggggatgacgtatttaaagaaaattacgtcacactatcaggtacctttgatgagtgctatccaacatatttgtatcaaaaaatcgatgaatcaaaattaaaagcctttacccgttatcaattaagagggtatatcgaagatagtcaagacttagaaatctatttaattcgctacaatgcaaaacatgaaacagtaaatgtgccaggtacgggttccttatggccgctttcagcccaaagtccaatcggaaagtgtggagagccgaatcgatgcgcgccacaccttgaatggaatcctgacttagattgttcgtgtagggatggagaaaagtgtgcccatcattcgcatcatttctccttagacattgatgtaggatgtacagacttaaatgaggacctaggtgtatgggtgatctttaagattaagacgcaagatgggcacgcaagactagggaatctagagtttctcgaagagaaaccattagtaggagaagcgctagctcgtgtgaaaagagcggagaaaaaatggagagacaaacgtgaaaaattggaatgggaaacaaatatcgtttataaagaggcaaaagaatctgtagatgctttatttgtaaactctcaatatgatcaattacaagcggatacgaatattgccatgattcatgcggcagataaacgtgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaacgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgcgtagaagaggaaatctatccaaataacacggtaacgtgtaatgattatactgtaaatcaagaagaatacggaggtgcgtacacttctcgtaatcgaggatataacgaagctccttccgtaccagctgattatgcgtcagtctatgaagaaaaatcgtatacagatggacgaagagagaatccttgtgaatttaacagagggtatagggattacacgccactaccagttggttatgtgacaaaagaattagaatacttcccagaaaccgataaggtatggattgagattggagaaacggaaggaacatttatcgtggacagcgtggaattactccttatggaggaatagtctcatgcaaactcaggtttaaatatcgttttcaaatcaattgtccaagagcagcattacaaatagataagtaatttgttgtaatgaaaaacggacatcacctccattgaaacggagtgatgtccgttttactatgttattttctagtaatacatatgtatagagcaacttaatcaagcagagatattttcacctatcgatgaaaatatctctgctttttctttttttatttggtatatgctttacttgtaatc

SEQ ID NO: 46 - DNA, 2983 bp, Bacillus thuringiensis
aagcttaattaaagataatatctttgaattgtaacgcccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatttatgaaaaatcattttatcagtttgaaaattatgtattatgataagaaagggaggaagaaaaatgaatccgaacaatcgaagtgaacatgatacaataaaaactactgaaaataatgaggtgccaactaaccatgttcaatatcctttagcggaaactccaaatccaacactagaagatttaaattataaagagtttttaagaatgactgcagataataatacggaagcactagatagctctacaacaaaagatgtcattcaaaaaggcatttccgtagtaggtgatctcctaggcgtagtaggtttcccgtttggtggagcgcttgtttcgttttatacaaactttttaaatactatttggccaagtgaagacccgtggaaggcttttatggaacaagtagaagcattgatggatcagaaaatagctgattatgcaaaaaataaagctcttgcagagttacagggccttcaaaataatgtcgaagattatgtgagtgcattgagttcatggcaaaaaaatcctgtgagttcacgaaatccacatagccaggggcggataagagagctgttttctcaagcagaaagtcattttcgtaattcaatgccttcgtttgcaatttctggatacgaggttctatttctaacaacatatgcacaagctgccaacacacatttatttttactaaaagacgctcaaatttatggagaagaatggggatacgaaaaagaagatattgctgaattttataaaagacaactaaaacttacgcaagaatatactgaccattgtgtcaaatggtataatgttggattagataaattaagaggttcatcttatgaatcttgggtaaactttaaccgttatcgcagagagatgacattaacagtattagatttaattgcactatttccattgtatgatgttcggctatacccaaaagaagttaaaaccgaattaacaagagacgttttaacagatccaattgtcggagtcaacaaccttaggggctatggaacaaccttctctaatatagaaaattatattcgaaaaccacatctatttgactatctgcatagaattcaatttcacacgcggttccaaccaggatattatggaaatgactctttcaattattggtccggtaattatgtttcaactagaccaagcataggatcaaatgatataatcacatctccattctatggaaataaatccagtgaacctgtacaaaatttagaatttaatggagaaaaagtctatagagccgtagcaaatacaaatcttgcggtctggccgtccgctgtatattcaggtgttacaaaagtggaatttagccaatataatgatcaaacagatgaagcaagtacacaaacgtacgactcaaaaagaaatgttggcgcggtcagctgggattctatcgatcaattgcctccagaaacaacagatgaacctctagaaaagggatatagccatcaactcaattatgtaatgtgctttttaatgcagggtagtagaggaacaatcccagtgttaacttggacacataaaagtgtagacttttttaacatgattgattcgaaaaaaattacacaacttccgttagtaaaggcatataagttacaatctggtgcttccgttgtcgcaggtcctaggtttacaggaggagatatcattcaatgcacagaaaatggaagtgcggcaactatttacgttacaccggatgtgtcgtactctcaaaaatatcgagctagaattcattatgcttctacatctcagataacatttacactcagtttagacggggcaccatttaatcaatactatttcgataaaacgataaataaaggagacacattaacgtataattcatttaatttagcaagtttcagcacaccattcgaattatcagggaataacttacaaataggcgtcacaggattaagtgctggagataaagtttatatagacaaaattgaatttattccagtgaattaaattaactagaaagtaaagaagtagtgaccatctatgatagtaagcaaaggataaaaaaatgagttcataaaatgaataacatagtgttcttcaactttcgctttttgaaggtagatgaagaacactatttttattttcaaaatgaaggaagttttaaatatgtaatcatttaaagggaacaatgaaagtaggaaataagtcattatctataacaaaataacatttttatatagccagaaatgaattataatattaatcttttctaaattgacgtttttctaaacgttctatagcttcaagacgcttagaatcatcaatatttgtatacagagctgttgtttccatcgagttatgtcccatttgattcgctaatagaacaagatctttattttcgttataatgattggttgcataagtatggcgtaatttatgagggcttttcttttcatcaaaagccctcgtgtatttctctgtaagctt

SEQ ID NO: 47 - DNA, 2370 bp, Bacillus thuringiensis
atgaacaagaataatactaaattaagcacaagagccttaccaagttttattgattattttaatggcatttatggatttgccactggtatcaaagacattatgaacatgatttttaaaacggatacaggtggtgatctaaccctagacgaaattttaaagaatcagcagttactaaatgatatttctggtaaattggatggggtgaatggaagcttaaatgatcttatcgcacagggaaacttaaatacagaattatctaaggaaatattaaaaattgcaaatgaacaaaatcaagttttaaatgatgttaataacaaactcgatgcgataaatacgatgcttcgggtatatctacctaaaattacctctatgttgagtgatgtaatgaaacaaaattatgcgctaagtctgcaaatagaatacttaagtaaacaattgcaagagatttctgataagttggatattattaatgtaaatgtacttattaactctacacttactgaaattacacctgcgtatcaaaggattaaatatgtgaacgaaaaatttgaggaattaacttttgctacagaaactagttcaaaagtaaaaaaggatggctctcctgcagatattcttgatgagttaactgagttaactgaactagcgaaaagtgtaacaaaaaatgatgtggatggttttgaattttaccttaatacattccacgatgtaatggtaggaaataatttattcgggcgttcagctttaaaaactgcatcggaattaattactaaagaaaatgtgaaaacaagtggcagtgaggtcggaaatgtttataacttcttaattgtattaacagctctgcaagcaaaagcttttcttactttaacaacatgccgaaaattattaggcttagcagatattgattatacttctattatgaatgaacatttaaataaggaaaaagaggaatttagagtaaacatcctccctacactttctaatactttttctaatcctaattatgcaaaagttaaaggaagtgatgaagatgcaaagatgattgtggaagctaaaccaggacatgcattgattgggtttgaaattagtaatgattcaattacagtattaaaagtatatgaggctaagctaaaacaaaattatcaagtcgataaggattccttatcggaagttatttatggtgatatggataaattattgtgcccagatcaatctgaacaaatctattatacaaataacatagtatttccaaatgaatatgtaattactaaaattgatttcactaaaaaaatgaaaactttaagatatgaggtaacagcgaatttttatgattcttctacaggagaaattgacttaaataagaaaaaagtagaatcaagtgaagcggagtatagaacgttaagtgctaatgatgatggggtgtatatgccgttaggtgtcatcagtgaaacatttttgactccgattaatgggtttggcctccaagctgatgaaaattcaagattaattactttaacatgtaaatcatatttaagagaactactgctagcaacagacttaagcaataaagaaactaaattgatcgtcccgccaagtggttttattagcaatattgtagagaacgggtccatagaagaggacaatttagagccgtggaaagcaaataataagaatgcgtatgtagatcatacaggcggagtgaatggaactaaagctttatatgttcataaggacggaggaatttcacaatttattggagataagttaaaaccgaaaactgagtatgtaatccaatatactgttaaaggaaaaccttctattcatttaaaagatgaaaatactggatatattcattatgaagatacaaataataatttagaagattatcaaactattaataaacgttttactacaggaactgatttaaagggagtgtatttaattttaaaaagtcaaaatggagatgaagcttggggagataactttattattttggaaattagtccttctgaaaagttattaagtccagaattaattaatacaaataattggacgagtacgggatcaactaatattagcggtaatacactcactctttatcagggaggacgagggattctaaaacaaaaccttcaattagatagtttttcaacttatagagtgtatttttctgtgtccggagatgctaatgtaaggattagaaattctagggaagtgttatttgaaaaaagatatatgagcggtgctaaagatgtttctgaaatgttcactacaaaatttgagaaagataacttttatatagagctttctcaagggaataatttatatggtggtcctattgtacatttttacgatgtctctattaagtaa

SEQ ID NO: 48 - protein, 299 aa, Artificial Sequence
  SYNTHESIZED
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSGSHHHHHHGSGSLEVLFQGPLPTLPWSVCQEEWGDCLPSDPDLDPVGTITNETKSSAELYFLKTVLQQKDGIEDGLG

SEQ ID NO: 49 - protein, 448 aa, Artificial Sequence
  SYNTHESIZED
MGSGSHHHHHHGSGSNLYFQGGEFLDRLSATDEDGLHAGRVTFSIAGNDEAAEYFNVLNDGDNSAMLTLKQALPAGVQQFELVIRATDGGTEPGPRSTDCSVTVVFVMTQGDPVFDDNAASVRFVEKEAGMSEKFQLPQADDPKNYRCMDDCHTIYYSIVDGNDGDHFAVEPETNVIYLLKPLDRSQQEQYRVVVAASNTPGGTSTLSSSLLTVTIGVREANPRPIFESEFYTAGVLHTDSIHKELVYLAAKHSEGLPIVYSIDQETMKIDESLQTVVEDAFDINSATGVISLNFQPTSVMHGSFDFEVVASDTRGASDRAKVSIYMISTRVRVAFLFYNTEAEVNERRNFIAQTFANAFGMTCNIDSVLPATDANGVIREGYTELQAHFIRDDQPVPADYIEGLFTELNTLRDIREVLSTQQLTLLDFAAGGSAVLPGGEYALAVYI

SEQ ID NO: 50 - protein, 642 aa, Artificial Sequence
  SYNTHESIZED
MNNVLNSGRTTICDAYNVVAHDPFSFEHKSLDTIQKEWMEWKRTDHSLYVAPVVGTVSSFLLKKVGSLIGKRILSELWGIIFPSGSTNLMQDILRETEQFLNQRLNTDTLARVNAELIGLQANIREFNQQVDNFLNPTQNPVPLSITSSVNTMQQLFLNRLPQFQIQGYQLLLLPLFAQAANMHLSFIRDVILNADEWGISAATLRTYRDYLRNYTRDYSNYCINTYQTAFRGLNTRLHDMLEFRTYMFLNVFEYVSIWSLFKYQSLMVSSGRTYPIQTSSQLTREIYTSSVIEDSPVSANIPNGFNRAEFGVRPPHLMDFMNSLFVTAETVRSQTVWGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIEIYAANENGTMIHLAPEDYTGFTISPIHATQVNNQTRTFISEKFGNQGDSLRFEQSNTTARYTLRGNGNSYNLYLRVSSIGNSTIRVTINGRVYTVSNVNTTTNNDGVNDNGARFSDINIGNIVASDNTNVTLDINVTLNSGTPFDLMNIMFVPTNLPPLY

SEQ ID NO: 51 - protein, 1176 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLLAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFSNYDSRRYPIRTVSQLTREIYTNPVLENFDGSFRGMAQRIEQNIRQPHLMDILNSITIYTDVHRGFNYWSGHQITASPVGFSGPEFAFPLFGNAGNAAPPVLVSLTGLGIFRTLSSPLYRRIILGSGPNNQELFVLDGTEFSFASLTTNLPSTIYRQRGTVDSLDVIPPQDNSVPPRAGFSHRLSHVTMLSQAAGAVYTLRAPTFSWQHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKQELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

SEQ ID NO: 52 - protein, 652 aa, Bacillus thuringiensis
MNPNNRSEHDTIKVTPNSELQTNHNQYPLADNPNSTLEELNYKEFLRMTEDSSTEVLDNSTVKDAVGTGISVVGQILGVVGVPFAGALTSFYQSFLNTIWPSDADPWKAFMAQVEVLIDKKIEEYAKSKALAELQGLQNNFEDYVNALNSWKKTPLSLRSKRSQDRIRELFSQAESHFRNSMPSFAVSKFEVLFLPTYAQAANTHLLLLKDAQVFGEEWGYSSEDVAEFYHRQLKLTQQYTDHCVNWYNVGLNGLRGSTYDAWVKFNRFRREMTLTVLDLIVLFPFYDIRLYSKGVKTELTRDIFTDPIFSLNTLQEYGPTFLSIENSIRKPHLFDYLQGIEFHTRLQPGYFGKDSFNYWSGNYVETRPSIGSSKTITSPFYGDKSTEPVQKLSFDGQKVYRTIANTDVAAWPNGKVYLGVTKVDFSQYDDQKNETSTQTYDSKRNNGHVSAQDSIDQLPPETTDEPLEKAYSHQLNYAECFLMQDRRGTIPFFTWTHRSVDFFNTIDAEKITQLPVVKAYALSSGASIIEGPGFTGGNLLFLKESSNSIAKFKVTLNSAALLQRYRVRIRYASTTNLRLFVQNSNNDFLVIYINKTMNKDDDLTYQTFDLATTNSNMGFSGDKNELIIGAESFVSNEKIYIDKIEFIPVQL

SEQ ID NO: 53 - protein, 789 aa, Bacillus thuringiensis
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 54 - protein, 18 aa, Artificial Sequence
  SYNTHESIZED
GSTSGSGKPGSGEGSTKG

SEQ ID NO: 55 - DNA, 54 bp, Artificial Sequence
  SYNTHESIZED
ggctcaacttctggatctgggaagccagggagcggcgaaggttcgactaagggc

SEQ ID NO: 56 - protein, 17 aa, Artificial Sequence
  SYNTHESIZED
AEAAAKEAAAKEAAAKA

SEQ ID NO: 57 - DNA, 51 bp, Artificial Sequence
  SYNTHESIZED
gcggaggccgctgcaaaggaagctgctgcgaaggaagcagcggccaaggcg

SEQ ID NO: 58 - protein, 17 aa, Artificial Sequence
  SYNTHESIZED
ESGSVSSEQLAQFRSLD

SEQ ID NO: 59 - DNA, 51 bp, Artificial Sequence
  SYNTHESIZED
gaaagcggcagcgtgagcagcgaacagctggcgcagtttcgcagcctggat

SEQ ID NO: 60 - protein, 15 aa, Artificial Sequence
  SYNTHESIZED
GGGGSGGGGSGGGGS

SEQ ID NO: 61 - DNA, 45 bp, Artificial Sequence
  SYNTHESIZED
ggtggcggcgggagtggcgggggtggtagtggcggtgggggctcc

SEQ ID NO: 62 - protein, 8 aa, Artificial Sequence
  SYNTHESIZED
GGGGGGGG

SEQ ID NO: 63 - DNA, 24 bp, Artificial Sequence
  SYNTHESIZED
ggtggcggcgggggcgggggtggt

SEQ ID NO: 64 - protein, 12 aa, Artificial Sequence
  SYNTHESIZED
PTPTTPTPTTPT

SEQ ID NO: 65 - DNA, 36 bp, Artificial Sequence
  SYNTHESIZED
ccgactccgactactcccacacctaccacgcccacc

SEQ ID NO: 66 - protein, 20 aa, Artificial Sequence
  SYNTHESIZED
MKYLLPTAAAGLLLLAAQPA

SEQ ID NO: 67 - DNA, 402 bp, Artificial Sequence
  SYNTHESIZED
atggcccaggtgcagctgcaggagtctgggggaggcttggtgcagccgggggggtctctgagactctcctgtgcagcctctggattcaccttcagtagcgctgccatgagctgggtccgccaggctccaggaaaggggctcgagtgggtctcagttattaatagtggcggtgatagtacaagttatacaggctccgtgaagggccgattcaccatctccagggacaacgccaaggcgacactgtatctgcaaatgaacaacctgaaacctgaggacacggccgtgtattattgtgcaagagggagtacccgcggccaggggacccaggtcaccgtctcctcagcggccgcatacccgtacgacgttccggactacggttcccaccaccatcaccatcactag

SEQ ID NO: 68 - protein, 133 aa, Artificial Sequence
  SYNTHESIZED
MAQVQLQESGGGLVQPGGSLRLSCAASGFTFSSAAMSWVRQAPGKGLEWVSVINSGGDSTSYTGSVKGRFTISRDNAKATLYLQMNNLKPEDTAVYYCARGSTRGQGTQVTVSSAAAYPYDVPDYGSHHHHHH

SEQ ID NO: 69 - DNA, 444 bp, Artificial Sequence
  SYTHESIZED
atggcccaggtgcagctgcaggagtctgggggaggctcggtgcagcctggggggtctctaaaactctcctgtgcagcctctggattcaccttcagtaactatgccatgagctgggtccgccaggctccaggaaaggggctcgagtgggtctcaggtattaatgctggtggtgatacgacaaactatgcagactccgtgaaggaccgattcaccatctccagagacaacgccaagaacacgctgtatctccaaatgaacagcctgaaacctgaggacacggccatatattactgtctaaagcttgagaccagtgtggttcctagtagtagttactaccgcaatcgcggttccaggggccagggaacccaggtcaccgtctcctcagcggccgcatacccgtacgacgttccggactacggttcccaccaccatcaccatcactag

SEQ ID NO: 70 - protein, 147 aa, Artificial Sequence
  SYNTHESIZED
MAQVQLQESGGGSVQPGGSLKLSCAASGFTFSNYAMSWVRQAPGKGLEWVSGINAGGDTTNYADSVKDRFTISRDNAKNTLYLQMNSLKPEDTAIYYCLKLETSVVPSSSYYRNRGSRGQGTQVTVSSAAAYPYDVPDYGSHHHHHH

SEQ ID NO: 71 - DNA, 444 bp, Artificial Sequence
  SYNTHESIZED
atggcccaggtgcagctgcaggagtctgggggaggattggtgcaggctggggactctctgagactctcctgtgcagcctctgaacaatccttcaatagcgaaattatgggctggttccgccaggctccagggaaggagcgtgagtttgtagcagctattacctatagtggtagtatcacaaaatatgcagactccgcgaagggccgattcaccatctccagagacaacgccaagaacacggtgtatctgcaaatgaacagtttgaaccctgaggacacggccctttattactgtgcccttaacaaagggggactgtatactgactaccgatcttgggcgacgtatgactaccgcggccaggggacccaggtcaccgtctcctcagcggccgcatacccgtacgacgttccggactacggttcccaccaccatcaccatcactag

SEQ ID NO: 72 - protein, 147 aa, Artificial Sequence
  SYNTHESIZED
MAQVQLQESGGGLVQAGDSLRLSCAASEQSFNSEIMGWFRQAPGKEREFVAAITYSGSITKYADSAKGRFTISRDNAKNTVYLQMNSLNPEDTALYYCALNKGGLYTDYRSWATYDYRGQGTQVTVSSAAAYPYDVPDYGSHHHHHH

SEQ ID NO: 73 - DNA, 429 bp, Artificial Sequence
  SYNTHESIZED
atggcccaggtgcagctgcaggagtctgggggaggattggtgcaggctgggggctctctgagactctcctgtgcagcctctggacgcaccttcagtaggtatgccatgggctggtttcgccaggctctagggaaggagcgtgagttcgtagcaggtattaactggagtggtagtatgacatactatgcagactccgtgaagggccgattcaccatctccagagacaacgccaagaacatgctgtatctgcaaatgaacagtctgaaatctgaggacacggccgtgtattactgtgccggcgtgacggtagtaggtggtgcaccagcctttgactactggggccaggggacccaggtcaccgtctcctcagcggccgcatacccgtacgacgttccggactacggttcccaccaccatcaccatcactag

SEQ ID NO: 74 - protein, 142 aa, Artificial Sequence
  SYNTHESIZED
MAQVQLQESGGGLVQAGGSLRLSCAASGRTFSRYAMGWFRQALGKEREFVAGINWSGSMTYYADSVKGRFTISRDNAKNMLYLQMNSLKSEDTAVYYCAGVTVVGGAPAFDYWGQGTQVTVSSAAAYPYDVPDYGSHHHHHH

SEQ ID NO: 75 - DNA, 417 bp, Artificial Sequence
  SYNTHESIZED
atggcccaggtgcagctgcaggagtctggaggaggagtggtgcagactgggggctccctgacactctcctgtaaagcctctagacgcaccagtggctttgccatggcctggttccgccaggctccagggatggaacgtgaatttgtagcgggcattggtcggactggggataatatccactatttagattctgtgaagggccgattcaccatctctagagataataccaagaacacgctgtctctgcaaatgaacagcctgagacctggggacacggccgtctattactgtgcagcagacgtgacaaagagtggatttatttattggggccaggggacccaggtcaccgtctcctcagcggccgcatacccgtacgacgttccggactacggttcccaccaccatcaccatcactag

SEQ ID NO: 76 - protein, 138 aa, Artificial Sequence
  SYNTHESIZED
MAQVQLQESGGGVVQTGGSLTLSCKASRRTSGFAMAWFRQAPGMEREFVAGIGRTGDNIHYLDSVKGRFTISRDNTKNTLSLQMNSLRPGDTAVYYCAADVTKSGFIYWGQGTQVTVSSAAAYPYDVPDYGSHHHHHH

SEQ ID NO: 77 - DNA, 444 bp, Artificial Sequence
  SYNTHESIZED
atggcccaggtgcagctgcaggagtctgggggaggcttggtgcagcctggggggtctctgagactcgcctgtgtactctccggaggcccctcgagtagttatggtgtgggctggttccgacagcgatcagggacagagcgtgaatttgtagcagctatcagtgggagtggtcgtactatccattatgtagacgacgtgaagggccgattcgccatctccagagacagcgccaagaatgcggtggatctgcaaatgaacaacctgaaacctgaggacacggccgtttattactgtgcggcactcgcgctcgttactactcatccgacgagcaatgtgggtgaatgggactactggggccaggggacccaggtcaccgtctcctcagcggccgcatacccgtacgacgttccggactacggttcccaccaccatcaccatcactag

SEQ ID NO: 78 - protein, 147 aa, Artificial Sequence
  SYNTHESIZED
MAQVQLQESGGGLVQPGGSLRLACVLSGGPSSSYGVGWFRQRSGTEREFVAAISGSGRTIHYVDDVKGRFAISRDSAKNAVDLQMNNLKPEDTAVYYCAALALVTTHPTSNVGEWDYWGQGTQVTVSSAAAYPYDVPDYGSHHHHHH

SEQ ID NO: 79 - DNA, 426 bp, Artificial Sequence
  SYNTHESIZED
atggcccaggtgcagctgcaggagtctggaggaggagtggtgcagactgggggctccctgacactctcctgtaaagcctctagacgcaccagtggctttgccatggcctggttccgccaggctccagggatggaacgtgaatttgtagcgggcattggtcggactggggataatatccactatttagattctgtgaagggccgattcaccatctctagagataataccaagaacacgctgtctctgcaaatgaacagtctgaaatctgaggacacggccgtgtattactgtgcaaaagtggtggtagtagctggttcaccatctttcgacgcatggggccaggggacccaggtcaccgtctcctcagcggccgcatacccgtacgacgttccggactacggttcccaccaccatcaccatcactag

SEQ ID NO: 80 - protein, 141 aa, Artificial Sequence
  SYNTHESIZED
MAQVQLQESGGGVVQTGGSLTLSCKASRRTSGFAMAWFRQAPGMEREFVAGIGRTGDNIHYLDSVKGRFTISRDNTKNTLSLQMNSLKSEDTAVYYCAKVVVVAGSPSFDAWGQGTQVTVSSAAAYPYDVPDYGSHHHHHH

SEQ ID NO: 81 - DNA, 423 bp, Artificial Sequence
  SYNTHESIZED
atggcccaggtgcagctgcaggagtctgggggaggattggtgcaggctgggggctctctgacgctctcctgcgcattctctggtcgcaccttcactcattatgccatggcctggttccgccaggctccagggaaggagcgtaagttcgtagctggtgttacacggagtggcccgaacacatattatgacgactccgtgcagggccgattcaccatctcaagagacaacgccaagaacacggtttatctgcacatgaacagcctgaaacctgaggacacggccgtttattactgtgctgcaaattcgggggtagtatccggatatgactactggggccaggggacccaggtcaccgtctcctcagcggccgcatacccgtacgacgttccggactacggttcccaccaccatcaccatcactag

SEQ ID NO: 82 - protein, 140 aa, Artificial Sequence
  SYNTHESIZED
MAQVQLQESGGGLVQAGGSLTLSCAFSGRTFTHYAMAWFRQAPGKERKFVAGVTRSGPNTYYDDSVQGRFTISRDNAKNTVYLHMNSLKPEDTAVYYCAANSGVVSGYDYWGQGTQVTVSSAAAYPYDVPDYGSHHHHHH

SEQ ID NO: 83 - DNA, 414 bp, Artificial Sequence
  SYNTHESIZED
atggcccaggttcagttgcaggaaagcggtggcggcgtagttcaaactggcggttcgctgacactttcttgtaaggcttctcgtcgcacttccgggtttgcaatggcctggttccgtcaggctccggggatggagcgtgagtttgtcgcggggattgggcgcacgggggacaacatccattaccttgattcggtcaaaggccgcttcactatttctcgtgataacacaaagaacactctgagcttacaaatgaataatcttaaaccggaggacacggctgtttattattgcttgcgtactatgggagggacctggtcggagaaggggcaaggcacgcaggtcacggttagctcagcggccgcatacccgtacgacgttccggactacggttcccaccaccatcaccatcactag

SEQ ID NO: 84 - protein, 137 aa, Artificial Sequence
  SYNTHESIZED
MAQVQLQESGGGVVQTGGSLTLSCKASRRTSGFAMAWFRQAPGMEREFVAGIGRTGDNIHYLDSVKGRFTISRDNTKNTLSLQMNNLKPEDTAVYYCLRTMGGTWSEKGQGTQVTVSSAAAYPYDVPDYGSHHHHHH

SEQ ID NO: 85 - DNA, 453 bp, Artificial Sequence
  SYNTHESIZED
atggcccaggtgcagctgcaggagtctgggggaggattggtgcaggctggggcctctctgagactctcctgtgcagcctctagacgcaccggcagtagtcttaccatgggctggttccgccaggctccagggaaggagcgtgagtttgtagcagctattagccggagtggtattagaacatactacgcagactttgtgaagggccggttcaccatctccagagacaacgccaagaacacgctctatctgcaaatgaacagcctgaaacctgaggacacggccgtgtattactgtgcggctaacgacaaaacatacggtagtggtcttgacgtctacacaaggcgacaaaactattactactggggcccggggacccaggtcaccgtctcctcagcggccgcatacccgtacgacgttccggactacggttcccaccaccatcaccatcactag

SEQ ID NO: 86 - protein, 150 aa, Artificial Sequence
  SYNTHESIZED
MAQVQLQESGGGLVQAGASLRLSCAASRRTGSSLTMGWFRQAPGKEREFVAAISRSGIRTYYADFVKGRFTISRDNAKNTLYLQMNSLKPEDTAVYYCAANDKTYGSGLDVYTRRQNYYYWGPGTQVTVSSAAAYPYDVPDYGSHHHHHH

SEQ ID NO: 87 - DNA, 453 bp, Artificial Sequence
  SYNTHESIZED
atggcccaggtgcagctgcaggagtctggaggaggattggcgcaggctggggcctctctgagactctcctgtgcagcctctagacgcaccggcagtagtcttaccatgggctggttccgccaggctccagggaaggagcgtgagtttgtagcagctattagccggagtggtattagaacatactacgcagactttgtgaagggccggttcaccatctccagagacaacgccaagaacacgctctatctgcaaatgaacagcctgaaacctgaggacacggccgtgtattactgtgcggctaacgacaaaacatacggtagtggtcttgacgtctacacaaggcgacaagactattactactggggcccggggacccaggtcaccgtctcctcagcggccgcatacccgtacgacgttccggactacggttcccaccaccatcaccatcactag

SEQ ID NO: 88 - protein, 150 aa, Artificial Sequence
  SYNTHESIZED
MAQVQLQESGGGLAQAGASLRLSCAASRRTGSSLTMGWFRQAPGKEREFVAAISRSGIRTYYADFVKGRFTISRDNAKNTLYLQMNSLKPEDTAVYYCAANDKTYGSGLDVYTRRQDYYYWGPGTQVTVSSAAAYPYDVPDYGSHHHHHH

SEQ ID NO: 89 - DNA, 414 bp, Artificial Sequence
  SYNTHESIZED
atggcccaggtgcagctgcaggagtctgggggaggctcggtgcaggctggggggtctctgaaactctcctgtgcagcttctggaaccgaaagcattttcatcgtgatgggctggtaccgccaggctccagggaaagagcgcgagttggtcgcggcaatgtcatttggtggtggtacaaatgttacagacggcgtgaggggccgattcaccatctccagagactttgacaagaacacggtggatctacaaatgaacaacctaaaaactgaggacacggccgtctattactgtaatgcagtccagtggggccctcgtgactactggggccaggggacccaggtcaccgtctcctcagcggccgcatacccgtacgacgttccggactacggttcccaccaccatcaccatcactag

SEQ ID NO: 90 - protein, 137 aa, Artificial Sequence
  SYNTHESIZED
MAQVQLQESGGGSVQAGGSLKLSCAASGTESIFIVMGWYRQAPGKERELVAAMSFGGGTNVTDGVRGRFTISRDFDKNTVDLQMNNLKTEDTAVYYCNAVQWGPRDYWGQGTQVTVSSAAAYPYDVPDYGSHHHHHH

SEQ ID NO: 91 - DNA, 453 bp, Artificial Sequence
  SYNTHESIZED
atggcccaggtgcagctgcaggagtctggaggaggattggtgcaggctggggcctctctgagactctcctgtgcagcctctagacgcaccggcagtagtcttaccatgggctggttccgccaggctccagggagggagcgtgagtttgtagcagctattagccggagtggtattagaacatactacgcagactttgtgaagggccggttcaccatctccagagacaacgccaagaacacgctctatctgcaaatgaacagcctgaaacctgaggacacggccgtgtattactgtgcggctaacgacaaaacatacggtagtggtcttgacgtctacacaaggcgacaagactattactactggggcccggggacccaggtcaccgtctcctcagcggccgcatacccgtacgacgttccggactacggttcccaccaccatcaccatcactag

SEQ ID NO: 92 - protein, 150 aa, Artificial Sequence
  SYNTHESIZED
MAQVQLQESGGGLVQAGASLRLSCAASRRTGSSLTMGWFRQAPGREREFVAAISRSGIRTYYADFVKGRFTISRDNAKNTLYLQMNSLKPEDTAVYYCAANDKTYGSGLDVYTRRQDYYYWGPGTQVTVSSAAAYPYDVPDYGSHHHHHH

SEQ ID NO: 93 - DNA, 441 bp, Artificial Sequence
  SYNTHESIZED
atggcccaggtgcagctgcaggagtctgggggaggattggtgcaggctggggcctctctgagactctcctgtgcagcctctagacgcaccggcagtagtcttaccatgggctggttccgccaggctccagggaaggagcgtgagtttgtagcagctattagccggagtggtattagaacatactacgcagactttgtgaagggccggttcaccatctccagagataacggcgagaatacgctgtatctgcaaatcaacagcctgaaacctgaggacacggccgtttattactgtgcggcatccagcaacccgggatactatcgtacagctcccaaccagtataacttctggggcccggggacccaggtcaccgtctcctctgcggccgcatacccgtacgacgttccggactacggttcccaccaccatcaccatcactag

SEQ ID NO: 94 - protein, 146 aa, Artificial Sequence
  SYNTHESIZED
MAQVQLQESGGGLVQAGASLRLSCAASRRTGSSLTMGWFRQAPGKEREFVAAISRSGIRTYYADFVKGRFTISRDNGENTLYLQINSLKPEDTAVYYCAASSNPGYYRTAPNQYNFWGPGTQVTVSSAAAYPYDVPDYGSHHHHHH

SEQ ID NO: 95 - DNA, 828 bp, Artificial Sequence
  SYNTHESIZED
atggcccaggtgcagctgcaggagtctgggggaggattggtgcaggctggggactctctgagactctcctgtgcagcctctgaacaatccttcaatagcgaaattatgggctggttccgccaggctccagggaaggagcgtgagtttgtagcagctattacctatagtggtagtatcacaaaatatgcagactccgcgaagggccgattcaccatctccagagacaacgccaagaacacggtgtatctgcaaatgaacagtttgaaccctgaggacacggccctttattactgtgcccttaacaaagggggactgtatactgactaccgatcttgggcgacgtatgactaccgcggccaggggacccaggtcaccgtctcctcaccgactccgactactcccacacctaccacgcccacccaggtgcagctgcaggagtctggaggaggagtggtgcagactgggggctccctgacactctcctgtaaagcctctagacgcaccagtggctttgccatggcctggttccgccaggctccagggatggaacgtgaatttgtagcgggcattggtcggactggggataatatccactatttagattctgtgaagggccgattcaccatctctagagataataccaagaacacgctgtctctgcaaatgaacaacctgaaacctgaggacacggccgtgtattactgcctacgtacgatgggtggtacctggtctgagaagggccaggggacccaggtcaccgtctcctcagcggccgcatacccgtacgacgttccggactacggttcccaccaccatcaccatcactag

SEQ ID NO: 96 - protein, 275 aa, Artificial Sequence
  SYNTHESIZED
MAQVQLQESGGGLVQAGDSLRLSCAASEQSFNSEIMGWFRQAPGKEREFVAAITYSGSITKYADSAKGRFTISRDNAKNTVYLQMNSLNPEDTALYYCALNKGGLYTDYRSWATYDYRGQGTQVTVSSPTPTTPTPTTPTQVQLQESGGGVVQTGGSLTLSCKASRRTSGFAMAWFRQAPGMEREFVAGIGRTGDNIHYLDSVKGRFTISRDNTKNTLSLQMNNLKPEDTAVYYCLRTMGGTWSEKGQGTQVTVSSAAAYPYDVPDYGSHHHHHH

SEQ ID NO: 97 - DNA, 867 bp, Artificial Sequence
  SYNTHESIZED
atggcccaggtgcagctgcaggagtctgggggaggattggtgcaggctggggactctctgagactctcctgtgcagcctctgaacaatccttcaatagcgaaattatgggctggttccgccaggctccagggaaggagcgtgagtttgtagcagctattacctatagtggtagtatcacaaaatatgcagactccgcgaagggccgattcaccatctccagagacaacgccaagaacacggtgtatctgcaaatgaacagtttgaaccctgaggacacggccctttattactgtgcccttaacaaagggggactgtatactgactaccgatcttgggcgacgtatgactaccgcggccaggggacccaggtcaccgtctcctcaccgacaccgaccacccccacgcccactactcccacccaagtccagttacaggagtcaggaggtggtctggtccaagcaggtgccagtttgcgtcttagttgcgcggcctcacgccgtactggtagtagtctgactatggggtggtttcgccaagccccaggtcgtgaacgtgaatttgtcgcagcgattagccgtagcggtatccgtacctattatgcggactttgtcaagggccgtttcactatctcccgcgacaatgcgaaaaacactttgtaccttcaaatgaactcattgaaaccagaggataccgcggtgtactattgtgcggcgaacgacaagacgtatgggtcggggctggatgtatatacgcgtcgtcaggattattactactggggtcccggtacccaggtaacagtgtcatcagcggccgcatacccgtacgacgttccggactacggttcccaccaccatcaccatcactag

SEQ ID NO: 98 - protein, 288 aa, Artificial Sequence
  SYNTHESIZED
MAQVQLQESGGGLVQAGDSLRLSCAASEQSFNSEIMGWFRQAPGKEREFVAAITYSGSITKYADSAKGRFTISRDNAKNTVYLQMNSLNPEDTALYYCALNKGGLYTDYRSWATYDYRGQGTQVTVSSPTPTTPTPTTPTQVQLQESGGGLVQAGASLRLSCAASRRTGSSLTMGWFRQAPGREREFVAAISRSGIRTYYADFVKGRFTISRDNAKNTLYLQMNSLKPEDTAVYYCAANDKTYGSGLDVYTRRQDYYYWGPGTQVTVSSAAAYPYDVPDYGSHHHHHH

SEQ ID NO: 99 - DNA, 867 bp, Artificial Sequence
  SYNTHESIZED
atggcccaggtgcagctgcaggagtctgggggaggattggtgcaggctggggactctctgagactctcctgtgcagcctctgaacaatccttcaatagcgaaattatgggctggttccgccaggctccagggaaggagcgtgagtttgtagcagctattacctatagtggtagtatcacaaaatatgcagactccgcgaagggccgattcaccatctccagagacaacgccaagaacacggtgtatctgcaaatgaacagtttgaaccctgaggacacggccctttattactgtgcccttaacaaagggggactgtatactgactaccgatcttgggcgacgtatgactaccgcggccaggggacccaggtcaccgtctcctcaccgacgccgacaacccctacaccgactactccgactcaagttcaattacaggaatcaggcggaggcttggtacaggctggtgcatcgttacgtctttcttgcgcagcttcccgtcgcacgggctcttctttaacgatgggctggtttcgtcaggcgccgggcaaagagcgtgaattcgtggcggccatctcgcgtagcgggattcgtacttattatgccgactttgtcaaaggccgcttcactattagtcgtgataatgcaaagaacaccctttacctgcaaatgaatagcttgaagccggaagacactgccgtttactactgcgcggcaaacgacaagacatatgggtctggccttgacgtttatacccgtcgtcaaaattattattattgggggcctggtactcaagttaccgtgtcgtcagcggccgcatacccgtacgacgttccggactacggttcccaccaccatcaccatcactag

SEQ ID NO: 100 - protein, 288 aa, Artificial Sequence
  SYNTHESIZED
MAQVQLQESGGGLVQAGDSLRLSCAASEQSFNSEIMGWFRQAPGKEREFVAAITYSGSITKYADSAKGRFTISRDNAKNTVYLQMNSLNPEDTALYYCALNKGGLYTDYRSWATYDYRGQGTQVTVSSPTPTTPTPTTPTQVQLQESGGGLVQAGASLRLSCAASRRTGSSLTMGWFRQAPGKEREFVAAISRSGIRTYYADFVKGRFTISRDNAKNTLYLQMNSLKPEDTAVYYCAANDKTYGSGLDVYTRRQNYYYWGPGTQVTVSSAAAYPYDVPDYGSHHHHHH

SEQ ID NO: 101 - DNA, 855 bp, Artificial Sequence
  SYNTHESIZED
atggcccaggtgcagctgcaggagtctgggggaggattggtgcaggctggggactctctgagactctcctgtgcagcctctgaacaatccttcaatagcgaaattatgggctggttccgccaggctccagggaaggagcgtgagtttgtagcagctattacctatagtggtagtatcacaaaatatgcagactccgcgaagggccgattcaccatctccagagacaacgccaagaacacggtgtatctgcaaatgaacagtttgaaccctgaggacacggccctttattactgtgcccttaacaaagggggactgtatactgactaccgatcttgggcgacgtatgactaccgcggccaggggacccaggtcaccgtctcctcaccgacccccacaacgccaacgccgactactccgacacaagtacaattacaagaatcaggaggaggtttagttcaagctggcgcttctttacgtttaagttgtgcggcctcccgtcgtaccggctcctcattgacaatggggtggtttcgtcaggctcctgggaaggagcgtgaatttgtcgccgcaattagccgttctggcattcgtacctactacgcagattttgtcaaggggcgtttcaccatttcacgcgataacggggagaatacgctttacttgcagatcaattctctgaagcctgaagataccgcggtgtactattgcgccgcctcttctaatccgggttattatcgcacagcaccaaaccaatataacttctggggtcctgggacacaagtgacggtttcttctgcggccgcatacccgtacgacgttccggactacggttcccaccaccatcaccatcactag

SEQ ID NO: 102 - protein, 284 aa, Artificial Sequence
  SYNTHESIZED
MAQVQLQESGGGLVQAGDSLRLSCAASEQSFNSEIMGWFRQAPGKEREFVAAITYSGSITKYADSAKGRFTISRDNAKNTVYLQMNSLNPEDTALYYCALNKGGLYTDYRSWATYDYRGQGTQVTVSSPTPTTPTPTTPTQVQLQESGGGLVQAGASLRLSCAASRRTGSSLTMGWFRQAPGKEREFVAAISRSGIRTYYADFVKGRFTISRDNGENTLYLQINSLKPEDTAVYYCAASSNPGYYRTAPNQYNFWGPGTQVTVSSAAAYPYDVPDYGSHHHHHH

SEQ ID NO: 103 - DNA, 855 bp, Artificial Sequence
  SYNTHESIZED
atggcccaggtgcagctgcaggagtctgggggaggattggtgcaggctggggactctctgagactctcctgtgcagcctctgaacaatccttcaatagcgaaattatgggctggttccgccaggctccagggaaggagcgtgagtttgtagcagctattacctatagtggtagtatcacaaaatatgcagactccgcgaagggccgattcaccatctccagagacaacgccaagaacacggtgtatctgcaaatgaacagtttgaaccctgaggacacggccctttattactgtgcccttaacaaagggggactgtatactgactaccgatcttgggcgacgtatgactaccgcggccaggggacccaggtcaccgtctcctcaggtggcggcggaggtggaggtggtcaggtgcagcttcaagaatcaggggggggattggcacaagccggggctagcttacgcctgtcctgcgcggcgagccgccgtacaggctcaagtcttacgatgggctggtttcgtcaggcacctgggaaagagcgtgagtttgttgccgccatctcgcgctcgggtatccgtacctactatgcagatttcgttaaaggacgcttcacgatttctcgcgataatgctaaaaacacgctgtatctgcagatgaattccttaaagccggaagatacagcggtgtactactgtgcggcaaatgataagacctatggttctggtctggatgtttatacacgtcgtcaagattattactactgggggcccggcacgcaggttactgtgtcgtcagcggccgcatacccgtacgacgttccggactacggttcccaccaccatcaccatcactag

SEQ ID NO: 104 - protein, 284 aa, Artificial Sequence
  SYNTHESIZED
MAQVQLQESGGGLVQAGDSLRLSCAASEQSFNSEIMGWFRQAPGKEREFVAAITYSGSITKYADSAKGRFTISRDNAKNTVYLQMNSLNPEDTALYYCALNKGGLYTDYRSWATYDYRGQGTQVTVSSGGGGGGGGQVQLQESGGGLAQAGASLRLSCAASRRTGSSLTMGWFRQAPGKEREFVAAISRSGIRTYYADFVKGRFTISRDNAKNTLYLQMNSLKPEDTAVYYCAANDKTYGSGLDVYTRRQDYYYWGPGTQVTVSSAAAYPYDVPDYGSHHHHHH

SEQ ID NO: 105 - DNA, 816 bp, Artificial Sequence
  SYNTHESIZED
atggcccaggtgcagctgcaggagtctgggggaggattggtgcaggctggggactctctgagactctcctgtgcagcctctgaacaatccttcaatagcgaaattatgggctggttccgccaggctccagggaaggagcgtgagtttgtagcagctattacctatagtggtagtatcacaaaatatgcagactccgcgaagggccgattcaccatctccagagacaacgccaagaacacggtgtatctgcaaatgaacagtttgaaccctgaggacacggccctttattactgtgcccttaacaaagggggactgtatactgactaccgatcttgggcgacgtatgactaccgcggccaggggacccaggtcaccgtctcctcaggtggtggcggcggaggaggcggacaggtgcagcttcaagagtcaggaggcggttctgtacaagccgggggatctctgaaattaagttgcgcagcctctgggacagagtcgatttttattgttatgggctggtatcgtcaggcacctggcaaggaacgtgagctggtcgccgccatgagttttgggggcgggacgaatgtgactgacggagttcgtggtcgctttaccatttcccgtgattttgacaagaacacggtcgacctgcaaatgaacaatcttaaaaccgaggacaccgcagtttactattgtaatgctgtgcaatggggcccgcgcgactactggggacagggcactcaggtcacagtatcctcagcggccgcatacccgtacgacgttccggactacggttcccaccaccatcaccatcactag

SEQ ID NO: 106 - protein, 271 aa, Artificial Sequence
  SYNTHESIZED
MAQVQLQESGGGLVQAGDSLRLSCAASEQSFNSEIMGWFRQAPGKEREFVAAITYSGSITKYADSAKGRFTISRDNAKNTVYLQMNSLNPEDTALYYCALNKGGLYTDYRSWATYDYRGQGTQVTVSSGGGGGGGGQVQLQESGGGSVQAGGSLKLSCAASGTESIFIVMGWYRQAPGKERELVAAMSFGGGTNVTDGVRGRFTISRDFDKNTVDLQMNNLKTEDTAVYYCNAVQWGPRDYWGQGTQVTVSSAAAYPYDVPDYGSHHHHHH

SEQ ID NO: 107 - DNA, 843 bp, Artificial Sequence
  SYNTHESIZED
atggcccaggtgcagctgcaggagtctgggggaggctcggtgcagcctggggggtctctaaaactctcctgtgcagcctctggattcaccttcagtaactatgccatgagctgggtccgccaggctccaggaaaggggctcgagtgggtctcaggtattaatgctggtggtgatacgacaaactatgcagactccgtgaaggaccgattcaccatctccagagacaacgccaagaacacgctgtatctccaaatgaacagcctgaaacctgaggacacggccatatattactgtctaaagcttgagaccagtgtggttcctagtagtagttactaccgcaatcgcggttccaggggccagggaacccaggtcaccgtctcctcaccgacgcctactaccccgactccgaccactcctactcaggtgcagcttcaggagtcaggtggaggtcttgttcaggctggtggatctcttcgcttgtcttgcgctgcgtcgggtcgtacattctcgcgctacgcgatgggctggttccgtcaggctttgggtaaagagcgcgagtttgtggcagggattaactggtccgggtctatgacgtactacgcagattctgttaaaggtcgcttcactatttctcgtgataacgcgaaaaacatgttgtatctgcaaatgaattctcttaaatcggaggataccgcagtttactattgcgcaggagtcaccgtcgtcggaggtgcgccagcttttgactattggggtcaaggcactcaagtaaccgtctcatcagcggccgcatacccgtacgacgttccggactacggttcccaccaccatcaccatcactag

SEQ ID NO: 108 - protein, 280 aa, Artificial Sequence
  SYNTHESIZED
MAQVQLQESGGGSVQPGGSLKLSCAASGFTFSNYAMSWVRQAPGKGLEWVSGINAGGDTTNYADSVKDRFTISRDNAKNTLYLQMNSLKPEDTAIYYCLKLETSVVPSSSYYRNRGSRGQGTQVTVSSPTPTTPTPTTPTQVQLQESGGGLVQAGGSLRLSCAASGRTFSRYAMGWFRQALGKEREFVAGINWSGSMTYYADSVKGRFTISRDNAKNMLYLQMNSLKSEDTAVYYCAGVTVVGGAPAFDYWGQGTQVTVSSAAAYPYDVPDYGSHHHHHH

SEQ ID NO: 109 - DNA, 831 bp, Artificial Sequence
  SYNTHESIZED
atggcccaggtgcagctgcaggagtctgggggaggctcggtgcagcctggggggtctctaaaactctcctgtgcagcctctggattcaccttcagtaactatgccatgagctgggtccgccaggctccaggaaaggggctcgagtgggtctcaggtattaatgctggtggtgatacgacaaactatgcagactccgtgaaggaccgattcaccatctccagagacaacgccaagaacacgctgtatctccaaatgaacagcctgaaacctgaggacacggccatatattactgtctaaagcttgagaccagtgtggttcctagtagtagttactaccgcaatcgcggttccaggggccagggaacccaggtcaccgtctcctcaccgaccccaaccactccgacgcccacaacgcctactcaggtacaattacaggaatcagggggtggtgtagttcaaacggggggctcattgactctttcttgcaaagcgtctcgccgtacctccggttttgcgatggcctggttccgccaagcaccagggatggaacgtgaatttgttgccggtattggtcgcactggtgataacatccactaccttgactcggtgaagggtcgtttcaccatttcccgcgacaacaccaaaaataccttatccttgcaaatgaattctcttcgtccaggtgataccgccgtttattactgtgctgctgatgtcactaagtcgggcttcatctactggggtcagggcacacaggtcaccgtttcatcagcggccgcatacccgtacgacgttccggactacggttcccaccaccatcaccatcactag

SEQ ID NO: 110 - protein, 276 aa, Artificial Sequence
  SYNTHESIZED
MAQVQLQESGGGSVQPGGSLKLSCAASGFTFSNYAMSWVRQAPGKGLEWVSGINAGGDTTNYADSVKDRFTISRDNAKNTLYLQMNSLKPEDTAIYYCLKLETSVVPSSSYYRNRGSRGQGTQVTVSSPTPTTPTPTTPTQVQLQESGGGVVQTGGSLTLSCKASRRTSGFAMAWFRQAPGMEREFVAGIGRTGDNIHYLDSVKGRFTISRDNTKNTLSLQMNSLRPGDTAVYYCAADVTKSGFIYWGQGTQVTVSSAAAYPYDVPDYGSHHHHHH

SEQ ID NO: 111 - DNA, 837 bp, Artificial Sequence
  SYNTHESIZED
atggcccaggtgcagctgcaggagtctgggggaggctcggtgcagcctggggggtctctaaaactctcctgtgcagcctctggattcaccttcagtaactatgccatgagctgggtccgccaggctccaggaaaggggctcgagtgggtctcaggtattaatgctggtggtgatacgacaaactatgcagactccgtgaaggaccgattcaccatctccagagacaacgccaagaacacgctgtatctccaaatgaacagcctgaaacctgaggacacggccatatattactgtctaaagcttgagaccagtgtggttcctagtagtagttactaccgcaatcgcggttccaggggccagggaacccaggtcaccgtctcctcaccgacacccacgactcccactcccaccactcctacccaagtgcagttacaggaatcaggtggtggactggtccaagcaggcgggtcccttacgctttcatgtgcgttcagcggacgtacgtttacacactatgctatggcgtggttccgccaagcacctgggaaagaacgtaaattcgtagcaggggtaacccgctcaggaccgaatacttattatgatgactccgtccaagggcgtttcactatcagccgtgataatgcaaagaatactgtgtaccttcacatgaattccttgaagcctgaagacacggcggtatattattgcgctgccaattccggtgtggtttctggatatgattactgggggcaaggcacgcaagtcacggtgtcgtcagcggccgcatacccgtacgacgttccggactacggttcccaccaccatcaccatcactag

SEQ ID NO: 112 - protein, 278 aa, Artificial Sequence
  SYNTHESIZED
MAQVQLQESGGGSVQPGGSLKLSCAASGFTFSNYAMSWVRQAPGKGLEWVSGINAGGDTTNYADSVKDRFTISRDNAKNTLYLQMNSLKPEDTAIYYCLKLETSVVPSSSYYRNRGSRGQGTQVTVSSPTPTTPTPTTPTQVQLQESGGGLVQAGGSLTLSCAFSGRTFTHYAMAWFRQAPGKERKFVAGVTRSGPNTYYDDSVQGRFTISRDNAKNTVYLHMNSLKPEDTAVYYCAANSGVVSGYDYWGQGTQVTVSSAAAYPYDVPDYGSHHHHHH

SEQ ID NO: 113 - DNA, 846 bp, Artificial Sequence
  SYNTHESIZED
atggcccaggtgcagctgcaggagtctgggggaggattggtgcaggctggggactctctgagactctcctgtgcagcctctgaacaatccttcaatagcgaaattatgggctggttccgccaggctccagggaaggagcgtgagtttgtagcagctattacctatagtggtagtatcacaaaatatgcagactccgcgaagggccgattcaccatctccagagacaacgccaagaacacggtgtatctgcaaatgaacagtttgaaccctgaggacacggccctttattactgtgcccttaacaaagggggactgtatactgactaccgatcttgggcgacgtatgactaccgcggccaggggacccaggtcaccgtctcctcaggtgggggaggaggtggtggtggtcaggtacagttgcaggagagtggaggcggtcttgtgcaacctggcgggtcgttgcgcttagcttgtgtattgtcgggcggtccctcgtcgtcctatggggtagggtggtttcgtcagcgcagtggcacagaacgcgaattcgtggctgctatctctgggtccggtcgcacaatccattatgttgacgatgtaaaaggacgcttcgcaatttcacgcgatagcgctaagaatgcagtggacttgcagatgaacaatctgaaaccggaggatacagcagtttattactgtgctgctcttgcactggtaacgacgcatccgacgtctaacgtcggagaatgggactactggggtcaaggcacccaggttaccgtgagttcagcggccgcatacccgtacgacgttccggactacggttcccaccaccatcaccatcactag

SEQ ID NO: 114 - protein, 281 aa, Artificial Sequence
  SYNTHESIZED
MAQVQLQESGGGLVQAGDSLRLSCAASEQSFNSEIMGWFRQAPGKEREFVAAITYSGSITKYADSAKGRFTISRDNAKNTVYLQMNSLNPEDTALYYCALNKGGLYTDYRSWATYDYRGQGTQVTVSSGGGGGGGGQVQLQESGGGLVQPGGSLRLACVLSGGPSSSYGVGWFRQRSGTEREFVAAISGSGRTIHYVDDVKGRFAISRDSAKNAVDLQMNNLKPEDTAVYYCAALALVTTHPTSNVGEWDYWGQGTQVTVSSAAAYPYDVPDYGSHHHHHH

SEQ ID NO: 115 - DNA, 828 bp, Artificial Sequence
  SYNTHESIZED
atggcccaggtgcagctgcaggagtctgggggaggattggtgcaggctggggactctctgagactctcctgtgcagcctctgaacaatccttcaatagcgaaattatgggctggttccgccaggctccagggaaggagcgtgagtttgtagcagctattacctatagtggtagtatcacaaaatatgcagactccgcgaagggccgattcaccatctccagagacaacgccaagaacacggtgtatctgcaaatgaacagtttgaaccctgaggacacggccctttattactgtgcccttaacaaagggggactgtatactgactaccgatcttgggcgacgtatgactaccgcggccaggggacccaggtcaccgtctcctcaggtggcggaggtggaggcggaggtcaagttcaacttcaggaatccggtgggggtgtagtacaaacgggagggtctcttacgctgtcctgcaaggcatctcgccgcacctcagggttcgctatggcctggtttcgtcaagcgccagggatggaacgcgagttcgttgctggaatcggacgtactggtgacaacatccattatcttgacagtgtgaaaggacgcttcaccatcagccgcgataatacaaaaaacaccttaagccttcaaatgaattctctgaagagcgaggacaccgcagtttattattgtgccaaagtggttgttgtagcggggtcacctagtttcgacgcgtggggccagggcacgcaggttaccgtgtcctcagcggccgcatacccgtacgacgttccggactacggttcccaccaccatcaccatcactag

SEQ ID NO: 116 - protein, 275 aa, Artificial Sequence
  SYNTHESIZED
MAQVQLQESGGGLVQAGDSLRLSCAASEQSFNSEIMGWFRQAPGKEREFVAAITYSGSITKYADSAKGRFTISRDNAKNTVYLQMNSLNPEDTALYYCALNKGGLYTDYRSWATYDYRGQGTQVTVSSGGGGGGGGQVQLQESGGGVVQTGGSLTLSCKASRRTSGFAMAWFRQAPGMEREFVAGIGRTGDNIHYLDSVKGRFTISRDNTKNTLSLQMNSLKSEDTAVYYCAKVVVVAGSPSFDAWGQGTQVTVSSAAAYPYDVPDYGSHHHHHH

SEQ ID NO: 117 - DNA, 453 bp, Artificial Sequence
  SYNTHESIZED
atggcccaggtgcagctgcaggagtctgggggaggattggtgcaggctggggcctctctgagactctcctgtgcagcctctagacgcaccggcagtagtcttaccatgggctggttccgccaggctccagggaaggagcgtgagtttgtagcagctattagccggagtggtattagaacatactacgcagactttgtgaagggccggttcaccatctccagagacaacgccaagaacacgctctatctgcaaatgaacagcctgaaacctggggacacggccgtgtattactgtgcggctaacgacaaaacatacggtagtggtcttgacgtctacacaaggcgacaagactattactactggggcccggggacccaggtcaccgtctcctcagcggccgcatacccgtacgacgttccggactacggttcccaccaccatcaccatcactag

SEQ ID NO: 118 - protein, 150 aa, Artificial Sequence
  SYNTHESIZED
MAQVQLQESGGGLVQAGASLRLSCAASRRTGSSLTMGWFRQAPGKEREFVAAISRSGIRTYYADFVKGRFTISRDNAKNTLYLQMNSLKPGDTAVYYCAANDKTYGSGLDVYTRRQDYYYWGPGTQVTVSSAAAYPYDVPDYGSHHHHHH

SEQ ID NO: 119 - DNA, 414 bp, Artificial Sequence
  SYNTHESIZED
atggcccaggtgcagctgcaggagtctgggggaggctcggtgcaggctggggggtctctgaaactctcctgtgcagcctctggaaccgaaagcattttcatcgtgatgggctggtaccgccaggctccagggaaagagcgcgagttggtcgcggcaatgacatttggtggtggtacaaatgtgacagacggcgtgaggggccgattcaccatctccagagaccttgacaagaacacggtggatctgcaaatgaacaacctaaaaactgaggacacggccgtctattactgtaatgcagtccgttggggccctcgtgactactggggccaggggacccaggtcaccgtctcctcagcggccgcatacccgtacgacgttccggactacggttcccaccaccatcaccatcactag

SEQ ID NO: 120 - protein, 137 aa, Artificial Sequence
  SYNTHESIZED
MAQVQLQESGGGSVQAGGSLKLSCAASGTESIFIVMGWYRQAPGKERELVAAMTFGGGTNVTDGVRGRFTISRDLDKNTVDLQMNNLKTEDTAVYYCNAVRWGPRDYWGQGTQVTVSSAAAYPYDVPDYGSHHHHHH

SEQ ID NO: 121 - DNA, 450 bp, Artificial Sequence
  SYNTHESIZED
atggcccaggtgcagctgcaggagtctgggggaggattggtgcaggctgggggctctctgagactctcctgtgcagcctctggacgcactttcagtagcgcgcccatggcctggttccgccaggttccagggaaggagcgtgaatttgtcgccgctattagcagtaatgatggtagtacaaggtatggagactccgtgaagggccgattcaccatctccagagacaacgccaagaacgcgctgtggctgcaaatgaacagcctgaaacctgaggacacggccgtgtattactgtgcggcccggcgaacattacggagtggtagttacacgcccggcgcaacatattactatgactcatggggcccggggacccaggtcaccgtctcctcagcggccgcatacccgtacgacgttccggactacggttcccaccaccatcaccatcactag

SEQ ID NO: 122 - protein, 149 aa, Artificial Sequence
  SYNTHESIZED
MAQVQLQESGGGLVQAGGSLRLSCAASGRTFSSAPMAWFRQVPGKEREFVAAISSNDGSTRYGDSVKGRFTISRDNAKNALWLQMNSLKPEDTAVYYCAARRTLRSGSYTPGATYYYDSWGPGTQVTVSSAAAYPYDVPDYGSHHHHHH

SEQ ID NO: 123 - DNA, 432 bp, Artificial Sequence
  SYNTHESIZED
atggcccaggtgcagctgcaggagtctggaggaggcttggtgcaggctggggggtctctgagactctcctgtgtagcctctggaagcatcttcagtggcgatgccatgggctggtaccgccaggctccagggaagcagcgcgagttggtcgcaactattactcgtggtgggattacaaactatgccgactccgcgaagggccgattcaccatctccagagacaatgccaagaacacggtgtctctgcaaatgaacagcctgaaacctgaggacacggccgtctattactgtcatgcagaagacccgggttggggtgtctaccgggggcgtcgtggctactggggccaggggacccaggtcaccgtctcctcagcggccgcatacccgtacgacgttccggactacggttcccaccaccatcaccatcactag

SEQ ID NO: 124 - protein, 141 aa, Artificial Sequence
  SYNTHESIZED
QVQLQESGGGLVQAGGSLRLSCVASGSIFSGDAMGWYRQAPGKQRELVATITRGGITNYADSAKGRFTISRDNAKNTVSLQMNSLKPEDTAVYYCHAEDPGWGVYRGRRGYWGQGTQVTVSSAAAYPYDVPDYGSHHHHHH

SEQ ID NO: 125 - DNA, 453 bp, Artificial Sequence
  SYNTHESIZED
atggcccaggtgcagctgcaggagtctggaggaggattggtgcaggctggggcctctctgagactctcctgtgcagcctctagacgcaccggcagtagtcttaccatgggctggttccgccaggctccagggaaggagcgtgagtttgtagcagctattagccggagtggtattagaacatactacgcaggctttgtgaagggccggttcaccatctccagagacaacgccaagaacacgctctatctgcaaatgaacagcctgaaacctgaggacacggccgtgtattactgtgcggctaacgacaaaacatacggtagtggtcttgacgtctacacaaggcgacaagactattactactggggcccggggacccaggtcaccgtctcctcagcggccgcatacccgtacgacgttccggactacggttcccaccaccatcaccatcactag

SEQ ID NO: 126 - protein, 150 aa, Artificial Sequence
  SYNTHESIZED
MAQVQLQESGGGLVQAGASLRLSCAASRRTGSSLTMGWFRQAPGKEREFVAAISRSGIRTYYAGFVKGRFTISRDNAKNTLYLQMNSLKPEDTAVYYCAANDKTYGSGLDVYTRRQDYYYWGPGTQVTVSSAAAYPYDVPDYGSHHHHHH

SEQ ID NO: 127 - DNA, 414 bp, Artificial Sequence
  SYNTHESIZED
atggcccaggtgcagctgcaggagtctgggggaggctcggtgcaggctggggggtctctgaaactctcctgtgcagcctctggaaccgaaagcattttcatcgtgatgggctggtaccgccaggctccagggaaagagcgcgaattggtcgcggcaatgtcatttggtggtggagcaaatgttacagacggcgtgaggggccgattcaccatctccagagaccttgacaagaacacggtagatctgcaaatgaacaacctaaaacctgaggacacggccgtctattactgtaatgcagtccggtggggccctcgtgactactggggtcaggggacccaggtcaccgtctcctcagcggccgcatacccgtacgacgttccggactacggttcccaccaccatcaccatcactag

SEQ ID NO: 128 - protein, 137 aa, Artificial Sequence
  SYNTHESIZED
MAQVQLQESGGGSVQAGGSLKLSCAASGTESIFIVMGWYRQAPGKERELVAAMSFGGGANVTDGVRGRFTISRDLDKNTVDLQMNNLKPEDTAVYYCNAVRWGPRDYWGQGTQVTVSSAAAYPYDVPDYGSHHHHHH

SEQ ID NO: 129 - DNA, 411 bp, Artificial Sequence
  SYNTHESIZED
atggcccaagttcaactgcaggagtccggcgggggtgtagtgcagactggaggcagcttgacgctttcctgtaaggcttcacgtcgcacgtctggatttgcgatggcctggttccgtcaggcccctgggatggaacgcgagttcgttgctggtatcgggcgcacgggtgataacatccactacttggattctgttaaagggcgtttcgctatttcccgtgacaacacaaagaatacgttatatcttcagatgaataatttgcagccagaggatacagcggtctattactgctgcttggctaaagctgctgatcctttttgcgatcaaggtactcaggtaacggtctcctcagcggccgcatacccgtacgacgttccggactacggttcccaccaccatcaccatcactag

SEQ ID NO: 130 - protein, 136 aa, Artificial Sequence
  SYNTHESIZED
MAQVQLQESGGGVVQTGGSLTLSCKASRRTSGFAMAWFRQAPGMEREFVAGIGRTGDNIHYLDSVKGRFAISRDNTKNTLYLQMNNLQPEDTAVYYCCLAKAADPFCDQGTQVTVSSAAAYPYDVPDYGSHHHHHH

SEQ ID NO: 131 - DNA, 420 bp, Artificial Sequence
  SYNTHESIZED
atggcccaattgcaagaatcaggtggcggtgttgttcaaaccggaggatcattaacattatcgtgcaaagcgtcccgtcgcacatcaggcttcgcaatggcgtggtttcgtcaggcacctgggatggagcgcgagtttgtggctgggattgggcgcactggtgataatatccattaccttgattcagtaaaaggacgtttcacgatctcacgtgataatactaagaacacgcttagtttacagatgaacaacctgaaaccggaagacactgcagtgtactactgcgcagcgaatccttggatcaatacgggaacgggatggaactactggggacaagggacccaagtgacggtatcttcagcggccgcatacccgtacgacgttccggactacggttcccaccaccatcaccatcactag

SEQ ID NO: 132 - protein, 139 aa, Artificial Sequence
  SYNTHESIZED
MAQLQESGGGVVQTGGSLTLSCKASRRTSGFAMAWFRQAPGMEREFVAGIGRTGDNIHYLDSVKGRFTISRDNTKNTLSLQMNNLKPEDTAVYYCAANPWINTGTGWNYWGQGTQVTVSSAAAYPYDVPDYGSHHHHHH

SEQ ID NO: 133 - DNA, 417 bp, Artificial Sequence
  SYNTHESIZED
atggcccaggtgcagctgcaggagtctgggggaggcttggtgcagcctggggggtctctgagactctcctgtgcagcctctggaagcatcttcagtatccctgccatgggctggtaccgtcaggctccagggaagcagcgcgagttggtcgcagttattactagagatggtagcacgcactatgcagactccgtgaagggccgattcaccatctccagagacaacgccaaaaacacgctgtatctgcaaatgaacagtctgaaatctgaggacacggccgtgtattactgtgccaaactgggtactagccgatcgtatgactactggggccaggggacccaggtcaccgtctcctcagcggccgcatacccgtacgacgttccggactacggttcccaccaccatcaccatcactag

SEQ ID NO: 134 - protein, 138 aa, Artificial Sequence
  SYNTHESIZED
MAQVQLQESGGGLVQPGGSLRLSCAASGSIFSIPAMGWYRQAPGKQRELVAVITRDGSTHYADSVKGRFTISRDNAKNTLYLQMNSLKSEDTAVYYCAKLGTSRSYDYWGQGTQVTVSSAAAYPYDVPDYGSHHHHHH